(19) United States
(12) Patent Application Publication
(10) Pub. No.: US 2014/0004108 A1
(43) Pub. Date: Jan. 2, 2014

(54) Methods for Modulation of Autophagy Through the Modulation of Autophagy-Enhancing Gene Products

(71) Applicant: HARVARD MEDICAL SCHOOL, CAMBRIDGE, MA (US)

(72) Inventors: JUNYING YUAN, NEWTON, MA (US); MARTA M. LIPINSKI, BALTIMORE, MD (US)

(73) Assignee: Harvard Medical School, Cambridge, MA (US)

(21) Appl. No.: 13/929,036

(22) Filed: Jun. 27, 2013

Related U.S. Application Data
(63) Continuation of application No. 13/499,314, filed on Aug. 9, 2012, now abandoned.
(60) Provisional application No. 61/247,251, filed on Sep. 30, 2009.
(60) Provisional application No. 61/247,309, filed on Sep. 30, 2009.

(51) Int. Cl. C12N 5/079

(52) U.S. Cl. CPC C12N 5/0618
USPC 424/133.1; 435/375

(57) ABSTRACT

The present disclosure relates to methods for the modulation of autophagy and the treatment of autophagy-related diseases, including cancer, neurodegenerative diseases and pancreatitis.

### RELATED APPLICATIONS

This application is a continuation of U.S. patent application Ser. No. 13/499,314, filed Sep. 30, 2010, which is a §371 national stage application based on PCT Application No. PCT/US2010/050968, filed Sep. 30, 2010, which claims the benefit of priority to U.S. Provisional Patent Application No. 61/247,251, filed Sep. 30, 2009 and U.S. Provisional Patent Application No. 61/247,309, filed Sep. 30, 2009 each of which are hereby incorporated by reference in their entirety.

### GOVERNMENT SUPPORT

This invention was made with U.S. Government support under National Institutes of Health Grant Nos. AG012859 and AG027916. The government has certain rights in the invention.

### BACKGROUND

Autophagy is a catabolic process that mediates the turnover of intracellular constituents in a lysosome-dependent manner (Levine and Klionsky, (2004) Dev Cell 6, 463-377). Autophagy is initiated by the formation of an isolation membrane, which expands to engulf a portion of the cytoplasm to form a double membrane vesicle called the autophagosome. The autophagosome then fuses with a lysosome to form an autolysosome, where the captured material and the inner membrane are degraded by lysosomal hydrolases. Autophagy is therefore critical for the clearance of large protein complexes and defective organelles, and plays an important role in cellular growth, survival and homeostasis.

Autophagy has been primarily studied in unicellular eukaryotes, where it is known to be critical for survival of starvation conditions. When a unicellular eukaryote is cultured under conditions of nutrient deprivation, products of autophagic degradation, such as amino acids, fatty acids and nucleotides, can be used by the cell as structural components and as sources of energy (Levine and Klionsky, (2004) Dev Cell 6, 463-377; Levine and Kroemer, (2008), Cell 132, 27-42).

Cells in complex, multicellular eukaryotes, such as mammals, rarely experience nutrient deprivation under normal physiological conditions. However, when such cells undergo nutrient deprivation or cellular stress, autophagy is often upregulated, which enhances cell survival. Because of their rapid growth and genetic instability, cancer cells are more reliant on autophagy for survival and growth than untransformed cells (Ding et al., (2009), Mol. Cancer. Ther., 8(7), 2036-2045). Additionally, autophagy is frequently activated as a survival mechanism in cancer cells in response to the cellular stress caused by chemotherapeutic agents. Autophagy inhibitors therefore can act as anti-cancer therapeutic agents either alone or in combination with other cancer treatments (Maiuri et al., (2007) Nat. Rev. Cell Biol. 8, 741-752; Amaravadi et al., (2007) J. Clin. Invest. 117, 326-336).

In addition to its role in responding to cellular stress, autophagy is an important intracellular mechanism for the maintenance of cellular homeostasis through the turnover of malfunctioning, aged or damaged proteins and organelles (Levine and Kroemer, (2008), Cell 132, 27-42). As a result, reduced levels of autophagy contribute to neurodegeneration by increasing the accumulation of misfolded proteins (Hara et al., (2006), Nature, 441, 885-889; Komatsu et al., (2006), Nature, 441, 880-884). Upregulation of autophagy has been demonstrated to reduce both the levels of aggregated proteins and the symptoms of neurodegenerative diseases (Rubinsztein et al., (2007), Nat. Rev. Drug Discov. 6, 304-312). Agents that enhance cellular autophagy therefore can act as therapeutic agents for the prevention or treatment of neurodegenerative diseases.

In addition to cancer and neurodegeneration, modulation of autophagy is a therapeutic strategy in a wide variety of additional diseases and disorders. For example, several liver diseases, cardiac diseases and muscle diseases are correlated with the accumulation of misfolded protein aggregates. In such diseases, agents that increase cellular autophagy may enhance the clearance of disease-causing aggregates and thereby contribute to treatment and reduce disease severity (Levine and Kroemer, (2008), Cell, 132, 27-42). Additionally, elevated levels of autophagy have also been observed in pancreatic diseases, and have been demonstrated to be an early event in the progression of acute pancreatitis (Fortunato and Kroemer, (2009), Autophagy, 5(6)). Inhibitors of autophagy may, therefore, function as therapeutic agents in the treatment of pancreatitis.

There is therefore abundant evidence indicating that modulation of autophagy is a useful approach for the treatment of a wide range of diseases and disorders. However, because the genes and pathways responsible for the regulation of mammalian autophagy are poorly understood, there are few validated autophagy regulators that can serve as targets for the development of new therapeutic agents and methods for the treatment of such diseases. Accordingly, there is great need for new methods for the modulation of autophagy and treatment of autophagy-associated diseases.

### SUMMARY

The present invention provides novel methods for the modulation of autophagy and the treatment of autophagy-related diseases, including cancer, neurodegenerative diseases, liver diseases, muscle diseases and pancreatitis. In order to identify the methods of the present invention, a high-throughput image-based genome-wide screen of a human siRNA library was used to identify 236 autophagy-related genes. These genes were extensively characterized using a combination of high-throughput assays, low-throughput assays and bioinformatics analysis. Based on the results of these studies, biological and pharmaceutical agents useful in the modulation of these genes and their gene products were identified and novel methods for the modulation of autophagy and the treatment of autophagy-related diseases were developed.

In some embodiments, the invention relates to methods of inducing autophagy in a cell comprising contacting the cell with an agent that inhibits the activity of a product of an autophagy-inhibiting gene of the invention. In certain embodiments, the autophagy-inhibiting gene is selected from the genes listed in Table 1, Table 3, Table 5, Table 7, FIG. 14, FIG. 15, FIG. 39, FIG. 44, and/or FIG. 55. In other embodiments, the autophagy-inhibiting gene is TRPM3, TMPRSS5, IRAK3, ADMR, FGFR1, UNC13B, PTGER2, AGER, BGN, GABBR2, PPARD, GHSR, BAIAIP2, SORCS2, PAQR6, EPHA6, TRHR, C5AR1, BAI3, TLR3, PTPRH, ADRA1A, UTS2R, RORC, CHRND, TACR2, P2RX1, PLXNA2, PTPRU, FCER1A, CD300C, TNFRSF19L CLCF1, LIF, FGF2, SDF1 or IGF. In certain aspects of the invention, the agent is an antibody, a siRNA molecule, a shRNA molecule, and/or an antisense RNA molecule. In other aspects, the agent is TK1258, PF 04494700, PMX53, Tamsulosin, Doxazosin, Prazosin hydrochloride, alfuzosin hydrochloride, Urotensin II, Mecamylamine hydrochloride, ISIS 3521, Gemcitabine, LY900003, MK-5108, U73122 or D609.

Certain embodiments of the invention relate to methods of inhibiting autophagy in a cell comprising contacting the cell with an agent that inhibits the activity of a product of an autophagy-enhancing gene of the invention. In some embodiments, the autophagy-enhancing gene is selected from the genes listed in Table 2, Table 4 and/or Table 6. In other embodiments, the autophagy enhancing gene is TPR, GPR18, RelA or NFκB. In certain embodiments the agent is an antibody, a siRNA molecule, a shRNA molecule, and/or an antisense RNA molecule.

In certain aspects, the invention relates to methods of inhibiting autophagy in a cell comprising contacting the cell with an agent that enhances the activity of a product of an autophagy-inhibiting gene of the invention. In some embodiments, the autophagy-inhibiting gene is selected from the genes listed in Table 1, Table 3, Table 5, Table 7, FIG. 14, FIG. 15, FIG. 39, FIG. 44, and/or FIG. 55. In other embodiments, the autophagy-inhibiting gene is TRPM3, TMPRSS5, IRAK3, ADMR, FGFR1, UNC13B, PTGER2, AGER, BGN, GABBR2, PPARD, GHSR, BAIAIP2, SORCS2, PAQR6, EPHA6, TRHR, C5AR1, BAI3, TLR3, PTPRH, ADRA1A, UTS2R, RORC, CHRND, TACR2, P2RX1, PLXNA2, PTPRU, FCER1A, CD300C, TNFRSF19L CLCF1, LIF, FGF2, SDF1 or IGF. In certain embodiments the agent is an antibody. In some embodiments the agent is FGF-1, acidic FGF-1, XRP0038, RhaFGF, GW501516, Ibutamoren Mesylate, KP-102LN, EP1572, TRH, S-0373, Poly-ICR, CQ-07001 or cryptotanshinone. In some embodiments the agent is a growth factor. In other embodiments, the growth factor is CLCF1, LIF, FGF2, SDF1 or IGF1.

Some embodiments of the invention relate to methods of inducing autophagy in a cell comprising contacting the cell with an agent that enhances the activity of a product of an autophagy-enhancing gene of the invention. In some embodiments, the autophagy-enhancing gene is selected from the genes listed in Table 2, Table 4 and/or Table 6. In other embodiments, the autophagy enhancing gene is TPR, GPR18, RelA or NFκB. In certain embodiments the agent is an antibody.

In some embodiments, the invention relates to methods of treating a neurodegenerative disease and/or a proteinopathy in a subject comprising administering to the subject an agent that inhibits the activity of a product of an autophagy-inhibiting gene of the invention. In certain embodiments, the autophagy-inhibiting gene is selected from the genes listed in Table 1, Table 3, Table 5, Table 7, FIG. 14, FIG. 15, FIG. 39, FIG. 44, and/or FIG. 55. In other embodiments, the autophagy-inhibiting gene is TRPM3, TMPRSS5, IRAK3, ADMR, FGFR1, UNC13B, PTGER2, AGER, BGN, GABBR2, PPARD, GHSR, BAIAIP2, SORCS2, PAQR6, EPHA6, TRHR, C5AR1, BAI3, TLR3, PTPRH, ADRA1A, UTS2R, RORC, CHRND, TACR2, P2RX1, PLXNA2, PTPRU, FCER1A, CD300C, TNFRSF19L CLCF1, SDF1, LIF, FGF2 or IGF. In some embodiments, the agent is an antibody, a siRNA molecule, a shRNA molecule, and/or an antisense RNA molecule. In other embodiments, the agent is TK1258, PF 04494700, PMX53, Tamsulosin, Doxazosin, Prazosin hydrochloride, alfuzosin hydrochloride, Urotensin II, Mecamylamine hydrochloride, ISIS 3521, Gemcitabine, LY900003, MK-5108, U73122 or D609.

Some embodiments of the invention relate to methods of treating a neurodegenerative disease and/or a proteinopathy in a subject comprising administering to the subject an agent that enhances the activity of a product of an autophagy-enhancing gene of the invention. In some embodiments, the autophagy-enhancing gene is selected from the genes listed in Table 2, Table 4 and/or Table 6. In other embodiments, the autophagy enhancing gene is TPR, GPR18, RelA or NFκB. In certain embodiments the agent is an antibody.

In certain embodiments, the neurodegenerative disease is Adrenal Leukodystrophy, alcoholism, Alexander's disease, Alper's disease, Alzheimer's disease, Amyotrophic lateral sclerosis, ataxia telangiectasia, Batten disease, bovine spongiform encephalopathy, Canavan disease, cerebral palsy, cockayne syndrome, corticobasal degeneration, Creutzfeldt-Jakob disease, familial fatal insomnia, frontotemporal lobar degeneration, Huntington's disease, HIV-associated dementia, Kennedy's disease, Krabbe's disease, Lewy body dementia, neuroborreliosis, Machado-Joseph disease, multiple system atrophy, multiple sclerosis, narcolepsy, Niemann Pick disease, Parkinson's disease, Pelizaeus-Merzbacher disease, Pick's disease, primary lateral sclerosis, prion diseases, progressive supranuclear palsy, Refsum's disease, Sandhoff disease, Schilder's disease, subacute combined degeneration of spinal cord secondary to pernicious anaemia, Spielmeyer-Vogt-Sjogren-Batten disease, spinocerebellar ataxia, spinal muscular atrophy, Steele-Richardson-Olszewski disease, Tabes dorsalis, toxic encephalopathy and combinations of these diseases. In some embodiments, the proteinopathy is a 1-antitrypsin deficiency, sporadic inclusion body myositis, limb girdle muscular dystrophy type 2B and Miyoshi myopathy Alzheimer's disease, Parkinson's disease, Lewy Body Dementia, ALS, Huntington's disease, spinocerebellar ataxias, spinobulbar musclular atrophy and combinations of these diseases.

Certain embodiments of the invention relate to methods of treating cancer or pancreatitis in a subject comprising administering to the subject an agent that inhibits the activity of a product of an autophagy-enhancing gene of the invention. In some embodiments, the autophagy-enhancing gene is selected from the genes listed in Table 2, Table 4 and/or Table 6. In other embodiments, the autophagy enhancing gene is TPR, GPR18, RelA or NFκB. In certain embodiments the agent is an antibody, a siRNA molecule, a shRNA molecule, and/or an antisense RNA molecule.

In certain aspects, the invention relates to methods of treating cancer or pancreatitis in a subject comprising administering to the subject an agent that enhances the activity of a product of an autophagy-inhibiting gene of the invention. In some embodiments, the autophagy-inhibiting gene is selected from the genes listed in Table 1, Table 3, Table 5, Table 7, FIG. 14, FIG. 15, FIG. 39, FIG. 44, and/or FIG. 55. In other embodiments, the autophagy-inhibiting gene is TRPM3, TMPRSS5, IRAK3, ADMR, FGFR1, UNC13B, PTGER2, AGER, BGN, GABBR2, PPARD, GHSR, BAIAIP2, SORCS2, PAQR6, EPHA6, TRHR, C5AR1, BAI3, TLR3, PTPRH, ADRA1A, UTS2R, RORC, CHRND, TACR2, P2RX1, PLXNA2, PTPRU, FCER1A, CD300C, TNFRSF19L CLCF1, SDF1, LIF, FGF2 or IGF. In certain embodiments the agent is an antibody. In some embodiments the agent is FGF-1, acidic FGF-1, XRP0038, RhaFGF, GW501516, Ibutamoren Mesylate, KP-102LN, EP1572, TRH, S-0373, Poly-ICR, CQ-07001 or cryptotanshinone. In some embodiments the agent is a growth factor. In more specific embodiments, the growth factor is CLCF1, LIF, FGF2, SDF1 or IGF1.

In some embodiments, the methods of treating cancer further comprise known cancer treatment therapies such as the administration of a chemotherapeutic agent and/or radiation therapy. In certain embodiments the chemotherapeutic agent is altretamine, asparaginase, BCG, bleomycin sulfate, busulfan, camptothecin, carboplatin, carmusine, chlorambucil, cisplatin, claladribine, 2-chlorodeoxyadenosine, cyclophosphamide, cytarabine, dacarbazine imidazole carboxamide, dactinomycin, daunorubicin-dunomycin, dexamethosone, doxurubicin, etoposide, floxuridine, fluorouracil, fluoxymesterone, flutamide, fludarabine, goserelin, hydroxyurea, idarubicin HCL, ifosfamide, interferon α, interferon α 2a, interferon α 2b, interfereon α n3, irinotecan, leucovorin calcium, leuprolide, levamisole, lomustine, megestrol, melphalan, L-sarcosylin, melphalan hydrochloride, MESNA, mechlorethamine, methotrexate, mitomycin, mitoxantrone, mercaptopurine, paclitaxel, plicamycin, prednisone, procarbazine, streptozocin, tamoxifen, 6-thioguanine, thiotepa, topotecan, vinblastine, vincristine or vinorelbine tartrate.

Other embodiments of the invention relate to methods of determining whether an agent is an autophagy inhibitor comprising the step of contacting a cell with the agent, wherein the cell expresses a heterologous autophagy-enhancing gene of the invention, whereby a reduction in autophagy in the cell indicates that the agent is an autophagy inhibitor. In certain aspects, the agent is a small molecule, an antibody, or an inhibitory RNA molecule.

Certain embodiments of the invention relate to methods of determining whether an agent is an autophagy inhibitor, the method comprising the step of contacting a cell with the agent, wherein the expression of an autophagy-inhibiting gene of the invention is inhibited in the cell, whereby a reduction in autophagy in the cell indicates that the agent is an autophagy inhibitor. In certain aspects, the agent is a small molecule, an antibody, or an inhibitory RNA molecule. In some embodiments the cell contains a mutation to the autophagy-related gene. In other embodiments the autophagy-related gene is inhibited by an inhibitory RNA or small molecule.

### BRIEF DESCRIPTION OF THE DRAWINGS

FIG. 1A shows fluorescent microscope images depicting the localization of GFP expressed in H4 cells that stably express LC3-GFP and that were transfected with non-targeting, control siRNA (ntRNA) or siRNA against mTOR or Atg5. FIG. 1B shows the results of a western blot performed using antibodies specific for either LC3 or tubulin and lysates of H4 cells that were transfected with non-targeting, control siRNA (ntRNA) or siRNA against mTOR or Atg5.

FIG. 2 shows the quantification of the level of autophagosome-associated GFP in H4 cells that stably express LC3-GFP and that were transfected with non-targeting, control siRNA (ntRNA) or siRNA against mTOR or Atg5. The asterisks indicate that the difference between the indicated level and that of the ntRNA transfected cells is statistically significant.

FIG. 3 shows the gene symbols, Unigene ID numbers, Genbank accession numbers and names of the autophagy-modulating genes of the invention (Figure discloses ‘DEAD’ as SEQ ID NO: 1).

FIG. 4 shows a schematic diagram depicting a selection of the screens and characterization assays used to identify and characterize the autophagy-modulating genes of the invention.

FIG. 5 shows the quantification of a series of in-cell-western blot assays that measure mTORC1 activity. The asterisks indicate that the difference between the indicated samples and the ntRNA control samples is statistically significant.

FIG. 6 shows the gene symbols, Unigene ID numbers, Genbank accession numbers and names of the genes for which the inhibition of their product results in reduced expression of mTORC.

FIG. 7 shows the gene symbols, Unigene ID numbers, and names of the genes for which the inhibition of their product results in both reduced expression of mTORC and down-regulation of autophagy in the presence of rapamycin.

FIG. 8A shows fluorescent microscope images depicting the localization of RFP expressed in H4 cells that stably express Lamp1-RFP and that were transfected with non-targeting, control siRNA (ntRNA) or siRNA against mTOR. FIG. 8B shows the quantification of the level of autophagosome-associated RFP in H4 cells that stably express LC3-GFP and that were transfected with non-targeting control siRNA (ntRNA) or siRNA against mTOR or Atg5. The asterisks indicate that the difference between the indicated level and that of the ntRNA transfected cells is statistically significant.

FIG. 9 shows the gene symbols, Unigene ID numbers, Genbank accession numbers and names of the genes for which the inhibition of their product result in a significant change in the levels of autophagosome-associated Lamp1-RFP in Lamp1-RFP expressing cells (Figure discloses ‘DEAD’ as SEQ ID NO: 1).

FIG. 10A shows fluorescent microscope images depicting the localization of dsRed expressed in H4 cells that stably express FYVE-dsRed and that were transfected with siRNA against Vprs34 or mTOR. FIG. 10B shows the quantification of the level of autophagosome-associated dsRed in H4 cells that stably express FYVE-dsRed and that were transfected with siRNA against Vprs34 or mTOR. The asterisks indicate that the difference between the indicated level and that of the ntRNA transfected cells is statistically significant. FIG. 10C shows the quantification of the level of autophagosome-associated dsRed in H4 cells that stably express FYVE-dsRed and that were transfected with siRNA against Raptor or mTOR.

FIG. 11 shows the gene symbols, Unigene ID numbers, Genbank accession numbers and names of the genes for which the inhibition of their product results in a significant change in the levels of PtdIns3P levels (Figure discloses ‘DEAD’ as SEQ ID NO: 1).

FIG. 12 shows a Venn diagram depicting the subdivision of genes for which the inhibition of their products led to the induction of autophagy into functional categories based on their dependence on type III PI3 kinase activity, lysosomal function and mTORC1 activity.

FIG. 13 shows the relative average viability of wild-type H4 cells transfected with autophagy-related gene targeting siRNAs (H4) compared to Bcl-2 expressing H4 cells transfected with autophagy-related gene targeting siRNAs (H4+Bcl-2). The asterisks indicate statistical significance.

FIG. 14 shows the relative viability, gene symbols, Unigene ID numbers, and names of the genes for which the inhibition of their product results in enhancement of autophagy in Bcl-2 expressing cells.

FIG. 15 shows the relative viability, gene symbols, Unigene ID numbers, and names of the genes for which the inhibition of their product results in enhancement of autophagy wild-type, but not in Bcl-2 expressing cells.

FIG. 16 shows the quantification of in-cell western assays demonstrating an increase in the levels of GRP78 and GRP94 in H4 cells treated with tunicamycin. The asterisks indicate statistical significance.

FIG. 17 shows the gene symbols, Unigene ID numbers, and names of the genes for which the inhibition of their product results in enhancement of autophagy and changes in Endoplasmic Reticulum (ER) stress levels (Figure discloses ‘DEAD’ as SEQ ID NO: 1).

FIG. 18 shows a western blot depicting Bcl-2 expression in H4 LC3-GFP and H4 FYVE-dsRed cells following infection with pBabe-Bcl-2 retrovirus and puromycin selection.

FIG. 19A shows the quantification of the level of autophagosome-associated GFP in H4 cells that stably express LC3-GFP and Bcl-2 and that were transfected with non-targeting, control siRNA (ntRNA) or siRNA against mTOR. The asterisks indicate that the difference between the indicated level and that of the ntRNA transfected cells is statistically significant. FIG. 19B shows the quantification of the level of autophagosome-associated dsRed in H4 cells that stably express FYVE-dsRed and Bcl-2 and that were transfected with non-targeting, control siRNA (ntRNA) or siRNA against mTOR. The asterisks indicate that the difference between the indicated level and that of the ntRNA transfected cells is statistically significant. FIG. 19C shows the quantification of the level of autophagosome-associated dsRed in H4 cells that stably express FYVE-dsRed and that were transfected with siRNA against autophagy-related gene products that either do not express Bcl-2 (H4) or express Bcl-2 (H4+Bcl-2). The asterisks indicate that the difference between the indicated levels is statistically significant.

FIG. 20 shows the subdivision of autophagy-related genes for which knock-down was able to induce autophagy under conditions of low PtdIns3P into functional categories based on their ability to up-regulate type III PI3 kinase activity or to alter lysosomal function.

FIG. 21A shows how selected autophagy-related gene products of the invention are associated with specific protein complexes. FIG. 21B shows how selected autophagy-related gene products of the invention are associated with a network of transcription factors and chromatin modifying enzymes.

FIG. 22 shows how selected autophagy-related gene products of the invention interact with core autophagic machinery.

FIG. 23 shows how selected autophagy-related gene products of the invention interact within axon-guidance regulatory pathways.

FIG. 24 shows how selected autophagy-related gene products of the invention interact within actin-cytoskeleton regulatory pathways.

FIG. 25A shows the subdivision of the autophagy-related genes of the invention into molecular function categories. FIG. 25B shows the further subdivision of the autophagy-related genes of the invention that are categorized as receptors in FIG. 25A into receptor categories.

FIG. 26 shows the molecular function categories, gene symbols, Unigene ID numbers and gene names of autophagy-related genes of the invention (Figure discloses ‘DEAD’ as SEQ ID NO: 1).

FIG. 27A shows the subdivision of the autophagy-related genes of the invention into biological process categories. FIG. 27B shows the further subdivision of the autophagy-related genes of the invention that are categorized as mediators of signal transduction in FIG. 27A into signal transduction categories.

FIG. 28 shows the quantification of autophagosome associated GFP in H4 LC3-GFP cells grown in the presence of the indicated growth factors (IGF1, FGF2, LIF, CLCF1 and SDF1). The asterisk indicates that the difference between the indicated level and that of the untreated cells is statistically significant.

FIG. 29 shows fluorescent microscope images depicting the localization of GFP expressed in H4 cells that stably express LC3-GFP and that were either untreated under conditions of nutrient deprivation (untreated), untreated under normal growth conditions (serum), or treated with CLCF1, LIF, FGF2 or IGF1 under conditions of nutrient deprivation (CLCF1, LIF, FGF2 and IGF, respectively).

FIG. 30 shows that cytokines are able to suppress autophagy in the absence and presence of rapamycin. H4 cells were grown in serum-free medium, followed by addition of 100 ng/mL IGF1 (A), 50 ng/mL FGF2 (B), 50 ng/mL LIF (C) or 50 ng/mL CLCF1 (D) and 10 μg/mL E64d (E). Where indicated, cells were pre-treated with 50 nM rapamycin 1 hour prior to the addition of cytokines. Levels of autophagy were assessed by western blot using antibody against LC3; mTORC1 activity was evaluated with antibodies against phospho-S6 (Ser235/236, P-S6) and phospho-S6 kinase (Thr389, P-S6K). Quantification of LC3 II/tubulin ratio is shown.

FIG. 31A shows the quantification of autophagosome associated GFP in H4 LC3-GFP cells grown in the presence of 5, 20, 100 or 200 ng/ml of TNFα or the presence of rapamycin. The asterisks indicate that the difference between the indicated level and that of the untreated cells is statistically significant. FIG. 31B shows western blots depicting the levels of p62 in H4 cells that were either untreated under conditions of nutrient deprivation (−), untreated under normal growth conditions (serum), treated with rapamycin (Rap), or treated with 5 ng/ml of TNFα under conditions of nutrient deprivation.

FIG. 32 shows fluorescent microscope images depicting the localization of GFP expressed in H4 cells that stably express LC3-GFP and that were transfected with non-targeting, control siRNA (ntRNA) or four distinct siRNAs specific for RelA.

FIG. 33 shows the quantification of the level of autophagosome-associated GFP in H4 cells that stably express LC3-GFP and that were transfected with non-targeting, control siRNA (ntRNA) or four distinct siRNAs specific for RelA. The asterisks indicate that the difference between the indicated level and that of the ntRNA transfected cells is statistically significant.

FIG. 34A shows the results of semi-quantitative RT-PCR detecting the level of RelA mRNA H4 cells that were transfected with non-targeting, control siRNA (ntRNA) or one of four distinct siRNAs specific for RelA. FIG. 34B shows the results a western blot detecting the level of p65 in H4 cells that were transfected with non-targeting, control siRNA (ntRNA), one of four distinct siRNAs specific for RelA, or a pool of the four RelA specific siRNAs.

FIG. 35A shows western blots depicting the levels of RelA and LC3 in wild-type H4 cells (wt) and RelA^{-/-} and NFκB^{-/-} double knock-out (DKO) H4 cells. FIG. 35B shows western blots depicting the levels of RelA, p62 and LC3 in H4 cells that have been transfected with siRNAs specific for RelA, non-targeting siRNA (nt), mTor or Atg5.

FIG. 36A shows FACS histograms depicting the levels of reactive oxygen species in wild-type H4 cells and RelA^{-/-} and NFκB^{-/-} double knock-out (DKO) H4 cells under normal growth conditions (mock) and conditions of nutrient deprivation (starvation). FIG. 36B shows the quantification of the data depicted in FIG. 36A. FIG. 36C shows the quantification of the levels of reactive oxygen species in H4 cells transfected with non-targeting, control siRNA (ntRNA) or siRNAs specific for RelA grown under normal (+serum) or starvation (HBSS) conditions.

FIG. 37 shows the quantification of the level of autophagosome-associated GFP in H4 cells that stably express LC3-GFP and that were transfected with non-targeting, control siRNA (ntRNA) or siRNAs specific for RelA grown under conditions of nutrient deprivation and either in the presence of antioxidant (NAC) or absence of antioxidant.

FIG. 38 shows the gene symbols, Unigene ID numbers and prediction basis for the autophagy-related genes of the invention whose products are predicted to be localized to the mitochondria.

FIG. 39 shows the gene symbols, Unigene ID numbers and names of autophagy-related genes of the invention with known connections to oxidative damage or the regulation of reactive oxygen species.

FIG. 40A shows western blots depicting the levels of SOD1, p62 and LC3 in H4 cells that were transfected with non-targeting, control siRNA (nt) or siRNA specific for SOD1. FIG. 40B shows fluorescent microscope images depicting the levels of reactive oxygen species in cells transfected with non-targeting, control siRNA (nt) or siRNA specific for SOD1 or treated with 100 mM TBHP. FIG. 40C shows the quantification of the levels of reactive oxygen species in cells transfected with non-targeting, control siRNA (nt) or siRNA specific for SOD1. The asterisks indicate that the difference between the indicated level and that of the ntRNA transfected cells is statistically significant.

FIG. 41 shows the quantification of the level of autophagosome-associated GFP in H4 cells that stably express LC3-GFP and that were transfected with non-targeting, control siRNA (ntRNA) or siRNA specific for mTOR or SOD1 either in the presence of antioxidant (NAC) or absence of antioxidant (−).

FIG. 42 shows the gene symbol, Unigene ID number and name of genes for which the inhibition of their product results in enhancement of autophagy in the absence but not in the presence of antioxidant.

FIG. 43 shows the quantification of the average type III PI3 kinase activity following inhibition of the products of the autophagy-related genes of the invention able (yes) or unable (no) to induce autophagy in the presence of antioxidant (NAC).

FIG. 44 shows the gene symbol, Unigene ID number and name of genes for which the inhibition of their product results in enhancement of autophagy in the presence of antioxidant.

FIG. 45 shows an enrichment analysis of canonical pathways (MSigDB) among the hit genes relative to all genes examined in the screen. A p-value<0.05 (hyper geometric distribution) is considered significant. Only categories with at least five genes are displayed (Figure discloses ‘DEAD’ as SEQ ID NO: 1).

FIG. 46 shows that down-regulation of autophagy by 50 ng/mL FGF2 is prevented by addition of MEK inhibitor UO126. H4 cells were grown in serum-free media, levels of autophagy were assessed in the presence of 10 μg/mL E64d, with antibodies against LC3, inhibition MEK with phospho-ERK 1/2, phospho-RSK and phospho-S6 (Ser235/236). Quantification of LC3 II/tubulin ratio is shown.

FIG. 47 shows, an enrichment analysis of cis-regulatory elements/transcription factor (TF)-binding sites in the promoters of the hit genes, using motif-based gene sets from MSigDB and TF-binding sites defined in the TRANSFAC database. SRF sites are highlighted.

FIG. 48 shows a western-blot depicting the phosphorylation of Stat3 following treatment with 50 ng/mL CLCF1.

FIG. 49 shows that the down-regulation of autophagy by 50 ng/mL LIF is prevented by siRNA mediated knock-down of Stat3. H4 cells were transfected with indicated siRNAs for 72 h, than cells were treated as described for FIG. 46. Protein levels and phosphorylation of Stat3 are shown.

FIG. 50 shows that suppression of autophagy by 100 ng/mL IGF1 is prevented by Akt inhibitor VIII. Cells were treated as described for FIG. 46. Akt activity was assessed with antibodies against phospho-Foxo3a and phospho-rpS6.

FIG. 51 shows a clustering analysis of mRNA expression levels of select autophagy hit genes in young (≦40 years-old) or old (≧70 years old) human brain samples.

FIG. 52 shows a correlation matrix for the data presented in FIG. 45.

FIG. 53 shows a clustering analysis (dChip) of mRNA expression levels of select autophagy hit genes in young (≦40 years-old) or old (≧70 years old) human brain samples.

FIG. 54 shows a correlation matrix for autophagy-related genes of the invention with the most significant age-dependent regulation.

FIG. 55 shows the gene symbol, Unigene ID number, fold change and p value of autophagy-related genes of the invention that are differentially regulated in human brains during aging.

FIG. 56 shows the expression levels of autophagy-related genes of the invention during aging.

FIG. 57 shows that differential gene expression leads to up regulation of autophagy in Alzheimer's disease. Forrest plots of Normalized Enrichment Score (NES) estimates with standard deviation for the screen hit gene sets are shown. FIG. 57A shows a GSEA analysis of overall screen hit gene expression in different regions of AD brain as compared to unaffected age-matched controls. FIGS. 57B and 57C show GSEA analysis of hit genes determined to function as negative (B) or positive (C) regulators of autophagy flux. The size of a square is inversely proportional to the respective SD.

FIG. 58 shows a comparison of the levels of LC3-II accumulation in the presence or absence of 10 nM E64d following treatment of H4 cells with 5 nM Aβ.

FIG. 59 shows that Aβ induces accumulation of PtdIns3P. FYVE-dsRed cells were prepared as described in FIG. 58, fixed and imaged. Where indicated the type III PI3 kinase inhibitor 3MA (10 mM) was added for 8 hours prior to fixation.

FIG. 60 shows that the induction of the type III PI3 kinase activity by Aβ is suppressed in the presence of antioxidant. Cells were prepared as described in FIG. 59 and treated with or without antioxidant NAC.

FIG. 61 shows that the induction of autophagy by Aβ is dependent on the type III PI3 kinase activity. H4 GFP-LC3 cells were treated and imaged as described for FIG. 59.

FIG. 62 shows that the induction of autophagy by Aβ is dependent on the type III PI3 kinase activity. H4 cells were transfected with siRNA against the type III PI3 kinase subunit Vps34 or non-targeting control siRNA and than treated as described in FIG. 59. Autophagy and lysosomal changes were determined using antibodies against LC3 and Lamp 2, respectively.

FIG. 63 shows the chemical structures of select small molecule agents that modulate activity of autophagy-related genes of the invention.

FIG. 64 shows the Genbank accession numbers, names, gene symbols and mRNA sequences of the autophagy-related genes of the invention (SEQ ID NOS 11-246, respectively, in order of appearance).

### DETAILED DESCRIPTION

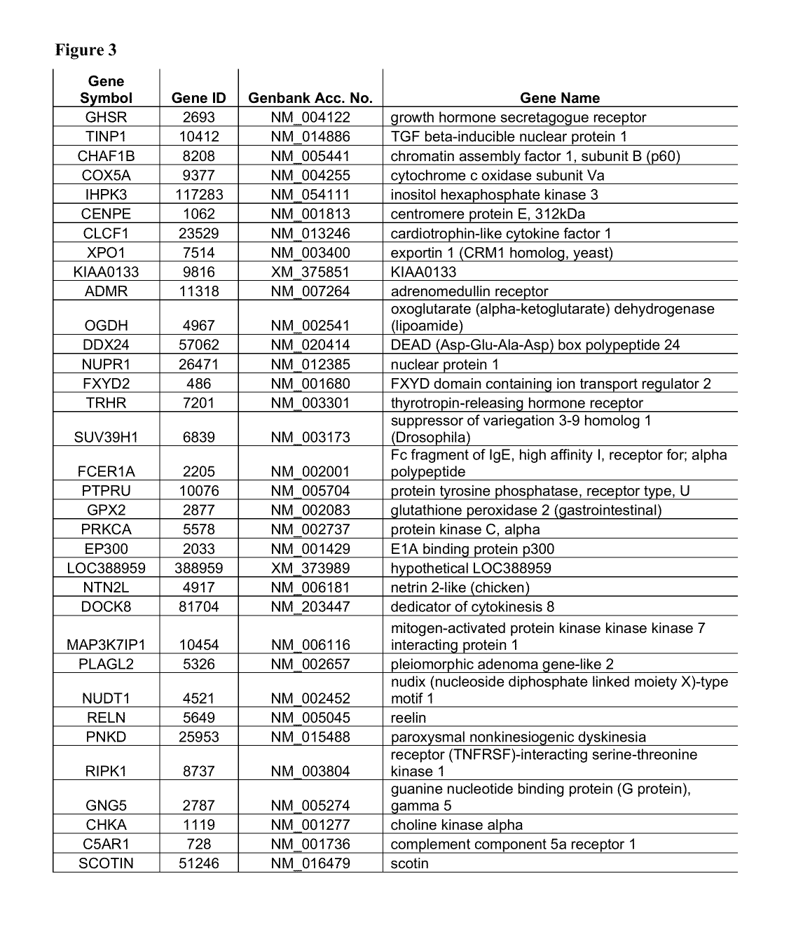

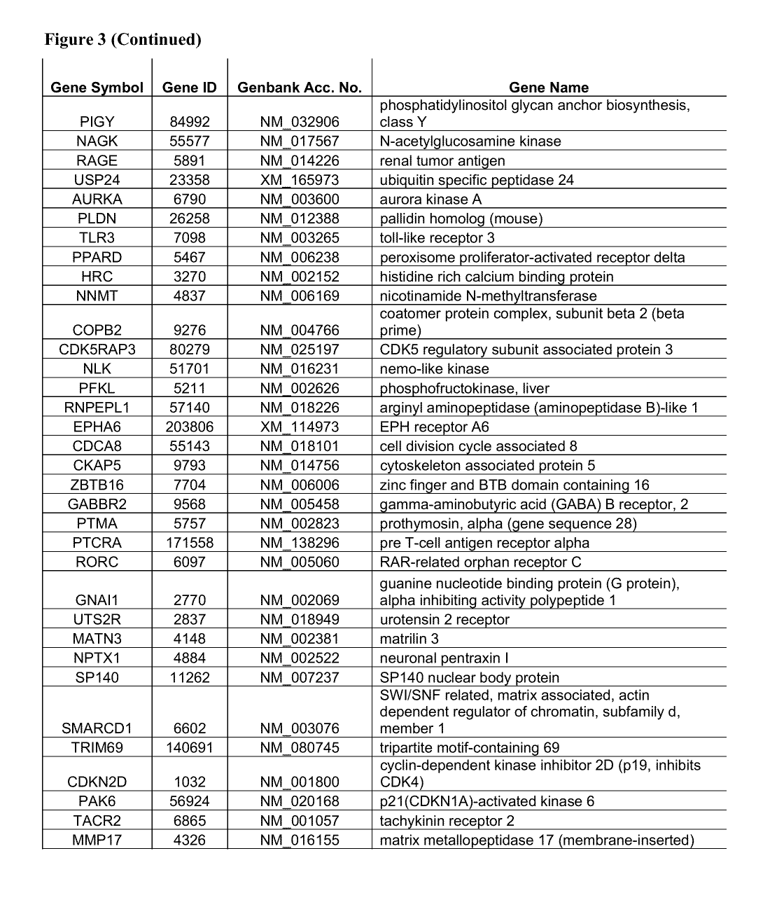

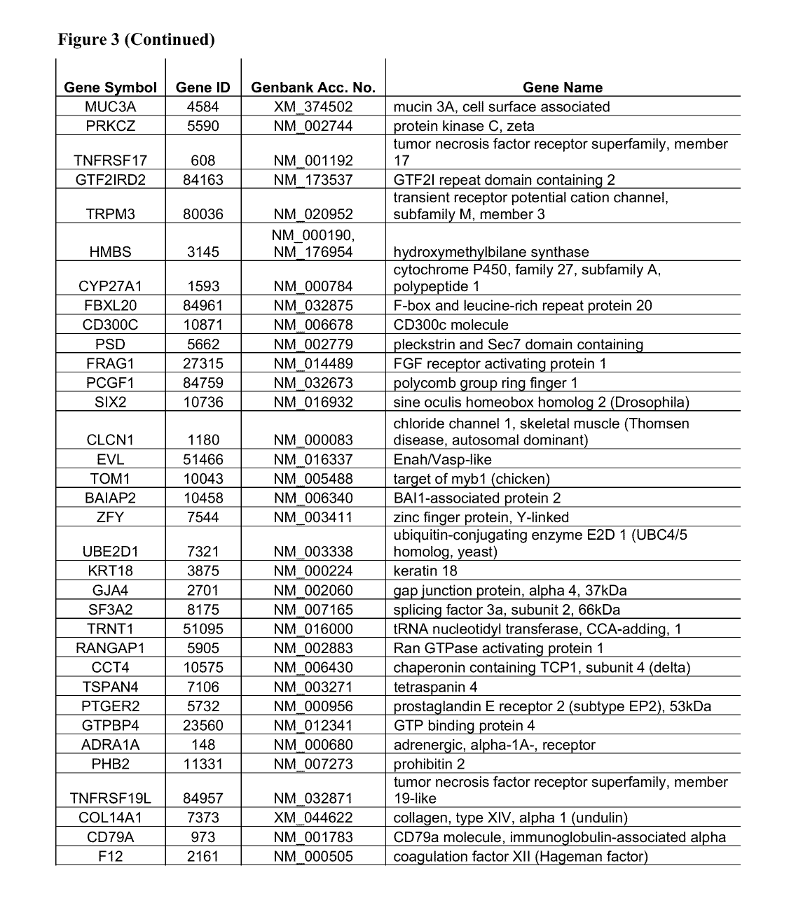

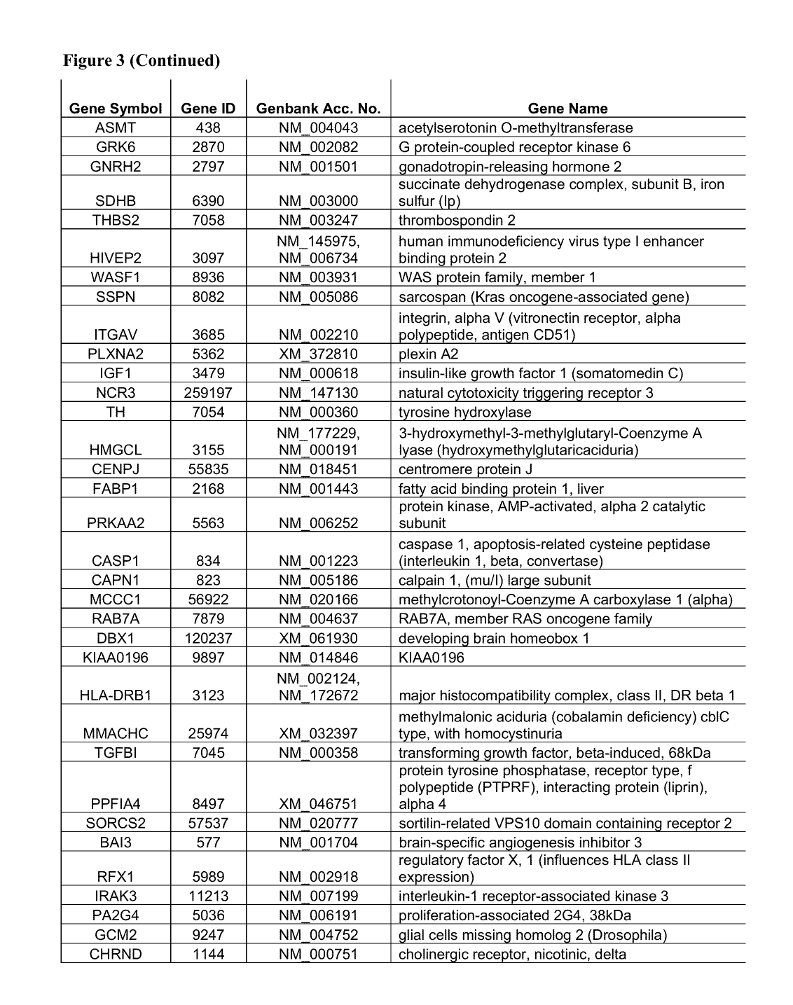

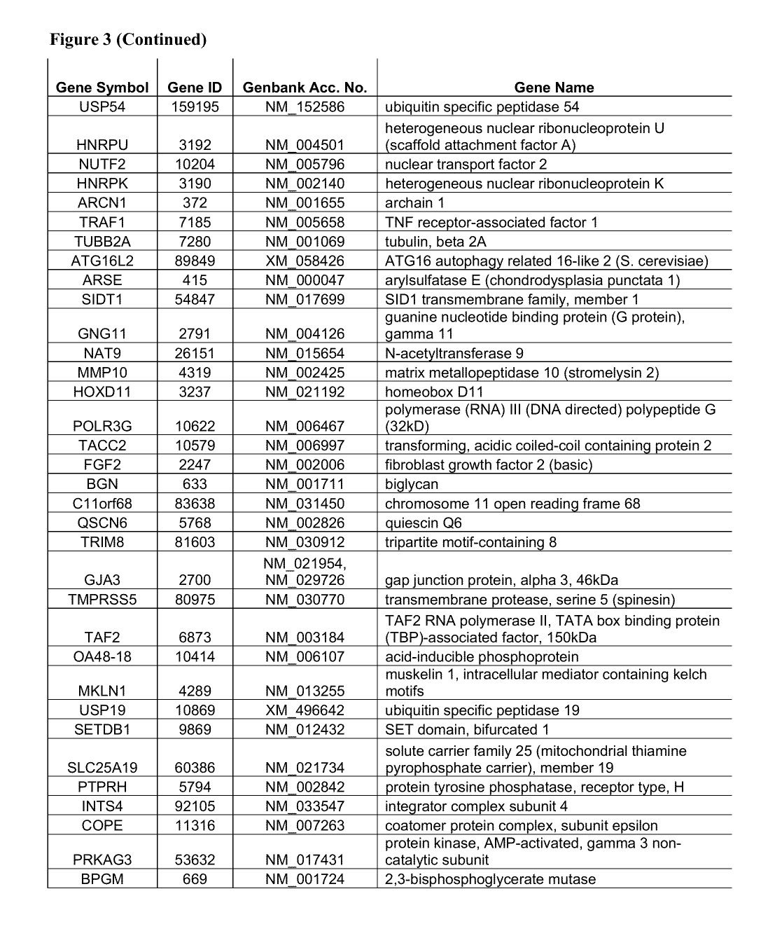

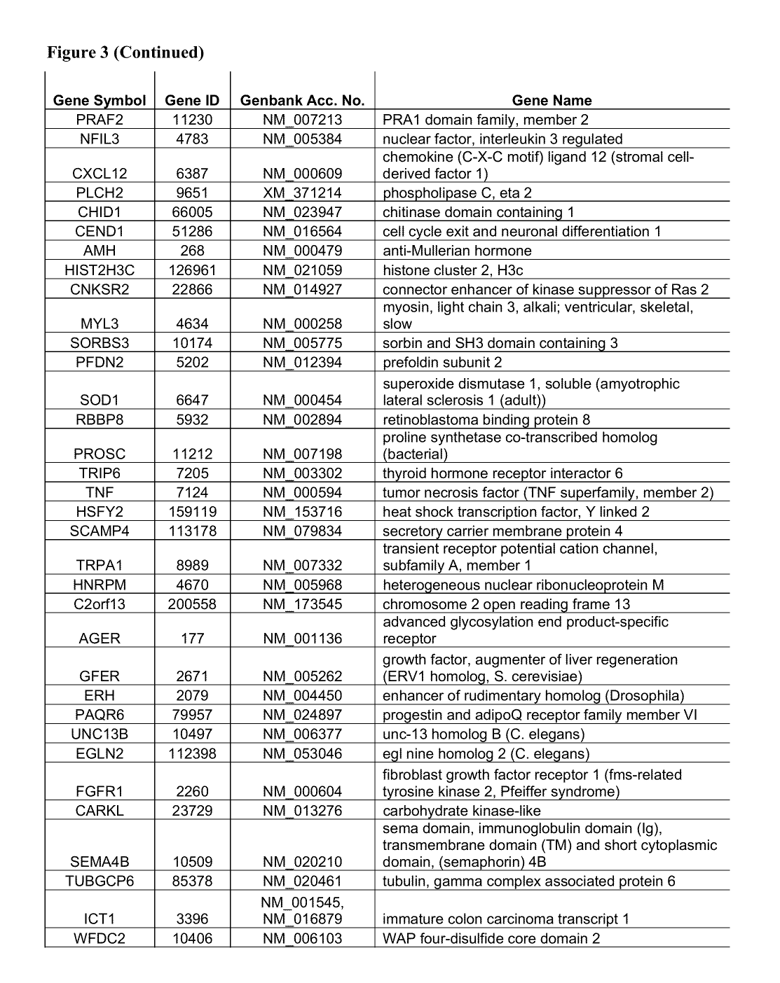

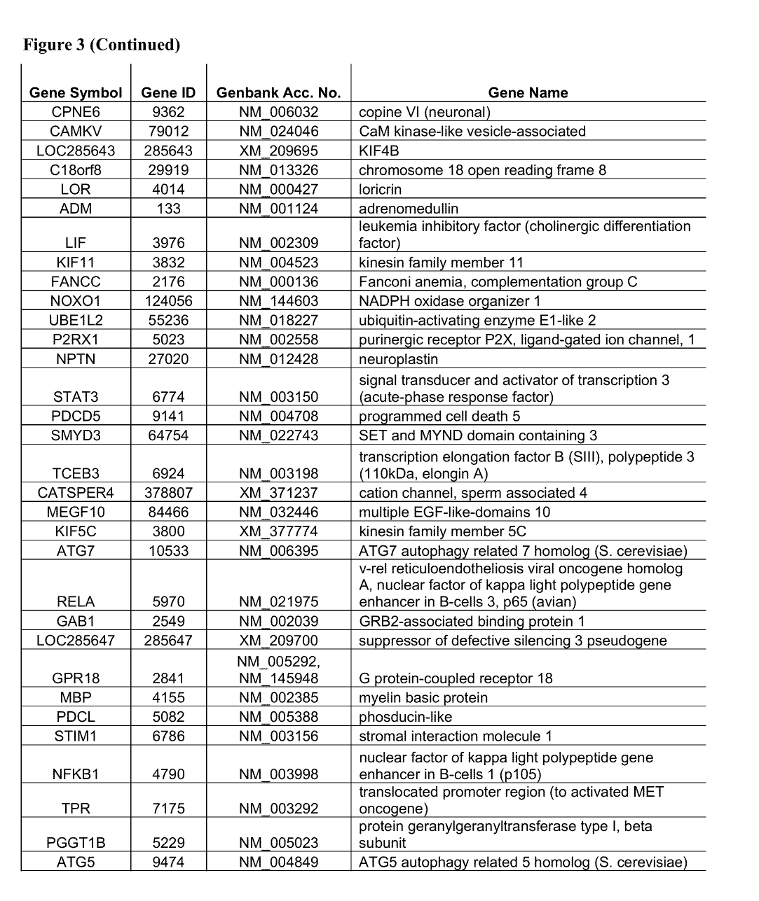

Autophagy is a lysosome-dependent catabolic process that mediates turnover of cellular components and protects multicellular eukaryotes from a wide range of diseases. In order to develop new methods for the modulation of autophagy and the treatment of autophagy-related diseases, a high-throughput image-based genome-wide screen of a human siRNA library was performed to identify genes involved in autophagy modulation and regulation. This screen led to the identification of 236 autophagy-related genes that, when knocked-down, led to either an increase or a decrease in levels of autophagy under normal nutrient conditions. The autophagy-related genes of the invention are listed in FIG. 3. These genes were extensively characterized using a combination of high-throughput assays, low-throughput assays and bioinformatics analysis. Based on the results of these studies, biological and pharmaceutical agents useful in the modulation of these genes and their gene products were identified and novel methods for the modulation of autophagy and the treatment of autophagy-related diseases were identified. The present invention, therefore, provides novel methods for the modulation of autophagy and the treatment of autophagy-related diseases, including cancer, neurodegenerative diseases, liver diseases, muscle diseases and pancreatitis.

### 1. DEFINITIONS

In order for the present invention to be more readily understood, certain terms and phrases are defined below and throughout the specification.

The articles “a” and “an” are used herein to refer to one or to more than one (i.e., to at least one) of the grammatical object of the article. By way of example, “an element” means one element or more than one element.

As used herein, the term “administering” means providing a pharmaceutical agent or composition to a subject, and includes, but is not limited to, administering by a medical professional and self-administering.

As used herein, the term “agent” refers to an entity capable of having a desired biological effect on a subject or cell. A variety of therapeutic agents is known in the art and may be identified by their effects. Examples of therapeutic agents of biological origin include growth factors, hormones, and cytokines. A variety of therapeutic agents is known in the art and may be identified by their effects. Examples include small molecules (e.g., drugs), antibodies, peptides, proteins (e.g., cytokines, hormones, soluble receptors and nonspecific-proteins), oligonucleotides (e.g., peptide-coding DNA and RNA, double-stranded RNA and antisense RNA) and peptidomimetics.

As used herein, the term “antibody” includes full-length antibodies and any antigen binding fragment (i.e., “antigen-binding portion”) or single chain thereof. The term “antibody” includes, but is not limited to, a glycoprotein comprising at least two heavy (H) chains and two light (L) chains inter-connected by disulfide bonds, or an antigen binding portion thereof. Antibodies may be polyclonal or monoclonal; xenogeneic, allogeneic, or syngeneic; or modified forms thereof (e.g., humanized, chimeric).

As used herein, the phrase “antigen-binding portion” of an antibody, refers to one or more fragments of an antibody that retain the ability to specifically bind to an antigen. The antigen-binding function of an antibody can be performed by fragments of a full-length antibody. Examples of binding fragments encompassed within the term “antigen-binding portion” of an antibody include (i) a Fab fragment, a monovalent fragment consisting of the V_{H}, V_{L}, CL and CH1 domains; (ii) a F(ab′)₂ fragment, a bivalent fragment comprising two Fab fragments linked by a disulfide bridge at the hinge region; (iii) a Fd fragment consisting of the V_{H} and CH1 domains; (iv) a Fv fragment consisting of the V_{H} and V_{L} domains of a single arm of an antibody, (v) a dAb fragment (Ward et al., (1989) Nature 341:544 546), which consists of a V_{H} domain; and (vi) an isolated complementarity determining region (CDR) or (vii) a combination of two or more isolated CDRs which may optionally be joined by a synthetic linker. Furthermore, although the two domains of the Fv fragment, V_{H} and V_{L}, are coded for by separate genes, they can be joined, using recombinant methods, by a synthetic linker that enables them to be made as a single protein chain in which the V_{H} and V_{L} regions pair to form monovalent molecules (known as single chain Fv (scFv); see e.g., Bird et al. (1988) Science 242:423 426; and Huston et al. (1988) Proc. Natl. Acad. Sci. USA 85:5879 5883). Such single chain antibodies are also intended to be encompassed within the term “antigen-binding portion” of an antibody. These antibody fragments are obtained using conventional techniques known to those with skill in the art, and the fragments are screened for utility in the same manner as are intact antibodies.

As used herein, the term “cancer” includes, but is not limited to, solid tumors and blood borne tumors. The term cancer includes diseases of the skin, tissues, organs, bone, cartilage, blood and vessels. The term “cancer” further encompasses both primary and metastatic cancers.

As used herein, the phrases “gene product” and “product of a gene” refers to a substance encoded by a gene and able to be produced, either directly or indirectly, through the transcription of the gene. The phrases “gene product” and “product of a gene” include RNA gene products (e.g. mRNA), DNA gene products (e.g. cDNA) and polypeptide gene products (e.g. proteins).

As used herein, the phrase “enhancing the activity” of a gene product refers to an increase in a particular activity associated with the gene product. Examples of enhanced activity include, but are not limited to, increased translation of mRNA, increased signal transduction by polypeptides or proteins and increased catalysis by enzymes. Enhancement of activity can occur, for example, through an increased amount of activity performed by individual gene products, through an increase number of gene products performing the activity, or a through any combination thereof. If a gene product enhances a biological process (e.g. autophagy), “enhancing the activity” of such a gene product will generally enhance the process. Conversely, if a gene product functions as an inhibitor of a biological process, “enhancing the activity” of such a gene product will generally inhibit the process.

As used herein, the phrase “inhibiting the activity” of a gene product refers to a decrease in a particular activity associated with the gene product. Examples of inhibited activity include, but are not limited to, decreased translation of mRNA, decreased signal transduction by polypeptides or proteins and decreased catalysis by enzymes. Inhibition of activity can occur, for example, through a reduced amount of activity performed by individual gene products, through a decreased number of gene products performing the activity, or a through any combination thereof. If a gene product enhances a biological process (e.g. autophagy), “inhibiting the activity” of such a gene product will generally inhibit the process. Conversely, if a gene product functions as an inhibitor of a biological process, “inhibiting the activity” of such a gene product will generally enhance the process.

As used herein, the term “isolated” refers to the state in which substances (e.g., polypeptides or polynucleotides) are free or substantially free of material with which they are naturally associated such as other polypeptides or polynucleotides with which they are found in their natural environment or the environment in which they are prepared (e.g., cell culture). Polypeptides or polynucleotides can be formulated with diluents or adjuvants and still be considered “isolated”—for example, polypeptides or polynucleotides can be mixed with pharmaceutically acceptable carriers or diluents when used in diagnosis or therapy.

As used herein, the term “modulation” refers to up regulation (i.e., activation or stimulation), down regulation (i e, inhibition or suppression) of a biological activity, or the two in combination or apart.

As used herein, the phrases “neurodegenerative disorder” and “neurodegenerative disease” refers to a wide range of diseases and/or disorders of the central and peripheral nervous system, such as neuropathologies, and includes but is not limited to, Parkinson's disease, Alzheimer's disease (AD), amyotrophic lateral sclerosis (ALS), denervation atrophy, otosclerosis, stroke, dementia, multiple sclerosis, Huntington's disease, encephalopathy associated with acquired immunodeficiency disease (AIDS), and other diseases associated with neuronal cell toxicity and cell death.

As used herein, the phrase “pharmaceutically acceptable” refers to those agents, compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the phrase “pharmaceutically-acceptable carrier” means a pharmaceutically-acceptable material, composition or vehicle, such as a liquid or solid filler, diluent, excipient, or solvent encapsulating material, involved in carrying or transporting an agent from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; and (22) other non-toxic compatible substances employed in pharmaceutical formulations.

As used herein, the phrase “pharmaceutically-acceptable salts” refers to the relatively non-toxic, inorganic and organic salts of compounds.

As used herein, the term “subject” means a human or non-human animal selected for treatment or therapy.

As used herein, the phrase “subject suspected of having” means a subject exhibiting one or more clinical indicators of a disease or condition. In certain embodiments, the disease or condition is cancer, a neurodegenerative disorder or pancreatitis.

As used herein, the phrase “subject in need thereof” means a subject identified as in need of a therapy or treatment of the invention.

As used herein, the phrase “therapeutic effect” refers to a local or systemic effect in animals, particularly mammals, and more particularly humans, caused by an agent. The phrases “therapeutically-effective amount” and “effective amount” mean the amount of an agent that produces some desired effect in at least a sub-population of cells. A therapeutically effective amount includes an amount of an agent that produces some desired local or systemic effect at a reasonable benefit/risk ratio applicable to any treatment. For example, certain agents used in the methods of the present invention may be administered in a sufficient amount to produce a reasonable benefit/risk ratio applicable to such treatment.

As used herein, the term “treating” a disease in a subject or “treating” a subject having or suspected of having a disease refers to subjecting the subject to a pharmaceutical treatment, e.g., the administration of an agent, such that at least one symptom of the disease is decreased or prevented from worsening.

### 2. AUTOPHAGY-RELATED GENES

The autophagy-related genes of the present invention can be divided into genes whose products inhibit autophagy (or autophagy-inhibiting genes, listed in Table 1) and genes whose products enhance autophagy (or autophagy-enhancing genes, listed in Table 2).

Agents that modulate the activity of products of autophagy-inhibiting genes are useful in the treatment of autophagy-related diseases. Agents that inhibit the activity of the products of autophagy-inhibiting genes result in elevated autophagy levels and are therefore useful in methods of enhancing autophagy and the treatment of autophagy-related diseases that are responsive to elevated levels of autophagy, such as neurodegenerative diseases and proteinopathies. On the other hand, agents that enhance the activity of products of autophagy-inhibiting genes result in reduced autophagy levels, and are therefore useful in methods of inhibition of autophagy and the treatment of autophagy-related diseases that are responsive to autophagy inhibition, such as cancer and pancreatitis.

TABLE 1
Autophagy-inhibiting genes.

| Gene | | | |
|---|---|---|---|
| Symbol | Gene ID | Genbank Acc. No. | Gene Name |
| GHSR | 2693 | NM_004122 | growth hormone secretagogue receptor |
| TINP1 | 10412 | NM_014886 | TGF beta-inducible nuclear protein 1 |
| CHAF1B | 8208 | NM_005441 | chromatin assembly factor 1, subunit B (p60) |
| COX5A | 9377 | NM_004255 | cytochrome c oxidase subunit Va |
| IHPK3 | 117283 | NM_054111 | inositol hexaphosphate kinase 3 |
| CENPE | 1062 | NM_001813 | centromere protein E, 312 kDa |
| CLCF1 | 23529 | NM_013246 | cardiotrophin-like cytokine factor 1 |
| XPO1 | 7514 | NM_003400 | exportin 1 (CRM1 homolog, yeast) |
| KIAA0133 | 9816 | XM_375851 | KIAA0133 |
| ADMR | 11318 | NM_007264 | adrenomedullin receptor |
| OGDH | 4967 | NM_002541 | oxoglutarate (alpha-ketoglutarate) dehydrogenase |
| | | | (lipoamide) |
| DDX24 | 57062 | NM_020414 | DEAD (Asp-Glu-Ala-Asp, SEQ ID NO: 1) box |
| | | | polypeptide 24 |
| NUPR1 | 26471 | NM_012385 | nuclear protein 1 |
| FXYD2 | 486 | NM_001680 | FXYD domain containing ion transport regulator 2 |
| TRHR | 7201 | NM_003301 | thyrotropin-releasing hormone receptor |
| SUV39H1 | 6839 | NM_003173 | suppressor of variegation 3-9 homolog 1 |
| | | | (Drosophila) |
| FCER1A | 2205 | NM_002001 | Fc fragment of IgE, high affinity I, receptor for; alpha |
| | | | polypeptide |
| PTPRU | 10076 | NM_005704 | protein tyrosine phosphatase, receptor type, U |
| GPX2 | 2877 | NM_002083 | glutathione peroxidase 2 (gastrointestinal) |
| PRKCA | 5578 | NM_002737 | protein kinase C, alpha |
| EP300 | 2033 | NM_001429 | E1A binding protein p300 |
| LOC388959 | 388959 | XM_373989 | hypothetical LOC388959 |
| NTN2L | 4917 | NM_006181 | netrin 2-like (chicken) |
| DOCK8 | 81704 | NM_203447 | dedicator of cytokinesis 8 |
| MAP3K7IP1 | 10454 | NM_006116 | mitogen-activated protein kinase kinase kinase 7 |
| | | | interacting protein 1 |
| PLAGL2 | 5326 | NM_002657 | pleiomorphic adenoma gene-like 2 |
| NUDT1 | 4521 | NM_002452 | nudix (nucleoside diphosphate linked moiety X)- |
| | | | type motif 1 |
| RELN | 5649 | NM_005045 | reelin |
| PNKD | 25953 | NM_015488 | paroxysmal nonkinesiogenic dyskinesia |
| RIPK1 | 8737 | NM_003804 | receptor (TNFRSF)-interacting serine-threonine |
| | | | kinase 1 |
| GNG5 | 2787 | NM_005274 | guanine nucleotide binding protein (G protein), |
| | | | gamma 5 |
| CHKA | 1119 | NM_001277 | choline kinase alpha |
| C5AR1 | 728 | NM_001736 | complement component 5a receptor 1 |
| SCOTIN | 51246 | NM_016479 | scotin |
| PIGY | 84992 | NM_032906 | phosphatidylinositol glycan anchor biosynthesis, |
| | | | class Y |
| NAGK | 55577 | NM_017567 | N-acetylglucosamine kinase |
| RAGE | 5891 | NM_014226 | renal tumor antigen |
| USP24 | 23358 | XM_165973 | ubiquitin specific peptidase 24 |
| AURKA | 6790 | NM_003600 | aurora kinase A |
| PLDN | 26258 | NM_012388 | pallidin homolog (mouse) |
| TLR3 | 7098 | NM_003265 | toll-like receptor 3 |
| PPARD | 5467 | NM_006238 | peroxisome proliferator-activated receptor delta |
| HRC | 3270 | NM_002152 | histidine rich calcium binding protein |
| NNMT | 4837 | NM_006169 | nicotinamide N-methyltransferase |
| COPB2 | 9276 | NM_004766 | coatomer protein complex, subunit beta 2 (beta |
| | | | prime) |
| CDK5RAP3 | 80279 | NM_025197 | CDK5 regulatory subunit associated protein 3 |
| NLK | 51701 | NM_016231 | nemo-like kinase |
| PFKL | 5211 | NM_002626 | phosphofructokinase, liver |
| RNPEPL1 | 57140 | NM_018226 | arginyl aminopeptidase (aminopeptidase B)-like 1 |
| EPHA6 | 203806 | XM_114973 | EPH receptor A6 |
| CDCA8 | 55143 | NM_018101 | cell division cycle associated 8 |
| CKAP5 | 9793 | NM_014756 | cytoskeleton associated protein 5 |
| ZBTB16 | 7704 | NM_006006 | zinc finger and BTB domain containing 16 |
| GABBR2 | 9568 | NM_005458 | gamma-aminobutyric acid (GABA) B receptor, 2 |
| PTMA | 5757 | NM_002823 | prothymosin, alpha (gene sequence 28) |
| PTCRA | 171558 | NM_138296 | pre T-cell antigen receptor alpha |
| RORC | 6097 | NM_005060 | RAR-related orphan receptor C |
| GNAI1 | 2770 | NM_002069 | guanine nucleotide binding protein (G protein), |
| | | | alpha inhibiting activity polypeptide 1 |
| UTS2R | 2837 | NM_018949 | urotensin 2 receptor |
| MATN3 | 4148 | NM_002381 | matrilin 3 |
| NPTX1 | 4884 | NM_002522 | neuronal pentraxin I |
| SP140 | 11262 | NM_007237 | SP140 nuclear body protein |
| SMARCD1 | 6602 | NM_003076 | SWI/SNF related, matrix associated, actin |
| | | | dependent regulator of chromatin, subfamily d, |
| | | | member 1 |
| TRIM69 | 140691 | NM_080745 | tripartite motif-containing 69 |
| CDKN2D | 1032 | NM_001800 | cyclin-dependent kinase inhibitor 2D (p19, inhibits |
| | | | CDK4) |
| PAK6 | 56924 | NM_020168 | p21(CDKN1A)-activated kinase 6 |
| TACR2 | 6865 | NM_001057 | tachykinin receptor 2 |
| MMP17 | 4326 | NM_016155 | matrix metallopeptidase 17 (membrane-inserted) |
| MUC3A | 4584 | XM_374502 | mucin 3A, cell surface associated |
| PRKCZ | 5590 | NM_002744 | protein kinase C, zeta |
| TNFRSF17 | 608 | NM_001192 | tumor necrosis factor receptor superfamily, member |
| | | | 17 |
| GTF2IRD2 | 84163 | NM_173537 | GTF2I repeat domain containing 2 |
| TRPM3 | 80036 | NM_020952 | transient receptor potential cation channel, |
| | | | subfamily M, member 3 |
| HMBS | 3145 | NM_000190, | hydroxymethylbilane synthase |
| | | NM_176954 | |
| CYP27A1 | 1593 | NM_000784 | cytochrome P450, family 27, subfamily A, |
| | | | polypeptide 1 |
| FBXL20 | 84961 | NM_032875 | F-box and leucine-rich repeat protein 20 |
| CD300C | 10871 | NM_006678 | CD300c molecule |
| PSD | 5662 | NM_002779 | pleckstrin and Sec7 domain containing |
| FRAG1 | 27315 | NM_014489 | FGF receptor activating protein 1 |
| PCGF1 | 84759 | NM_032673 | polycomb group ring finger 1 |
| SIX2 | 10736 | NM_016932 | sine oculis homeobox homolog 2 (Drosophila) |
| CLCN1 | 1180 | NM_000083 | chloride channel 1, skeletal muscle (Thomsen |
| | | | disease, autosomal dominant) |
| EVL | 51466 | NM_016337 | Enah/Vasp-like |
| TOM1 | 10043 | NM_005488 | target of myb1 (chicken) |
| BAIAP2 | 10458 | NM_006340 | BAI1-associated protein 2 |
| ZFY | 7544 | NM_003411 | zinc finger protein, Y-linked |
| UBE2D1 | 7321 | NM_003338 | ubiquitin-conjugating enzyme E2D 1 (UBC4/5 |
| | | | homolog, yeast) |
| KRT18 | 3875 | NM_000224 | keratin 18 |
| GJA4 | 2701 | NM_002060 | gap junction protein, alpha 4, 37 kDa |
| SF3A2 | 8175 | NM_007165 | splicing factor 3a, subunit 2, 66 kDa |
| TRNT1 | 51095 | NM_016000 | tRNA nucleotidyl transferase, CCA-adding, 1 |
| RANGAP1 | 5905 | NM_002883 | Ran GTPase activating protein 1 |
| CCT4 | 10575 | NM_006430 | chaperonin containing TCP1, subunit 4 (delta) |
| TSPAN4 | 7106 | NM_003271 | tetraspanin 4 |
| PTGER2 | 5732 | NM_000956 | prostaglandin E receptor 2 (subtype EP2), 53 kDa |
| GTPBP4 | 23560 | NM_012341 | GTP binding protein 4 |
| ADRA1A | 148 | NM_000680 | adrenergic, alpha-1A-, receptor |
| PHB2 | 11331 | NM_007273 | prohibitin 2 |
| TNFRSF19L | 84957 | NM_032871 | tumor necrosis factor receptor superfamily, member |
| | | | 19-like |
| COL14A1 | 7373 | XM_044622 | collagen, type XIV, alpha 1 (undulin) |
| CD79A | 973 | NM_001783 | CD79a molecule, immunoglobulin-associated alpha |
| F12 | 2161 | NM_000505 | coagulation factor XII (Hageman factor) |
| ASMT | 438 | NM_004043 | acetylserotonin O-methyltransferase |
| GRK6 | 2870 | NM_002082 | G protein-coupled receptor kinase 6 |
| GNRH2 | 2797 | NM_001501 | gonadotropin-releasing hormone 2 |
| SDHB | 6390 | NM_003000 | succinate dehydrogenase complex, subunit B, iron |
| | | | sulfur (Ip) |
| THBS2 | 7058 | NM_003247 | thrombospondin 2 |
| HIVEP2 | 3097 | NM_145975, | human immunodeficiency virus type I enhancer |
| | | NM_006734 | binding protein 2 |
| WASF1 | 8936 | NM_003931 | WAS protein family, member 1 |
| SSPN | 8082 | NM_005086 | sarcospan (Kras oncogene-associated gene) |
| ITGAV | 3685 | NM_002210 | integrin, alpha V (vitronectin receptor, alpha |
| | | | polypeptide, antigen CD51) |
| PLXNA2 | 5362 | XM_372810 | plexin A2 |
| IGF1 | 3479 | NM_000618 | insulin-like growth factor 1 (somatomedin C) |
| NCR3 | 259197 | NM_147130 | natural cytotoxicity triggering receptor 3 |
| TH | 7054 | NM_000360 | tyrosine hydroxylase |
| HMGCL | 3155 | NM_177229, | 3-hydroxymethyl-3-methylglutaryl-Coenzyme A |
| | | NM_000191 | lyase (hydroxymethylglutaricaciduria) |
| CENPJ | 55835 | NM_018451 | centromere protein J |
| FABP1 | 2168 | NM_001443 | fatty acid binding protein 1, liver |
| PRKAA2 | 5563 | NM_006252 | protein kinase, AMP-activated, alpha 2 catalytic |
| | | | subunit |
| CASP1 | 834 | NM_001223 | caspase 1, apoptosis-related cysteine peptidase |
| | | | (interleukin 1, beta, convertase) |
| CAPN1 | 823 | NM_005186 | calpain 1, (mu/l) large subunit |
| MCCC1 | 56922 | NM_020166 | methylcrotonoyl-Coenzyme A carboxylase 1 (alpha) |
| RAB7A | 7879 | NM_004637 | RAB7A, member RAS oncogene family |
| DBX1 | 120237 | XM_061930 | developing brain homeobox 1 |
| KIAA0196 | 9897 | NM_014846 | KIAA0196 |
| HLA-DRB1 | 3123 | NM_002124, | major histocompatibility complex, class II, DR beta 1 |
| | | NM_172672 | |
| MMACHC | 25974 | XM_032397 | methylmalonic aciduria (cobalamin deficiency) cblC |
| | | | type, with homocystinuria |
| TGFBI | 7045 | NM_000358 | transforming growth factor, beta-induced, 68 kDa |
| PPFIA4 | 8497 | XM_046751 | protein tyrosine phosphatase, receptor type, f |
| | | | polypeptide (PTPRF), interacting protein (liprin), |
| | | | alpha 4 |
| SORCS2 | 57537 | NM_020777 | sortilin-related VPS10 domain containing receptor 2 |
| BAI3 | 577 | NM_001704 | brain-specific angiogenesis inhibitor 3 |
| RFX1 | 5989 | NM_002918 | regulatory factor X, 1 (influences HLA class II |
| | | | expression) |
| IRAK3 | 11213 | NM_007199 | interleukin-1 receptor-associated kinase 3 |
| PA2G4 | 5036 | NM_006191 | proliferation-associated 2G4, 38 kDa |
| GCM2 | 9247 | NM_004752 | glial cells missing homolog 2 (Drosophila) |
| CHRND | 1144 | NM_000751 | cholinergic receptor, nicotinic, delta |
| USP54 | 159195 | NM_152586 | ubiquitin specific peptidase 54 |
| HNRPU | 3192 | NM_004501 | heterogeneous nuclear ribonucleoprotein U |
| | | | (scaffold attachment factor A) |
| NUTF2 | 10204 | NM_005796 | nuclear transport factor 2 |
| HNRPK | 3190 | NM_002140 | heterogeneous nuclear ribonucleoprotein K |
| ARCN1 | 372 | NM_001655 | archain 1 |
| TRAF1 | 7185 | NM_005658 | TNF receptor-associated factor 1 |
| TUBB2A | 7280 | NM_001069 | tubulin, beta 2A |
| ATG16L2 | 89849 | XM_058426 | ATG16 autophagy related 16-like 2 (S. cerevisiae) |
| ARSE | 415 | NM_000047 | arylsulfatase E (chondrodysplasia punctata 1) |
| SIDT1 | 54847 | NM_017699 | SID1 transmembrane family, member 1 |
| GNG11 | 2791 | NM_004126 | guanine nucleotide binding protein (G protein), |
| | | | gamma 11 |
| NAT9 | 26151 | NM_015654 | N-acetyltransferase 9 |
| MMP10 | 4319 | NM_002425 | matrix metallopeptidase 10 (stromelysin 2) |
| HOXD11 | 3237 | NM_021192 | homeobox D11 |
| POLR3G | 10622 | NM_006467 | polymerase (RNA) III (DNA directed) polypeptide G |
| | | | (32 kD) |
| TACC2 | 10579 | NM_006997 | transforming, acidic coiled-coil containing protein 2 |
| FGF2 | 2247 | NM_002006 | fibroblast growth factor 2 (basic) |
| BGN | 633 | NM_001711 | biglycan |
| C11orf68 | 83638 | NM_031450 | chromosome 11 open reading frame 68 |
| QSCN6 | 5768 | NM_002826 | quiescin Q6 |
| TRIM8 | 81603 | NM_030912 | tripartite motif-containing 8 |
| GJA3 | 2700 | NM_021954, | gap junction protein, alpha 3, 46 kDa |
| | | NM_029726 | |
| TMPRSS5 | 80975 | NM_030770 | transmembrane protease, serine 5 (spinesin) |
| TAF2 | 6873 | NM_003184 | TAF2 RNA polymerase II, TATA box binding protein |
| | | | (TBP)-associated factor, 150 kDa |
| OA48-18 | 10414 | NM_006107 | acid-inducible phosphoprotein |
| MKLN1 | 4289 | NM_013255 | muskelin 1, intracellular mediator containing kelch |
| | | | motifs |
| USP19 | 10869 | XM_496642 | ubiquitin specific peptidase 19 |
| SETDB1 | 9869 | NM_012432 | SET domain, bifurcated 1 |
| SLC25A19 | 60386 | NM_021734 | solute carrier family 25 (mitochondrial thiamine |
| | | | pyrophosphate carrier), member 19 |
| PTPRH | 5794 | NM_002842 | protein tyrosine phosphatase, receptor type, H |
| INTS4 | 92105 | NM_033547 | integrator complex subunit 4 |
| COPE | 11316 | NM_007263 | coatomer protein complex, subunit epsilon |
| PRKAG3 | 53632 | NM_017431 | protein kinase, AMP-activated, gamma 3 non- |
| | | | catalytic subunit |
| BPGM | 669 | NM_001724 | 2,3-bisphosphoglycerate mutase |
| PRAF2 | 11230 | NM_007213 | PRA1 domain family, member 2 |
| NFIL3 | 4783 | NM_005384 | nuclear factor, interleukin 3 regulated |
| CXCL12 | 6387 | NM_000609 | chemokine (C—X—C motif) ligand 12 (stromal cell- |
| | | | derived factor 1) |
| PLCH2 | 9651 | XM_371214 | phospholipase C, eta 2 |
| CHID1 | 66005 | NM_023947 | chitinase domain containing 1 |
| CEND1 | 51286 | NM_016564 | cell cycle exit and neuronal differentiation 1 |
| AMH | 268 | NM_000479 | anti-Mullerian hormone |
| HIST2H3C | 126961 | NM_021059 | histone cluster 2, H3c |
| CNKSR2 | 22866 | NM_014927 | connector enhancer of kinase suppressor of Ras 2 |
| MYL3 | 4634 | NM_000258 | myosin, light chain 3, alkali; ventricular, skeletal, |
| | | | slow |
| SORBS3 | 10174 | NM_005775 | sorbin and SH3 domain containing 3 |
| PFDN2 | 5202 | NM_012394 | prefoldin subunit 2 |
| SOD1 | 6647 | NM_000454 | superoxide dismutase 1, soluble (amyotrophic |
| | | | lateral sclerosis 1 (adult)) |
| RBBP8 | 5932 | NM_002894 | retinoblastoma binding protein 8 |
| PROSC | 11212 | NM_007198 | proline synthetase co-transcribed homolog |
| | | | (bacterial) |
| TRIP6 | 7205 | NM_003302 | thyroid hormone receptor interactor 6 |
| TNF | 7124 | NM_000594 | tumor necrosis factor (TNF superfamily, member 2) |
| HSFY2 | 159119 | NM_153716 | heat shock transcription factor, Y linked 2 |
| SCAMP4 | 113178 | NM_079834 | secretory carrier membrane protein 4 |
| TRPA1 | 8989 | NM_007332 | transient receptor potential cation channel, |
| | | | subfamily A, member 1 |
| HNRPM | 4670 | NM_005968 | heterogeneous nuclear ribonucleoprotein M |
| C2orf13 | 200558 | NM_173545 | chromosome 2 open reading frame 13 |
| AGER | 177 | NM_001136 | advanced glycosylation end product-specific |
| | | | receptor |
| GFER | 2671 | NM_005262 | growth factor, augmenter of liver regeneration |
| | | | (ERV1 homolog, S. cerevisiae) |
| ERH | 2079 | NM_004450 | enhancer of rudimentary homolog (Drosophila) |
| PAQR6 | 79957 | NM_024897 | progestin and adipoQ receptor family member VI |
| UNC13B | 10497 | NM_006377 | unc-13 homolog B (C. elegans) |
| EGLN2 | 112398 | NM_053046 | egl nine homolog 2 (C. elegans) |
| FGFR1 | 2260 | NM_000604 | fibroblast growth factor receptor 1 (fms-related |
| | | | tyrosine kinase 2, Pfeiffer syndrome) |
| CARKL | 23729 | NM_013276 | carbohydrate kinase-like |
| SEMA4B | 10509 | NM_020210 | sema domain, immunoglobulin domain (Ig), |
| | | | transmembrane domain (TM) and short cytoplasmic |
| | | | domain, (semaphorin) 4B |
| TUBGCP6 | 85378 | NM_020461 | tubulin, gamma complex associated protein 6 |
| ICT1 | 3396 | NM_001545, | immature colon carcinoma transcript 1 |
| | | NM_016879 | |
| WFDC2 | 10406 | NM_006103 | WAP four-disulfide core domain 2 |
| CPNE6 | 9362 | NM_006032 | copine VI (neuronal) |
| CAMKV | 79012 | NM_024046 | CaM kinase-like vesicle-associated |
| LOC285643 | 285643 | XM_209695 | KIF4B |
| C18orf8 | 29919 | NM_013326 | chromosome 18 open reading frame 8 |
| LOR | 4014 | NM_000427 | loricrin |
| ADM | 133 | NM_001124 | adrenomedullin |
| LIF | 3976 | NM_002309 | leukemia inhibitory factor (cholinergic differentiation |
| | | | factor) |
| KIF11 | 3832 | NM_004523 | kinesin family member 11 |
| FANCC | 2176 | NM_000136 | Fanconi anemia, complementation group C |
| NOXO1 | 124056 | NM_144603 | NADPH oxidase organizer 1 |
| UBE1L2 | 55236 | NM_018227 | ubiquitin-activating enzyme E1-like 2 |
| P2RX1 | 5023 | NM_002558 | purinergic receptor P2X, ligand-gated ion channel, 1 |
| NPTN | 27020 | NM_012428 | neuroplastin |
| STAT3 | 6774 | NM_003150 | signal transducer and activator of transcription 3 |
| | | | (acute-phase response factor) |
| PDCD5 | 9141 | NM_004708 | programmed cell death 5 |

Agents that modulate the activity of products of autophagy-enhancing genes are also useful in the treatment of autophagy-related diseases. For example, agents that inhibit the activity of products of autophagy-enhancing genes result in reduced autophagy levels and are therefore useful in methods of inhibition of autophagy and the treatment of autophagy-related diseases that are responsive to autophagy inhibition, such as cancer and pancreatitis. Agents that enhance the activity of products of autophagy-enhancing genes result in elevated autophagy levels and are therefore useful in methods of enhancement of autophagy and the treatment of autophagy-related diseases that are responsive to elevated levels of autophagy, such as neurodegenerative diseases and proteinopathies.

TABLE 2
Autophagy-enhancing genes.

| Gene | | Genbank Acc. | |
|---|---|---|---|
| Symbol | Gene ID | No. | Gene Name |
| SMYD3 | 64754 | NM_022743 | SET and MYND domain |
| | | | containing 3 |
| TCEB3 | 6924 | NM_003198 | transcription elongation |
| | | | factor B (SIII), |
| | | | polypeptide 3 |
| | | | (110 kDa, elongin A) |
| CATSPER4 | 378807 | XM_371237 | cation channel, sperm |
| | | | associated 4 |
| MEGF10 | 84466 | NM_032446 | multiple EGF-like-domains 10 |
| KIF5C | 3800 | XM_377774 | kinesin family member 5C |
| ATG7 | 10533 | NM_006395 | ATG7 autophagy related 7 |
| | | | homolog (S. cerevisiae) |
| RELA | 5970 | NM_021975 | v-rel reticuloendotheliosis |
| | | | viral oncogene homolog A, |
| | | | nuclear factor of kappa light |
| | | | polypeptide gene enhancer in |
| | | | B-cells 3, p65 (avian) |
| GAB1 | 2549 | NM_002039 | GRB2-associated binding |
| | | | protein 1 |
| LOC285647 | 285647 | XM_209700 | suppressor of defective |
| | | | silencing 3 pseudogene |
| GPR18 | 2841 | NM_005292, | G protein-coupled receptor 18 |
| | | NM_145948 | |
| MBP | 4155 | NM_002385 | myelin basic protein |
| PDCL | 5082 | NM_005388 | phosducin-like |
| STIM1 | 6786 | NM_003156 | stromal interaction molecule 1 |
| NFKB1 | 4790 | NM_003998 | nuclear factor of kappa light |
| | | | polypeptide gene enhancer |
| | | | in B-cells 1 (p105) |
| TPR | 7175 | NM_003292 | translocated promoter |
| | | | region (to activated MET |
| | | | oncogene) |
| PGGT1B | 5229 | NM_005023 | protein |
| | | | geranylgeranyltransferase |
| | | | type I, beta subunit |
| ATG5 | 9474 | NM_004849 | ATG5 autophagy related 5 |
| | | | homolog (S. cerevisiae) |

Thus, certain embodiments of the present invention relate to methods of enhancing autophagy and/or treating neurodegenerative diseases and/or proteinopathies through the inhibition of the activity of products of the autophagy-inhibiting genes listed in Table 1 or the enhancement of the activity of products of the autophagy-enhancing genes listed in Table 2. Other embodiments of the present invention relate to methods of inhibiting autophagy and/or treating cancer or pancreatitis through the enhancement of the activity of products of the autophagy-inhibiting genes listed in Table 1 or the inhibition of the activity of products of the autophagy-enhancing genes listed in Table 2.

Other embodiments of the present invention relate to methods of enhancing autophagy and/or treating neurodegenerative diseases and/or proteinopathies through the inhibition of the activity of products of the autophagy-inhibiting genes listed in Table 3 or the enhancement of the activity of products of the autophagy-enhancing genes listed in Table 4. Other embodiments of the present invention relate to methods of inhibiting autophagy and/or treating cancer or pancreatitis through the enhancement of the activity of products of the autophagy-inhibiting genes listed in Table 3 or the inhibition of the activity of products of the autophagy-enhancing genes listed in Table 4.

TABLE 3
Autophagy-inhibiting genes.

| Gene | | | |
|---|---|---|---|
| Symbol | Gene ID | Genbank Acc. No. | Gene Name |
| GHSR | 2693 | NM_004122 | growth hormone secretagogue receptor |
| TINP1 | 10412 | NM_014886 | TGF beta-inducible nuclear protein 1 |
| CHAF1B | 8208 | NM_005441 | chromatin assembly factor 1, subunit B (p60) |
| COX5A | 9377 | NM_004255 | cytochrome c oxidase subunit Va |
| IHPK3 | 117283 | NM_054111 | inositol hexaphosphate kinase 3 |
| CENPE | 1062 | NM_001813 | centromere protein E, 312 kDa |
| CLCF1 | 23529 | NM_013246 | cardiotrophin-like cytokine factor 1 |
| KIAA0133 | 9816 | XM_375851 | KIAA0133 |
| ADMR | 11318 | NM_007264 | adrenomedullin receptor |
| OGDH | 4967 | NM_002541 | oxoglutarate (alpha-ketoglutarate) dehydrogenase |
| | | | (lipoamide) |
| DDX24 | 57062 | NM_020414 | DEAD (Asp-Glu-Ala-Asp, SEQ ID NO: 1) box |
| | | | polypeptide 24 |
| NUPR1 | 26471 | NM_012385 | nuclear protein 1 |
| FXYD2 | 486 | NM_001680 | FXYD domain containing ion transport regulator 2 |
| TRHR | 7201 | NM_003301 | thyrotropin-releasing hormone receptor |
| SUV39H1 | 6839 | NM_003173 | suppressor of variegation 3-9 homolog 1 |
| | | | (Drosophila) |
| FCER1A | 2205 | NM_002001 | Fc fragment of IgE, high affinity I, receptor for; alpha |
| | | | polypeptide |
| PTPRU | 10076 | NM_005704 | protein tyrosine phosphatase, receptor type, U |
| GPX2 | 2877 | NM_002083 | glutathione peroxidase 2 (gastrointestinal) |
| EP300 | 2033 | NM_001429 | E1A binding protein p300 |
| LOC388959 | 388959 | XM_373989 | hypothetical LOC388959 |
| NTN2L | 4917 | NM_006181 | netrin 2-like (chicken) |
| DOCK8 | 81704 | NM_203447 | dedicator of cytokinesis 8 |
| MAP3K7IP1 | 10454 | NM_006116 | mitogen-activated protein kinase kinase kinase 7 |
| | | | interacting protein 1 |
| PLAGL2 | 5326 | NM_002657 | pleiomorphic adenoma gene-like 2 |
| NUDT1 | 4521 | NM_002452 | nudix (nucleoside diphosphate linked moiety X)- |
| | | | type motif 1 |
| RELN | 5649 | NM_005045 | reelin |
| PNKD | 25953 | NM_015488 | paroxysmal nonkinesiogenic dyskinesia |
| GNG5 | 2787 | NM_005274 | guanine nucleotide binding protein (G protein), |
| | | | gamma 5 |
| CHKA | 1119 | NM_001277 | choline kinase alpha |
| C5AR1 | 728 | NM_001736 | complement component 5a receptor 1 |
| SCOTIN | 51246 | NM_016479 | scotin |
| PIGY | 84992 | NM_032906 | phosphatidylinositol glycan anchor biosynthesis, |
| | | | class Y |
| NAGK | 55577 | NM_017567 | N-acetylglucosamine kinase |
| RAGE | 5891 | NM_014226 | renal tumor antigen |
| USP24 | 23358 | XM_165973 | ubiquitin specific peptidase 24 |
| AURKA | 6790 | NM_003600 | aurora kinase A |
| PLDN | 26258 | NM_012388 | pallidin homolog (mouse) |
| PPARD | 5467 | NM_006238 | peroxisome proliferator-activated receptor delta |
| HRC | 3270 | NM_002152 | histidine rich calcium binding protein |
| NNMT | 4837 | NM_006169 | nicotinamide N-methyltransferase |
| COPB2 | 9276 | NM_004766 | coatomer protein complex, subunit beta 2 (beta |
| | | | prime) |
| CDK5RAP3 | 80279 | NM_025197 | CDK5 regulatory subunit associated protein 3 |
| NLK | 51701 | NM_016231 | nemo-like kinase |
| PFKL | 5211 | NM_002626 | phosphofructokinase, liver |
| RNPEPL1 | 57140 | NM_018226 | arginyl aminopeptidase (aminopeptidase B)-like 1 |
| EPHA6 | 203806 | XM_114973 | EPH receptor A6 |
| CDCA8 | 55143 | NM_018101 | cell division cycle associated 8 |
| CKAP5 | 9793 | NM_014756 | cytoskeleton associated protein 5 |
| ZBTB16 | 7704 | NM_006006 | zinc finger and BTB domain containing 16 |
| GABBR2 | 9568 | NM_005458 | gamma-aminobutyric acid (GABA) B receptor, 2 |
| PTMA | 5757 | NM_002823 | prothymosin, alpha (gene sequence 28) |
| PTCRA | 171558 | NM_138296 | pre T-cell antigen receptor alpha |
| RORC | 6097 | NM_005060 | RAR-related orphan receptor C |
| GNAI1 | 2770 | NM_002069 | guanine nucleotide binding protein (G protein), |
| | | | alpha inhibiting activity polypeptide 1 |
| UTS2R | 2837 | NM_018949 | urotensin 2 receptor |
| MATN3 | 4148 | NM_002381 | matrilin 3 |
| NPTX1 | 4884 | NM_002522 | neuronal pentraxin I |
| SP140 | 11262 | NM_007237 | SP140 nuclear body protein |
| SMARCD1 | 6602 | NM_003076 | SWI/SNF related, matrix associated, actin |
| | | | dependent regulator of chromatin, subfamily d, |
| | | | member 1 |
| PAK6 | 56924 | NM_020168 | p21(CDKN1A)-activated kinase 6 |
| TACR2 | 6865 | NM_001057 | tachykinin receptor 2 |
| MMP17 | 4326 | NM_016155 | matrix metallopeptidase 17 (membrane-inserted) |
| MUC3A | 4584 | XM_374502 | mucin 3A, cell surface associated |
| PRKCZ | 5590 | NM_002744 | protein kinase C, zeta |
| TNFRSF17 | 608 | NM_001192 | tumor necrosis factor receptor superfamily, member |
| | | | 17 |
| GTF2IRD2 | 84163 | NM_173537 | GTF2I repeat domain containing 2 |
| TRPM3 | 80036 | NM_020952 | transient receptor potential cation channel, |
| | | | subfamily M, member 3 |
| HMBS | 3145 | NM_000190, | hydroxymethylbilane synthase |
| | | NM_176954 | |
| CYP27A1 | 1593 | NM_000784 | cytochrome P450, family 27, subfamily A, |
| | | | polypeptide 1 |
| FBXL20 | 84961 | NM_032875 | F-box and leucine-rich repeat protein 20 |
| CD300C | 10871 | NM_006678 | CD300c molecule |
| PSD | 5662 | NM_002779 | pleckstrin and Sec7 domain containing |
| FRAG1 | 27315 | NM_014489 | FGF receptor activating protein 1 |
| PCGF1 | 84759 | NM_032673 | polycomb group ring finger 1 |
| SIX2 | 10736 | NM_016932 | sine oculis homeobox homolog 2 (Drosophila) |
| CLCN1 | 1180 | NM_000083 | chloride channel 1, skeletal muscle (Thomsen |
| | | | disease, autosomal dominant) |
| EVL | 51466 | NM_016337 | Enah/Vasp-like |
| TOM1 | 10043 | NM_005488 | target of myb1 (chicken) |
| BAIAP2 | 10458 | NM_006340 | BAI1-associated protein 2 |
| ZFY | 7544 | NM_003411 | zinc finger protein, Y-linked |
| UBE2D1 | 7321 | NM_003338 | ubiquitin-conjugating enzyme E2D 1 (UBC4/5 |
| | | | homolog, yeast) |
| GJA4 | 2701 | NM_002060 | gap junction protein, alpha 4, 37 kDa |
| SF3A2 | 8175 | NM_007165 | splicing factor 3a, subunit 2, 66 kDa |
| TRNT1 | 51095 | NM_016000 | tRNA nucleotidyl transferase, CCA-adding, 1 |
| RANGAP1 | 5905 | NM_002883 | Ran GTPase activating protein 1 |
| CCT4 | 10575 | NM_006430 | chaperonin containing TCP1, subunit 4 (delta) |
| TSPAN4 | 7106 | NM_003271 | tetraspanin 4 |
| PTGER2 | 5732 | NM_000956 | prostaglandin E receptor 2 (subtype EP2), 53 kDa |
| GTPBP4 | 23560 | NM_012341 | GTP binding protein 4 |
| ADRA1A | 148 | NM_000680 | adrenergic, alpha-1A-, receptor |
| PHB2 | 11331 | NM_007273 | prohibitin 2 |
| TNFRSF19L | 84957 | NM_032871 | tumor necrosis factor receptor superfamily, member |
| | | | 19-like |
| COL14A1 | 7373 | XM_044622 | collagen, type XIV, alpha 1 (undulin) |
| CD79A | 973 | NM_001783 | CD79a molecule, immunoglobulin-associated alpha |
| F12 | 2161 | NM_000505 | coagulation factor XII (Hageman factor) |
| ASMT | 438 | NM_004043 | acetylserotonin O-methyltransferase |
| GRK6 | 2870 | NM_002082 | G protein-coupled receptor kinase 6 |
| GNRH2 | 2797 | NM_001501 | gonadotropin-releasing hormone 2 |
| SDHB | 6390 | NM_003000 | succinate dehydrogenase complex, subunit B, iron |
| | | | sulfur (Ip) |
| THBS2 | 7058 | NM_003247 | thrombospondin 2 |
| HIVEP2 | 3097 | NM_145975, | human immunodeficiency virus type I enhancer |
| | | NM_006734 | binding protein 2 |
| WASF1 | 8936 | NM_003931 | WAS protein family, member 1 |
| SSPN | 8082 | NM_005086 | sarcospan (Kras oncogene-associated gene) |
| ITGAV | 3685 | NM_002210 | integrin, alpha V (vitronectin receptor, alpha |
| | | | polypeptide, antigen CD51) |
| PLXNA2 | 5362 | XM_372810 | plexin A2 |
| NCR3 | 259197 | NM_147130 | natural cytotoxicity triggering receptor 3 |
| TH | 7054 | NM_000360 | tyrosine hydroxylase |
| HMGCL | 3155 | NM_177229, | 3-hydroxymethyl-3-methylglutaryl-Coenzyme A |
| | | NM_000191 | lyase (hydroxymethylglutaricaciduria) |
| CENPJ | 55835 | NM_018451 | centromere protein J |
| FABP1 | 2168 | NM_001443 | fatty acid binding protein 1, liver |
| CASP1 | 834 | NM_001223 | caspase 1, apoptosis-related cysteine peptidase |
| | | | (interleukin 1, beta, convertase) |
| MCCC1 | 56922 | NM_020166 | methylcrotonoyl-Coenzyme A carboxylase 1 (alpha) |
| DBX1 | 120237 | XM_061930 | developing brain homeobox 1 |
| KIAA0196 | 9897 | NM_014846 | KIAA0196 |
| HLA-DRB1 | 3123 | NM_002124, | major histocompatibility complex, class II, DR beta 1 |
| | | NM_172672 | |
| MMACHC | 25974 | XM_032397 | methylmalonic aciduria (cobalamin deficiency) cblC |
| | | | type, with homocystinuria |
| TGFBI | 7045 | NM_000358 | transforming growth factor, beta-induced, 68 kDa |
| PPFIA4 | 8497 | XM_046751 | protein tyrosine phosphatase, receptor type, f |
| | | | polypeptide (PTPRF), interacting protein (liprin), |
| | | | alpha 4 |
| SORCS2 | 57537 | NM_020777 | sortilin-related VPS10 domain containing receptor 2 |
| BAI3 | 577 | NM_001704 | brain-specific angiogenesis inhibitor 3 |
| RFX1 | 5989 | NM_002918 | regulatory factor X, 1 (influences HLA class II |
| | | | expression) |
| IRAK3 | 11213 | NM_007199 | interleukin-1 receptor-associated kinase 3 |
| PA2G4 | 5036 | NM_006191 | proliferation-associated 2G4, 38 kDa |
| GCM2 | 9247 | NM_004752 | glial cells missing homolog 2 (Drosophila) |
| CHRND | 1144 | NM_000751 | cholinergic receptor, nicotinic, delta |
| USP54 | 159195 | NM_152586 | ubiquitin specific peptidase 54 |
| HNRPU | 3192 | NM_004501 | heterogeneous nuclear ribonucleoprotein U |
| | | | (scaffold attachment factor A) |
| NUTF2 | 10204 | NM_005796 | nuclear transport factor 2 |
| HNRPK | 3190 | NM_002140 | heterogeneous nuclear ribonucleoprotein K |
| ARCN1 | 372 | NM_001655 | archain 1 |
| TRAF1 | 7185 | NM_005658 | TNF receptor-associated factor 1 |
| TUBB2A | 7280 | NM_001069 | tubulin, beta 2A |
| ATG16L2 | 89849 | XM_058426 | ATG16 autophagy related 16-like 2 (S. cerevisiae) |
| ARSE | 415 | NM_000047 | arylsulfatase E (chondrodysplasia punctata 1) |
| SIDT1 | 54847 | NM_017699 | SID1 transmembrane family, member 1 |
| GNG11 | 2791 | NM_004126 | guanine nucleotide binding protein (G protein), |
| | | | gamma 11 |
| NAT9 | 26151 | NM_015654 | N-acetyltransferase 9 |
| MMP10 | 4319 | NM_002425 | matrix metallopeptidase 10 (stromelysin 2) |
| HOXD11 | 3237 | NM_021192 | homeobox D11 |
| POLR3G | 10622 | NM_006467 | polymerase (RNA) III (DNA directed) polypeptide G |
| | | | (32 kD) |
| TACC2 | 10579 | NM_006997 | transforming, acidic coiled-coil containing protein 2 |
| BGN | 633 | NM_001711 | biglycan |
| C11orf68 | 83638 | NM_031450 | chromosome 11 open reading frame 68 |
| QSCN6 | 5768 | NM_002826 | quiescin Q6 |
| TRIM8 | 81603 | NM_030912 | tripartite motif-containing 8 |
| GJA3 | 2700 | NM_021954, | gap junction protein, alpha 3, 46 kDa |
| | | NM_029726 | |
| TMPRSS5 | 80975 | NM_030770 | transmembrane protease, serine 5 (spinesin) |
| TAF2 | 6873 | NM_003184 | TAF2 RNA polymerase II, TATA box binding protein |
| | | | (TBP)-associated factor, 150 kDa |
| OA48-18 | 10414 | NM_006107 | acid-inducible phosphoprotein |
| MKLN1 | 4289 | NM_013255 | muskelin 1, intracellular mediator containing kelch |
| | | | motifs |
| USP19 | 10869 | XM_496642 | ubiquitin specific peptidase 19 |
| SETDB1 | 9869 | NM_012432 | SET domain, bifurcated 1 |
| SLC25A19 | 60386 | NM_021734 | solute carrier family 25 (mitochondrial thiamine |
| | | | pyrophosphate carrier), member 19 |
| PTPRH | 5794 | NM_002842 | protein tyrosine phosphatase, receptor type, H |
| INTS4 | 92105 | NM_033547 | integrator complex subunit 4 |
| COPE | 11316 | NM_007263 | coatomer protein complex, subunit epsilon |
| PRKAG3 | 53632 | NM_017431 | protein kinase, AMP-activated, gamma 3 non- |
| | | | catalytic subunit |
| BPGM | 669 | NM_001724 | 2,3-bisphosphoglycerate mutase |
| PRAF2 | 11230 | NM_007213 | PRA1 domain family, member 2 |
| NFIL3 | 4783 | NM_005384 | nuclear factor, interleukin 3 regulated |
| CXCL12 | 6387 | NM_000609 | chemokine (C-X-C motif) ligand 12 (stromal cell- |
| | | | derived factor 1) |
| PLCH2 | 9651 | XM_371214 | phospholipase C, eta 2 |
| CHID1 | 66005 | NM_023947 | chitinase domain containing 1 |
| CEND1 | 51286 | NM_016564 | cell cycle exit and neuronal differentiation 1 |
| HIST2H3C | 126961 | NM_021059 | histone cluster 2, H3c |
| CNKSR2 | 22866 | NM_014927 | connector enhancer of kinase suppressor of Ras 2 |
| MYL3 | 4634 | NM_000258 | myosin, light chain 3, alkali; ventricular, skeletal, |
| | | | slow |
| SORBS3 | 10174 | NM_005775 | sorbin and SH3 domain containing 3 |
| PFDN2 | 5202 | NM_012394 | prefoldin subunit 2 |
| RBBP8 | 5932 | NM_002894 | retinoblastoma binding protein 8 |
| PROSC | 11212 | NM_007198 | proline synthetase co-transcribed homolog |
| | | | (bacterial) |
| TRIP6 | 7205 | NM_003302 | thyroid hormone receptor interactor 6 |
| HSFY2 | 159119 | NM_153716 | heat shock transcription factor, Y linked 2 |
| SCAMP4 | 113178 | NM_079834 | secretory carrier membrane protein 4 |
| TRPA1 | 8989 | NM_007332 | transient receptor potential cation channel, |
| | | | subfamily A, member 1 |
| HNRPM | 4670 | NM_005968 | heterogeneous nuclear ribonucleoprotein M |
| C2orf13 | 200558 | NM_173545 | chromosome 2 open reading frame 13 |
| AGER | 177 | NM_001136 | advanced glycosylation end product-specific |
| | | | receptor |
| GFER | 2671 | NM_005262 | growth factor, augmenter of liver regeneration |
| | | | (ERV1 homolog, S. cerevisiae) |
| ERH | 2079 | NM_004450 | enhancer of rudimentary homolog (Drosophila) |
| PAQR6 | 79957 | NM_024897 | progestin and adipoQ receptor family member VI |
| UNC13B | 10497 | NM_006377 | unc-13 homolog B (C. elegans) |
| EGLN2 | 112398 | NM_053046 | egl nine homolog 2 (C. elegans) |
| FGFR1 | 2260 | NM_000604 | fibroblast growth factor receptor 1 (fms-related |
| | | | tyrosine kinase 2, Pfeiffer syndrome) |
| CARKL | 23729 | NM_013276 | carbohydrate kinase-like |
| SEMA4B | 10509 | NM_020210 | sema domain, immunoglobulin domain (Ig), |
| | | | transmembrane domain (TM) and short cytoplasmic |
| | | | domain, (semaphorin) 4B |
| TUBGCP6 | 85378 | NM_020461 | tubulin, gamma complex associated protein 6 |
| ICT1 | 3396 | NM_001545, | immature colon carcinoma transcript 1 |
| | | NM_016879 | |
| WFDC2 | 10406 | NM_006103 | WAP four-disulfide core domain 2 |
| CPNE6 | 9362 | NM_006032 | copine VI (neuronal) |
| CAMKV | 79012 | NM_024046 | CaM kinase-like vesicle-associated |
| LOC285643 | 285643 | XM_209695 | KIF4B |
| C18orf8 | 29919 | NM_013326 | chromosome 18 open reading frame 8 |
| LOR | 4014 | NM_000427 | loricrin |
| ADM | 133 | NM_001124 | adrenomedullin |
| KIF11 | 3832 | NM_004523 | kinesin family member 11 |
| FANCC | 2176 | NM_000136 | Fanconi anemia, complementation group C |
| NOXO1 | 124056 | NM_144603 | NADPH oxidase organizer 1 |
| UBE1L2 | 55236 | NM_018227 | ubiquitin-activating enzyme E1-like 2 |
| P2RX1 | 5023 | NM_002558 | purinergic receptor P2X, ligand-gated ion channel, 1 |
| NPTN | 27020 | NM_012428 | neuroplastin |
| PDCD5 | 9141 | NM_004708 | programmed cell death 5 |

TABLE 4
Autophagy-enhancing genes.

| Gene | | Genbank Acc. | |
|---|---|---|---|
| Symbol | Gene ID | No. | Gene Name |
| SMYD3 | 64754 | NM_022743 | SET and MYND domain |
| | | | containing 3 |
| TCEB3 | 6924 | NM_003198 | transcription elongation |
| | | | factor B (SIII), polypeptide 3 |
| | | | (110 kDa, elongin A) |
| CATSPER4 | 378807 | XM_371237 | cation channel, sperm |
| | | | associated 4 |
| MEGF10 | 84466 | NM_032446 | multiple EGF-like-domains 10 |
| KIF5C | 3800 | XM_377774 | kinesin family member 5C |
| RELA | 5970 | NM_021975 | v-rel reticuloendotheliosis |
| | | | viral oncogene homolog A, |
| | | | nuclear factor of kappa light |
| | | | polypeptide gene enhancer in |
| | | | B-cells 3, p65 (avian) |
| GAB1 | 2549 | NM_002039 | GRB2-associated binding |
| | | | protein 1 |
| LOC285647 | 285647 | XM_209700 | suppressor of defective |
| | | | silencing 3 pseudogene |
| GPR18 | 2841 | NM_005292, | G protein-coupled receptor 18 |
| | | NM_145948 | |
| PDCL | 5082 | NM_005388 | phosducin-like |
| STIM1 | 6786 | NM_003156 | stromal interaction molecule 1 |
| NFKB1 | 4790 | NM_003998 | Nuclear factor of kappa light |
| | | | polypeptide gene enhancer |
| | | | in B-cells 1 |
| TPR | 7175 | NM_003292 | translocated promoter region |
| | | | (to activated MET oncogene) |
| PGGT1B | 5229 | NM_005023 | protein |
| | | | geranylgeranyltransferase |
| | | | type I, beta subunit |

The products of the autophagy-related genes of the invention can be classified into a number of non-mutually exclusive categories. For example, certain gene products of the present invention can be classified as oxidoreductases, receptors, proteases, ligases, kinases, synthases, synthetases, chaperones, hydrolases, membrane traffic proteins, calcium binding proteins and/or regulatory molecules. The classification of selected autophagy-inhibiting gene products is listed in Table 5, while the classification of selected autophagy-enhancing gene products is listed in Table 6. Since certain types of agents are better suited for the modulation of the activity of a specific class of gene product, in some embodiments the present invention is directed towards the modulation of one or more class of autophagy-related gene product.

TABLE 5
Classification of certain autophagy-inhibiting gene products.

| Gene | | |
|---|---|---|
| Symbol | Gene Name | Class |
| CYP27A1 | cytochrome P450, family 27, | Oxidoreductase |
| | subfamily A, polypeptide 1; | |
| | CYP27A1 | |
| SDHB | succinate dehydrogenase complex, | Oxidoreductase |
| | subunit B, iron sulfur (Ip); SDHB | |
| OGDH | oxoglutarate (alpha-ketoglutarate) | Oxidoreductase |
| | dehydrogenase (lipoamide); OGDH | |
| QSCN6 | quiescin Q6; QSCN6 | Oxidoreductase |
| EGLN2 | egl nine homolog 2 (C. elegans); | Oxidoreductase |
| | EGLN2 | |
| TH | tyrosine hydroxylase; TH | Oxidoreductase |
| COX5A | cytochrome c oxidase subunit Va; | Oxidoreductase |
| | COX5A | |
| SOD1 | superoxide dismutase 1, soluble | Oxidoreductase |
| | (amyotrophic lateral sclerosis 1 | |
| | (adult); SOD1 | |
| GPX2 | glutathione peroxidase 2 | Oxidoreductase |
| | (gastrointestinal); GPX2 | |
| GFER | growth factor, augmenter of liver | Oxidoreductase |
| | regeneration (ERV1 homolog, | |
| | S. cerevisiae); GFER | |
| TRPM3 | transient receptor potential cation, | Receptor |
| | channel subfamily M, member 3; | |
| | TRPM3 | |
| TMPRSS5 | transmembrane protease, serine 5 | Receptor |
| | (spinesin); TMPRSS5 | |
| IRAK3 | interleukin-1 receptor-associated | Receptor |
| | kinase 3; IRAK3 | |
| ADMR(Also | adrenomedullin receptor; ADMR | Receptor |
| Known as | | |
| GPR182) | | |
| FGFR1 | fibroblast growth factor receptor 1 | Receptor |
| | (fms-related tyrosine kinase 2, | |
| | Pfeiffer syndrome); FGFR1 | |
| UNC13B | unc-13 homolog B (C. elegans); | Receptor |
| | UNC13B | |
| PTGER2 | prostaglandin E receptor 2 (subtype | Receptor |
| | EP2), 53 kDa; PTGER2 | |
| AGER | advanced glycosylation end product- | Receptor |
| | specific receptor; AGER | |
| BGN | biglycan; BGN | Receptor |
| GABBR2 | gamma-aminobutyric acid (GABA) B | Receptor |
| | receptor, 2; GABBR2 | |
| PPARD | peroxisome proliferator-activated | Receptor |
| | receptor delta; PPARD | |
| GHSR | growth hormone secretagogue | Receptor |
| | receptor; GHSR | |
| BAIAP2 | BAI1-associated protein 2; BAIAP2 | Receptor |
| SORCS2 | sortilin-related VPS10 domain | Receptor |
| | containing receptor 2; SORCS2 | |
| PAQR6 | progestin and adipoQ receptor family | Receptor |
| | member VI; PAQR6 | |
| EPHA6 | EPH receptor A6; EPHA6 | Receptor |
| TRHR | thyrotropin-releasing hormone | Receptor |
| | receptor; TRHR | |
| C5AR1 | complement component 5a receptor | Receptor |
| | 1; C5AR1 | |
| BAI3 | brain-specific angiogenesis inhibitor | Receptor |
| | 3; BAI3 | |
| TLR3 | toll-like receptor 3; TLR3 | Receptor |
| PTPRH | protein tyrosine phosphatase, receptor | Receptor |
| | type, H; PTPRH | |
| ADRA1A | adrenergic, alpha-1A-, receptor; | Receptor |
| | ADRA1A | |
| UTS2R | urotensin 2 receptor; UTS2R | Receptor |
| RORC | RAR-related orphan receptor C; | Receptor |
| | RORC | |
| CHRND | cholinergic receptor, nicotinic, | Receptor |
| | delta; CHRND | |
| TACR2 | tachykinin receptor 2; TACR2 | Receptor |
| P2RX1 | purinergic receptor P2X, ligand-gated | Receptor |
| | ion channel, 1; P2RX1 | |
| PLXNA2 | plexin A2; PLXNA2 | Receptor |
| PTPRU | protein tyrosine phosphatase, receptor | Receptor |
| | type, U; PTPRU | |
| FCER1A | Fc fragment of IgE, high affinity I, | Receptor |
| | receptor for; alpha polypeptide; | |
| | FCER1A | |
| CD300C | CD300c molecule; CD300C | Receptor |
| TNFRSF19L | tumor necrosis factor receptor | Receptor |
| (Also known | superfamily, member 19-like; | |
| as RELT) | TNFRSF19L | |
| TMPRSS5 | transmembrane protease, serine 5 | Protease |
| | (spinesin); TMPRSS5 | |
| USP19 | ubiquitin specific peptidase 19; | Protease |
| | USP19 | |
| RNPEPL1 | arginyl aminopeptidase | Protease |
| | (aminopeptidase B)-like 1; | |
| | RNPEPL1 | |
| MMP10 | matrix metallopeptidase 10 | Protease |
| | (stromelysin 2); MMP10 | |
| RELN | reelin; RELN | Protease |
| F12 | coagulation factor XII (Hageman | Protease |
| | factor); F12 | |
| CASP1 | caspase 1, apoptosis-related cysteine | Protease |
| | peptidase (interleukin 1, beta, | |
| | convertase); CASP1 | |
| MMP17 | matrix metallopeptidase 17 | Protease |
| | (membrane-inserted); MMP17 | |
| CAPN1 | calpain 1, (mu/l) large subunit; | Protease |
| | CAPN1 | |
| TRIM8 | tripartite motif-containing 8; TRIM8 | Ligase |
| UBE1L2(Also | ubiquitin-activating enzyme E1-like | Ligase |
| known as | 2; UBE1L2 | |
| UBA6) | | |
| MCCC1 | methylcrotonoyl-Coenzyme A | Ligase |
| | carboxylase 1 (alpha); MCCC1 | |
| TRIM69 | tripartite motif-containing 69; | Ligase |
| | TRIM69 | |
| UBE2D1 | ubiquitin-conjugating enzyme E2D 1 | Ligase |
| | (UBC4/5 homolog, yeast); UBE2D1 | |
| HMGCL | 3-hydroxymethyl-3-methylglutaryl- | Lyase |
| | Coenzyme A lyase | |
| | (hydroxymethylglutaricaciduria); | |
| | HMGCL | |
| PAK6 | p21(CDKN1A)-activated kinase 6; | Kinase |
| | PAK6 | |
| CHKA | choline kinase alpha; CHKA | Kinase |
| RAGE | renal tumor antigen; RAGE | Kinase |
| IHPK3(Also | inositol hexaphosphate kinase 3; | Kinase |
| known as | IHPK3 | |
| IP6K3) | | |
| CAMKV | CaM kinase-like vesicle- | Kinase |
| | associated; CAMKV | |
| PRKAA2 | protein kinase, AMP-activated, alpha | Kinase |
| | 2 catalytic subunit; PRKAA2 | |
| PRKCZ | protein kinase C, zeta; PRKCZ | Kinase |
| PRKCA | protein kinase C, alpha; PRKCA | Kinase |
| CARKL(Also | carbohydrate kinase-like; CARKL | Kinase |
| known as | | |
| SHPK) | | |
| PFKL | phosphofructokinase, liver; PFKL | Kinase |
| NLK | nemo-like kinase; NLK | Kinase |
| AURKA | aurora kinase A; AURKA | Kinase |
| PROSC | proline synthetase co-transcribed | Synthase & |
| | homolog (bacterial); PROSC | synthetase |
| CCT4 | chaperonin containing TCP1, subunit | Chaperone |
| | 4 (delta); CCT4 | |
| PFDN2 | prefoldin subunit 2; PFDN2 | Chaperone |
| CHID1 | chitinase domain containing 1; | Hydrolase |
| | CHID1 | |
| ARSE | arylsulfatase E (chondrodysplasia | Hydrolase |
| | punctata 1); ARSE | |
| PLCH2 | phospholipase C, eta 2; PLCH2 | Hydrolase |
| HMBS | hydroxymethylbilane synthase; | Hydrolase |
| | HMBS | |
| PNKD | paroxysmal nonkinesiogenic | Hydrolase |
| | dyskinesia; PNKD | |
| NUDT1 | nudix (nucleoside diphosphate linked | Hydrolase |
| | moiety X)-type motif 1; NUDT1 | |
| COPB2 | coatomer protein complex, subunit | Membrane traffic |
| | beta 2 (beta prime); COPB2 | protein |
| ARCN1 | archain 1; ARCN1 | Membrane traffic |
| | | protein |
| CPNE6 | copine VI (neuronal); CPNE6 | Membrane traffic |
| | | protein |
| COPE | coatomer protein complex, subunit | Membrane traffic |
| | epsilon; COPE | protein |
| HRC | histidine rich calcium binding | Calcium binding |
| | protein; HRC | protein |
| MYL3 | myosin, light chain 3, alkali; | Calcium binding |
| | ventricular, skeletal, slow; MYL3 | protein |
| RANGAP1 | Ran GTPase activating protein | Regulatory |
| | 1; RANGAP1 | molecule |
| GTPBP4 | GTP binding protein 4; GTPBP4 | Regulatory |
| | | molecule |
| TRIP6 | thyroid hormone receptor interactor | Regulatory |
| | 6; TRIP6 | molecule |
| CNKSR2 | connector enhancer of kinase | Regulatory |
| | suppressor of Ras 2; CNKSR2 | molecule |
| PSD | pleckstrin and Sec7 domain | Regulatory |
| | containing; PSD | molecule |
| DOCK8 | dedicator of cytokinesis 8; DOCK8 | Regulatory |
| | | molecule |
| THBS2 | thrombospondin 2; THBS2 | Regulatory |
| | | molecule |
| GNAI1 | guanine nucleotide binding protein (G | Regulatory |
| | protein), alpha inhibiting activity | molecule |
| | polypeptide 1; GNAI1 | |
| FRAG1 | FGF receptor activating protein | Regulatory |
| | 1; unassigned | molecule |
| RAB7A | RAB7, member RAS oncogene | Regulatory |
| | family; RAB7 | molecule |
| CDKN2D | cyclin-dependent kinase inhibitor | Regulatory |
| | 2D (p19, inhibits CDK4); | molecule |
| | CDKN2D | |
| GNG5 | guanine nucleotide binding protein (G | Regulatory |
| | protein), gamma 5; GNG5 | molecule |
| GNG11 | guanine nucleotide binding protein (G | Regulatory |
| | protein), gamma 11; GNG11 | molecule |
| PDCD5 | programmed cell death 5; PDCD5 | Regulatory |
| | | molecule |
| WFDC2 | WAP four-disulfide core domain 2; | Regulatory |
| | WFDC2 | molecule |

TABLE 6
Classification of certain autophagy-enhancing gene products.

| Gene | | |
|---|---|---|
| Symbol | Gene Name | Class |
| TPR | translocated promoter region (to activated | Receptor |
| | MET oncogene); TPR | |
| GPR18 | G protein-coupled receptor 18; GPR18 | Receptor |
| PDCL | phosducin-like; PDCL | Regulatory |
| | | molecule |

### 3. MODULATORS OF AUTOPHAGY-RELATED GENE PRODUCTS

Certain embodiments of the present invention relate to methods of modulating autophagy or treating autophagy-related diseases (e.g. neurodegenerative disease, liver disease, muscle disease, cancer, pancreatitis). These methods involve administering an agent that modulates the activity of one or more autophagy-related gene products of the invention. In certain embodiments, methods of the invention include treatment of autophagy-related diseases by administering to a subject an agent which decreases the activity of one or more products of the genes listed in Tables 1-4. In other embodiments, methods of the invention include treatment of autophagy-related diseases by administering to a subject an agent which increases the activity of one or more products of the genes listed in Tables 1-4. Agents which may be used to modulate the activity of a gene product listed in Tables 1-4, and to thereby treat or prevent an autophagy-related disease, include antibodies (e.g., conjugated antibodies), proteins, peptides, small molecules, RNA interfering agents, e.g., siRNA molecules, ribozymes, and antisense oligonucleotides.

Any agent that modulates the activity of an autophagy-related gene product of the invention can be used to practice certain methods of the invention. Such agents can be those described herein, those known in the art, or those identified through routine screening assays (e.g. the screening assays described herein).

In some embodiments, assays used to identify agents useful in the methods of the present invention include a reaction between the autophagy-related gene product and one or more assay components. The other components may be either a test compound (e.g. the potential agent), or a combination of test compounds and a natural binding partner of the autophagy-related gene product. Agents identified via such assays, such as those described herein, may be useful, for example, for modulating autophagy and treating autophagy-related diseases.

Agents useful in the methods of the present invention may be obtained from any available source, including systematic libraries of natural and/or synthetic compounds. Agents may also be obtained by any of the numerous approaches in combinatorial library methods known in the art, including: biological libraries; peptoid libraries (libraries of molecules having the functionalities of peptides, but with a novel, non-peptide backbone which are resistant to enzymatic degradation but which nevertheless remain bioactive; see, e.g., Zuckermann et al., 1994, J. Med. Chem. 37:2678-85); spatially addressable parallel solid phase or solution phase libraries; synthetic library methods requiring deconvolution; the ‘one-bead one-compound’ library method; and synthetic library methods using affinity chromatography selection. The biological library and peptoid library approaches are limited to peptide libraries, while the other four approaches are applicable to peptide, non-peptide oligomer or small molecule libraries of compounds (Lam, 1997, Anticancer Drug Des. 12:145).

Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233.

Libraries of agents may be presented in solution (e.g., Houghten, 1992, Biotechniques 13:412-421), or on beads (Lam, 1991, Nature 354:82-84), chips (Fodor, 1993, Nature 364:555-556), bacteria and/or spores, (Ladner, U.S. Pat. No. 5,223,409), plasmids (Cull et al, 1992, Proc Natl Acad Sci USA 89:1865-1869) or on phage (Scott and Smith, 1990, Science 249:386-390; Devlin, 1990, Science 249:404-406; Cwirla et al, 1990, Proc. Natl. Acad. Sci. 87:6378-6382; Felici, 1991, J. Mol. Biol. 222:301-310; Ladner, supra.).

Agents useful in the methods of the present invention may be identified, for example, using assays for screening candidate or test compounds which are substrates of an autophagy-related gene product of the invention or biologically active portion thereof. In another embodiment, agents useful in the methods of the invention may be identified using assays for screening candidate or test compounds which bind to an autophagy-related gene product of the invention or a biologically active portion thereof. Determining the ability of the test compound to directly bind to an autophagy-related gene product can be accomplished, for example, by coupling the compound with a radioisotope or enzymatic label such that binding of the compound to the autophagy-related gene product can be determined by detecting the labeled compound in a complex. For example, compounds can be labeled with ¹²⁵I, ³⁵S, ¹⁴C, or ³H, either directly or indirectly, and the radioisotope detected by direct counting of radioemission or by scintillation counting. Alternatively, assay components can be enzymatically labeled with, for example, horseradish peroxidase, alkaline phosphatase, or luciferase, and the enzymatic label detected by determination of conversion of an appropriate substrate to product.

Agents useful in the methods of the invention may also be identified, for example, using assays that identify compounds which modulate (e.g., affect either positively or negatively) interactions between an autophagy-related gene product and its substrates and/or binding partners. Such compounds can include, but are not limited to, molecules such as antibodies, peptides, hormones, oligonucleotides, nucleic acids, and analogs thereof. Such compounds may also be obtained from any available source, including systematic libraries of natural and/or synthetic compounds.

The basic principle of the assay systems used to identify compounds that modulate the interaction between the autophagy-related gene product and its binding partner involves preparing a reaction mixture containing the autophagy-related gene product and its binding partner under conditions and for a time sufficient to allow the two products to interact and bind, thus forming a complex. In order to test an agent for inhibitory activity, the reaction mixture is prepared in the presence and absence of the test compound. The test compound can be initially included in the reaction mixture, or can be added at a time subsequent to the addition of the autophagy-related gene product and its binding partner. Control reaction mixtures are incubated without the test compound or with a placebo. The formation of any complexes between the autophagy-related gene product and its binding partner is then detected. The formation of a complex in the control reaction, but less or no such formation in the reaction mixture containing the test compound, indicates that the compound interferes with the interaction of the autophagy-related gene product and its binding partner. Conversely, the formation of more complex in the presence of the compound than in the control reaction indicates that the compound may enhance interaction of the autophagy-related gene product and its binding partner.

The assay for compounds that modulate the interaction of the autophagy-related gene product with its binding partner may be conducted in a heterogeneous or homogeneous format. Heterogeneous assays involve anchoring either the autophagy-related gene product or its binding partner onto a solid phase and detecting complexes anchored to the solid phase at the end of the reaction. In homogeneous assays, the entire reaction is carried out in a liquid phase. In either approach, the order of addition of reactants can be varied to obtain different information about the compounds being tested. For example, test compounds that interfere with the interaction between the autophagy-related gene products and the binding partners (e.g., by competition) can be identified by conducting the reaction in the presence of the test substance, i.e., by adding the test substance to the reaction mixture prior to or simultaneously with the autophagy-related gene product and its interactive binding partner. Alternatively, test compounds that disrupt preformed complexes, e.g., compounds with higher binding constants that displace one of the components from the complex, can be tested by adding the test compound to the reaction mixture after complexes have been formed. The various formats are briefly described below.

In a heterogeneous assay system, either the autophagy-related gene product or its binding partner is anchored onto a solid surface or matrix, while the other corresponding non-anchored component may be labeled, either directly or indirectly. In practice, microtitre plates are often utilized for this approach. The anchored species can be immobilized by a number of methods, either non-covalent or covalent, that are typically well known to one who practices the art. Non-covalent attachment can often be accomplished simply by coating the solid surface with a solution of the autophagy-related gene product or its binding partner and drying. Alternatively, an immobilized antibody specific for the assay component to be anchored can be used for this purpose.

In related assays, a fusion protein can be provided which adds a domain that allows one or both of the assay components to be anchored to a matrix. For example, glutathione-S-transferase/marker fusion proteins or glutathione-S-transferase/binding partner can be adsorbed onto glutathione sepharose beads (Sigma Chemical, St. Louis, Mo.) or glutathione derivatized microtiter plates, which are then combined with the test compound or the test compound and either the non-adsorbed autophagy-related gene product or its binding partner, and the mixture incubated under conditions conducive to complex formation (e.g., physiological conditions). Following incubation, the beads or microtiter plate wells are washed to remove any unbound assay components, the immobilized complex assessed either directly or indirectly, for example, as described above. Alternatively, the complexes can be dissociated from the matrix, and the level of autophagy-related gene product binding or activity determined using standard techniques.

A homogeneous assay may also be used to identify modulators of autophagy-related gene products. This is typically a reaction, analogous to those mentioned above, which is conducted in a liquid phase in the presence or absence of the test compound. The formed complexes are then separated from unreacted components, and the amount of complex formed is determined As mentioned for heterogeneous assay systems, the order of addition of reactants to the liquid phase can yield information about which test compounds modulate (inhibit or enhance) complex formation and which disrupt preformed complexes.

In such a homogeneous assay, the reaction products may be separated from unreacted assay components by any of a number of standard techniques, including but not limited to: differential centrifugation, chromatography, electrophoresis and immunoprecipitation. In differential centrifugation, complexes of molecules may be separated from uncomplexed molecules through a series of centrifugal steps, due to the different sedimentation equilibria of complexes based on their different sizes and densities (see, for example, Rivas, G., and Minton, A. P., Trends Biochem Sci 1993 August; 18(8):284-7). Standard chromatographic techniques may also be utilized to separate complexed molecules from uncomplexed ones. For example, gel filtration chromatography separates molecules based on size, and through the utilization of an appropriate gel filtration resin in a column format, for example, the relatively larger complex may be separated from the relatively smaller uncomplexed components. Similarly, the relatively different charge properties of the complex as compared to the uncomplexed molecules may be exploited to differentially separate the complex from the remaining individual reactants, for example through the use of ion-exchange chromatography resins. Such resins and chromatographic techniques are well known to one skilled in the art (see, e.g., Heegaard, 1998, J Mol. Recognit. 11:141-148; Hage and Tweed, 1997, J. Chromatogr. B. Biomed. Sci. Appl., 699:499-525). Gel electrophoresis may also be employed to separate complexed molecules from unbound species (see, e.g., Ausubel et al (eds.), In: Current Protocols in Molecular Biology, J. Wiley & Sons, New York. 1999). In this technique, protein or nucleic acid complexes are separated based on size or charge, for example. In order to maintain the binding interaction during the electrophoretic process, nondenaturing gels in the absence of reducing agent are typically preferred, but conditions appropriate to the particular interactants will be well known to one skilled in the art. Immunoprecipitation is another common technique utilized for the isolation of a protein-protein complex from solution (see, e.g., Ausubel et al (eds.), In: Current Protocols in Molecular Biology, J. Wiley & Sons, New York. 1999). In this technique, all proteins binding to an antibody specific to one of the binding molecules are precipitated from solution by conjugating the antibody to a polymer bead that may be readily collected by centrifugation. The bound assay components are released from the beads (through a specific proteolysis event or other technique well known in the art which will not disturb the protein-protein interaction in the complex), and a second immunoprecipitation step is performed, this time utilizing antibodies specific for the correspondingly different interacting assay component. In this manner, only formed complexes should remain attached to the beads. Variations in complex formation in both the presence and the absence of a test compound can be compared, thus offering information about the ability of the compound to modulate interactions between the autophagy-related gene product and its binding partner.

Modulators of autophagy-related gene product expression may also be identified, for example, using methods wherein a cell is contacted with a candidate compound and the expression of mRNA or protein, corresponding to an autophagy-related gene in the cell, is determined. The level of expression of mRNA or protein in the presence of the candidate compound is compared to the level of expression of mRNA or protein in the absence of the candidate compound. The candidate compound can then be identified as a modulator of autophagy-related gene product expression based on this comparison. For example, when expression of autophagy-related gene product is greater in the presence of the candidate compound than in its absence, the candidate compound is identified as a stimulator of marker mRNA or protein expression. Conversely, when expression of autophagy-related gene product is less in the presence of the candidate compound than in its absence, the candidate compound is identified as an inhibitor of marker mRNA or protein expression. The level of autophagy-related gene product expression in the cells can be determined by methods described herein for detecting marker mRNA or protein.

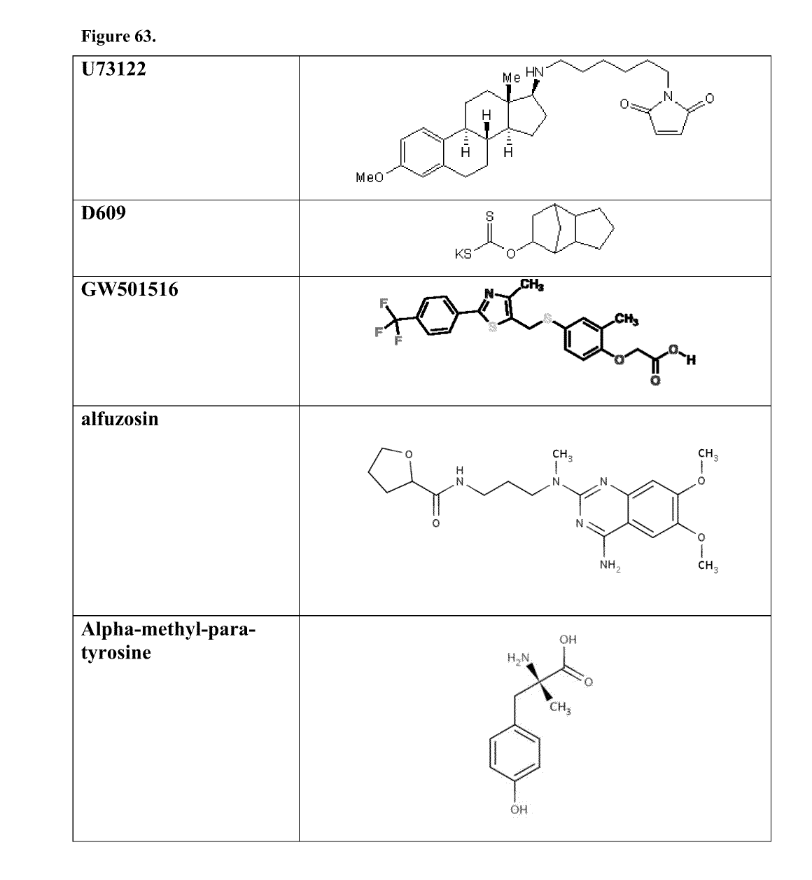

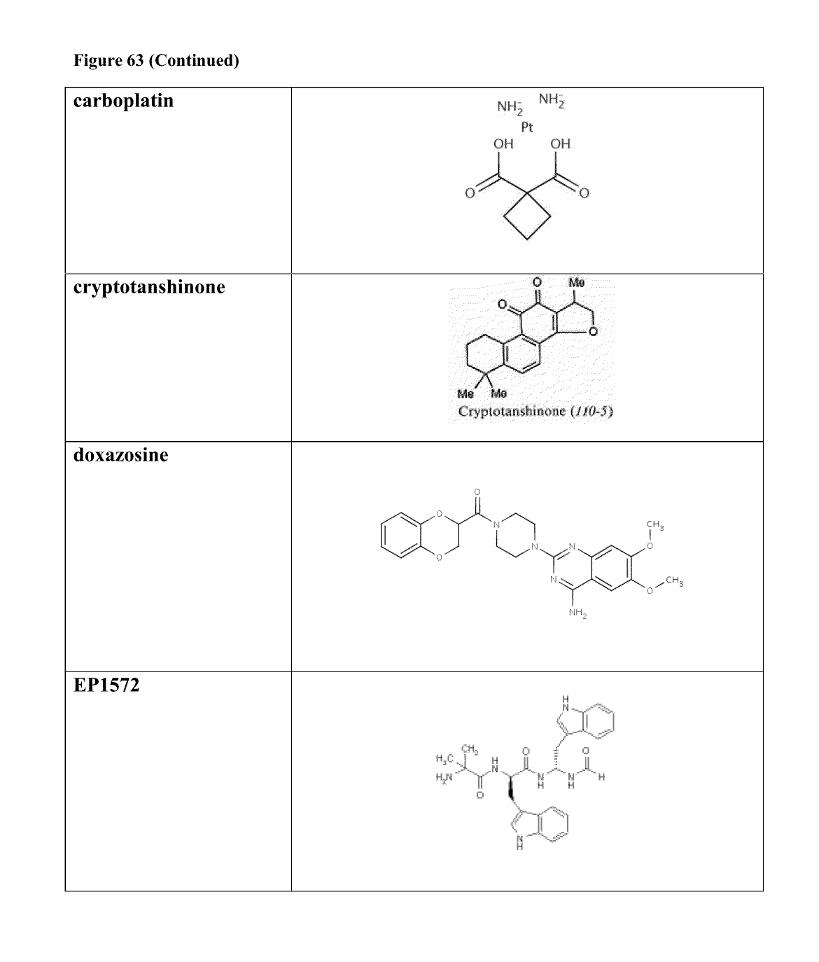

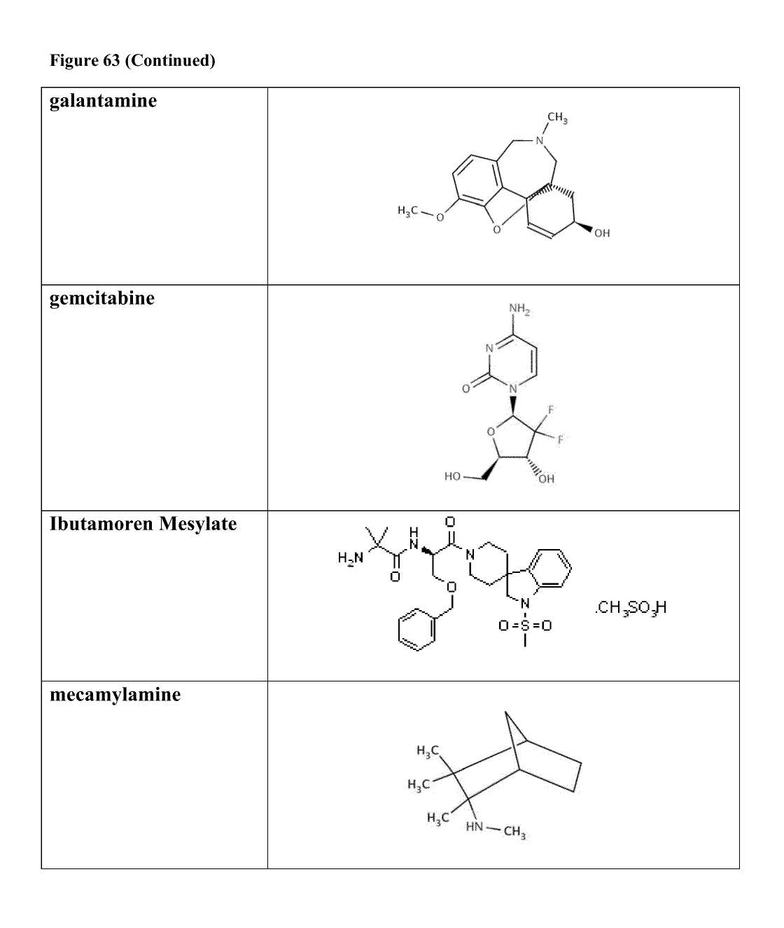

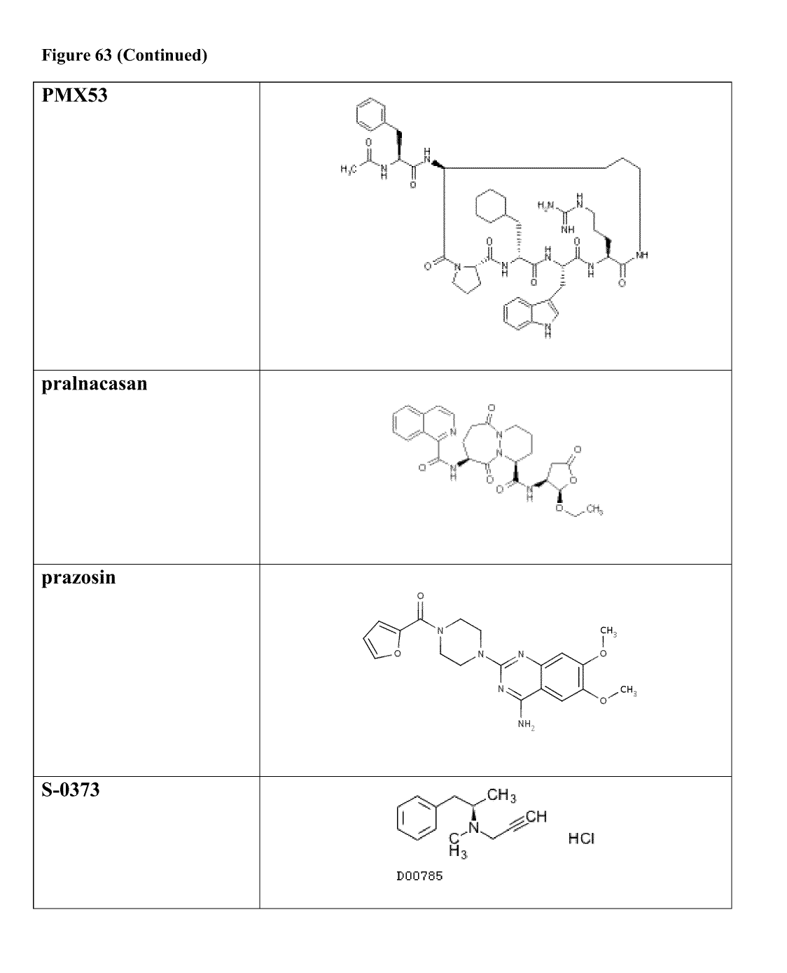

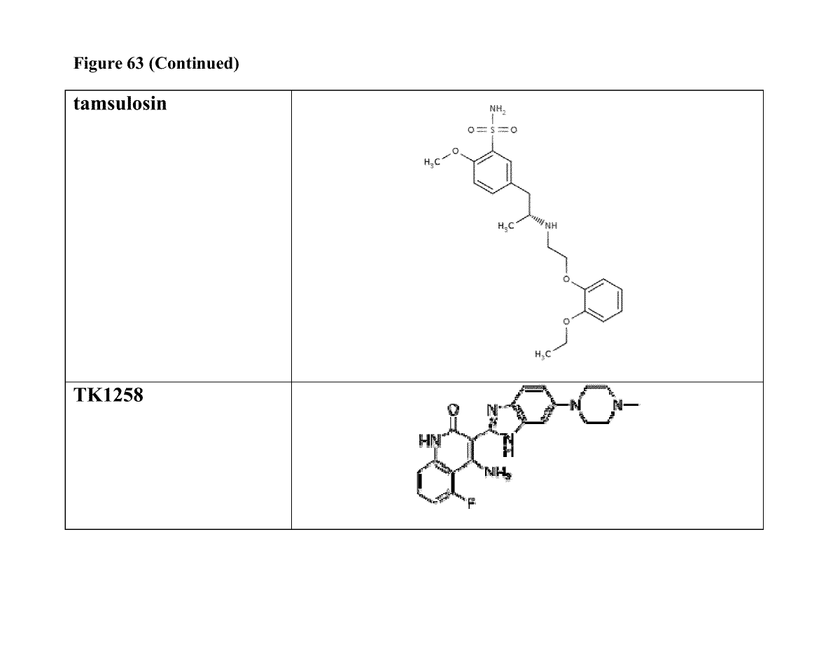

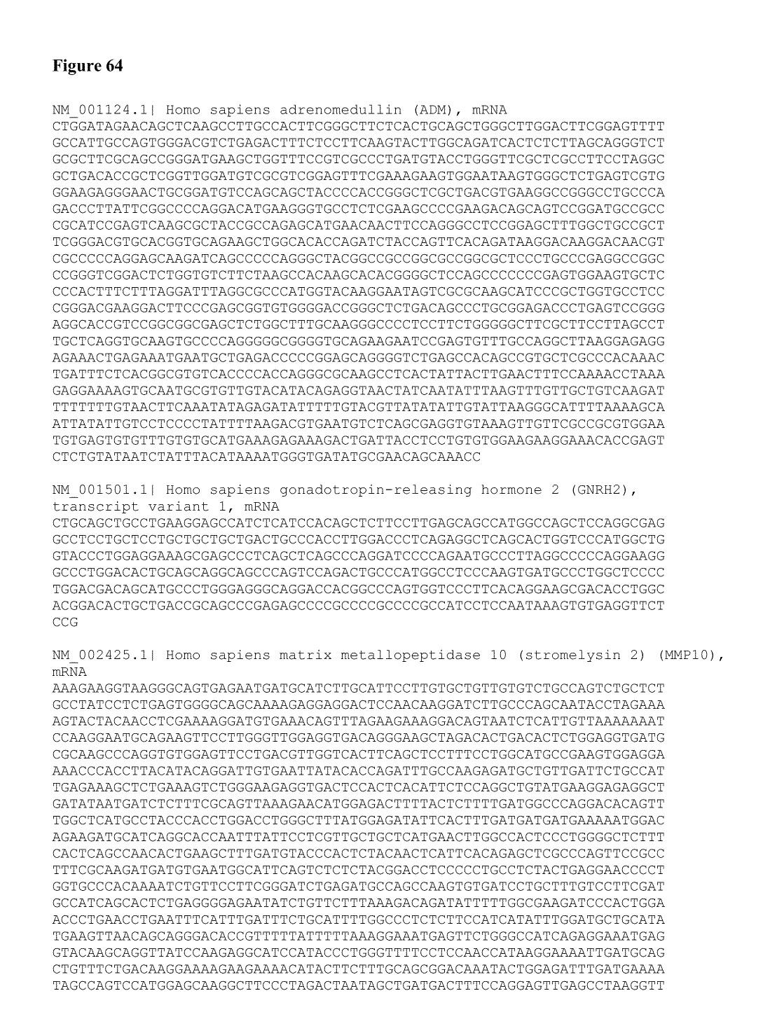

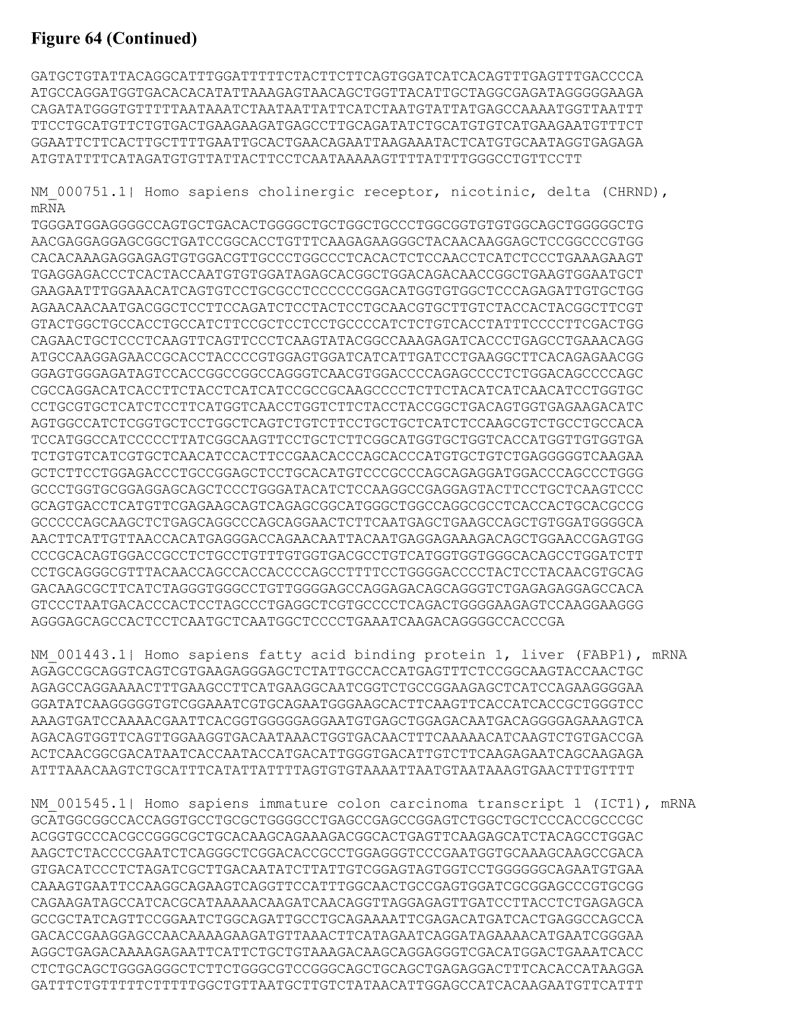

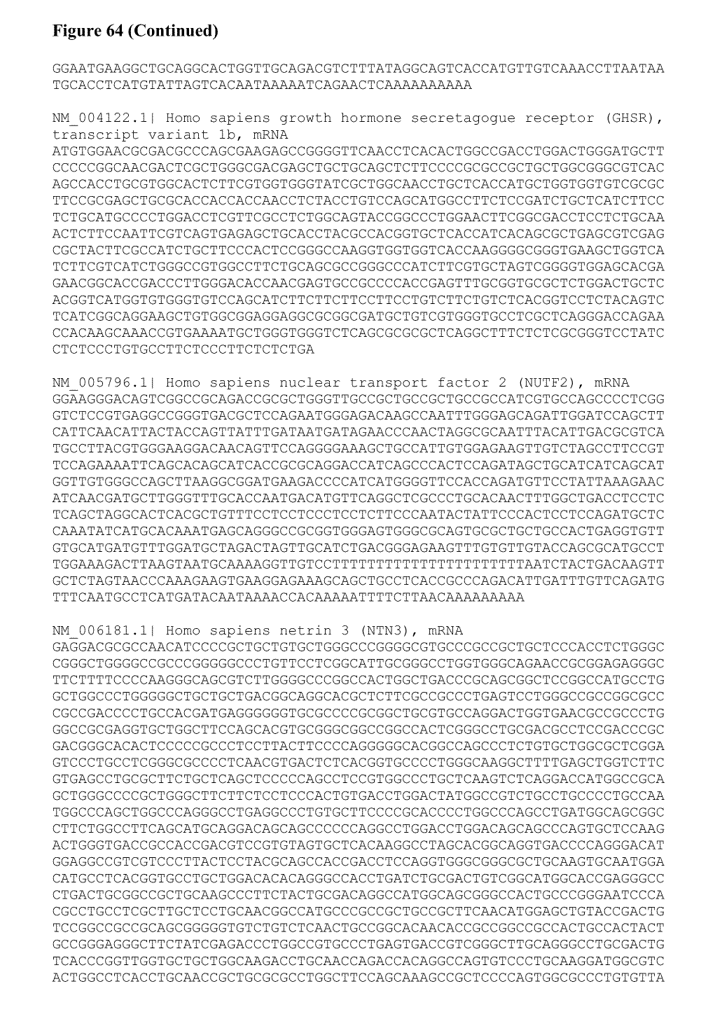

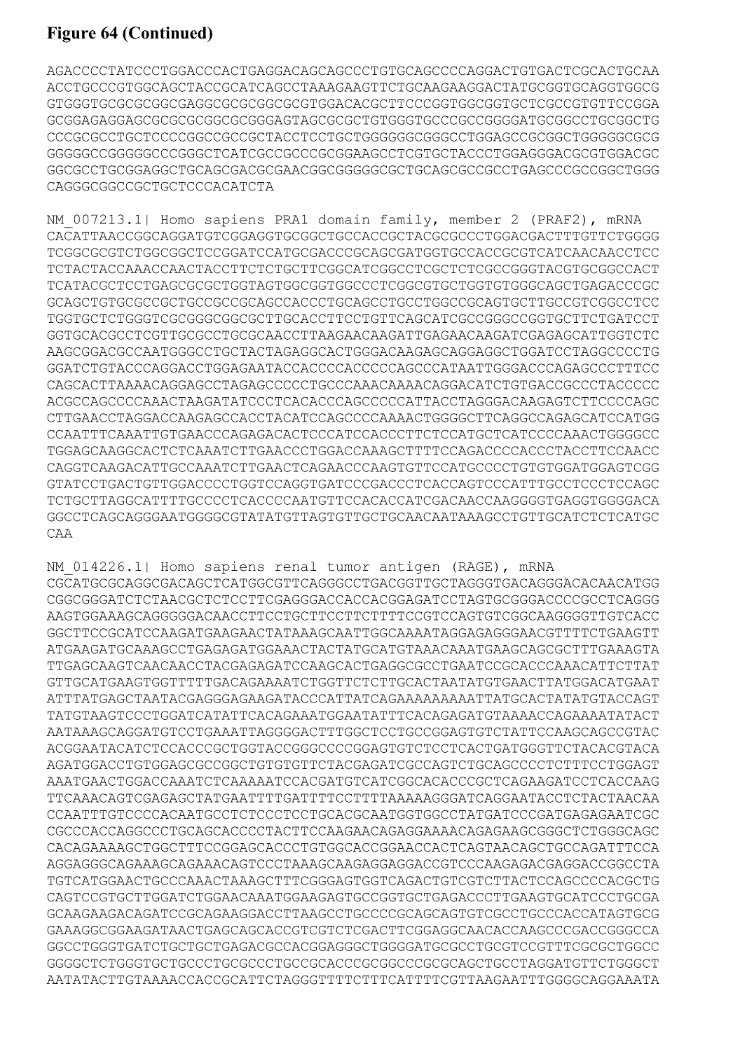

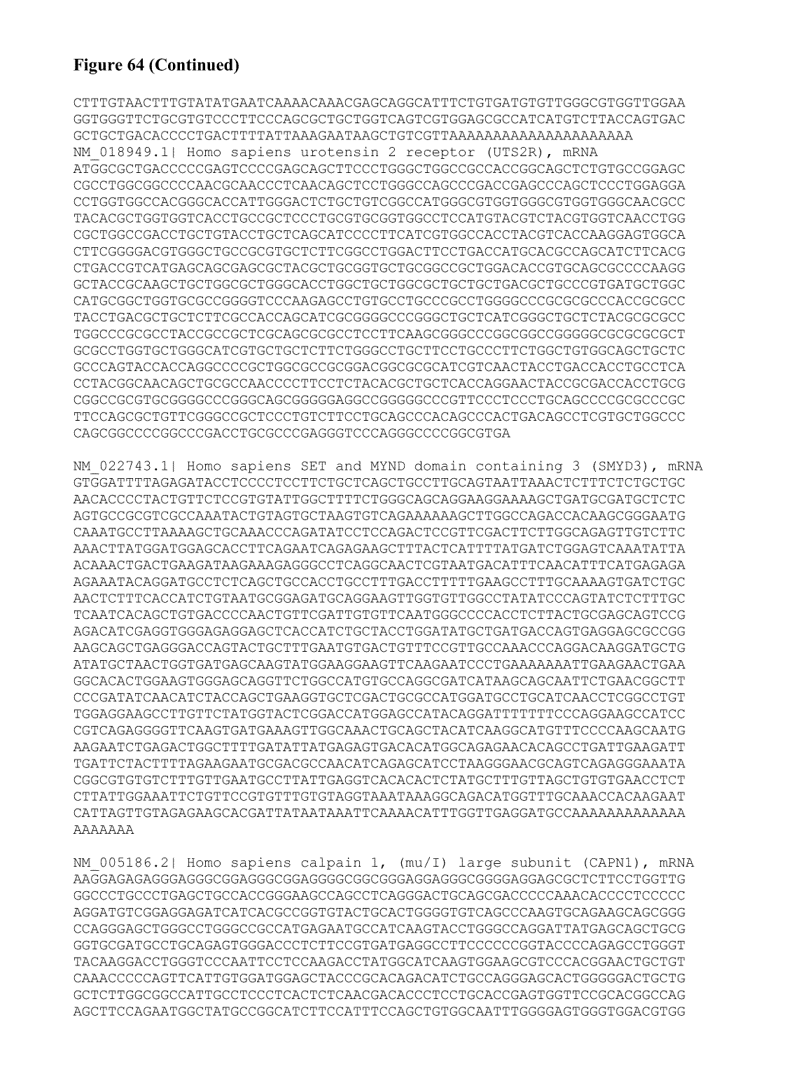

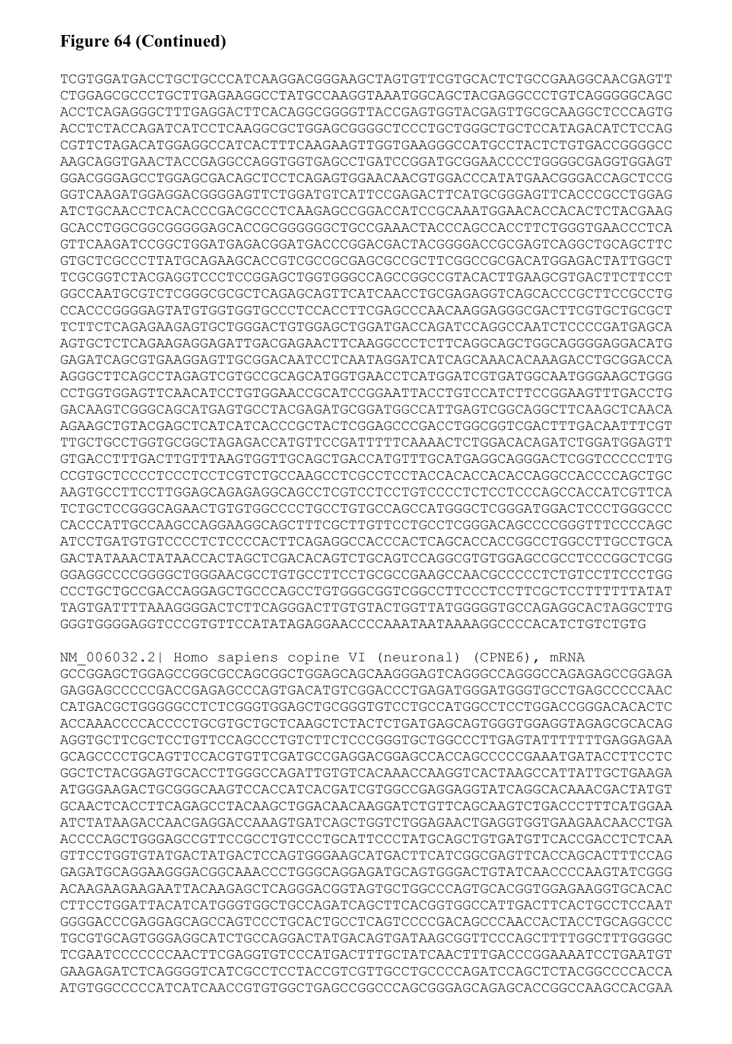

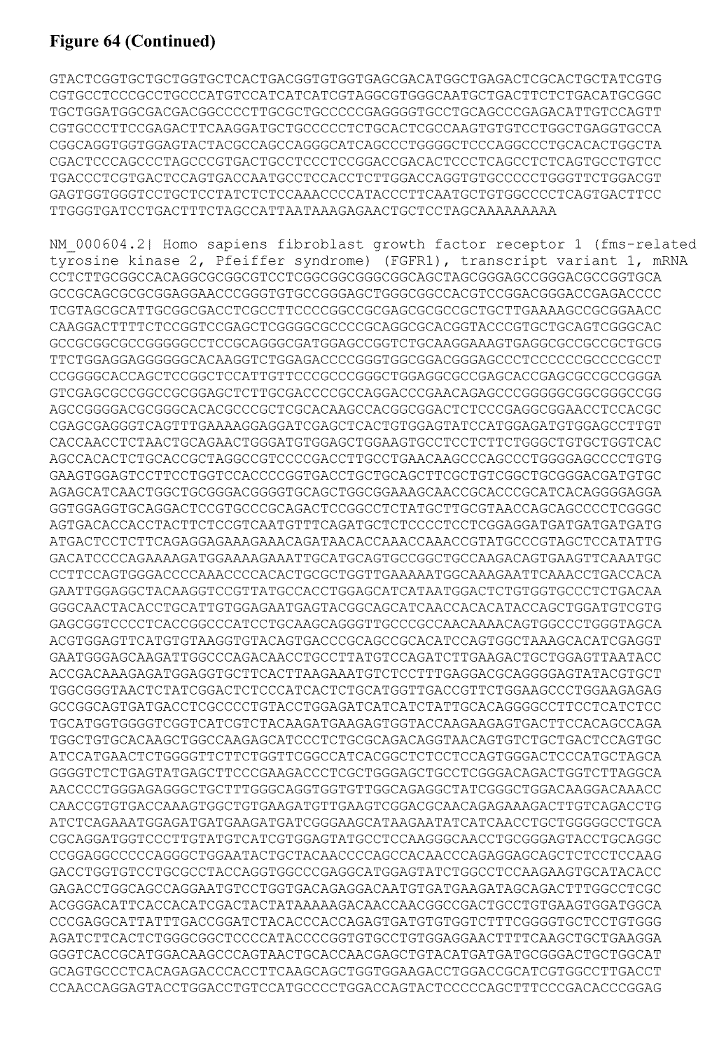

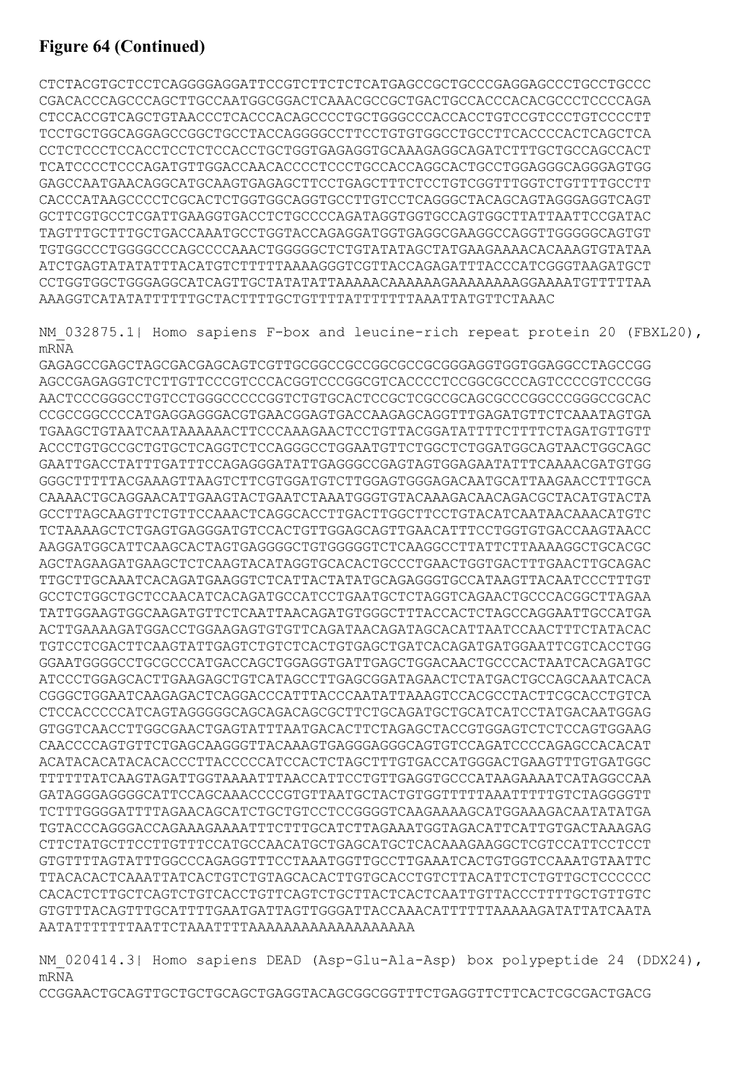

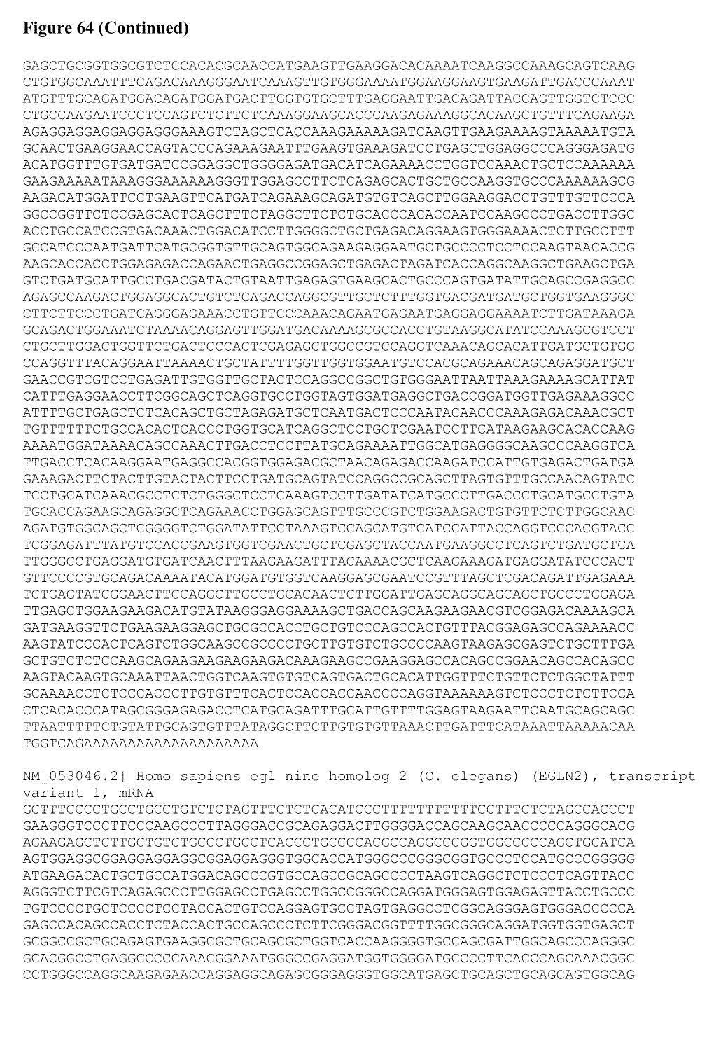

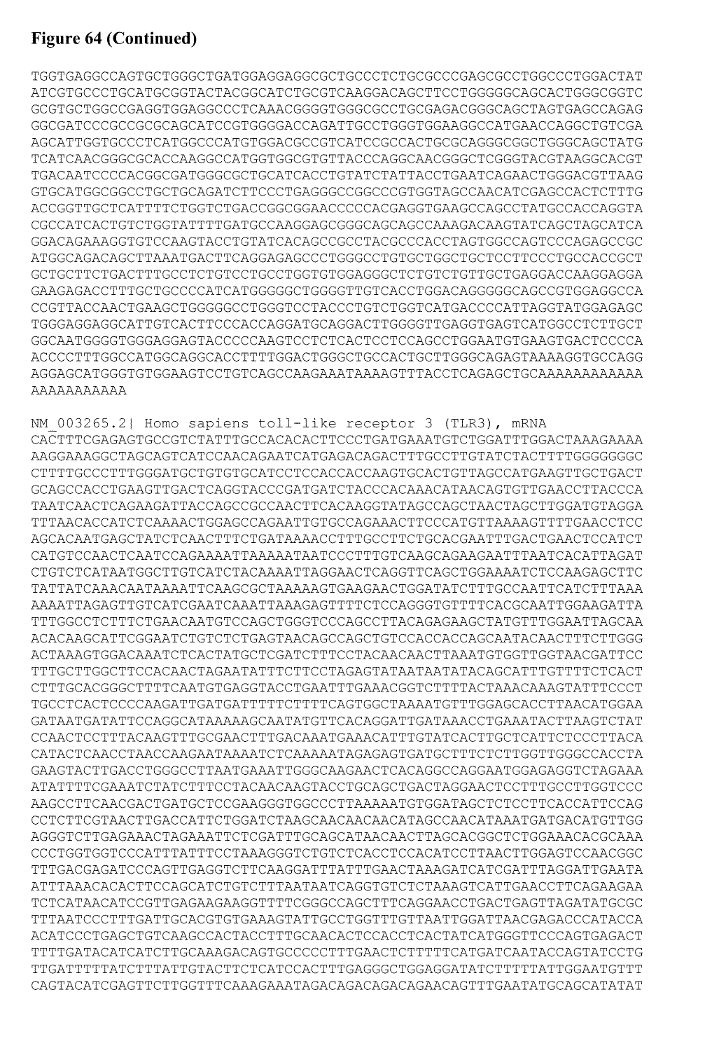

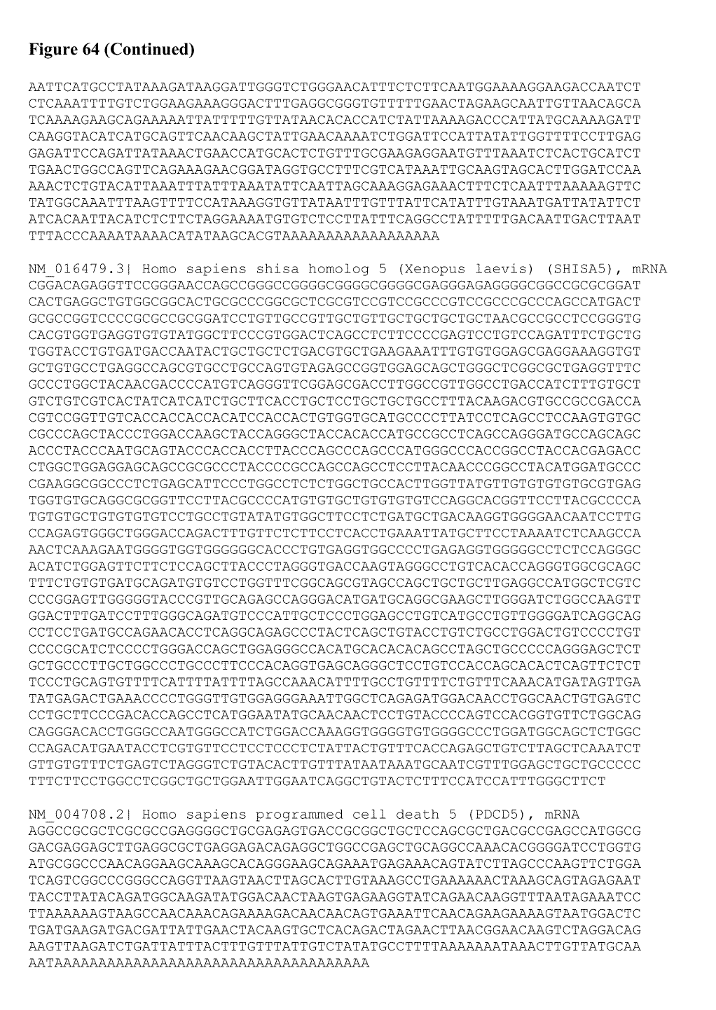

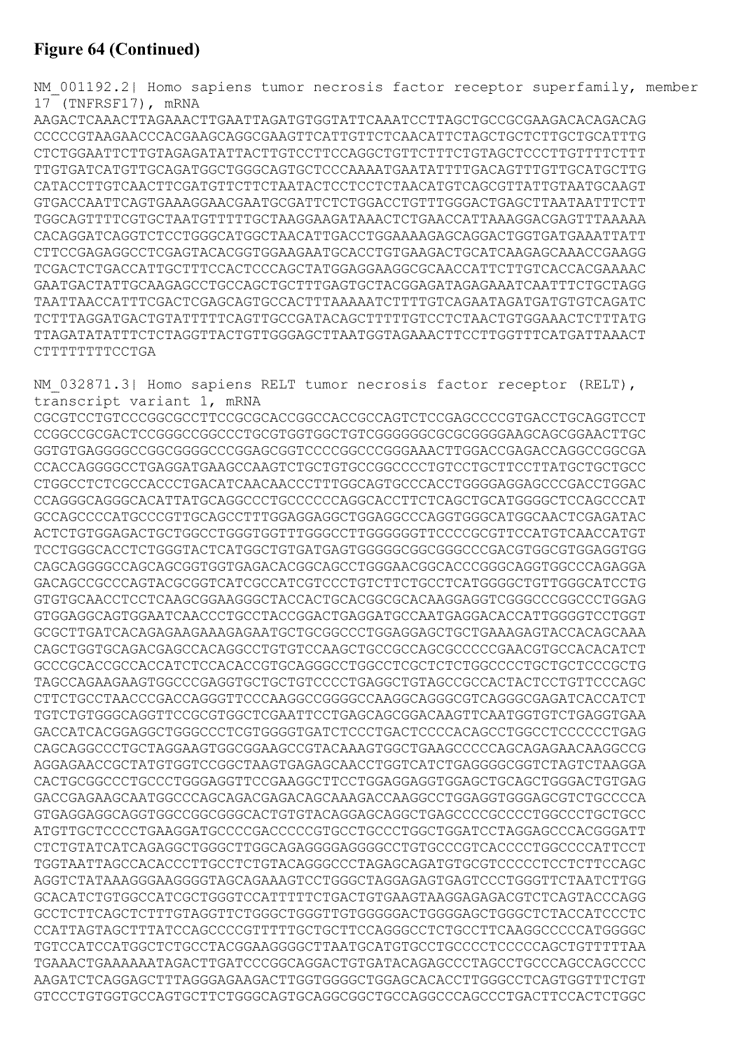

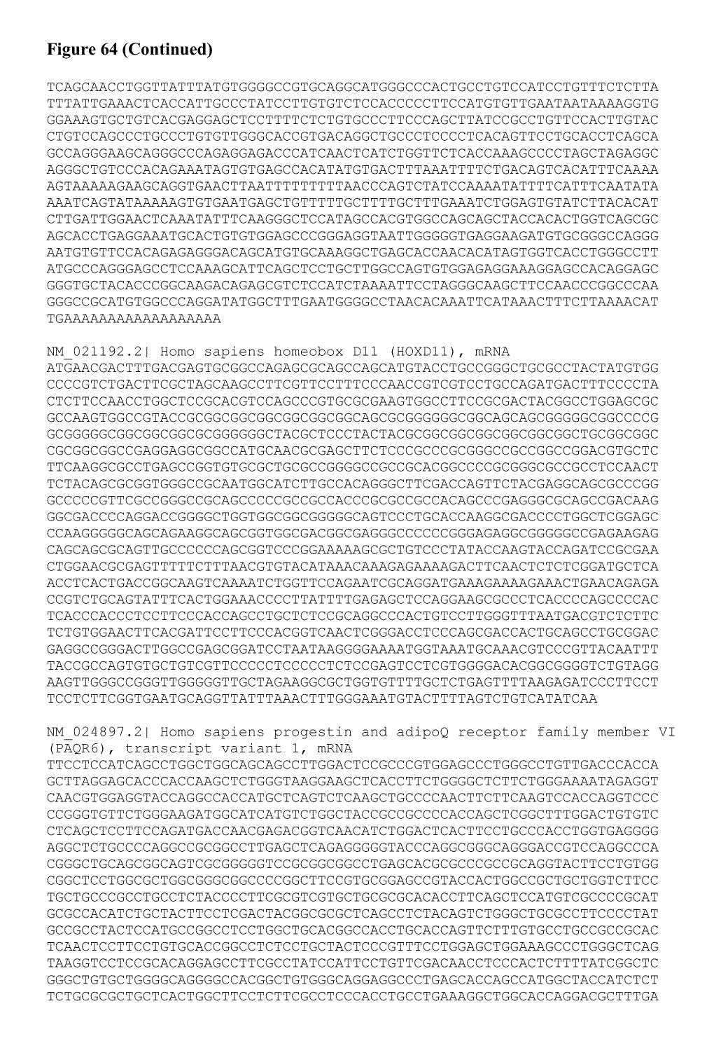

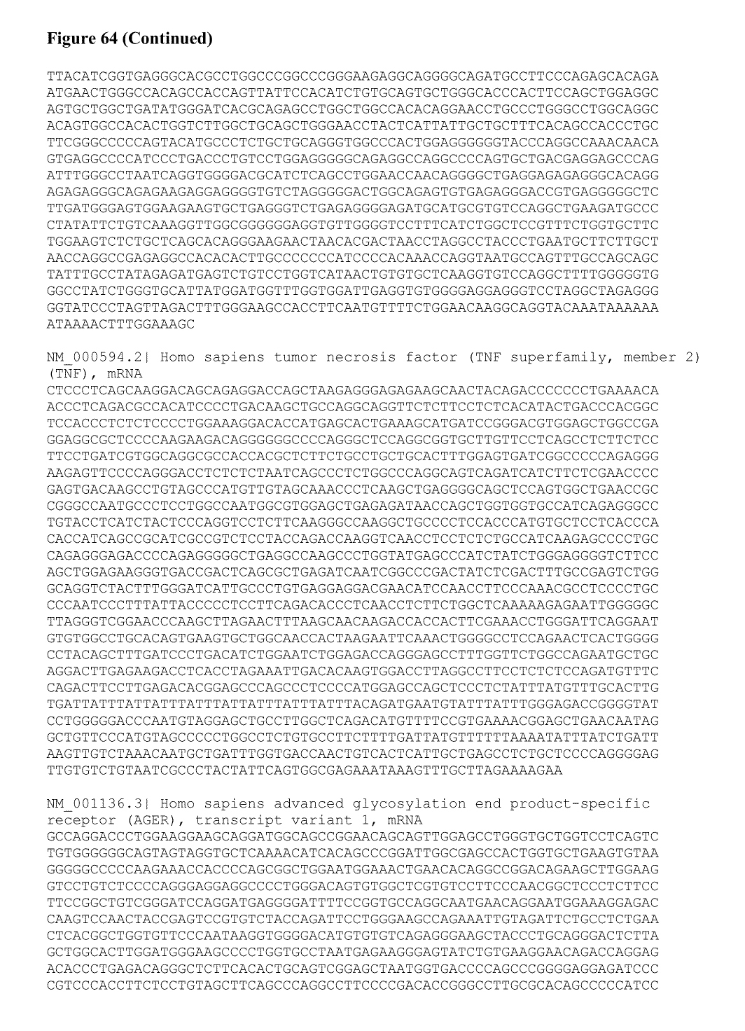

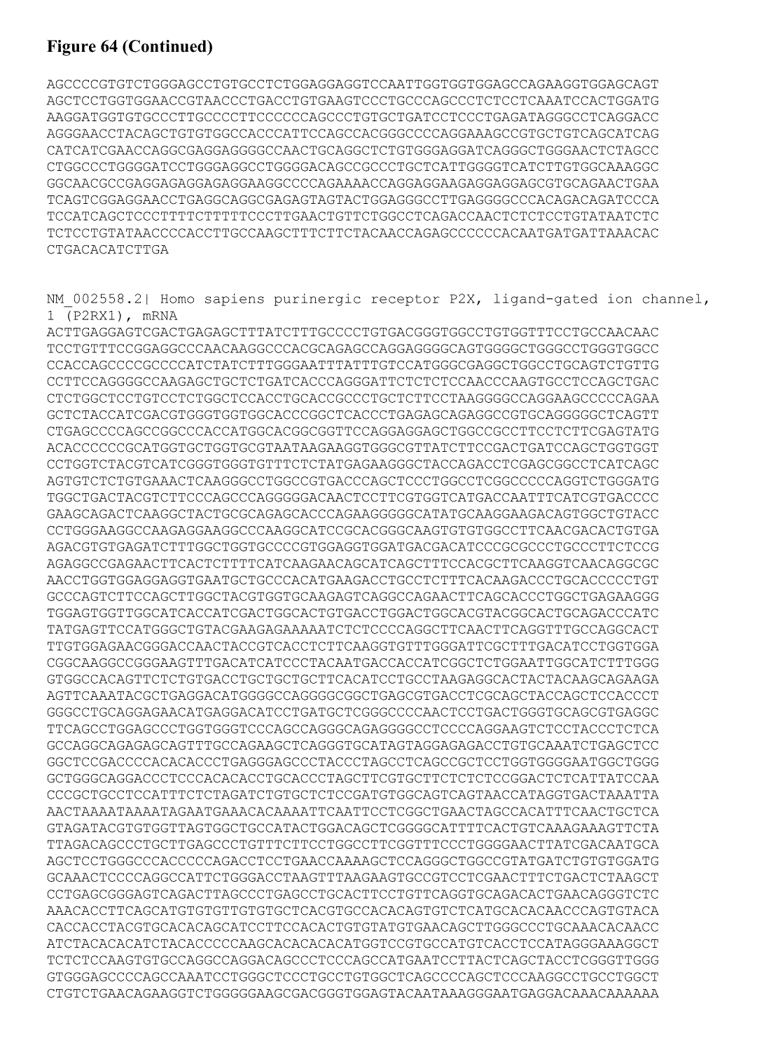

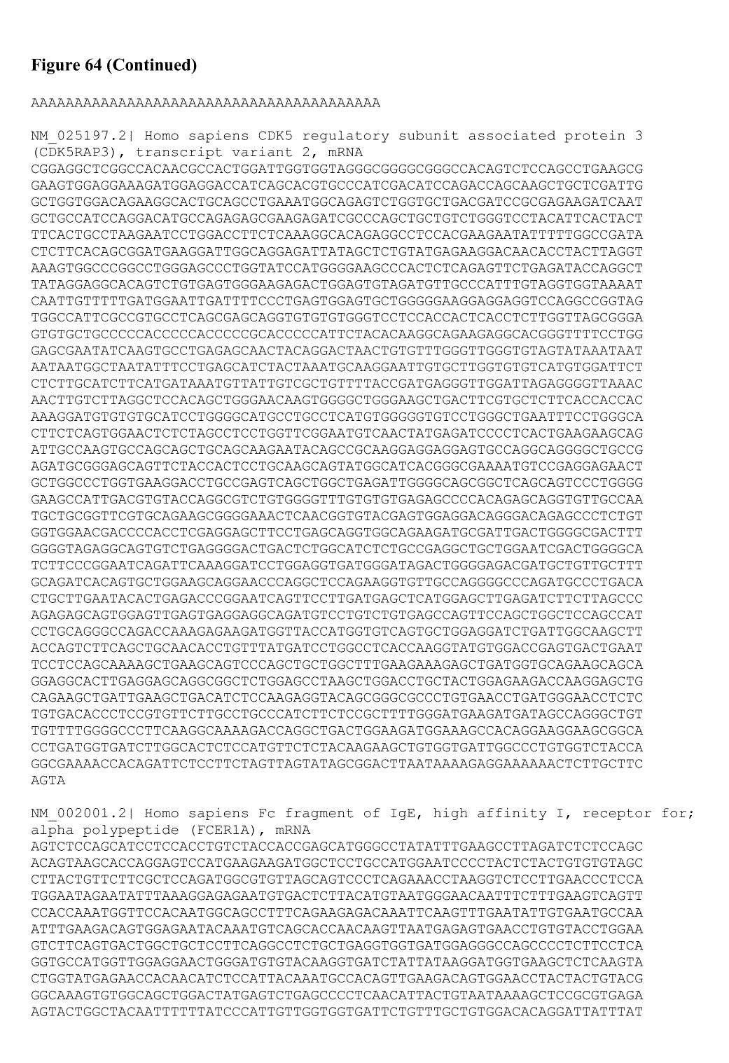

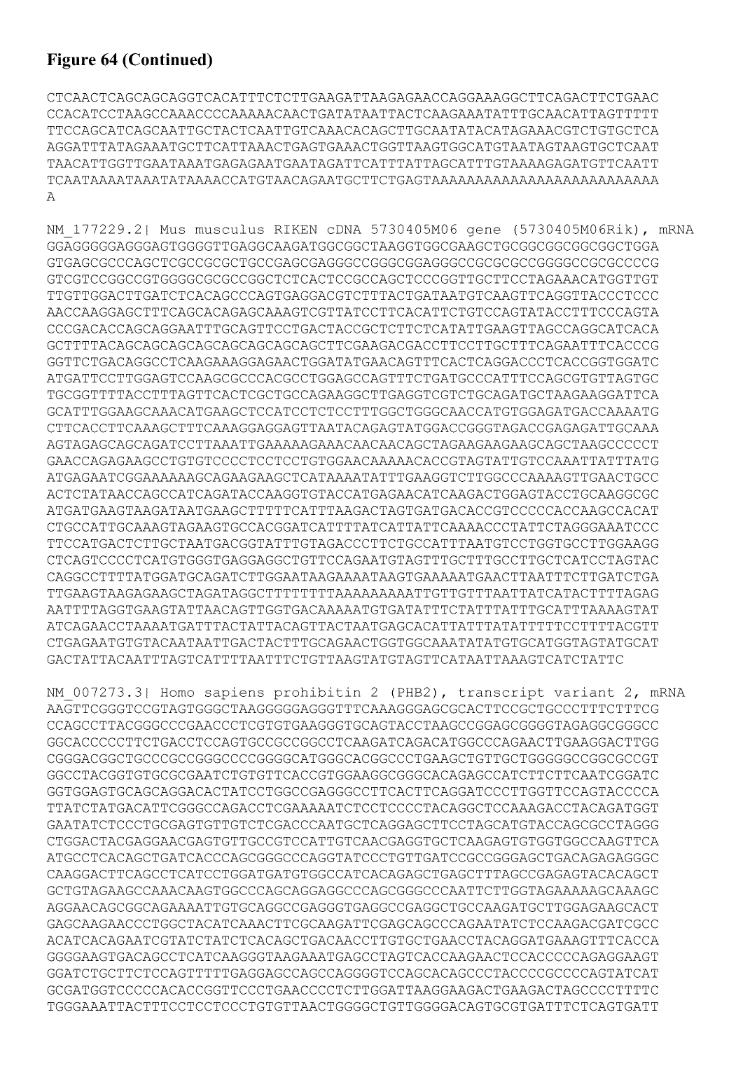

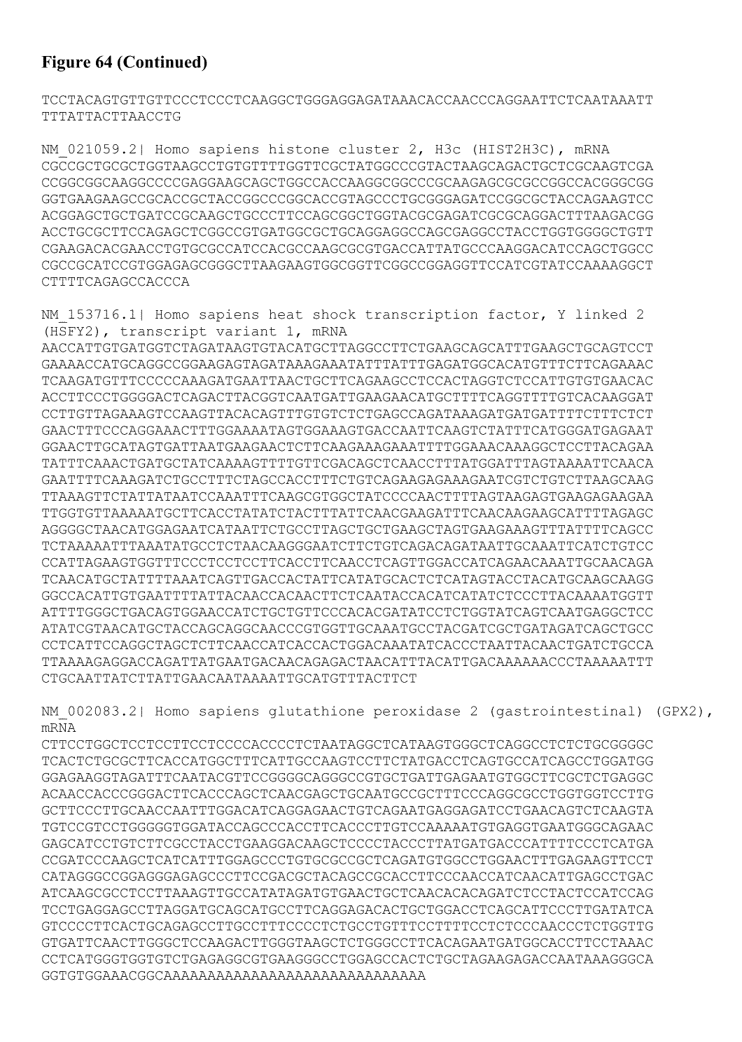

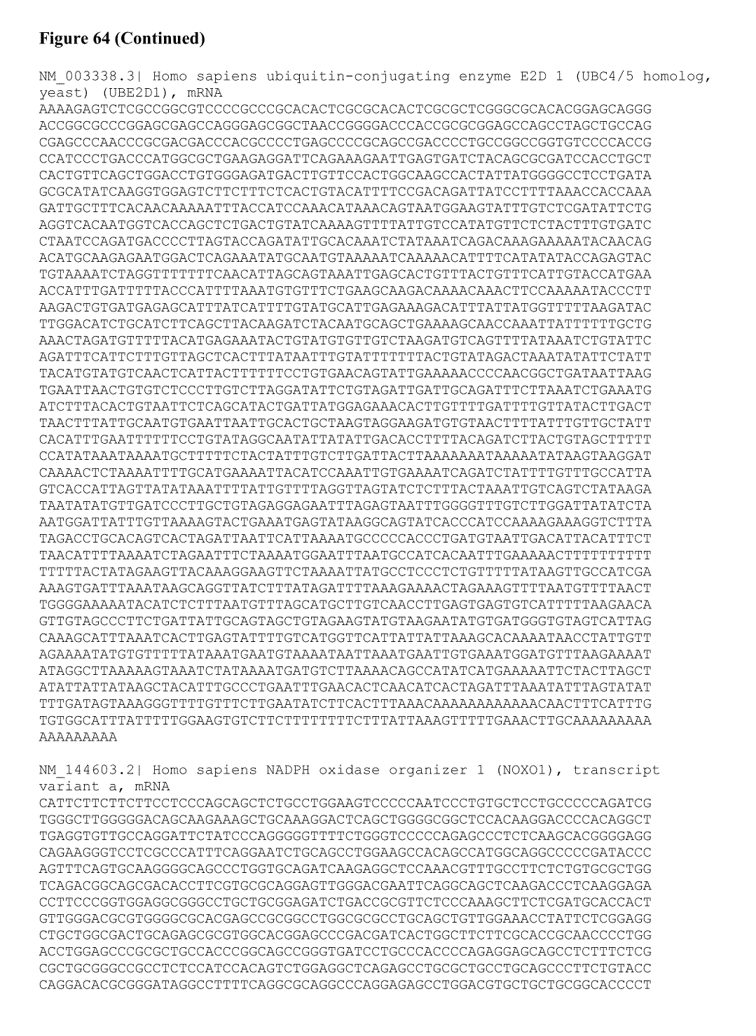

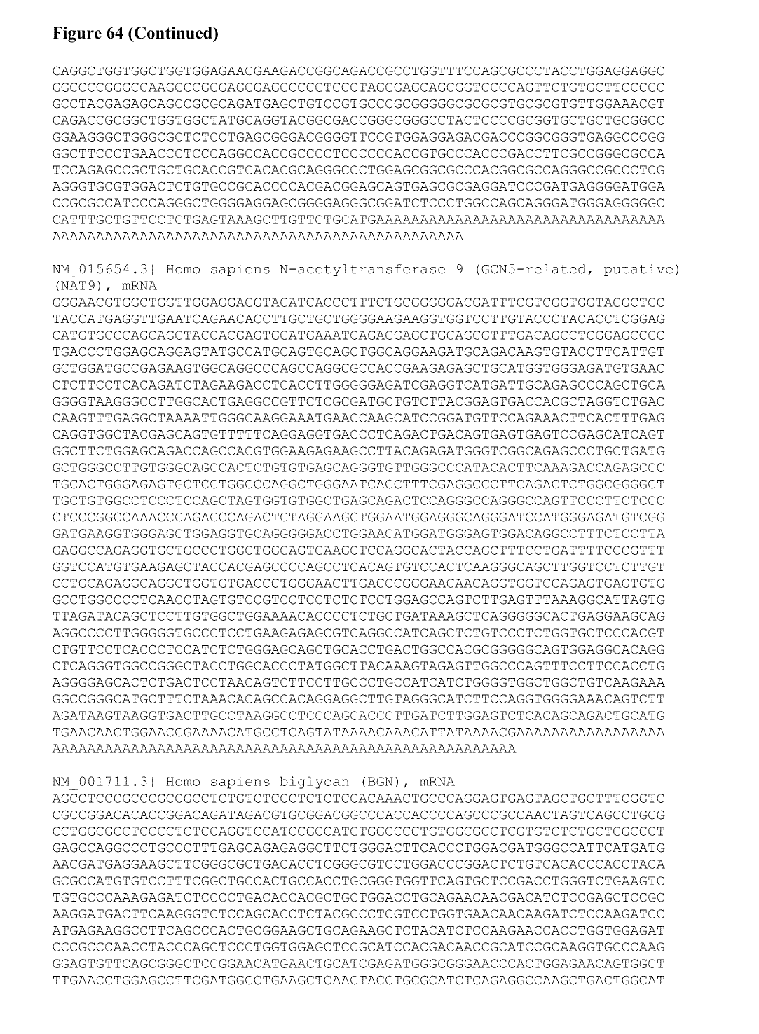

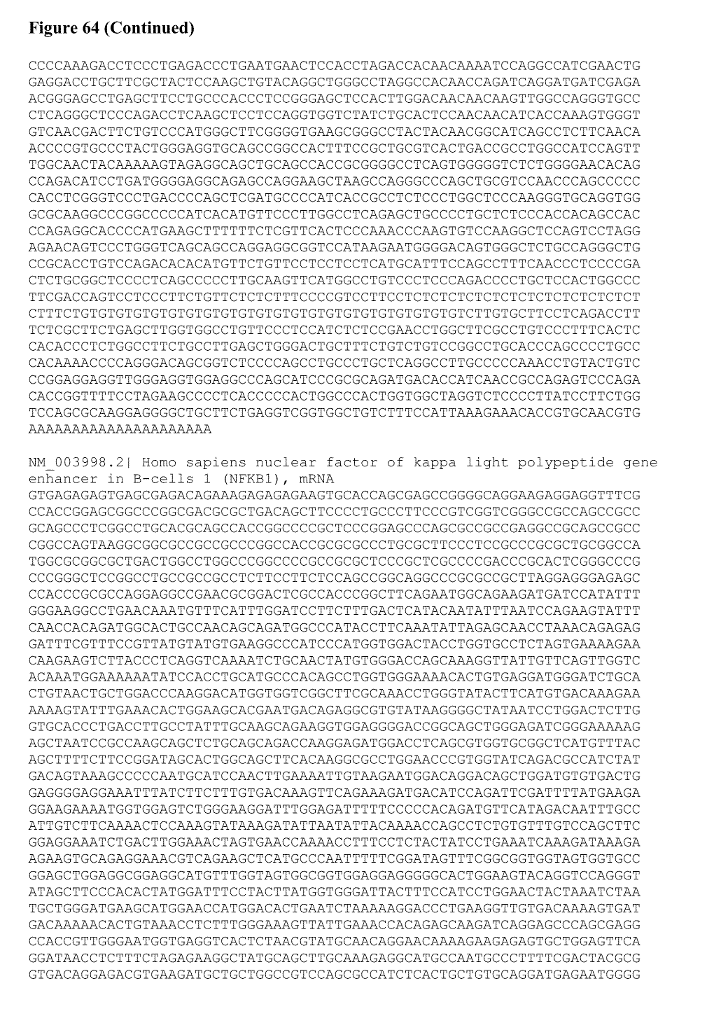

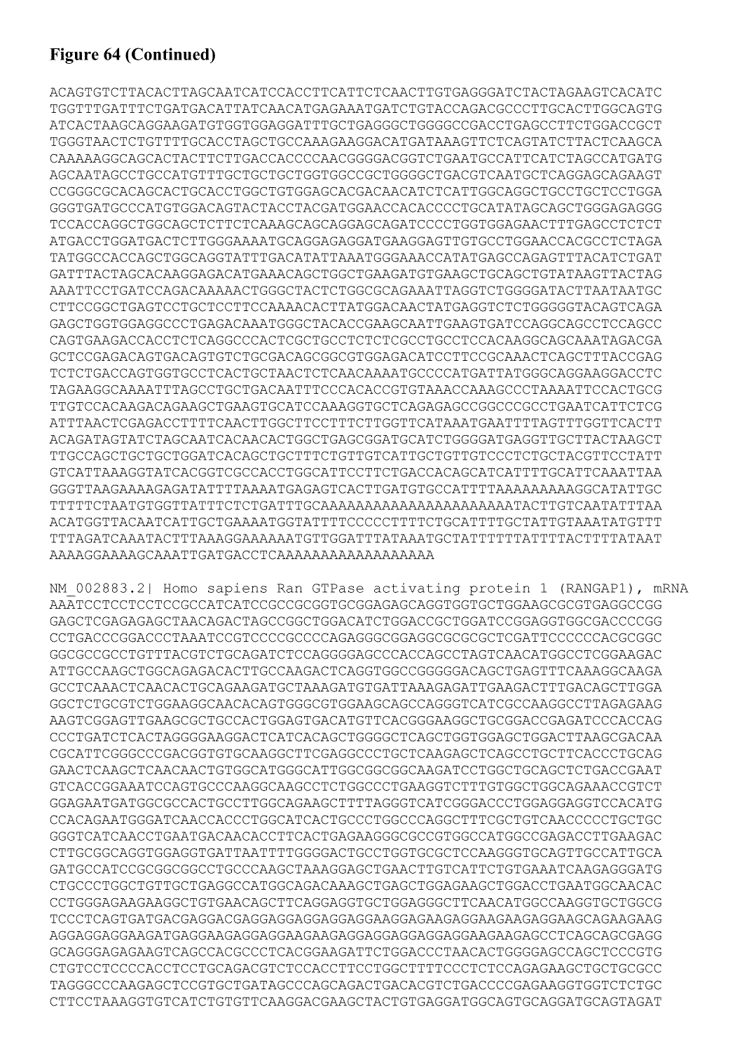

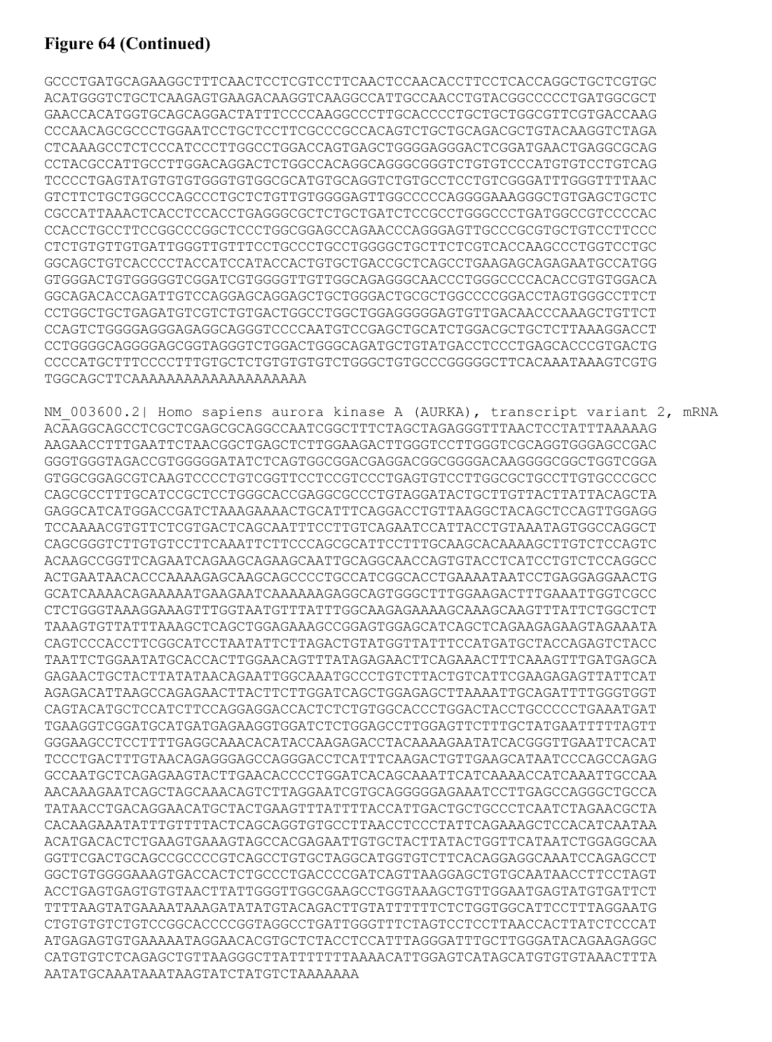

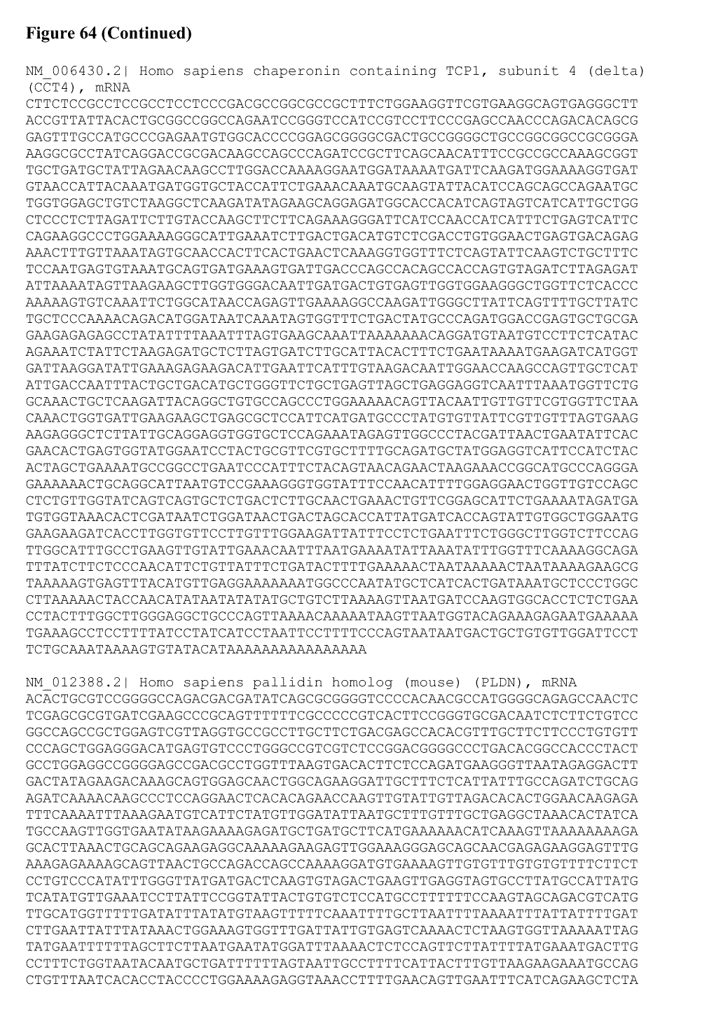

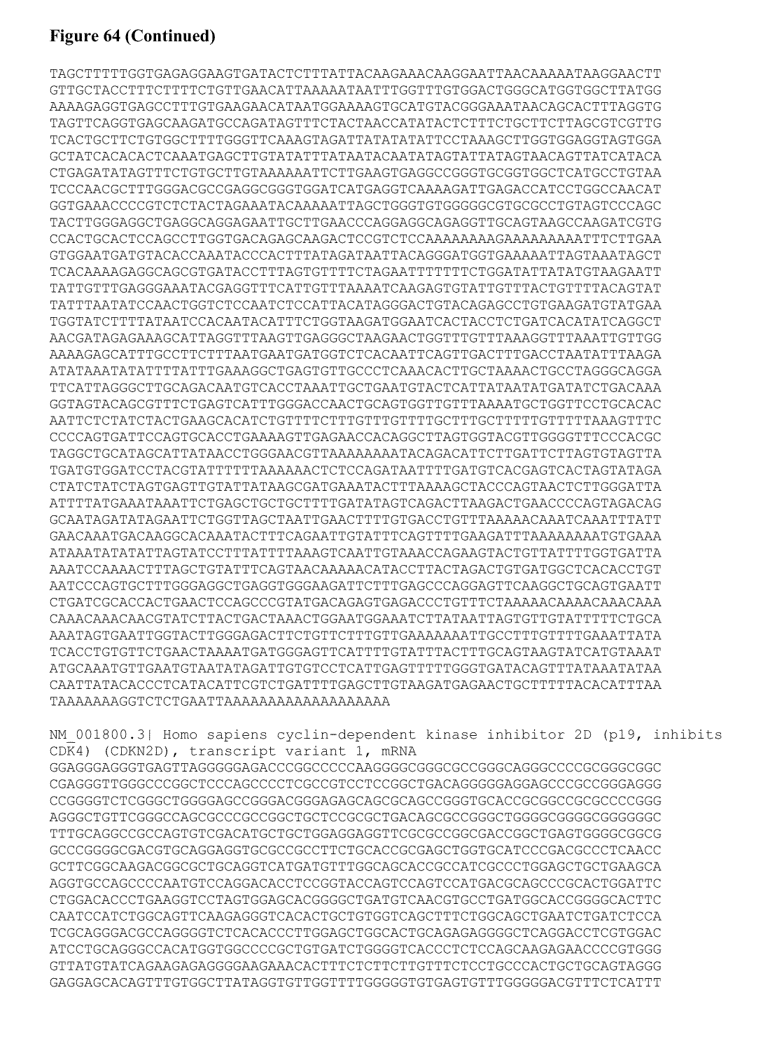

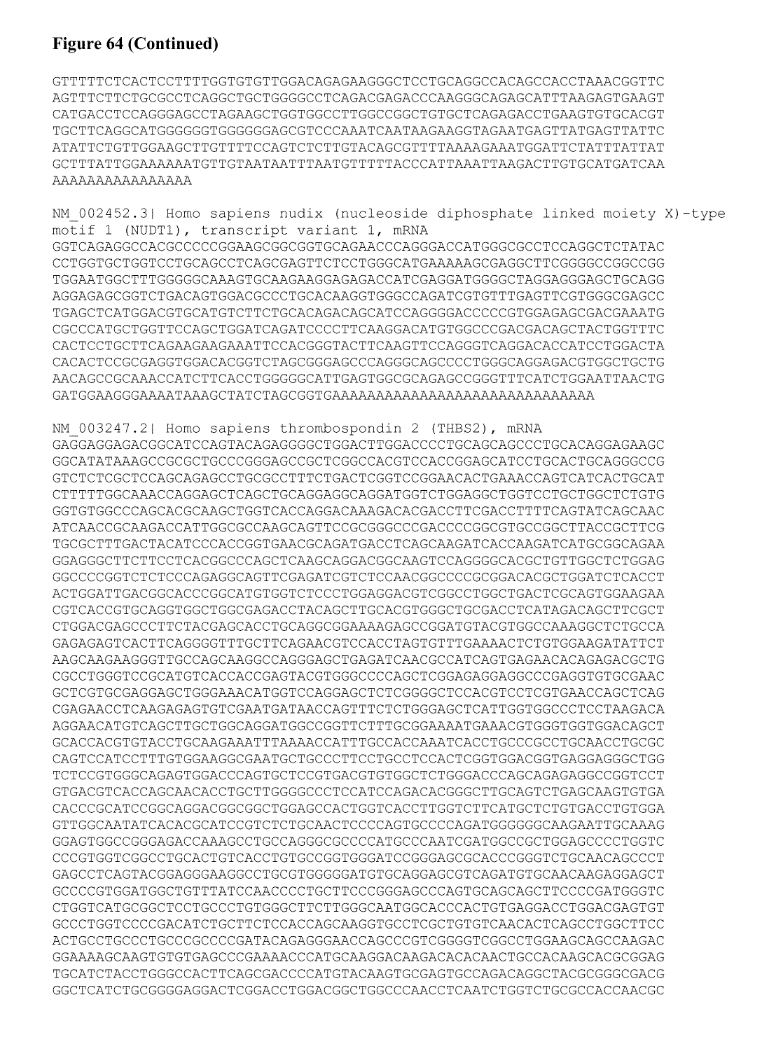

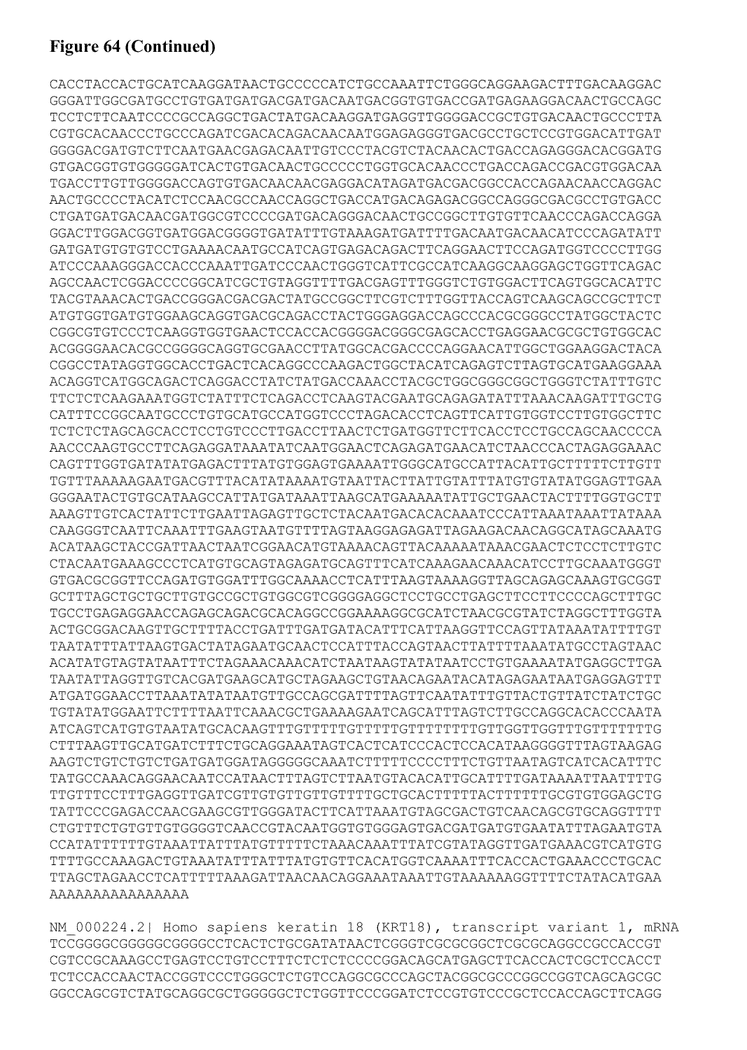

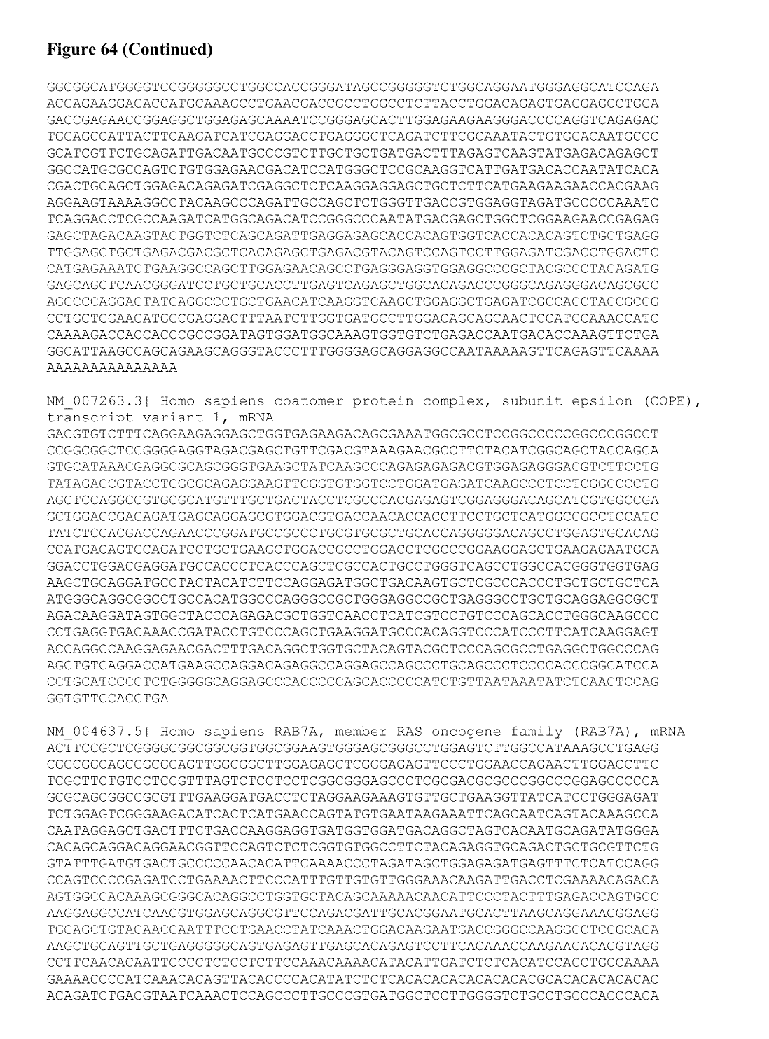

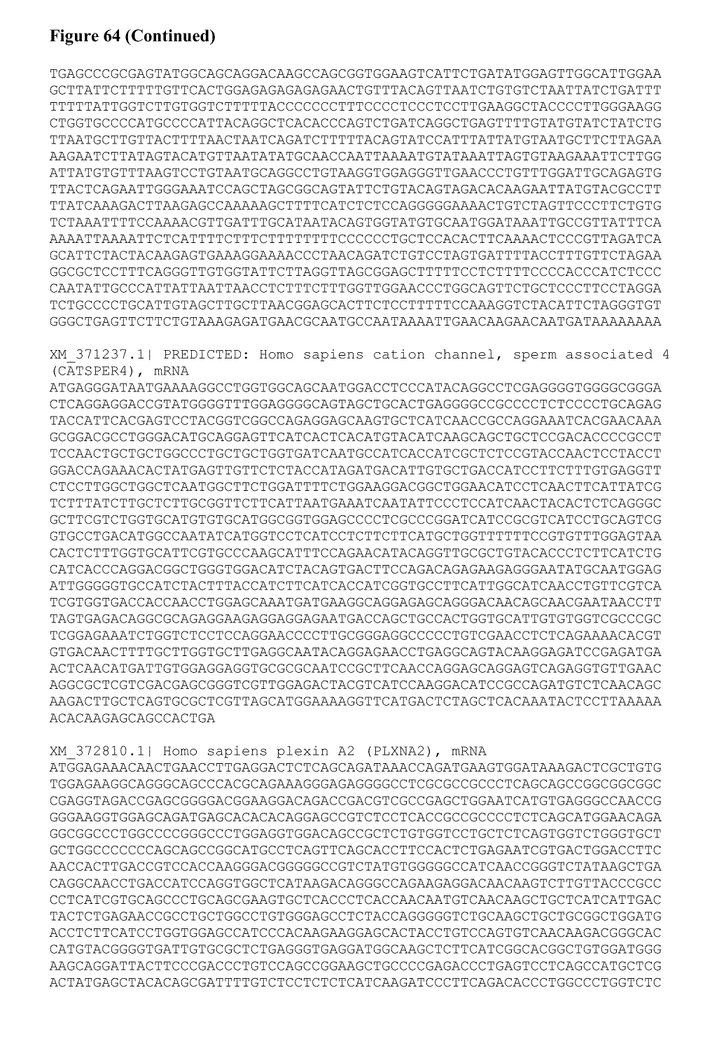

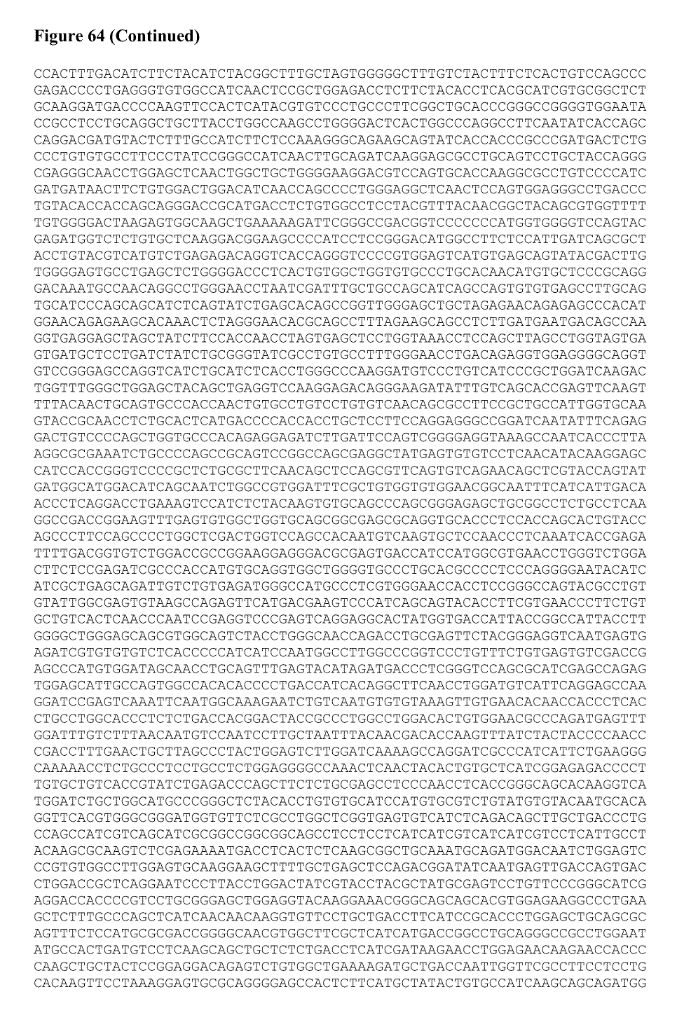

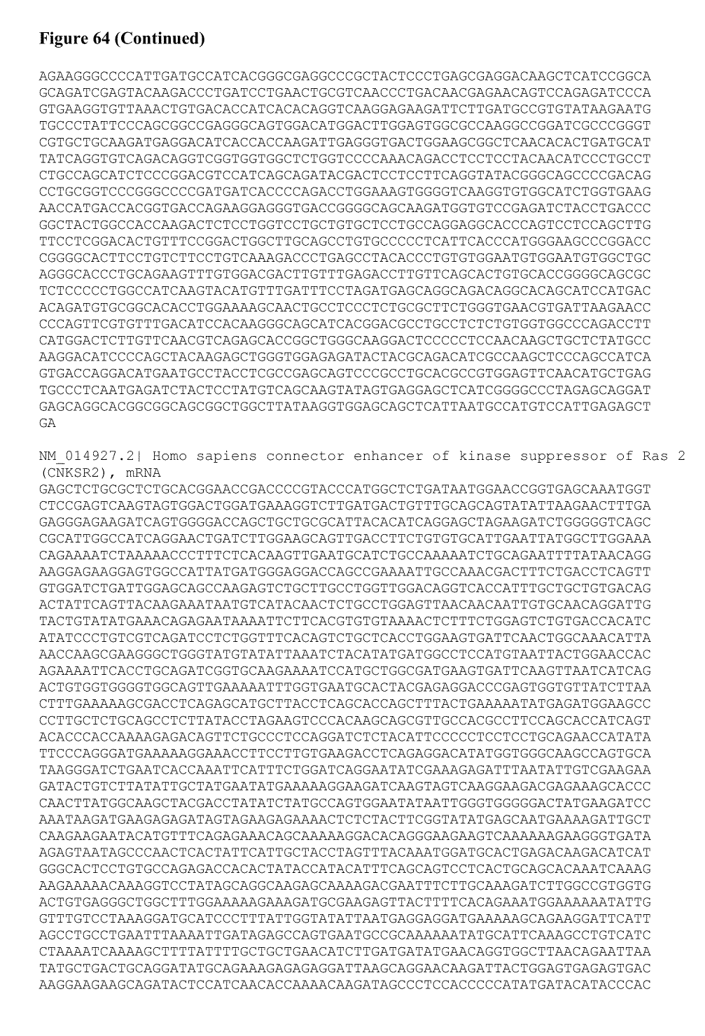

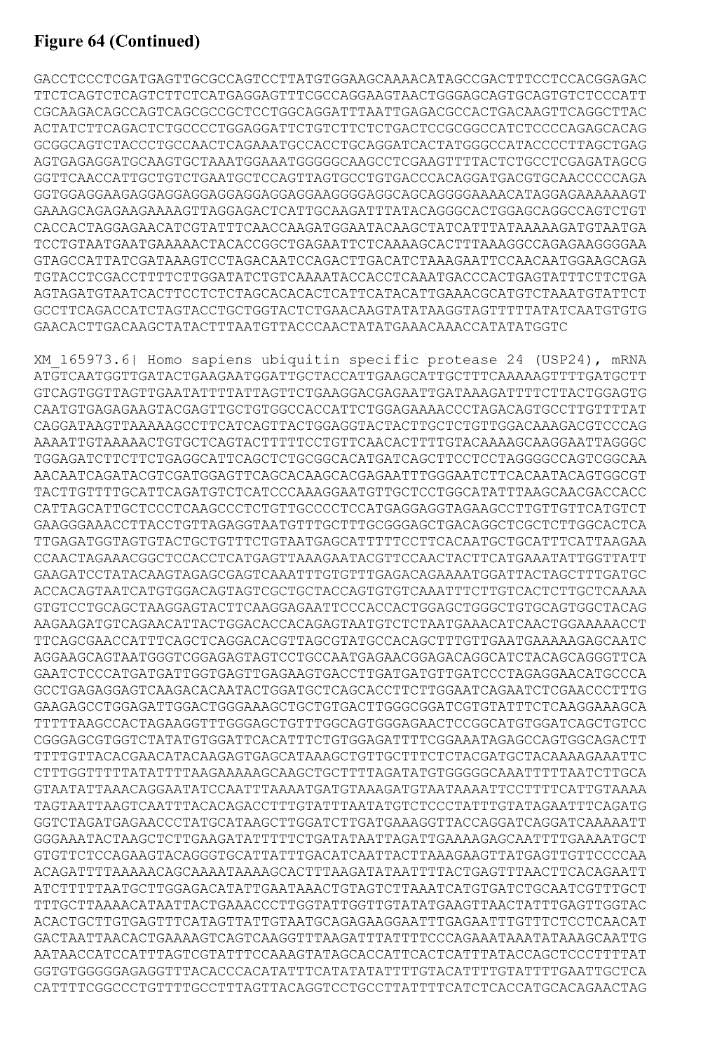

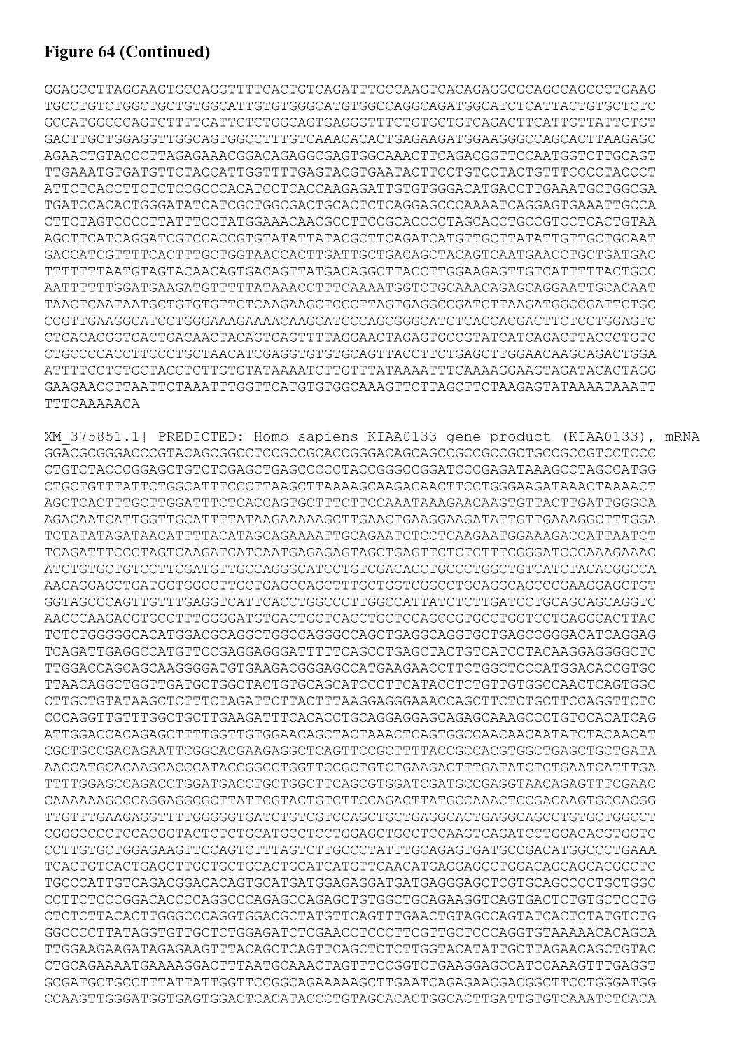

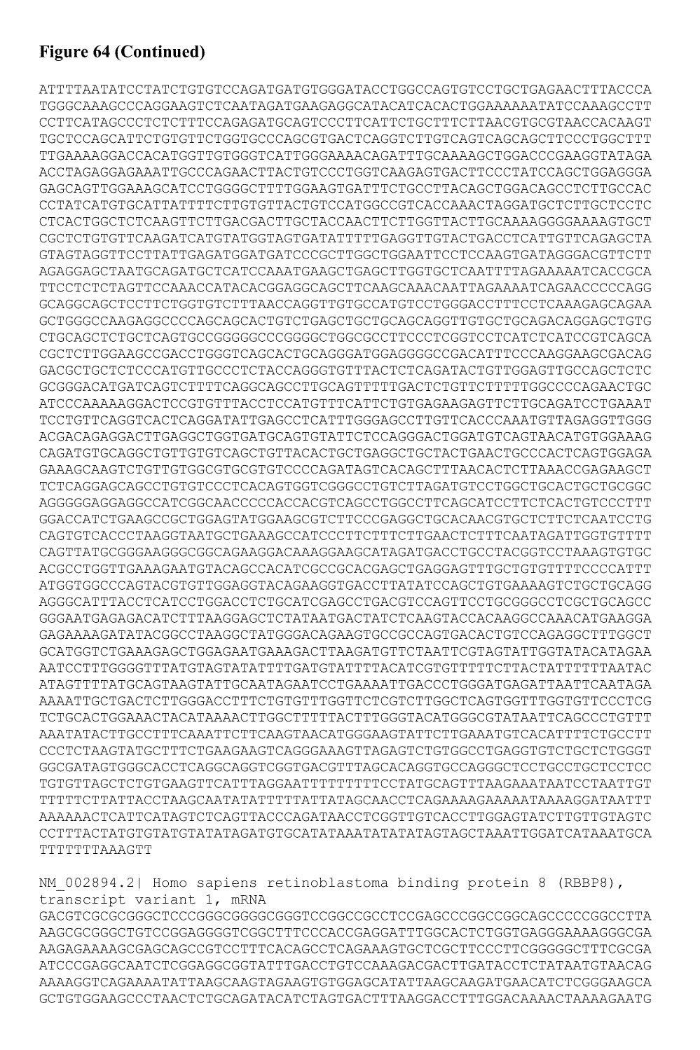

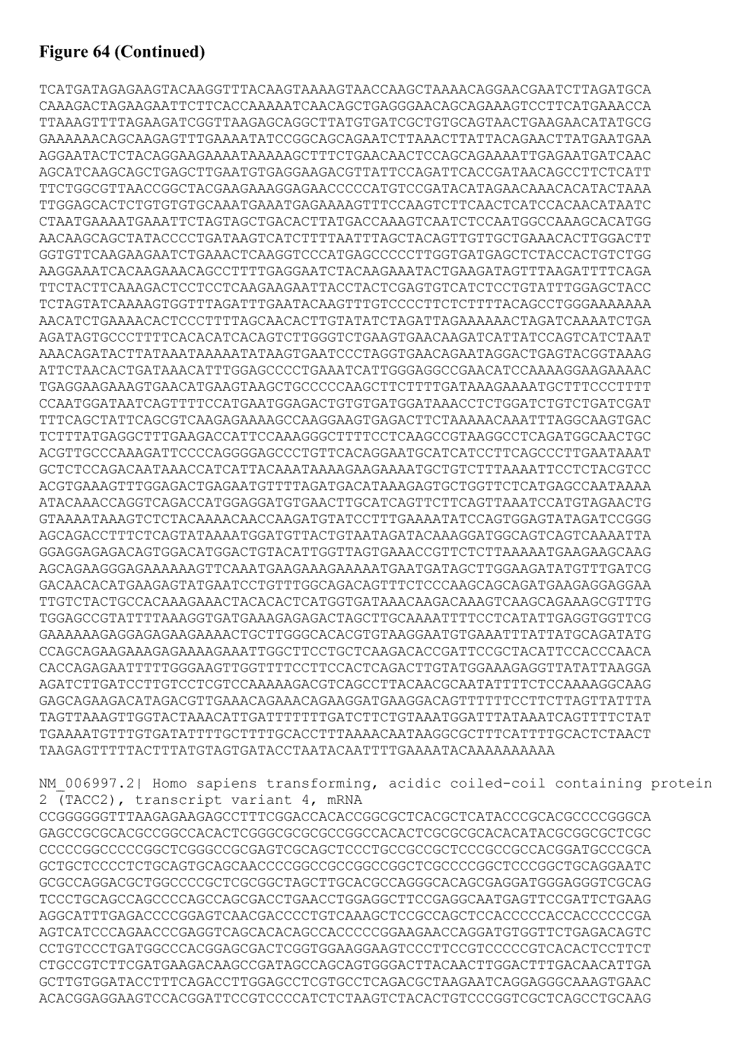

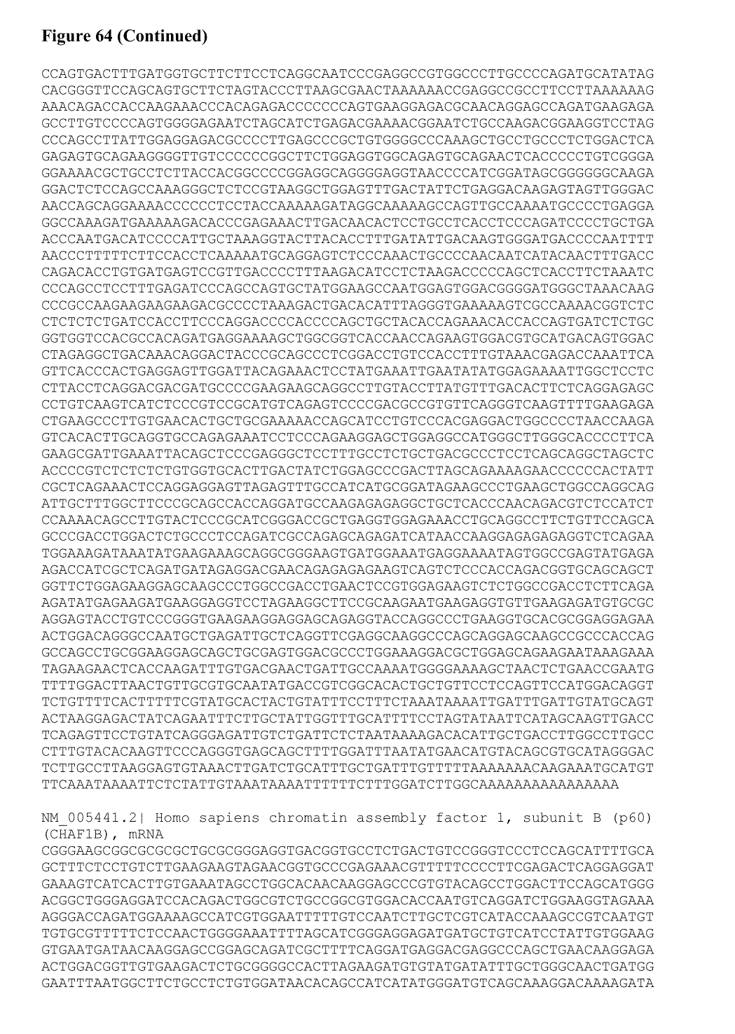

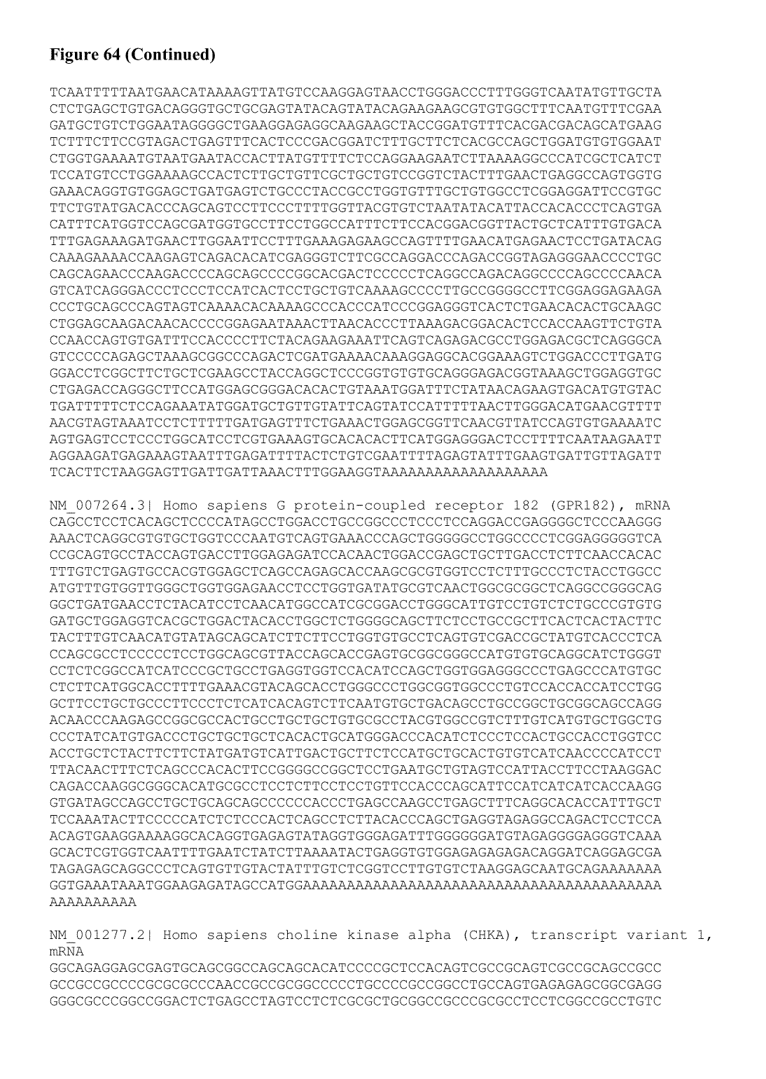

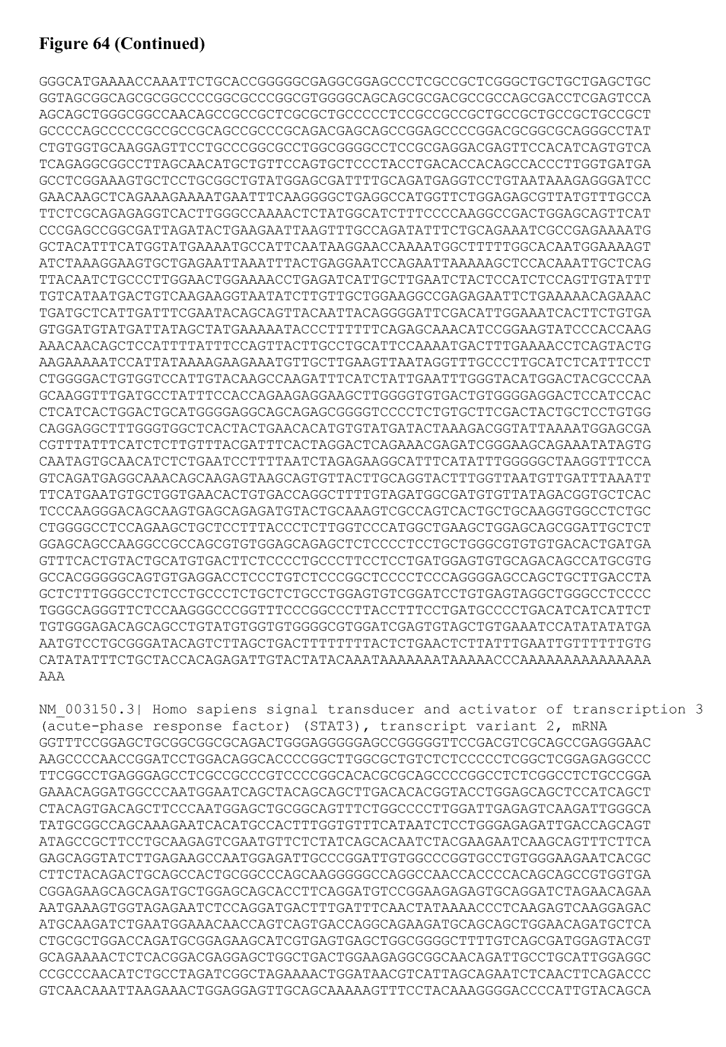

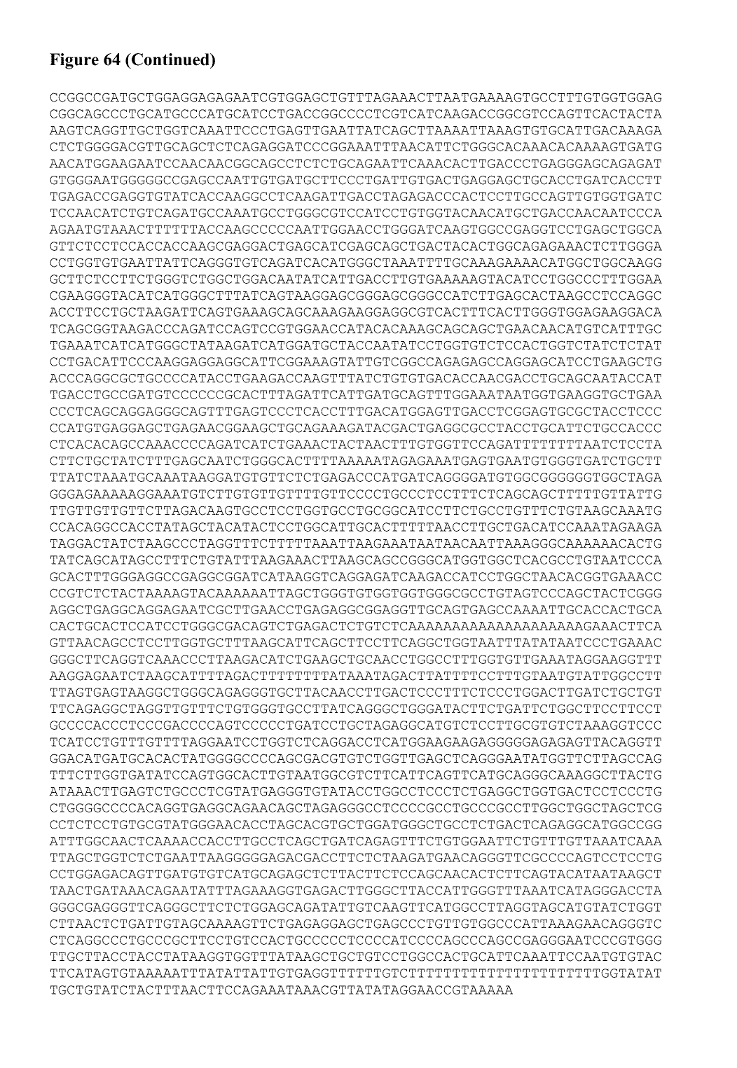

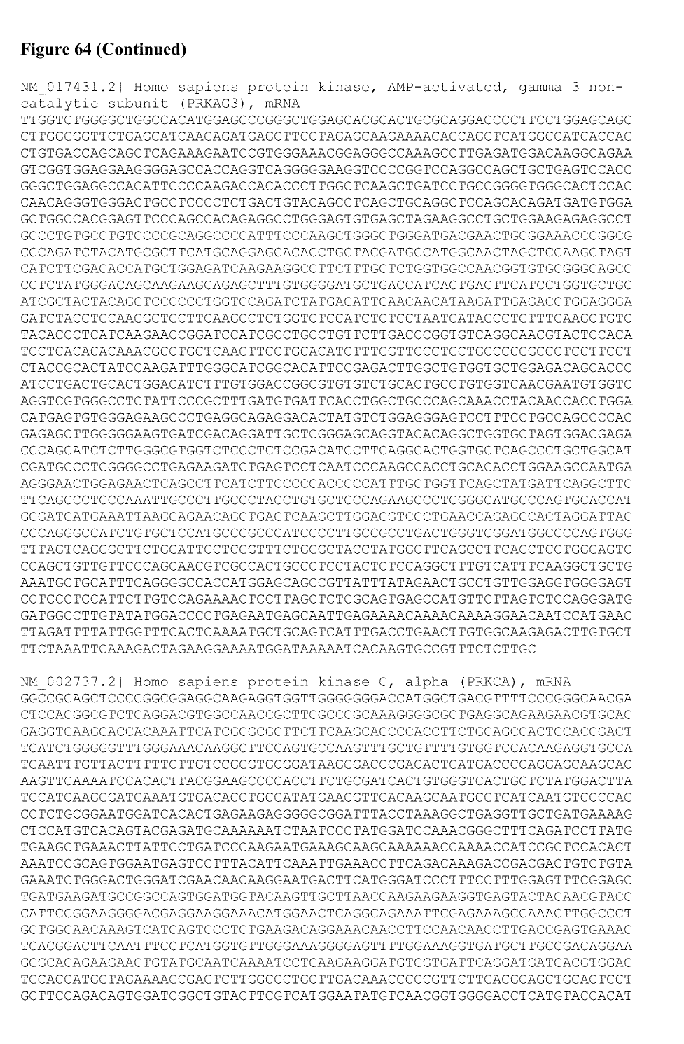

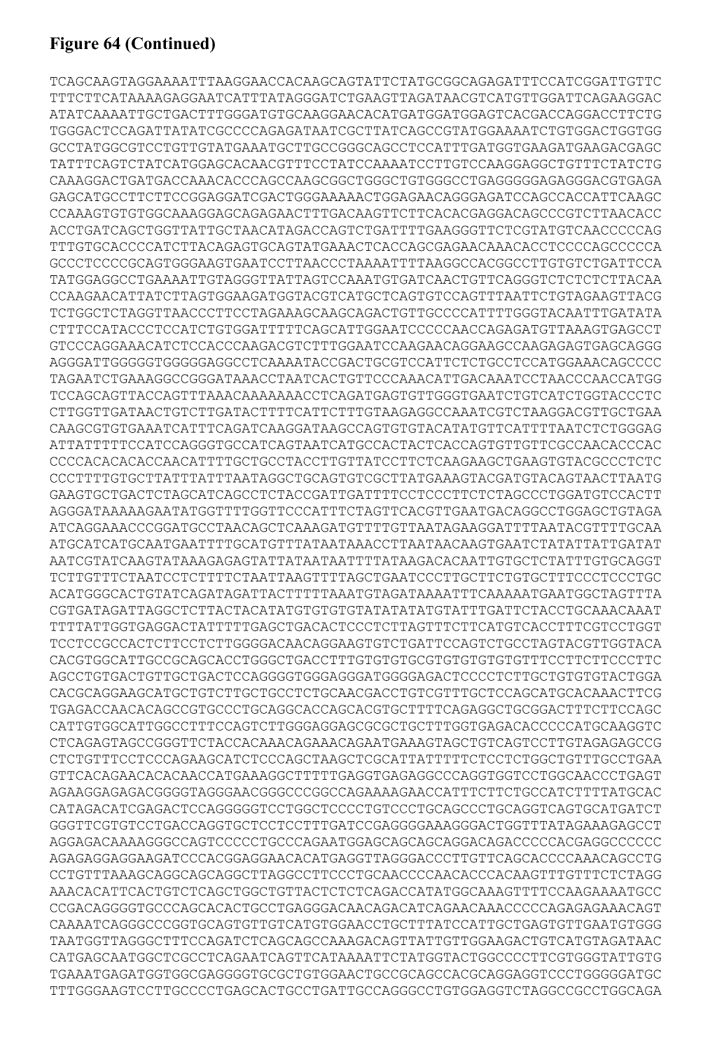

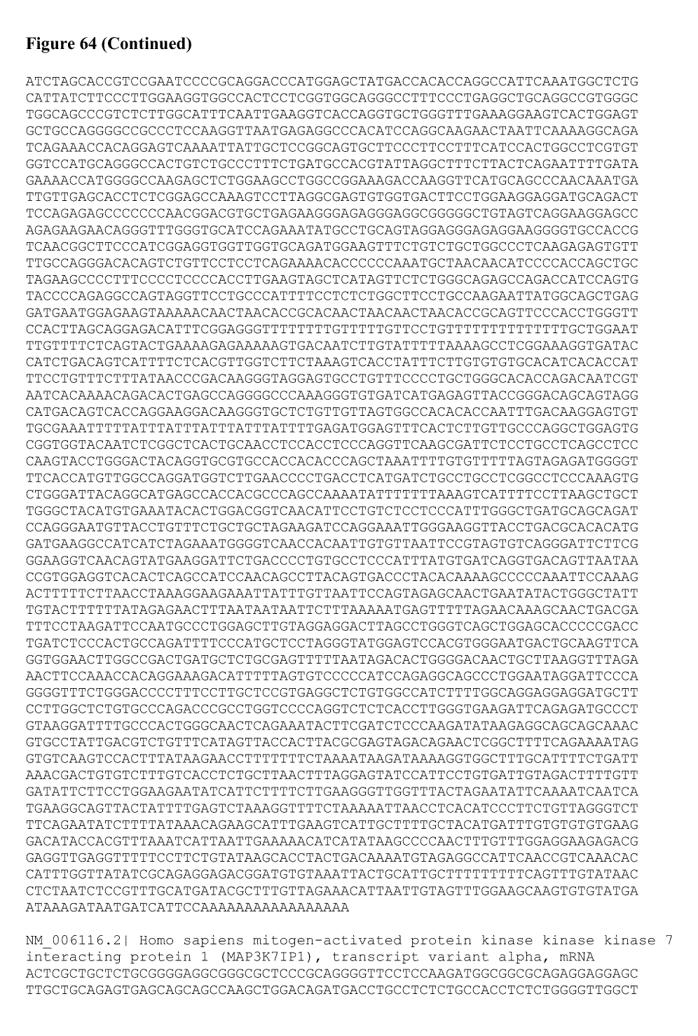

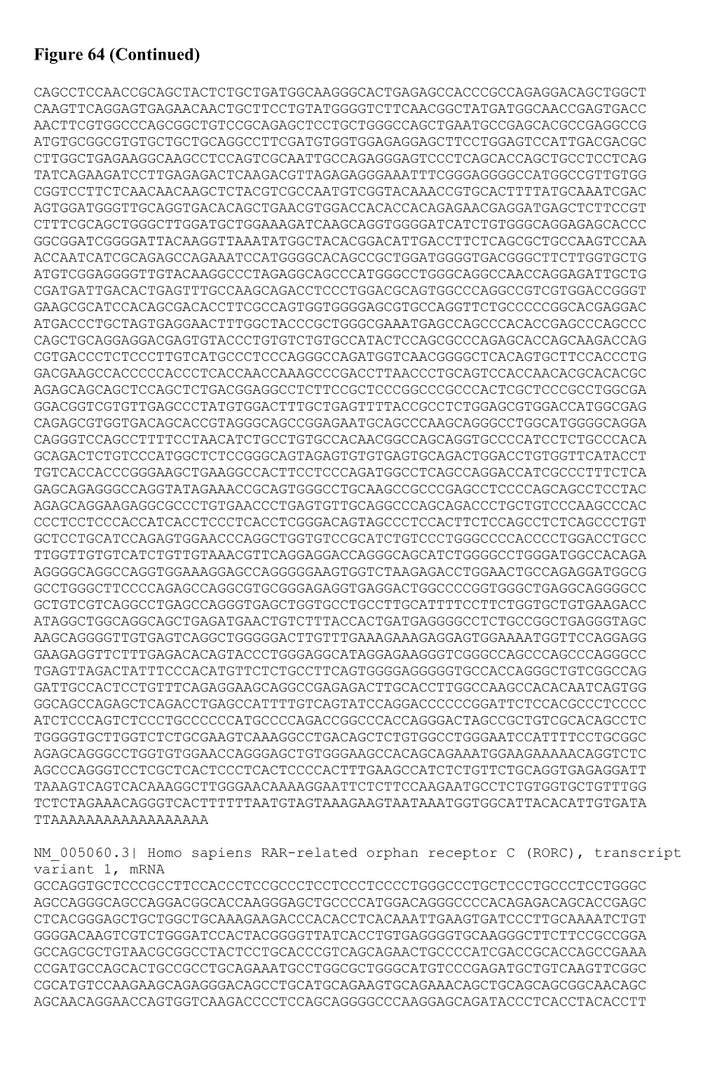

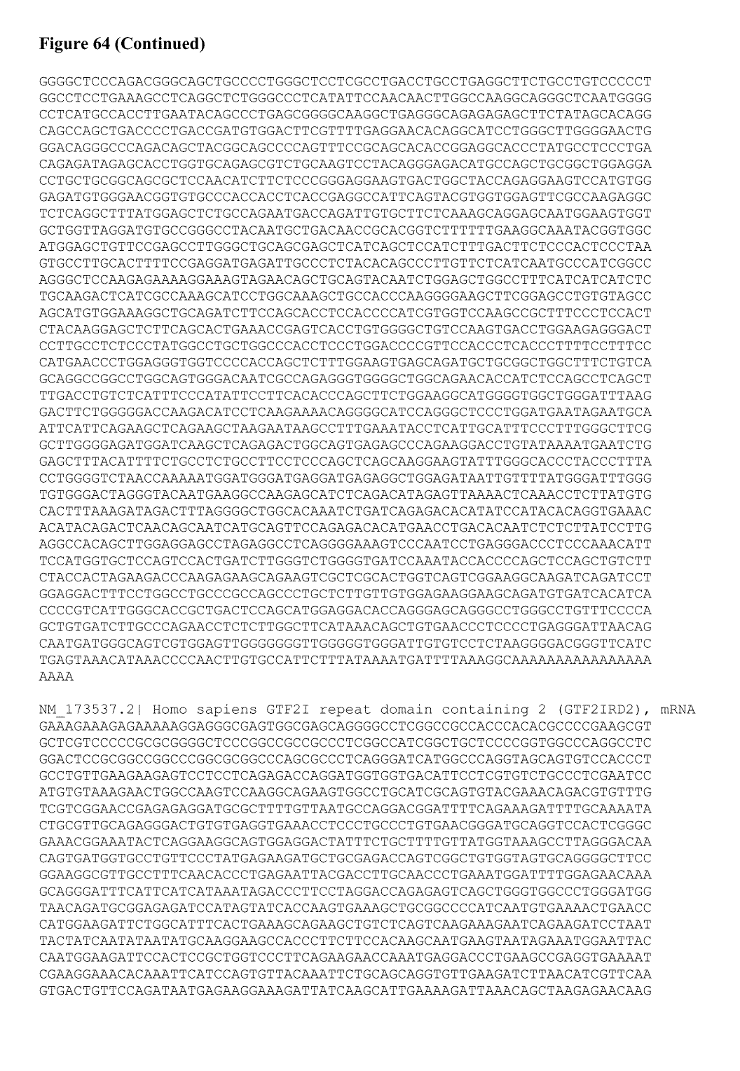

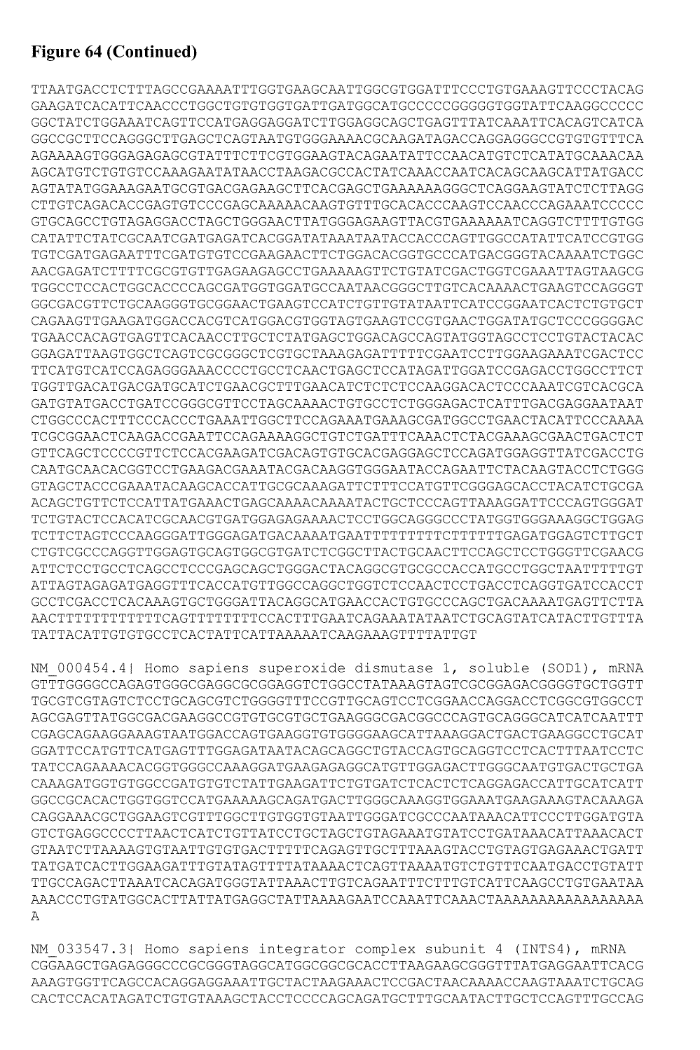

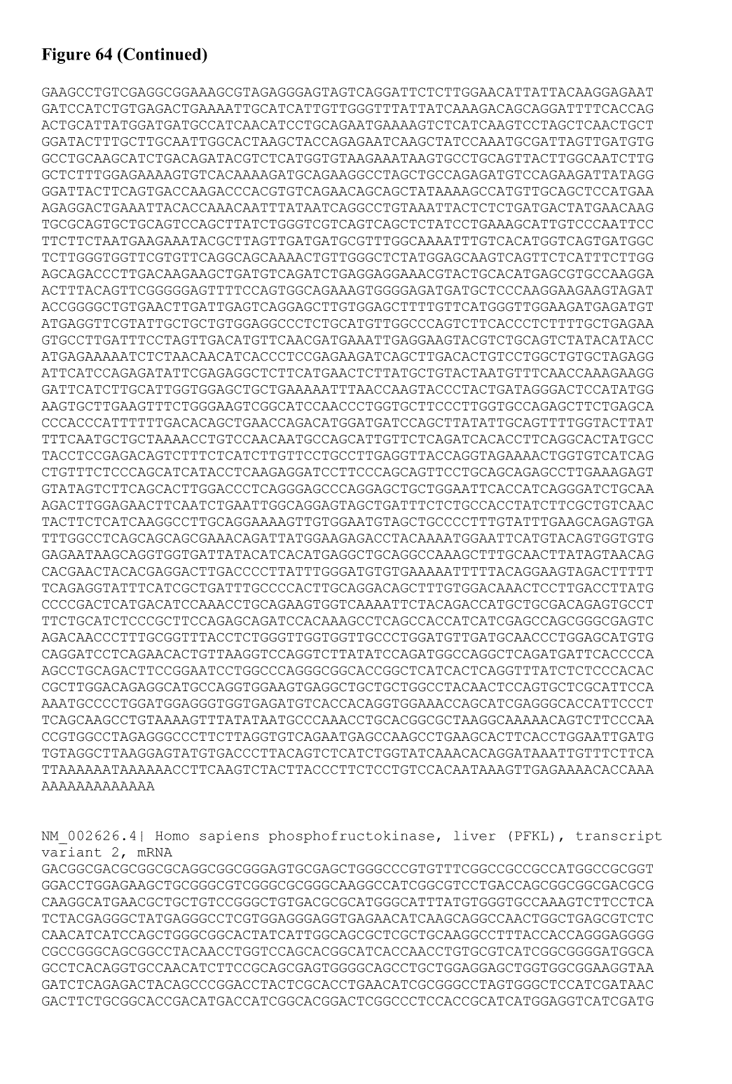

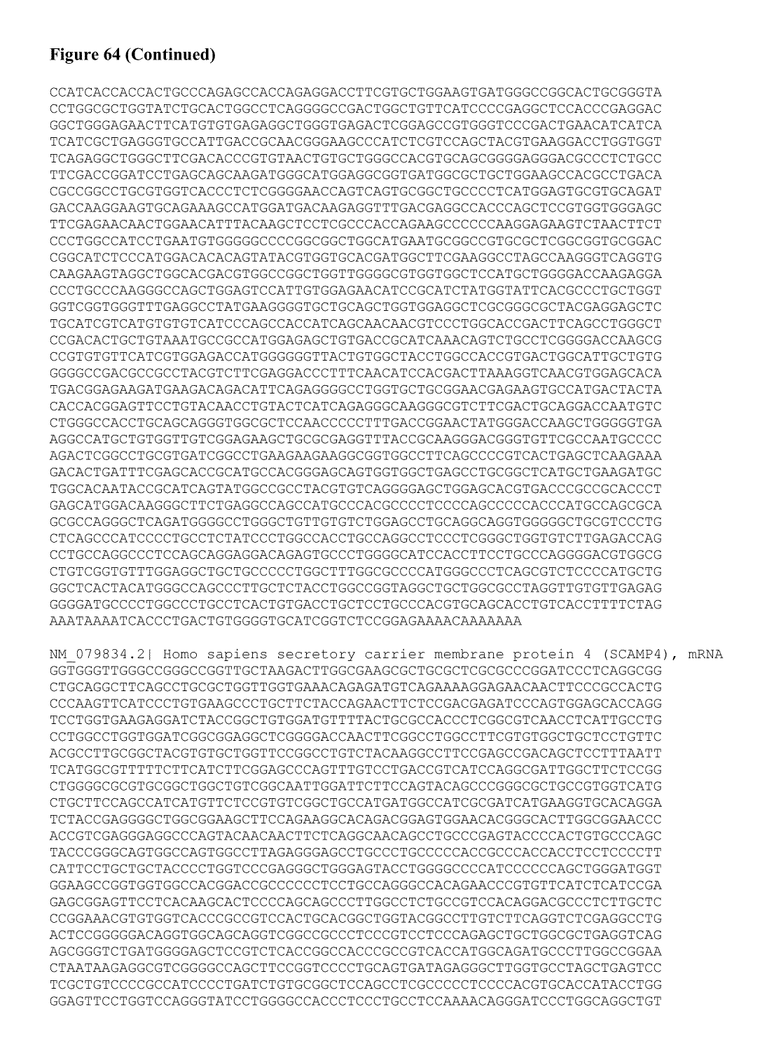

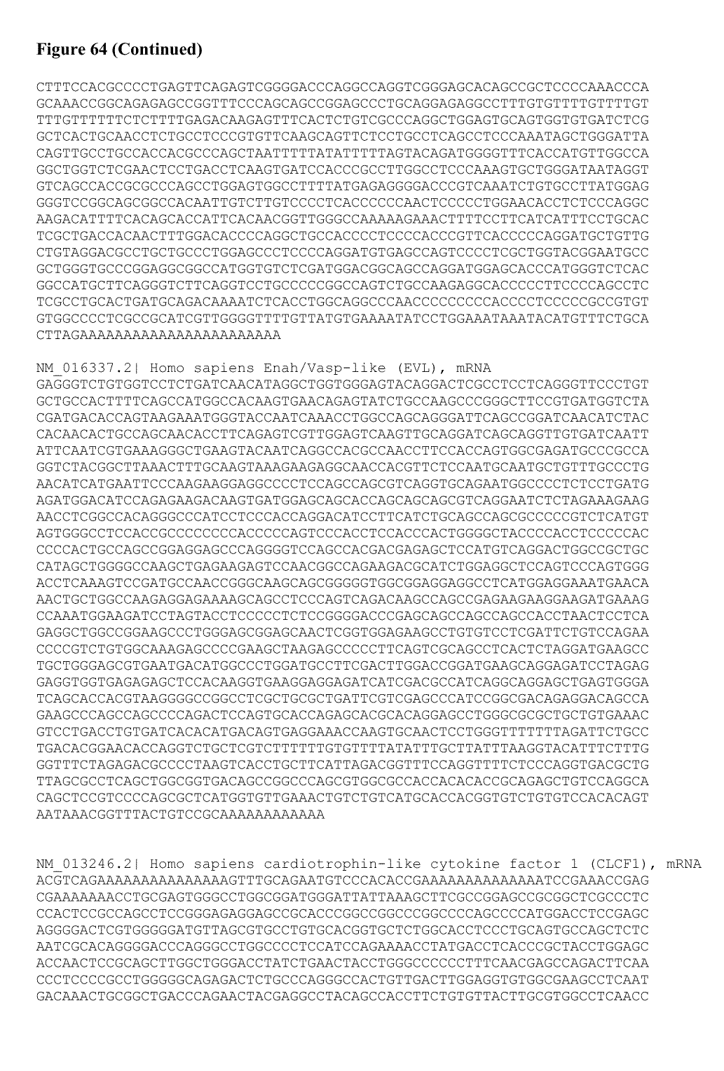

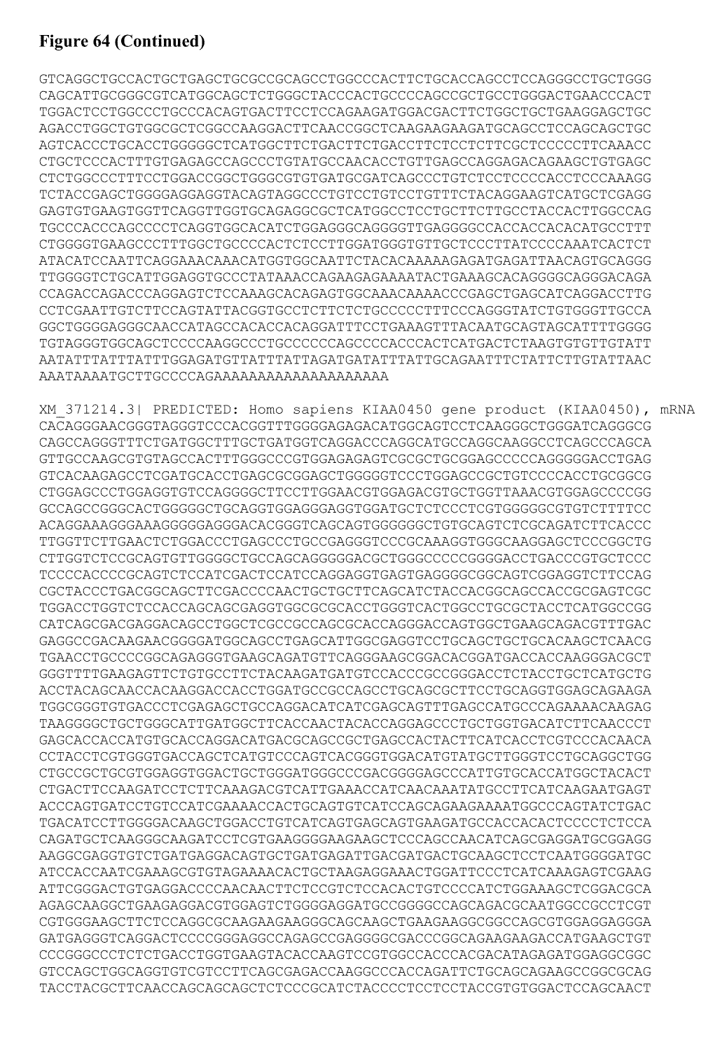

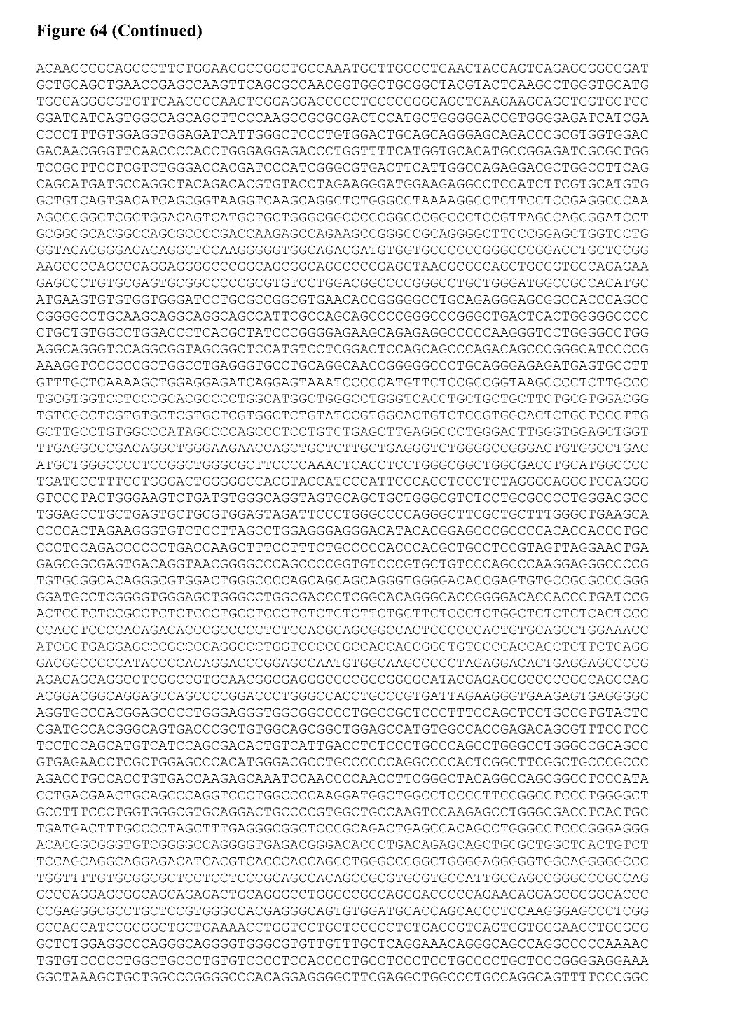

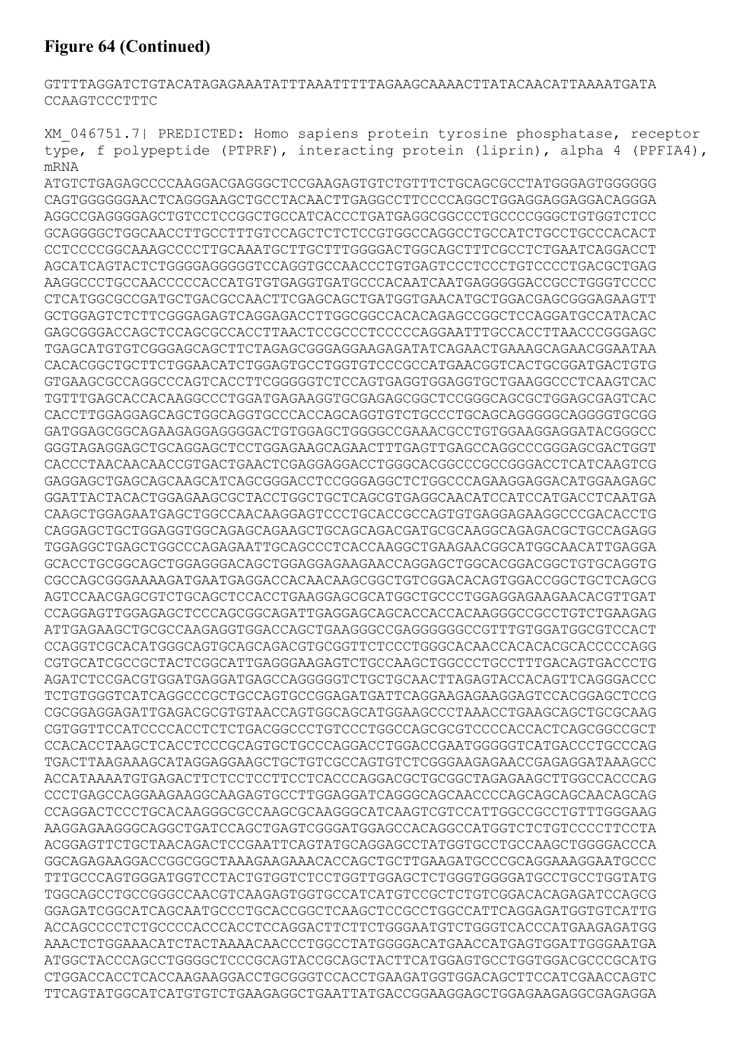

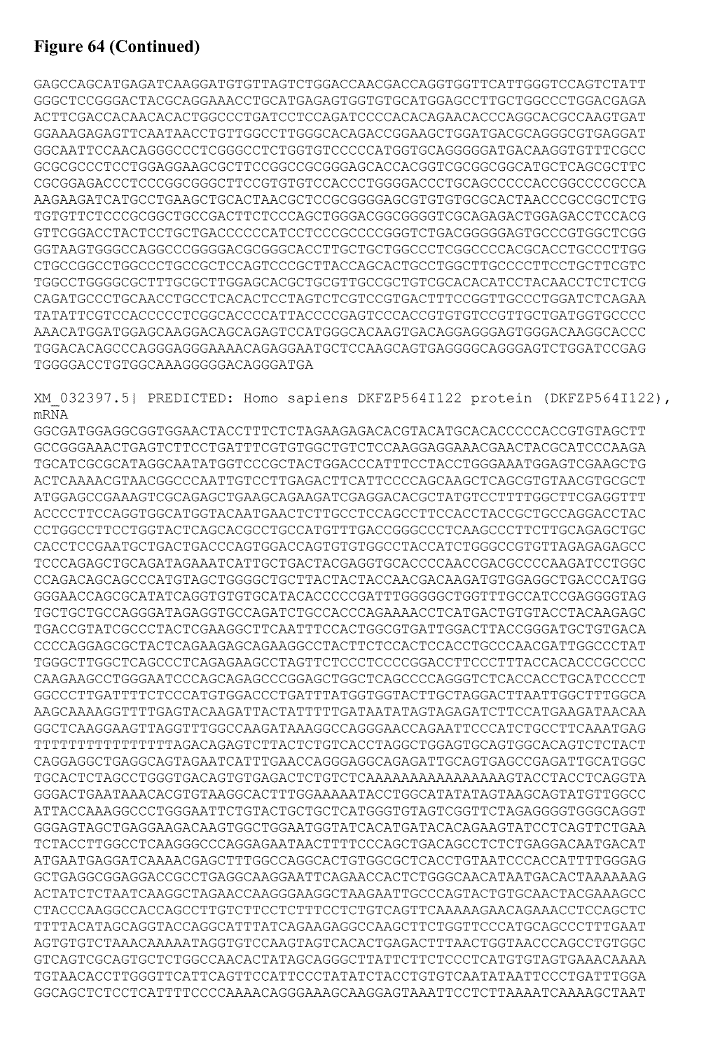

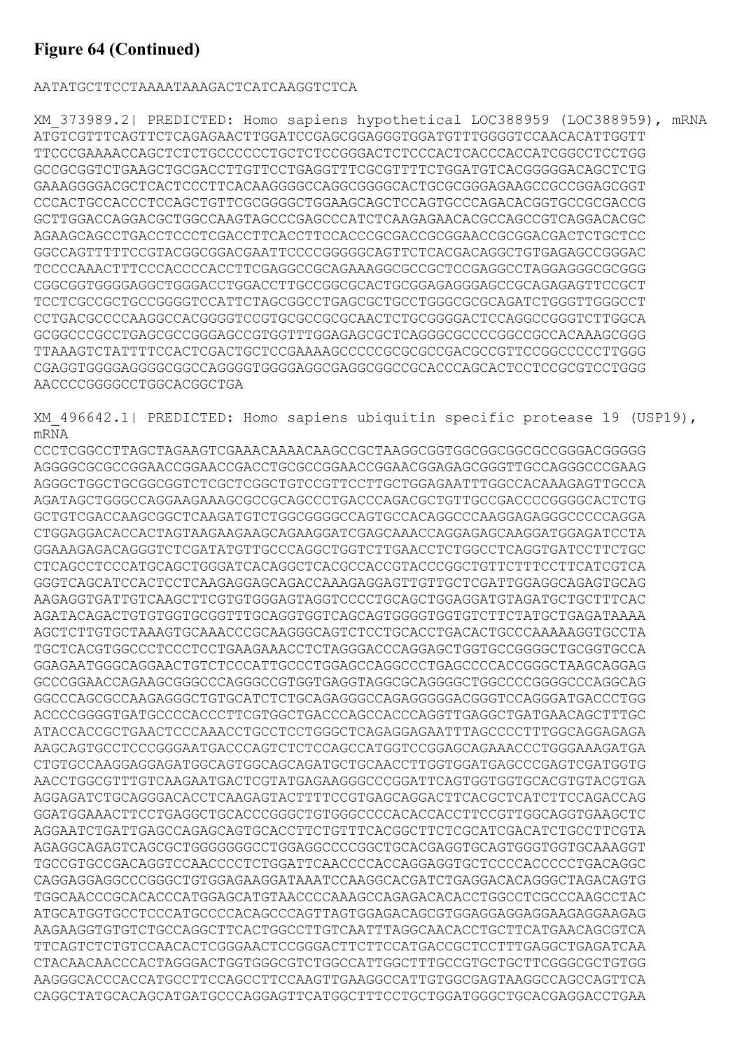

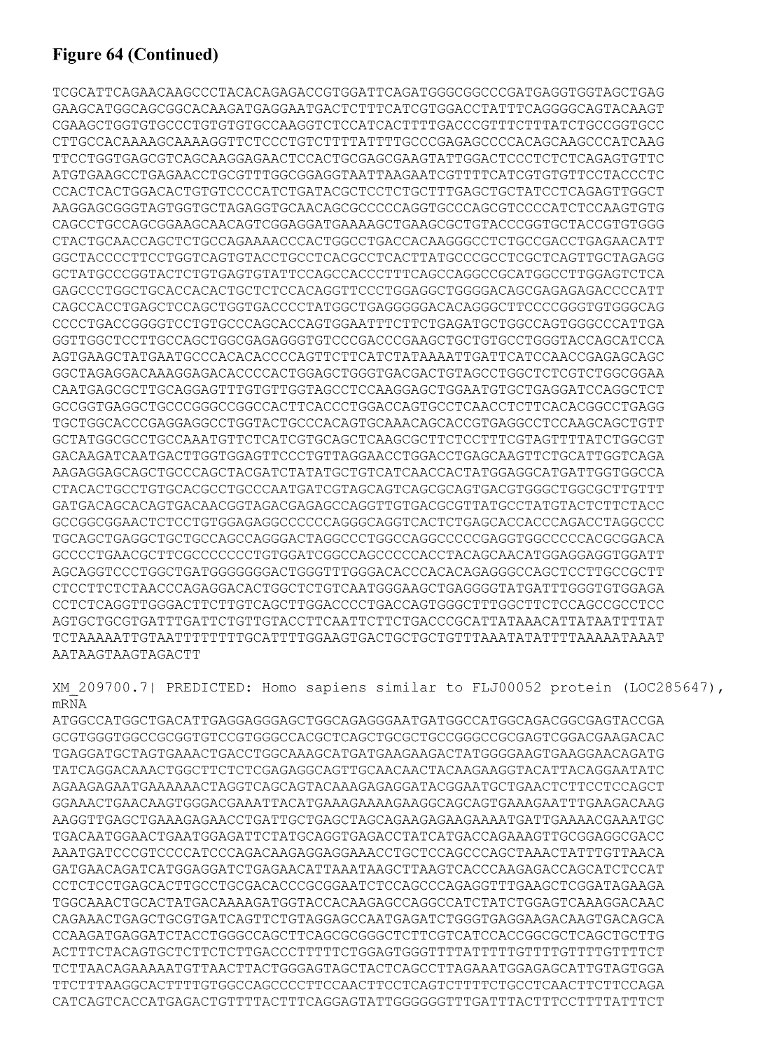

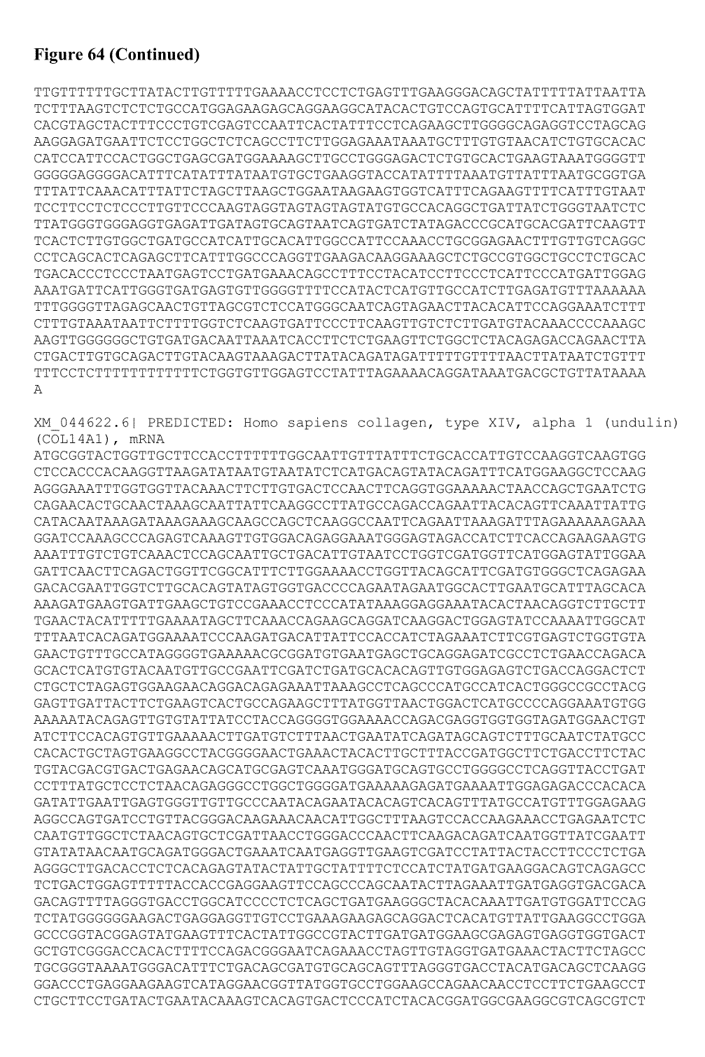

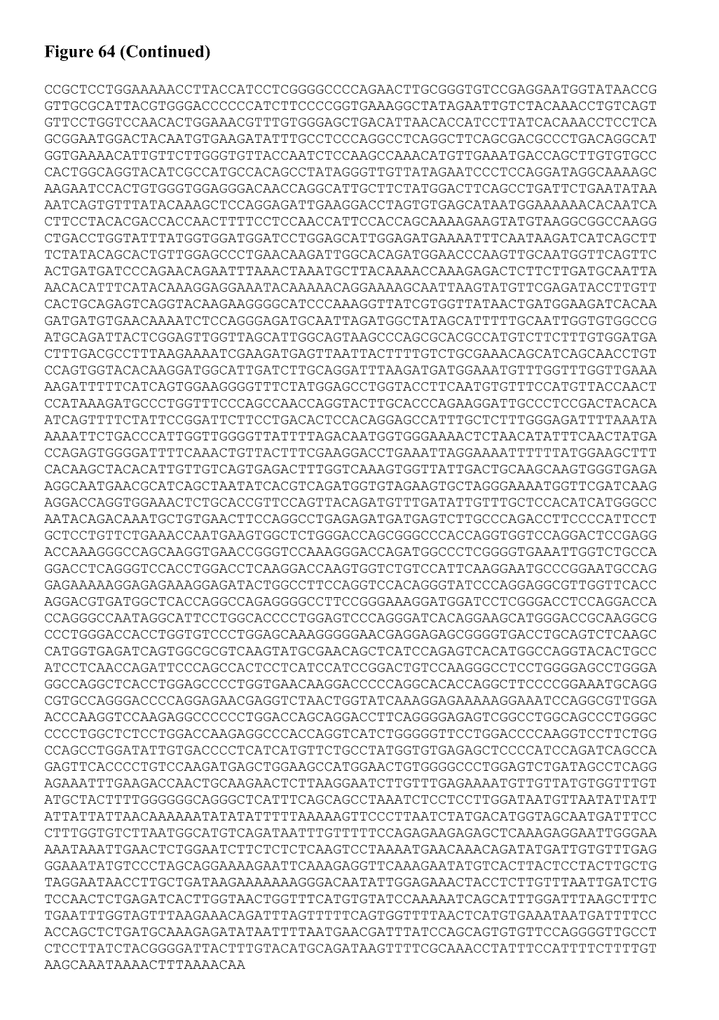

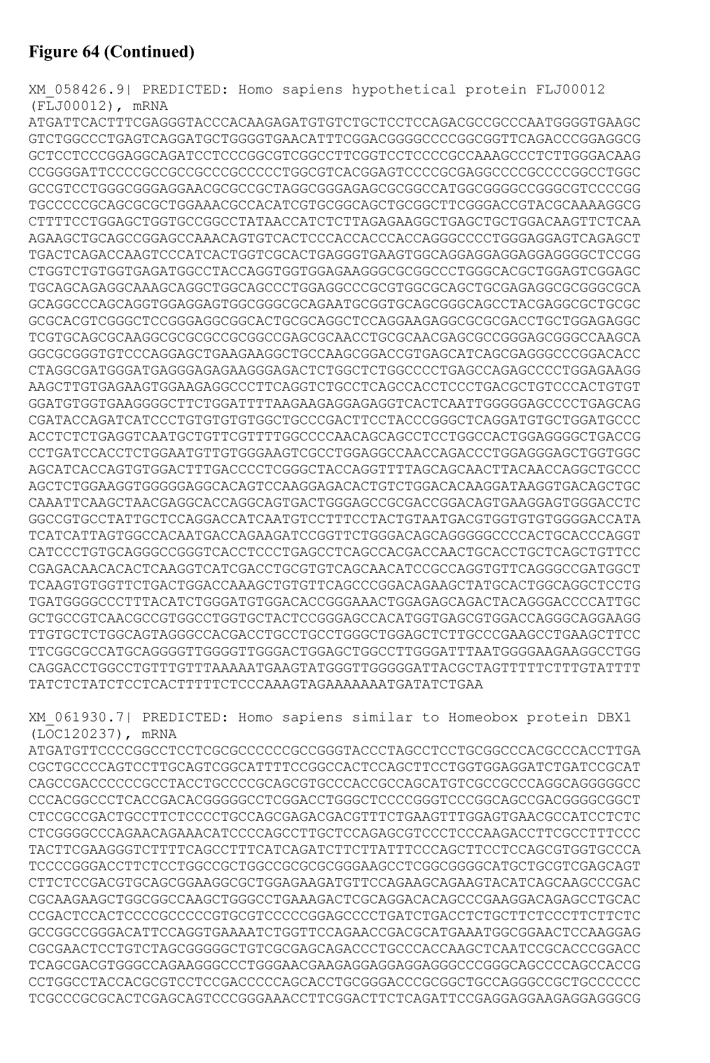

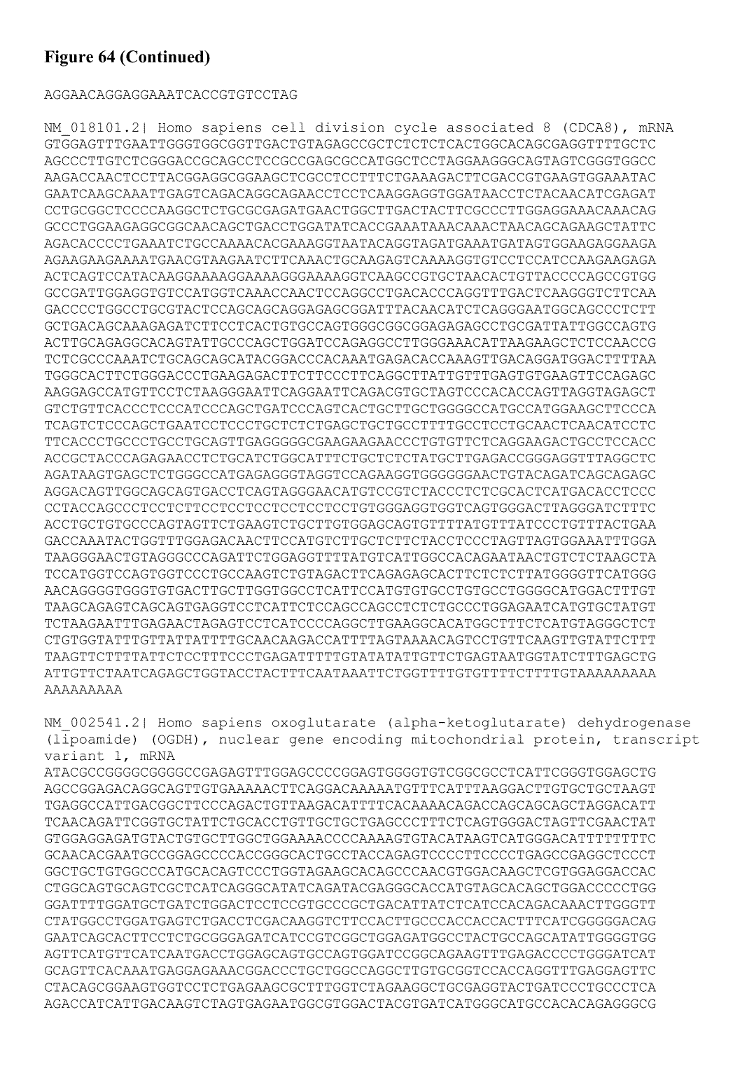

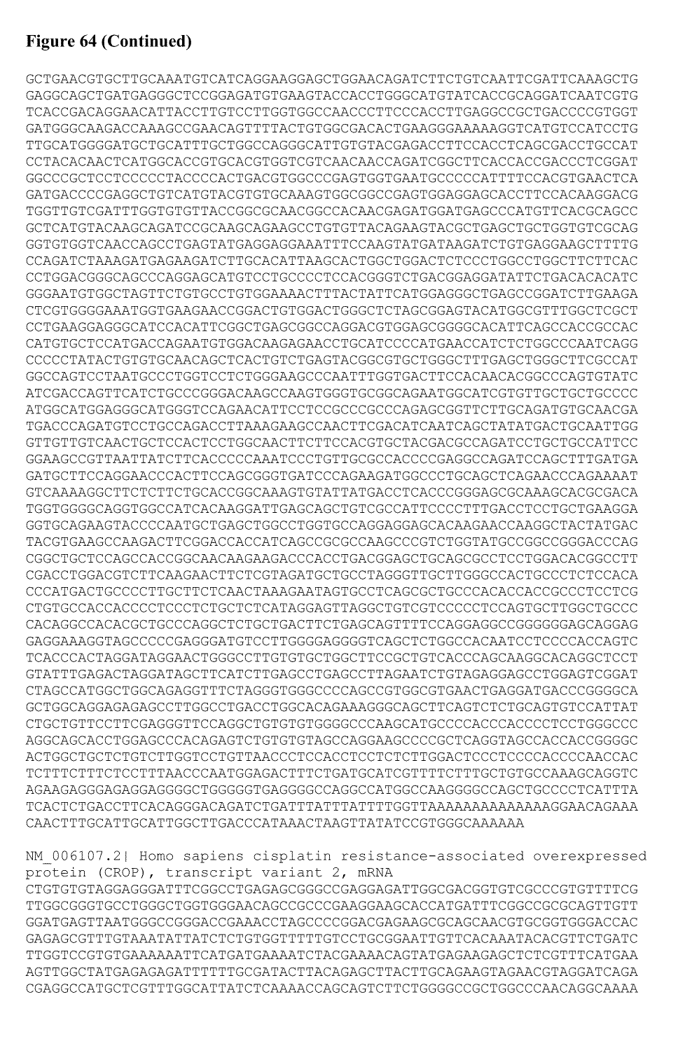

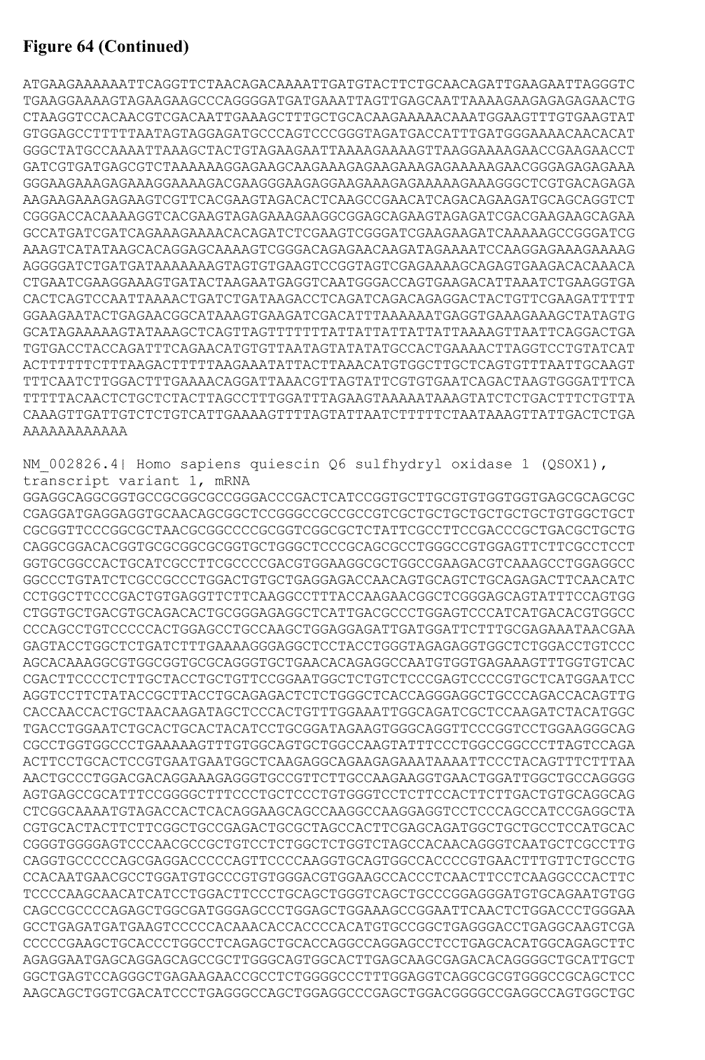

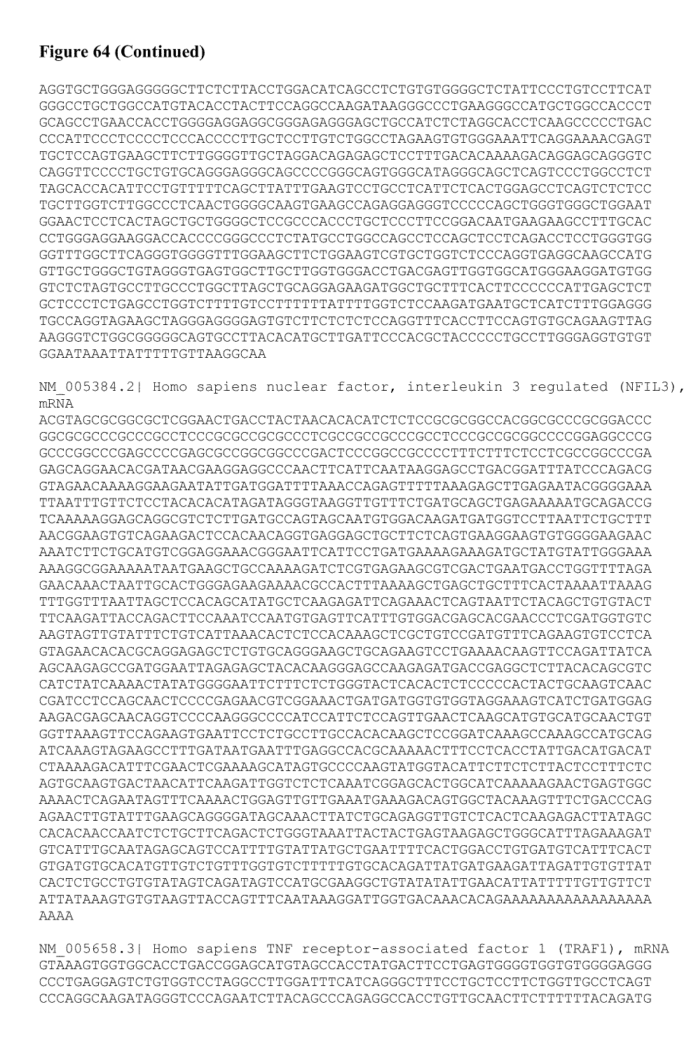

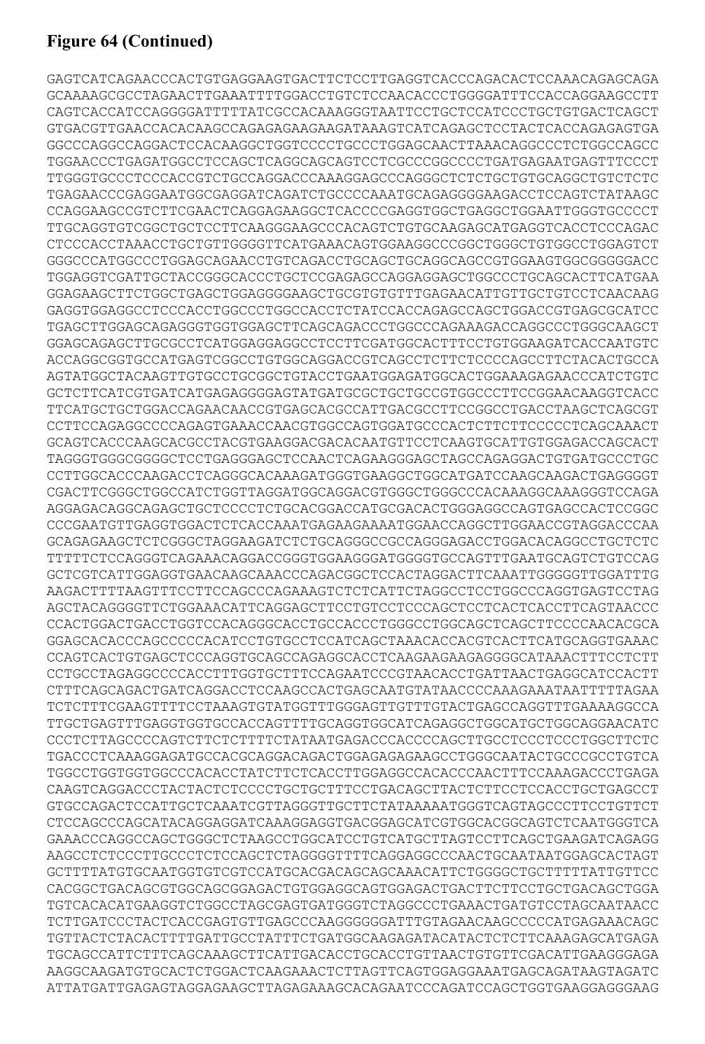

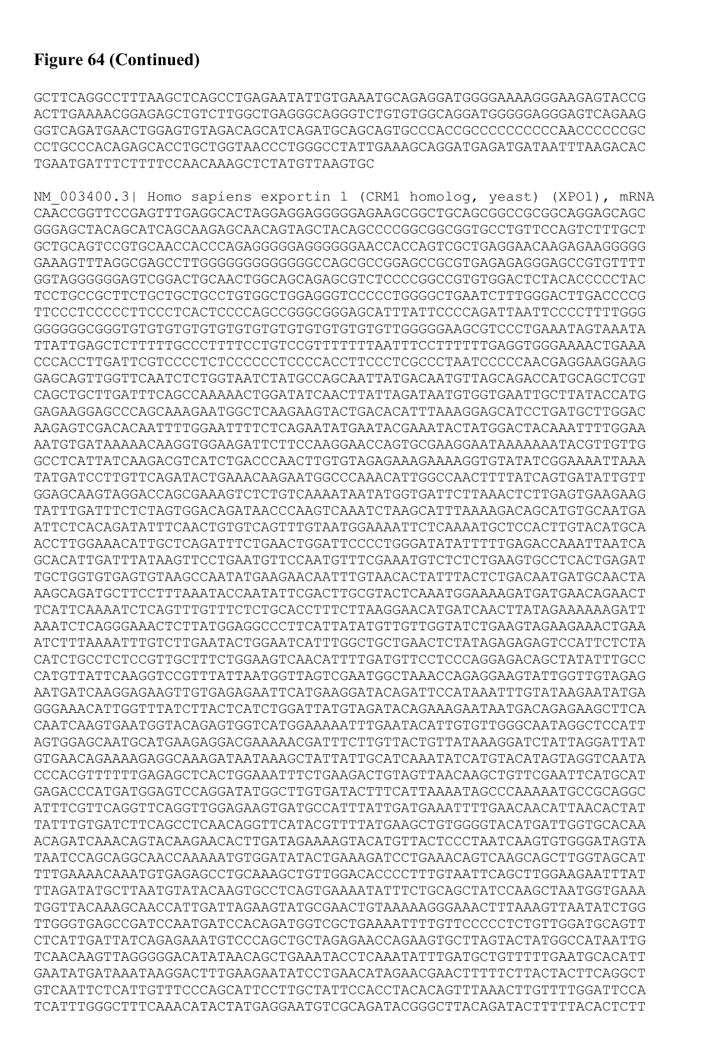

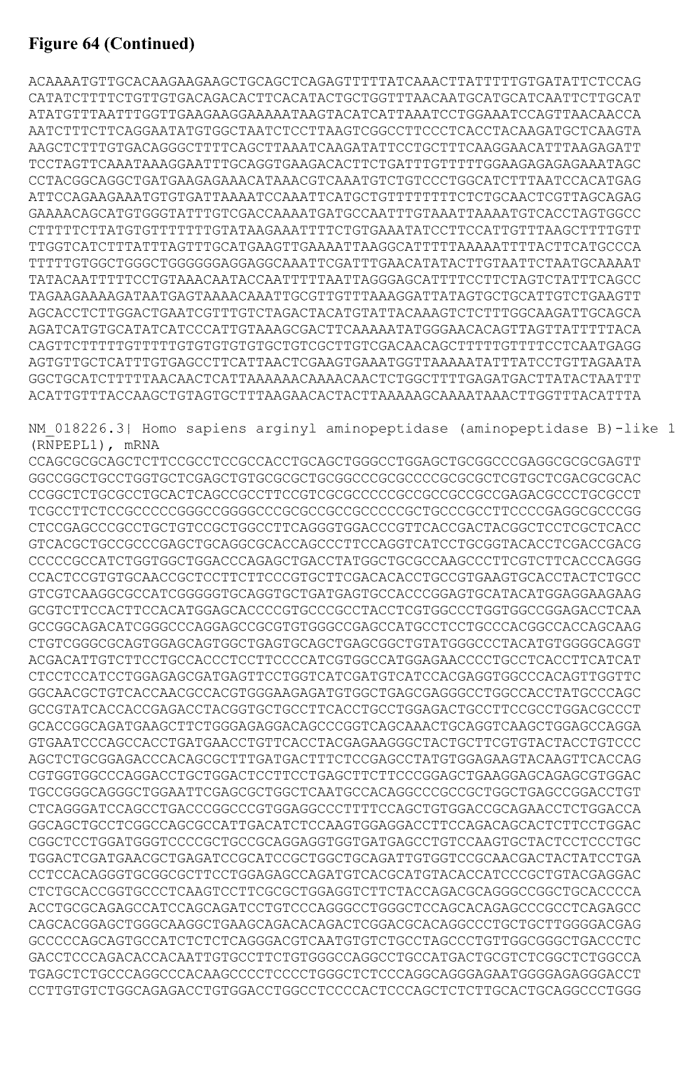

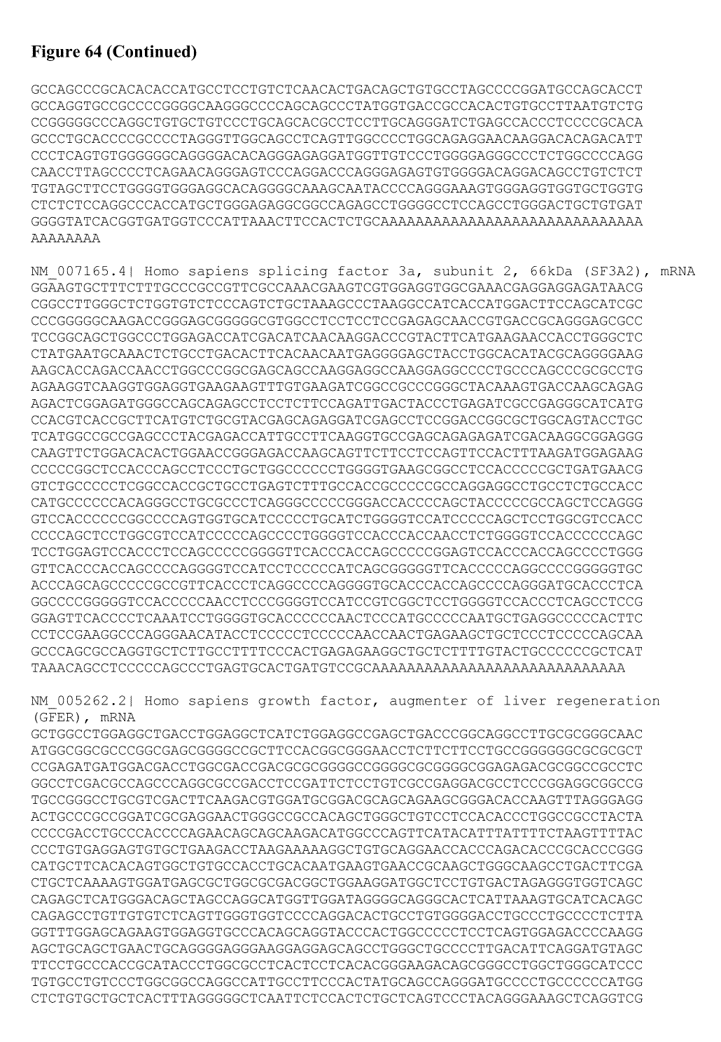

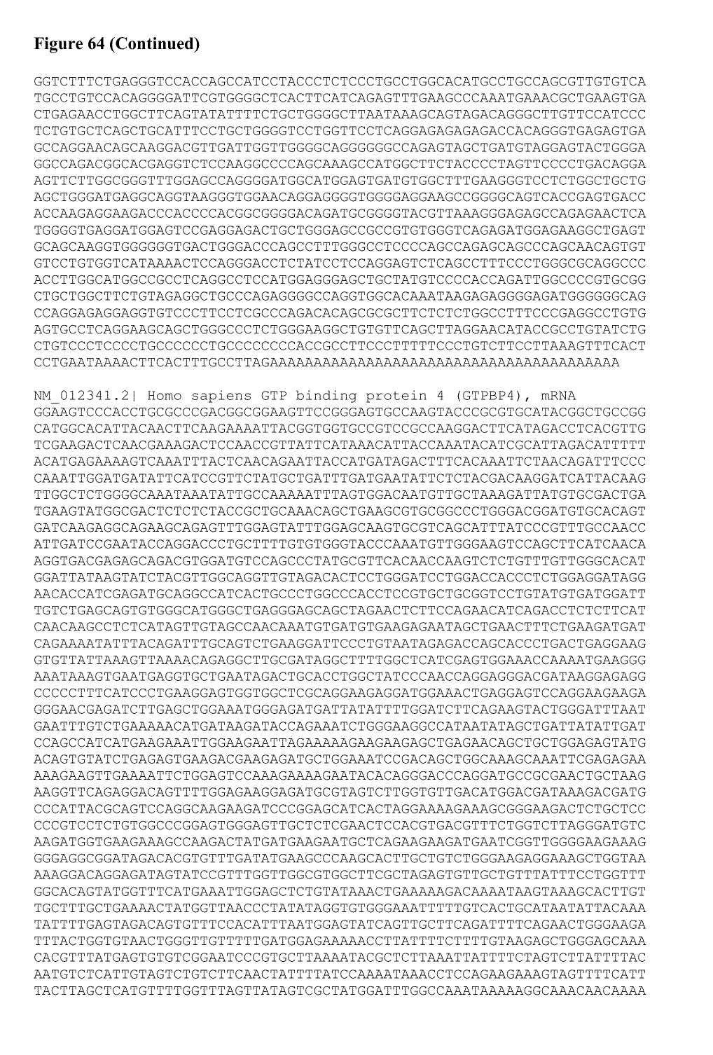

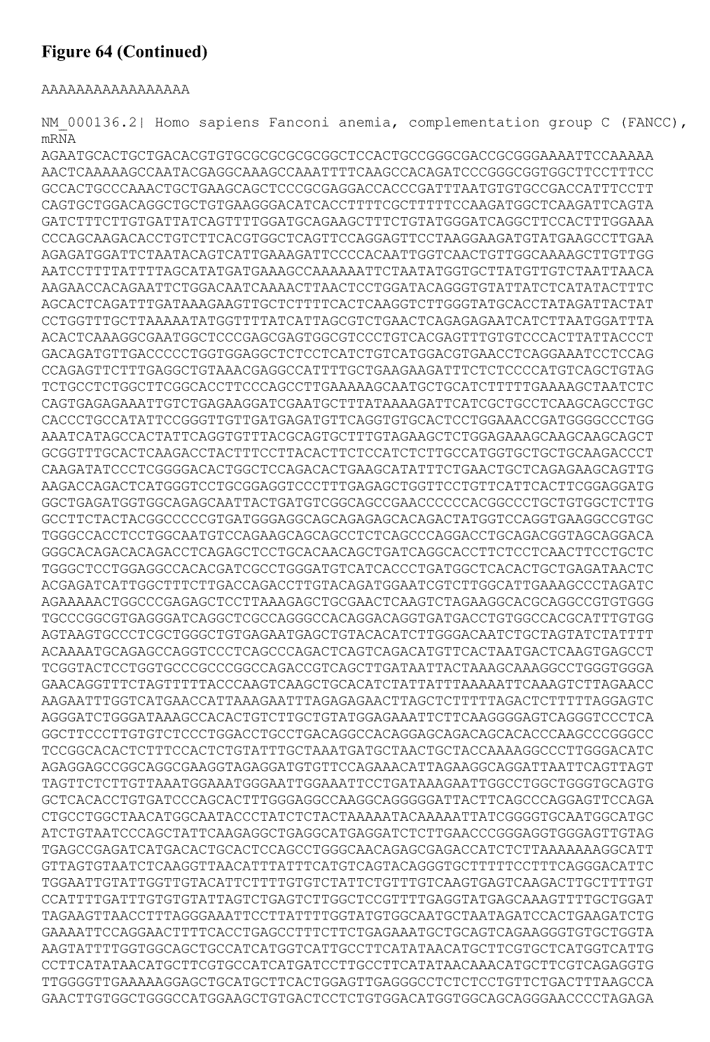

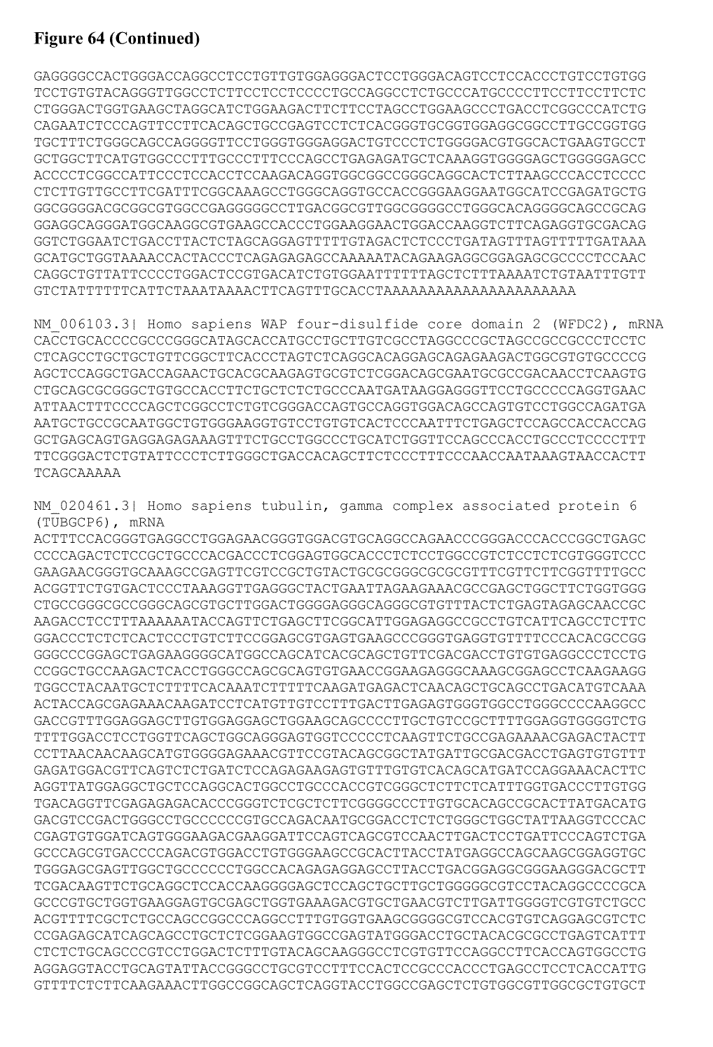

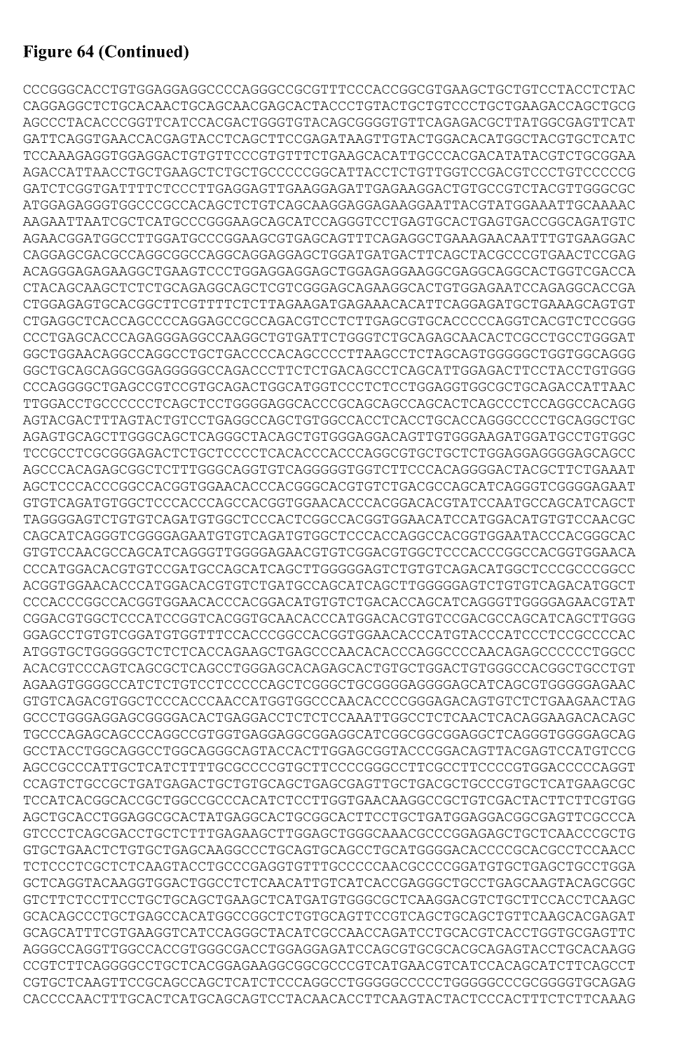

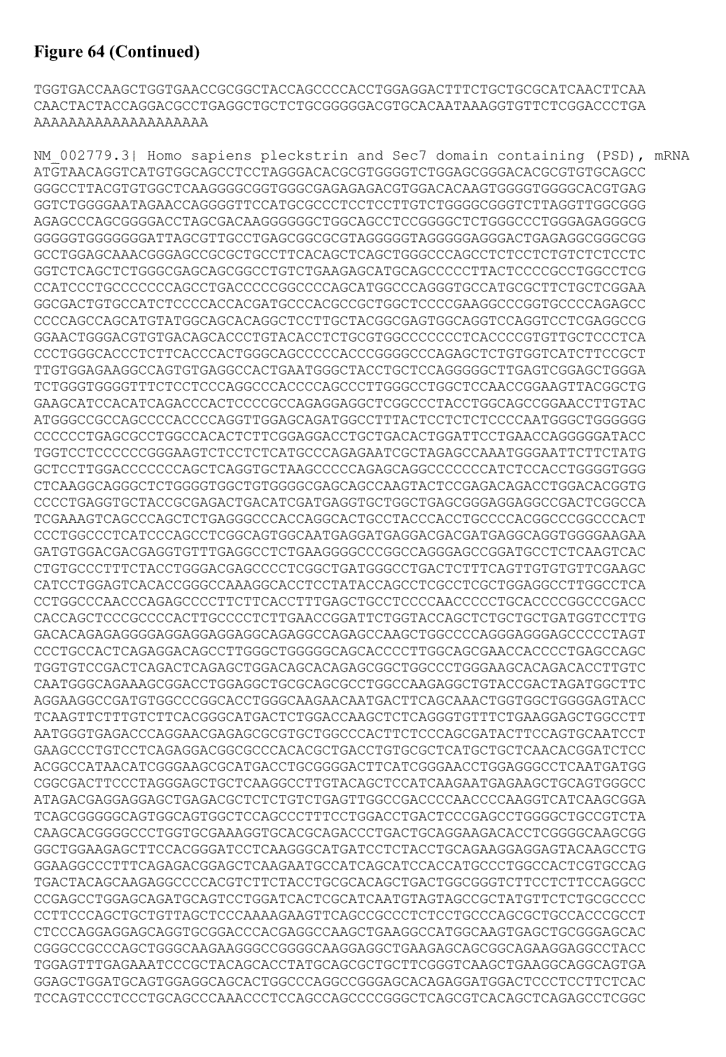

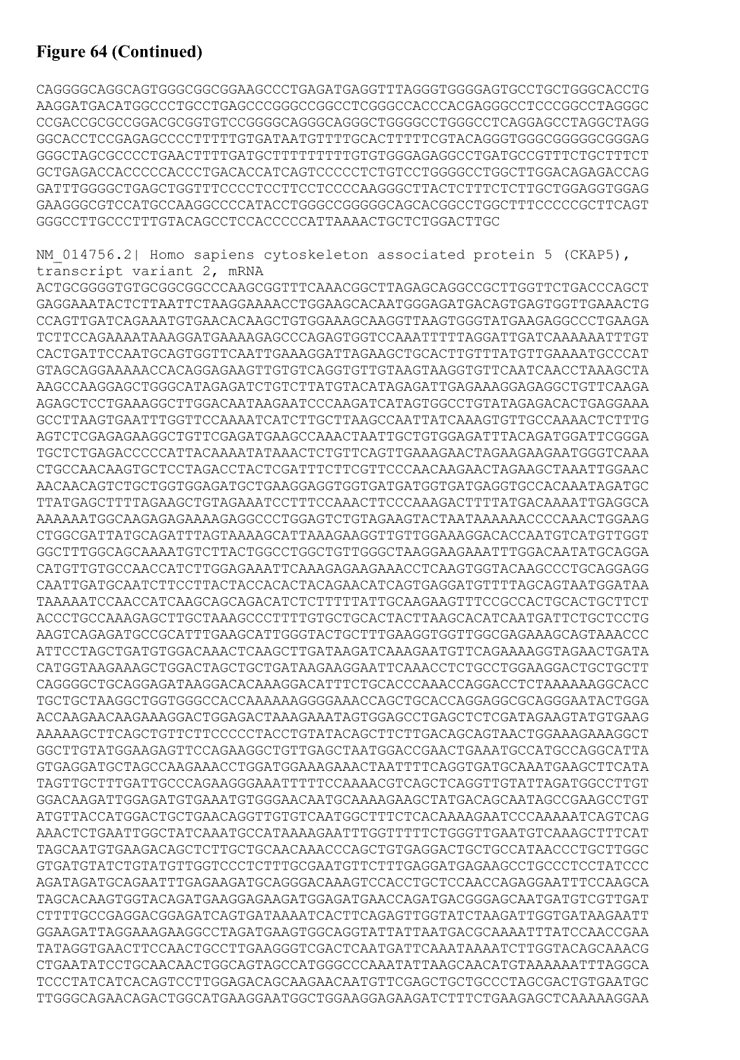

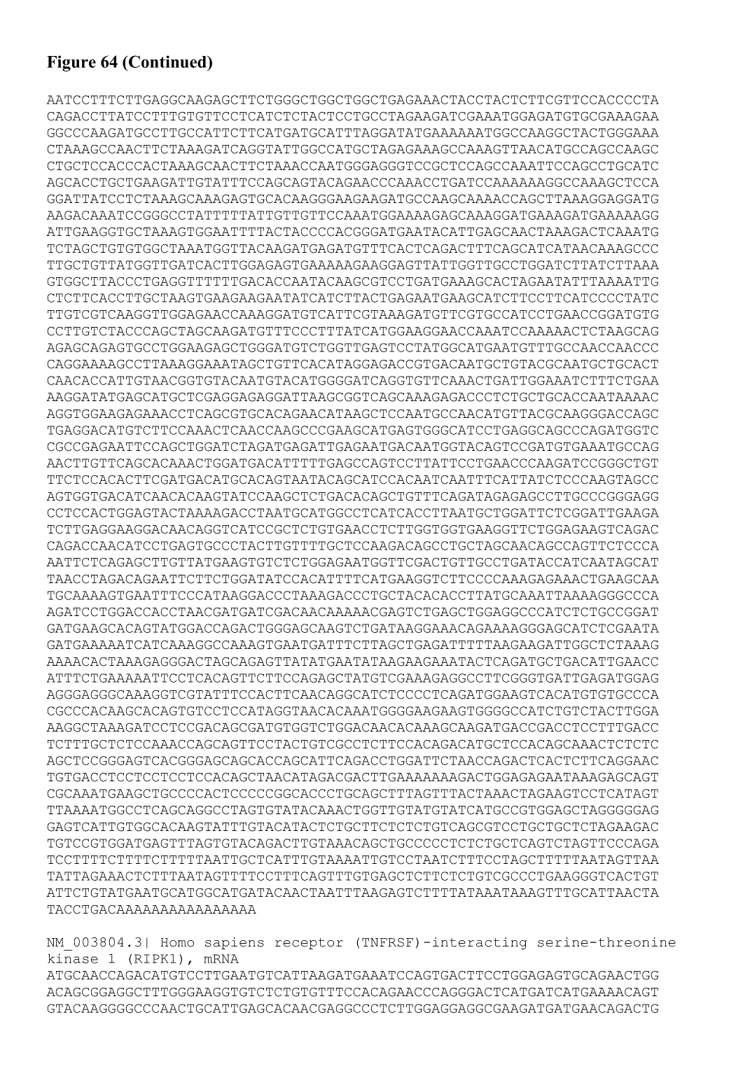

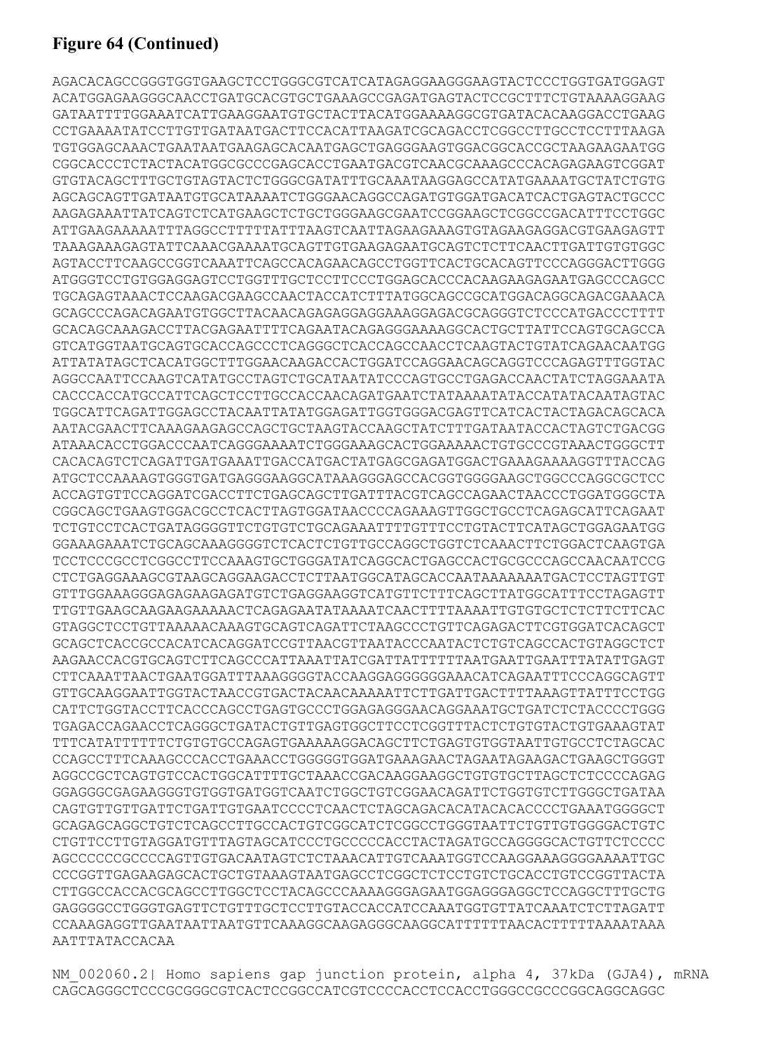

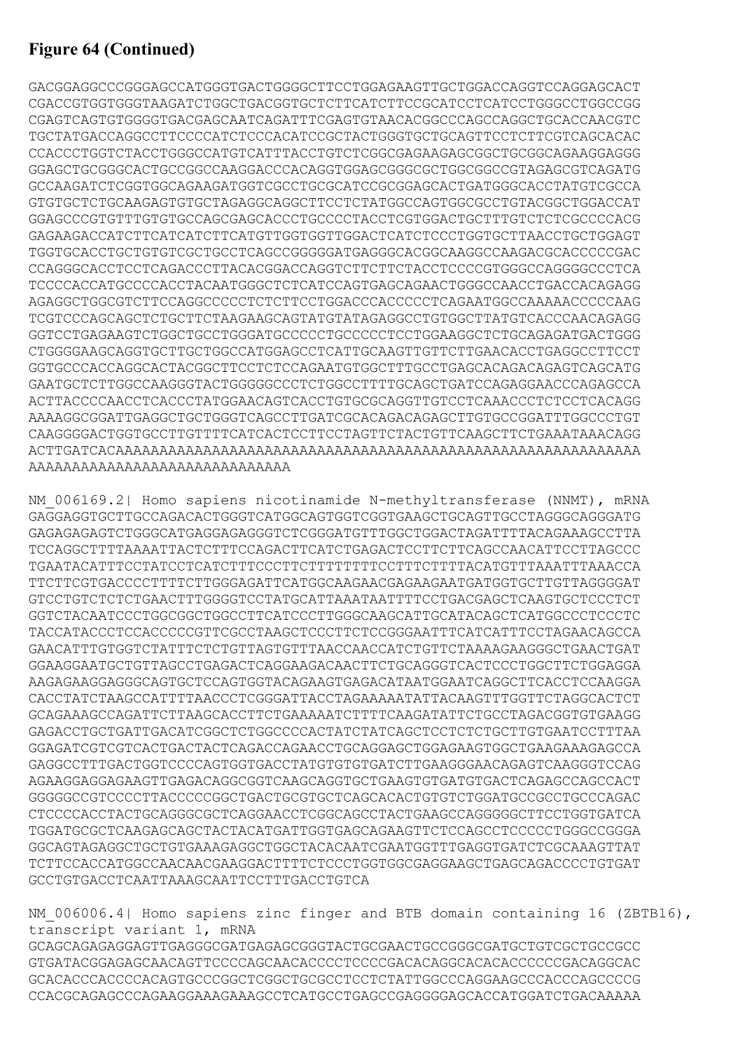

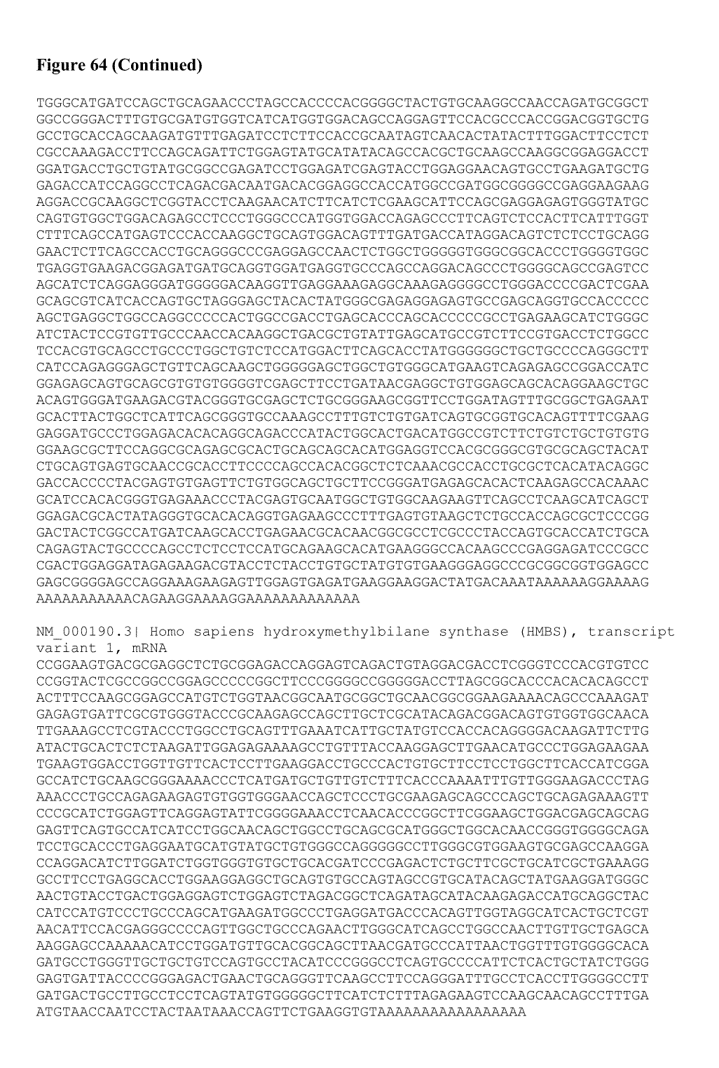

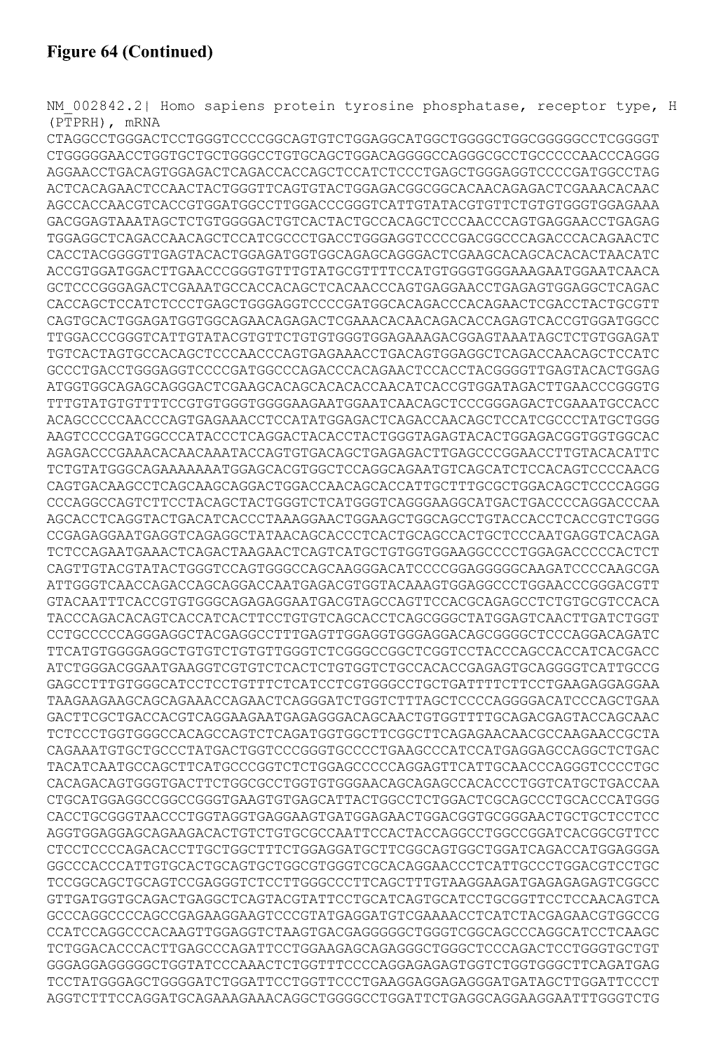

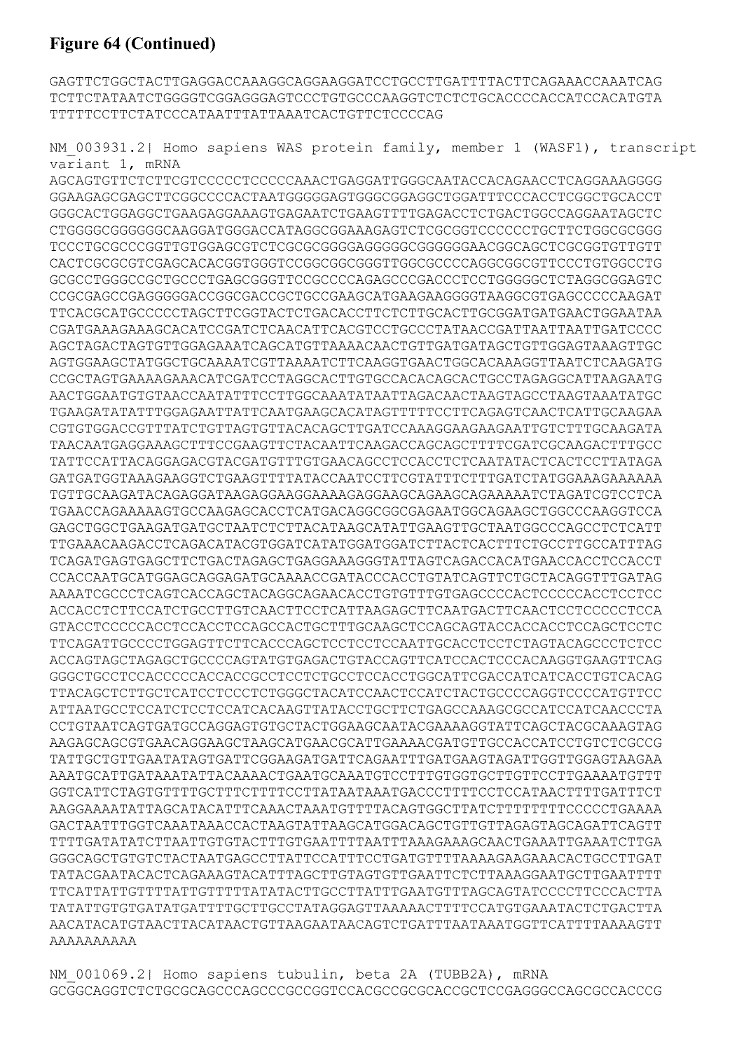

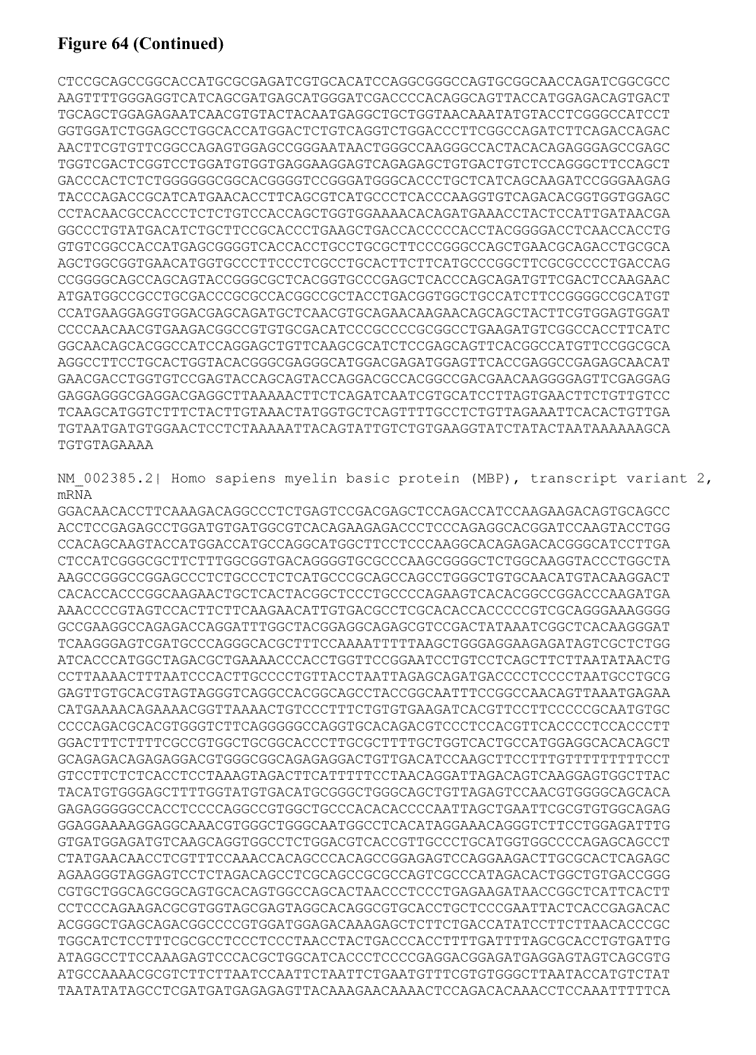

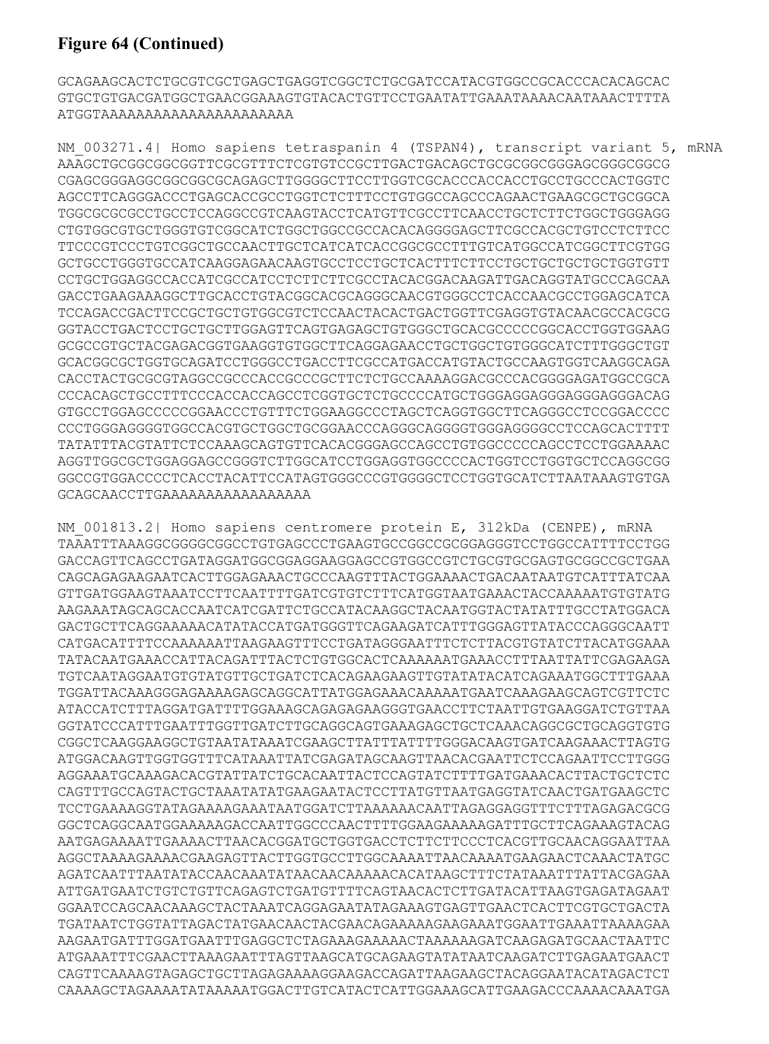

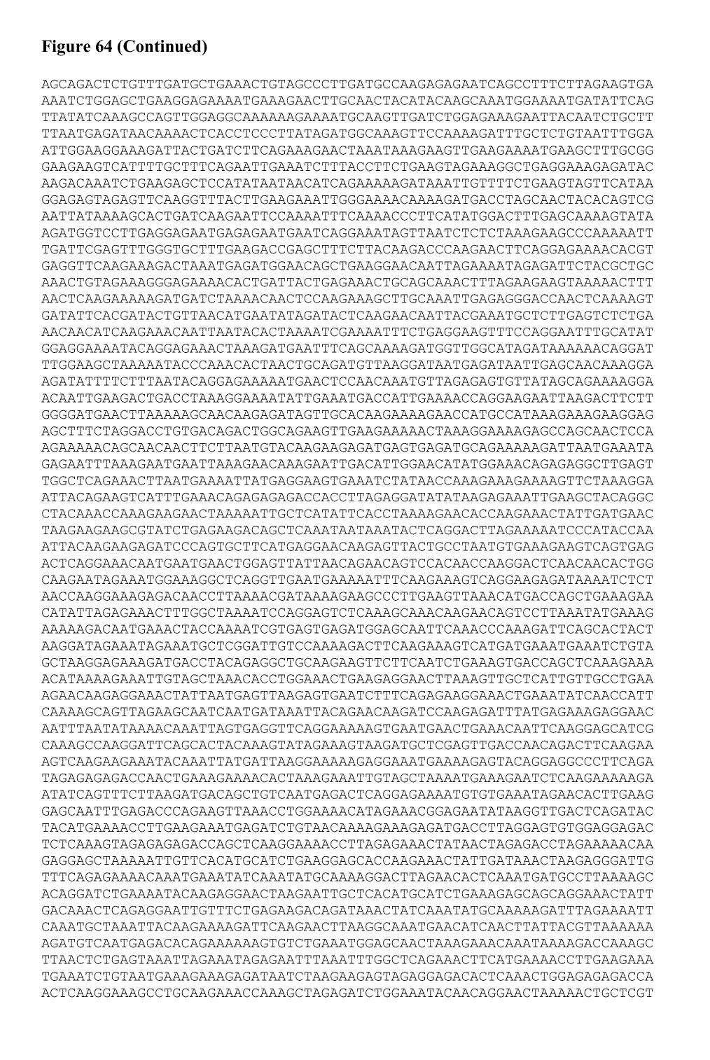

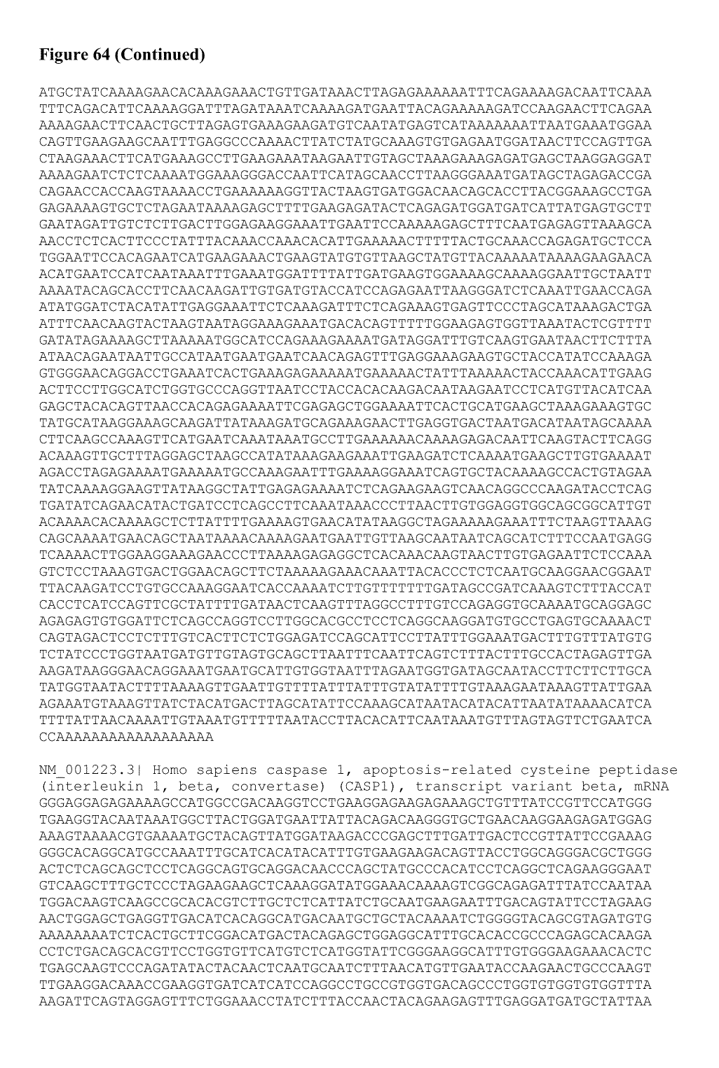

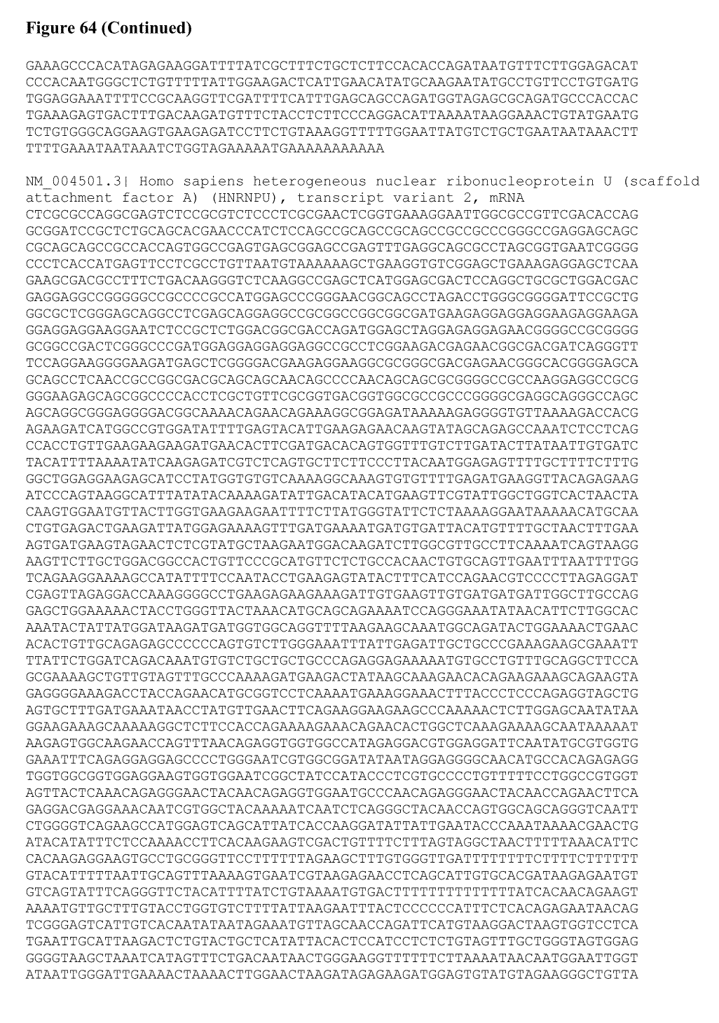

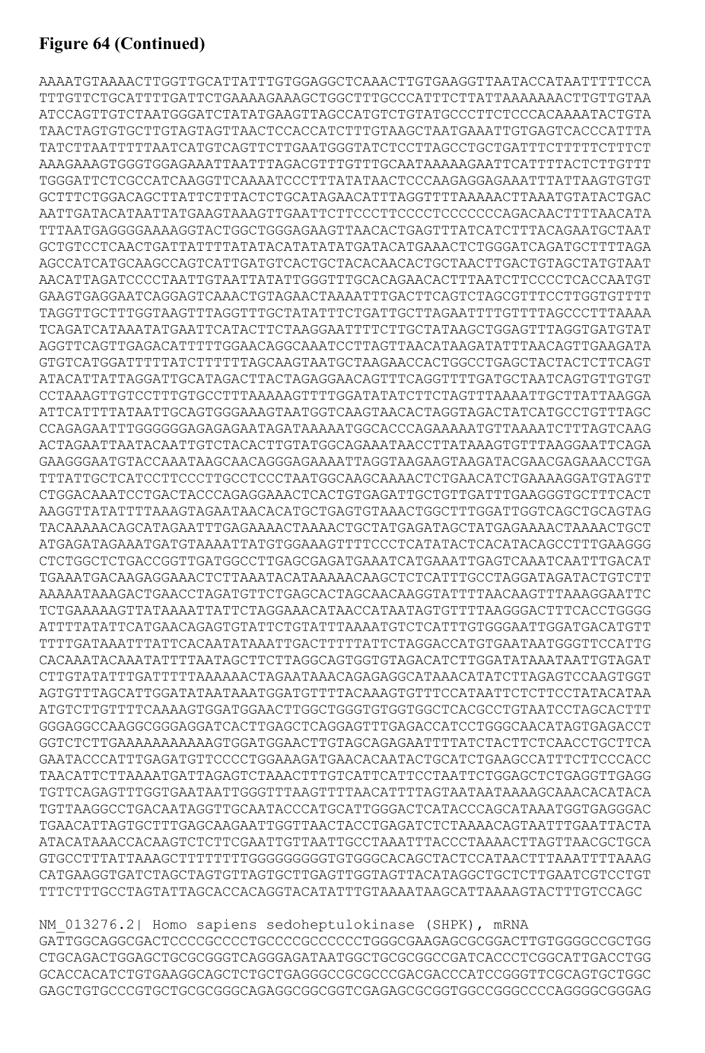

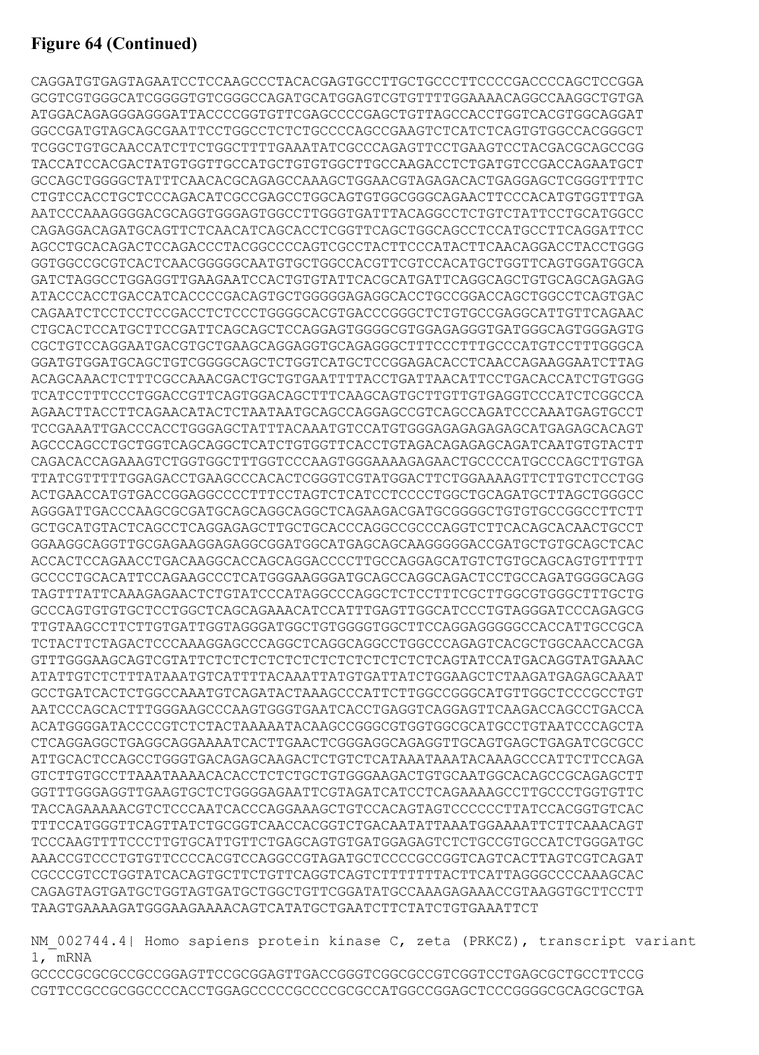

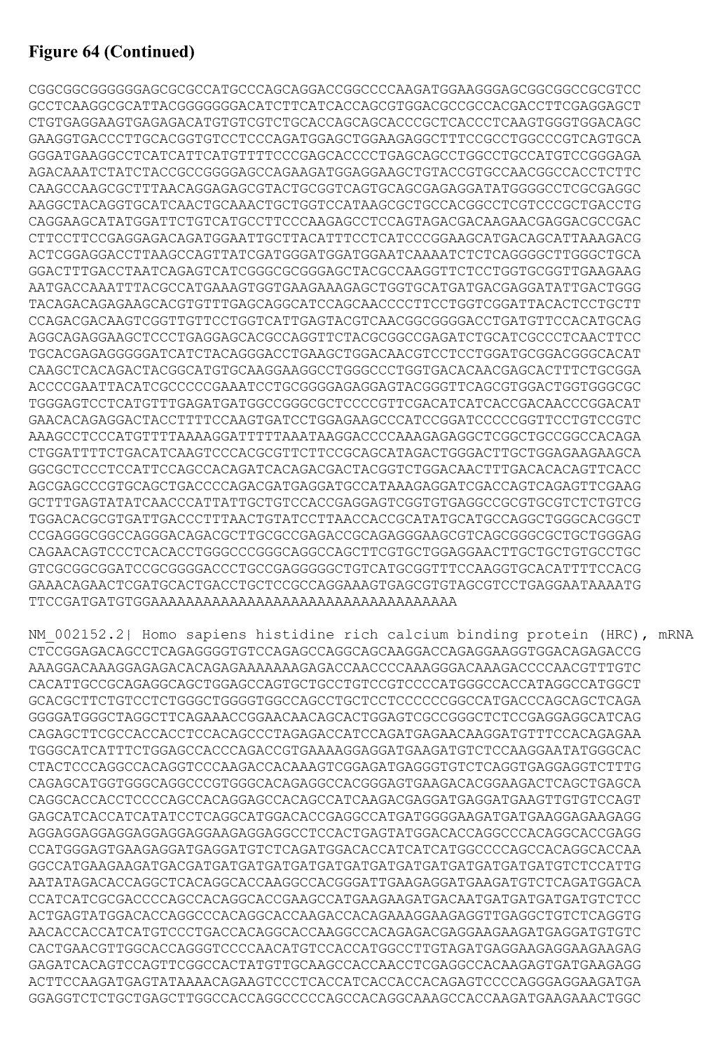

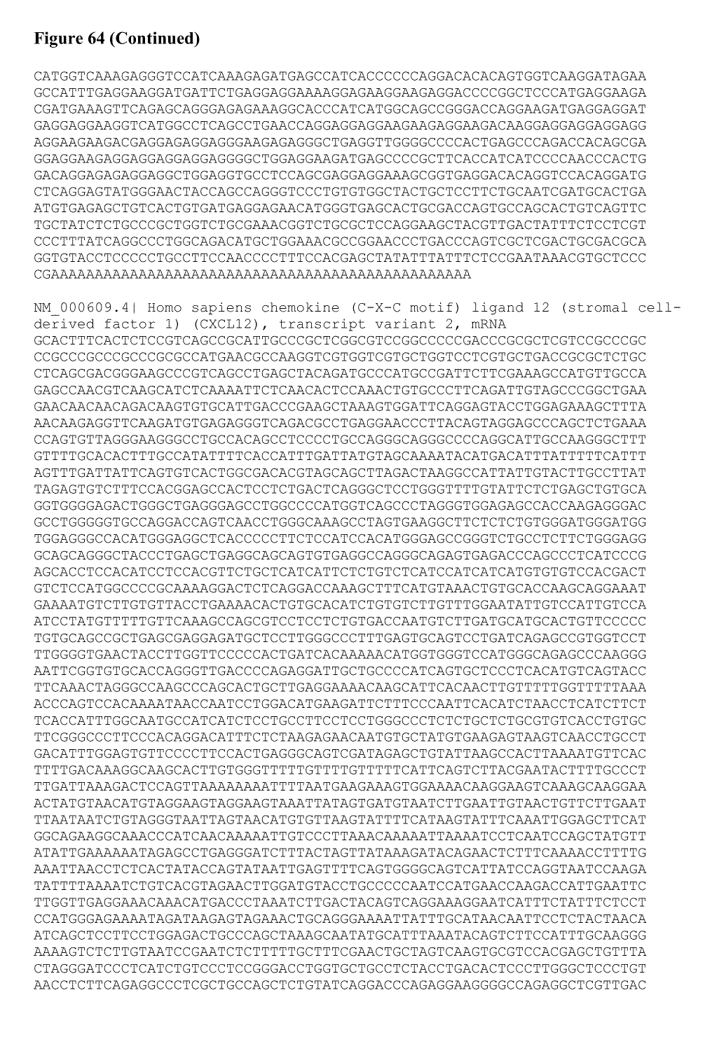

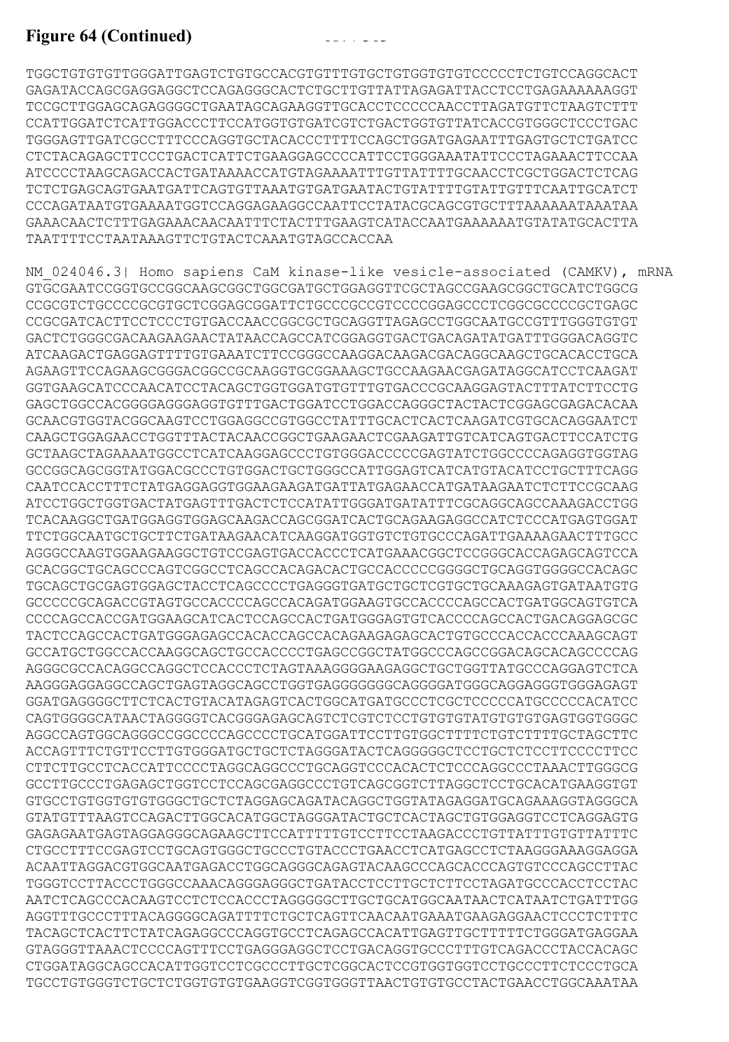

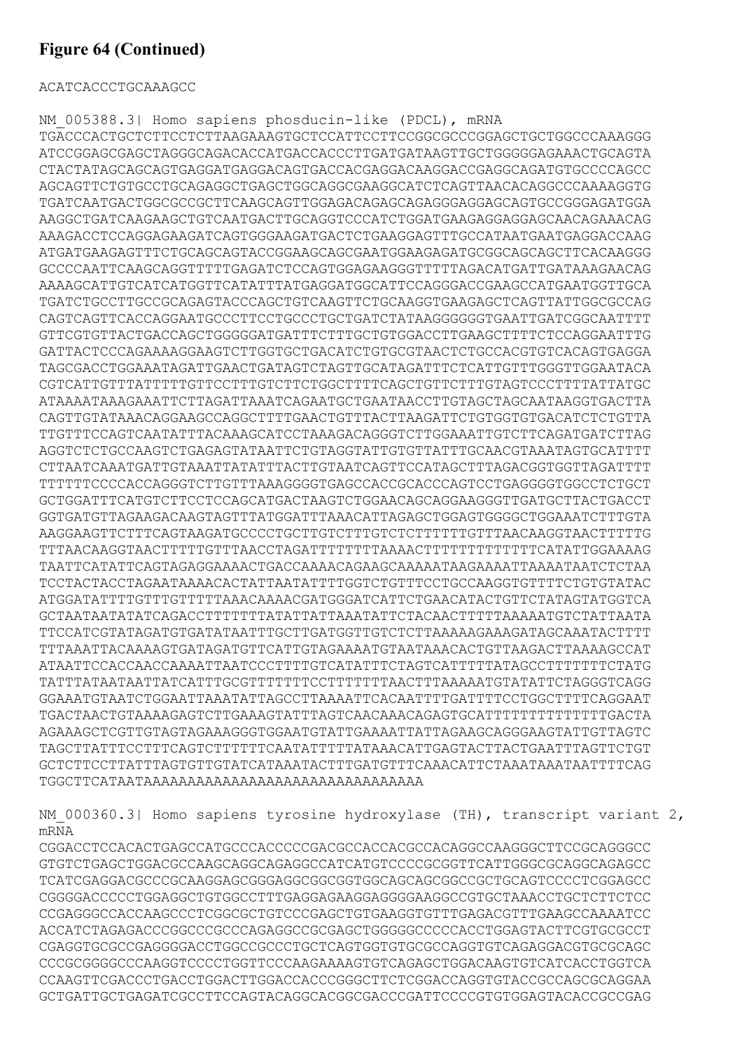

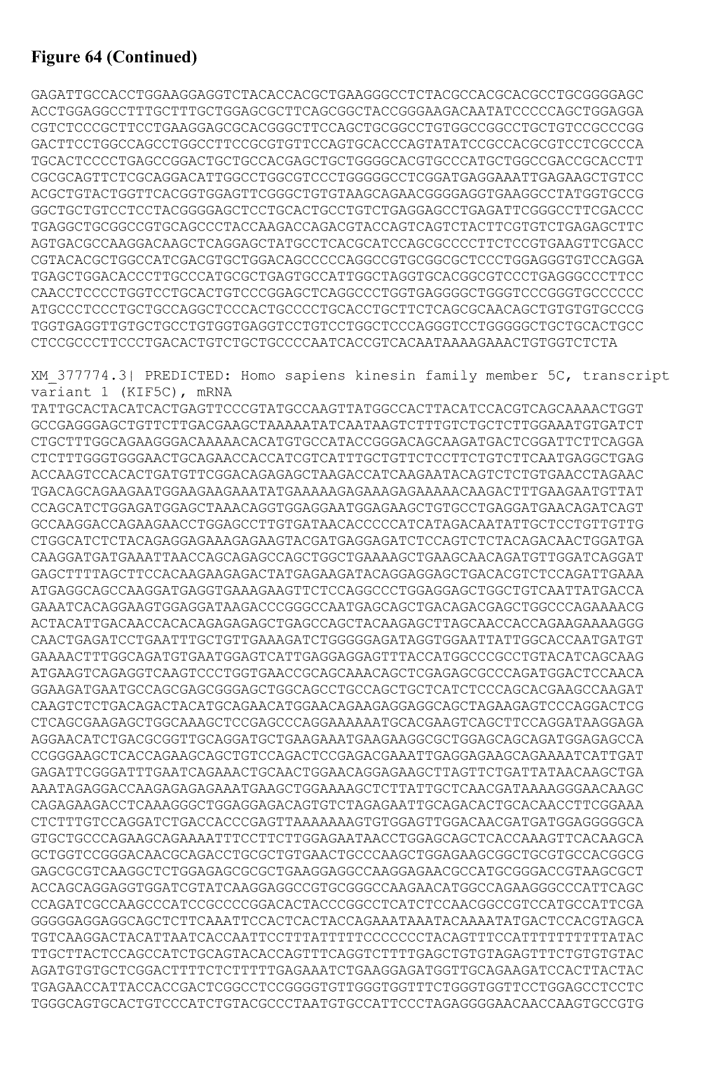

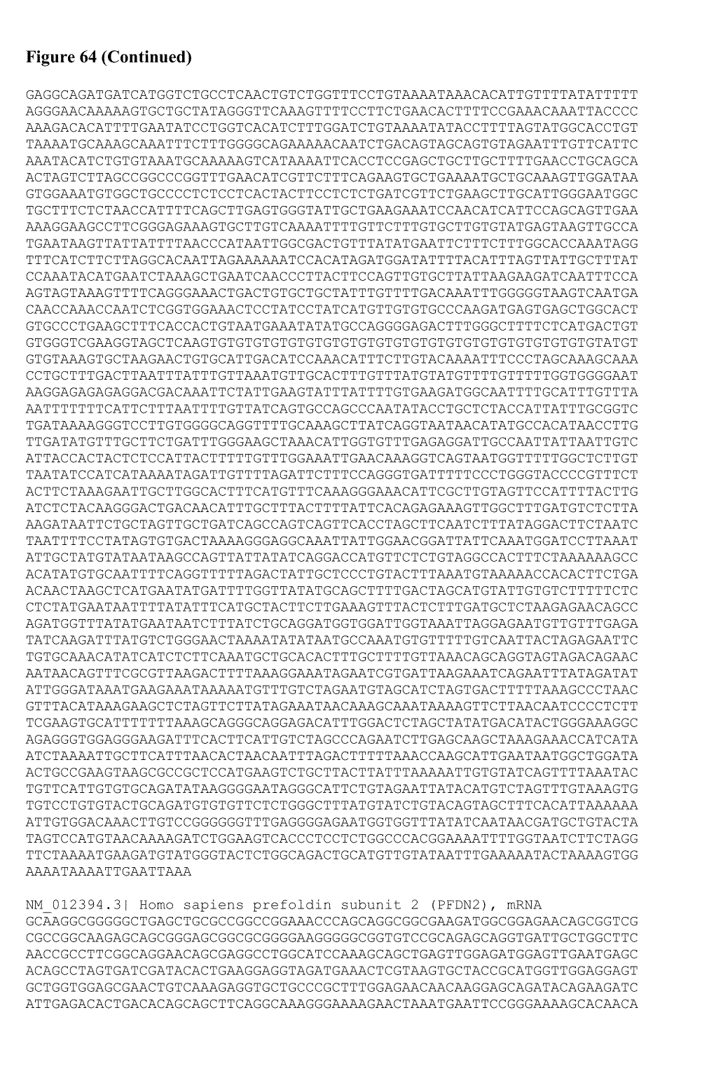

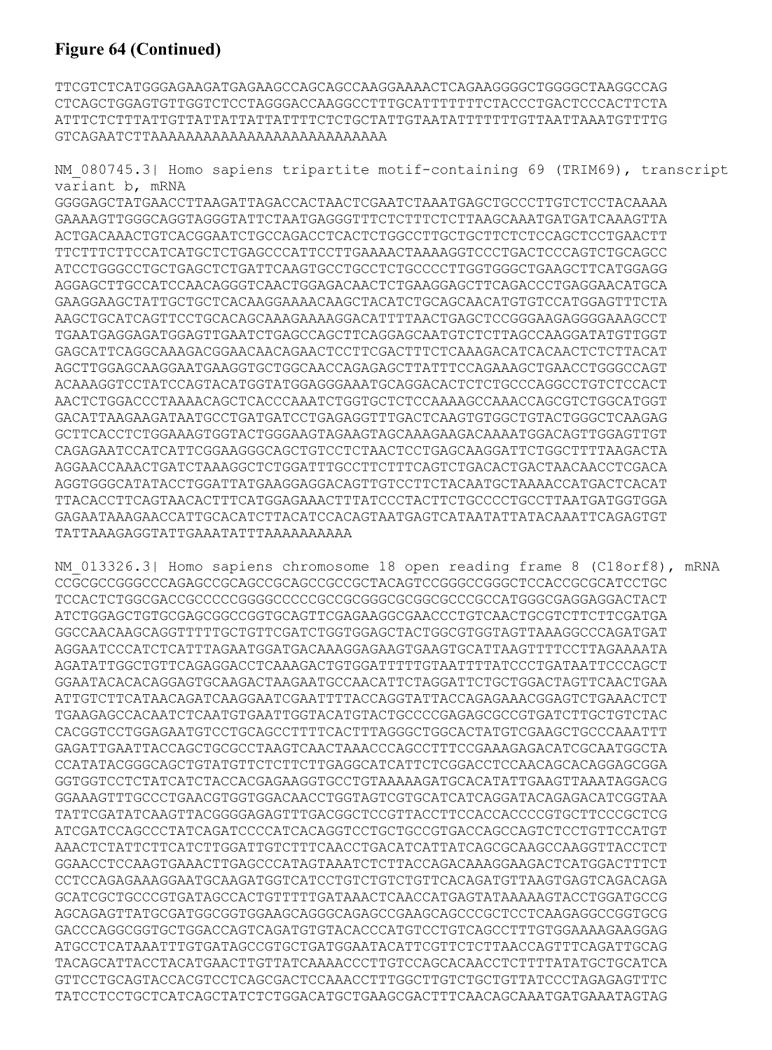

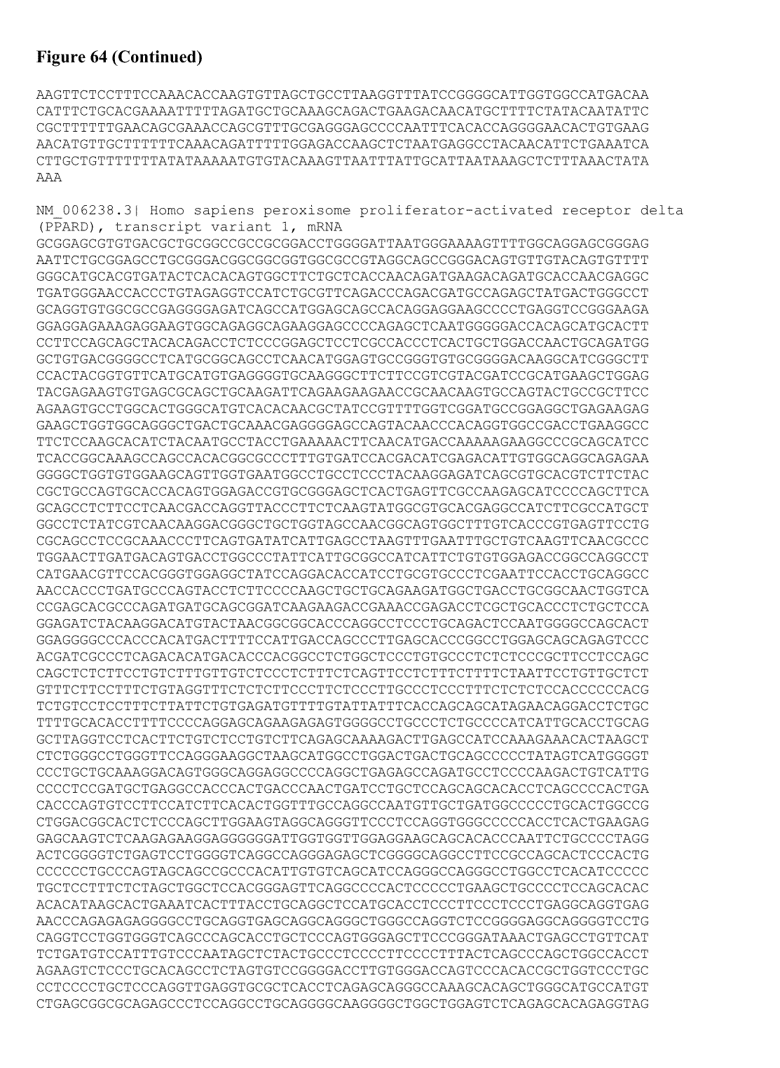

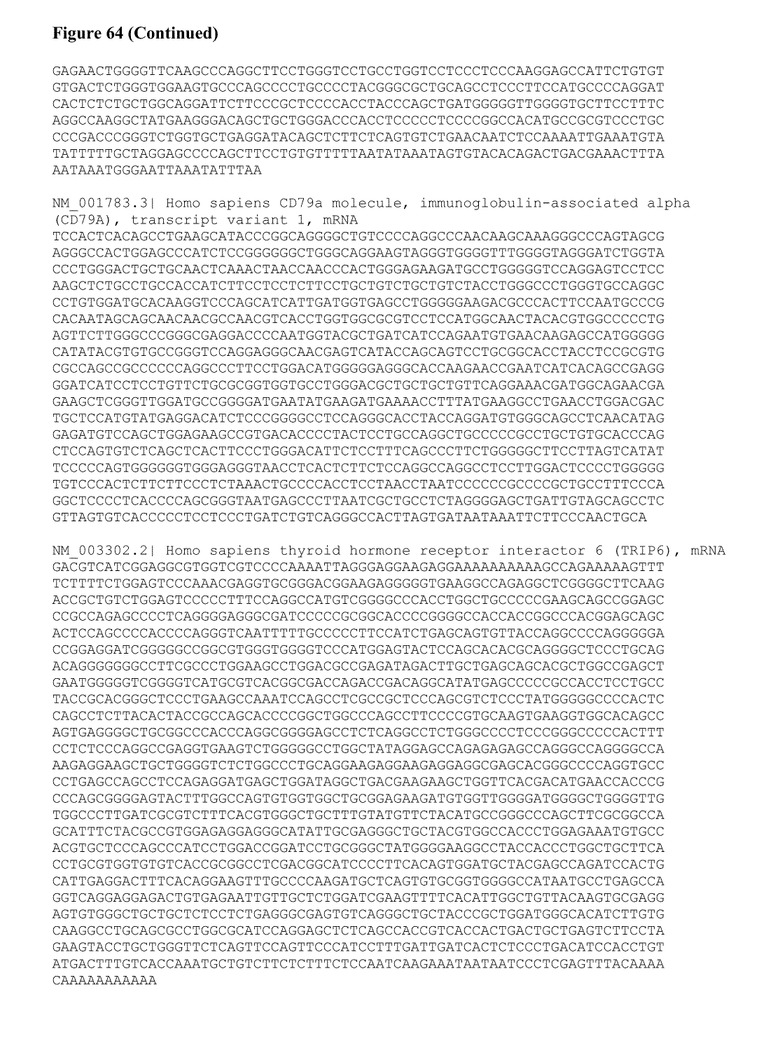

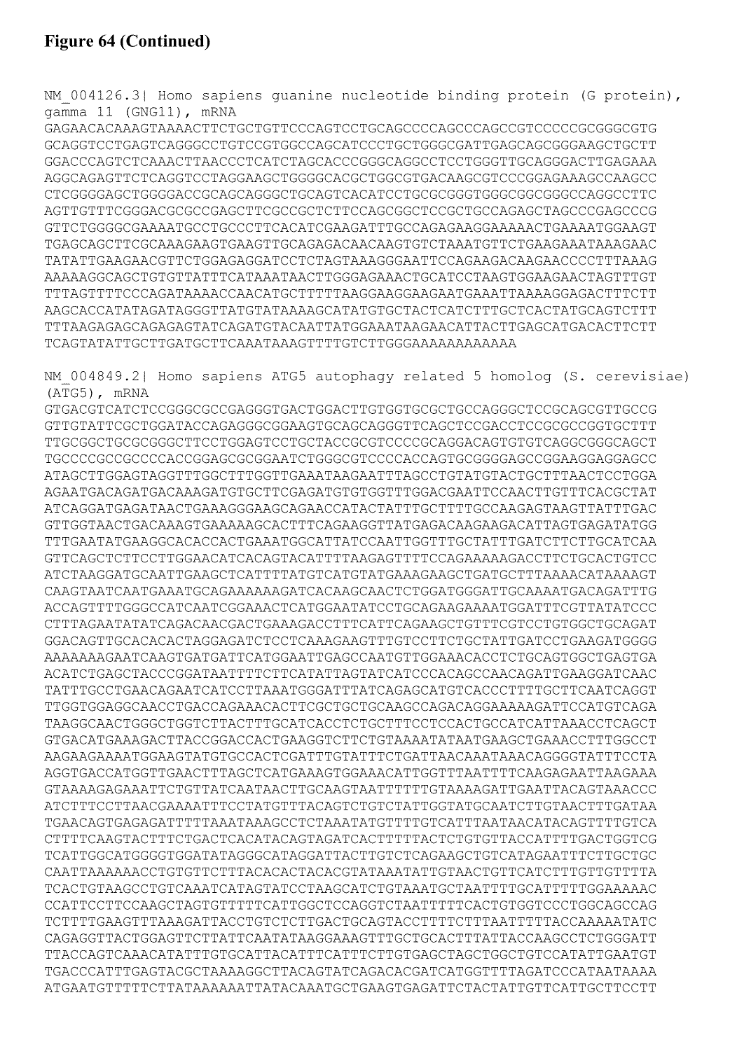

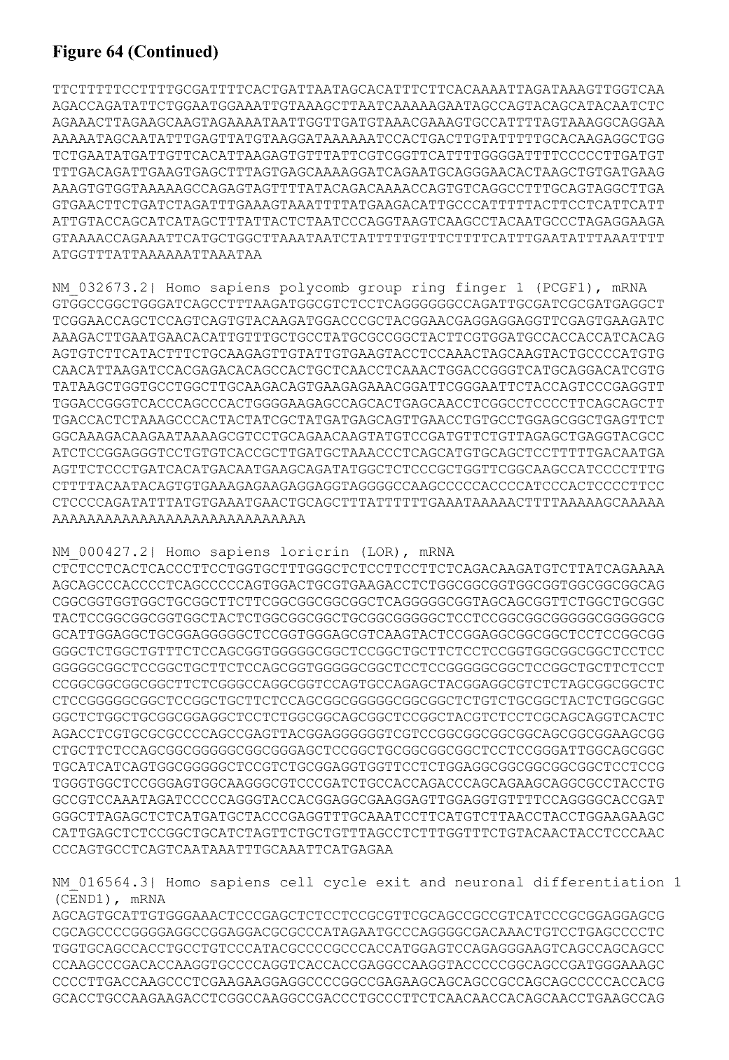

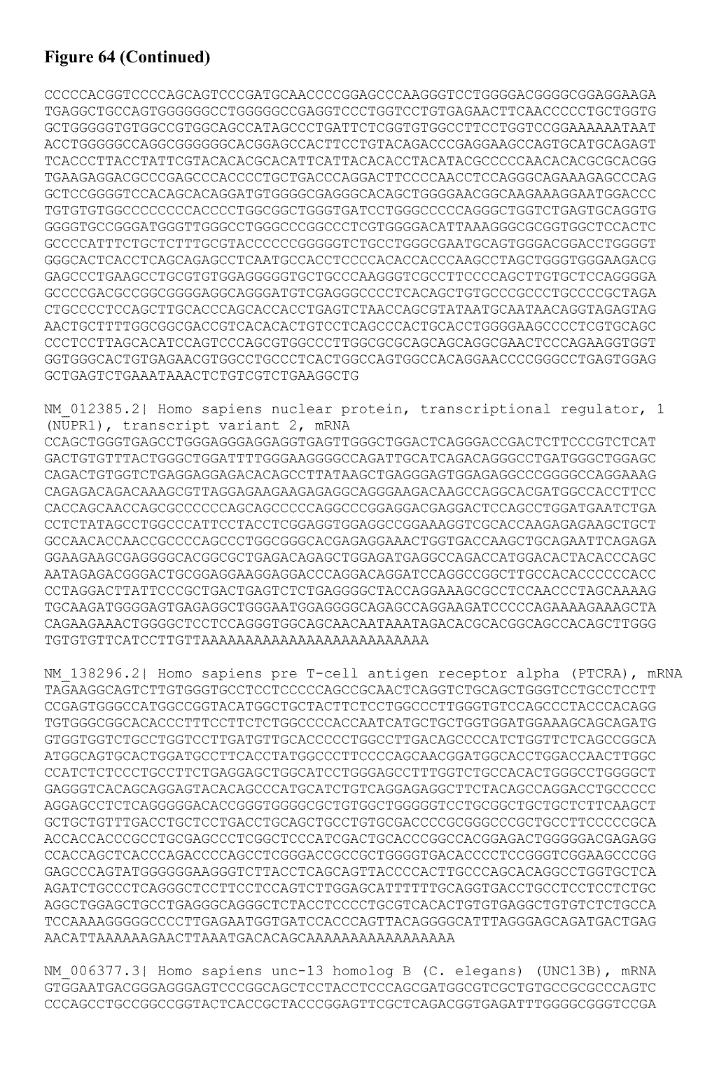

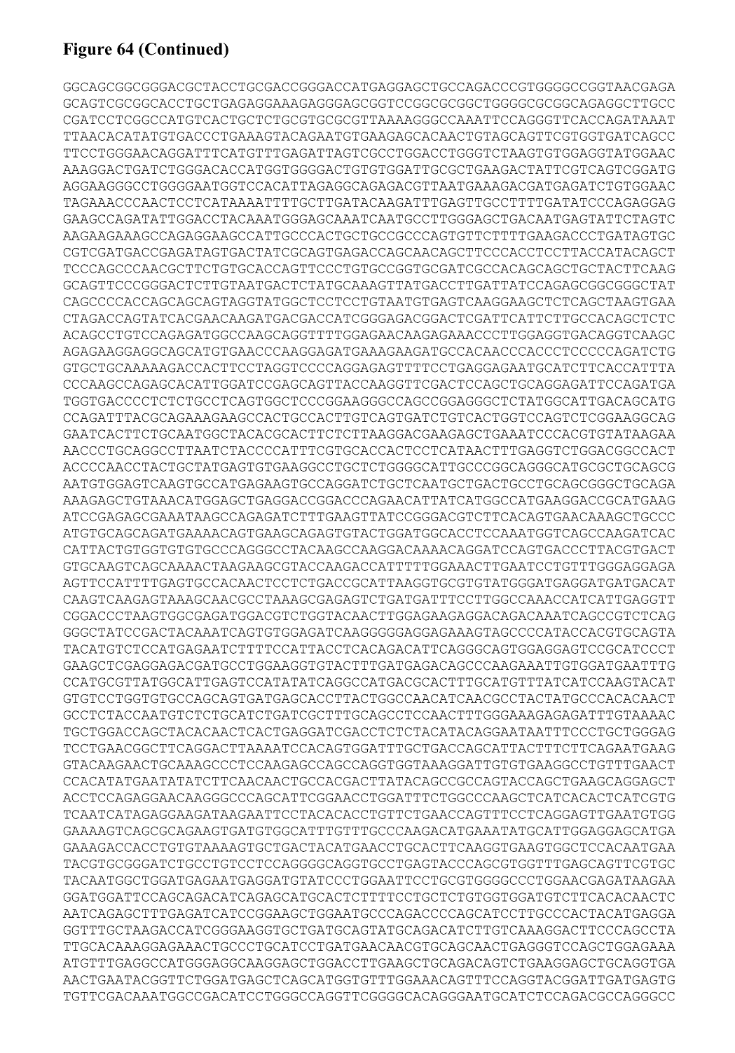

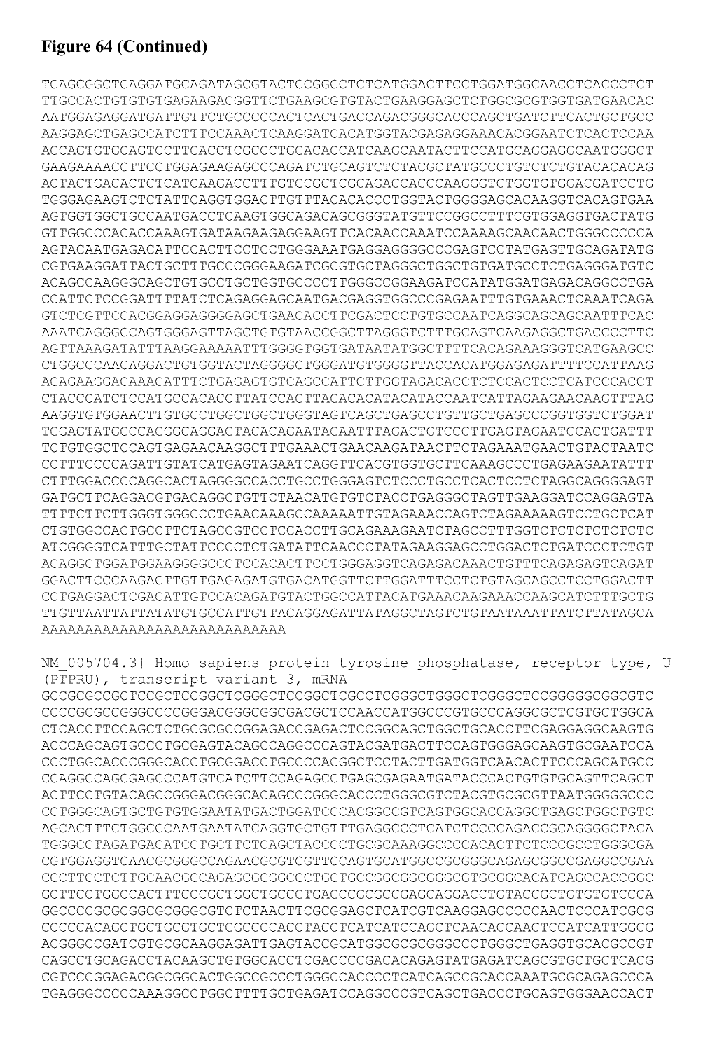

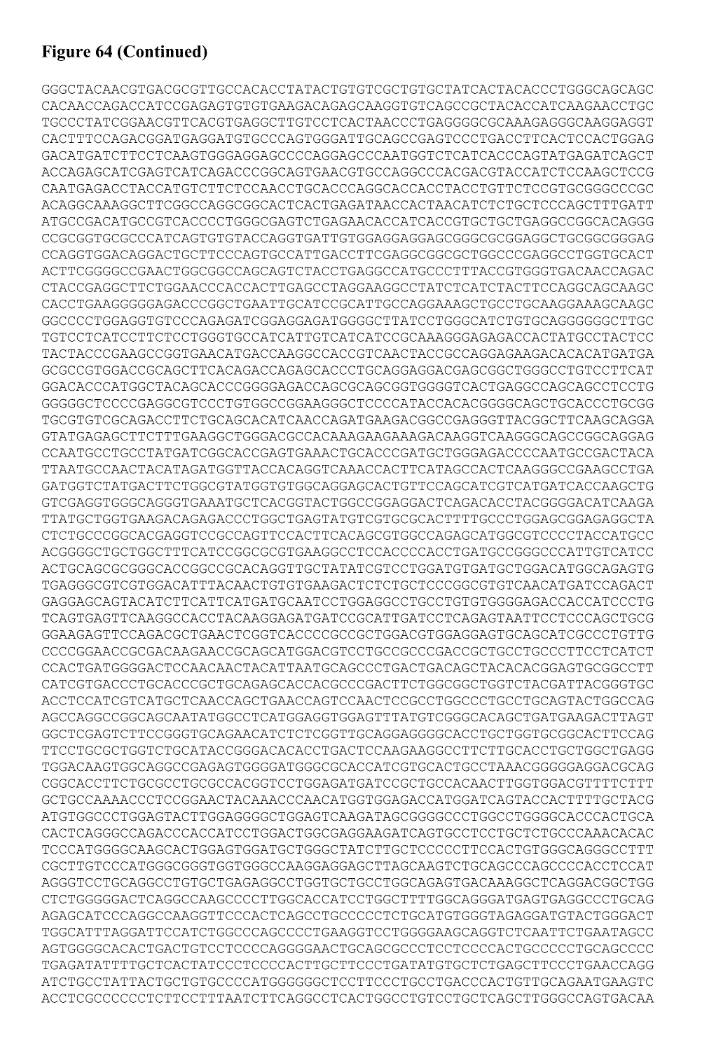

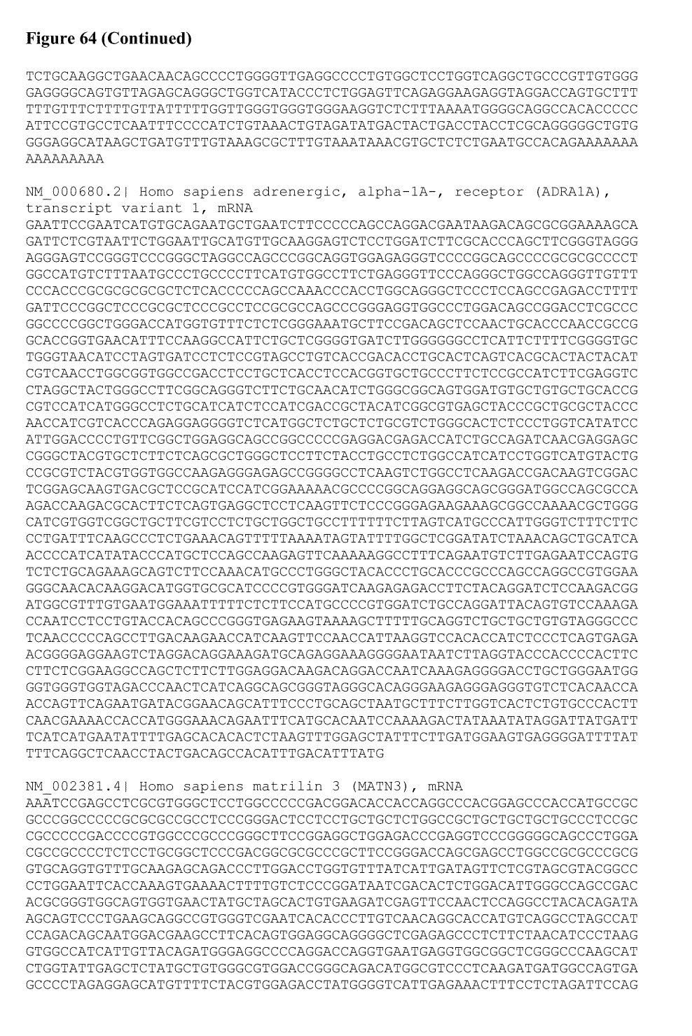

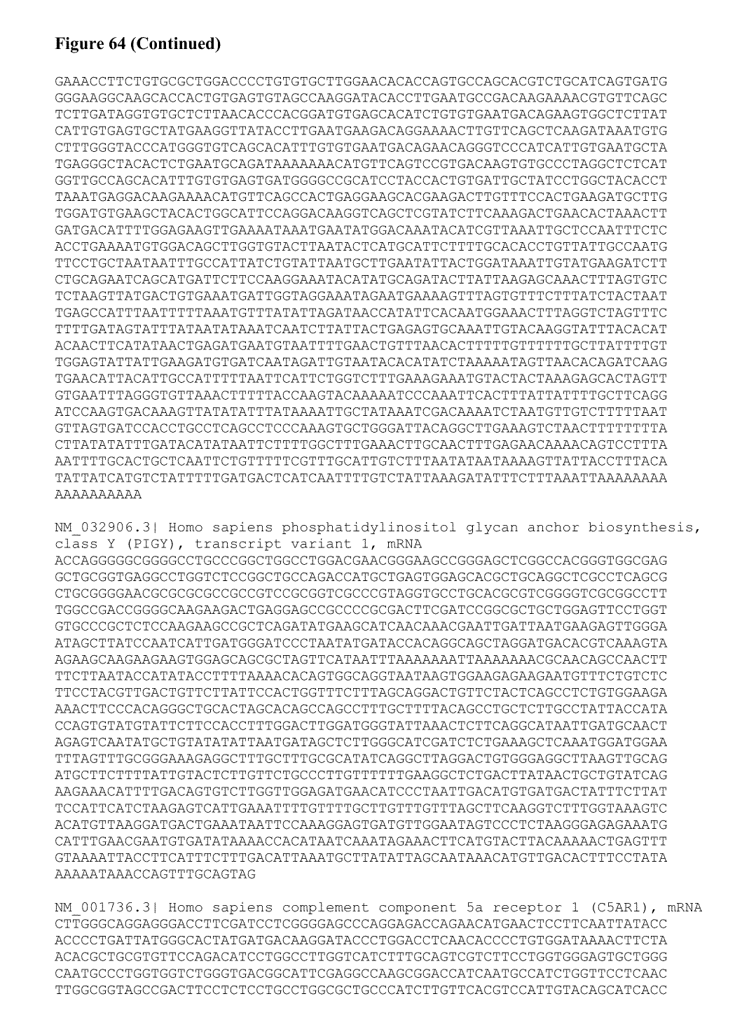

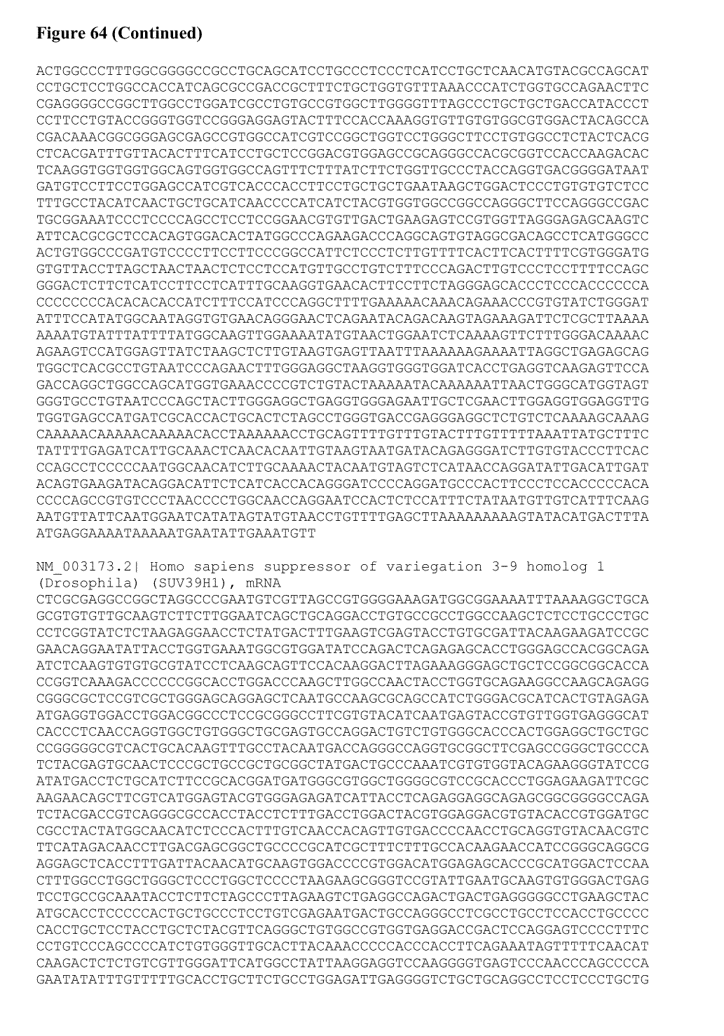

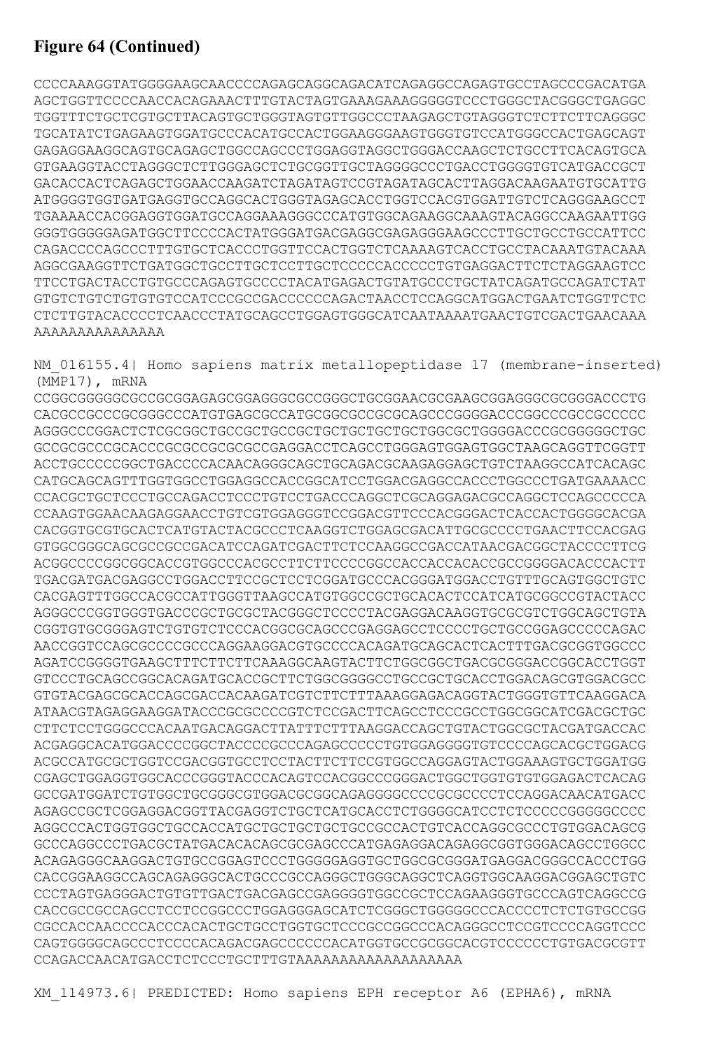

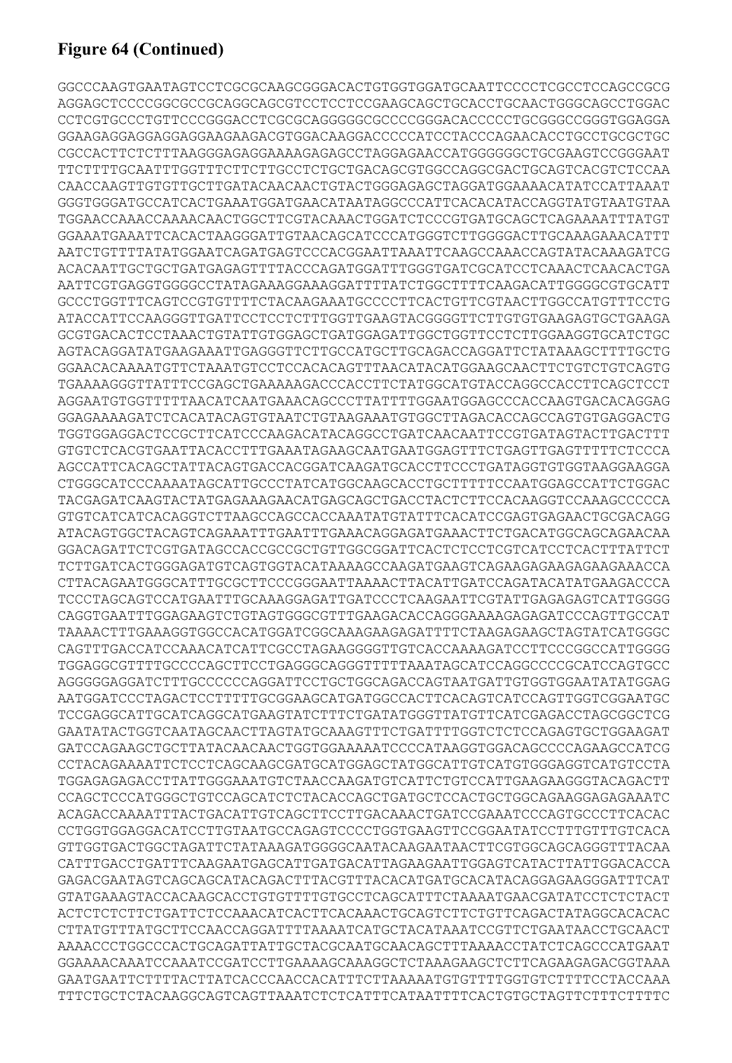

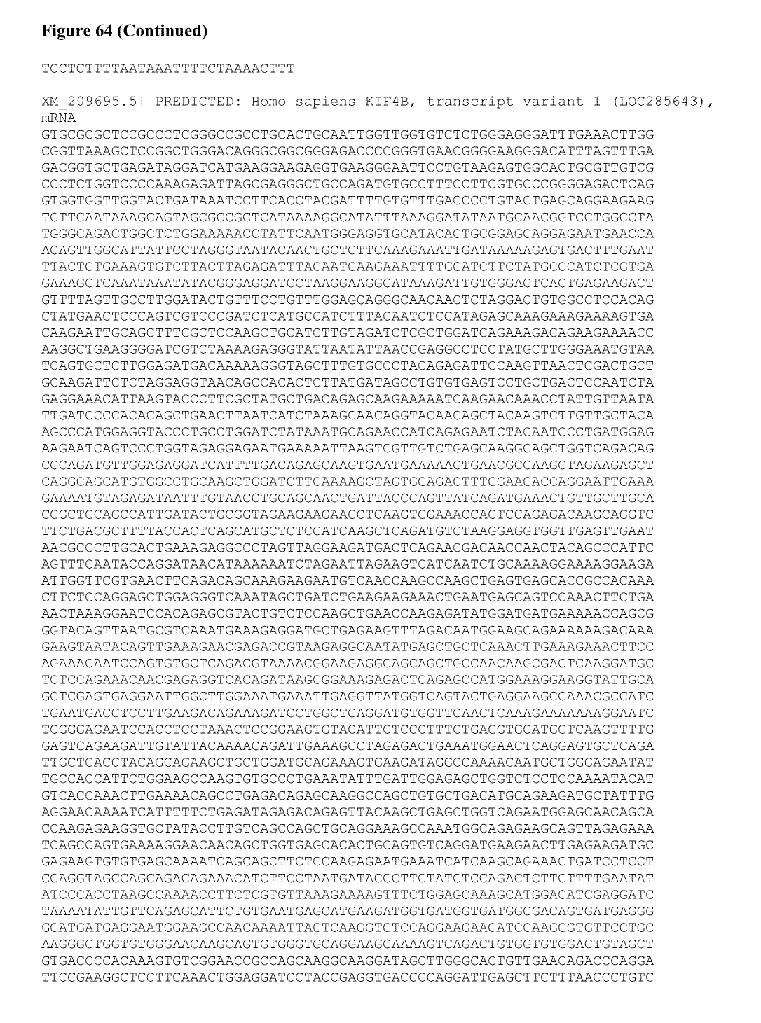

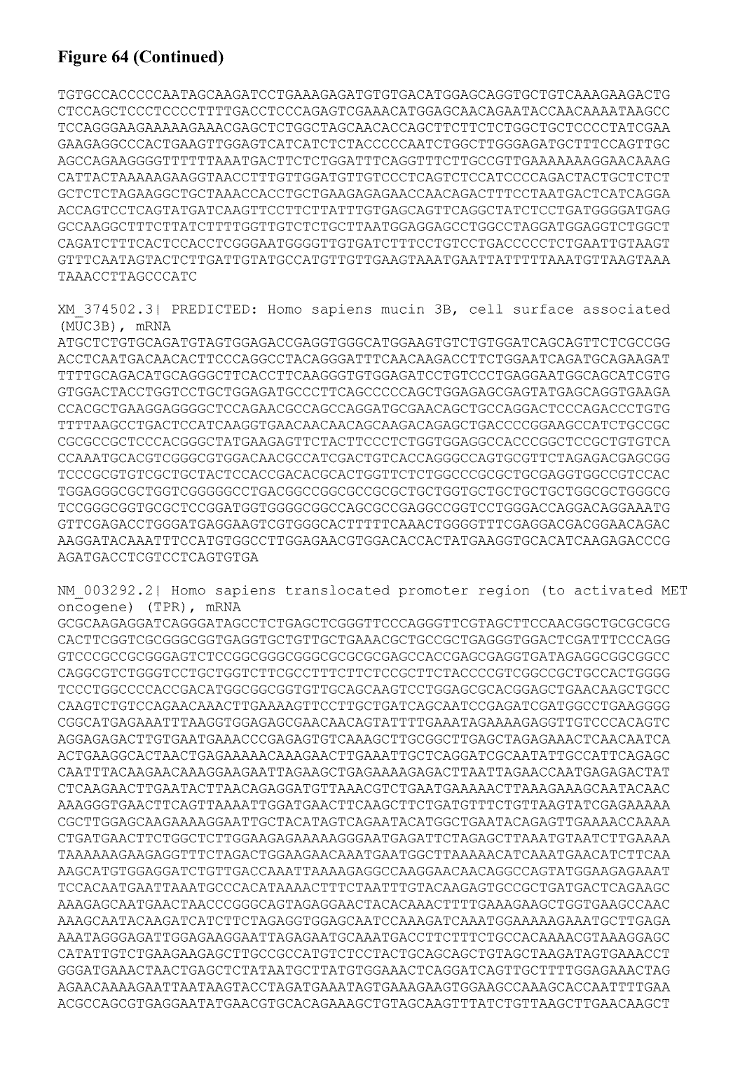

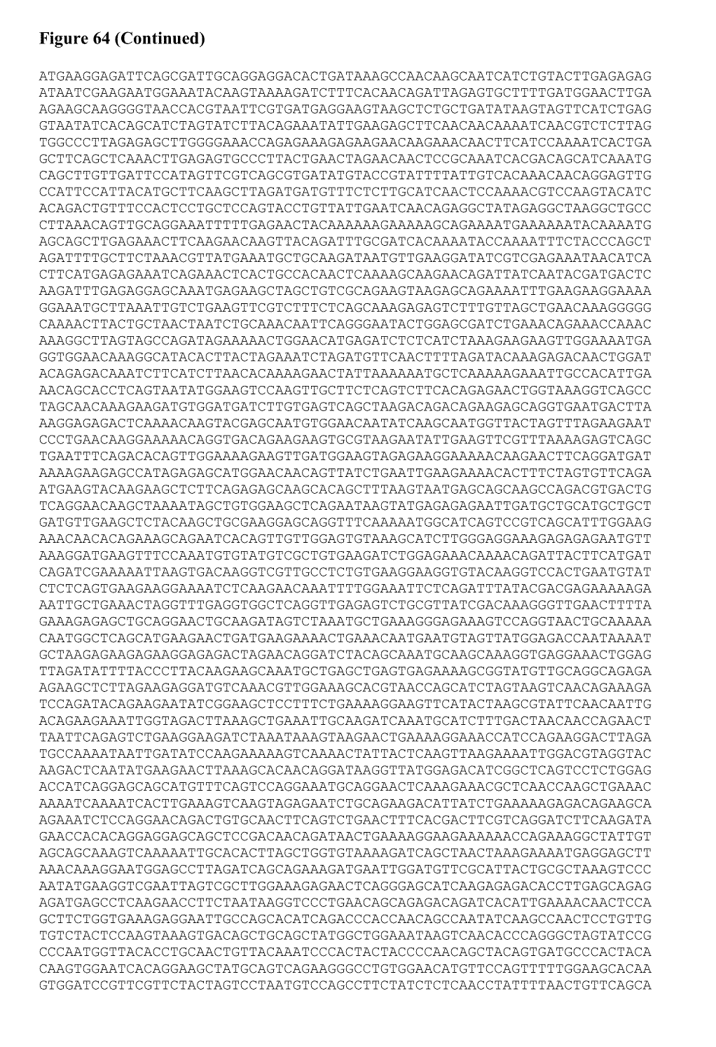

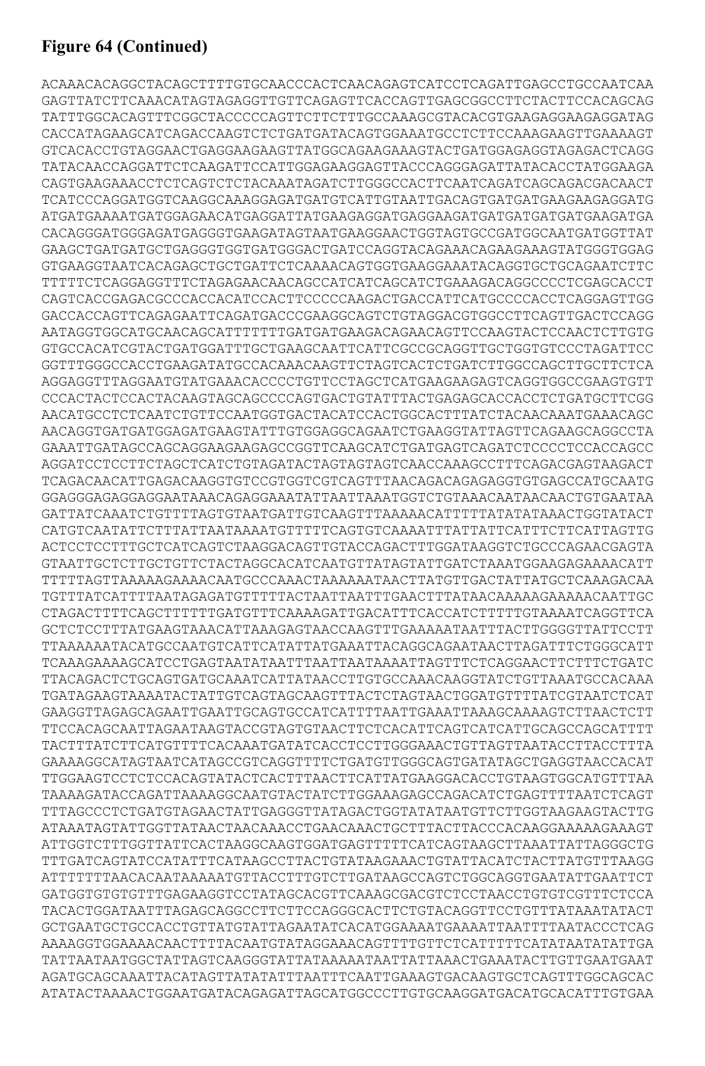

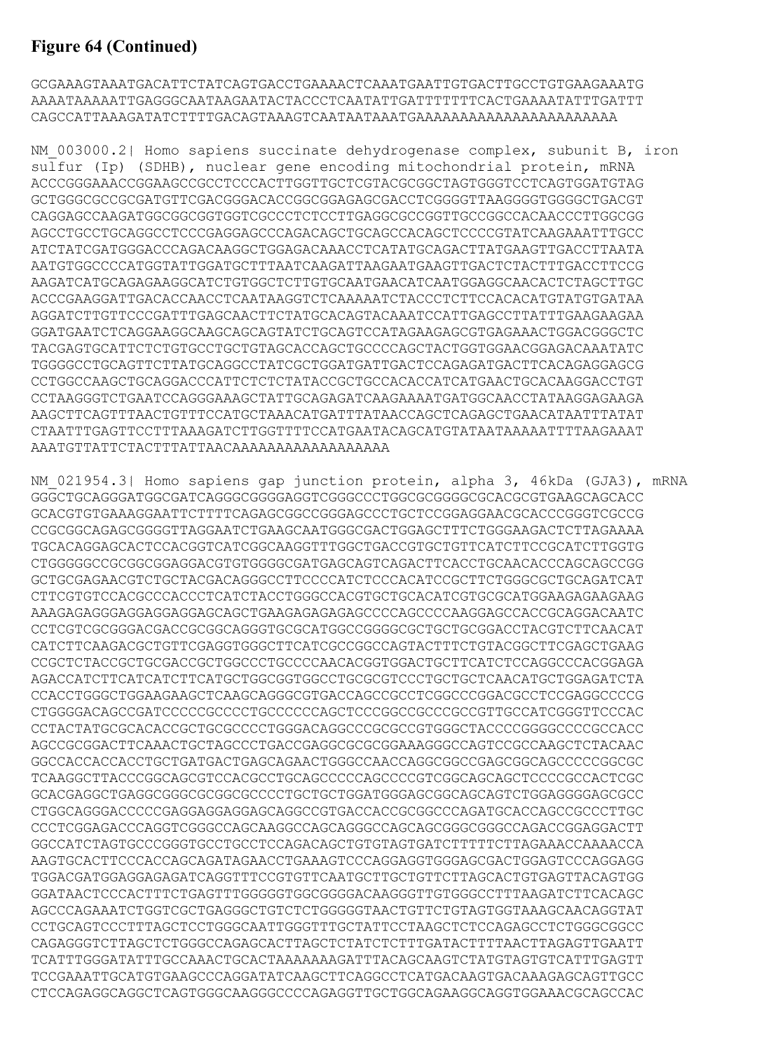

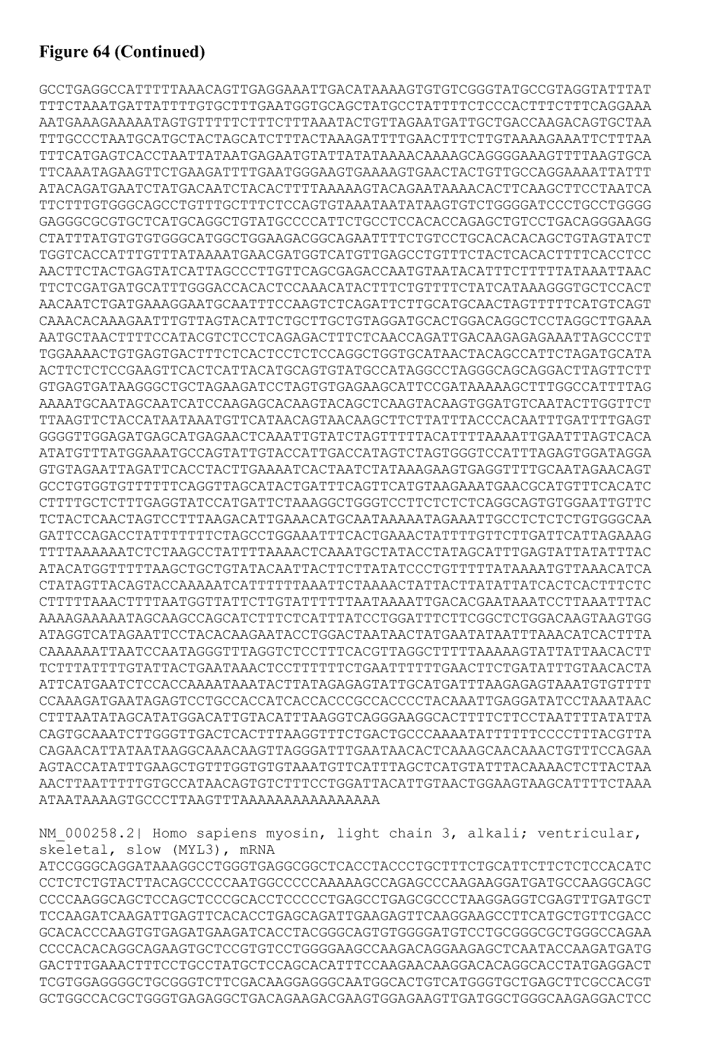

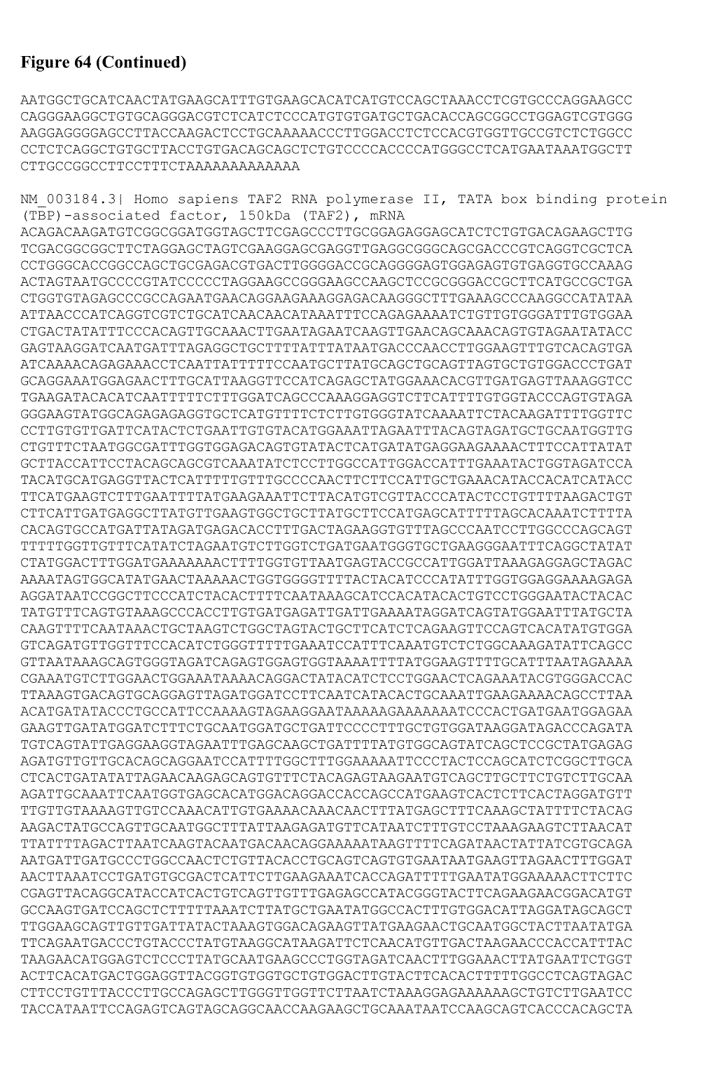

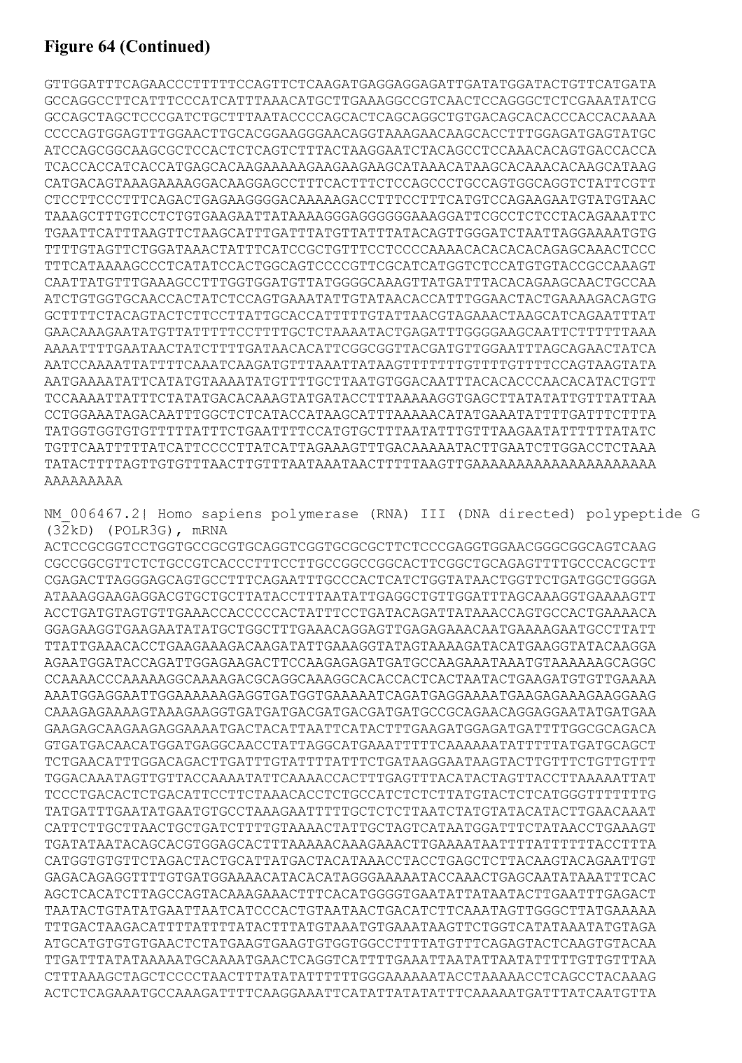

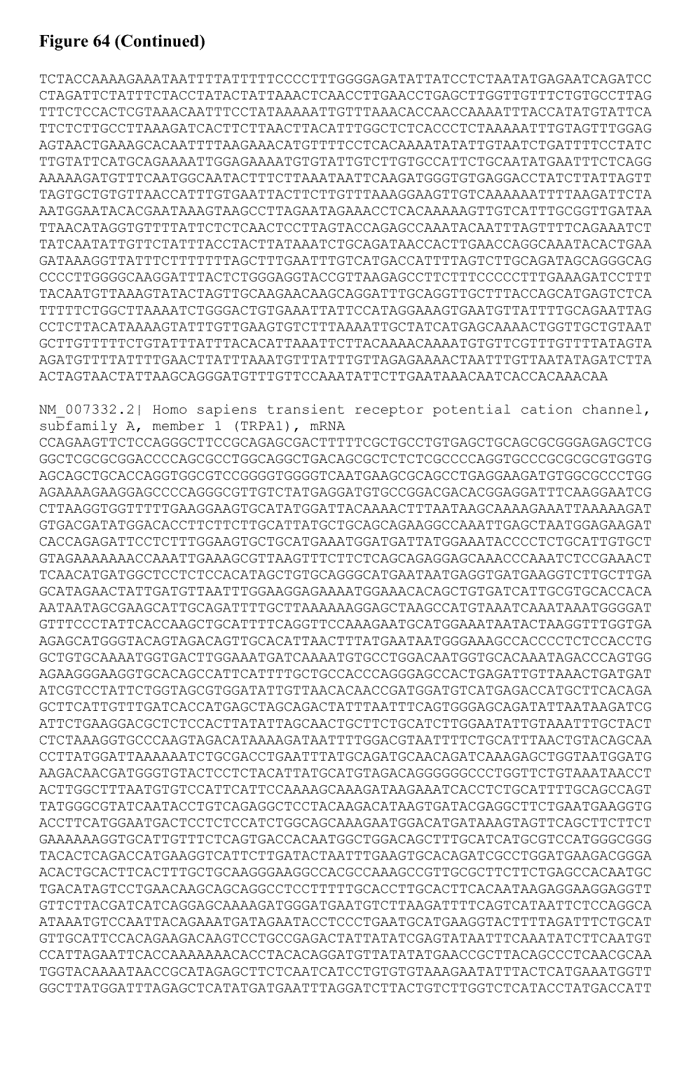

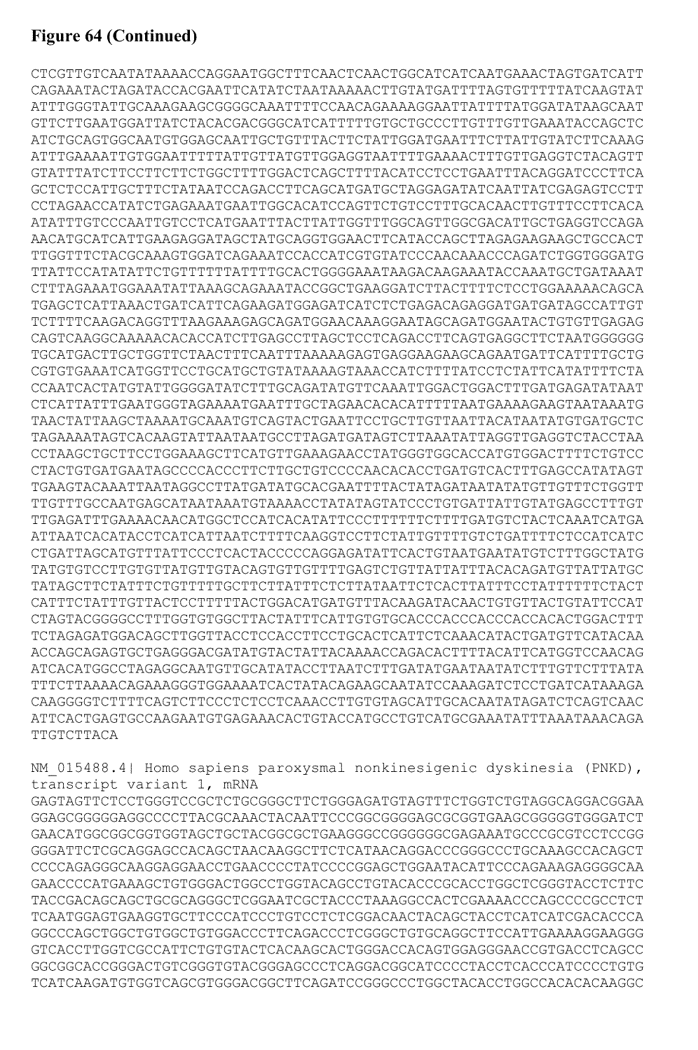

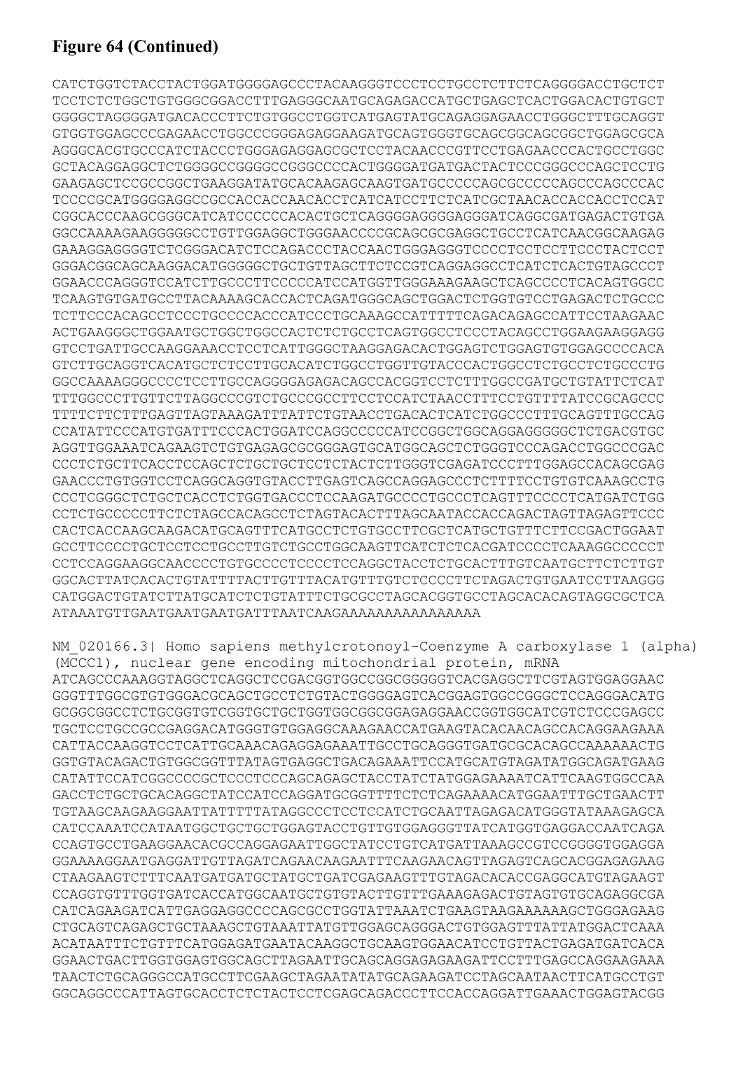

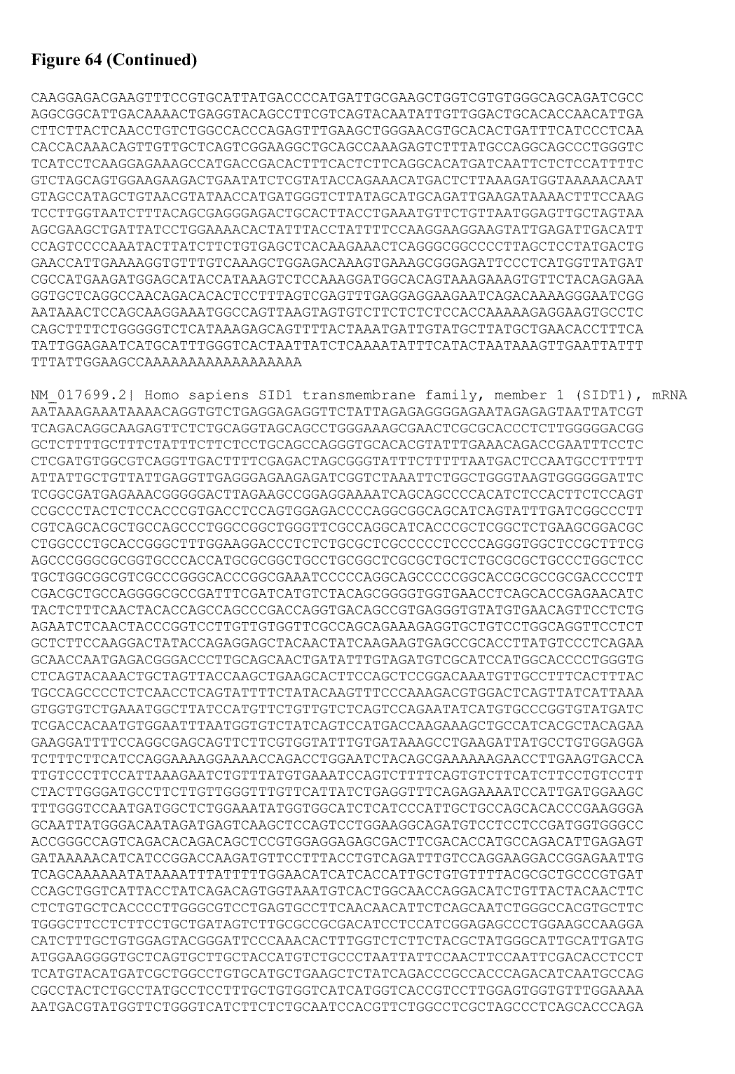

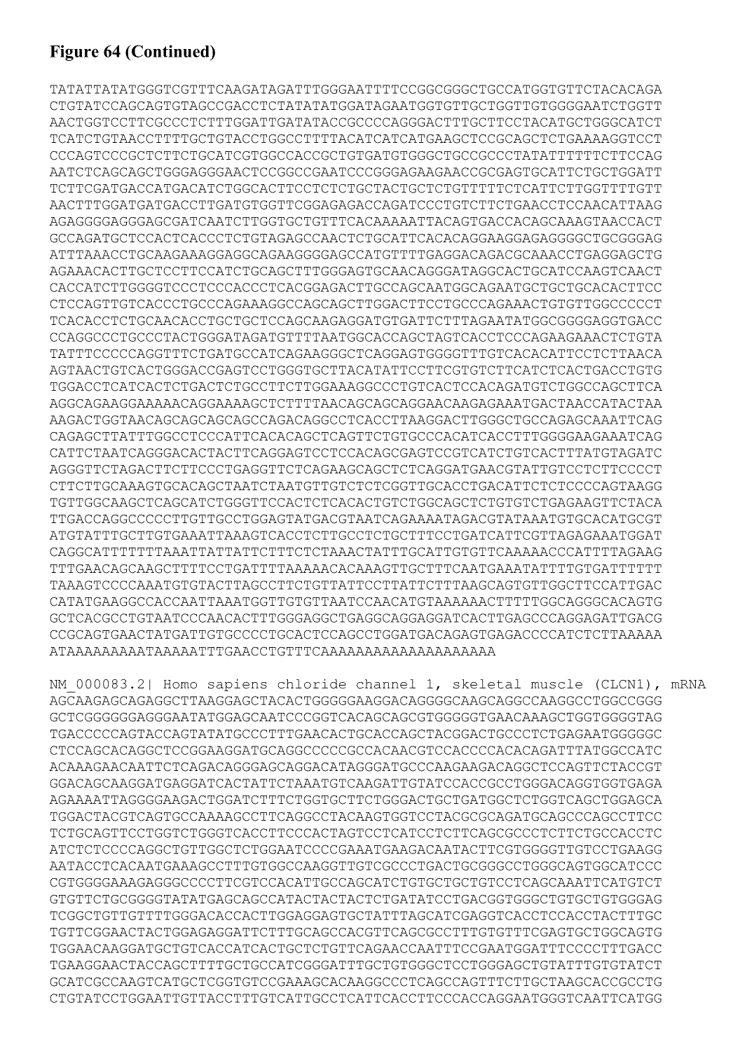

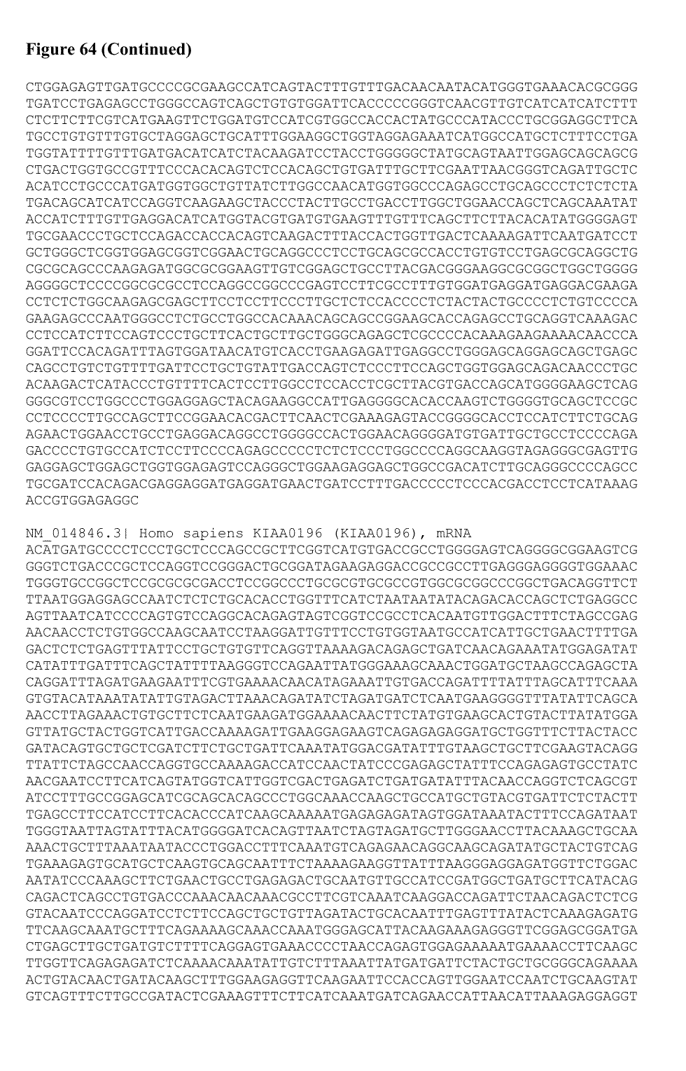

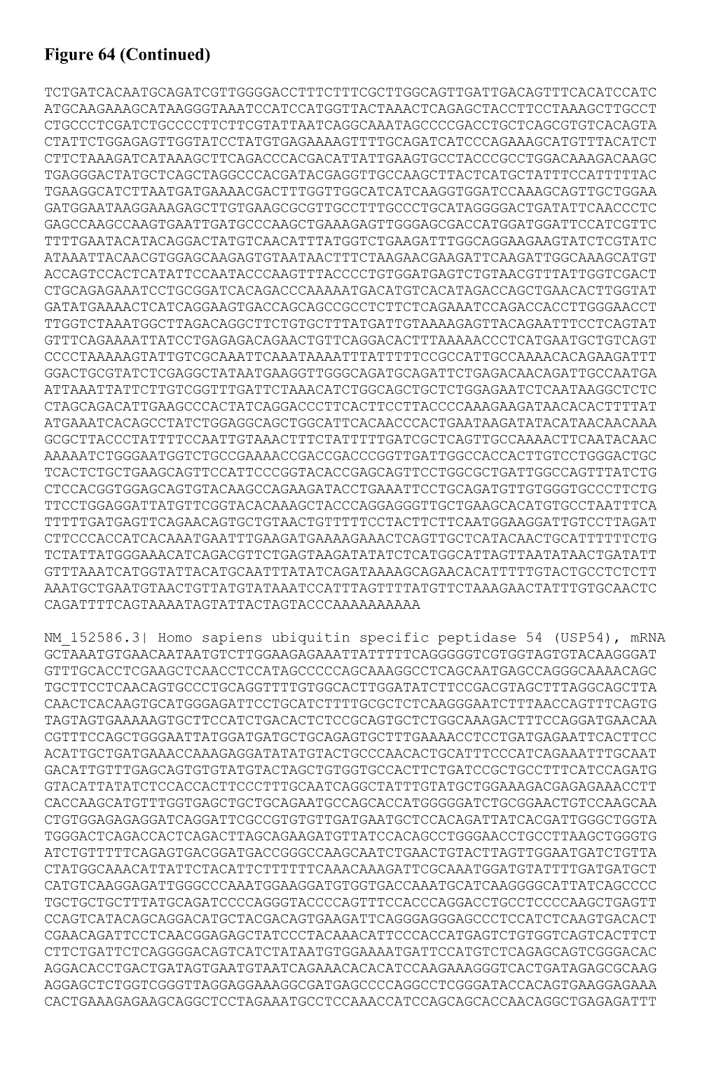

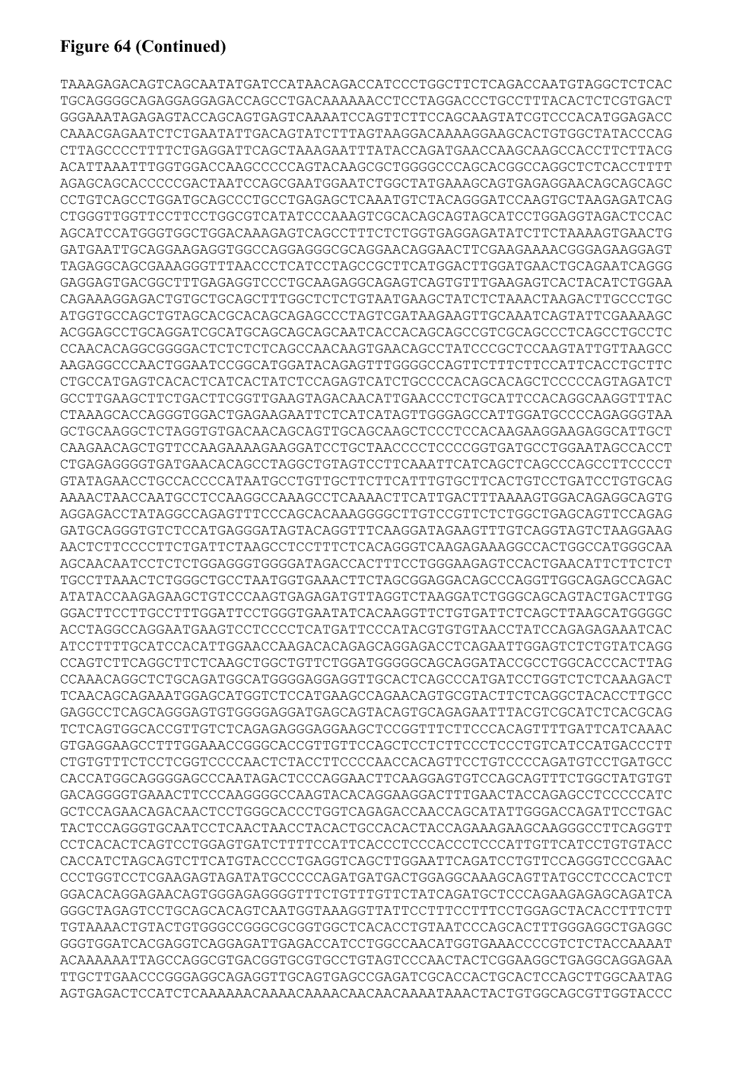

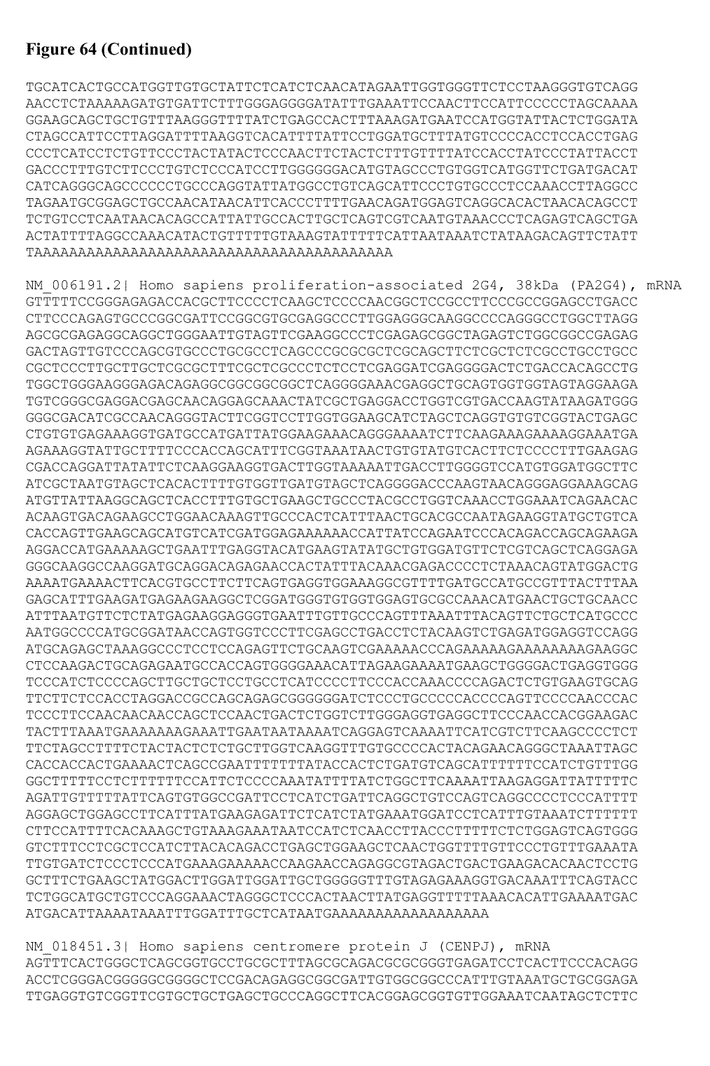

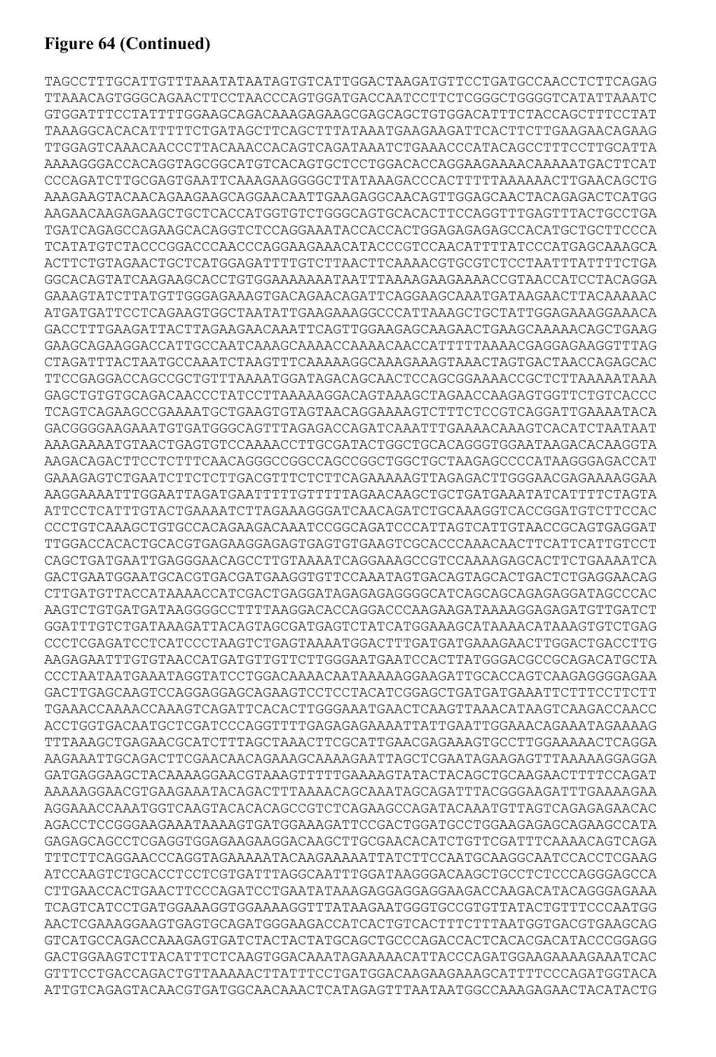

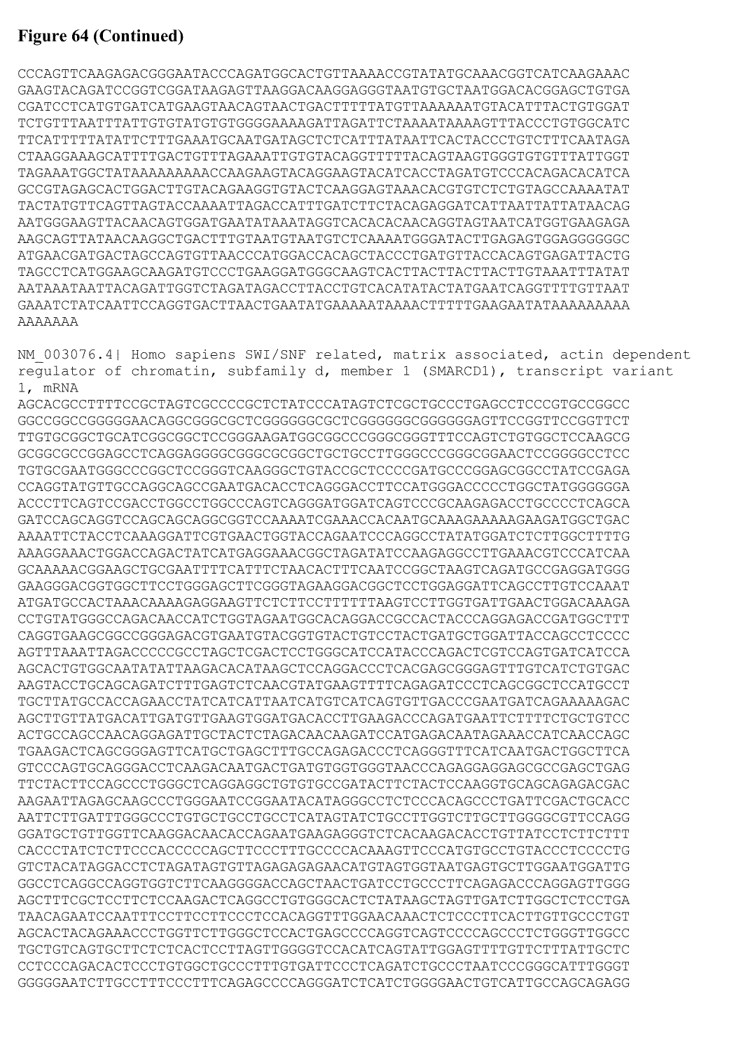

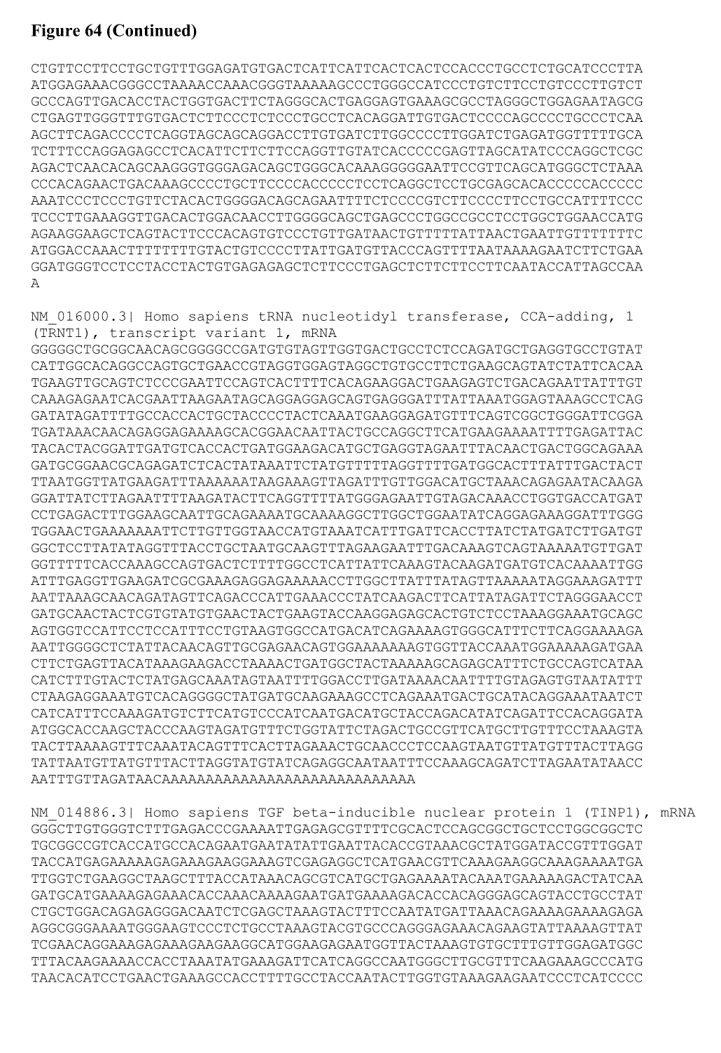

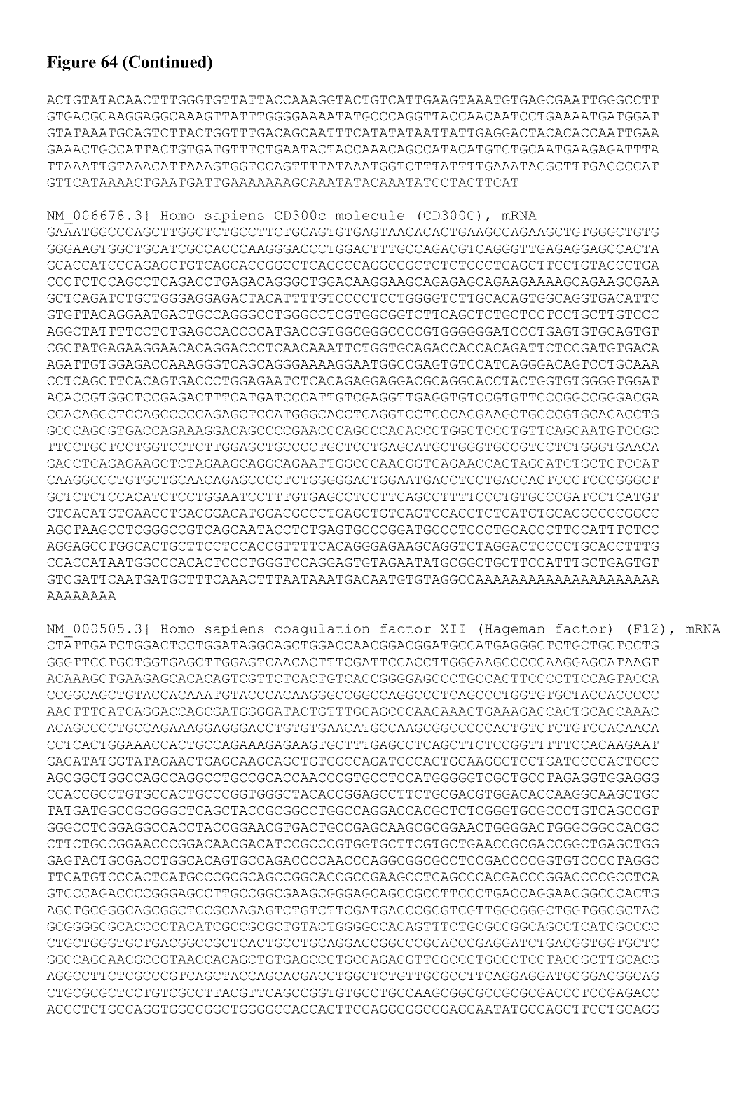

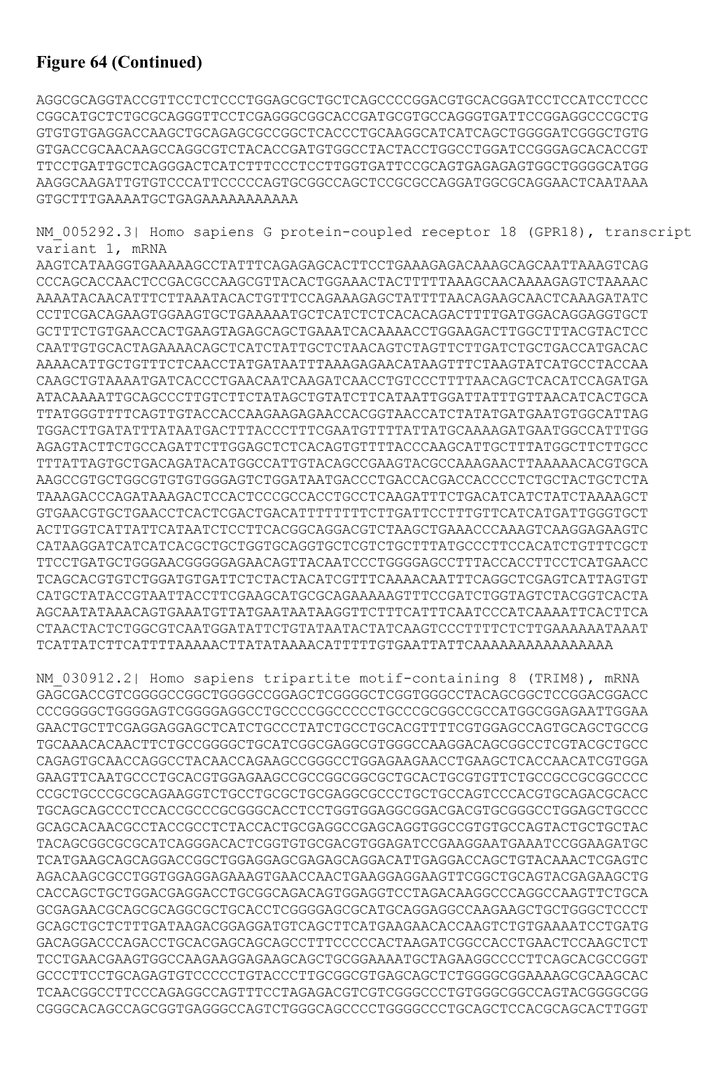

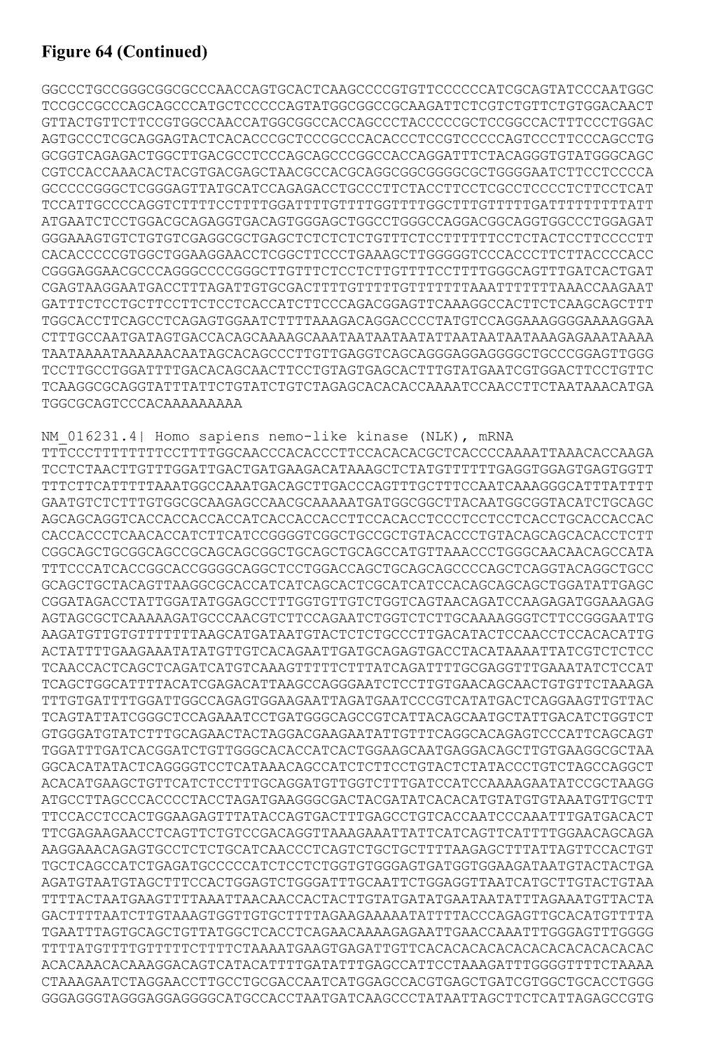

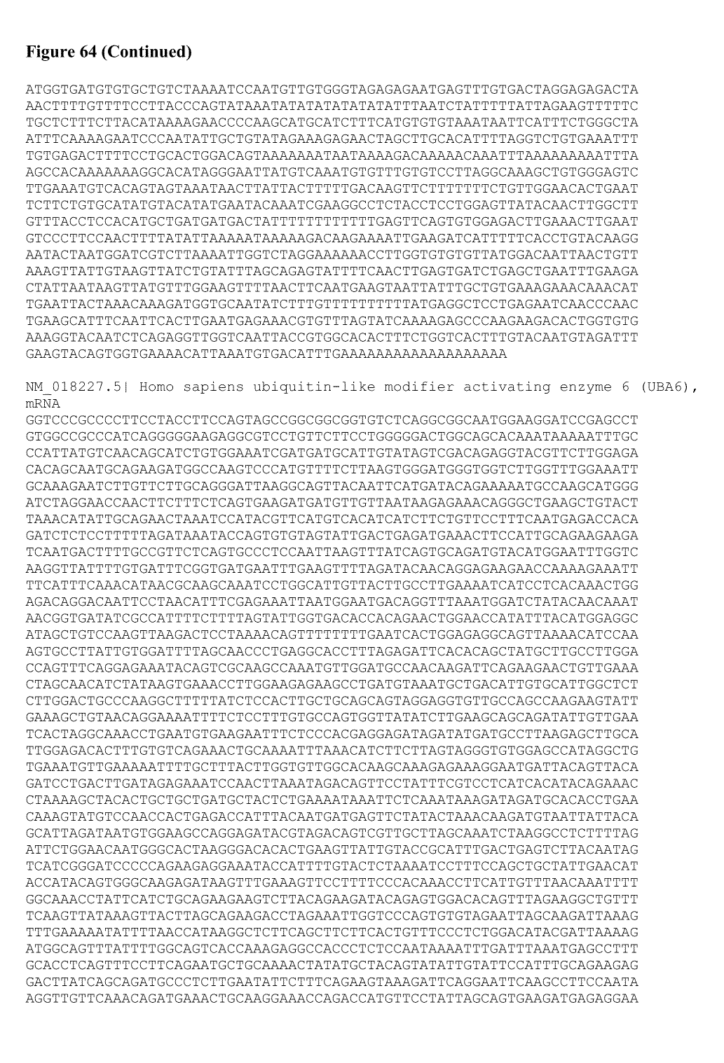

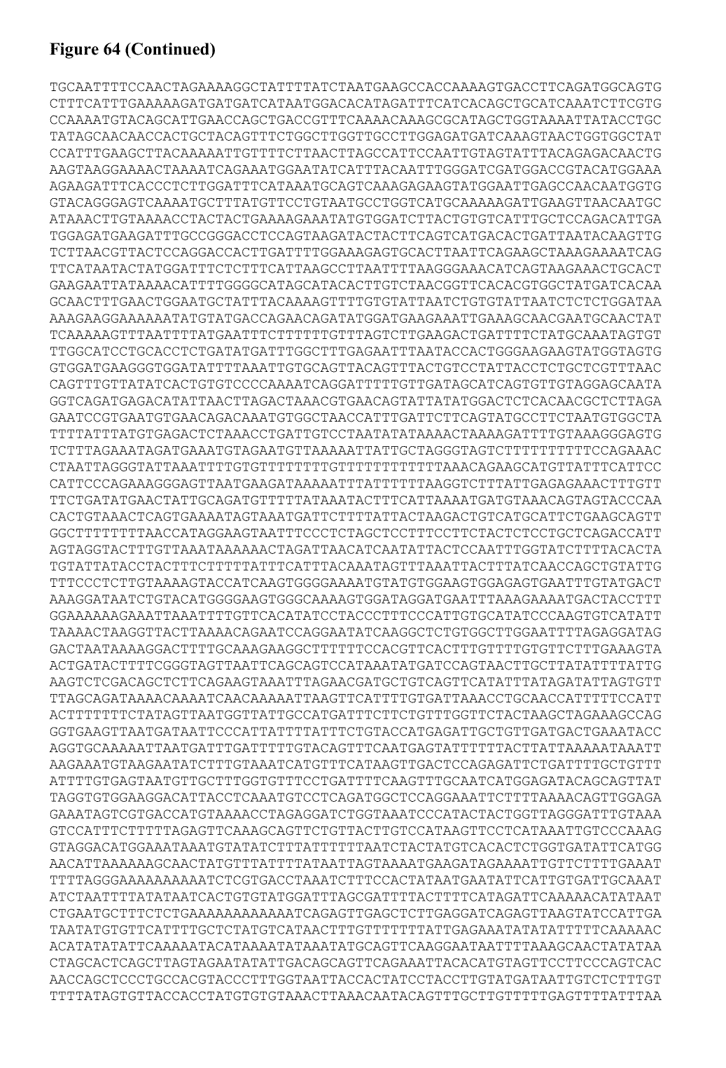

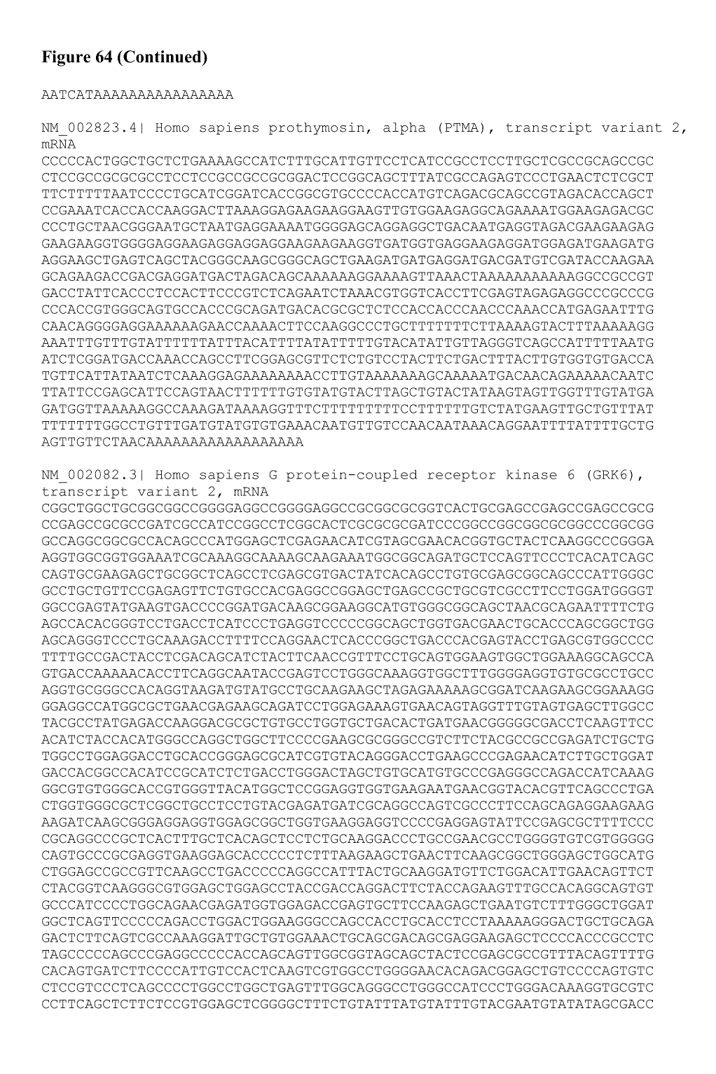

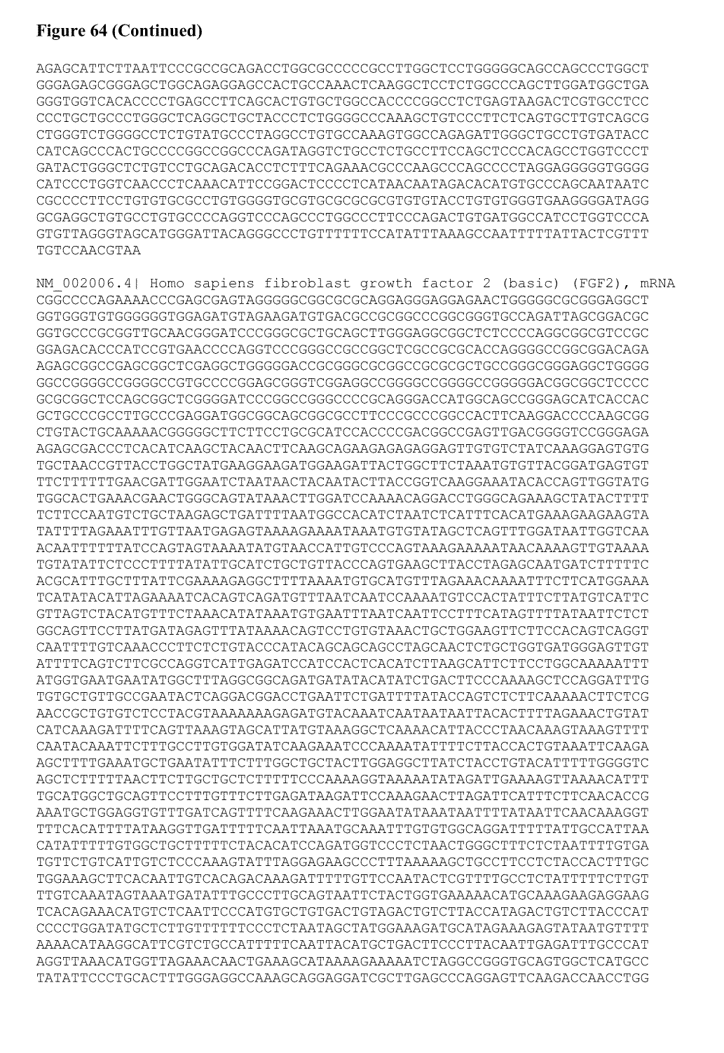

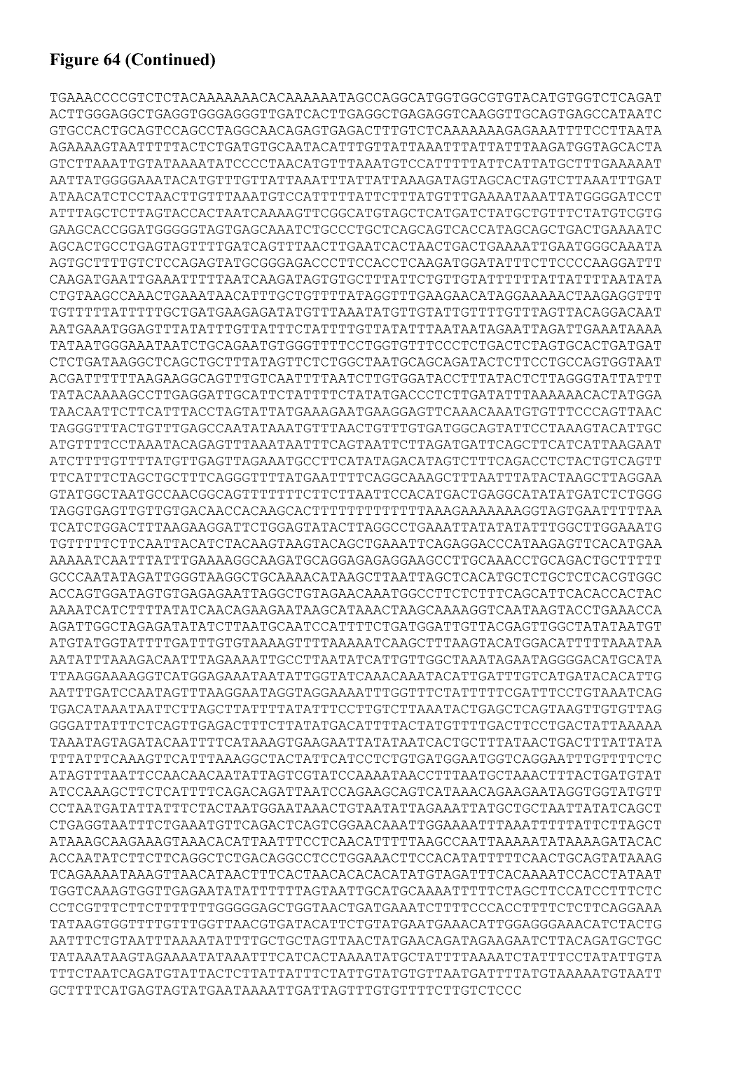

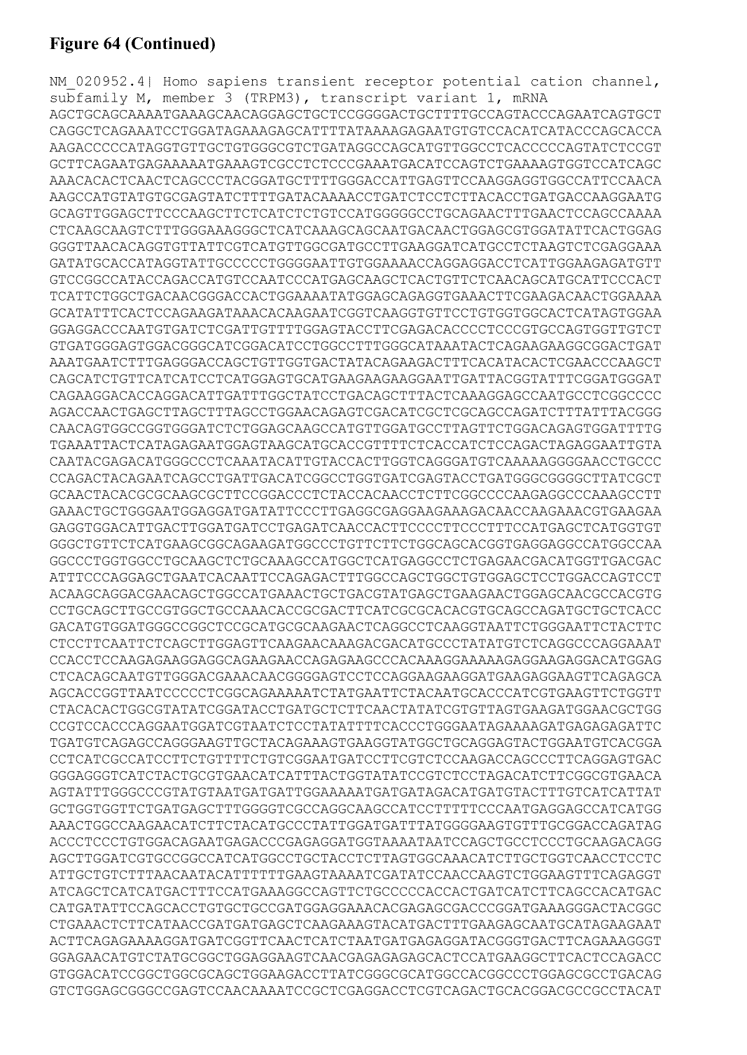

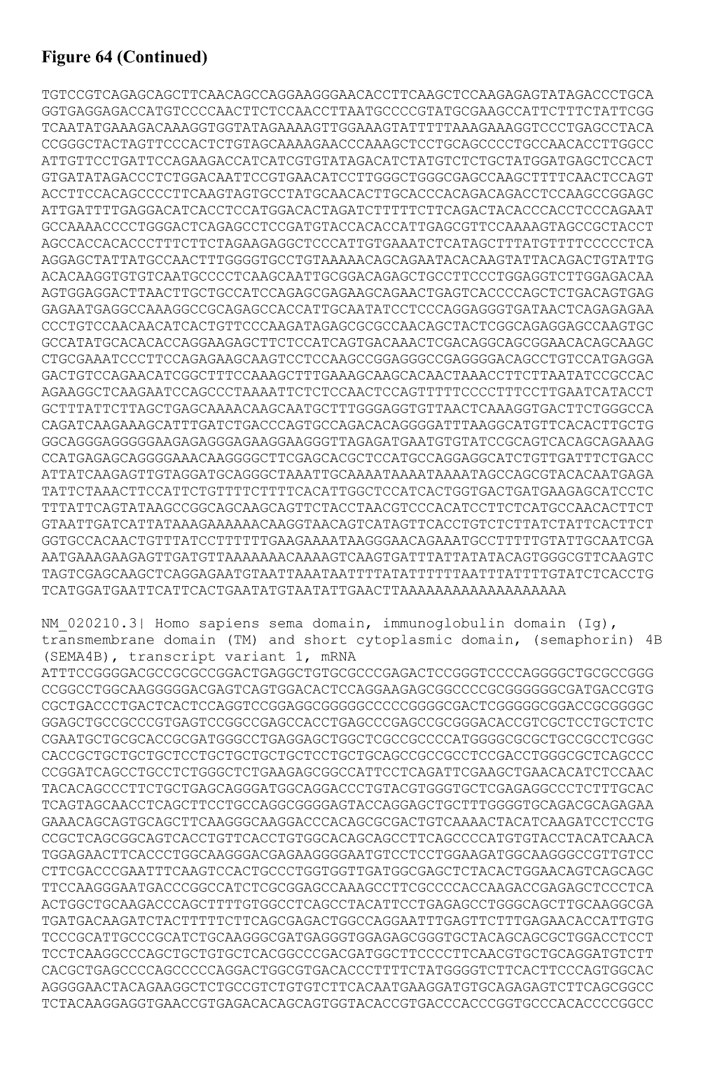

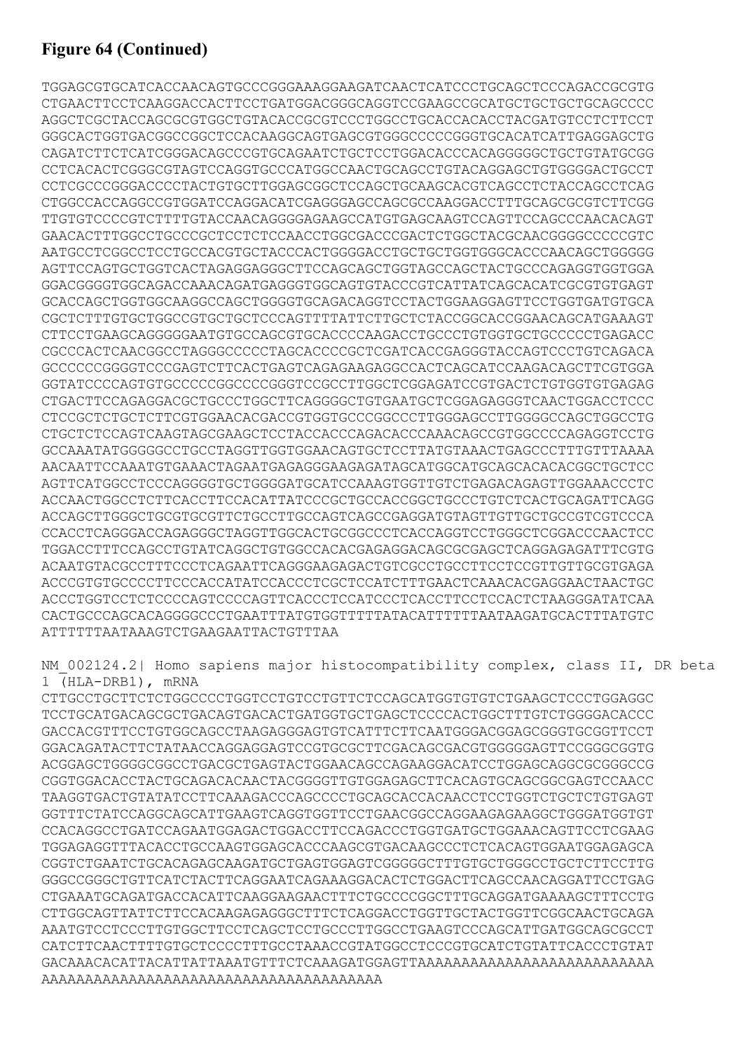

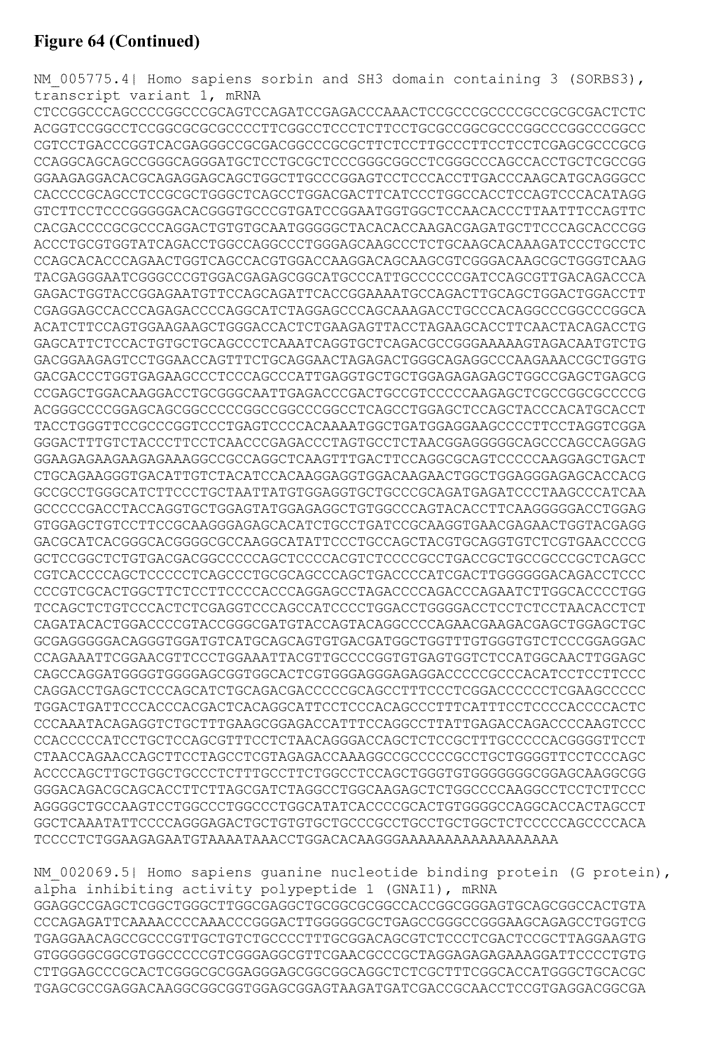

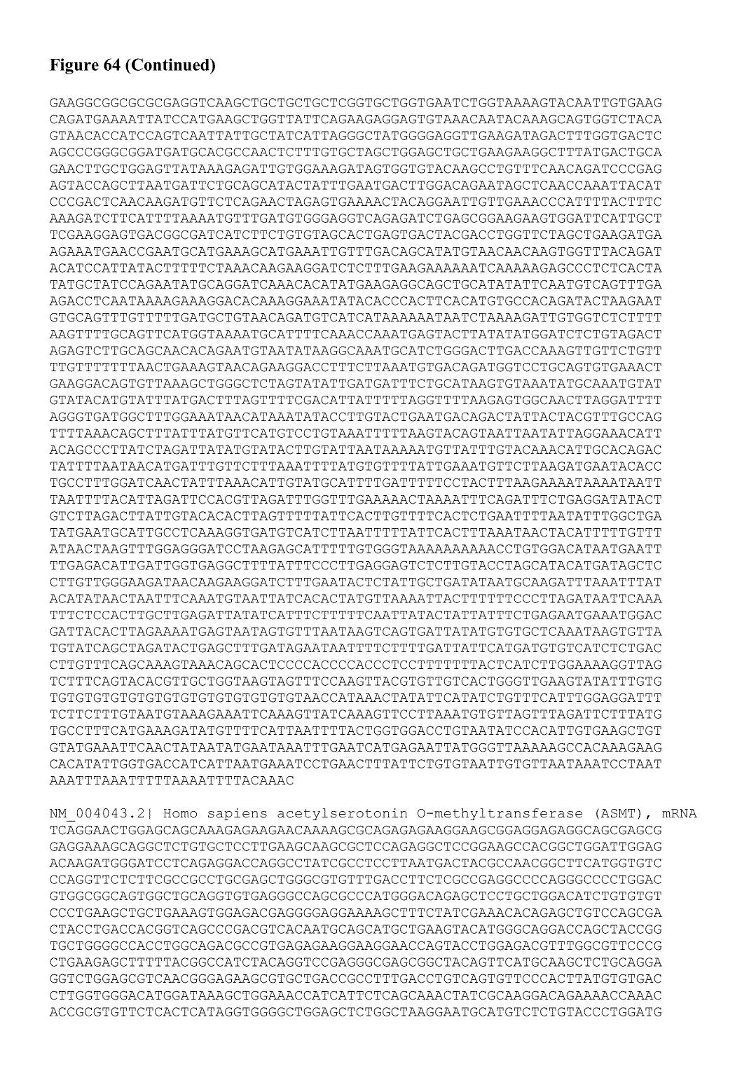

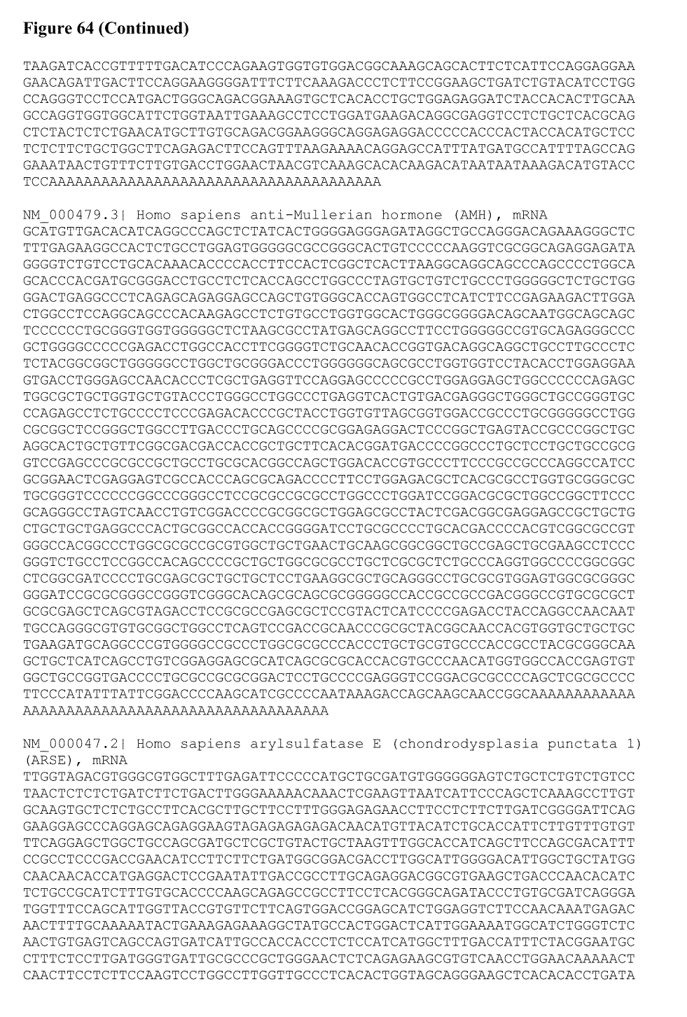

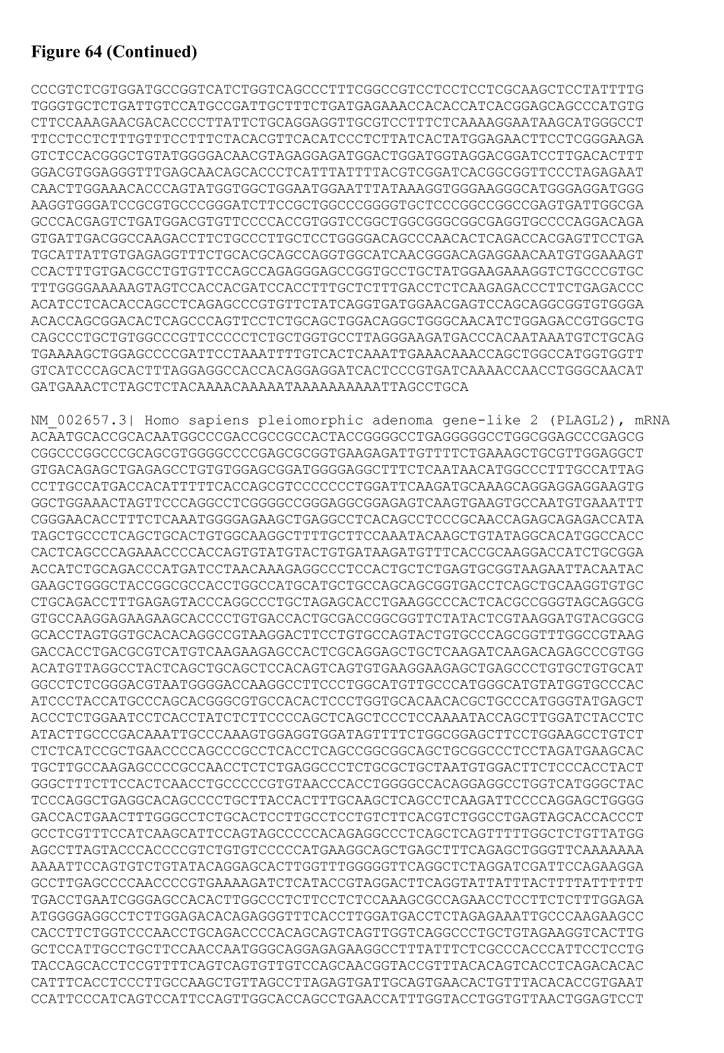

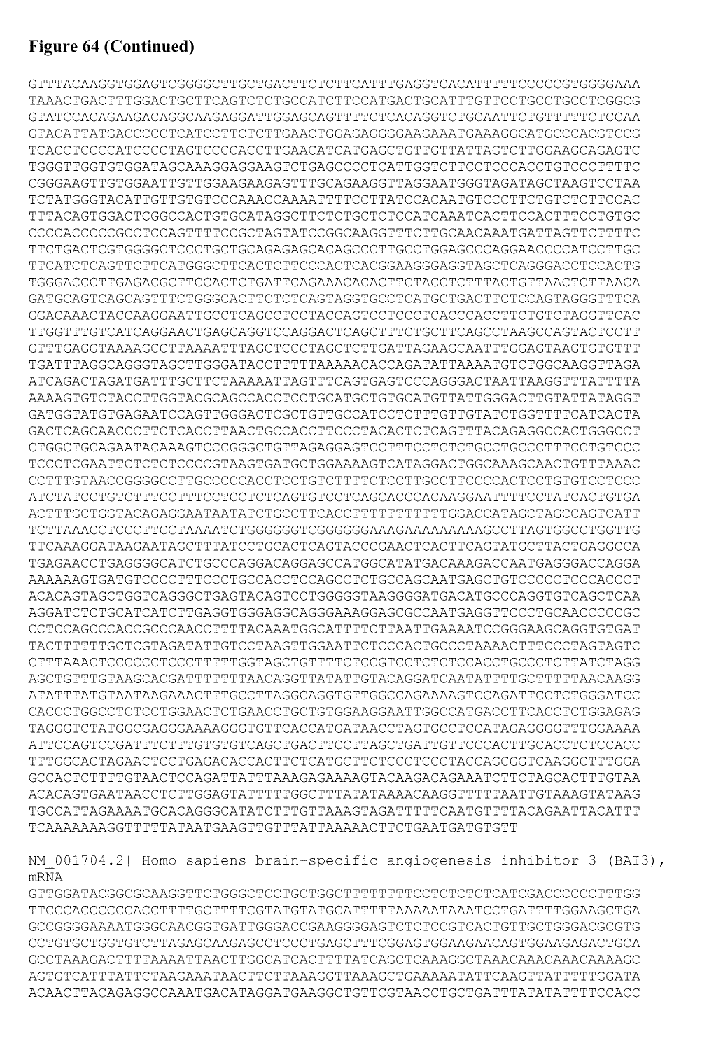

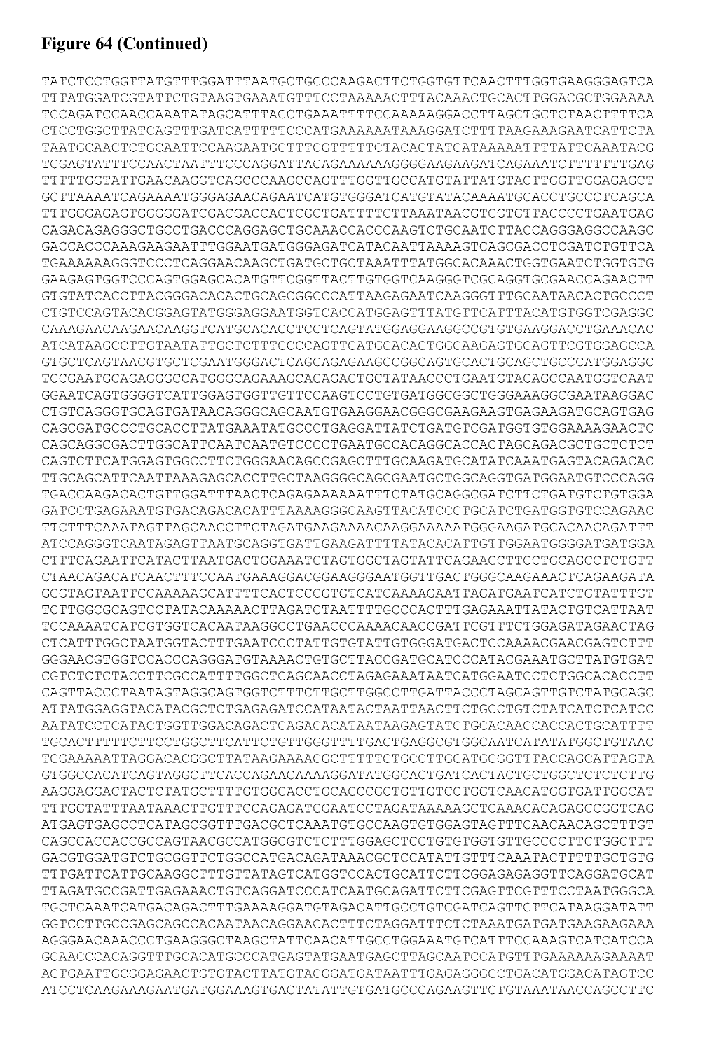

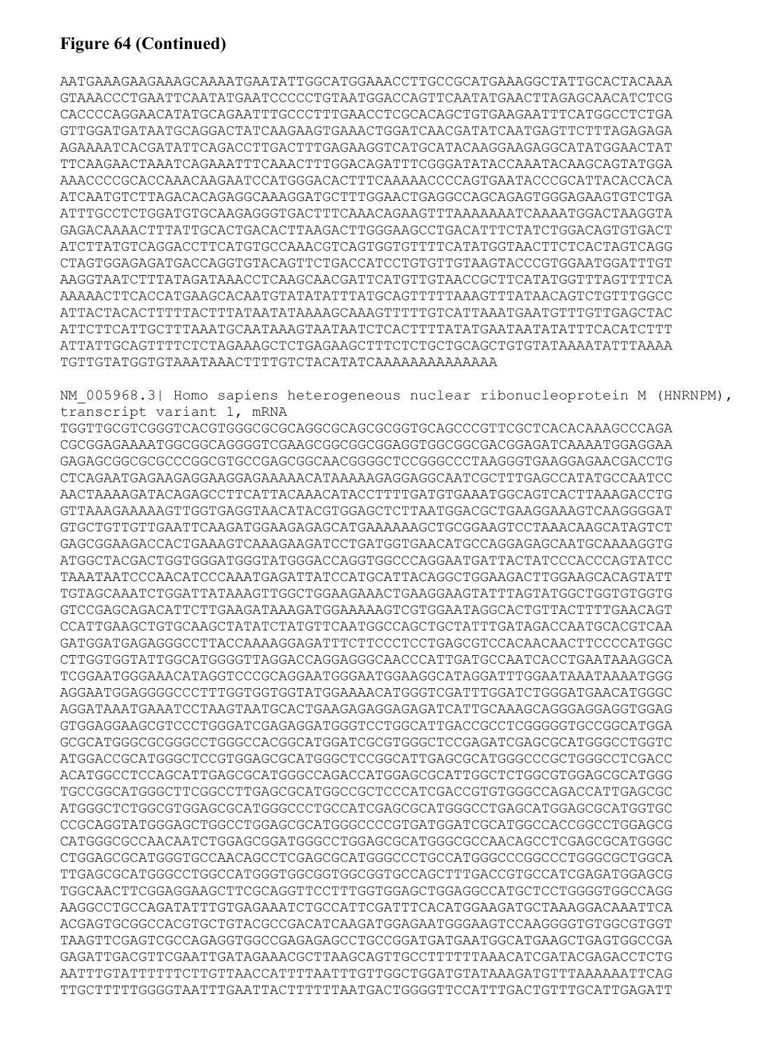

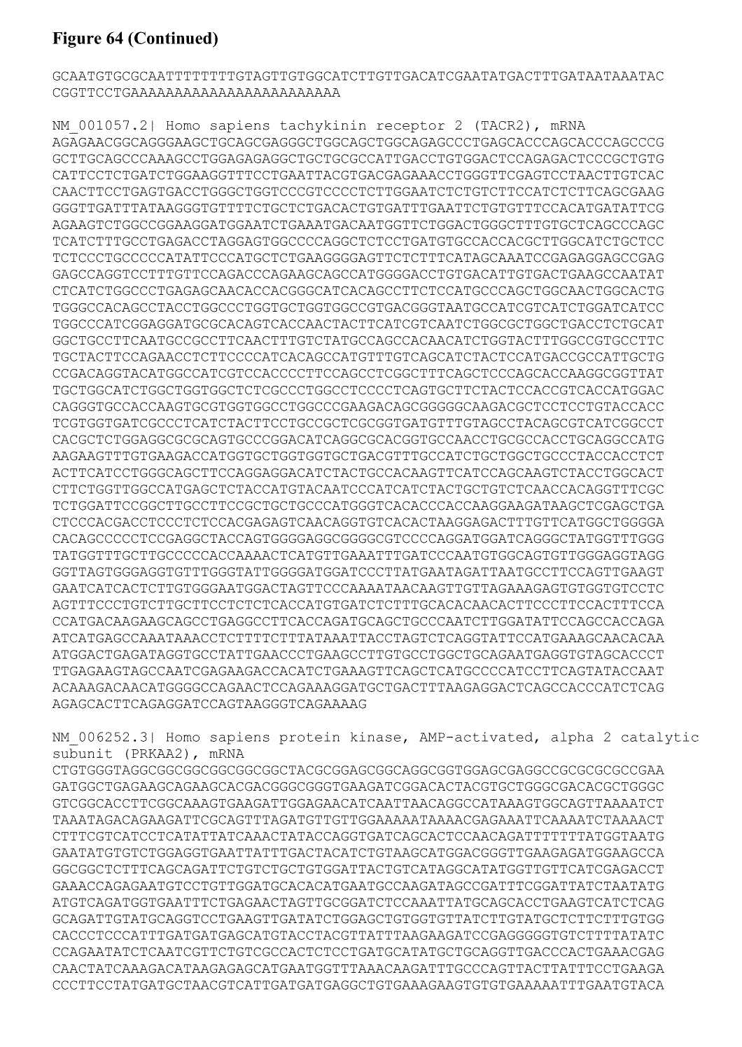

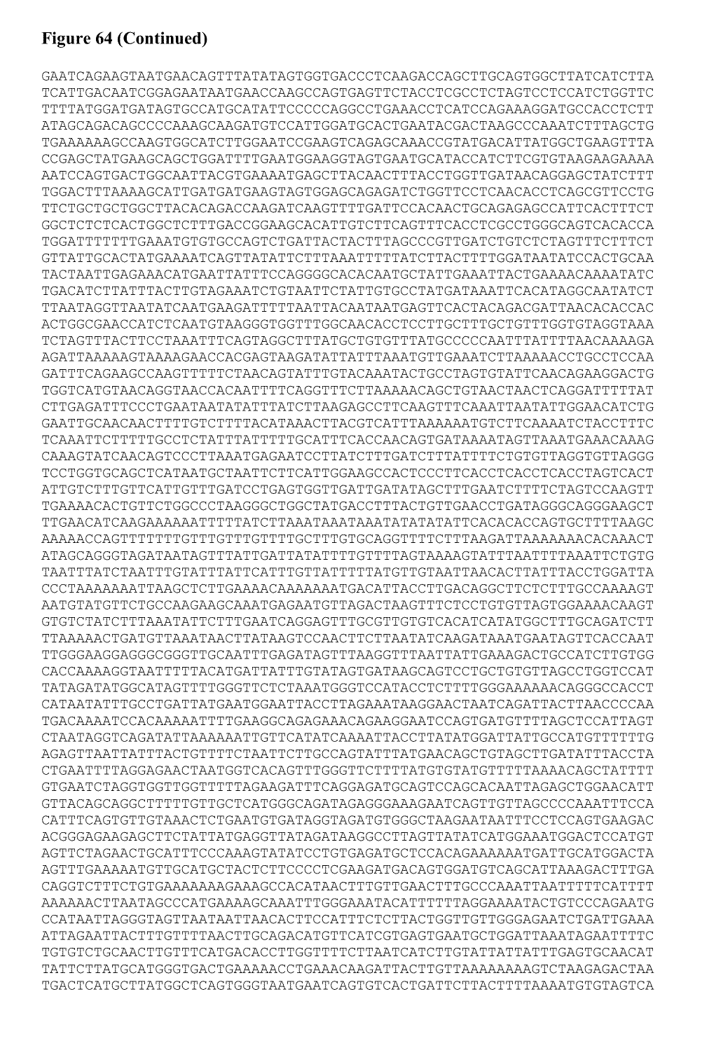

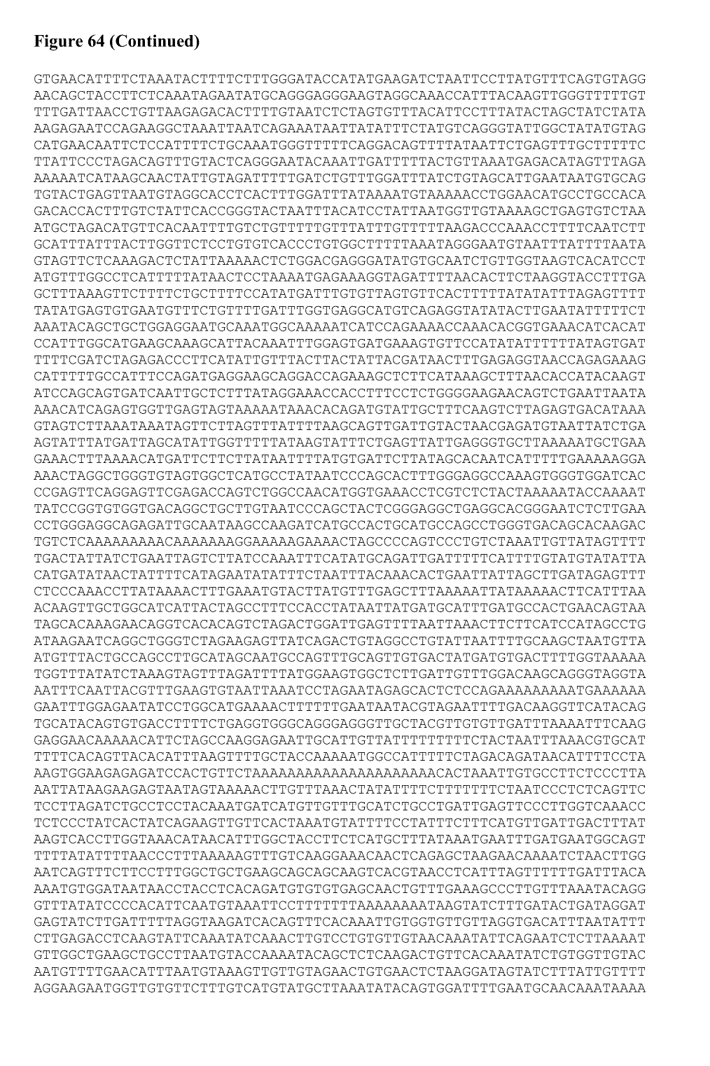

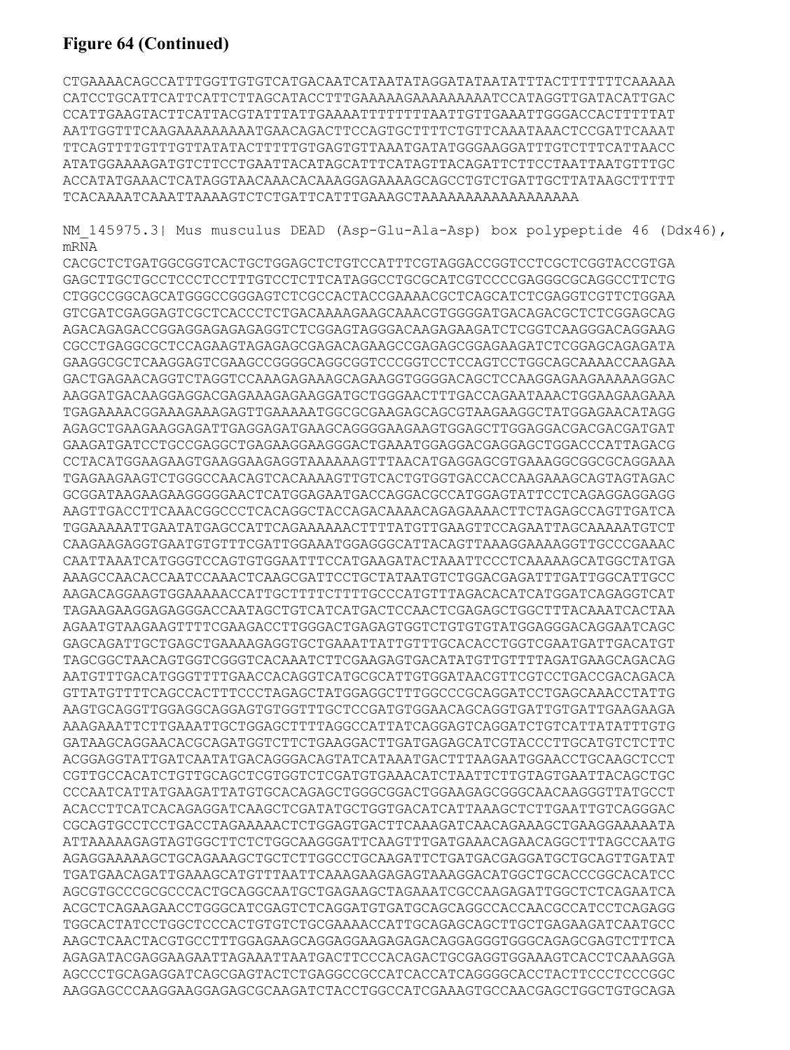

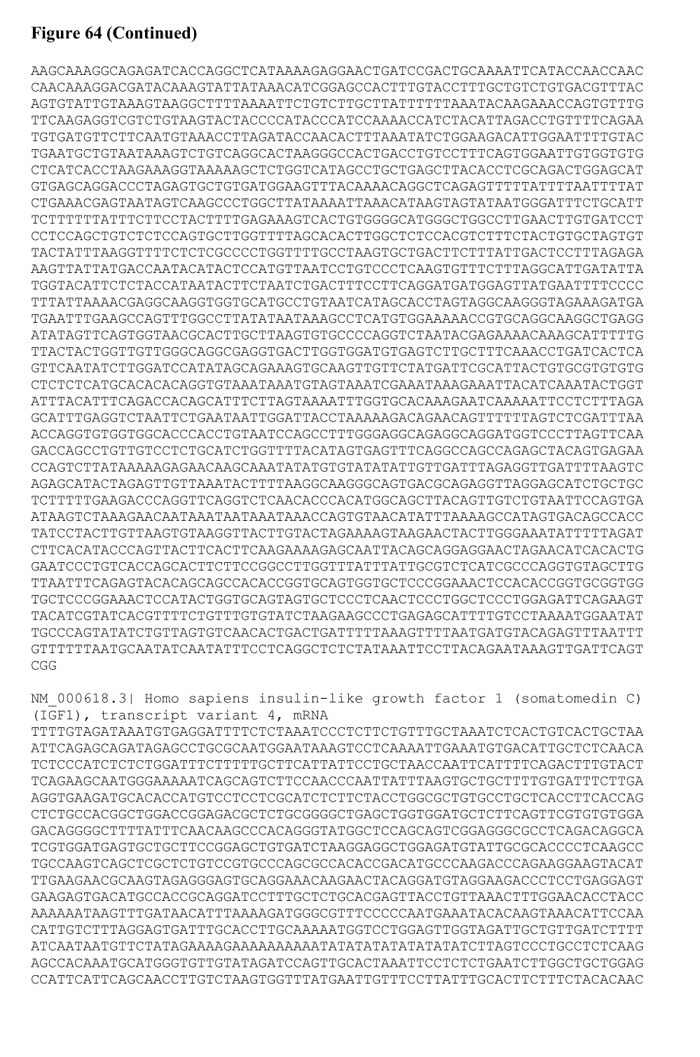

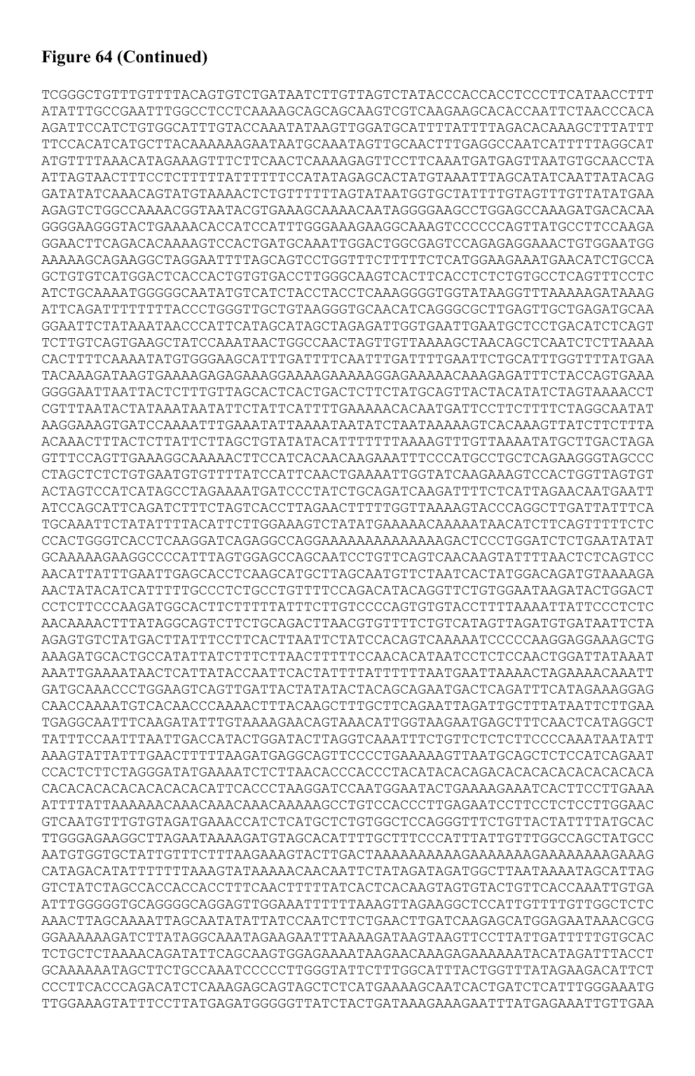

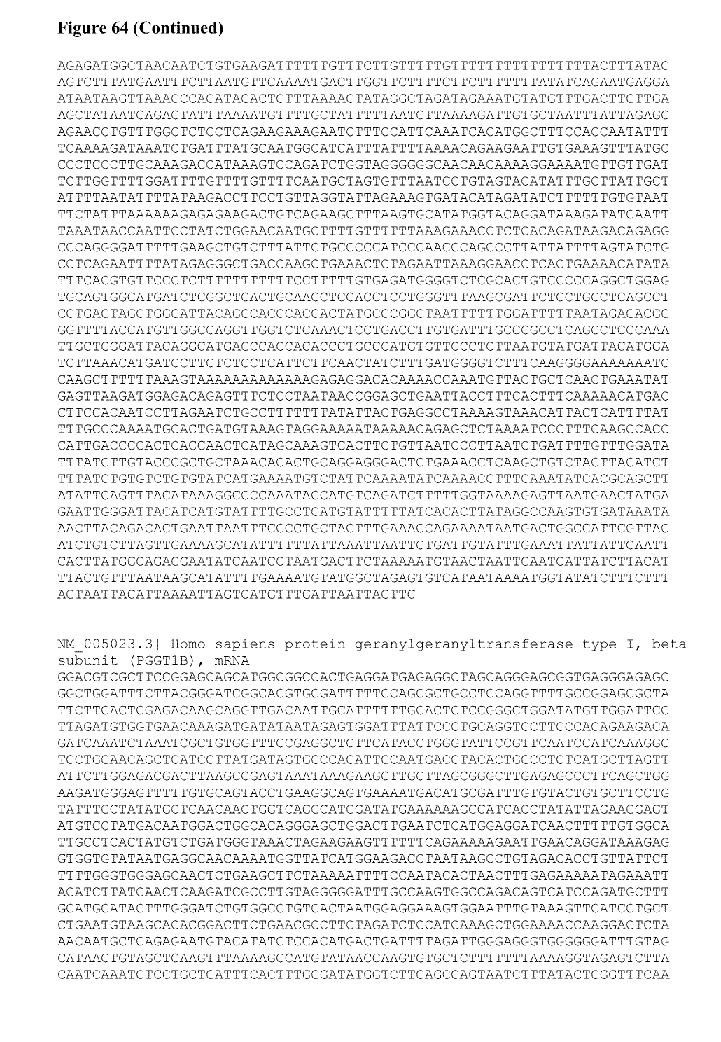

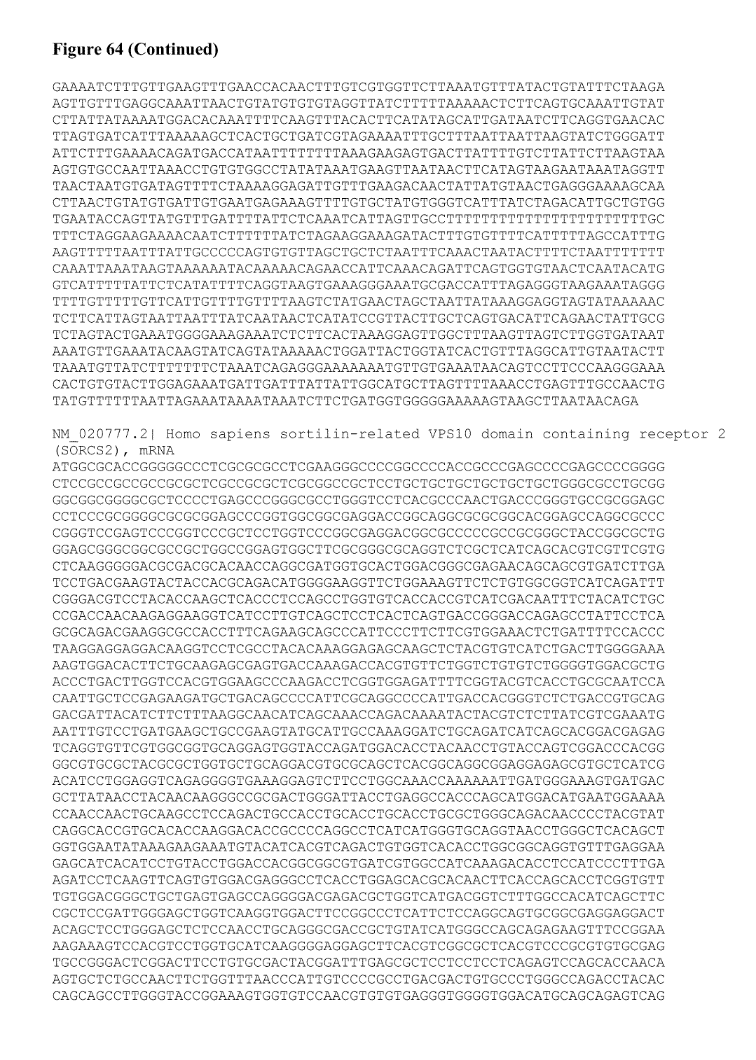

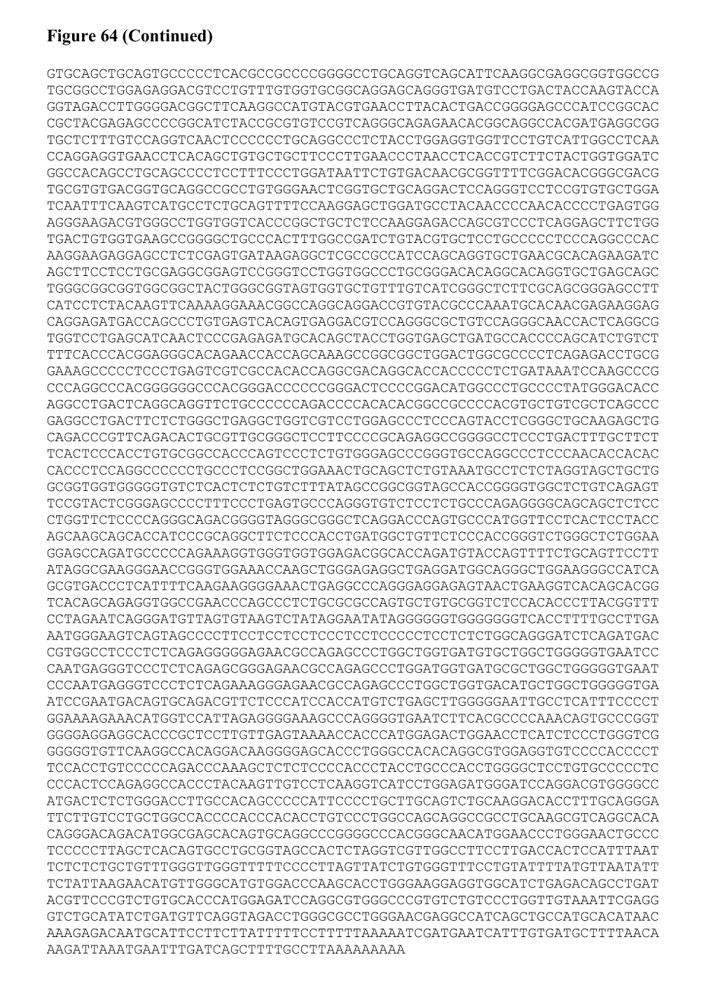

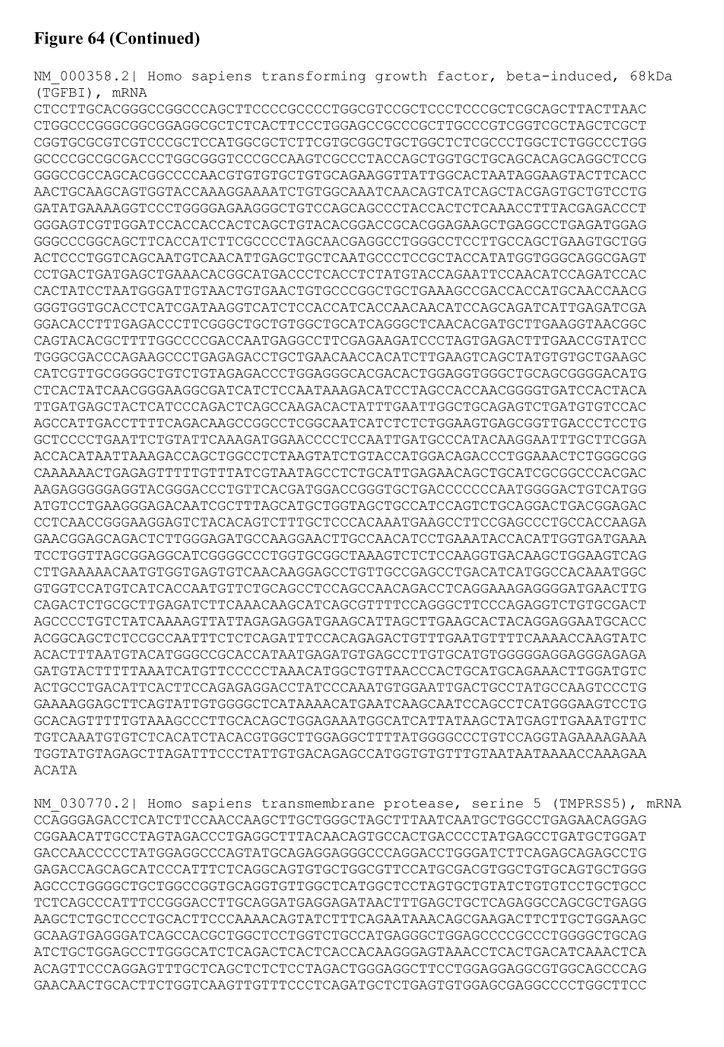

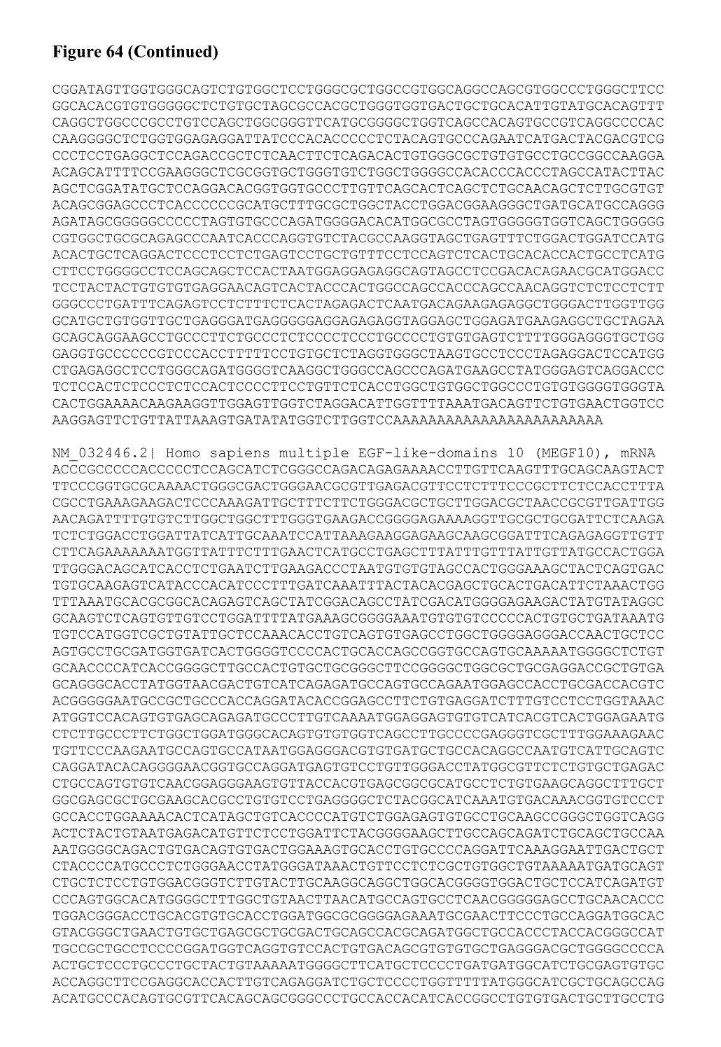

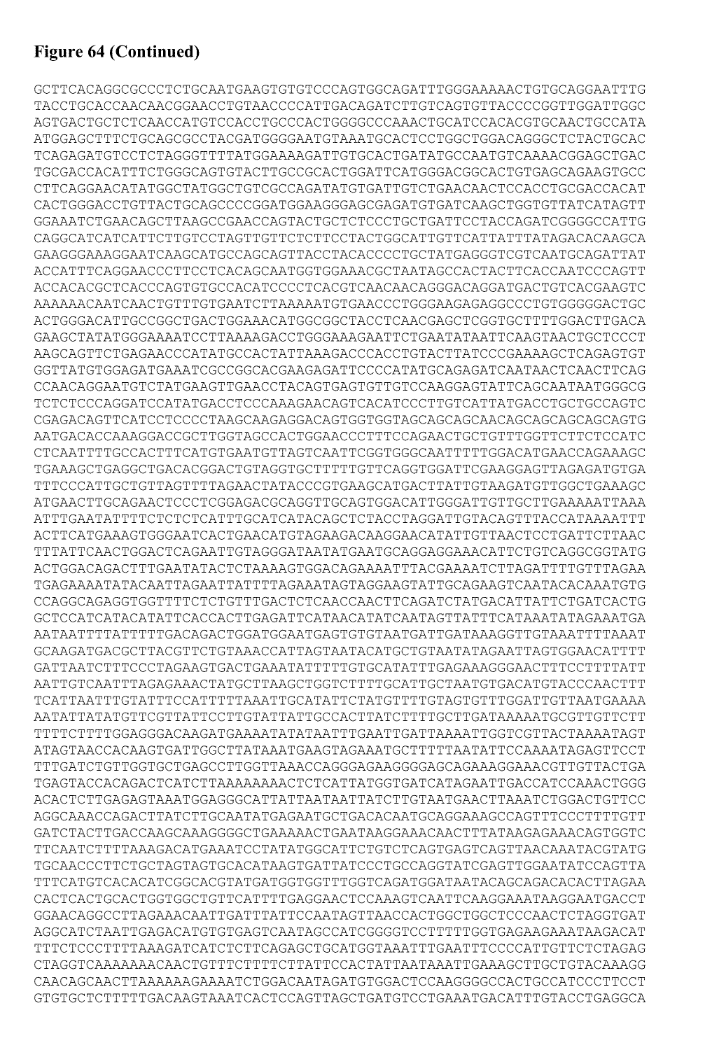

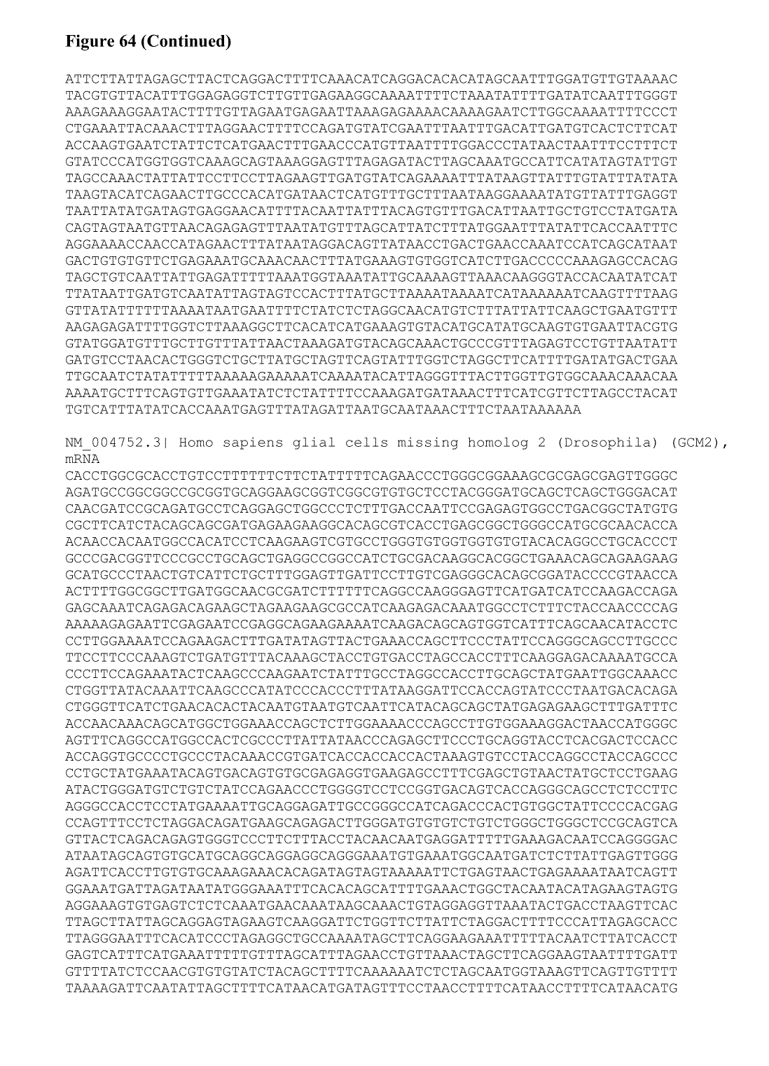

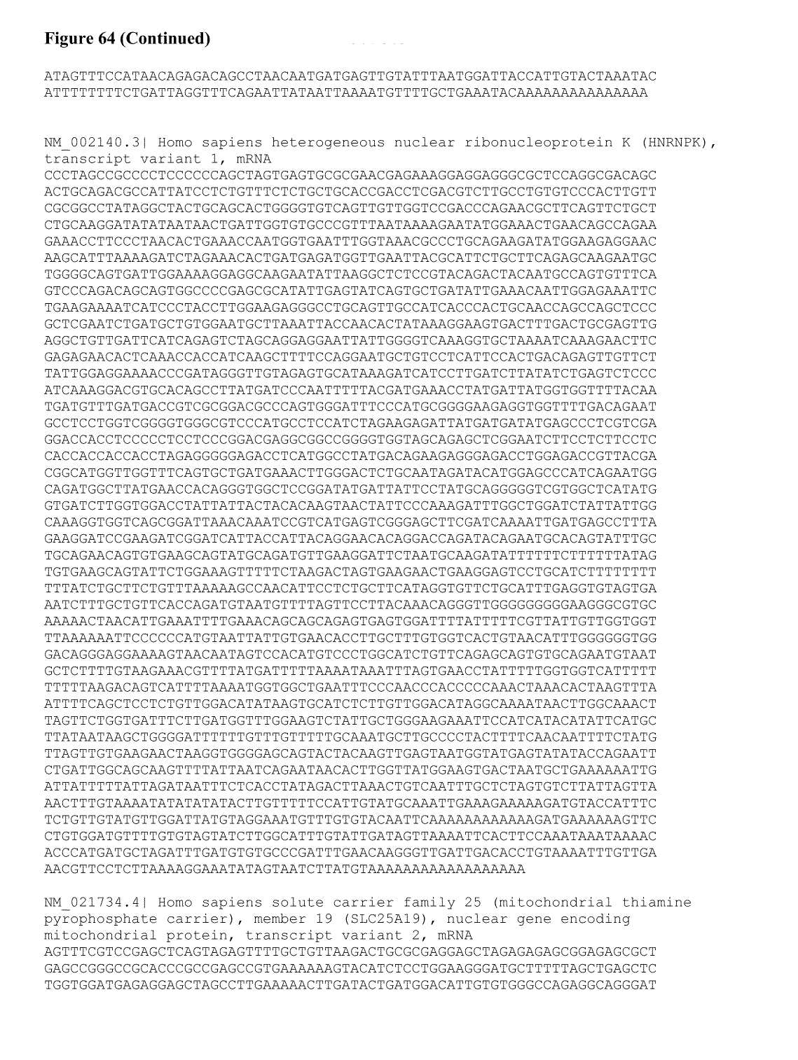

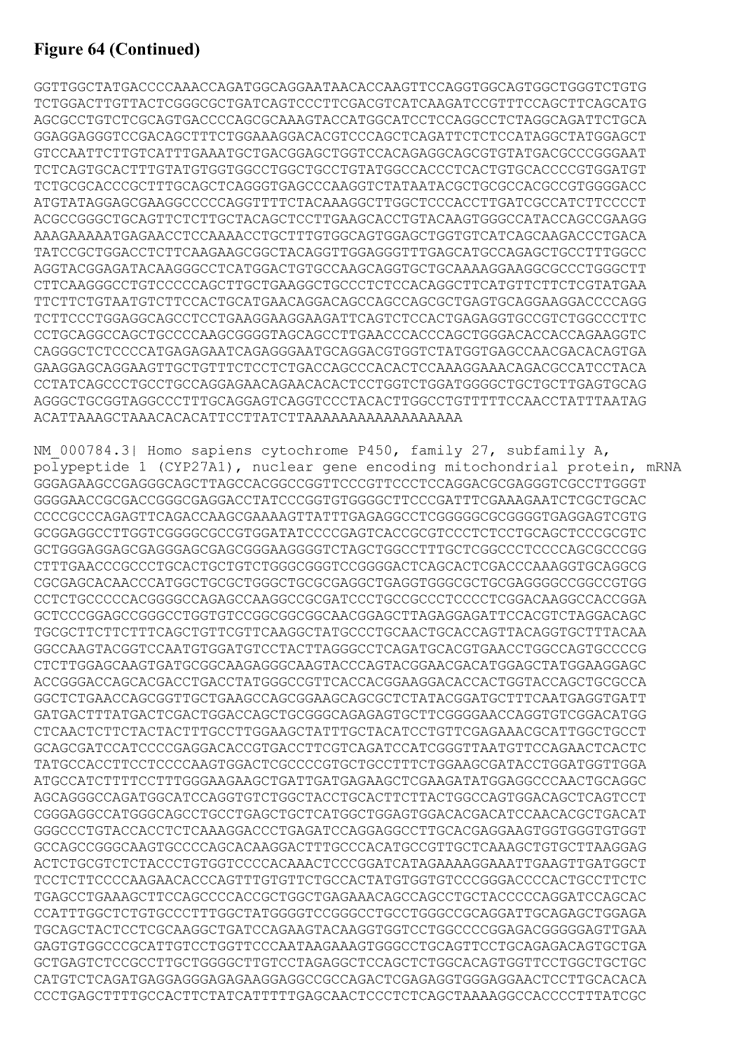

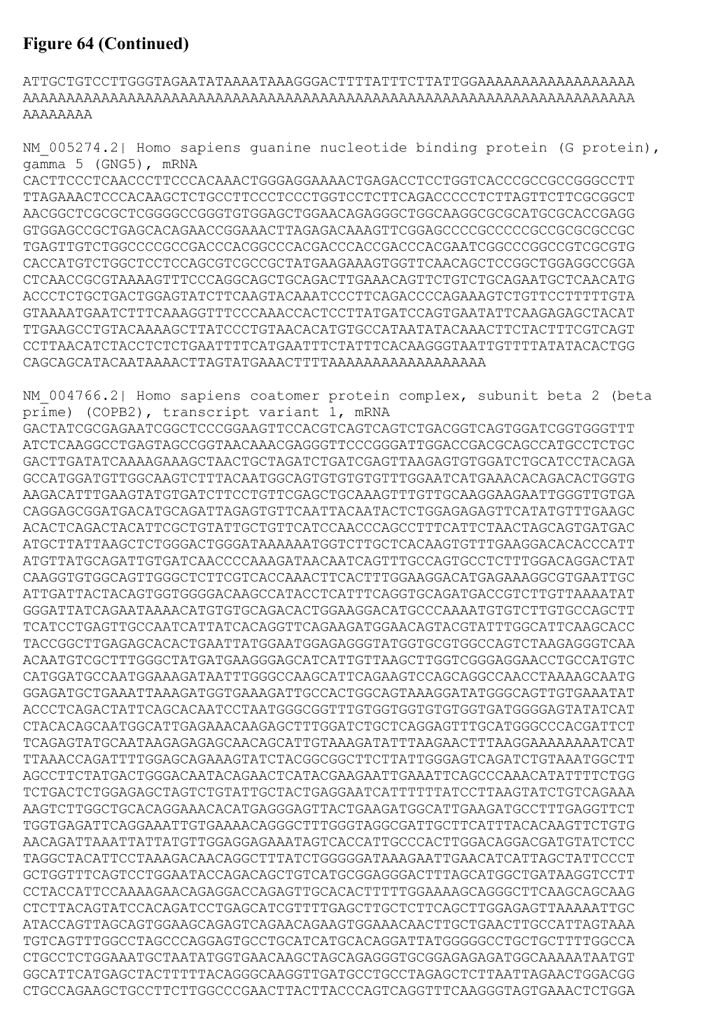

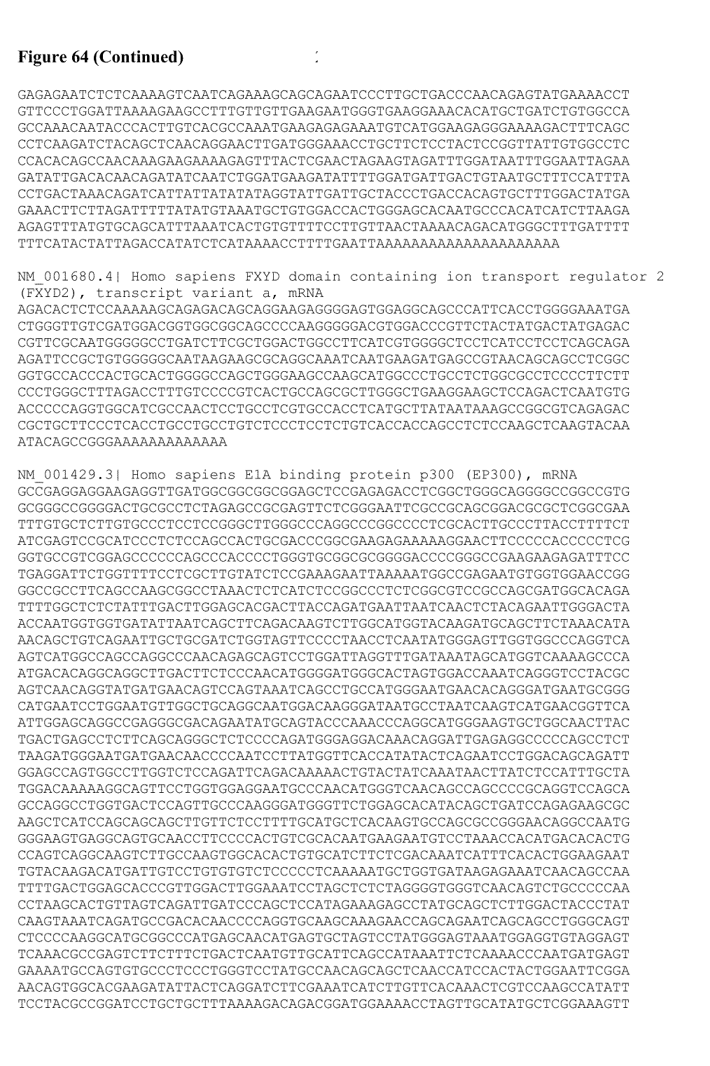

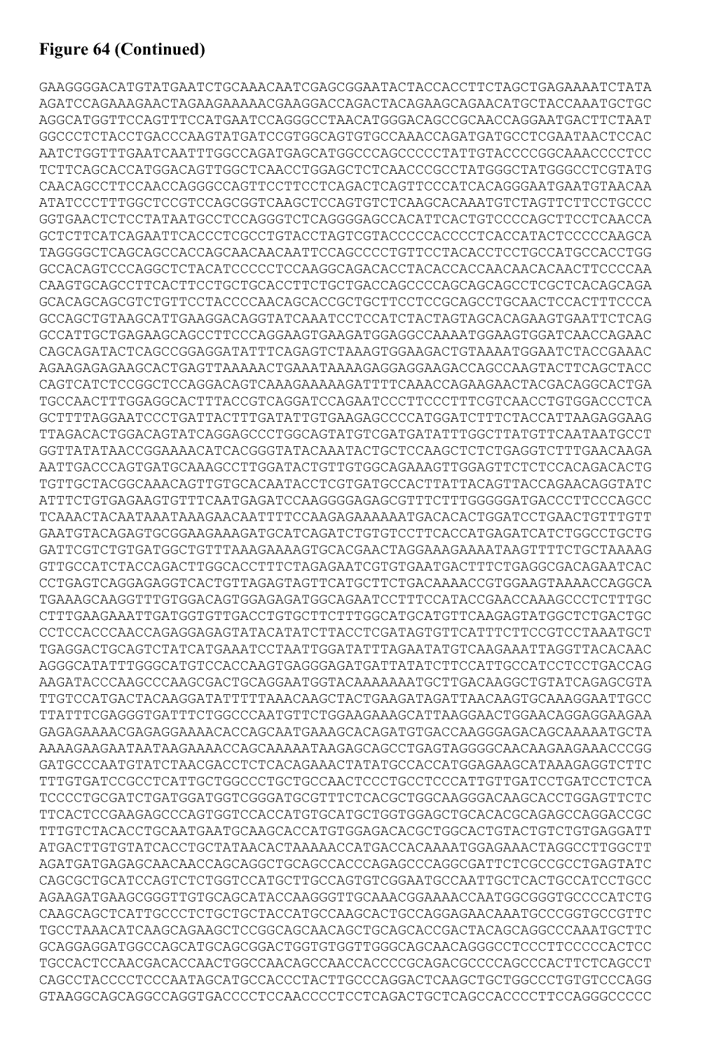

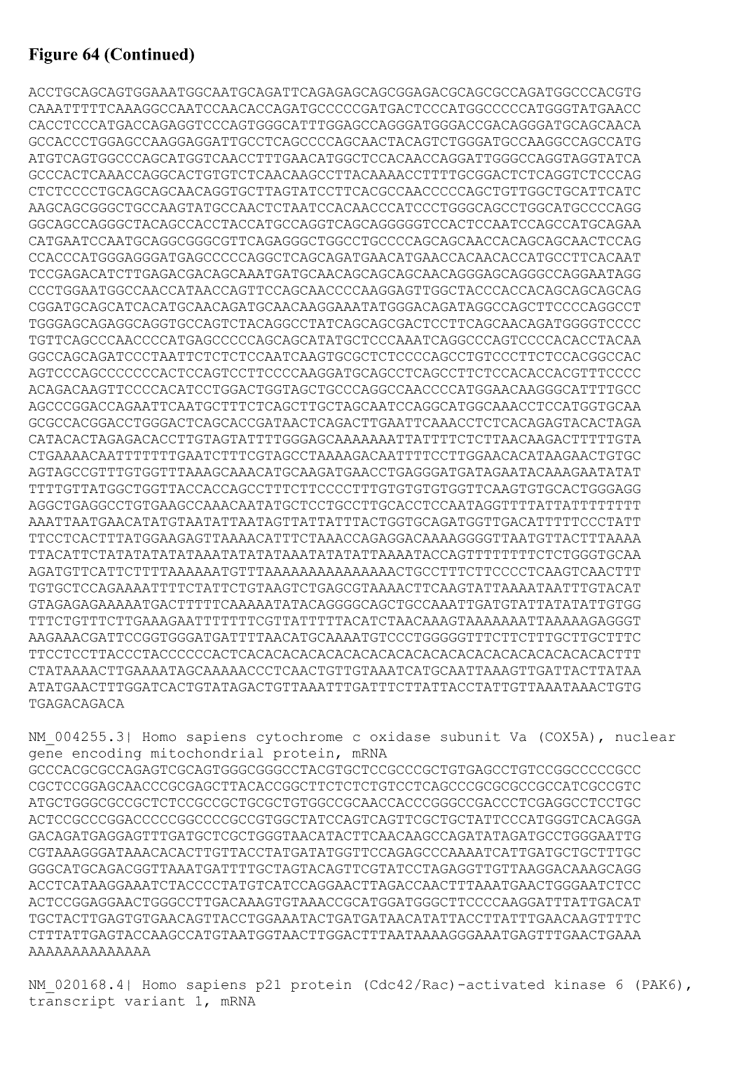

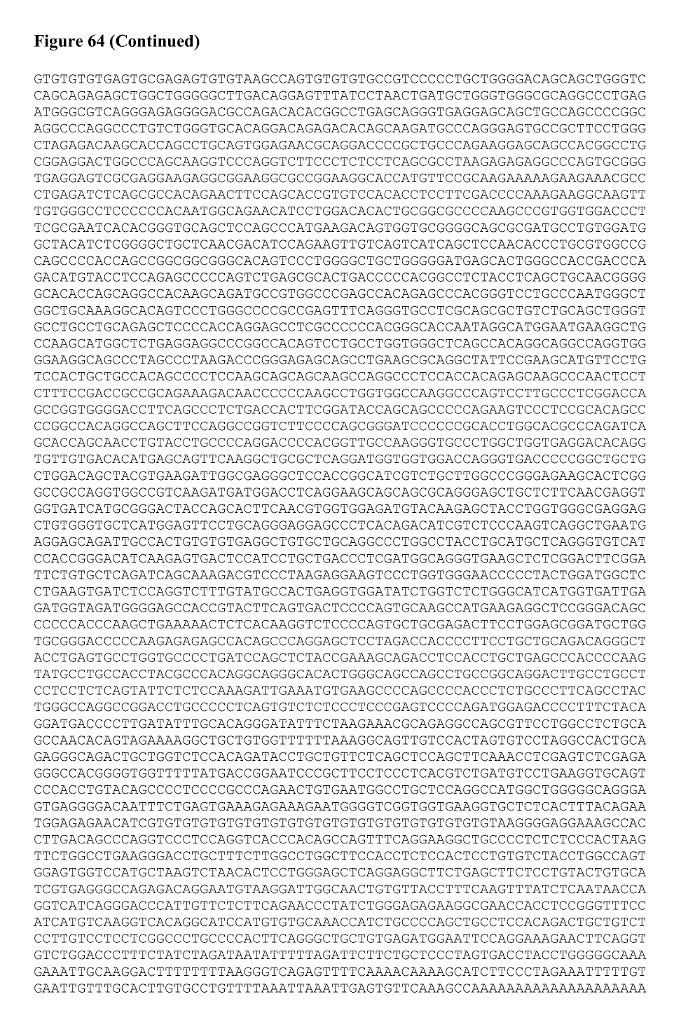

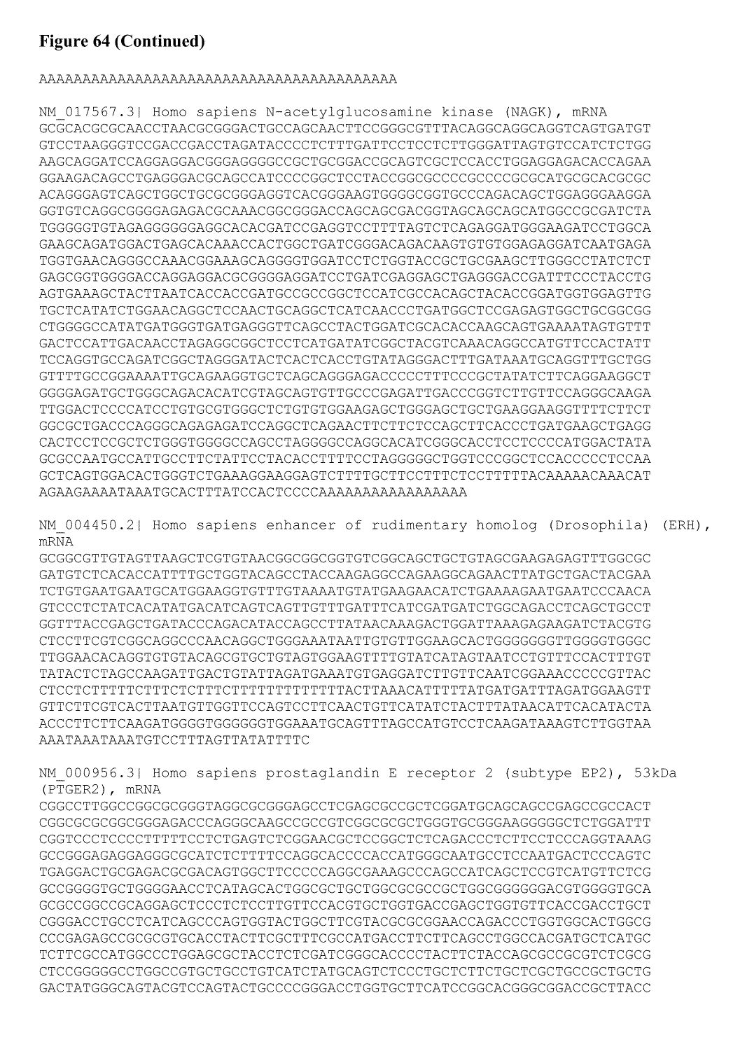

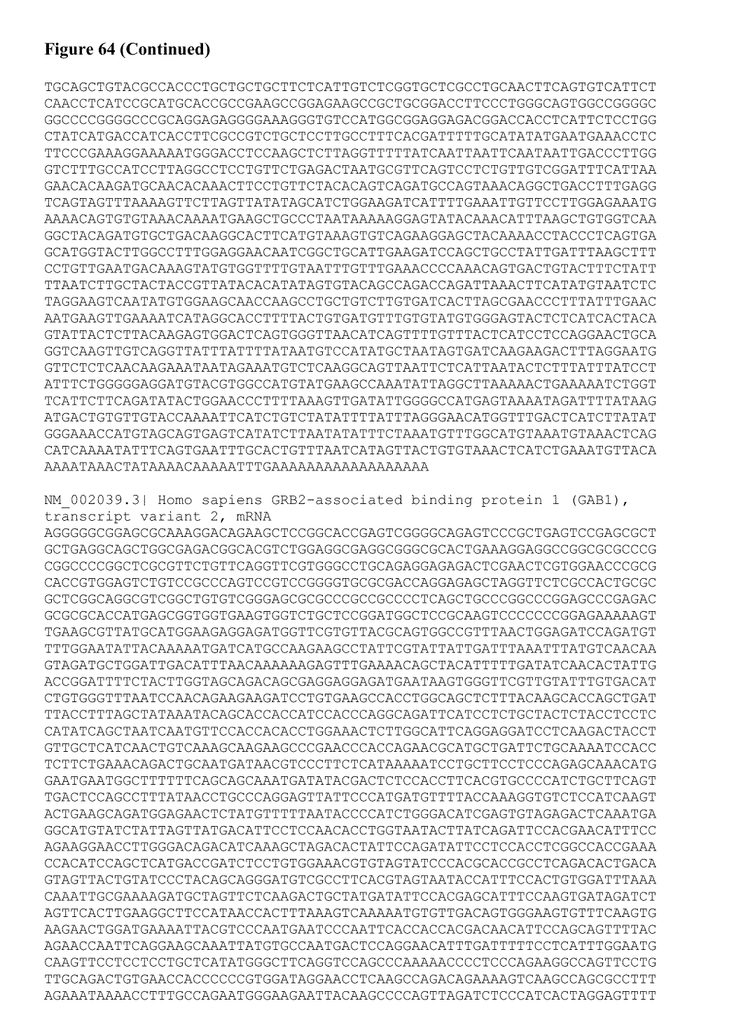

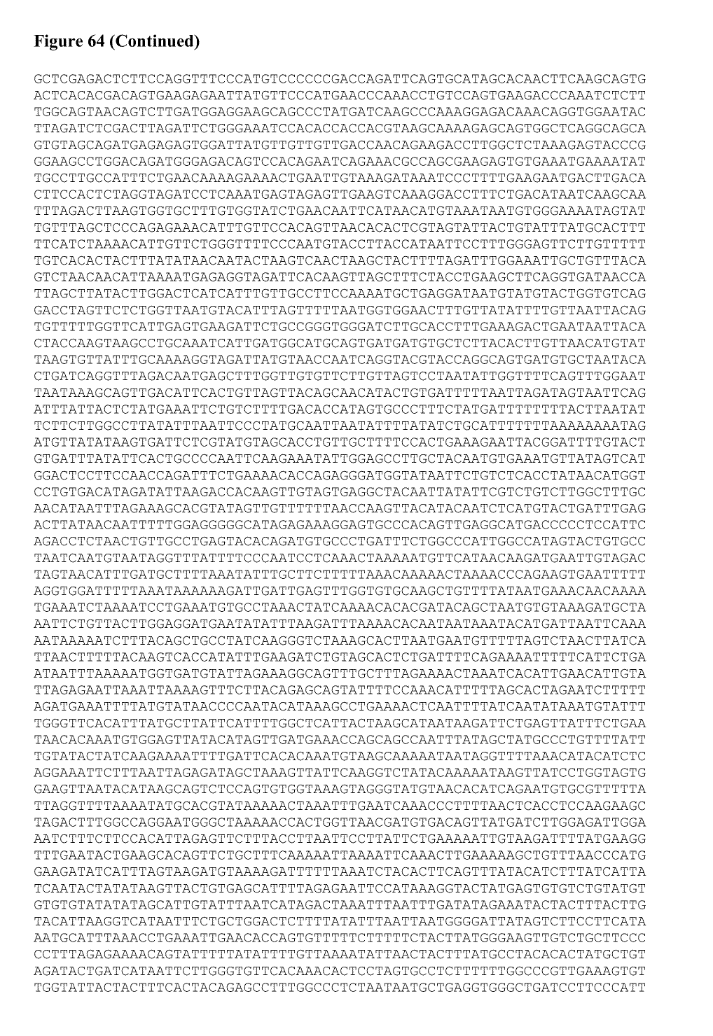

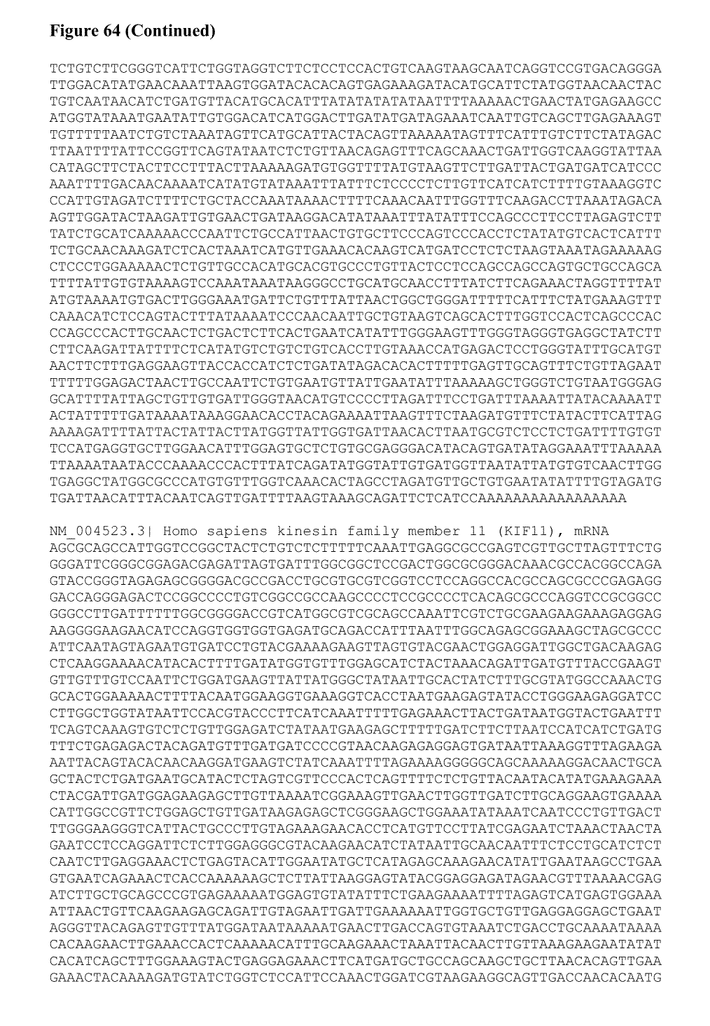

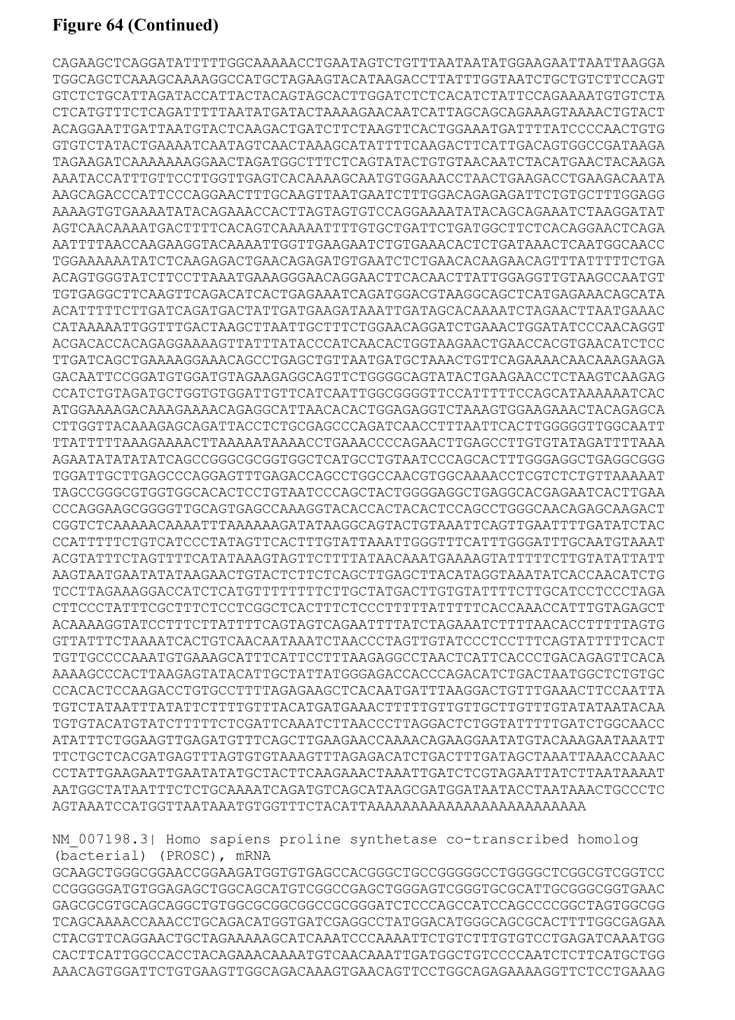

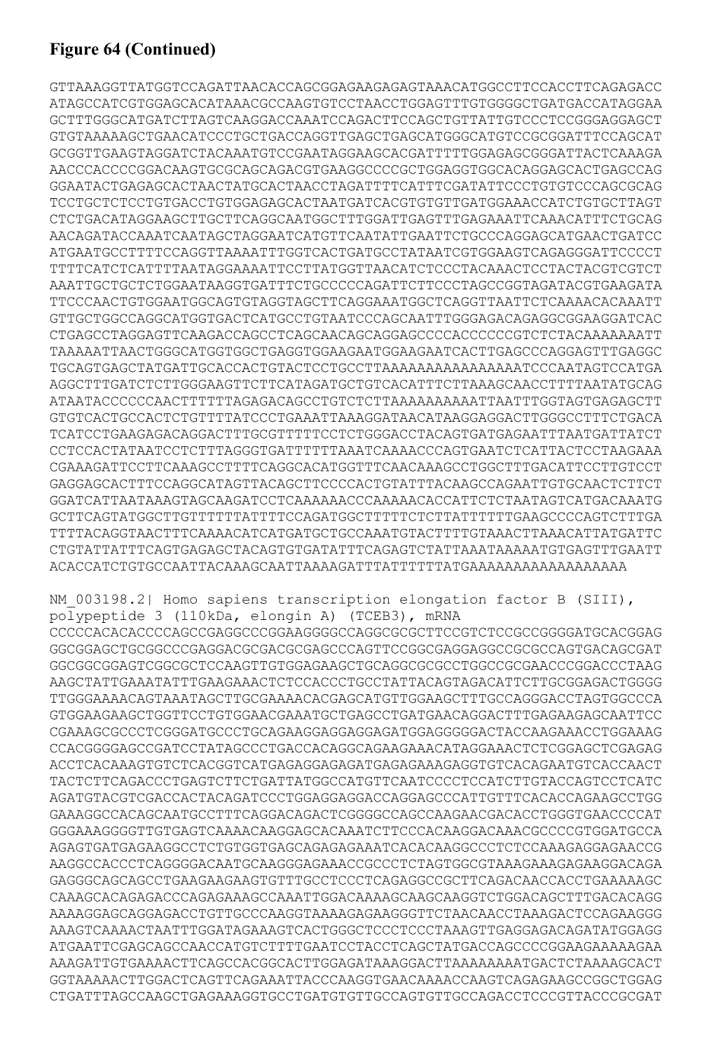

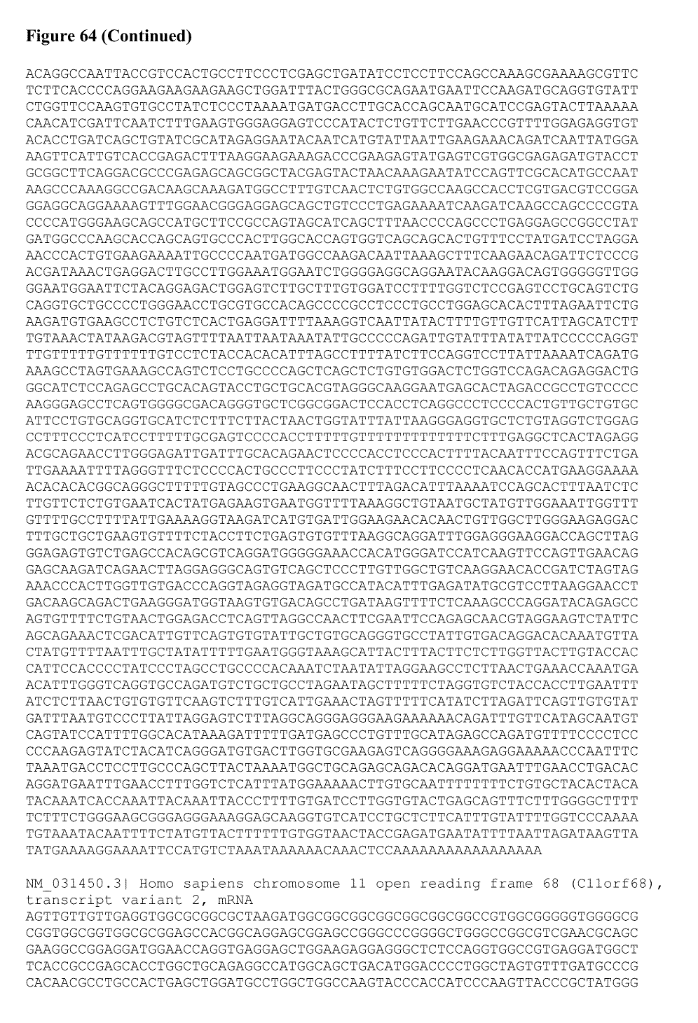

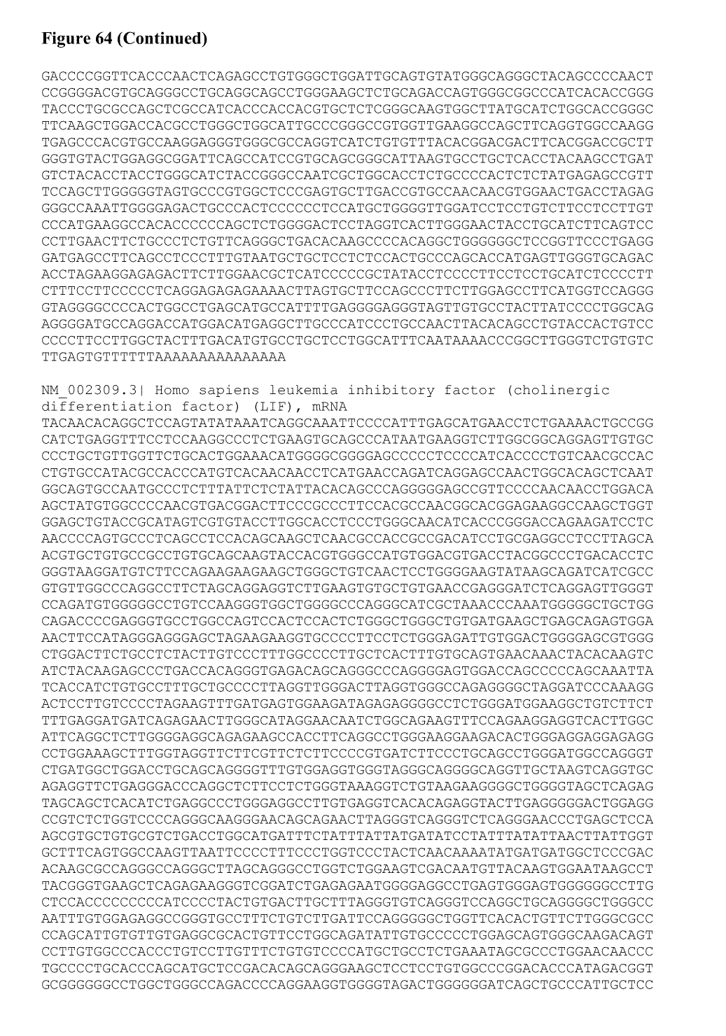

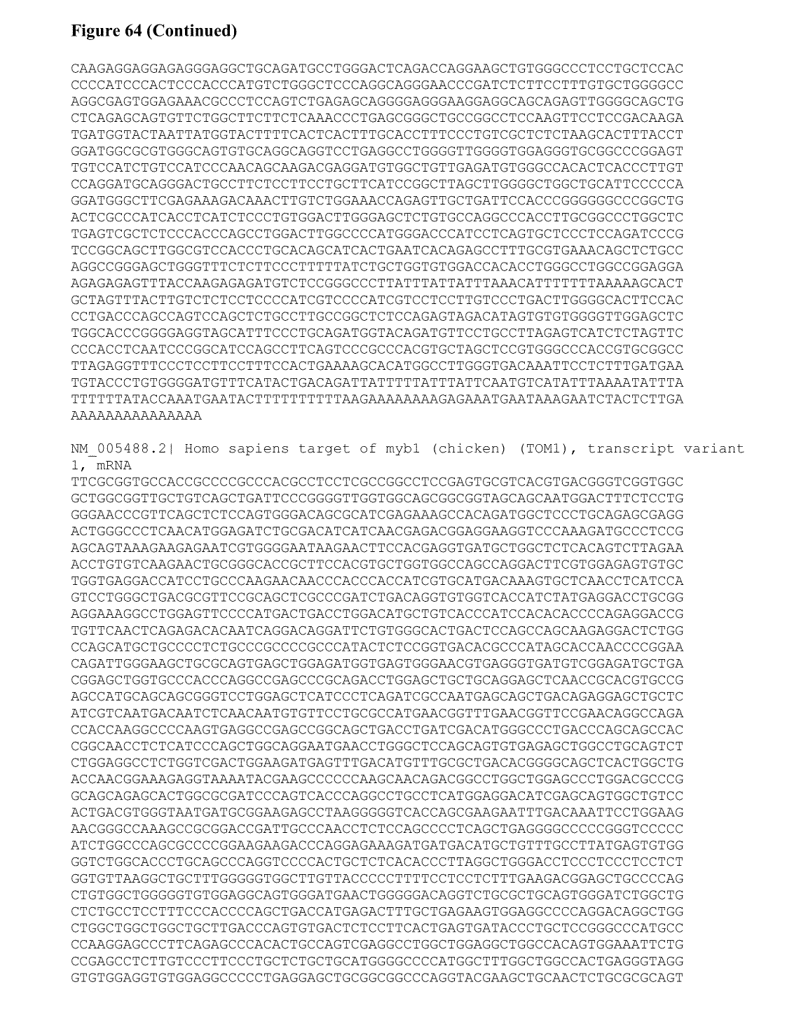

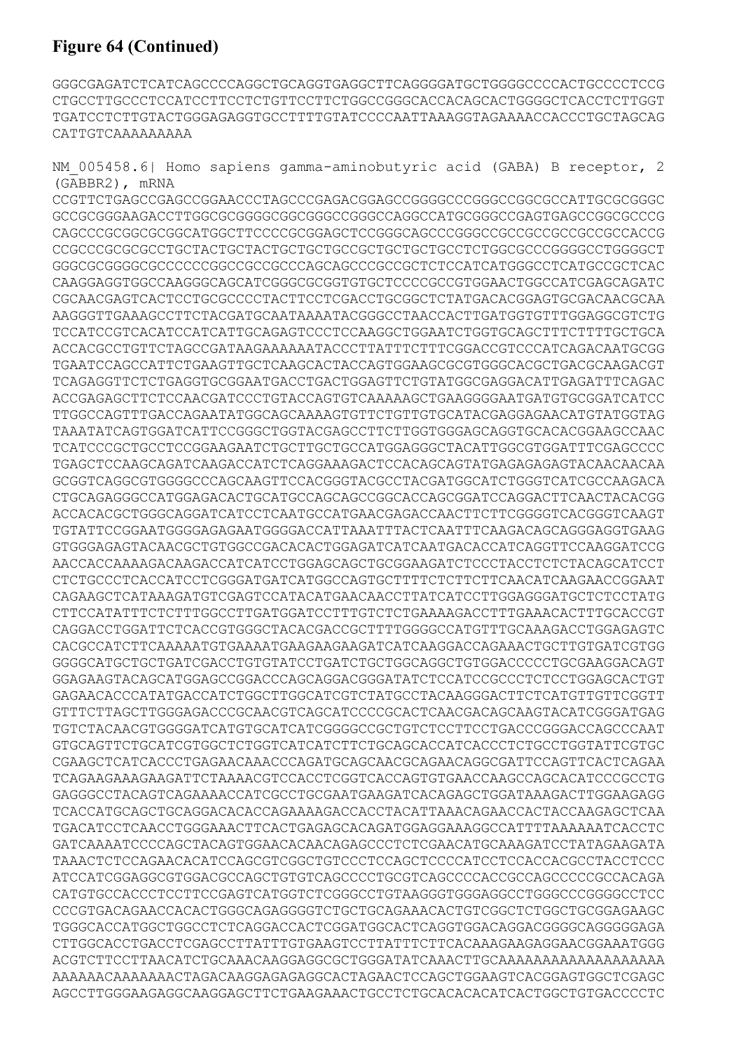

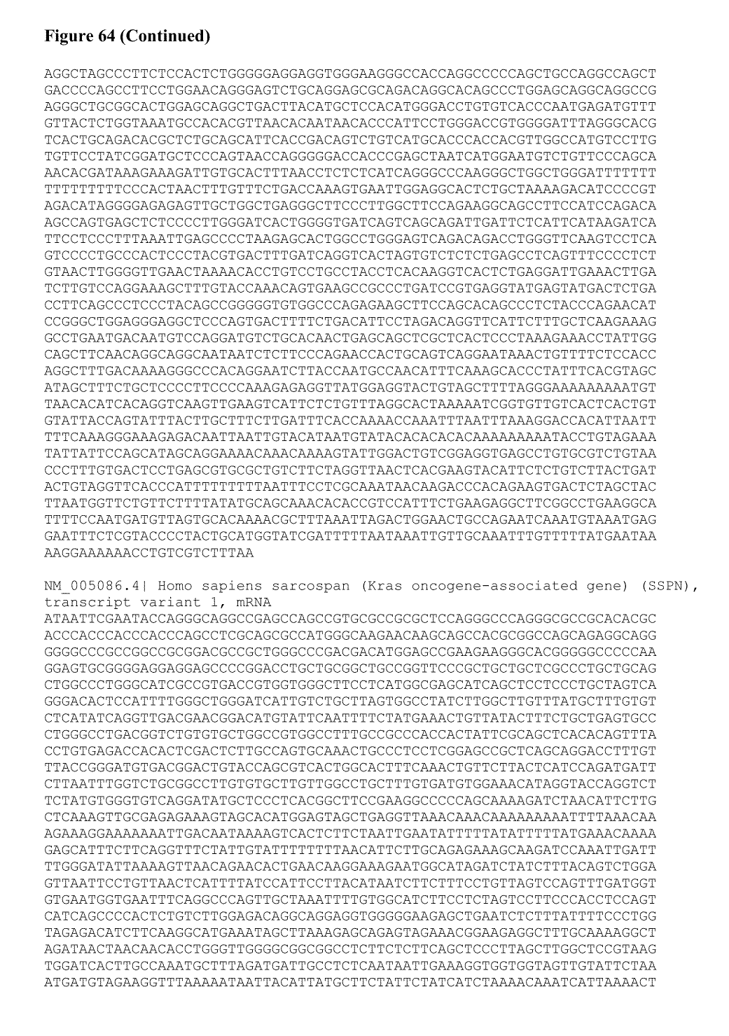

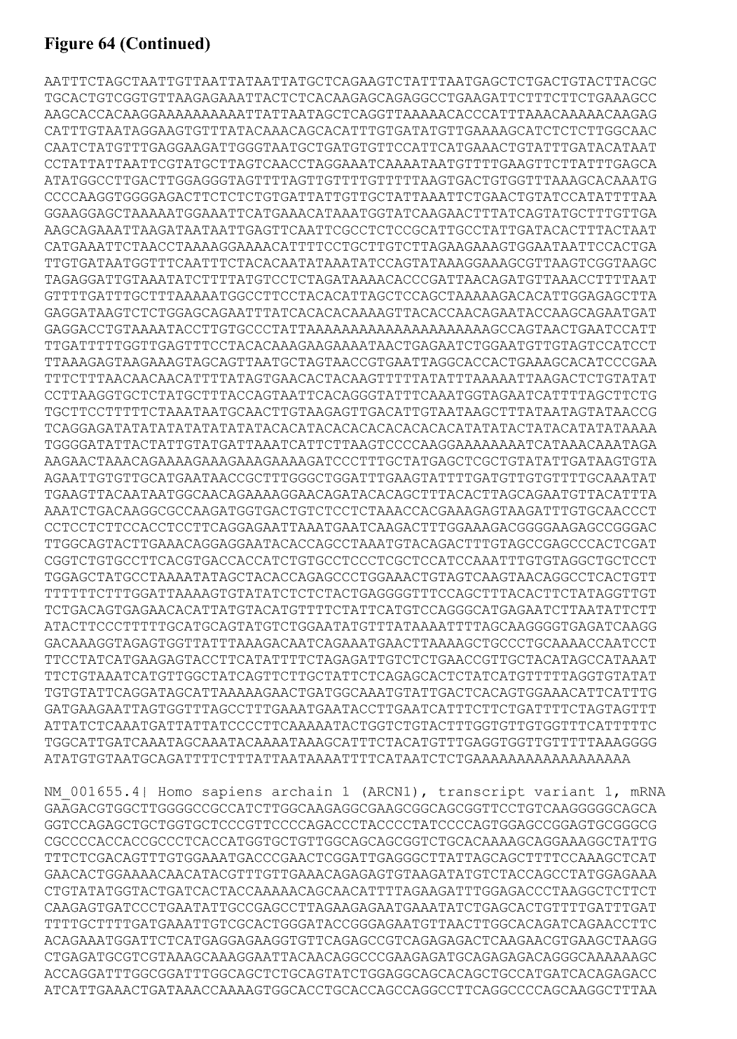

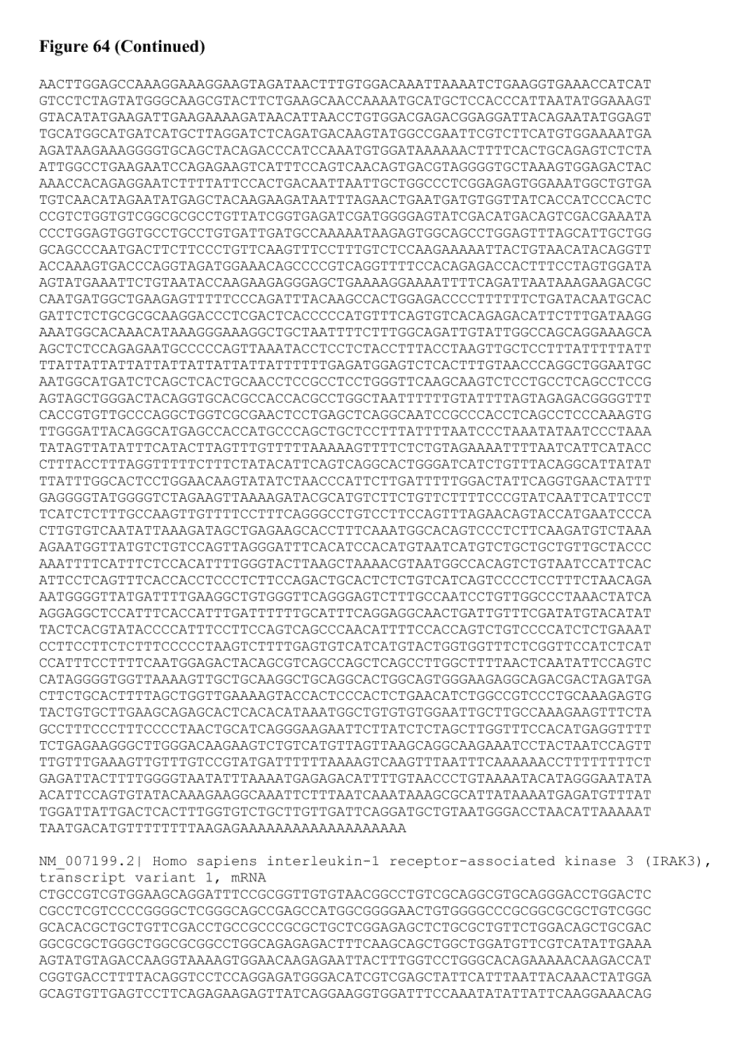

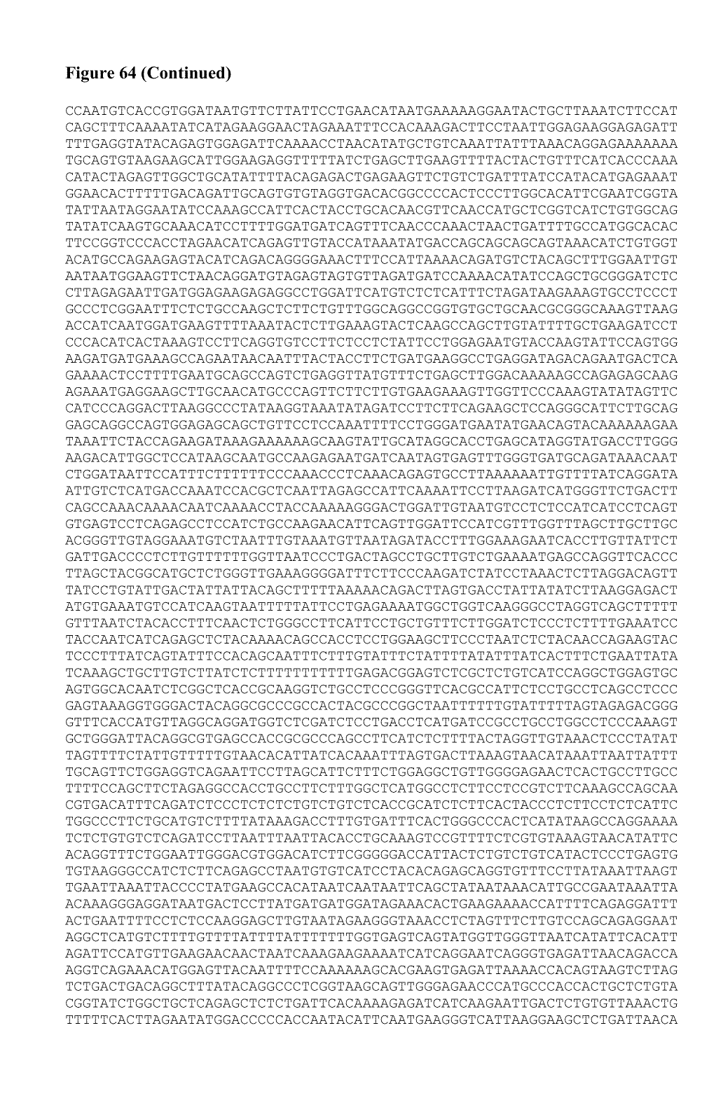

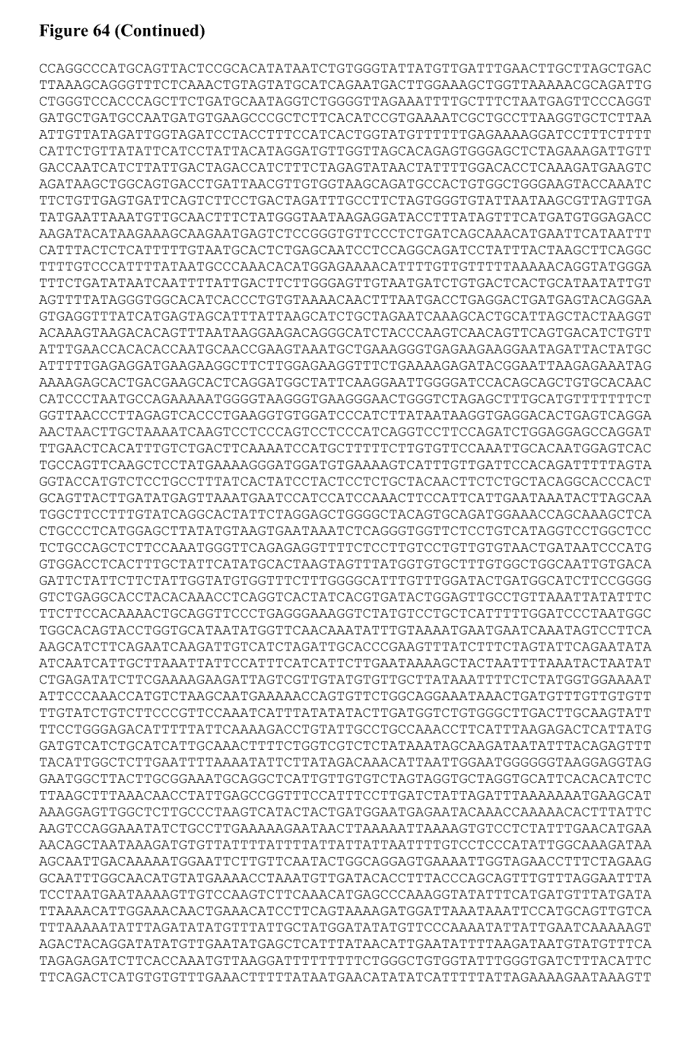

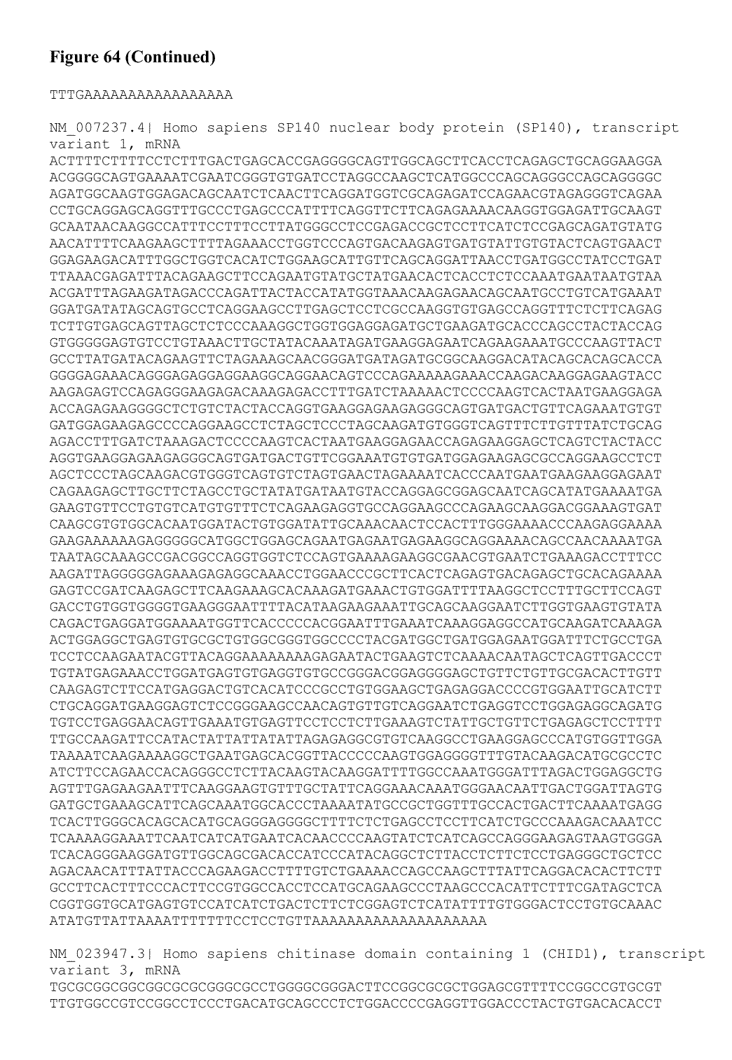

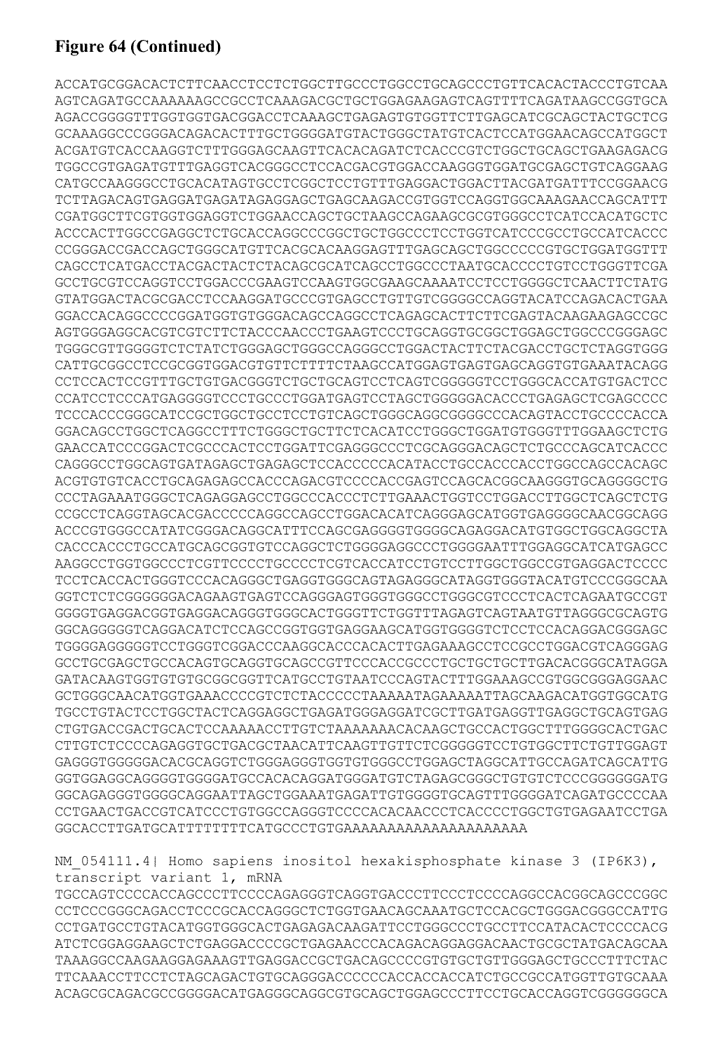

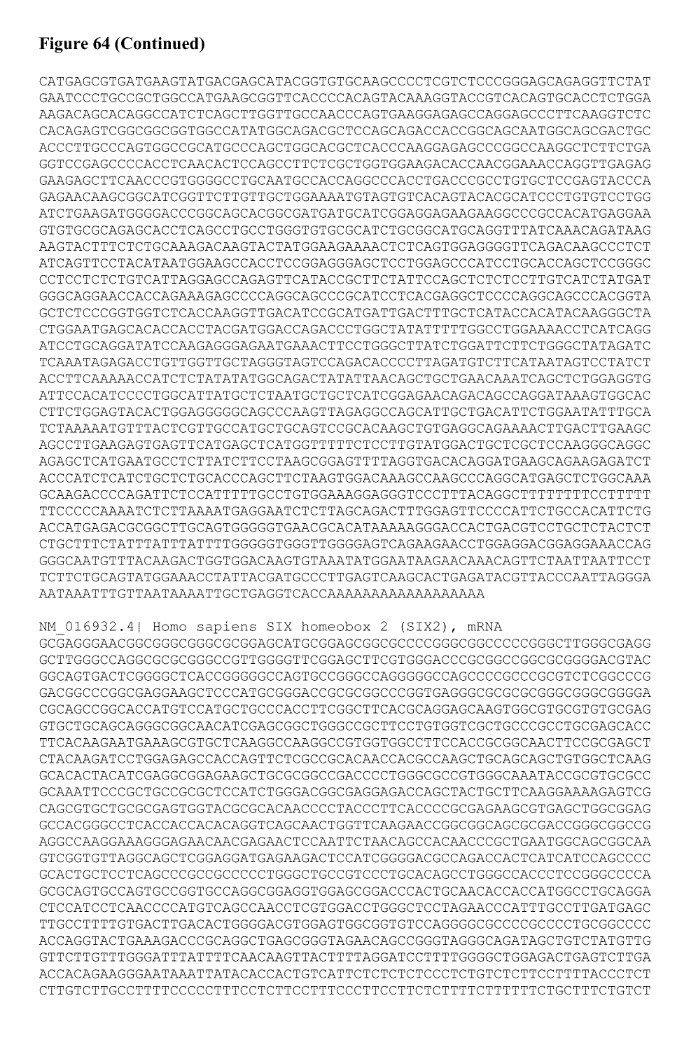

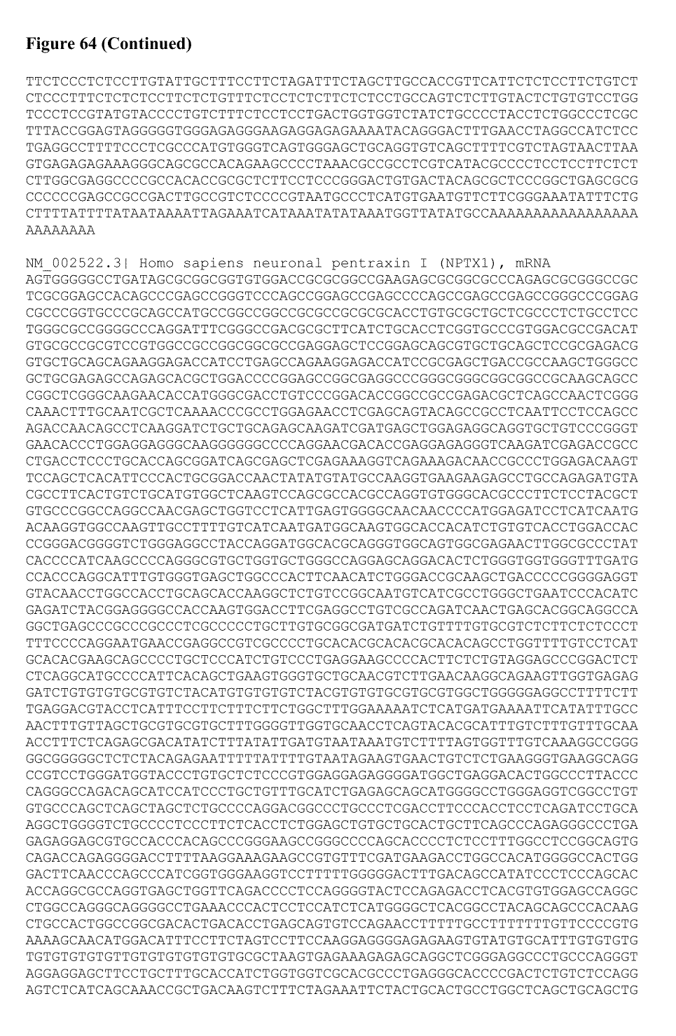

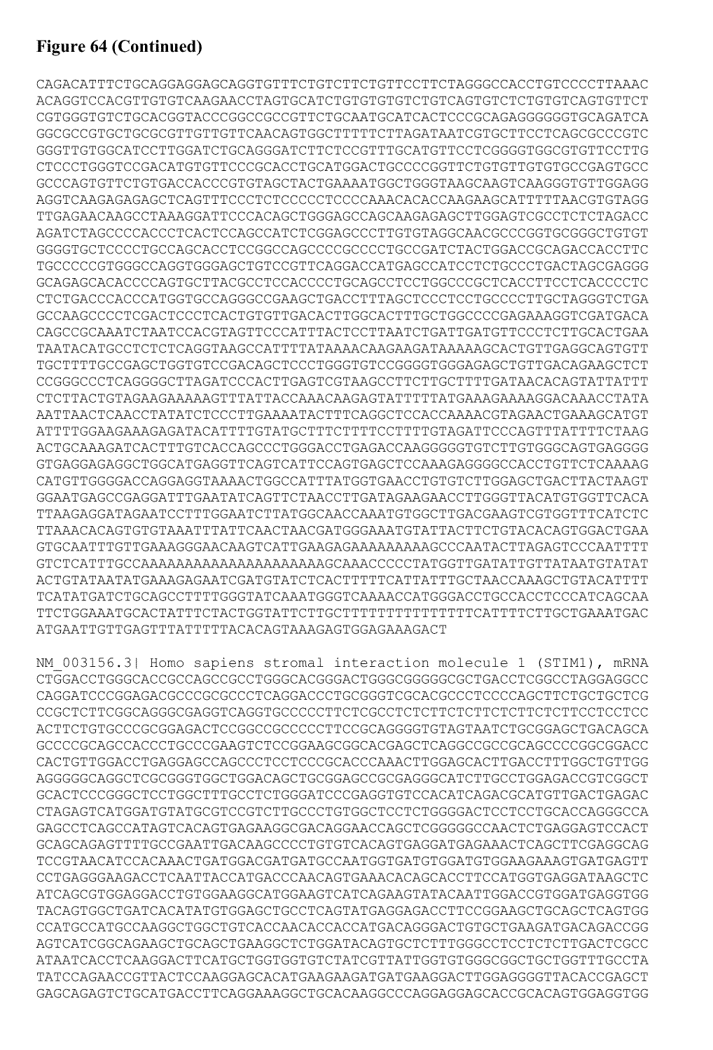

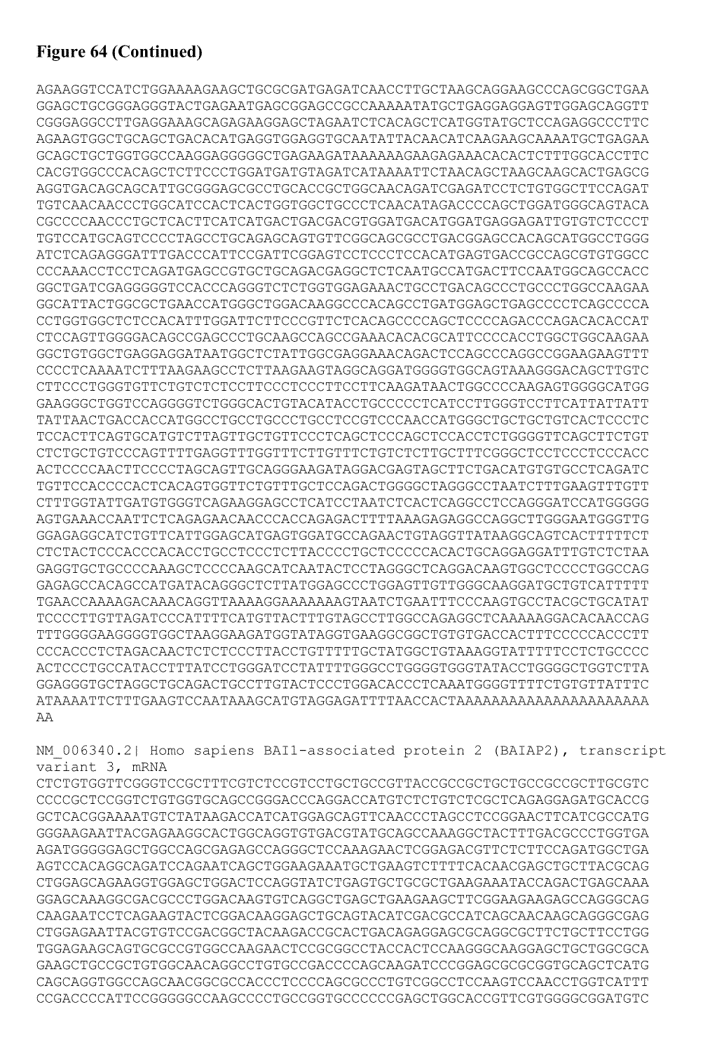

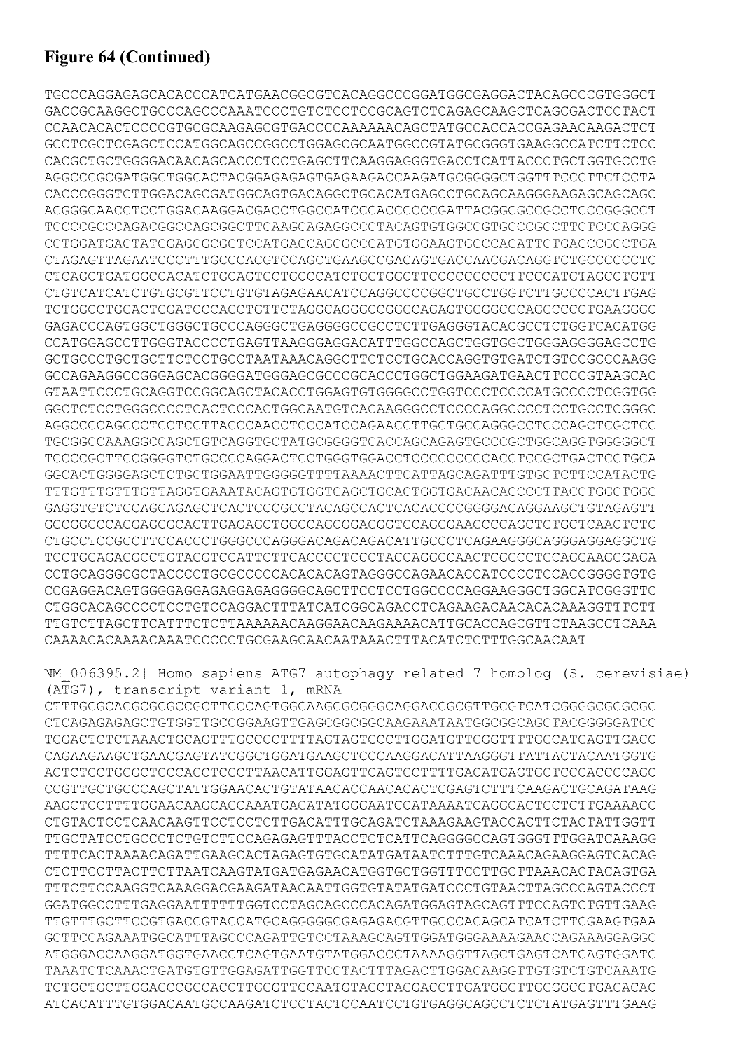

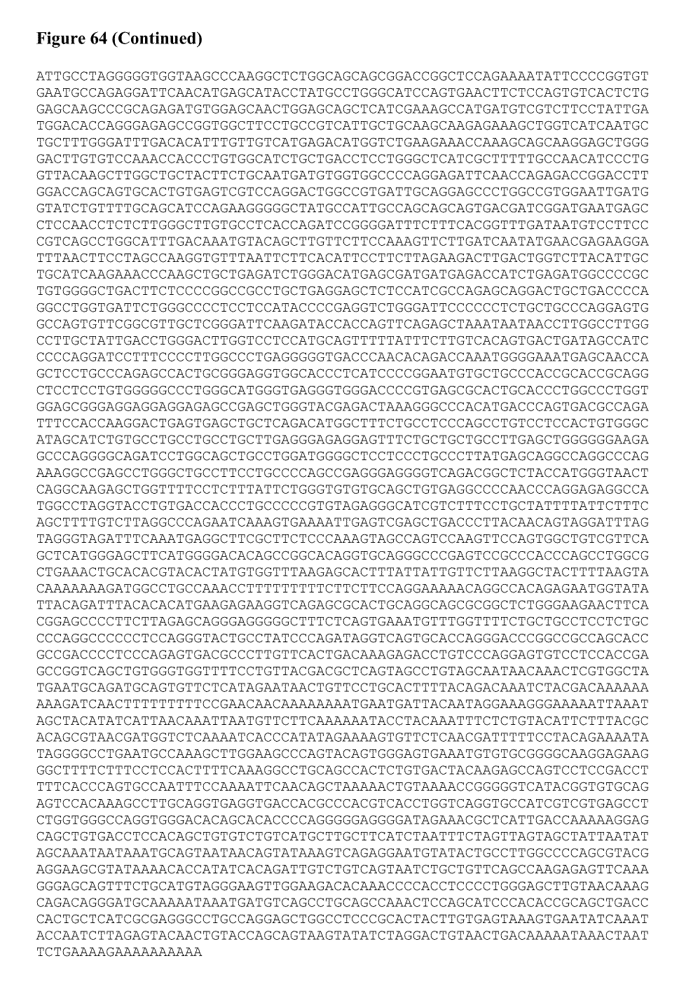

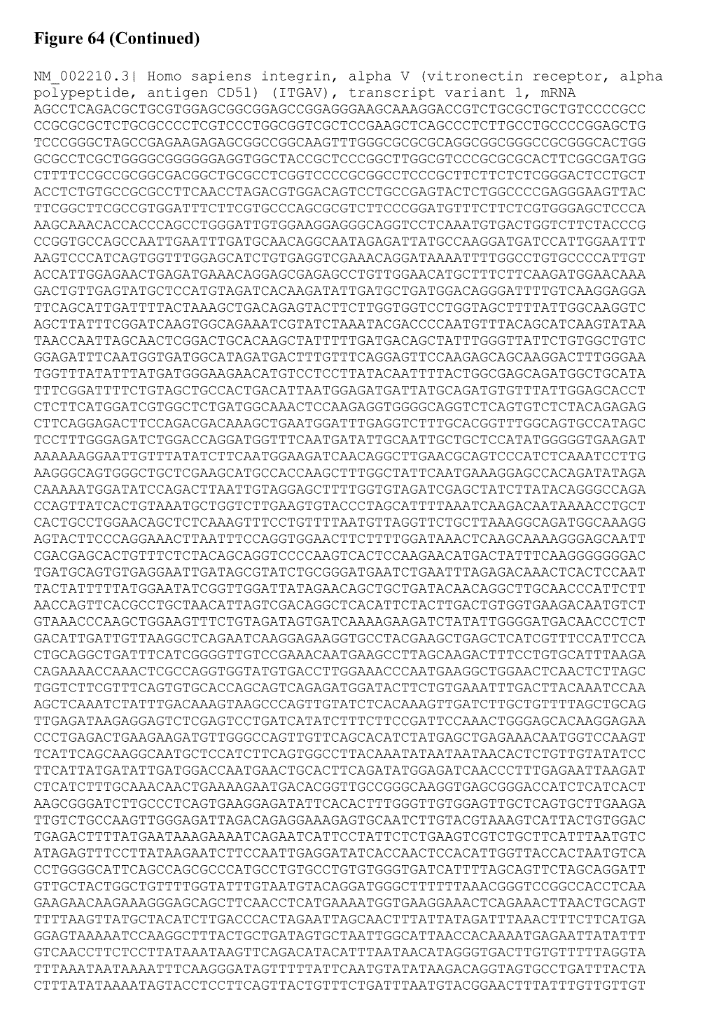

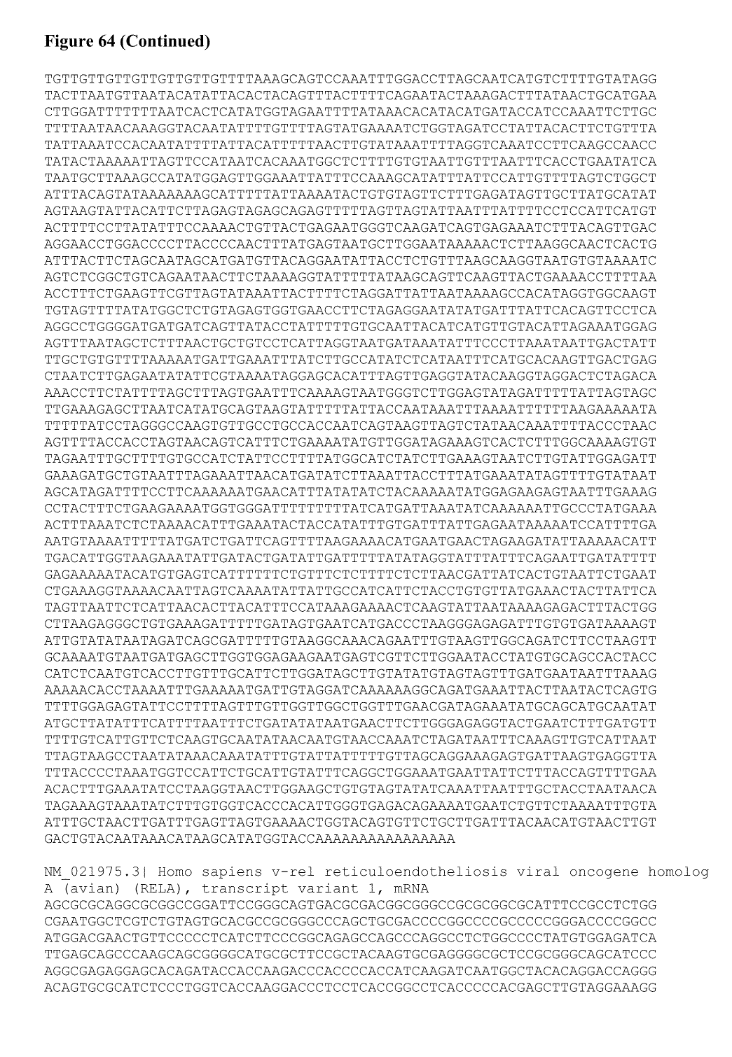

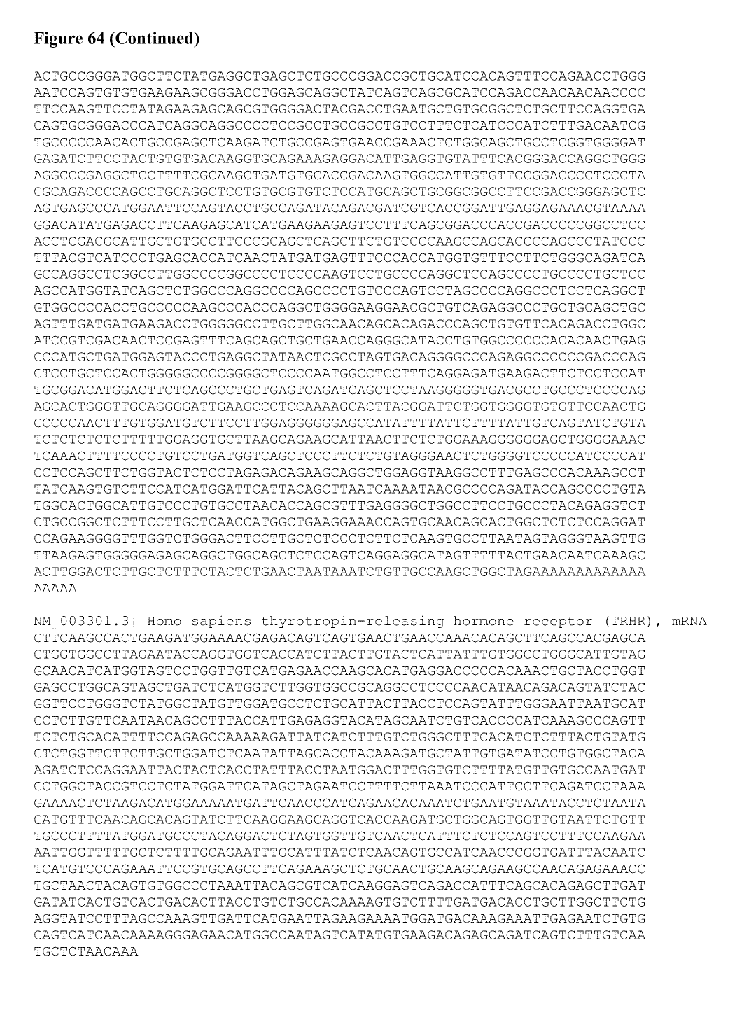

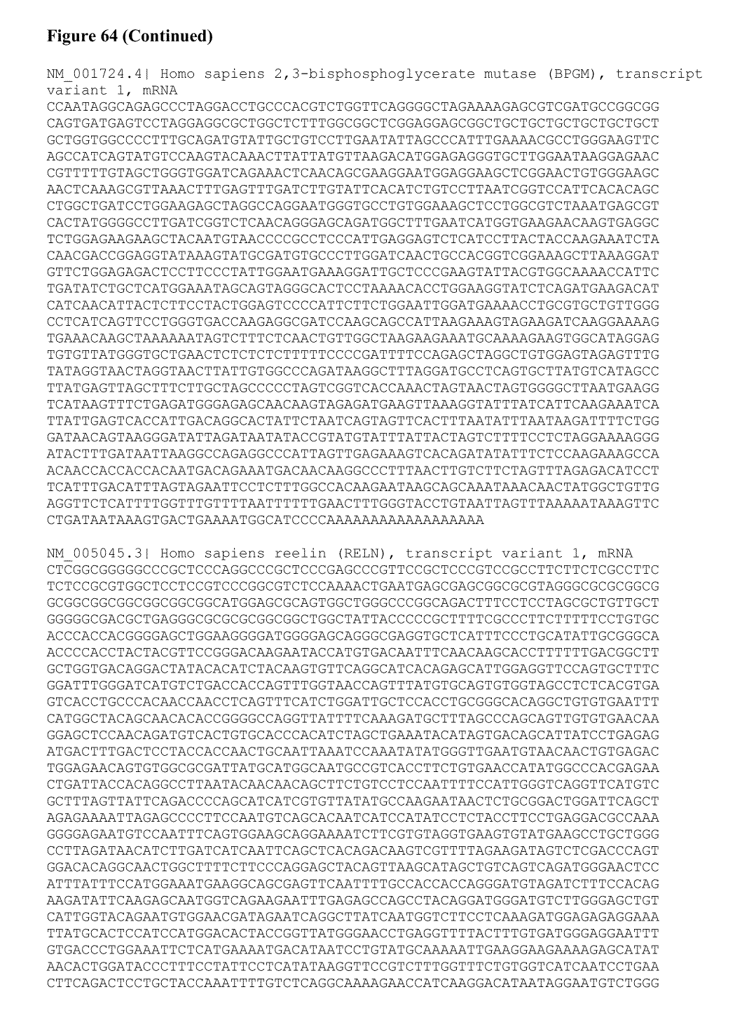

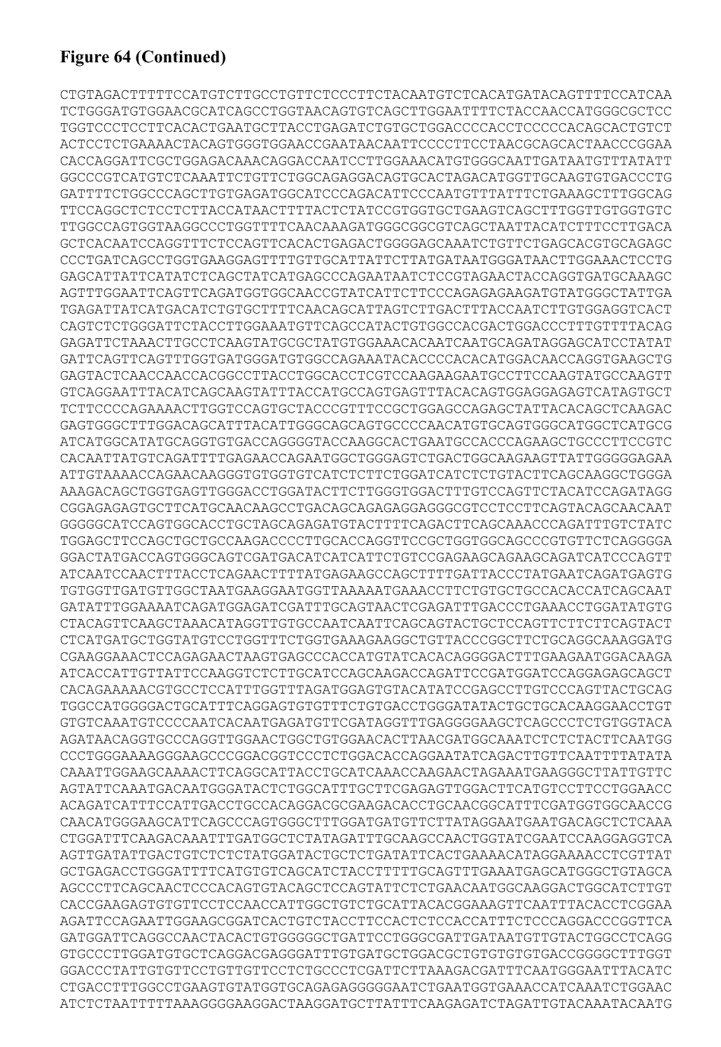

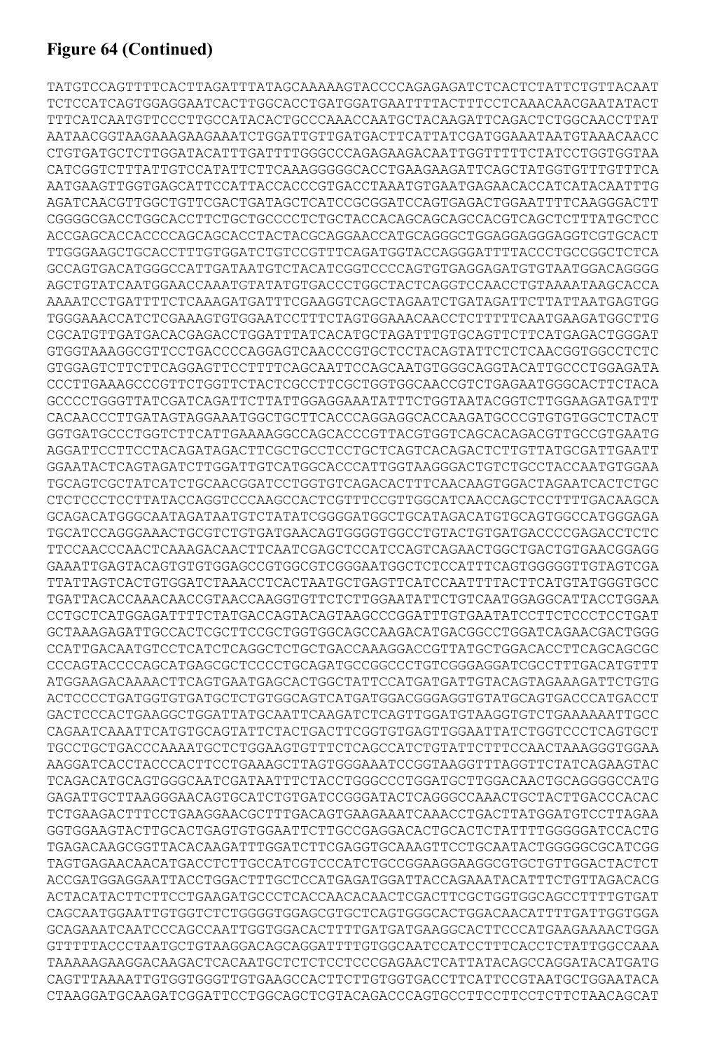

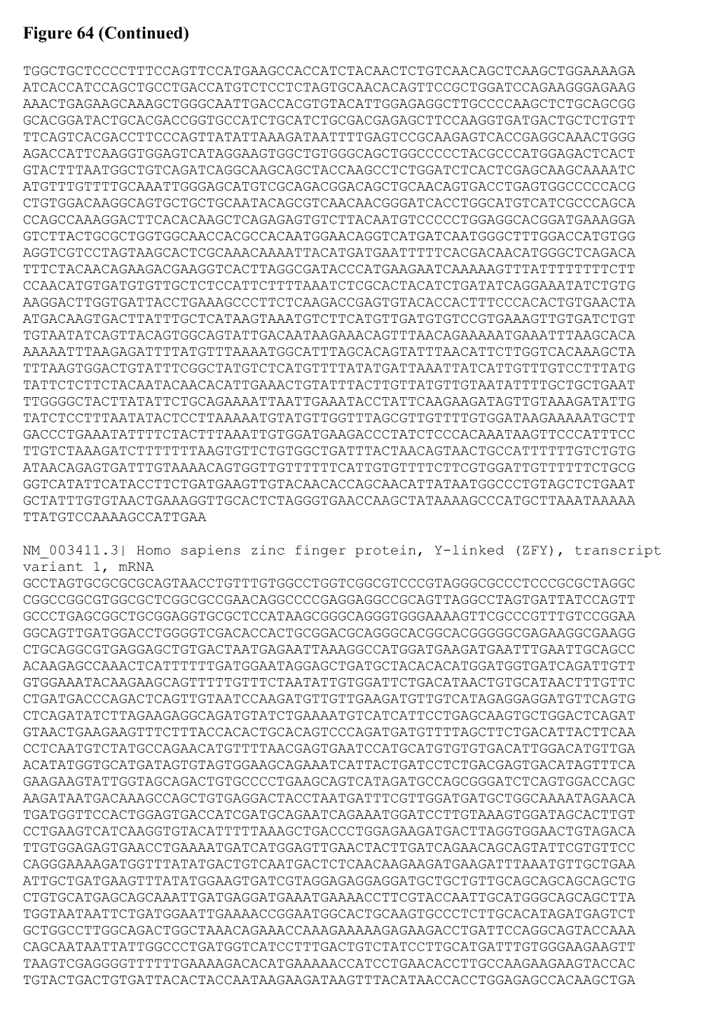

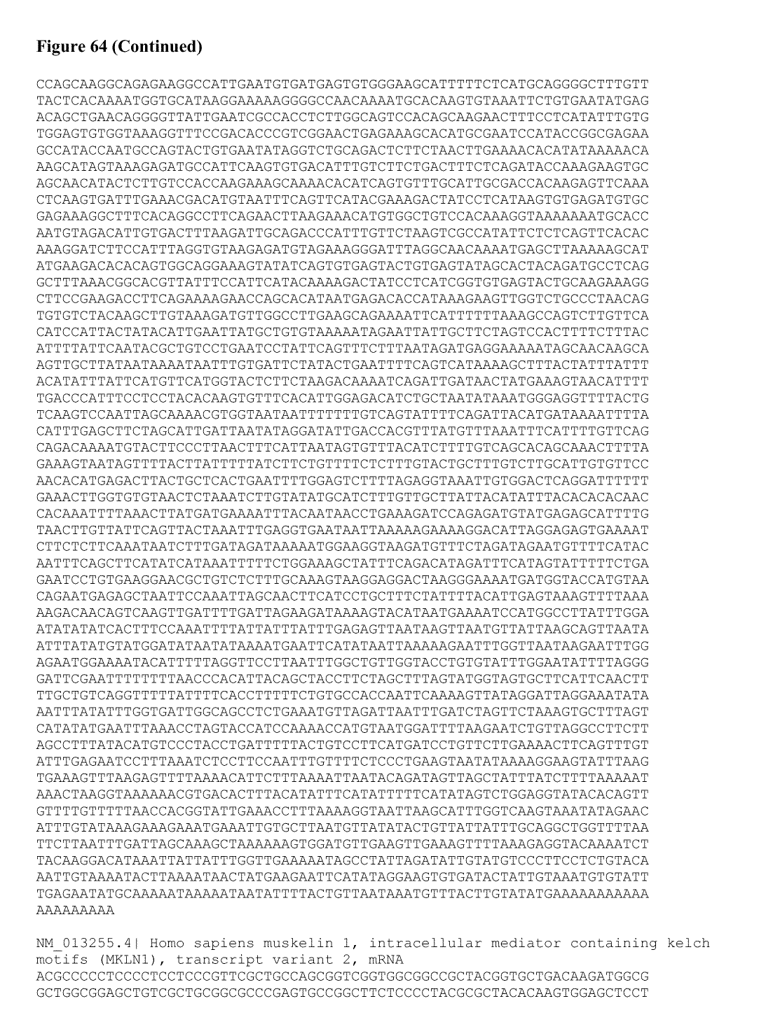

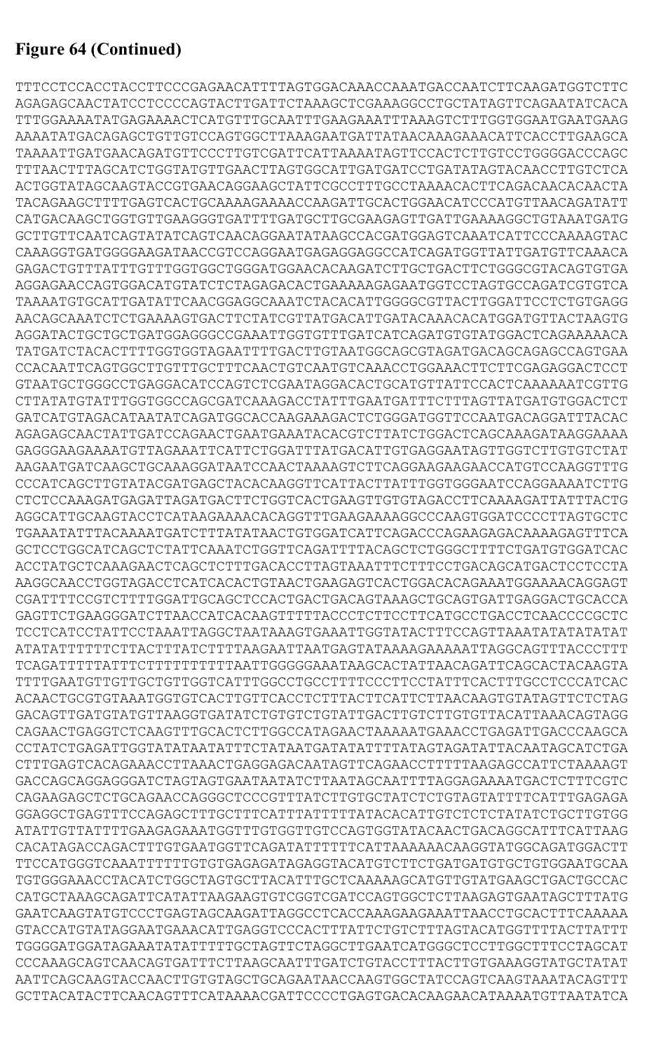

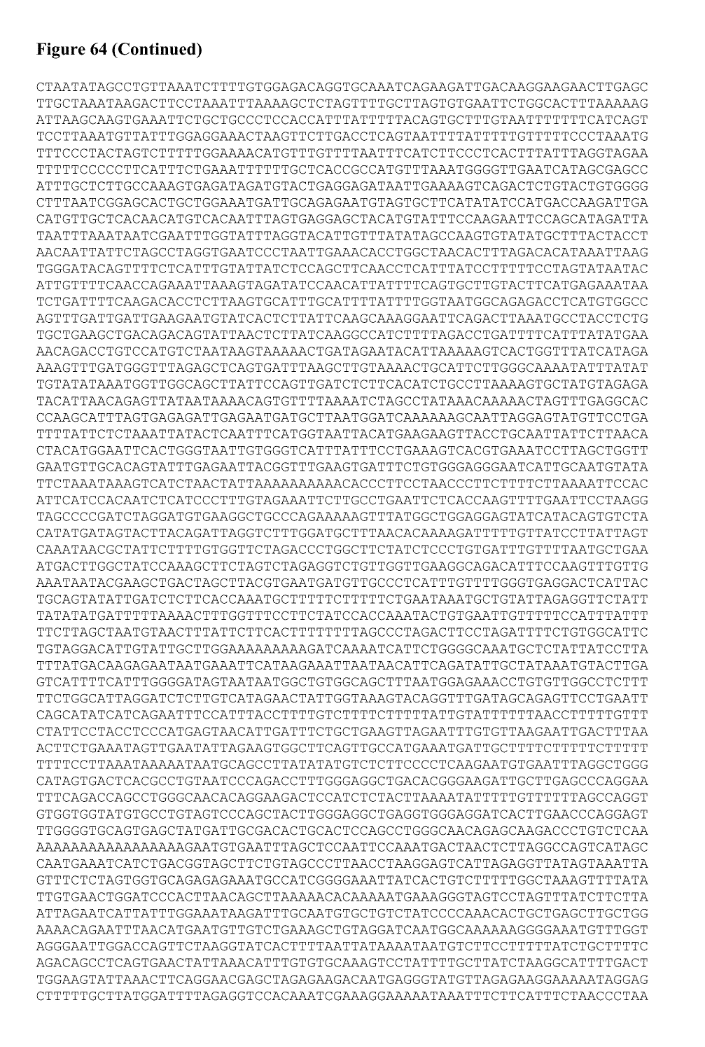

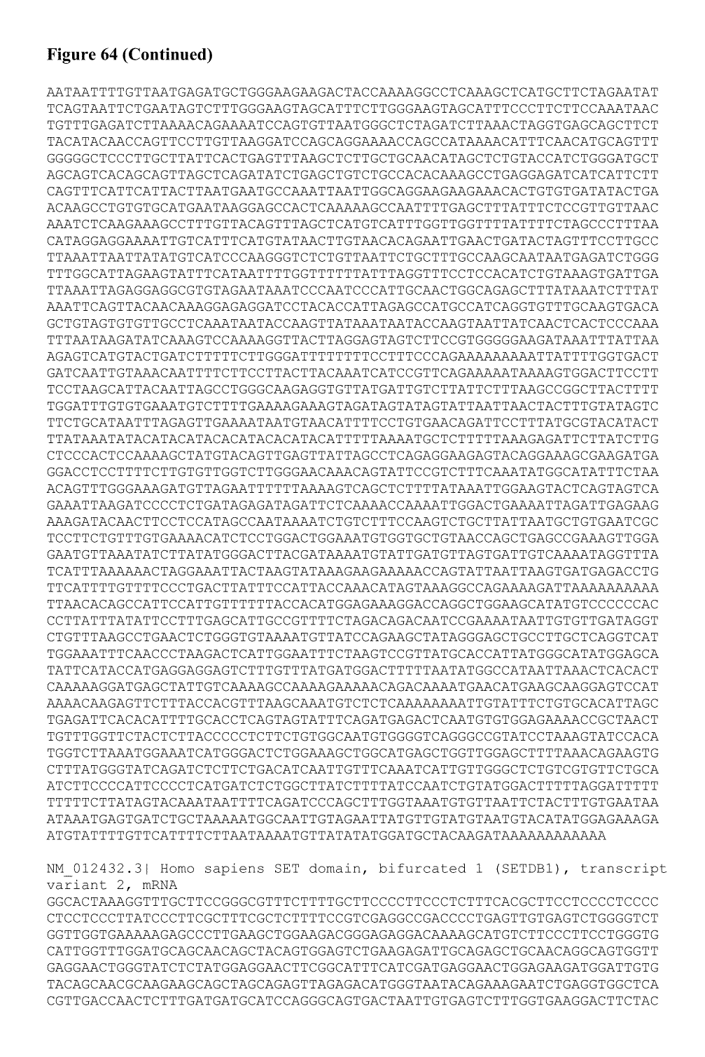

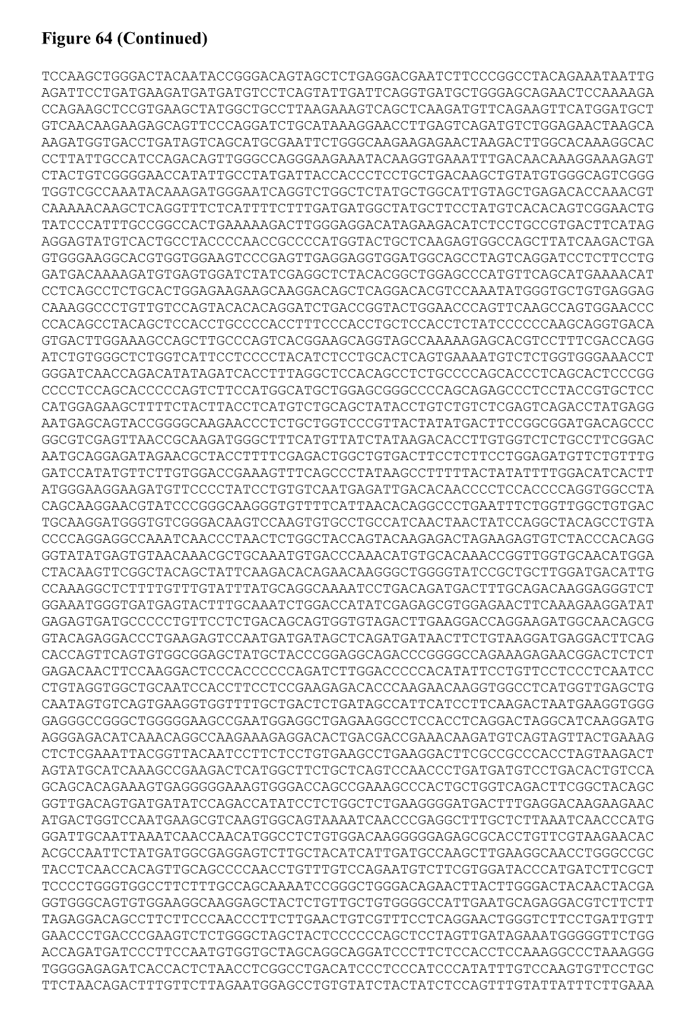

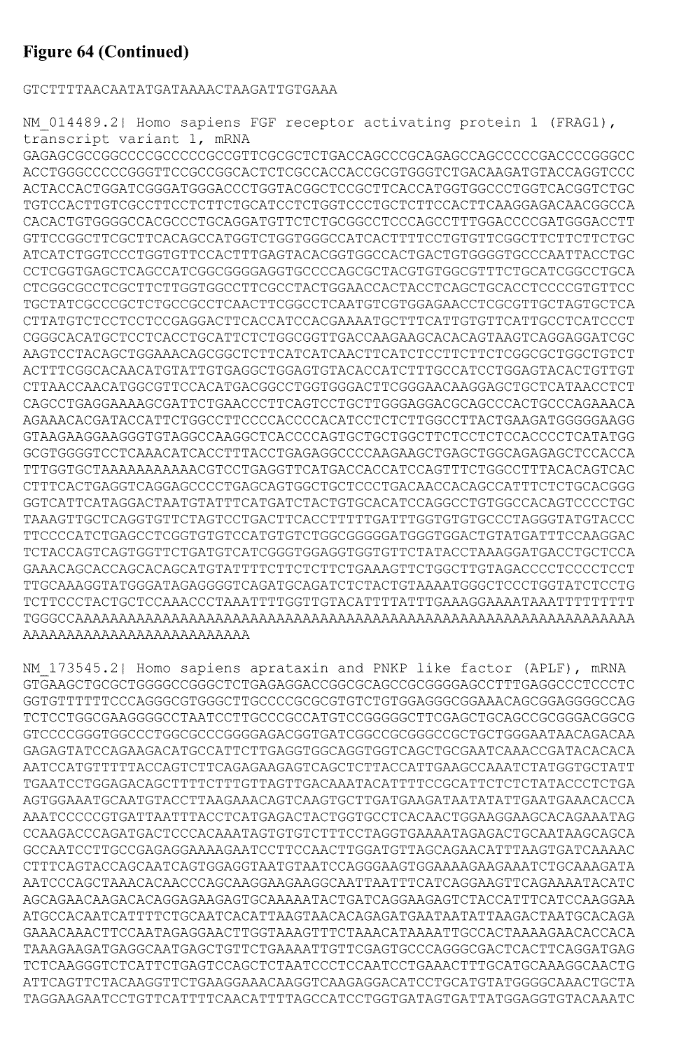

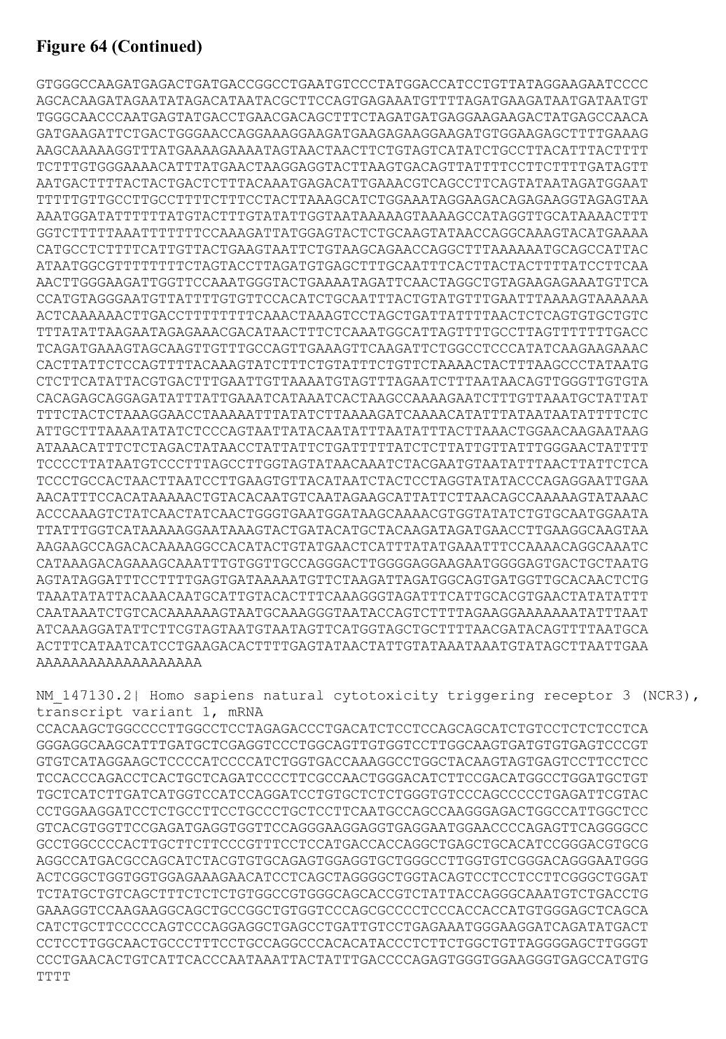

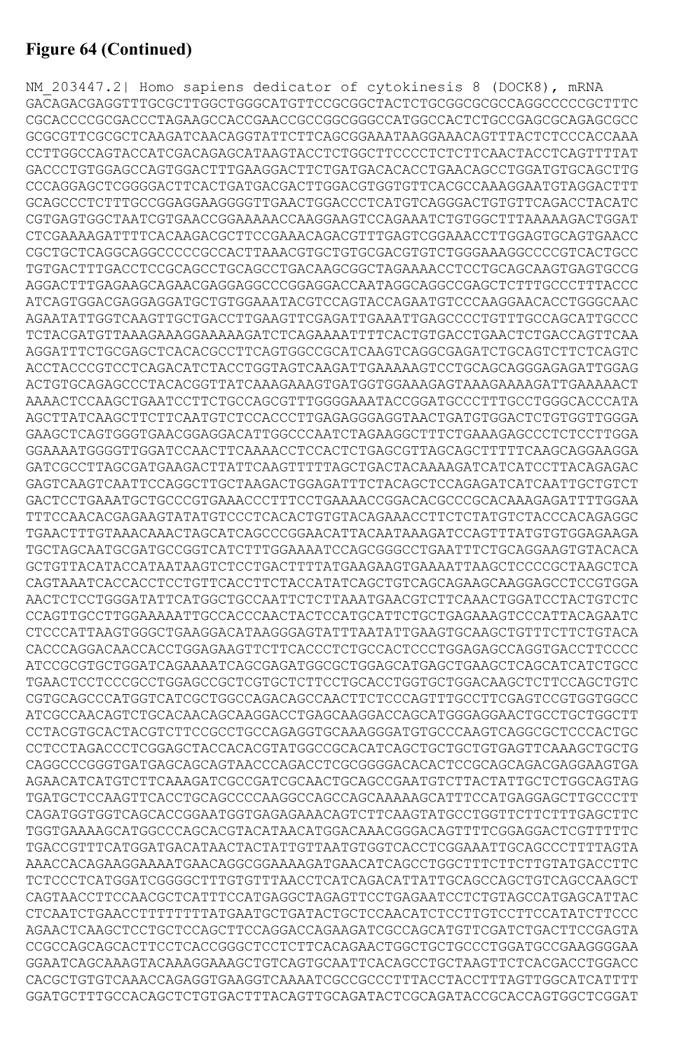

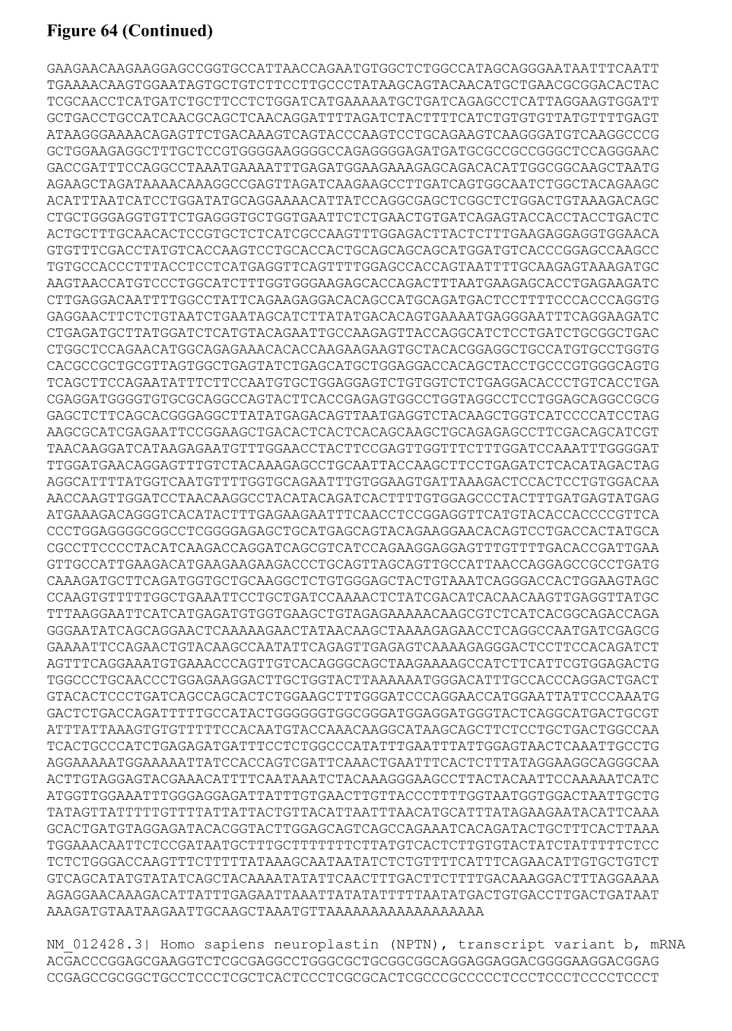

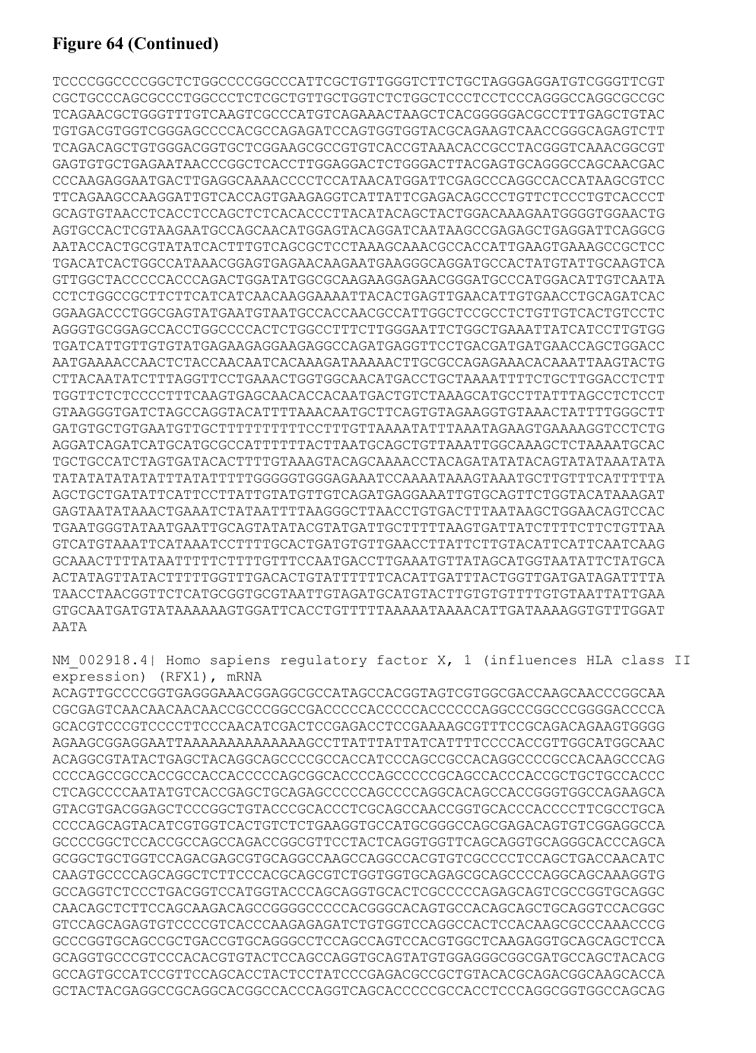

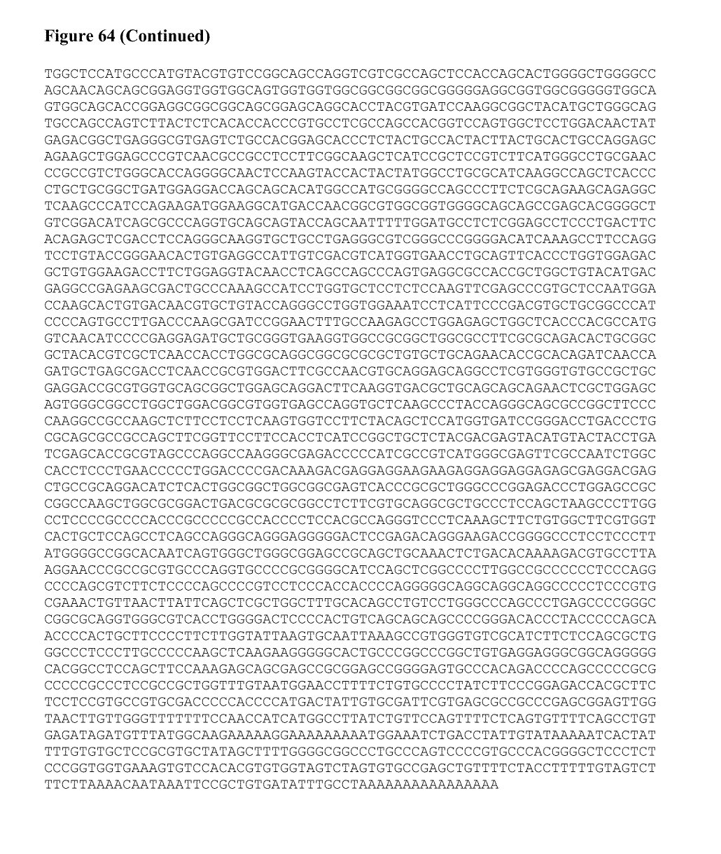

Agents that inhibit the activity of autophagy-inhibiting gene products are useful, for example, in enhancing autophagy and in the treatment of neurodegenerative diseases. Examples of such inhibitors of autophagy-inhibiting gene products are listed in Table 7 and FIG. 63.

TABLE 7
Agents that inhibit autophagy-inhibiting gene products.

| Target Gene | | |
|---|---|---|
| Symbol | Target Gene Name | Agent |
| TH | tyrosine hydroxylase; TH | alpha-methyl- |
| | | para-tyrosine |
| | | (Metyrosine) |
| FGFR1 | fibroblast growth factor receptor 1 (fms- | TK1258 |
| | related tyrosine kinase 2, Pfeiffer | (CHIR258) |
| | syndrome); FGFR1 | |
| AGER | advanced glycosylation end product- | PF 04494700 |
| | specific receptor; AGER | (TTP488) |
| C5AR1 | complement component 5a receptor | PMX53 |
| | 1; C5AR1 | |
| ADRA1A | adrenergic, alpha-1A-, receptor; ADRA1A | Tamsulosin |
| ADRA1A | adrenergic, alpha-1A-, receptor; ADRA1A | Doxazosin |
| ADRA1A | adrenergic, alpha-1A-, receptor; ADRA1A | Prazosin |
| | | hydrochloride |
| ADRA1A | adrenergic, alpha-1A-, receptor; ADRA1A | alfuzosin |
| | | hydrochloride |
| UTS2R | urotensin 2 receptor; UTS2R | Urotensin II |
| CHRND | cholinergic receptor, nicotinic, | Galantamine |
| | delta; CHRND | (Galanthamine) |
| CHRND | cholinergic receptor, nicotinic, | Mecamylamine |
| | delta; CHRND | hydrochloride |
| | | (Inversine) |
| CASP1 | caspase 1, apoptosis-related cysteine | Pralnacasan |
| | peptidase (interleukin 1, beta, | (VX-740, |
| | convertase); CASP1 | HMR |
| | | 3480) |
| PRKCA | protein kinase C, alpha; PRKCA | ISIS 3521 |
| | | (carboplatin, |
| | | paclitaxel) |
| PRKCA | protein kinase C, alpha; PRKCA | Gemcitabine; |
| PRKCA | protein kinase C, alpha; PRKCA | LY900003 |
| AURKA | aurora kinase A; AURKA | MK-5108 |
| PLCH2 | phospholipase C, eta 2; PLCH2 | U73122 |
| PLCH2 | phospholipase C, eta 2; PLCH2 | D609 |

Alternatively, agents that enhance the activity of autophagy-inhibiting gene products are useful, for example, in inhibiting autophagy and in the treatment of cancer and pancreatitis. Examples of such enhancers of autophagy-inhibiting gene products are listed in Table 8 and FIG. 63.

TABLE 8
Agents that enhance autophagy-inhibiting gene products.

| Target | | |
|---|---|---|
| Gene | | |
| Symbol | Target Gene Name | Agent |
| FGFR1 | fibroblast growth factor receptor 1 (fms- | Cardio Vascu- |
| | related tyrosine kinase 2, Pfeiffer | Grow (FGF-1, |
| | syndrome); FGFR1 | CVBT-141) |
| FGFR1 | fibroblast growth factor receptor 1 (fms- | Acidic FGF |
| | related tyrosine kinase 2, Pfeiffer | (aFGF); |
| | syndrome); FGFR1 | |
| FGFR1 | fibroblast growth factor receptor 1 (fms- | XRP0038 |
| | related tyrosine kinase 2, Pfeiffer | (NV1FGF) |
| | syndrome); FGFR1 | |
| FGFR1 | fibroblast growth factor receptor 1 (fms- | Rh-aFGF |
| | related tyrosine kinase 2, Pfeiffer | |
| | syndrome); FGFR1 | |
| PPARD | peroxisome proliferator-activated receptor | GW501516 |
| | delta; PPARD | |
| GHSR | growth hormone secretagogue | Ibutamoren |
| | receptor; GHSR | Mesylate |
| | | (MK-0677) |
| GHSR | growth hormone secretagogue | KP-102LN |
| | receptor; GHSR | |
| GHSR | growth hormone secretagogue | EP1572 (ghrelin |
| | receptor; GHSR | agonist) |
| TRHR | thyrotropin-releasing hormone | TRH |
| | receptor; TRHR | |
| TRHR | thyrotropin-releasing hormone | S-0373 |
| | receptor; TRHR | (KPS-0373) |
| TRHR | thyrotropin-releasing hormone | S-14820 |
| | receptor; TRHR | |
| TLR3 | toll-like receptor 3; TLR3 | Poly-ICR |
| TLR3 | toll-like receptor 3; TLR3 | CQ-07001 |
| PRKAA2 | protein kinase, AMP-activated, alpha 2 | cryptotanshinone |
| | catalytic subunit; PRKAA2 | |

Further examples of agents that modulate the autophagy-related gene products listed in tables 1-4 can be found in, for example, U.S. Pat. Nos. 7,348,140; 6,982,265; 6,723,694; 6,617,311; 6,372,250; 6,334,998; 6,319,905; 6,312,949; 6,297,238; 6,228,835; 6,214,334; 6,096,778; 5,990,083; 5,834,457; 5,783,683; 5,681,747; 5,556,837; 5,464,614, each of which is hereby specifically incorporated by reference in its entirety. Examples of agents that modulate the autophagy-related gene products listed in tables 1-4 can also be found in, for example, U.S. Patent Application Publication Numbers: US2009/0137572; US2009/0136475; US2009/0105149; US2009/0088401; US2009/0087454; US2009/0087410; US2009/0075900; US2009/0074774; US2009/0074711; US2009/0074676; US2009/0069245; US2009/0068194; US2009/0068168; US2009/0060898; US2009/0047240; US2009/0042803; US2009/0029992; US2009/0011994; US2009/0005431; US2009/0005309; US2009/0004194; US2008/0319026; US2008/0312247; US2008/0300316; US2008/0300180; US2008/0299138; US2008/0280991; US2008/0280886; US2008/0268071; US2008/0262086; US2008/0255200; US2008/0255084; US2008/0255036; US2008/0242687; US2008/0241289; US2008/0234284; US2008/0234257; US2008/0221132; US2008/0194672; US2008/0194555; US2008/0187490; US2008/0171769; US2008/0167312; US2008/0146573; US2008/0132555; US2008/0125386; US2008/0124379; US2008/0103189; US2008/0051465; US2008/0051383; US2008/0045588; US2008/0045561; US2008/0045558; US2008/0039473; US2008/0033056; US2008/0021036; US2008/0021029; US2008/0004300; US2007/0293525; US2007/0293494; US2007/0287734; US2007/0286853; US2007/0281965; US2007/0281894; US2007/0280886; US2007/0274981; US2007/0259891; US2007/0259827; US2007/0254877; US2007/0249519; US2007/0248605; US2007/0219235; US2007/0219114; US2007/0203064; US2007/0173440; US2007/0155820; US2007/0149622; US2007/0149580; US2007/0134273; US2007/0129389; US2007/0112031; US2007/0099964; US2007/0099952; US2007/0098716; US2007/0093480; US2007/0082929; US2007/0004765; US2007/0004654; US2006/0286102; US2006/0276381; US2006/0265767; US2006/0263368; US2006/0257867; US2006/0223742; US2006/0211752; US2006/0199796; US2006/0194821; US2006/0166871; US2006/0147456; US2006/0134128; US2006/0115475; US2006/0110746; US2006/0058255; US2006/0025566; US2006/0009454; US2006/0009452; US2006/0002866; US2005/0288316; US2005/0288243; US2005/0250719; US2005/0249751; US2005/0246794; US2005/0227921; US2005/0222171; US2005/0197341; US2005/0187237; US2005/0182006; US2005/0175581; US2005/0171182; US2005/0164298; US2005/0153955; US2005/0153878; US2005/0148511; US2005/0143381; US2005/0119273; US2005/0106142; US2005/0096363; US2005/0070493; US2005/0043233; US2005/0043221; US2005/0038049; US2005/0015263; US2005/0009870; US2004/0266777; US2004/0261190; US2004/0248965; US2004/0248884; US2004/0242559; US2004/0241797; US2004/0229250; US2004/0220270; US2004/0204368; US2004/0192629; US2004/0186157; US2004/0132648; US2004/0091919; US2004/0072836; US2004/0063708; US2004/0063707; US2004/0057950; US2003/0225098; US2003/0220246; US2003/0211967; US2003/0199525; US2003/0187001; US2003/0186844; US2003/0166574; US2003/0166573; US2003/0166001; US2003/0153752; US2003/0077298; US2003/0069430; US2003/0059455; US2003/0040612; US2009/0099069; US2008/0312413; US2008/0280845; US2008/0248462; US2008/0248462; US2008/0213250; US2008/0145313; US2008/0021080; US2008/0021036; US2008/0004309; US2007/0298124; US2007/0298104; US2007/0281986; US2007/0264195; US2007/0232556; US2007/0190149; US2007/0111934; US2007/0071675; US2007/0021360; US2007/0010658; US2006/0235034; US2006/0233799; US2006/0160737; US2006/0128696; US2006/0121042; US2006/0039904; US2006/0019882; US2005/0272655; US2005/0197293; US2004/0247592; US2004/0204356; US2004/0132023; US2004/0116669; US2004/0072836; US2004/0048895; US2004/0022765; US2003/0165485; US2003/0162964; US2003/0153503; US2003/0125276; US2003/0114657; US2003/0091569; US2003/0078199; US2002/0137095; US2001/0006793; US2001/0002393; US2002/0183319; and US2002/0156081, each of which is hereby specifically incorporated by reference in its entirety.

### 4. OLIGONUCLEOTIDE INHIBITORS OF AUTOPHAGY-RELATED GENE PRODUCTS

In certain embodiments of the present invention, oligonucleotide inhibitors of autophagy-related RNA gene products are used to modulate autophagy and to treat autophagy-related diseases. Oligonucleotide inhibitors include, but are not limited to, antisense molecules, siRNA molecules, shRNA molecules, ribozymes and triplex molecules. Such molecules are known in the art and the skilled artisan would be able to create oligonucleotide inhibitors for any of the autophagy-related genes of the invention using routine methods.

Antisense molecules, siRNA or shRNA molecules, ribozymes or triplex molecules may be contacted with a cell or administered to an organism. Alternatively, constructs encoding such molecules may be contacted with or introduced into a cell or organism. Antisense constructs, antisense oligonucleotides, RNA interference constructs or siRNA duplex RNA molecules can be used to interfere with expression of a protein of interest, e.g., an autophagy-related gene of the present invention. Typically at least 15, 17, 19, or 21 nucleotides of the complement of the mRNA sequence are sufficient for an antisense molecule. Typically at least 15, 19, 21, 22, or 23 nucleotides of a target sequence are sufficient for an RNA interference molecule. In some embodiments, an RNA interference molecule will have a 2 nucleotide 3′ overhang. If the RNA interference molecule is expressed in a cell from a construct, for example from a hairpin molecule or from an inverted repeat of the desired autophagy-related gene sequence, then the endogenous cellular machinery may create the overhangs. siRNA molecules can be prepared by chemical synthesis, in vitro transcription, or digestion of long dsRNA by Rnase III or Dicer. These can be introduced into cells by transfection, electroporation, intracellular infection or other methods known in the art. See, for example: Hannon, G J, 2002, RNA Interference, Nature 418: 244-251; Bernstein E et al., 2002, The rest is silence. RNA 7: 1509-1521; Hutvagner G et al., RNAi: Nature abhors a double-strand. Cur. Open. Genetics & Development 12: 225-232; Brummelkamp, 2002, A system for stable expression of short interfering RNAs in mammalian cells. Science 296: 550-553; Lee N S, Dohjima T, Bauer G, Li H, Li M-J, Ehsani A, Salvaterra P, and Rossi J. (2002). Expression of small interfering RNAs targeted against HIV-1 rev transcripts in human cells. Nature Biotechnol. 20:500-505; Miyagishi M, and Taira K. (2002). U6-promoter-driven siRNAs with four uridine 3′ overhangs efficiently suppress targeted gene expression in mammalian cells. Nature Biotechnol. 20:497-500; Paddison P J, Caudy A A, Bernstein E, Hannon G J, and Conklin D S. (2002). Short hairpin RNAs (shRNAs) induce sequence-specific silencing in mammalian cells. Genes & Dev. 16:948-958; Paul C P, Good P D, Winer I, and Engelke D R. (2002). Effective expression of small interfering RNA in human cells. Nature Biotechnol. 20:505-508; Sui G, Soohoo C, Affar E-B, Gay F, Shi Y, Forrester W C, and Shi Y. (2002). A DNA vector-based RNAi technology to suppress gene expression in mammalian cells. Proc. Natl. Acad. Sci. USA 99(6):5515-5520; Yu J-Y, DeRuiter S L, and Turner D L. (2002). RNA interference by expression of short-interfering RNAs and hairpin RNAs in mammalian cells. Proc. Natl. Acad. Sci. USA 99(9):6047-6052, PCT publications WO2006/066048 and WO2009/029688, US published application US2009/0123426, each of which is incorporated by reference in its entirety.

Antisense or RNA interference molecules can be delivered in vitro to cells or in vivo, e.g., to tumors or diseased tissues of a mammal Typical delivery means known in the art can be used. For example, delivery to a tumor can be accomplished by intratumoral injections. Other modes of delivery can be used without limitation, including: intravenous, intramuscular, intraperitoneal, intraarterial, local delivery during surgery, endoscopic, subcutaneous, and per os. Vectors can be selected for desirable properties for any particular application. Vectors can be viral, bacterial or plasmid. Adenoviral vectors are useful in this regard. Tissue-specific, cell-type specific, or otherwise regulatable promoters can be used to control the transcription of the inhibitory polynucleotide molecules. Non-viral carriers such as liposomes or nanospheres can also be used.

In the present methods, a RNA interference molecule or an RNA interference encoding oligonucleotide can be administered to the subject, for example, as naked RNA, in combination with a delivery reagent, and/or as a nucleic acid comprising sequences that express the siRNA or shRNA molecules. In some embodiments the nucleic acid comprising sequences that express the siRNA or shRNA molecules are delivered within vectors, e.g. plasmid, viral and bacterial vectors. Any nucleic acid delivery method known in the art can be used in the present invention. Suitable delivery reagents include, but are not limited to, e.g, the Mims TRANSIT-TKO® lipophilic reagent; LIPOFECTIN® transfection reagent; LIPOFECTAMINE® transfection reagent; cellfectin; polycations (e.g., polylysine), atelocollagen, nanoplexes and liposomes.

The use of atelocollagen as a delivery vehicle for nucleic acid molecules is described in Minakuchi et al. Nucleic Acids Res., 32(13):e109 (2004); Hanai et al. Ann NY Acad. Sci., 1082:9-17 (2006); and Kawata et al. Mol Cancer Ther., 7(9):2904-12 (2008); each of which is incorporated herein in their entirety.

In some embodiments of the invention, liposomes are used to deliver an inhibitory oligonucleotide to a subject. Liposomes suitable for use in the invention can be formed from standard vesicle-forming lipids, which generally include neutral or negatively charged phospholipids and a sterol, such as cholesterol. The selection of lipids is generally guided by consideration of factors such as the desired liposome size and half-life of the liposomes in the blood stream. A variety of methods are known for preparing liposomes, for example, as described in Szoka et al. (1980), Ann. Rev. Biophys. Bioeng. 9:467; and U.S. Pat. Nos. 4,235,871, 4,501,728, 4,837,028, and 5,019,369, the entire disclosures of which are herein incorporated by reference.

The liposomes for use in the present methods can comprise a ligand molecule that targets the liposome to cancer cells, pancreatic cells or neurons. Ligands which bind to receptors prevalent in cancer cells, pancreatic cells or neurons, such as monoclonal antibodies that bind to cell-type specific antigens, are preferred.

The liposomes for use in the present methods can also be modified so as to avoid clearance by the mononuclear macrophage system (“MMS”) and reticuloendothelial system (“RES”). Such modified liposomes have opsonization-inhibition moieties on the surface or incorporated into the liposome structure. In an embodiment, a liposome of the invention can comprise both opsonization-inhibition moieties and a ligand.

Opsonization-inhibiting moieties for use in preparing the liposomes of the invention are typically large hydrophilic polymers that are bound to the liposome membrane. As used herein, an opsonization inhibiting moiety is “bound” to a liposome membrane when it is chemically or physically attached to the membrane, e.g., by the intercalation of a lipid-soluble anchor into the membrane itself, or by binding directly to active groups of membrane lipids. These opsonization-inhibiting hydrophilic polymers form a protective surface layer that significantly decreases the uptake of the liposomes by the MMS and RES; e.g., as described in U.S. Pat. No. 4,920,016, the entire disclosure of which is herein incorporated by reference.

Opsonization inhibiting moieties suitable for modifying liposomes are preferably water-soluble polymers with a number-average molecular weight from about 500 to about 40,000 daltons, and more preferably from about 2,000 to about 20,000 daltons. Such polymers include polyethylene glycol (PEG) or polypropylene glycol (PPG) derivatives; e.g., methoxy PEG or PPG, and PEG or PPG stearate; synthetic polymers such as polyacrylamide or poly N-vinyl pyrrolidone; linear, branched, or dendrimeric polyamidoamines; polyacrylic acids; polyalcohols, e.g., polyvinylalcohol and polyxylitol to which carboxylic or amino groups are chemically linked, as well as gangliosides, such as ganglioside GM1. Copolymers of PEG, methoxy PEG, or methoxy PPG, or derivatives thereof, are also suitable. In addition, the opsonization inhibiting polymer can be a block copolymer of PEG and either a polyamino acid, polysaccharide, polyamidoamine, polyethyleneamine, or polynucleotide. The opsonization inhibiting polymers can also be natural polysaccharides containing amino acids or carboxylic acids, e.g., galacturonic acid, glucuronic acid, mannuronic acid, hyaluronic acid, pectic acid, neuraminic acid, alginic acid, carrageenan; aminated polysaccharides or oligosaccharides (linear or branched); or carboxylated polysaccharides or oligosaccharides, e.g., reacted with derivatives of carbonic acids with resultant linking of carboxylic groups. Preferably, the opsonization-inhibiting moiety is a PEG, PPG, or derivatives thereof. Liposomes modified with PEG or PEG-derivatives are sometimes called “PEGylated liposomes.”

The opsonization inhibiting moiety can be bound to the liposome membrane by any one of numerous well-known techniques. For example, an N-hydroxysuccinimide ester of PEG can be bound to a phosphatidyl-ethanolamine lipid-soluble anchor, and then bound to a membrane. Similarly, a dextran polymer can be derivatized with a stearylamine lipid-soluble anchor via reductive amination using Na(CN)BH₃ and a solvent mixture, such as tetrahydrofuran and water in a 30:12 ratio at 60° C.

Liposomes modified with opsonization-inhibition moieties remain in the circulation much longer than unmodified liposomes. For this reason, such liposomes are sometimes called “stealth” liposomes. Stealth liposomes are known to accumulate in tissues fed by porous or “leaky” microvasculature. Thus, tissue characterized by such microvasculature defects, for example solid tumors, will efficiently accumulate these liposomes; see Gabizon, et al. (1988), Proc. Natl. Acad. Sci., USA, 18:6949-53. In addition, the reduced uptake by the RES lowers the toxicity of stealth liposomes by preventing significant accumulation of the liposomes in the liver and spleen.

### 5. ANTIBODIES SPECIFIC FOR AUTOPHAGY-RELATED GENE PRODUCTS

Because of their ability to bind to a particular target with high specificity, antibodies specific for polypeptide autophagy-related gene products are able to either inhibit or enhance the activities of such gene products and thereby inhibit or enhance autophagy. For example, in some embodiments, an antibody specific for a receptor can inhibit the activity of the receptor by blocking its interaction with an activating ligand. Likewise, antibodies specific for a soluble ligand (e.g. a cytokine or growth factor) or a membrane-bound ligand can inhibit the activity of a receptor that is capable of binding to the ligand by inhibiting the binding of the ligand to the receptor. In other embodiments, antibodies specific for a receptor can be used to cross-link and thereby activate the receptor. Though antibodies are particularly useful in inhibiting or enhancing the activity extracellular proteins (e.g., receptors and/or ligands), the use of intracellular antibodies to inhibit protein function in a cell is also known in the art (see e.g., Carlson, J. R. (1988) Mol. Cell. Biol. 8:2638-2646; Biocca, S. et al. (1990) EMBO J. 9:101-108; Werge, T. M. et al. (1990) FEBS Lett. 274:193-198; Carlson, J. R. (1993) Proc. Natl. Acad. Sci. USA 90:7427-7428; Marasco, W. A. et al. (1993) Proc. Natl. Acad. Sci. USA 90:7889-7893; Biocca, S. et al. (1994) Biotechnology (NY) 12:396-399; Chen, S-Y. et al. (1994) Hum. Gene Ther. 5:595-601; Duan, L et al. (1994) Proc. Natl. Acad. Sci. USA 91:5075-5079; Chen, S-Y. et al. (1994) Proc. Natl. Acad. Sci. USA 91:5932-5936; Beerli, R. R. et al. (1994) J. Biol. Chem. 269:23931-23936; Beerli, R. R. et al. (1994) Biochem. Biophys. Res. Commun. 204:666-672; Mhashilkar, A. M. et al. (1995) EMBO J. 14:1542-1551; Richardson, J. H. et al. (1995) Proc. Natl. Acad. Sci. USA 92:3137-3141; PCT Publication No. WO 94/02610 by Marasco et al.; and PCT Publication No. WO 95/03832 by Duan et al.). Therefore, antibodies specific for peptide products of autophagy-related genes are useful as biological agents for the methods of the present invention.

Antibodies that specifically bind to a peptide product of an autophagy-related gene can be produced using a variety of known techniques, such as the standard somatic cell hybridization technique described by Kohler and Milstein, Nature 256: 495 (1975). Additionally, other techniques for producing monoclonal antibodies known in the art can also be employed, e.g., viral or oncogenic transformation of B lymphocytes, phage display technique using libraries of human antibody genes.

Polyclonal antibodies can be prepared by immunizing a suitable subject with a polypeptide immunogen. The polypeptide antibody titer in the immunized subject can be monitored over time by standard techniques, such as with an enzyme linked immunosorbent assay (ELISA) using immobilized polypeptide. If desired, the antibody directed against the antigen can be isolated from the mammal (e.g., from the blood) and further purified by well known techniques, such as protein A chromatography to obtain the IgG fraction. At an appropriate time after immunization, e.g., when the antibody titers are highest, antibody-producing cells can be obtained from the subject and used to prepare monoclonal antibodies.

Any of the many well known protocols used for fusing lymphocytes and immortalized cell lines can be applied for the purpose of generating monoclonal antibodies specific against the products of autophagy-related genes (see, e.g., Galfre, G. et al. (1977) Nature 266:55052; Gefter et al. (1977) supra; Lerner (1981) supra; Kenneth (1980) supra). Moreover, the ordinary skilled worker will appreciate that there are many variations of such methods which also would be useful. Typically, an immortal cell line (e.g., a myeloma cell line) is derived from the same mammalian species as the lymphocytes. For example, murine hybridomas can be made by fusing lymphocytes from a mouse immunized with an immunogenic preparation of the present invention with an immortalized mouse cell line. An example of an appropriate mouse cell lines are mouse myeloma cell lines that are sensitive to culture medium containing hypoxanthine, aminopterin and thymidine (“HAT medium”). Any of a number of myeloma cell lines can be used as a fusion partner according to standard techniques, e.g., the P3-NS1/1-Ag4-1, P3-x63-Ag8.653 or Sp2/O—Ag14 myeloma lines. These myeloma lines are available from the American Type Culture Collection (ATCC), Rockville, Md. Typically, HAT-sensitive mouse myeloma cells are fused to mouse splenocytes using polyethylene glycol (“PEG”). Hybridoma cells resulting from the fusion are then selected using HAT medium, which kills unfused and unproductively fused myeloma cells (unfused splenocytes die after several days because they are not transformed). Hybridoma cells producing a monoclonal antibody of the invention are detected by screening the hybridoma culture supernatants for antibodies that bind a given polypeptide, e.g., using a standard ELISA assay.

As an alternative to preparing monoclonal antibody-secreting hybridomas, a monoclonal antibody specific for one of the above described autophagy-related gene products can be identified and isolated by screening a recombinant combinatorial immunoglobulin library (e.g., an antibody phage or yeast display library) with the appropriate autophagy-related gene product to thereby isolate immunoglobulin library members that bind the autophagy-related gene product. Kits for generating and screening phage display libraries are commercially available (e.g., the Pharmacia Recombinant Phage Antibody System, Catalog No. 27-9400-01; and the Stratagene SurfZAP™ Phage Display Kit, Catalog No. 240612), and methods for screening phage and yeast display libraries are known in the art. Examples of methods and reagents particularly amenable for use in generating and screening an antibody display library can be found in, for example, Ladner et al. U.S. Pat. No. 5,223,409; Kang et al. International Publication No. WO 92/18619; Dower et al. International Publication No. WO 91/17271; Winter et al. International Publication WO 92/20791; Markland et al. International Publication No. WO 92/15679; Breitling et al. International Publication WO 93/01288; McCafferty et al. International Publication No. WO 92/01047; Garrard et al. International Publication No. WO 92/09690; Ladner et al. International Publication No. WO 90/02809; Fuchs et al. (1991) Biotechnology (NY) 9:1369-1372; Hay et al. (1992) Hum. Antibod. Hybridomas 3:81-85; Huse et al. (1989) Science 246:1275-1281; Griffiths et al. (1993) EMBO J. 12:725-734; Hawkins et al. (1992) J. Mol. Biol. 226:889-896; Clarkson et al. (1991) Nature 352:624-628; Gram et al. (1992) Proc. Natl. Acad. Sci. USA 89:3576-3580; Garrard et al. (1991) Biotechnology (NY) 9:1373-1377; Hoogenboom et al. (1991) Nucleic Acids Res. 19:4133-4137; Barbas et al. (1991) Proc. Natl. Acad. Sci. USA 88:7978-7982; and McCafferty et al. (1990) Nature 348:552-554.

In addition, chimeric and humanized antibodies against autophagy-related gene products can be made according to standard protocols such as those disclosed in U.S. Pat. No. 5,565,332. In another embodiment, antibody chains or specific binding pair members can be produced by recombination between vectors comprising nucleic acid molecules encoding a fusion of a polypeptide chain of a specific binding pair member and a component of a replicable generic display package and vectors containing nucleic acid molecules encoding a second polypeptide chain of a single binding pair member using techniques known in the art, e.g., as described in U.S. Pat. No. 5,565,332, 5,871,907, or 5,733,743.

In another embodiment, human monoclonal antibodies directed against autophagy-related gene product can be generated using transgenic or transchromosomal mice carrying parts of the human immune system rather than the mouse system. In one embodiment, transgenic mice, referred to herein as “humanized mice,” which contain a human immunoglobulin gene miniloci that encodes unrearranged human heavy and light chain variable region immunoglobulin sequences, together with targeted mutations that inactivate or delete the endogenous μ and κ chain loci (Lonberg, N. et al. (1994) Nature 368(6474): 856 859). The mice may also contain human heavy chain constant region immunoglobulin sequences. Accordingly, the mice express little or no mouse IgM or κ, and in response to immunization, the introduced human heavy and light chain variable region transgenes undergo class switching and somatic mutation to generate high affinity human variable region antibodies (Lonberg, N. et al. (1994), supra; reviewed in Lonberg, N. (1994) Handbook of Experimental Pharmacology 113:49 101; Lonberg, N. and Huszar, D. (1995) Intern. Rev. Immunol. Vol. 13: 65 93, and Harding, F. and Lonberg, N. (1995) Ann. N.Y. Acad. Sci. 764:536 546). These mice can be used to generate fully human monoclonal antibodies using the techniques described above or any other technique known in the art. The preparation of humanized mice is described in Taylor, L. et al. (1992) Nucleic Acids Research 20:6287 6295; Chen, J. et al. (1993) International Immunology 5: 647 656; Tuaillon et al. (1993) Proc. Natl. Acad. Sci. USA 90:3720 3724; Choi et al. (1993) Nature Genetics 4:117 123; Chen, J. et al. (1993) EMBO J. 12: 821 830; Tuaillon et al. (1994) J. Immunol. 152:2912 2920; Lonberg et al., (1994) Nature 368(6474): 856 859; Lonberg, N. (1994) Handbook of Experimental Pharmacology 113:49 101; Taylor, L. et al. (1994) International Immunology 6: 579 591; Lonberg, N. and Huszar, D. (1995) Intern. Rev. Immunol. Vol. 13: 65 93; Harding, F. and Lonberg, N. (1995) Ann. N.Y. Acad. Sci. 764:536 546; Fishwild, D. et al. (1996) Nature Biotechnology 14: 845 851. See further, U.S. Pat. Nos. 5,545,806; 5,569,825; 5,625,126; 5,633,425; 5,789,650; 5,877,397; 5,661,016; 5,814,318; 5,874,299; and 5,770,429; all to Lonberg and Kay, and GenPharm International; U.S. Pat. No. 5,545,807 to Surani et al.

### 6. PHARMACEUTICAL COMPOSITIONS

The invention provides pharmaceutical compositions comprising modulators of autophagy-related gene products. In one aspect, the present invention provides pharmaceutically acceptable compositions which comprise a therapeutically-effective amount of one or more of the agents described above, formulated together with one or more pharmaceutically acceptable carriers (additives) and/or diluents. In another aspect, the agents of the invention can be administered as such, or administered in mixtures with pharmaceutically acceptable carriers and can also be administered in conjunction with other agents. Conjunctive therapy thus includes sequential, simultaneous and separate, or co-administration of one or more agent of the invention, wherein the therapeutic effects of the first administered has not entirely disappeared when the subsequent compound is administered.

As described in detail below, the pharmaceutical compositions of the present invention may be specially formulated for administration in solid or liquid form, including those adapted for the following: (1) oral administration, for example, drenches (aqueous or non-aqueous solutions or suspensions), tablets, e.g., those targeted for buccal, sublingual, and systemic absorption, boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; or (8) nasally.

As set out above, in certain embodiments, agents of the invention may be compounds containing a basic functional group, such as amino or alkylamino, and are, thus, capable of forming pharmaceutically-acceptable salts with pharmaceutically-acceptable acids. These salts can be prepared in situ in the administration vehicle or the dosage form manufacturing process, or through a separate reaction of a purified compound of the invention in its free base form with a suitable organic or inorganic acid, and isolating the salt thus formed during subsequent purification. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like (see, for example, Berge et al. (1977) “Pharmaceutical Salts”, J. Pharm. Sci. 66:1-19).

The pharmaceutically acceptable salts of the subject compounds include the conventional nontoxic salts or quaternary ammonium salts of the compounds, e.g., from non-toxic organic or inorganic acids. For example, such conventional nontoxic salts include those derived from inorganic acids such as hydrochloride, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like.

In other cases, the agents of the present invention may be compounds containing one or more acidic functional groups and, thus, are capable of forming pharmaceutically-acceptable salts with pharmaceutically-acceptable bases. These salts can likewise be prepared in situ in the administration vehicle or the dosage form manufacturing process, or by separately reacting the purified compound in its free acid form with a suitable base, such as the hydroxide, carbonate or bicarbonate of a pharmaceutically-acceptable metal cation, with ammonia, or with a pharmaceutically-acceptable organic primary, secondary or tertiary amine Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like (see, for example, Berge et al., supra).

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

The formulations of the agents of the invention may be presented in unit dosage form and may be prepared by any methods well known in the art of pharmacy. The amount of active ingredient which can be combined with a carrier material to produce a single dosage form will vary depending upon the host being treated and the particular mode of administration. The amount of active ingredient which can be combined with a carrier material to produce a single dosage form will generally be that amount of the agent which produces a therapeutic effect.

In certain embodiments, a formulation of the present invention comprises an excipient, including, but not limited to, cyclodextrins, liposomes, micelle forming agents, e.g., bile acids, and polymeric carriers, e.g., polyesters and polyanhydrides; and an agent of the present invention. In certain embodiments, an aforementioned formulation renders orally bioavailable a agent of the present invention.

Methods of preparing these formulations or compositions may include the step of bringing into association an agent of the present invention with the carrier and, optionally, one or more accessory ingredients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol, glycerol monostearate, and non-ionic surfactants; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-shelled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. Compositions of the invention may also be formulated for rapid release, e.g., freeze-dried. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Formulations of the pharmaceutical compositions of the invention for rectal or vaginal administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations of the present invention which are suitable for vaginal administration also include pessaries, tampons, creams, gels, pastes, foams or spray formulations containing such carriers as are known in the art to be appropriate.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically-acceptable carrier, and with any preservatives, buffers, or propellants which may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the compound in the proper medium. Absorption enhancers can also be used to increase the flux of the compound across the skin. The rate of such flux can be controlled by either providing a rate controlling membrane or dispersing the compound in a polymer matrix or gel.

Ophthalmic formulations, eye ointments, powders, solutions and the like, are also contemplated as being within the scope of this invention.

Pharmaceutical compositions of this invention suitable for parenteral administration comprise one or more compounds of the invention in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

Exemplary formulations comprising agents of the invention are determined based on various properties including, but not limited to, chemical stability at body temperature, functional efficiency time of release, toxicity and optimal dose.

The preparations of the present invention may be given orally, parenterally, topically, or rectally. They are of course given in forms suitable for each administration route. For example, they are administered in tablets or capsule form, by injection, inhalation, eye lotion, ointment, suppository, administration by injection, infusion or inhalation; topical by lotion or ointment; and rectal by suppositories.

Regardless of the route of administration selected, the compounds of the present invention, which may be used in a suitable hydrated form, and/or the pharmaceutical compositions of the present invention, are formulated into pharmaceutically-acceptable dosage forms by conventional methods known to those of skill in the art.

In certain embodiments, the above-described pharmaceutical compositions comprise one or more of the agents of the invention, a chemotherapeutic agent, and optionally a pharmaceutically acceptable carrier.

The term chemotherapeutic agent includes, without limitation, platinum-based agents, such as carboplatin and cisplatin; nitrogen mustard alkylating agents; nitrosourea alkylating agents, such as carmustine (BCNU) and other alkylating agents; antimetabolites, such as methotrexate; purine analog antimetabolites; pyrimidine analog antimetabolites, such as fluorouracil (5-FU) and gemcitabine; hormonal antineoplastics, such as goserelin, leuprolide, and tamoxifen; natural antineoplastics, such as taxanes (e.g., docetaxel and paclitaxel), aldesleukin, interleukin-2, etoposide (VP-16), interferon α, and tretinoin (ATRA); antibiotic natural antineoplastics, such as bleomycin, dactinomycin, daunorubicin, doxorubicin, and mitomycin; and vinca alkaloid natural antineoplastics, such as vinblastine and vincristine.

Further, the following drugs may also be used in combination with a chemotherapetutic agent, even if not considered chemotherapeutic agents themselves: dactinomycin; daunorubicin HCl; docetaxel; doxorubicin HCl; epoetin α; etoposide (VP-16); ganciclovir sodium; gentamicin sulfate; interferon α; leuprolide acetate; meperidine HCl; methadone HCl; ranitidine HCl; vinblastin sulfate; and zidovudine (AZT). For example, fluorouracil has recently been formulated in conjunction with epinephrine and bovine collagen to form a particularly effective combination.

Still further, the following listing of amino acids, peptides, polypeptides, proteins, polysaccharides, and other large molecules may also be used: interleukins 1 through 18, including mutants and analogues; interferons or cytokines, such as interferons α, β, and γ; hormones, such as luteinizing hormone releasing hormone (LHRH) and analogues and, gonadotropin releasing hormone (GnRH); growth factors, such as transforming growth factor-β (TGF-β), fibroblast growth factor (FGF), nerve growth factor (NGF), growth hormone releasing factor (GHRF), epidermal growth factor (EGF), fibroblast growth factor homologous factor (FGFHF), hepatocyte growth factor (HGF), and insulin growth factor (IGF); tumor necrosis factor-α & β (TNF-α & β); invasion inhibiting factor-2 (IIF-2); bone morphogenetic proteins 1-7 (BMP 1-7); somatostatin; thymosin-α-1; γ-globulin; superoxide dismutase (SOD); complement factors; anti-angiogenesis factors; antigenic materials; and pro-drugs.

Chemotherapeutic agents for use with the compositions and methods of treatment described herein include, but are not limited to alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammalI and calicheamicin omegal1; dynemicin, including dynemicin A; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, caminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., paclitaxel and doxetaxel; chlorambucil; gemcitabine; 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; vinorelbine; novantrone; teniposide; edatrexate; daunomycin; aminopterin; XELODA® (capecitabine); ibandronate; irinotecan (e.g., CPT-11); topoisomerase inhibitor RFS 2000; difluoromethylomithine (DMFO); retinoids such as retinoic acid; capecitabine; and pharmaceutically acceptable salts, acids or derivatives of any of the above.

In another embodiment, the composition of the invention may comprise other biologically active substances, including therapeutic drugs or pro-drugs, for example, other chemotherapeutic agents, scavenger compounds, antibiotics, anti-virals, anti-fungals, anti-inflammatories, vasoconstrictors and anticoagulants, antigens useful for cancer vaccine applications or corresponding pro-drugs.

Exemplary scavenger compounds include, but are not limited to thiol-containing compounds such as glutathione, thiourea, and cysteine; alcohols such as mannitol, substituted phenols; quinones, substituted phenols, aryl amines and nitro compounds.

Various forms of the chemotherapeutic agents and/or other biologically active agents may be used. These include, without limitation, such forms as uncharged molecules, molecular complexes, salts, ethers, esters, amides, and the like, which are biologically active.

### 7. THERAPEUTIC METHODS OF THE INVENTION

The present invention further provides novel therapeutic methods of treating autophagy-related diseases, including cancer, neurodegenerative diseases, liver diseases, muscle diseases and pancreatitis, comprising administering to a subject, (e.g., a subject in need thereof), an effective amount of a modulator of an autophagy-related gene product of the invention.

A subject in need thereof may include, for example, a subject who has been diagnosed with a tumor, including a pre-cancerous tumor, a cancer, or a subject who has been treated, including subjects that have been refractory to previous treatment.

Autophagy has been implicated as playing a role in the axonal degeneration that occurs following nerve injury. For example, traumatic spinal cord injury results in a rapid increase of itraaxonal calcium levels, which results in an increase in neuronal autophagy and cell death (Knoferle et al., (2009), PNAS, 107, 6064-6069). Inhibition of either calcium flux or autophagy attenuates axonal degeneration. Notably, a number of calcium binding proteins were identified in the autophagy modulator screen of the instant invention (Table 5). Thus, in certain embodiments the invention relates to the treatment or prevention of axonal degeneration following neural trauma through the modulation of calcium-binding autophagy modulating gene products or through the modulation of other autophagy-related gene products.

The methods of the present invention may be used to treat any cancerous or pre-cancerous tumor. Cancers that may treated by methods and compositions of the invention include, but are not limited to, cancer cells from the bladder, blood, bone, bone marrow, brain, breast, colon, esophagus, gastrointestine, gum, head, kidney, liver, lung, nasopharynx, neck, ovary, prostate, skin, stomach, testis, tongue, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; and roblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malig melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's lymphoma; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

In certain embodiments, the methods of the present invention include the treatment of cancer comprising the administration of an autophagy-inhibiting agent of the present invention in combination with a chemotherapeutic agent. Such autophagy-inhibiting agents include agents that inhibit the activity of products of autophagy-enhancing genes (Table 2) and agents that enhance the activity of the products of autophagy-inhibiting genes (Table 1). Any chemotherapeutic agent is suitable for use in the methods of the instant invention, particularly chemotherapeutic agents that that induce cellular stress in cancer cells. Chemotherapeutic agents useful in the instant invention include, but are not limited to, to alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammalI and calicheamicin omegal1; dynemicin, including dynemicin A; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, caminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., paclitaxel and doxetaxel; chlorambucil; gemcitabine; 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; vinorelbine; novantrone; teniposide; edatrexate; daunomycin; aminopterin; XELODA® (capecitabine); ibandronate; irinotecan (e.g., CPT-11); topoisomerase inhibitor RFS 2000; difluoromethylomithine (DMFO); retinoids such as retinoic acid; capecitabine; and pharmaceutically acceptable salts, acids or derivatives of any of the above.

In certain embodiments, the methods of the present invention include the treatment of cancer comprising the administration of an autophagy-inhibiting agent of the present invention in combination with radiation therapy. An optimized dose of radiation therapy may be given to a subject as a daily dose. Optimized daily doses of radiation therapy may be, for example, from about 0.25 to 0.5 Gy, about 0.5 to 1.0 Gy, about 1.0 to 1.5 Gy, about 1.5 to 2.0 Gy, about 2.0 to 2.5 Gy, and about 2.5 to 3.0 Gy. An exemplary daily dose may be, for example, from about 2.0 to 3.0 Gy. A higher dose of radiation may be administered, for example, if a tumor is resistant to lower doses of radiation. High doses of radiation may reach, for example, 4 Gy. Further, the total dose of radiation administered over the course of treatment may, for example, range from about 50 to 200 Gy. In an exemplary embodiment, the total dose of radiation administered over the course of treatment ranges, for example, from about 50 to 80 Gy. In certain embodiments, a dose of radiation may be given over a time interval of, for example, 1, 2, 3, 4, or 5 minutes, wherein the amount of time is dependent on the dose rate of the radiation source.

In certain embodiments, a daily dose of optimized radiation may be administered, for example, 4 or 5 days a week, for approximately 4 to 8 weeks. In an alternate embodiment, a daily dose of optimized radiation may be administered daily seven days a week, for approximately 4 to 8 weeks. In certain embodiments, a daily dose of radiation may be given a single dose. Alternately, a daily dose of radiation may given as a plurality of doses. In a further embodiment, the optimized dose of radiation may be a higher dose of radiation than can be tolerated by the patient on a daily base. As such, high doses of radiation may be administered to a patient, but in a less frequent dosing regimen.

The types of radiation that may be used in cancer treatment are well known in the art and include electron beams, high-energy photons from a linear accelerator or from radioactive sources such as cobalt or cesium, protons, and neutrons. An exemplary ionizing radiation is an x-ray radiation.

Methods to administer radiation are well known in the art. Exemplary methods include, but are not limited to, external beam radiation, internal beam radiation, and radiopharmaceuticals. In external beam radiation, a linear accelerator is used to deliver high-energy x-rays to the area of the body affected by cancer. Since the source of radiation originates outside of the body, external beam radiation can be used to treat large areas of the body with a uniform dose of radiation. Internal radiation therapy, also known as brachytherapy, involves delivery of a high dose of radiation to a specific site in the body. The two main types of internal radiation therapy include interstitial radiation, wherein a source of radiation is placed in the effected tissue, and intracavity radiation, wherein the source of radiation is placed in an internal body cavity a short distance from the affected area. Radioactive material may also be delivered to tumor cells by attachment to tumor-specific antibodies. The radioactive material used in internal radiation therapy is typically contained in a small capsule, pellet, wire, tube, or implant. In contrast, radiopharmaceuticals are unsealed sources of radiation that may be given orally, intravenously or directly into a body cavity.

Radiation therapy may also include sterotactic surgery or sterotactic radiation therapy, wherein a precise amount of radiation can be delivered to a small tumor area using a linear accelerator or gamma knife and three dimensional conformal radiation therapy (3DCRT), which is a computer assisted therapy to map the location of the tumor prior to radiation treatment.

A subject in need thereof may also include, for example, a subject who has been diagnosed with a neurodegenerative disease or a subject who has been treated for a neurodegenerative disease, including subjects that have been refractory to the previous treatment.

The methods of the present invention may be used to treat any neurodegenerative disease. In certain embodiments, the neurodegenerative disease is a proteinopathy, or protein-folding disease. Examples of such proteinopathies include, but are not limited to, Alzheimer's disease, Parkinson's disease, Lewy Body Dementia, ALS, Huntington's disease, spinocerebellar ataxias and spinobulbar musclular atrophy. In other embodiments, the methods of the present invention can be used to treat any neurodegenerative disease. Neurodegenerative diseases treatable by the methods of the present invention include, but are not limited to, Adrenal Leukodystrophy, alcoholism, Alexander's disease, Alper's disease, Alzheimer's disease, Amyotrophic lateral sclerosis, ataxia telangiectasia, Batten disease, bovine spongiform encephalopathy, Canavan disease, cerebral palsy, cockayne syndrome, corticobasal degeneration, Creutzfeldt-Jakob disease, familial fatal insomnia, frontotemporal lobar degeneration, Huntington's disease, HIV-associated dementia, Kennedy's disease, Krabbe's disease, Lewy body dementia, neuroborreliosis, Machado-Joseph disease, multiple system atrophy, multiple sclerosis, narcolepsy, Niemann Pick disease, Parkinson's disease, Pelizaeus-Merzbacher disease, Pick's disease, primary lateral sclerosis, prion diseases, progressive supranuclear palsy, Refsum's disease, Sandhoff disease, Schilder's disease, subacute combined degeneration of spinal cord secondary to pernicious anaemia, Spielmeyer-Vogt-Sjogren-Batten disease, spinocerebellar ataxia, spinal muscular atrophy, Steele-Richardson-Olszewski disease, Tabes dorsalis and toxic encephalopathy.

A subject in need thereof may also include, for example, a subject who has been diagnosed with a liver disease or a subject who has been treated for a liver disease, including subjects that have been refractory to previous treatment. In certain embodiments, the liver disease is a proteinopathy, or protein-folding disease. An example of such a proteinopathy is α1-antitrypsin deficiency.

A subject in need thereof may also include, for example, a subject who has been diagnosed with a muscle disease or a subject who has been treated for a muscle disease, including subjects that have been refractory to previous treatment. In certain embodiments, the muscle disease is a proteinopathy, or protein-folding disease. Examples of such a proteinopathies include, but are not limited to, deficiency sporadic inclusion body myositis, limb girdle muscular dystrophy type 2B and Miyoshi myopathy.

A subject in need thereof may also include, for example, a subject who has been diagnosed with a proteinopathy, including subjects that have been refractory to previous treatment. Examples of proteinopathies include, but are not limited to Alzheimer's disease, cerebral β-amyloid angiopathy, retinal ganglion cell degeneration, prion diseases (e.g. bovine spongiform encephalopathy, kuru, Creutzfeldt-Jakob disease, variant Creutzfeldt-Jakob disease, Gerstmann-Straussler-Scheinker syndrome, fatal familial insomnia) tauopathies (e.g. frontotemporal dementia, Alzheimer's disease, progressive supranuclear palsy, corticobasal degeration, frontotemporal lobar degeneration), frontemporal lobar degeneration, amyotrophic lateral sclerosis, Huntington's disease, familial British dementia, Familial Danish dementia, hereditary cerebral hemorrhage with amyloidosis (Iclandic), CADASIL, Alexander disease, Seipinopathies, familial amyloidotic neuropothy, senile systemic amyloidosis, serpinopathies, AL amyloidosis, AA amyloidosis, type II diabetes, aortic medial amyloidosis, ApoAI amyloidosis, ApoII amyloidosis, ApoAIV amyloidosis, familial amyloidosis of the Finish type, lysozyme amyloidosis, fibrinogen amyloidosis, dialysis amyloidosis, inclusion body myositis/myopathy, cataracts, medullary thyroid carcinoma, cardiac atrial amyloidosis, pituitary prolactinoma, hereditary lattice corneal dystrophy, cutaneous lichen amyloidosis, corneal lactoferrin amyloidosis, corneal lactoferrin amyloidosis, pulmonary alveolar proteinosis, odontogenic tumor amylois, seminal vesical amyloid, cystric fibrosis, sickle cell disease and critical illness myopathy.

In some embodiments, the subject pharmaceutical compositions of the present invention will incorporate the substance or substances to be delivered in an amount sufficient to deliver to a patient a therapeutically effective amount of an incorporated therapeutic agent or other material as part of a prophylactic or therapeutic treatment. The desired concentration of the active agent will depend on absorption, inactivation, and excretion rates of the drug as well as the delivery rate of the compound. It is to be noted that dosage values may also vary with the severity of the condition to be alleviated. It is to be further understood that for any particular subject, specific dosage regimens should be adjusted over time according to the individual need and the professional judgment of the person administering or supervising the administration of the compositions. Typically, dosing will be determined using techniques known to one skilled in the art.

The dosage of the subject agent may be determined by reference to the plasma concentrations of the agent. For example, the maximum plasma concentration (Cmax) and the area under the plasma concentration-time curve from time 0 to infinity (AUC (0-4)) may be used. Dosages for the present invention include those that produce the above values for Cmax and AUC (0-4) and other dosages resulting in larger or smaller values for those parameters. Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular agent employed, the route of administration, the time of administration, the rate of excretion or metabolism of the particular compound being employed, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could prescribe and/or administer doses of the agents of the invention employed in the pharmaceutical composition at levels lower than that required in order to achieve the desired therapeutic effect and gradually increase the dosage until the desired effect is achieved.

In general, a suitable daily dose of an agent of the invention will be that amount of the agent which is the lowest dose effective to produce a therapeutic effect. Such an effective dose will generally depend upon the factors described above.

If desired, the effective daily dose of the agent may be administered as two, three, four, five, six or more sub-doses administered separately at appropriate intervals throughout the day, optionally, in unit dosage forms.

The precise time of administration and amount of any particular agent that will yield the most effective treatment in a given patient will depend upon the activity, pharmacokinetics, and bioavailability of a particular agent, physiological condition of the patient (including age, sex, disease type and stage, general physical condition, responsiveness to a given dosage and type of medication), route of administration, and the like. The guidelines presented herein may be used to optimize the treatment, e.g., determining the optimum time and/or amount of administration, which will require no more than routine experimentation consisting of monitoring the subject and adjusting the dosage and/or timing.

While the subject is being treated, the health of the subject may be monitored by measuring one or more of the relevant indices at predetermined times during a 24-hour period. All aspects of the treatment, including supplements, amounts, times of administration and formulation, may be optimized according to the results of such monitoring. The patient may be periodically reevaluated to determine the extent of improvement by measuring the same parameters, the first such reevaluation typically occurring at the end of four weeks from the onset of therapy, and subsequent reevaluations occurring every four to eight weeks during therapy and then every three months thereafter. Therapy may continue for several months or even years, with a minimum of one month being a typical length of therapy for humans. Adjustments, for example, to the amount(s) of agent administered and to the time of administration may be made based on these reevaluations.

Treatment may be initiated with smaller dosages which are less than the optimum dose of the compound. Thereafter, the dosage may be increased by small increments until the optimum therapeutic effect is attained. In addition, the combined use an agent that modulates a autotrophy-associated gene product and a second agent, e.g. another agent useful for the treatment of the autophagy-related disease, may reduce the required dosage for any individual agent because the onset and duration of effect of the different compounds and/or agents may be complimentary.

### EXAMPLES

### Materials and Methods

### Cell Lines and Culture Conditions

H4 human neuroblastoma cells were cultured under standard tissue culture conditions in DMEM media supplemented with 10% normal calf serum, penicillin/streptomycin, sodium pyruvate (Invitrogen) and, where appropriate, 0.4-1.2 mg/mL G418. LC3-GFP and FYVE-dsRed H4 cells were generated as described in Zhang et al., PNAS, 102, 15545-15550 (2007). To create a stable line expressing Lamp1, H4 cells were transfected with Lamp1-RFP plasmid using TRANSIT_LT1® transfection reagent (Mirus), followed by selection with 0.4 mg/mL G418. Bcl-2 expressing cell lines were created by infecting LC3-GFP and FYVE-dsRed H4 cells with pBabe-Bcl-2 retrovirus, followed by selection with 1 μg/mL puromycin.

For the cytokine assays, cells were seeded at 0.5×10⁵ in full medium in either 24-well (western) or 96-well (LC3-GFP quantification) plates. After 24 hours, cells were washed in PBS and serum-free OPTIMEM™ medium (Invitrogen) was added along with the indicated growth factors and/or cytokines for an additional 24 hours. Growth factors and cytokines used include human TNFα (Cell Sciences), human LIF (GeneScript Corporation), human FGF2 (ProSpec), human IGF1 (ProSpec), human SDF1 (Prospec) and human CLCF1 (R&D Systems). To induce starvation, cells were cultured for 24 hours in full media, washed in PBS and cultured for additional 4 hours in HBSS media (Invitrogen). Where indicated, 2.5 mM N-acetyl-L-cysteine (NAC, Sigma) was added at the time of media change.

For antioxidant assays, cells were treated 24 hours after siRNA transfection with N-acetyl-L-cysteine (NAC, Sigma) at a concentration of 2.5 mM and cultured for additional 48 hours before fixation and image analysis (see below for details). For western blot analysis, lysosomal protease inhibitor E64d (Sigma) was added at a concentration of 10 μg/mL for the last 8-12 hours before cell lysis.

siRNA Transfection

For the primary screens, an arrayed library of 21,121 siRNA pools covering the majority of the human genome were used (Dharmacon siARRAY™ siRNA library (Human Genome, G-005000-05), Thermo Fisher Scientific, Lafayette, Colo.). Each pool contained of 4 unique oligonucleotides targeting different sequences from the same gene. Each assay plate also included the following controls: non-targeting siRNA, mTOR siRNA, ATG5 siRNA and PLK1 siRNA (a transfection efficiency control). siRNAs were transiently transfected in triplicate into H4 cells stably expressing a LC3-GFP reporter at a final concentration of 40 nM using reverse transfection with the HIPERFECT® reagent (Qiagen). HIPERFECT® reagent was diluted 1:20 in DMEM and 8 μl of the mixture was added to wells of 384 well plates. The plates were centrifuged at 1,000 rpm, after which 2 μl of 1 μM arrayed siRNA pools were added to each well. After 30 minutes of incubation, 500 cells in 40 μl of media were added to the wells. Cells were incubated for 72 hours under standard culture conditions, counterstained with 0.5 μM Hoechst 33342 (Invitrogen) for 1 hour and fixed by addition of 30 μl of 8% paraformaldehyde. After 30 minutes, cells were washed 3 times with PBS prior to analysis.

For secondary screens, a siRNA library was used in which the 4 siRNAs of each siRNA pool were separated into individual wells. The cells were transfected and treated as in the primary screen, except that siRNAs were used at a final concentration of 30 nM (1.5 μL/well of 1 uM stock) and HIPERFECT® reagent was diluted 1:30 in OPTIMEM™ (Invitrogen). The secondary screen transfections were done in 2 rounds: in the first one a 1:1 mixture of H4 cells stably expressing LC3-GFP with FYVE-dsRed was transfected in triplicate; in the second round a 1:1 mixture of H4 cells expressing LC3-GFP with Lamp1-RFP was transfected in duplicate. All tertiary characterization screens were done in duplicate using a mixture of LC3-GFP and FYVE-dsRed cells. Each assay plate included 10-12 wells of non-targeting siRNA as well as mTOR, ATG5, PLK1 and, depending on screen, Vps34 or SOD1 siRNA controls.

For low-throughput confirmation of screen hits, cells were transfected in 12- or E-well plates using reverse transfection with 2 μl or 6 μl of HIPERFECT® reagent per mL of media, 40 nM or 10 nM final siRNA concentration and cells at 5×10⁴ or 2×10⁵ cells/mL for H4 and MCF7 cells, respectively. For RT-PCR and FACS analysis, cells were harvested after 72 hours. For western and imaging analysis, cells were split 24 hours after transfection into 24-well plates at 2.5×10⁴ or 1×10⁵ cells/ml and harvested after additional 48 hours.

### Imaging and Image Quantification

For high-throughput screens, cells were imaged on an automated CellWoRx microscope (Applied Precision) at 10× magnification using 2 wavelengths (350 nm to detect Hoechst, 488 nm to detect LC3-GFP) for the primary screens and 3 wavelengths (350 nm, 488 nm and 550 nm to detect Lamp1-RFP or FYVE-dsRed) for the secondary screens. All images were quantified using VHSscan and VHSview image analysis software (Cellomics). Total cell number, total LC3-GFP intensity/cell as well as number, area and intensity of LC3-GFP positive autophagosomes/cell were scored. All dead and mitotic cells were excluded from analysis based on nuclear intensity. The final autophagy score for each well was obtained by multiplying the total autophagosome intensity/cell by the number of autophagosomes/cell and dividing by the average cell intensity. This formula was empirically determined to accurately measure LC3-GFP translocation from cytosol into autophagosomes as reflected by consistently significant z-scores and p-values when using siRNAs against mTOR and Atg5 controls. FYVE-dsRed and Lamp1-RFP scores were obtained in a manner similar to LC3-GFP scores, except that for Lamp1-RFP, which measures total accumulation of the reporter rather than its translocation, division by the average cell intensity was omitted.

For low-throughput follow-up analysis, cells were grown on glass cover slips. Following fixation in 4% paraformaldehyde and counterstaining with Hoechst, cover slips were mounted in 50% glycerol, 0.1% n-propyl gallate/PBS. Cells were imaged at 40× magnification on a Nikon Eclipse E800 microscope. Cell numbers, cell area and intensity, as well as autophagosome number and intensity, were quantified using Metamorph software. Autophagy was scored as number of autophagosomes per cell.

### In-Cell-Western Assays

For quantitative analysis of mTORC1 signaling and induction of endoplasmic reticulum stress, in-cell-western analysis of rpS6 phosphorylation and KDEL (GRP78/GRP94) expression (‘KDEL’ disclosed as SEQ ID NO: 2), respectively, were performed. H4 cells were cultured in 384-well plates and fixed and counterstained as described for the LC3-GFP assay. Following imaging, the cells were permeabilized in PBS containing 0.2% Tx-100 and stained with ALEXA FLOUR®_{—}680NHS-ester, a non-specific lysine reactive probe used to measure relative cell number, at 20 ng/mL for 15 minutes. Subsequently, the cells were washed with PBS containing 0.2% Tx-100 and incubated for 30 minutes in blocking buffer (LICOR® Blocking Buffer diluted 1:1 with PBS+0.2% Tx-100). Cells were then incubated overnight with a rabbit-anti-rpS6 phospho-235/236 (Cell Signaling Technologies), or mouse-anti-KDEL (Stressgen) antibody (‘KDEL’ disclosed as SEQ ID NO: 2) diluted 1:1000 in blocking buffer. Following primary antibody staining, the cells were washed in PBS+0.2% Tx-100 and stained with an IRDye-800-conjugated secondary antibody (LICOR®) diluted 1:1000 in blocking buffer. The plates were scanned on the Aerius infrared imaging system (LICOR®). The intensities of both, the rpS6 phospho-235/236 or KDEL staining (‘KDEL’ disclosed as SEQ ID NO: 2), and of NHS-ester staining were integrated, and the normalized phospho-S6 or KDEL score (‘KDEL’ disclosed as SEQ ID NO: 2) were calculated by dividing phospho-rpS6 or KDEL intensity (‘KDEL’ disclosed as SEQ ID NO: 2) by NHS-ester intensity.

### Statistical Analysis

All screen data was normalized by conversion to logarithmic scale (log 10). For primary screens, z-scores were calculated based on plate median (controls excluded) and Median Absolute Deviation (MAD), with z-score=(cell score−median plate score)/(plate MAD X1.4826). The screen hits were than selected based on the median z-score of the 3 replica-plates with cutoffs set at z-score>1.7 or <−1.9, which gives a p value of 0.02. The same method was used for the rpS6 and KDEL secondary screens (‘KDEL’ disclosed as SEQ ID NO: 2) except the assays were performed in duplicate. For LC3-GFP, FYVE-dsRed and Lamp1-RFP secondary screens z-scores were calculated based on non-targeting siRNA control mean and standard deviation. For secondary confirmation of hits in the LC3-GFP assay it was required that at least 2 out of 4 individual siRNA oligonucleotides for each gene had median z-scores>1.5 or <−1.5 based on 5 replica plates and were consistent with the primary screen z-score. This resulted in p<0.01. In all other secondary assays z-scores>1.5 and <−1.5 were also considered significant. The final z-scores for confirmed genes were calculated based on average z-scores of all wells for oligonucleotides considered positive in the secondary LC3-GFP assay.

The correlation analysis between LC3-GFP and other secondary assays was performed based on individual assay well quadrant analysis: for each well a score of +1 was assigned if z-scores for both features were >1.5 or both were <−1.5; a score of −1 if one z-score was >1.5 while the other was <−1.5; a score of 0 if either z-score failed to reach the cut-off. The individual well scores were than summed up for each gene for all oligonucleotides considered significant in the LC3-GFP secondary assay and divided by the total number of wells assayed for these oligonucleotides. A correlation between features was considered to be positive if the final score was ≧0.5, negative if it was ≦−0.5.

Relative viability was calculated by dividing number of cells in each well based on Hoechst imaging by the average cell number in the plate. The reported viability for each hit gene reflects average viability of all wells for oligonucleotides positive in the secondary LC3-GFP assay. The number of positive oligonucleotides with average viability below 50% is also reported. The relative viability for +NAC and Bcl-2 tertiary assays was calculated by dividing number of cells in each well by the average cell numbers in matching control plates without NAC or Bcl-2, respectively.

Unless otherwise indicated, all remaining p values were calculated from a 2-tailed student t-test with equal variance. All error bars are standard error.

### Western Analysis

For western blots, cells were lysed in Lammeli sample buffer, resolved on a 10-12% SDS-PAGE gel and transferred to PVDF membrane. The following antibodies were used: LC3 (Novus), p62 (Pharmigen), phospho-S6K (Thr389), phospho-Akt (Ser473), phospho-Stat3 (Tyr705), RelA, Sod1, phospho-PTEN (Ser380/Thr382/383) (all Cell Signaling), Bcl-2 (Santa Cruz), all at 1:1000, phospho-S6 (Ser235/236) (Cell Signaling) and phospho-ERK 1/2 (Sigma) at 1:2000, tubulin (Sigma) at 1:5000. Where indicated, blots were quantified using NIH ImageJ64 software.

### Semi-Quantitative RT-PCR

Total RNA was prepared using RNEASY® miniprep kits (Qiagen) according to the manufacturer's instructions. For cDNA synthesis, 1.25 μg of RNA was used in the SUPERSCRIPT® First-Strand Synthesis System for RT-PCR (Invitrogen) with oligo dT primers. The following primers were used in the RT-PCR reactions: RelA AGCGCATCCAGACCAACAACAACC (SEQ ID NO: 3) and CCGCCGCAGCTGCATGGAGACC (SEQ ID NO: 4), AMPKα2 CACCTCGCCTGGGCAGTCACACC (SEQ ID NO: 5) and ATTGGGGGCATAAACACAGCATAA (SEQ ID NO: 6), Sod1 GGTGCTGGTTTGCGTCGTAGTCTC (SEQ ID NO: 7) and ACCAGTGTGCGGCCAATGATG (SEQ ID NO: 8), β actin GACCTGACAGACTACCTCAT (SEQ ID NO: 9) and AGACAGCACTGTGTTGGCTA (SEQ ID NO: 10). PCR product was resolved on 2% agarose gels and quantified using NIH ImageJ64 software.

### Quantification of Cellular Reactive Oxygen Species (ROS) Levels

ROS levels were quantified 72 hours after siRNA transfection using IMAGE-IT® LIVE Green ROS Detection Kit for microscopy (Molecular Probes) according to the manufacturer's instructions. Images were acquired on a Nikon Eclipse E800 microscope at 40× magnification and quantified using Metamorph software. Alternatively, ROS levels were quantified following 4 hour starvation in HBSS. Cells were stained with 10 μM dihydroethidium for 20 min at 37° C., washed twice in PBS and analyzed by flow cytometry.

### Bioinformatics Analysis

For enrichment analyses, siRNA screen hit genes were classified into functional categories such as biological process, molecular function (PANTHER classification system), cellular component (Gene Ontology (GO) classification system), canonical pathways (MSigDB) and transcription factor binding sites (MSigDB and TRANSFAC v7.4). To assess the statistical enrichment or over-representation of these categories for the hit genes relative to their representation in the global set of genes examined in the siRNA screen, P-values were computed using the hypergeometric probability distribution, which was implemented in the R language.

For the protein interaction network, the network was constructed by iteratively connecting interacting proteins, with data extracted from genome-wide interactome screens, from databases: HPRD, MINT, REACTOME and curated literature entries. For yeast interaction data, yeast proteins were mapped to human orthologs (reciprocal Blastp analysis and Homologene). The network uses graph theoretic representations, which abstract components (gene products) as nodes and relationships (interactions) between components as edges, implemented in the Perl programming language.

### Analysis of Hit Gene Expression During Aging

Gene expression during aging analysis was based on Affymetrix HG-U1331_Plus_{—}2 microarray data of young (≦40 years old) and old (≧70 years old) human brain samples. Array normalization, expression value calculation and clustering analysis were performed using the dChip software. Hierarchical clustering analysis was used to group genes or samples with similar expression pattern. Two genes or samples with the closest distance were first merged into a super-gene or super-sample and connected by branches with length representing their distance, and were deleted from future merging. Then the next pair of genes or samples (super-genes or super-samples) with the smallest distance was than chosen to be merged. The process was repeated until all the genes and samples were merged into one cluster.

### Example 1

### A High-Throughput Image-Based siRNA Screen for Genes Involved in the Regulation of Autophagy

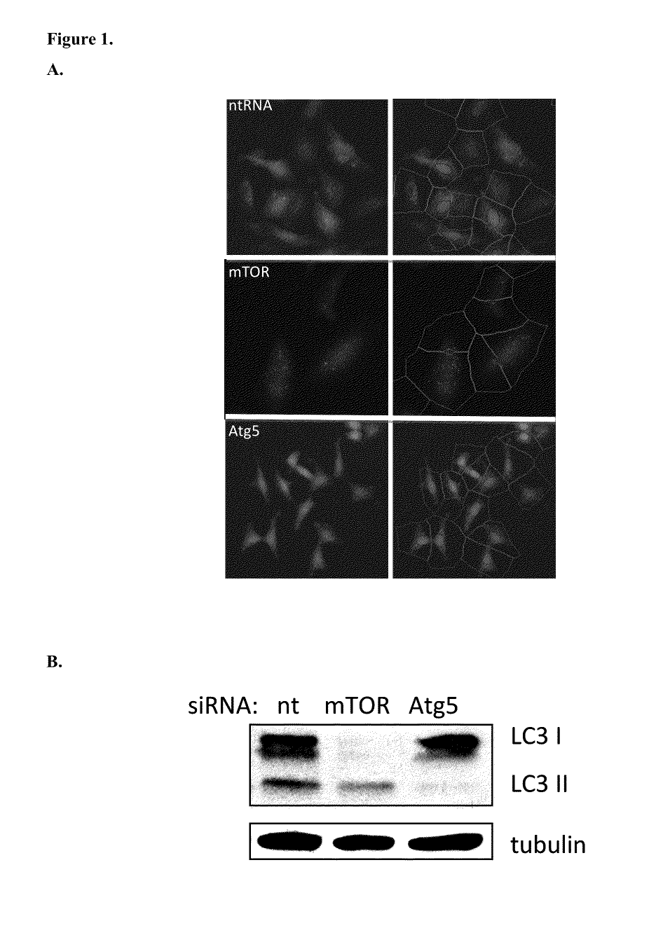

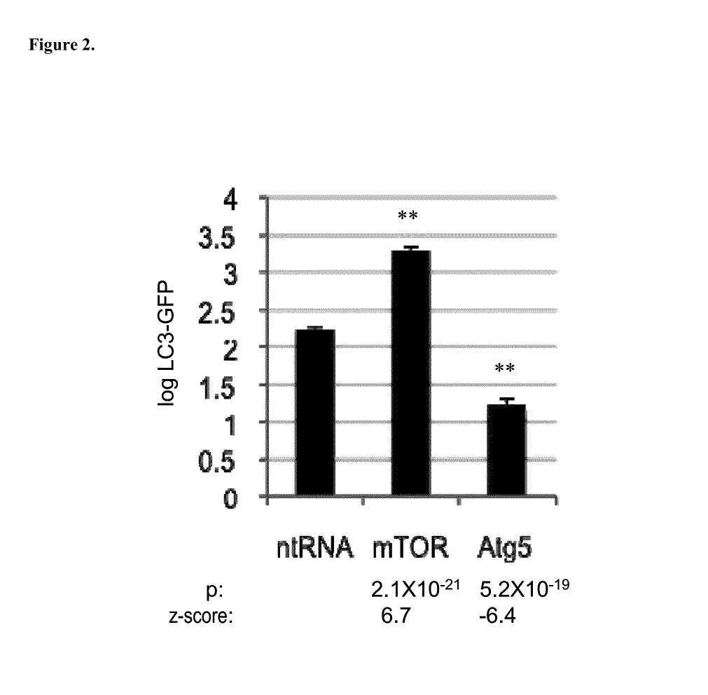

Human neuroblastoma H4 cells stably expressing the LC3-GFP reporter were used to identify genes involved in the regulation of autophagy in mammals. Under normal growth conditions, LC3-GFP in these cells exhibits a diffused cytosolic localization. When autophagy is induced in these cells, LC3-GFP is recruited from the cytosol and can be visualized in a punctate pattern corresponding to autophagosomes. In order to validate the system, cells were transfected with siRNA against either the essential autophagy mediator ATG5 or against mTOR, a suppressor of starvation-induced autophagy. Following 72 hours of incubation under normal nutritional conditions, cells were transfected with ATG5 siRNA. This led to significant down-regulation of autophagy as assessed by a reduction in the number and intensity of LC3-GFP positive autophagosomes (FIG. 1A), as well as a decrease in LC3II to LC3I ratio on a western blot (FIG. 1B). Conversely, expression of siRNA against mTOR, the catalytic subunit of mTORC1, led to an increase in the number and intensity of LC3-GFP positive autophagosomes (FIG. 1A) and an increase in LC3II to LC3I ratio (FIG. 1B). Quantification of the LC3-GFP images in 384-well format acquired on a high-throughput automated fluorescent microscope revealed that the changes in the levels of autophagy following ATG5 or mTOR siRNA transfection were statistically significant as compared to non-targeting, control siRNA (FIG. 2).

This system was used to screen a human genome siRNA library containing siRNA pools targeting 21,121 genes, with each pool containing 4 independent siRNA oligonucleotides for each gene. The primary screen was performed in triplicate and resulted in the identification of 574 genes (2.7% of the all genes tested) which knock-down led to a median decrease in LC3-GFP positive autophagosome formation by at least 1.9 standard deviations (SD) or increase by at least 1.7 SD from the plate median.

The candidate genes identified in the primary screen were confirmed using a deconvolved library, in which the 4 siRNAs from each pool were evaluated separately. Of the 547 candidate genes, 236 (41%) were confirmed with at least 2 independent siRNA oligonucleotides resulting in median increase or decrease in the levels of autophagy by at least 1.5 SD as compared to non-targeting siRNA control (FIG. 3, p<0.05). Knock-down of a majority of these hits (219, 93% of all confirmed genes, Table 1) led to the induction of autophagy, indicating that these genes were autophagy-inhibiting genes, while knockdown of the remaining 17 hits led to the inhibition of autophagy, indicating that these genes were autophagy-enhancing genes (Table 2).

### Example 2

### A Secondary High-Throughput Characterization of the Candidate Genes

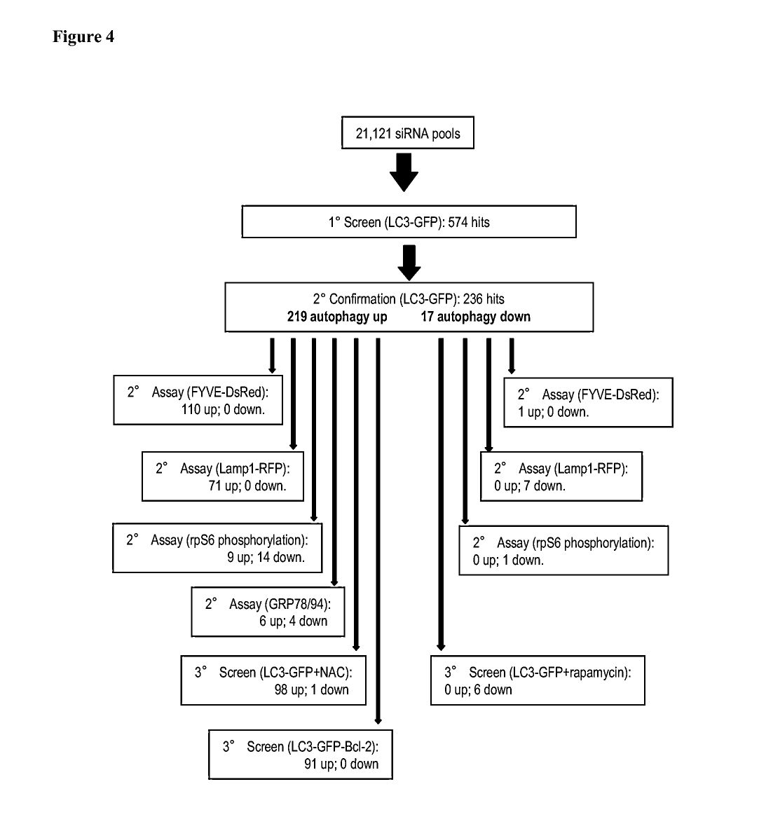

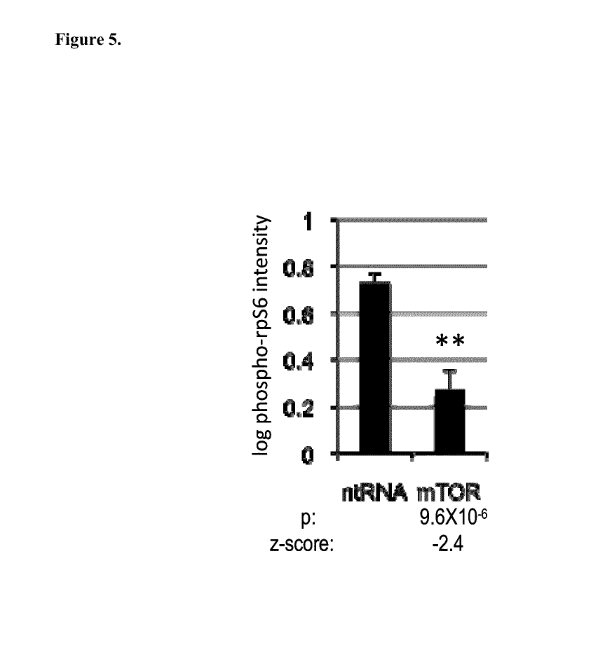

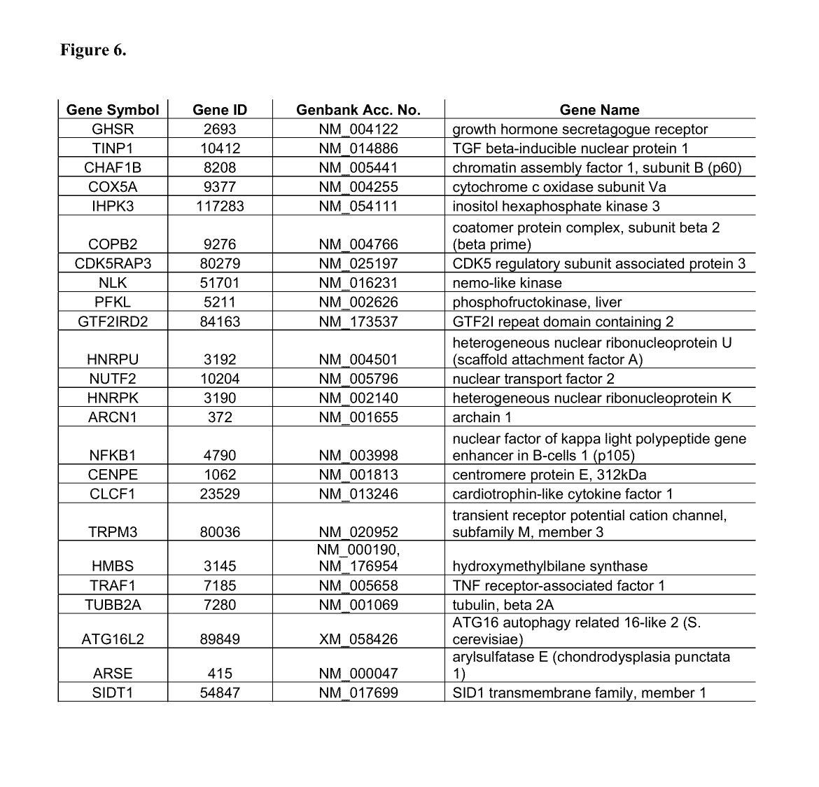

In order to elucidate the molecular pathways involved in regulation of autophagy by the newly identified genes, additional high-throughput assays were developed and performed to characterize the hits (FIG. 4). In one of these assays, the function of mTORC1, an essential mediator of starvation-induced autophagy was investigated. To determine which of the candidate genes regulate autophagy by altering mTORC1 activity, an in-cell-western assay was used to evaluate the phosphorylation status of a downstream target of mTORC1 signaling, the ribosomal S6 protein (rpS6). To validate this system, H4 cells were transfected with mTOR siRNA. A significant decrease in the levels of rpS6 phosphorylation in mTOR siRNA transfected cells as compared to non-targeting siRNA was observed (FIG. 5). Using the in-cell-western assay it was determined that only 14 (6%) out of the 219 confirmed genes which knockdown led to the induction of autophagy were strongly correlated with down-regulation of mTORC1 activity, while nine genes (4%) were identified in which knockdown led to up-regulation of both autophagy and of mTORC1 activity (FIG. 6).

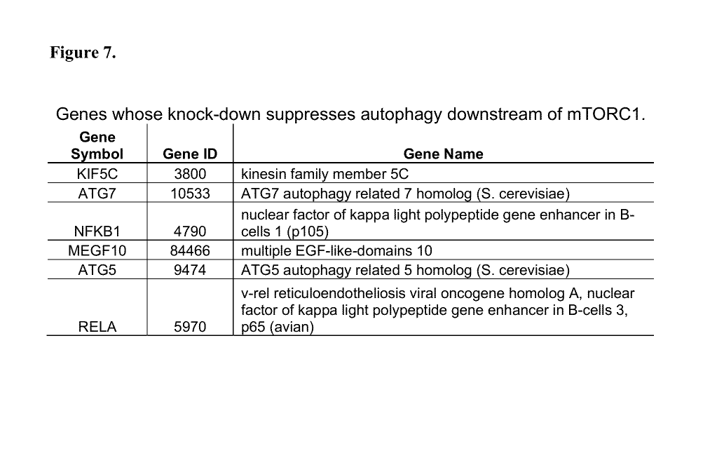

In a follow up tertiary screen of the 17 confirmed genes which knock down resulted in suppression of autophagy, 35% of these genes were found to be able to down-regulate autophagy in the presence of rapamycin, a potent inhibitor of mTORC1, which indicates that such genes function downstream of mTORC1 (FIG. 7).

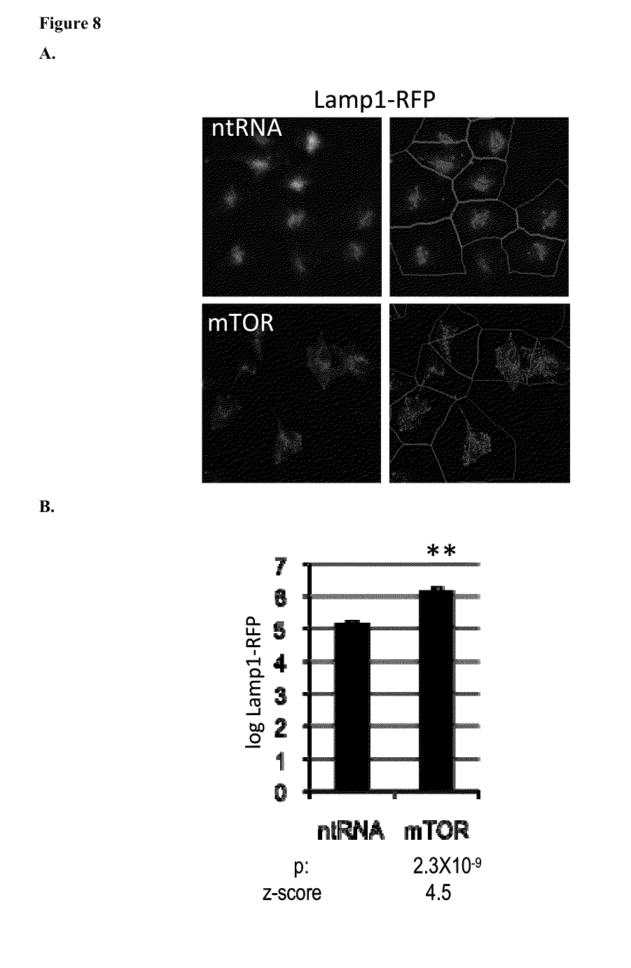

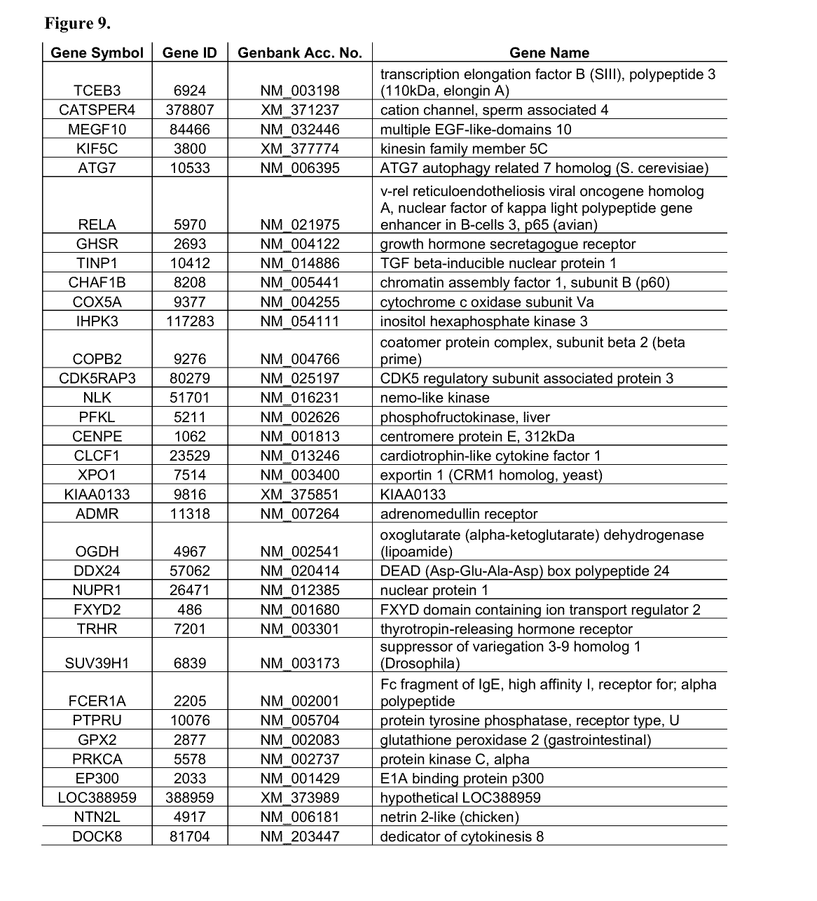

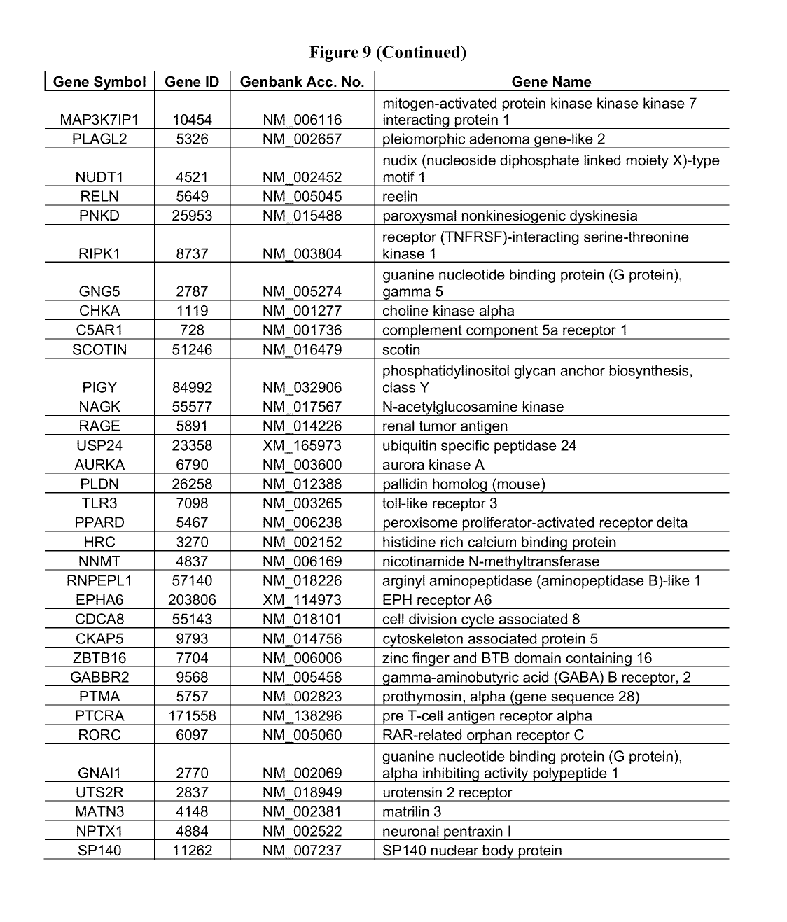

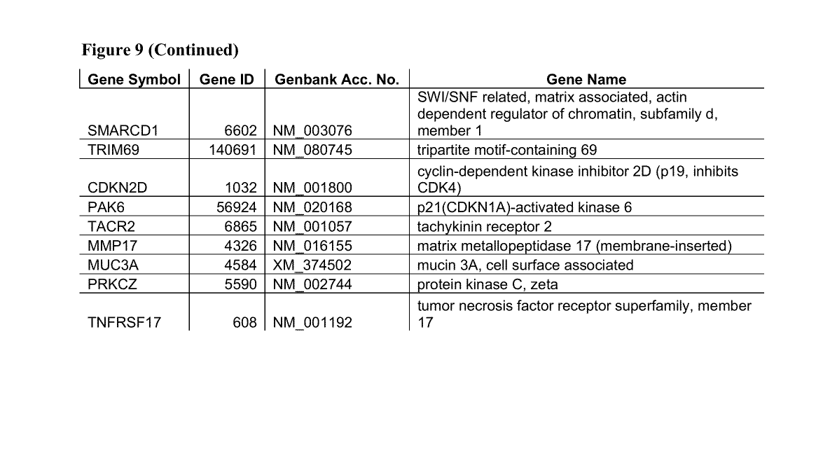

Accumulation of LC3-GFP may be due to, for example, increased initiation of autophagy or a block in degradation of autophagosomes. In order to evaluate the shape and size of the lysosomal compartment, H4 cells stably expressing lysosomal protein Lamp1-RFP were used. Knock-down of mTOR led to re-distribution as well as a significant increase in the levels of Lamp1-RFP (FIG. 8), suggesting that in addition to up-regulating autophagy, inhibition of mTOR also causes an expansion of the lysosomal compartment. Using this system it was determined that transfection of siRNAs against 78 genes (30%) led to a significant (+/−1.5 SD) change in the levels of Lamp1-RFP, which positively correlated with the changes in the levels of autophagy, suggesting that these genes regulate autophagy by altering the lysosomal function (FIG. 9).

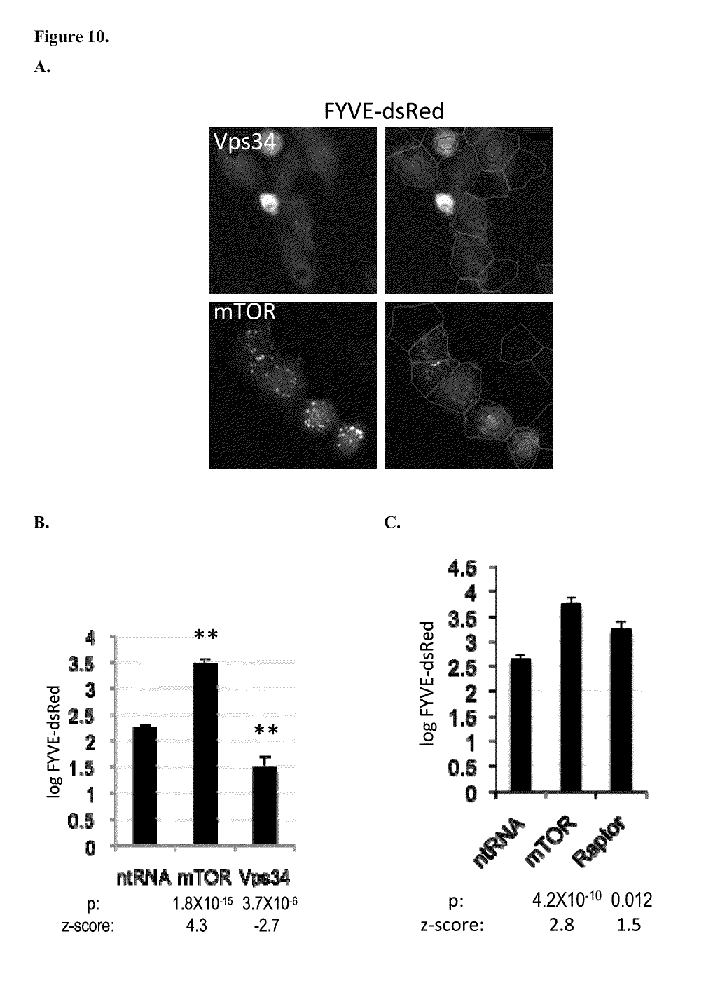

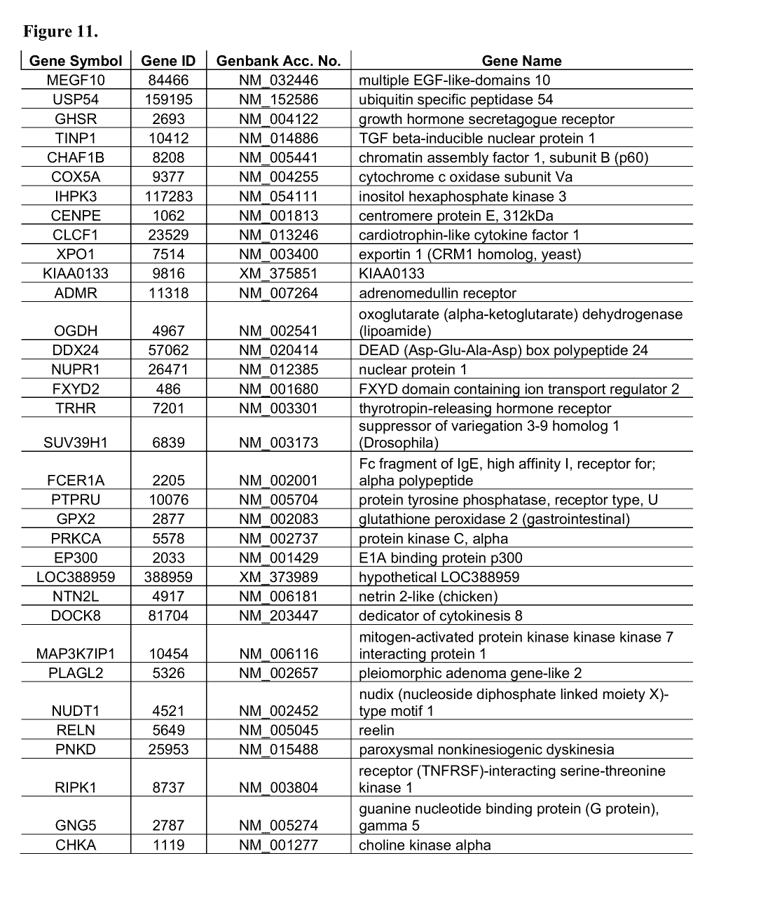

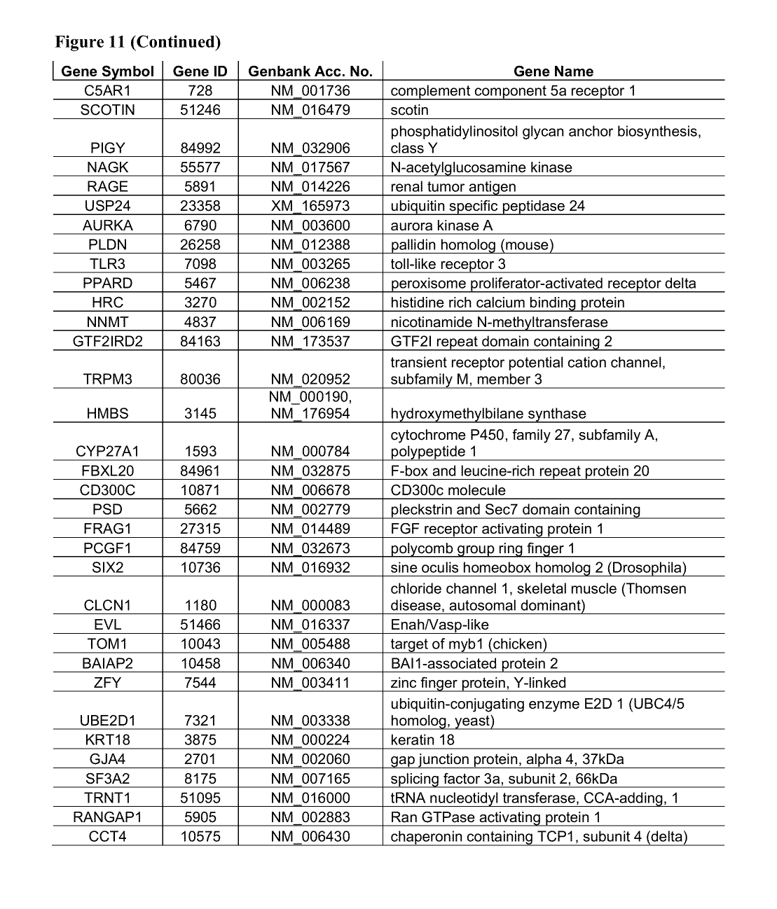

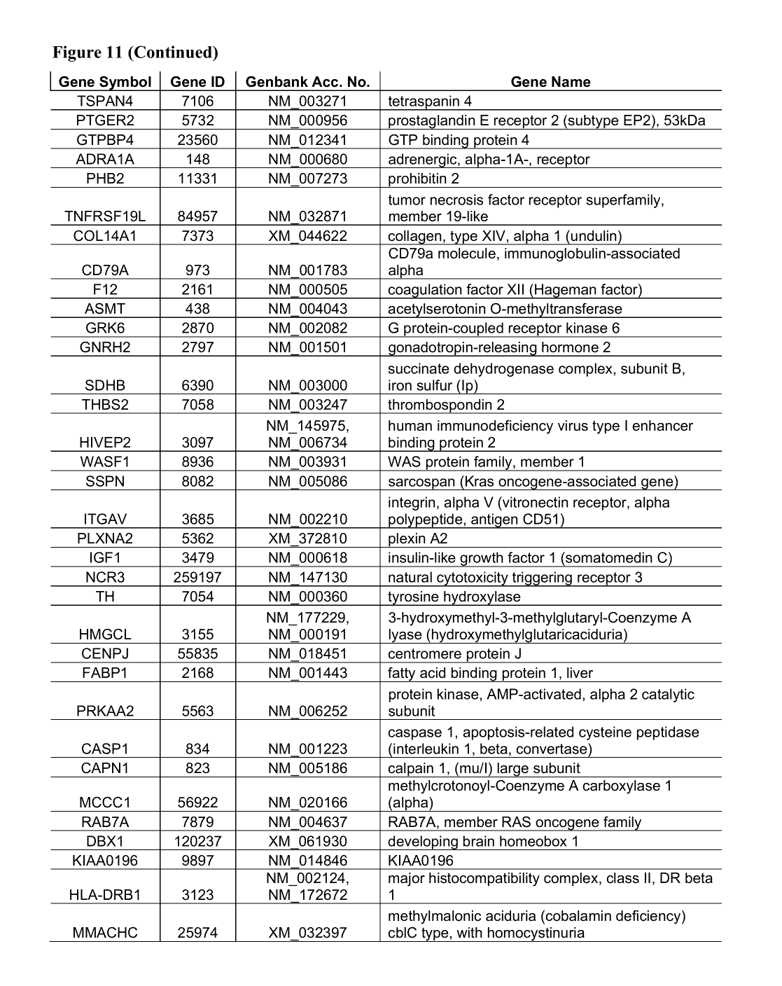

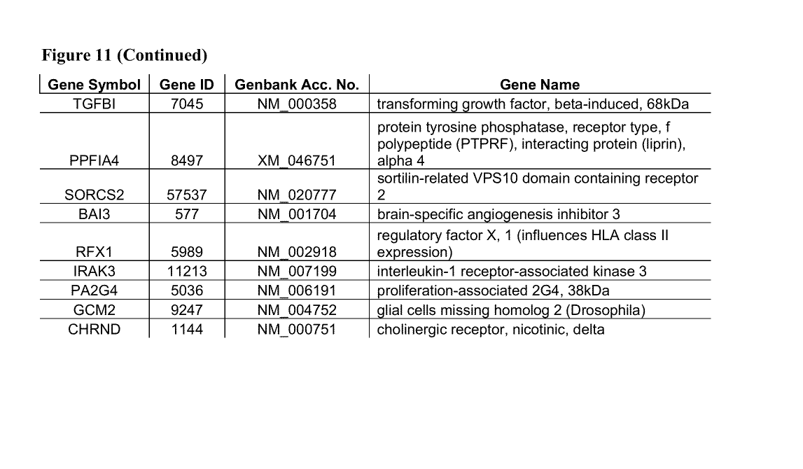

The impact of the knock-down of the individual hits on the activity of the type III PI3 kinase, an important mediator of autophagy in both yeast and mammalian cells was also determined In order to identify genes that induce or suppress autophagy by altering type III PI3 kinase activity, H4 cells stably expressing FYVE-dsRed reporter, which specifically binds to the product of the type III PI3 kinase, PtdIns3P, were used. Accumulation of PtdIns3P caused by elevated type III PI3 kinase activity results in a punctate vesicular localization of this reporter. Transfection of siRNA against Vps34, the catalytic component of the kinase, significantly decreased FYVE-dsRed vesicle recruitment (FIGS. 10A and B). Consistent with the effects of rapamycin, knock-down of mTORC1 components mTOR and Raptor strongly increased FYVE-dsRed vesicular signal (FIG. 10C). Using this system, it was also demonstrated that knock-down of 110 (47%) out of the 236 confirmed genes led to a significant (+/−1.5 SD) alteration in PtdIns3P levels, which positively correlated with the change in LC3-GFP positive autophagosome formation (FIG. 11), suggesting that these genes act upstream of the type III PI3 kinase in the regulation of autophagy. Agents that increase the levels of both LC3-GFP and FYVE-dsRed vesicle recruitment are among those likely to induce autophagic degradation.

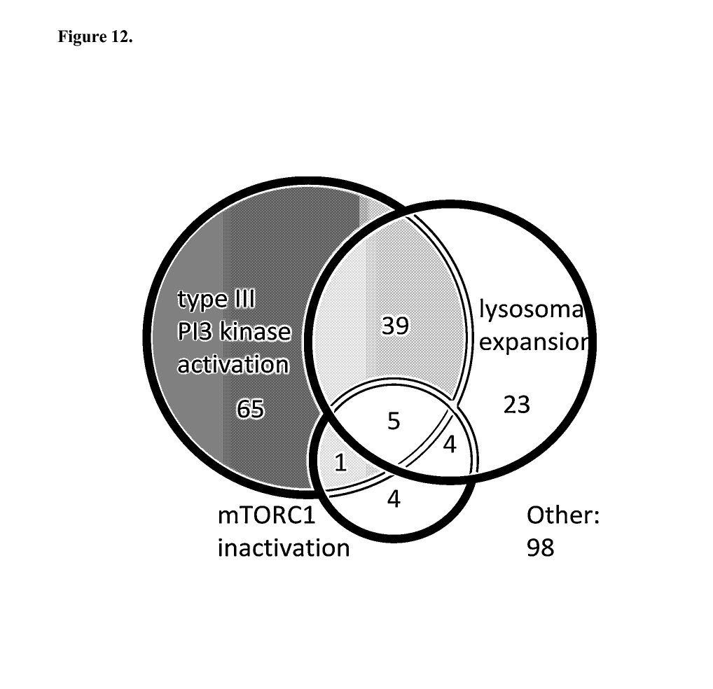

To further sub-divide the 219 genes which knock-down induced autophagy, the hits belonging to each of the subgroups identified in the secondary characterization assays were compared (FIG. 12). A substantial overlap between the hits with increased vesicular localization of FYVE-dsRed and those that accumulated Lamp1-RFP was demonstrated. Agents that inhibit the activity of this subset of genes are among those likely will simultaneously regulate the type III PI3 kinase, autophagy and lysosomal activity.

### Example 3

### Cell Death and ER Stress are not Major Contributors to the Induction of the Autophagy Induced During the siRNA Screen

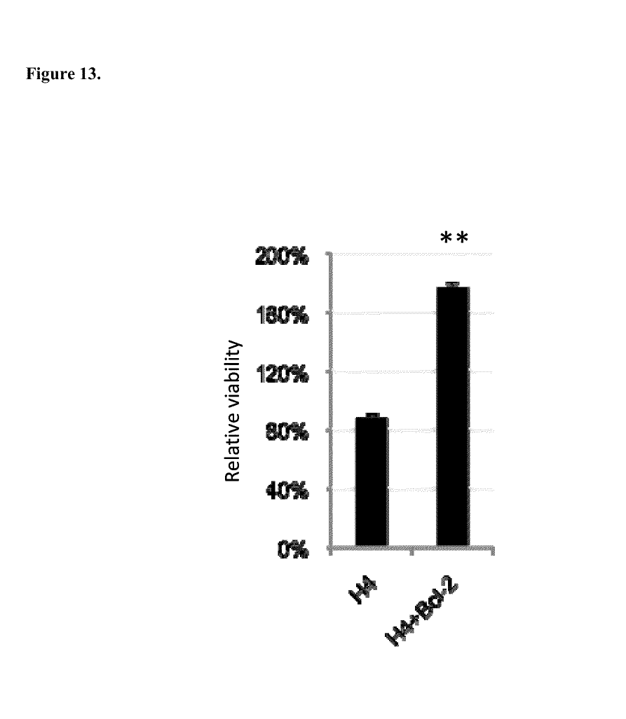

It was investigated whether the induction of autophagy observed during the siRNA screen reflected a general response to cellular stress following knock-down of an essential gene, rather than a specific function of that gene in the regulation of autophagy. Expression of Bcl-2 significantly improved average cell viability following siRNA transfection (FIGS. 13-15). With the exception of Kif 11 and integrin α5, knock-down of the 91 genes able to induce autophagy in cells expressing Bcl-2 failed to generate substantial loss of viability in these cells. This suggests that up regulation of autophagy following inhibition of these genes was not dependent on the induction of a cell death response. Of the genes which knock-down was unable to up regulate autophagy in cells expressing Bcl-2, 81 had high (over 85%) viability in wild type cells. Therefore, inhibition of the activity of 170 of the 129 identified autophagy-inhibitor genes results in the induction of autophagy through a cell-death independent mechanism.

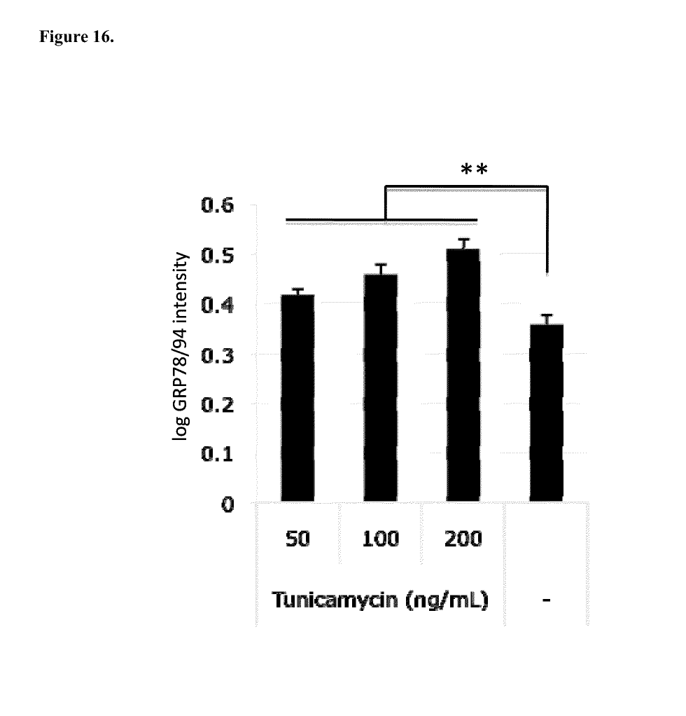

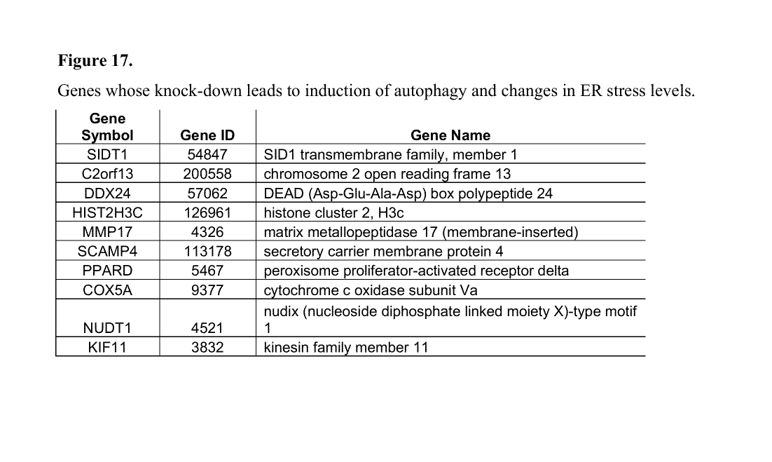

In addition to cell death, autophagy is often induced in response to various forms of cellular stress, including ER stress. In order to determine whether stimulation of autophagy in response to knock-down of our hit genes could be due to ER stress, in-cell-western assays assessing the expression levels of GRP78 and GRP94, specific markers of ER stress, were performed. Treatment with tunicamycin, a potent inducer of ER stress, led to a dose-dependent up-regulation of GRP78 and GRP94 (FIG. 16), as well as to increase in autophagy. In 97% of the genes tested (182 out of 188 genes tested, FIG. 17) there was no significant up-regulation of ER stress following knock-down of genes leading to the stimulation of autophagy. Therefore, ER stress is not a major contributor to the induction of the autophagy observed in the screen. The data therefore suggest that induction of autophagy following knock-down of the majority of the hits is due to the induction of a specific signaling event, rather than a part of a general cellular stress response induced by cell death or a result of a widespread ER stress.

### Example 4

### The Effects of Bcl-2 on Induction of Autophagy

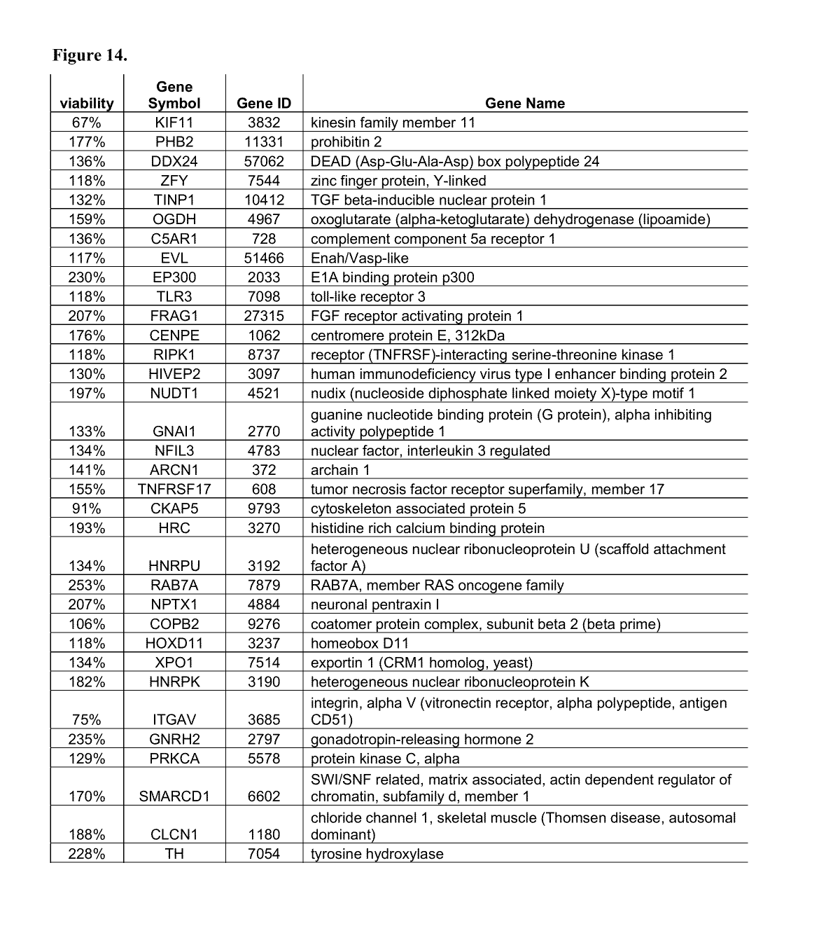

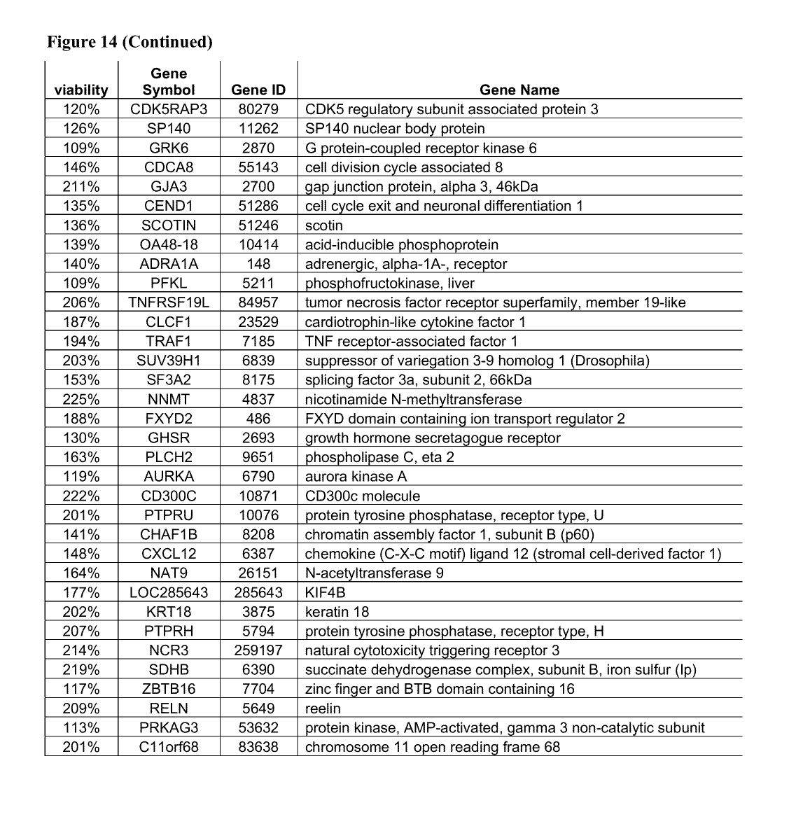

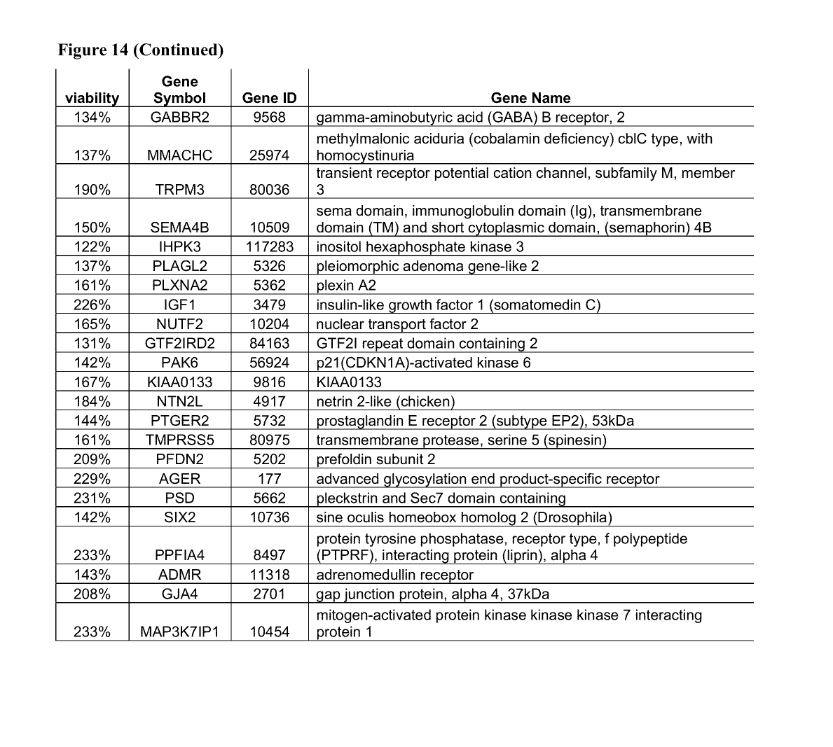

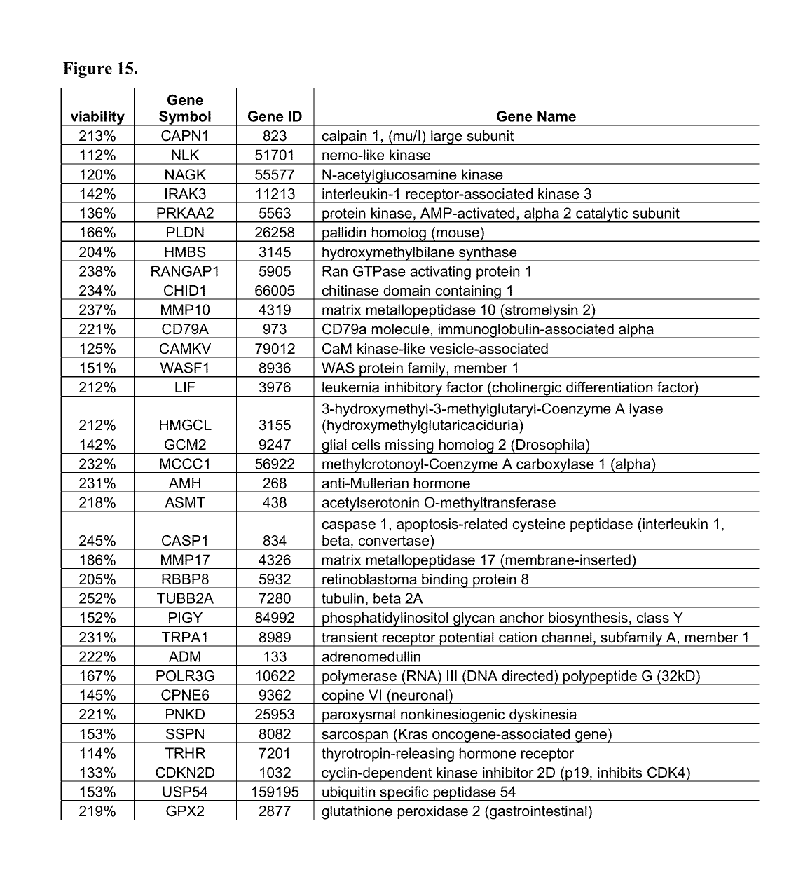

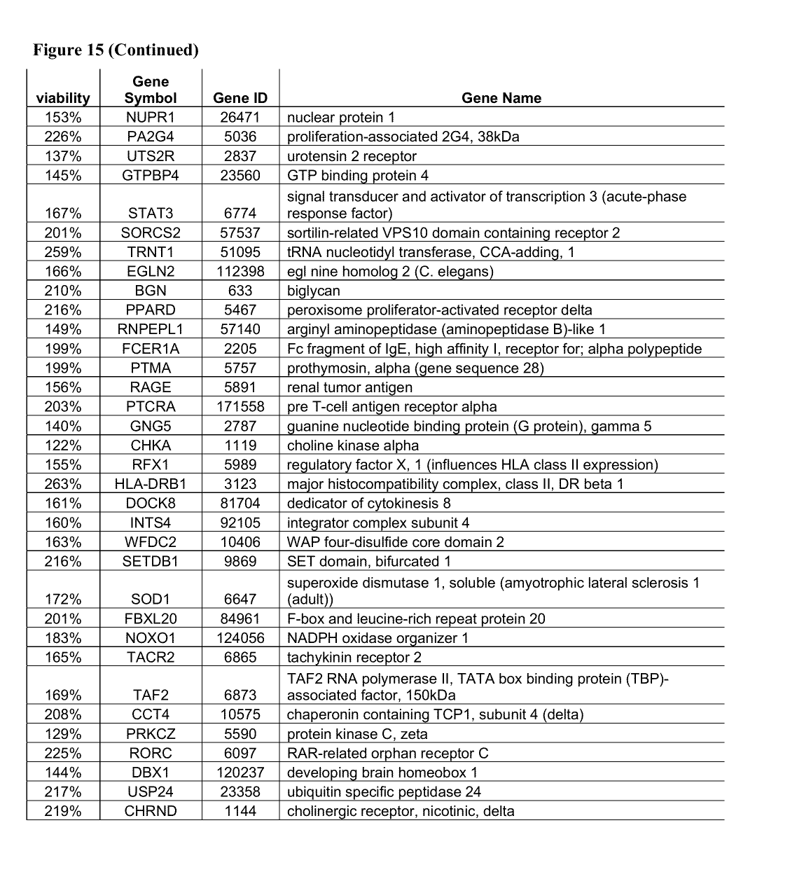

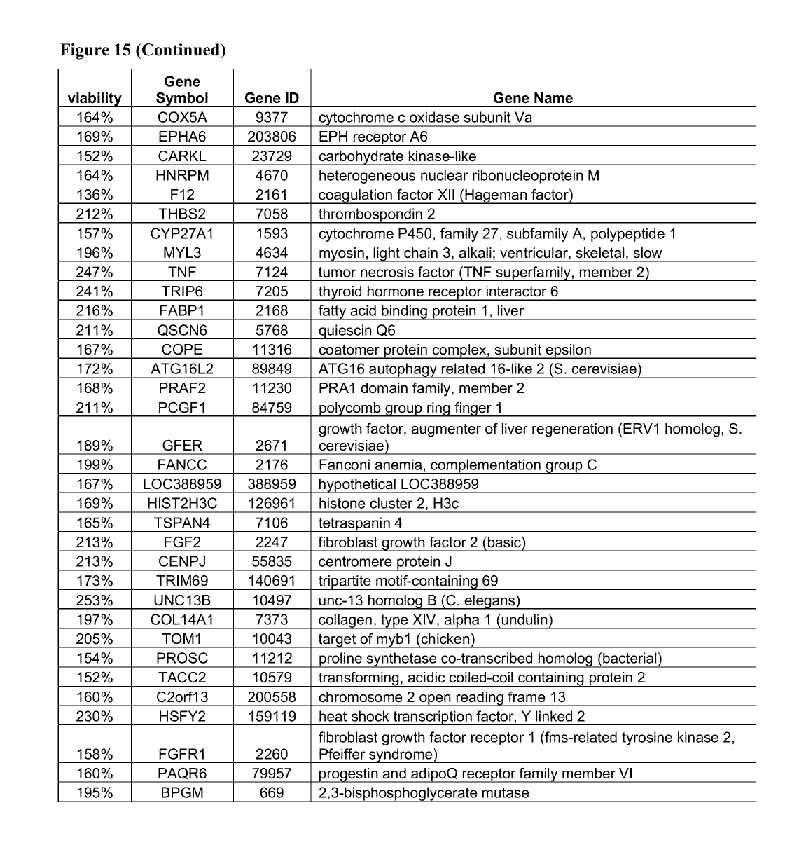

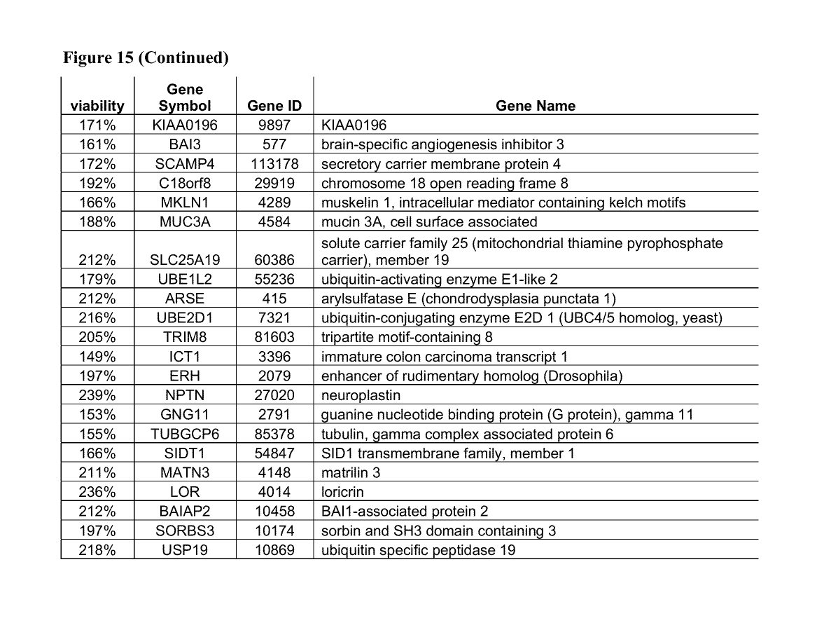

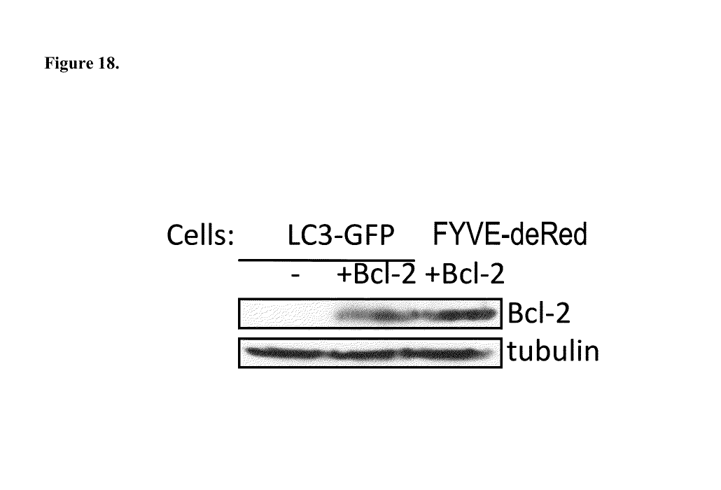

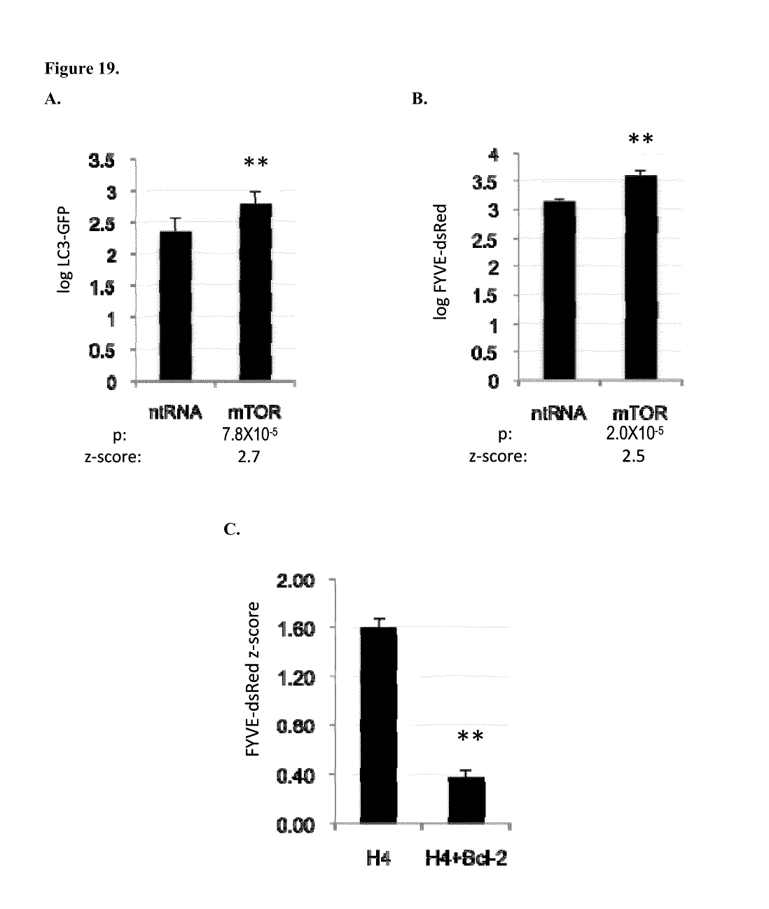

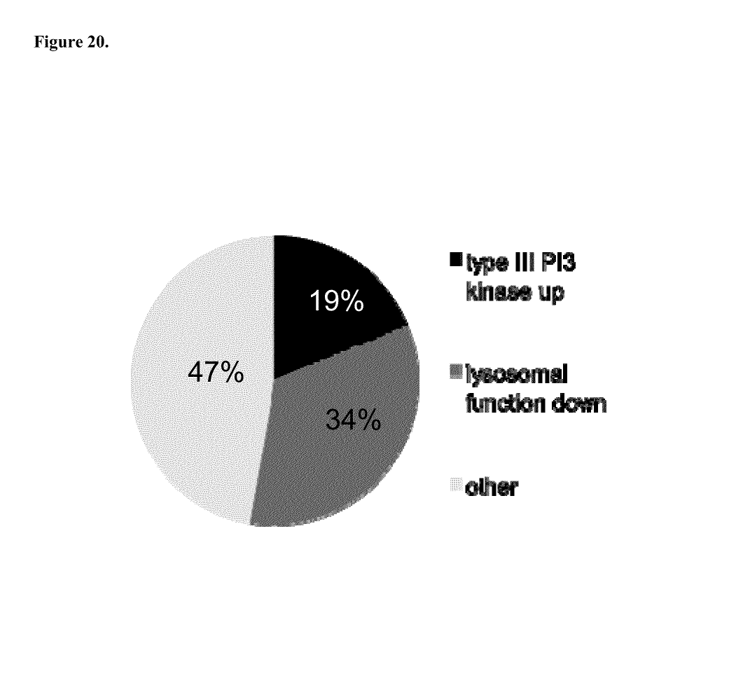

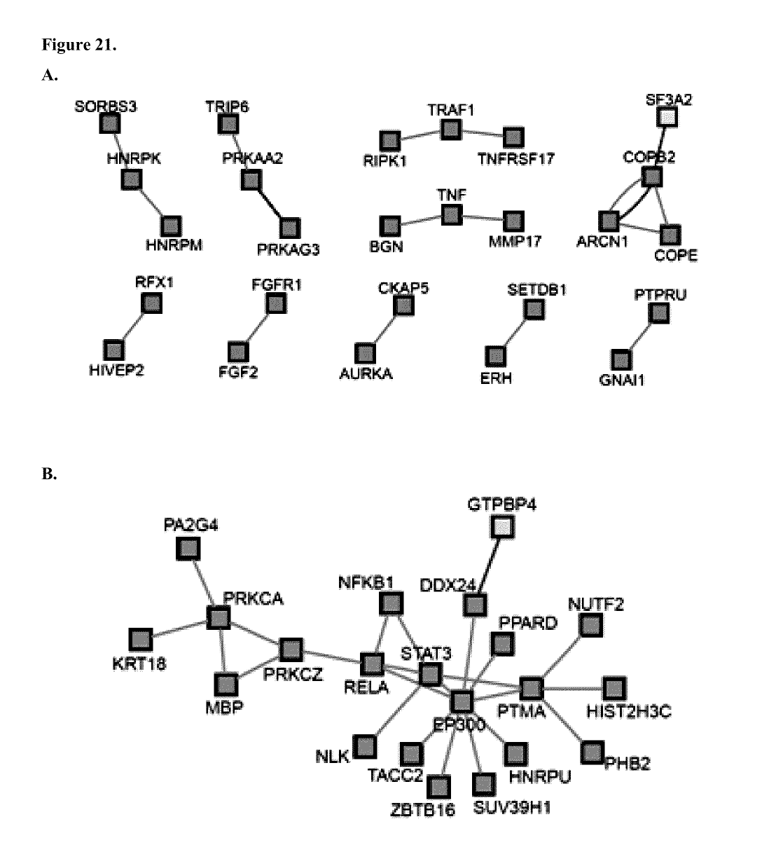

Beclin 1, the regulatory autophagy specific component of the type III PI3 kinase, was originally identified as a binding partner of the anti-apoptotic protein Bcl-2. Recently, in addition to its prominent function in regulation of apoptotic cell death, Bcl-2 has been suggested to negatively regulate autophagy through its interaction with beclin 1 and consequent inhibition of the type III PI3 kinase activity. In order to assess the function of Bcl-2, a tertiary characterization screen was performed to compare the induction of autophagy and the type III PI3 kinase activity in wild-type H4 cells and cells stably expressing Bcl-2 (FIG. 18). As a control, it was demonstrated that knock-down of mTOR was able to significantly induce both LC3-GFP and FYVE-dsRed vesicle recruitment in the Bcl-2 expressing cells (FIGS. 19A and B). Consistent with the proposed negative regulation of type III PI3 kinase by Bcl-2, a significant decrease in average FYVE-dsRed induction following knock down of the hit genes in H4 cells expressing Bcl-2 as compared to wild type controls occurred (FIG. 19C). Knock-down of 91 (42%) out of the 215 tested genes was able to induce translocation of LC3-GFP to autophagosomes in the presence of Bcl-2 (FIGS. 14 and 20). In 17 (19%) out of these 91 genes induction of autophagy was correlated with the increase in type III PI3 kinase activity as assessed by the vesicle recruitment of FYVE-dsRed, indicating that these genes are involved in additional mechanisms that regulate production of PtdIns3P downstream of Bcl-2. On the other hand, knock-down of the remaining 74 genes was able to induce autophagy without additional activation of the type III PI3 kinase. Knock-down of 31 of these genes led to Lamp1-RFP accumulation in wild type H4 cells, indicating that, in these cases, a block in lysosomal degradation may contribute to the increase in autophagy in Bcl-2 expressing cells. No changes in the lysosomal function were observed for the remaining 43 genes. Thus the inhibitory effect of Bcl-2 on type III PI3 kinase is not always incompatible with the induction of autophagy, the activation of which can be accomplished without increase in PtdIns3P levels. Finally, knock-down of the remaining 124 (58%) genes was unable to induce accumulation of vesicular LC3-GFP in cells over expressing Bcl-2 (FIG. 15).

### Example 5

### Bioinformatics Network Analysis of Autophagy-Related Genes

In order to further elucidate the biological networks involved in regulation of autophagy, interactions between the hit genes were explored by mapping their direct physical interactions based on both mammalian and yeast data. Among the hits were included multiple members of several known protein complexes (FIG. 21A), including 2 subunits of NF-κB (NFκB 1 and RelA), 3 ribonucleoproteins involved in pre-mRNA processing (HNRPK, HNRPM and HNRPNU), 3 coatamer components (CopB2, CopE and Arcn1) and 2 AMPK subunits (AMPKa2 and AMPKγ3). Additionally, a large network of interacting transcription factors and chromatin modifying enzymes centered on p300 HAT and NFκB were identified (FIG. 21B). The latter indicates that transcriptional regulation may play a critical role in the regulation of autophagy.

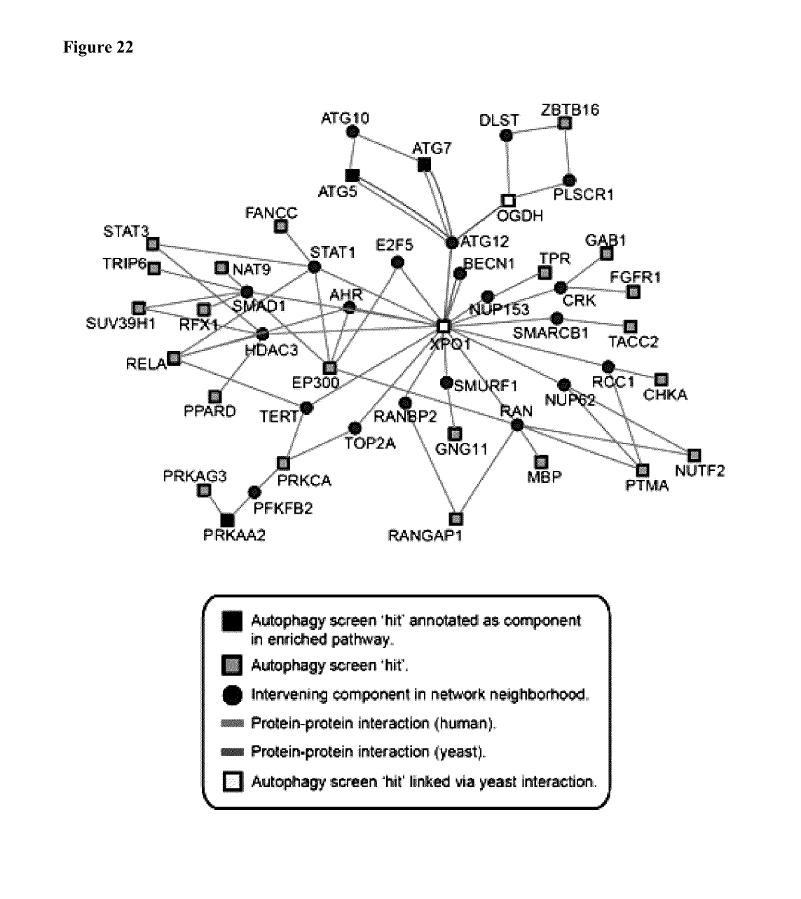

Interolog analysis (yeast-human orthologous mapping of protein-protein interactions) between the core autophagy components and the genes identified in the screen revealed that at least two of the hits, Xpo1 and OGDH, may physically interact with core autophagic machinery (FIG. 22). Xpo1 is the mammalian homolog of yeast CRM1 and an essential component of nuclear export machinery. Its interaction with Beclin1 and Atg12 likely reflects its function in the nuclear export of these proteins. On the other hand, OGDH, a metabolic enzyme localized to the mitochondrial matrix, has been reported to have cytoprotective activity independent of the enzymatic activity of the associated complex, making it a candidate for the regulation of autophagy induced by mitochondrial damage.

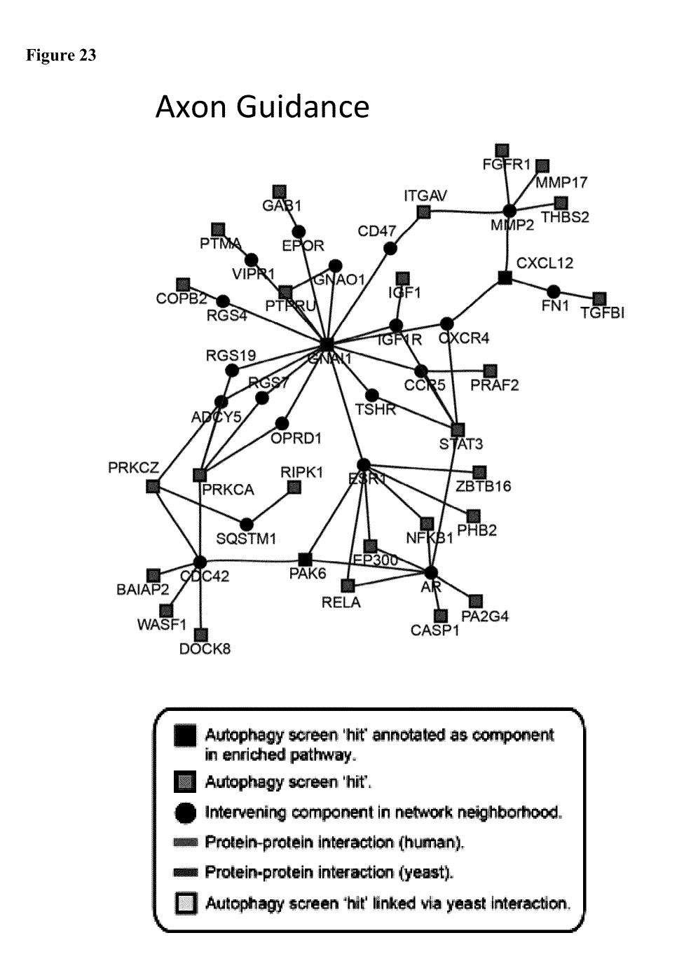

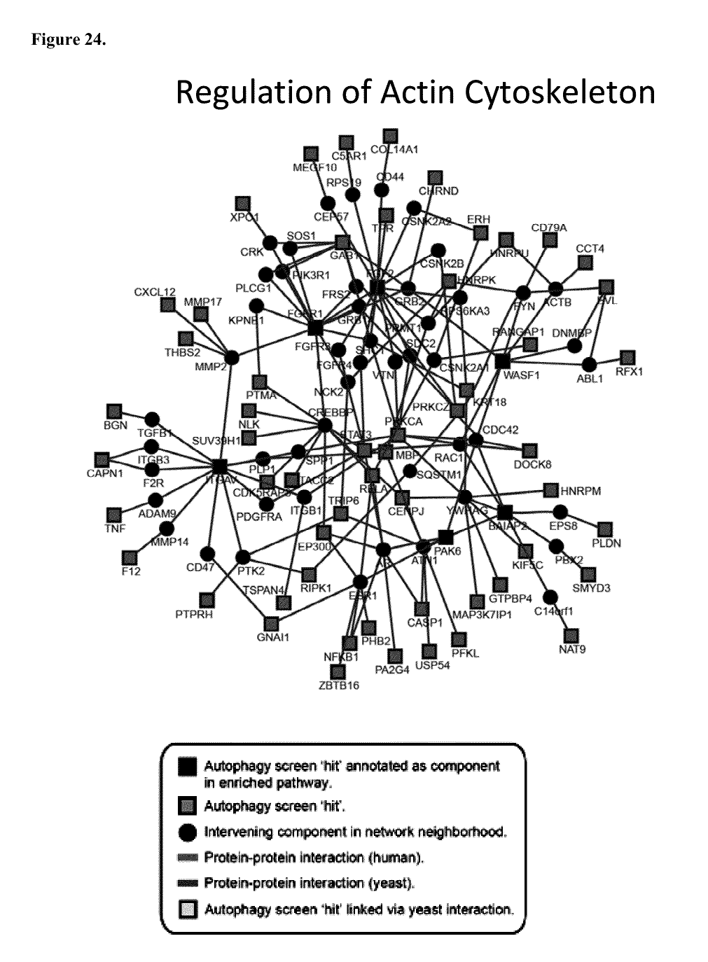

In order to investigate the connection between autophagy, axon guidance and actin dynamics, a protein-protein interaction network anchored by the hit genes belonging to these canonical pathways was generated (FIGS. 23 and 24). This analysis revealed two related networks encompassing, respectively, 27 and 61 of the hit genes.

These analyses indicate that autophagy can be modulated through the use of agents that modulate the activity of specific pathways and complexes identified herein as being associated with the regulation of autophagy.

### Example 6

### The Use of Cytokines in the Modulation of Autophagy

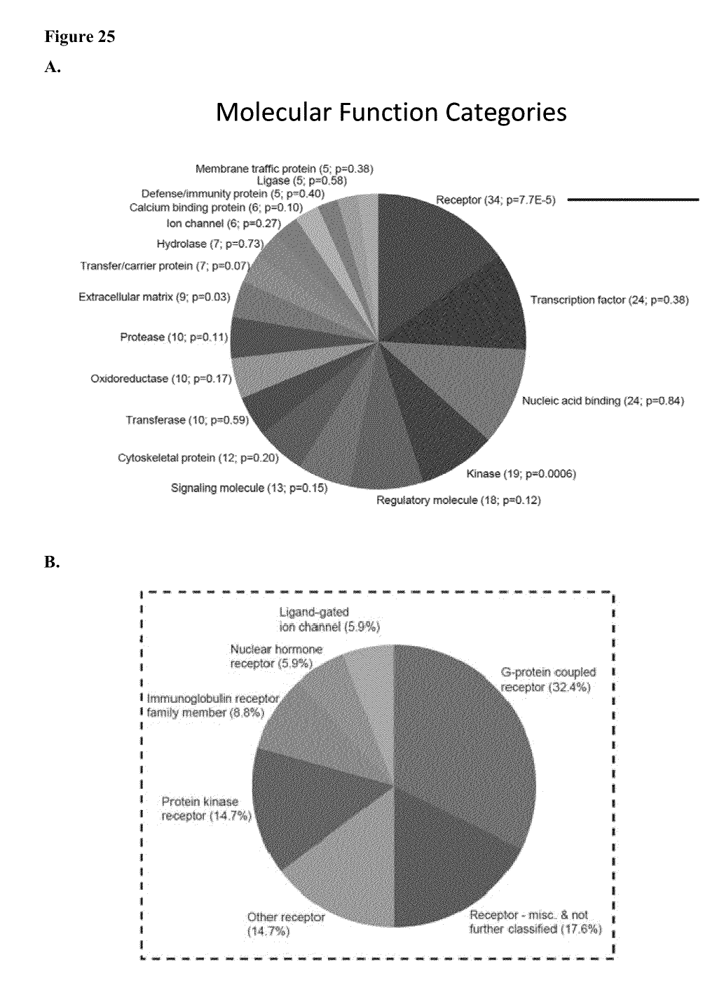

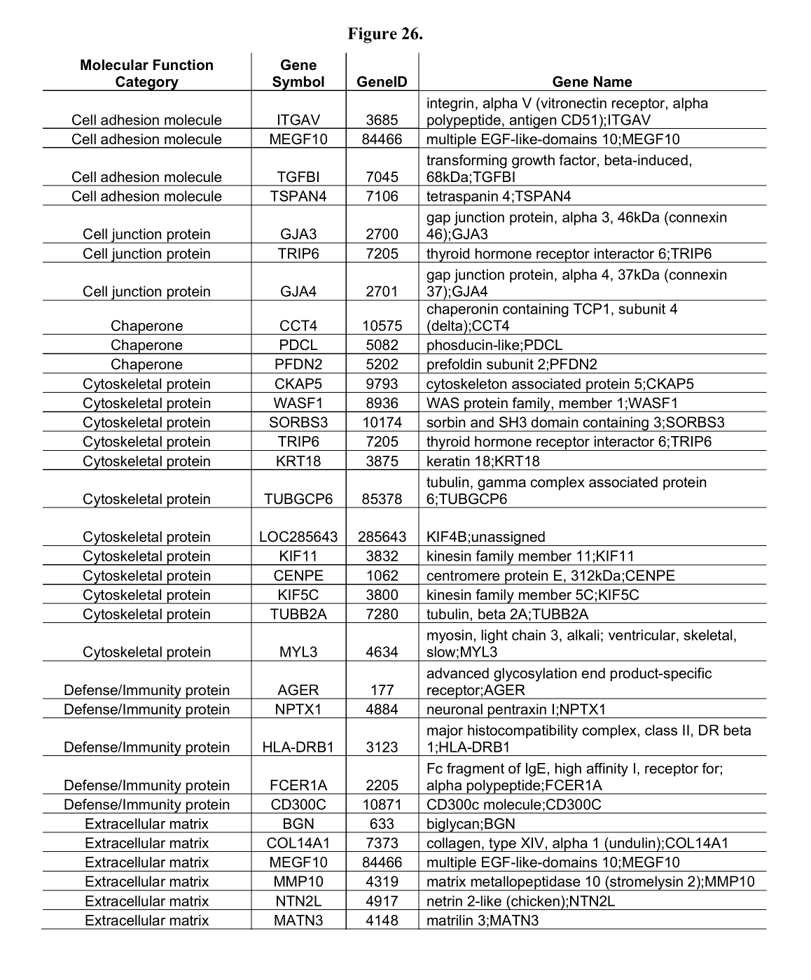

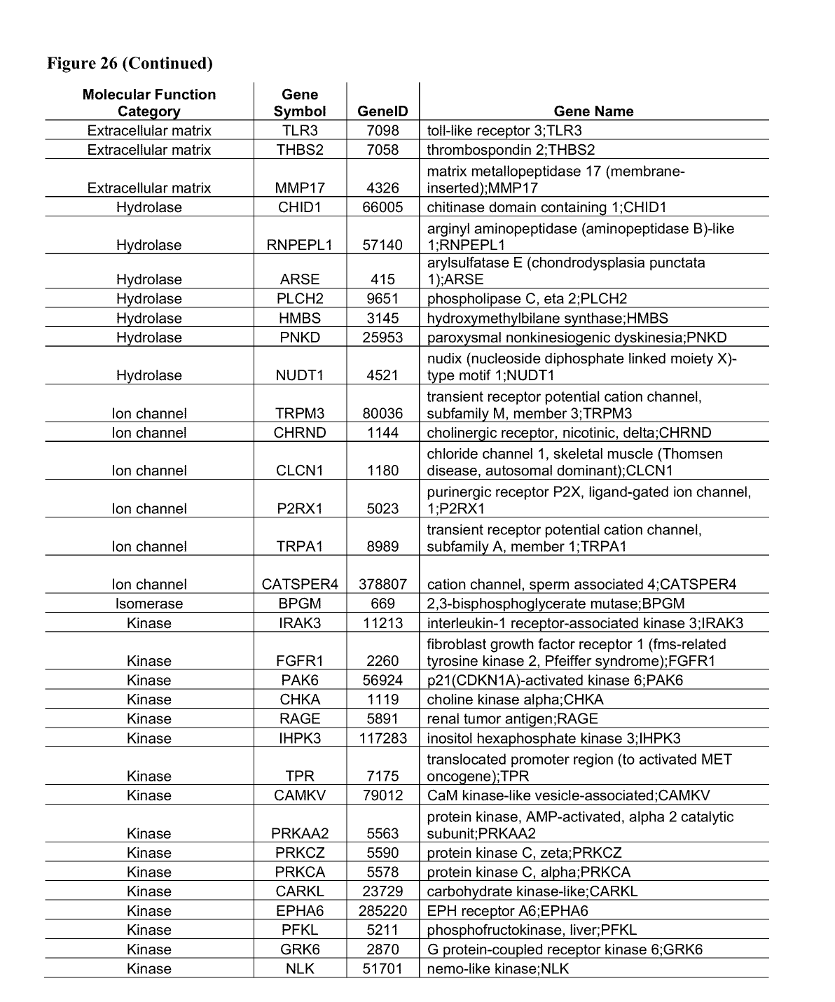

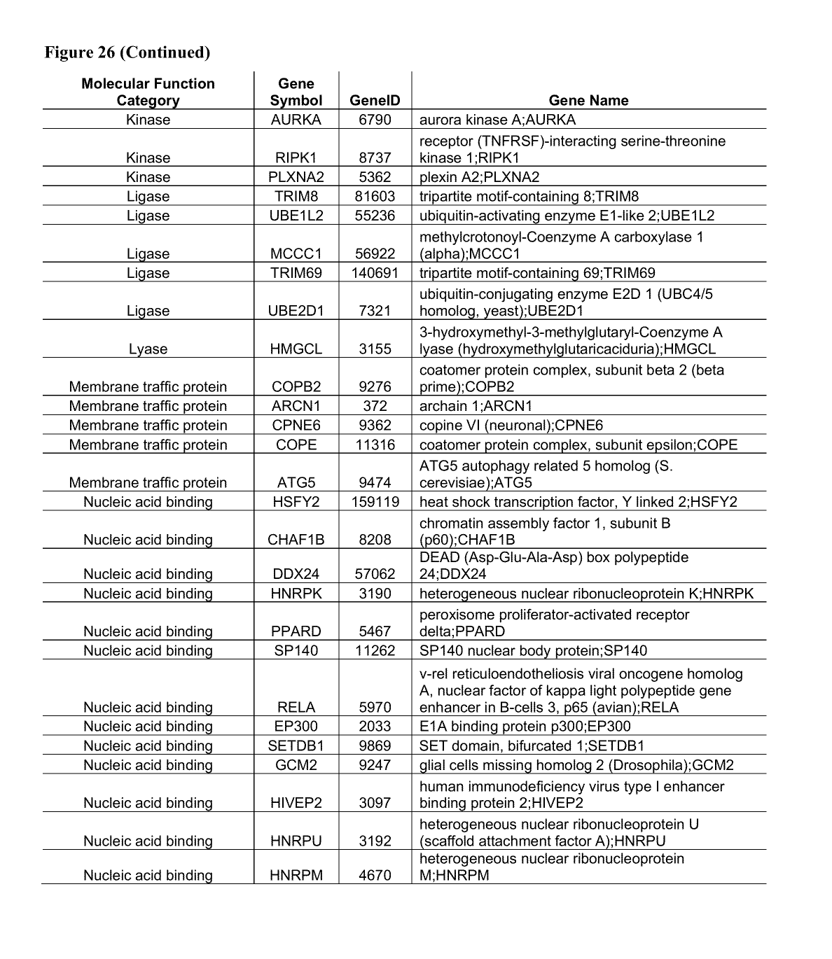

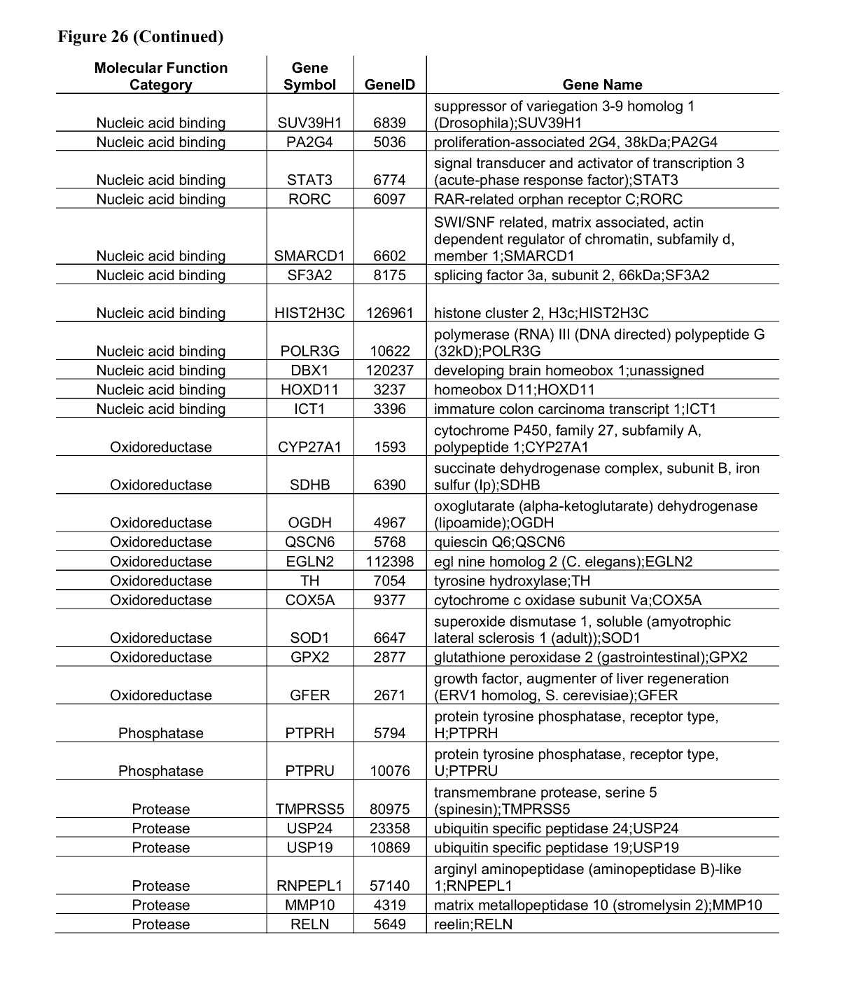

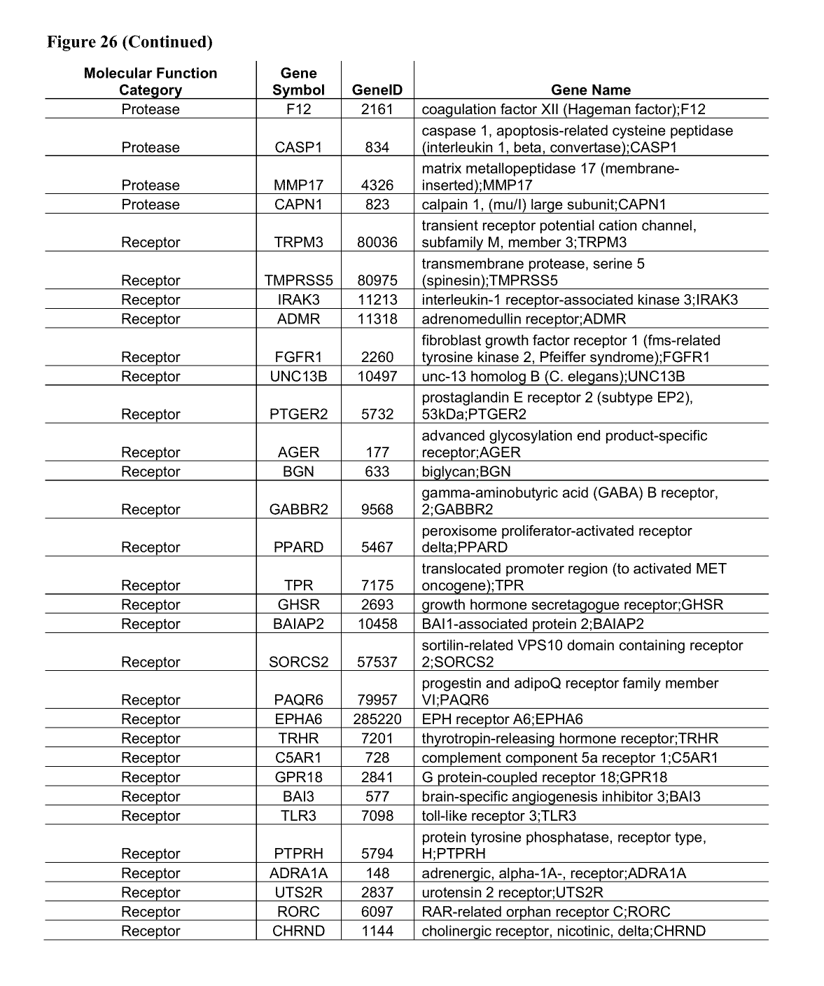

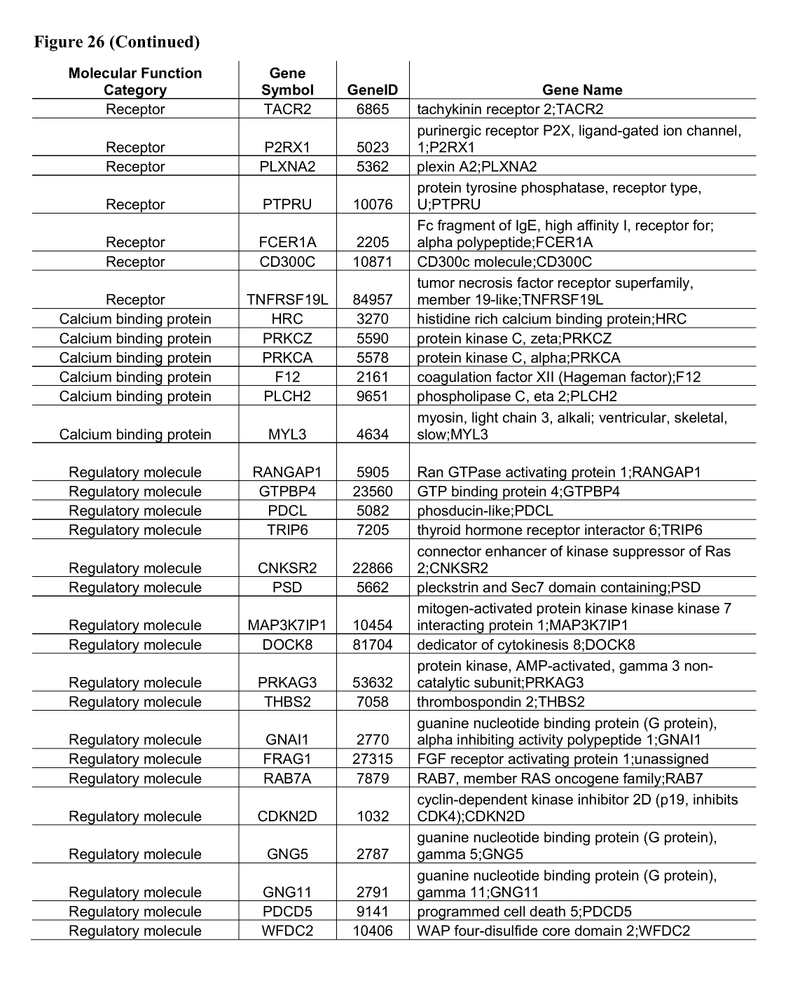

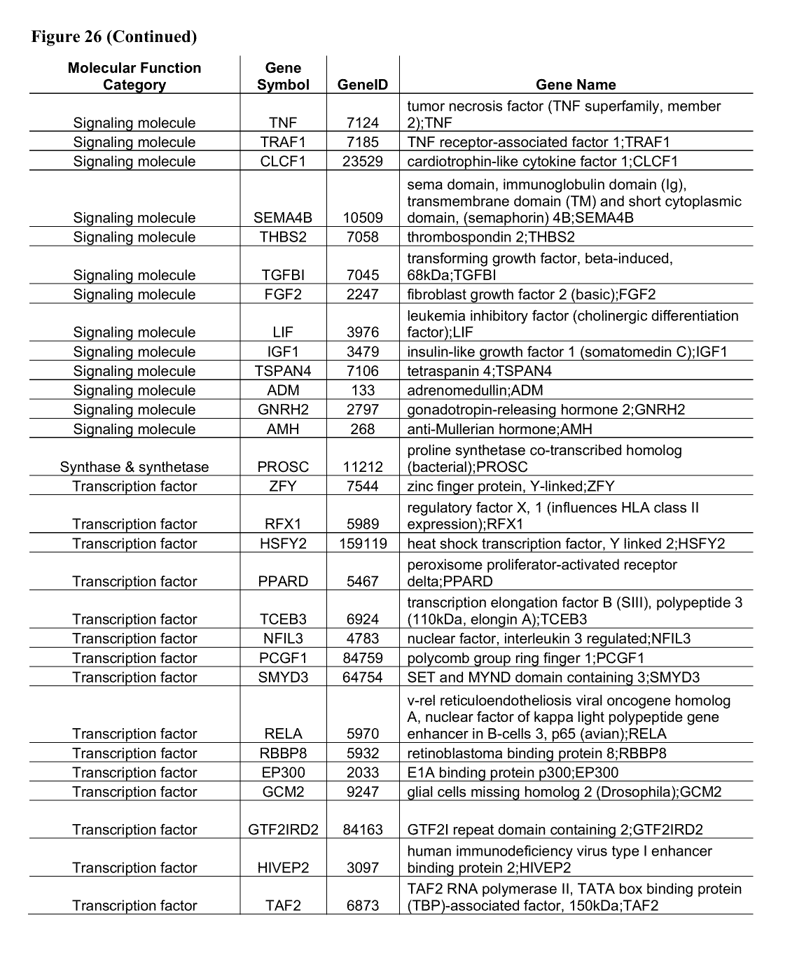

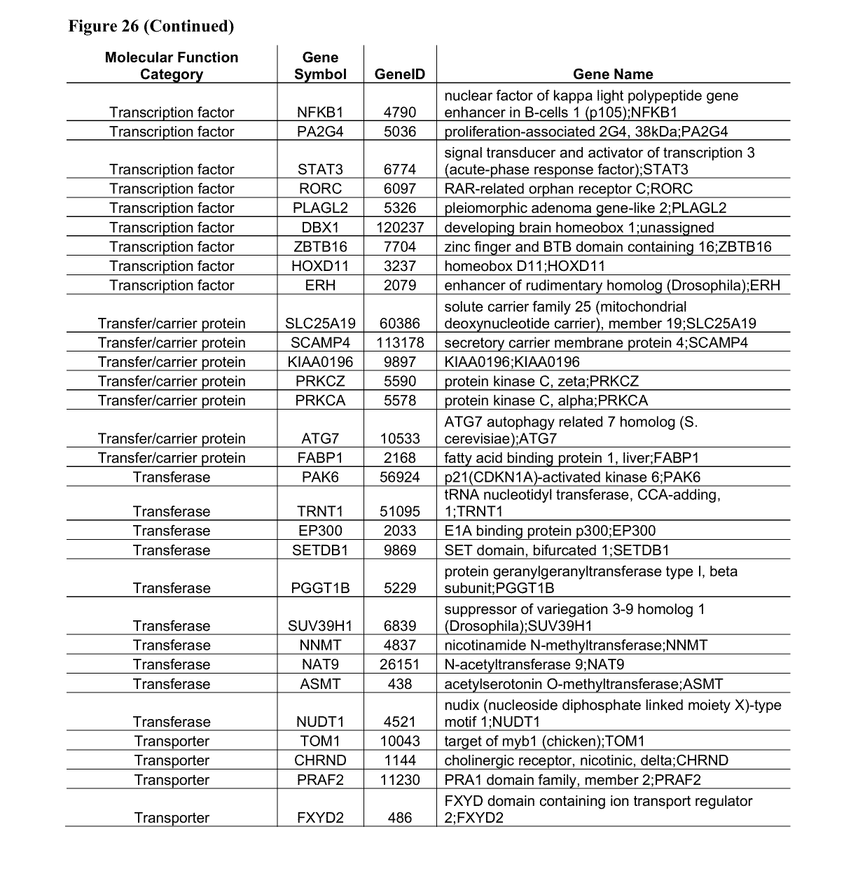

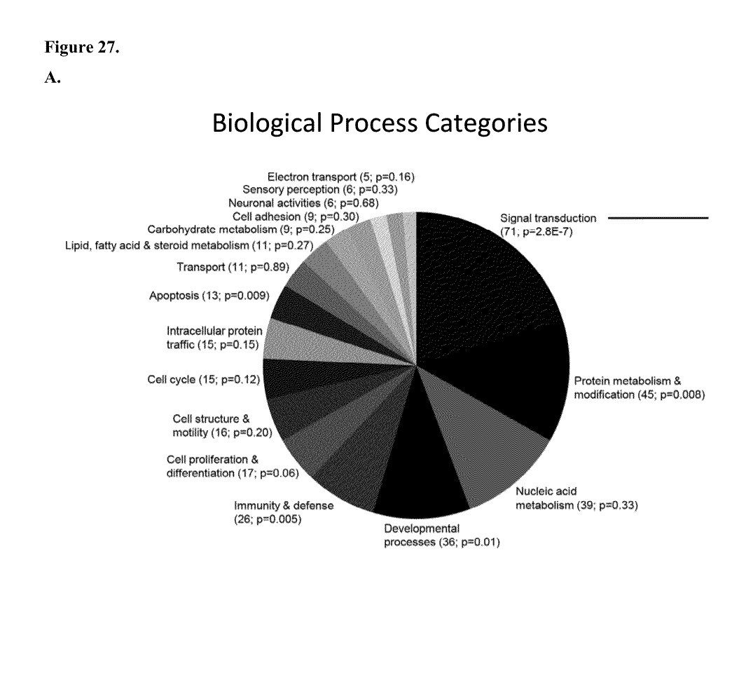

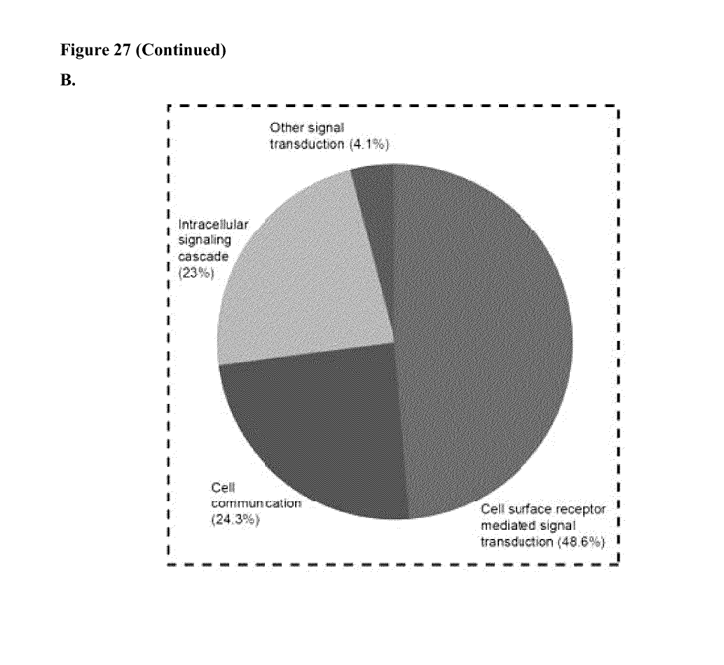

Molecular function analysis of the 236 confirmed hits using Gene Ontology (GO) revealed a highly significant enrichment in genes encoding kinases (p=0.0006), proteins with receptor activity (p=7.7×10⁻⁵) and extracellular matrix proteins (p=0.03) (FIGS. 25 and 26). The latter categories indicate that the extracellular environment, including the presence of growth factors, hormones and cytokines, plays a role in the regulation of autophagy under normal nutritional conditions. The results of GO biological process analysis also demonstrated significant enrichment in signaling molecules (p=2.8×10⁻⁷) (FIG. 27A). In agreement with the proposed function of extracellular factors in regulation of autophagy, further subdivision of these signaling molecules revealed that the largest sub-group (49%) was involved in cell surface receptor signal transduction (FIG. 27B).

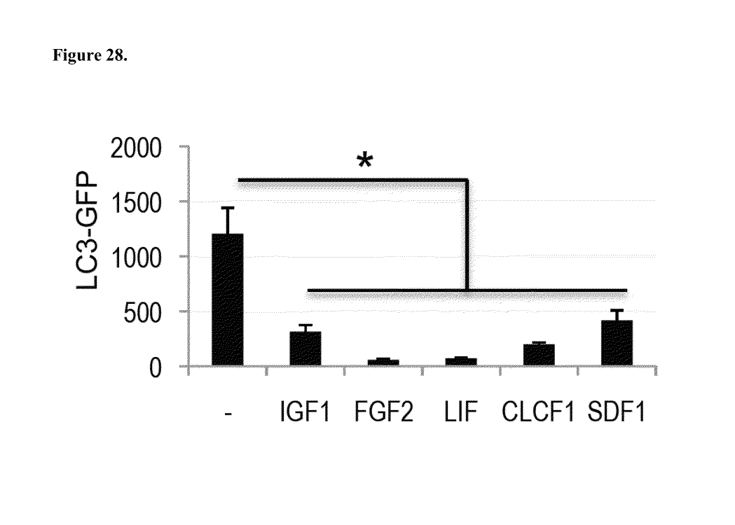

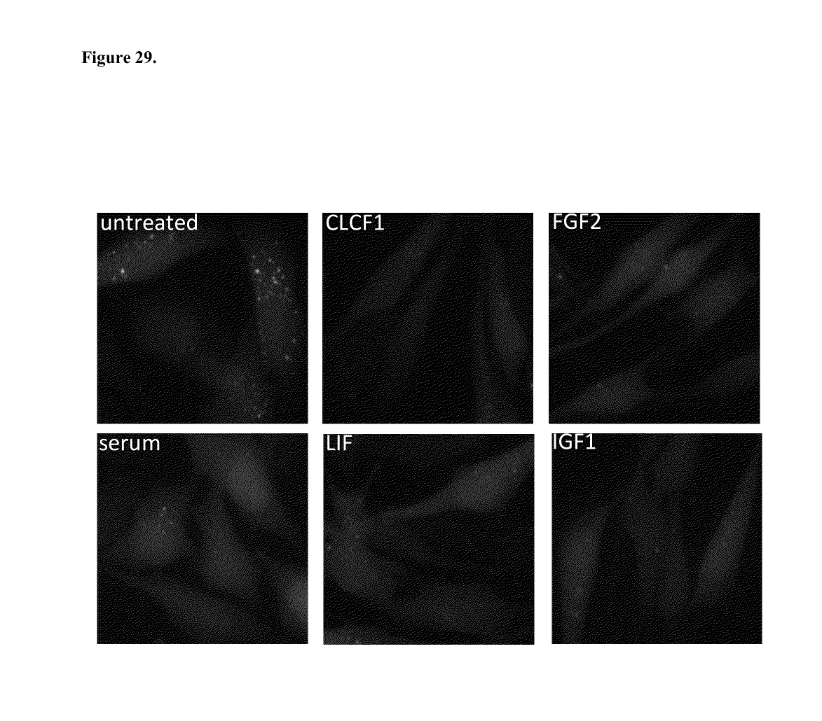

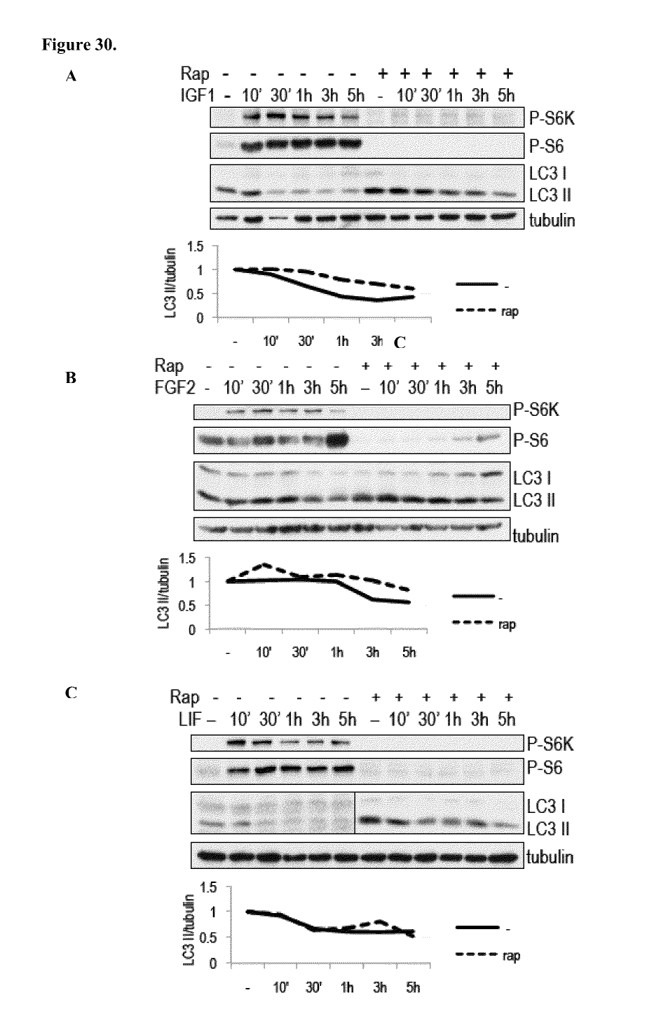

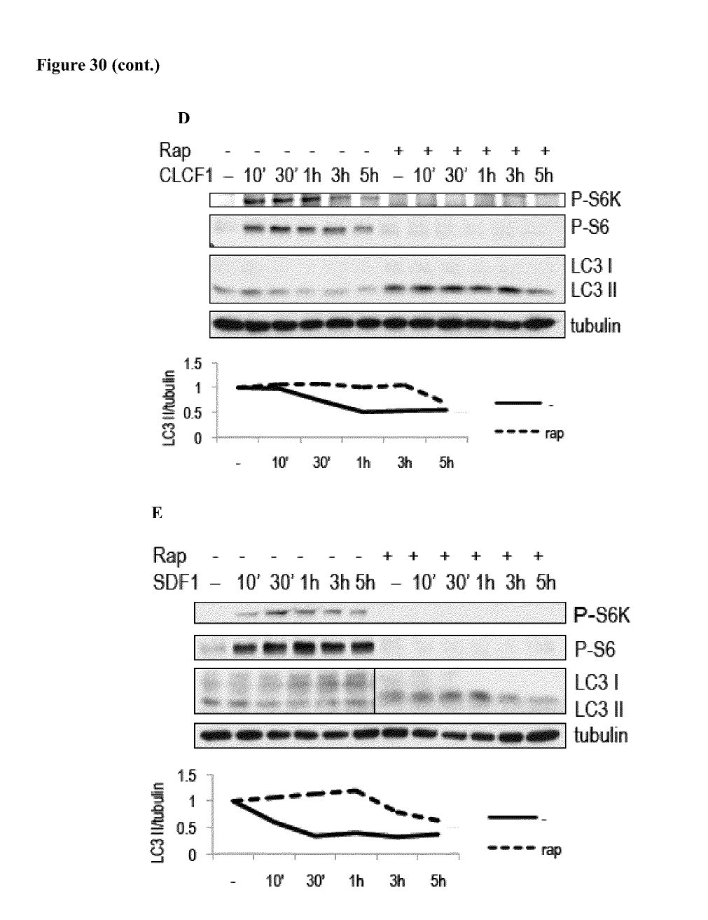

Cells were treated with several of the cytokines and growth factors identified as hits in our screen. Based on the results of the characterization assays, knock-down of IGF1, FGF2, LIF, CLCF1 and the chemokine SDF1 (CXCL12) resulted in mTORC1 independent increase in initiation of autophagy. In agreement, treatment of H4 LC3-GFP cells grown in a serum-free medium with any of these cytokines led to a significant down-regulation of autophagy as measured by LC3-GFP translocation (FIGS. 28 and 29). This data was confirmed in multiple cell lines (H4, HEK293, HeLa and MCF7) by western blot (FIG. 30). In agreement with the proposed function of cytokines in the regulation of autophagy, cells cultured in their absence displayed high basal levels of autophagy as assessed by accumulation of LC3II, which was partially suppressed by the addition of even single cytokines identified in the screen. Thus, the identified cytokines and growth factors are both necessary and sufficient for the regulation of autophagy.

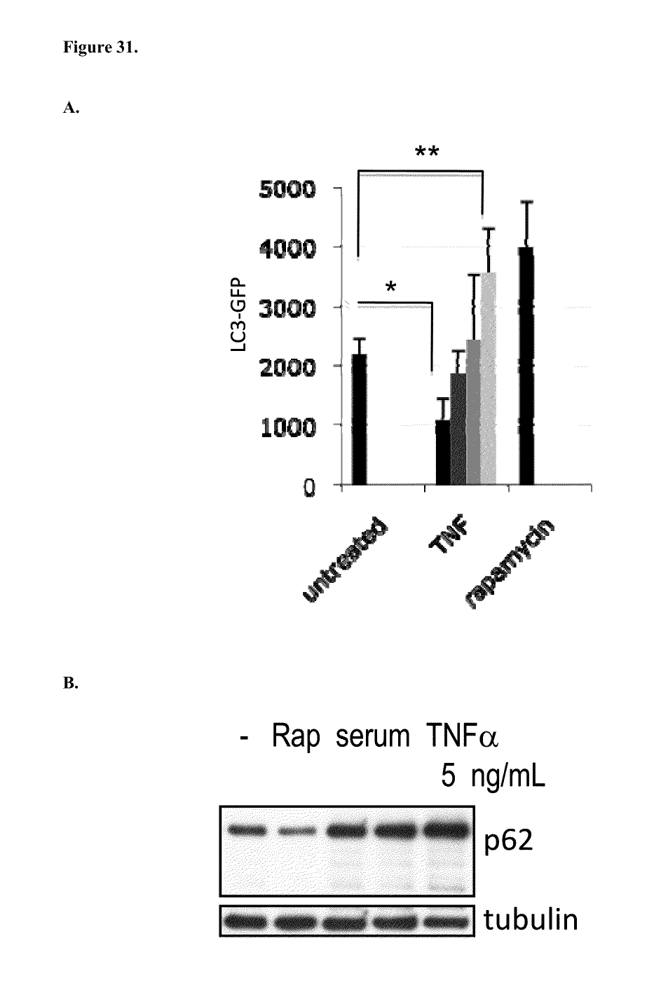

In the screen described above, knock-down of the TNF gene led to an increase in the formation of LC3-GFP positive autophagosomes, indicating a negative role for this cytokine in the regulation of basal autophagy. In order to further investigate the role of TNFα in autophagy, H4 LC3-GFP cells grown in a defined medium were treated with increasing doses of TNFα. Low doses of TNFα led to down-regulation of autophagy, while higher doses led to up-regulation of autophagy (FIG. 31A). This was confirmed by western blot showing a significant accumulation of p62 following treatment with low levels of TNFα (FIG. 31B). Since physiological levels of TNFα are very low, this suggests that this cytokine normally functions as a negative regulator of autophagy. On the other hand, increased concentrations of TNFα under pathological conditions lead to up-regulation of autophagy.

### Example 7

### The Function of NF-κB in the Regulation of Autophagy

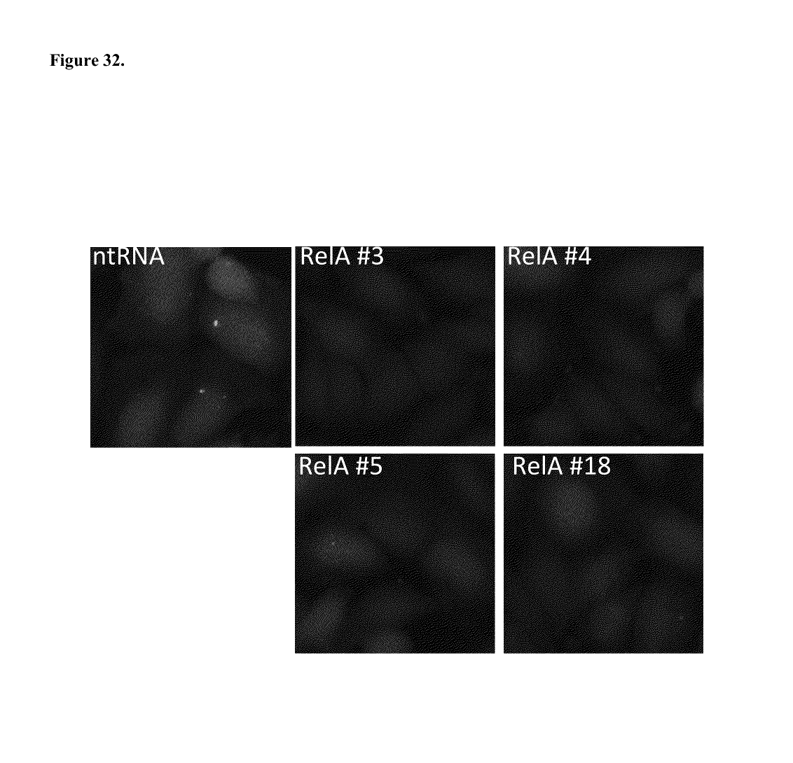

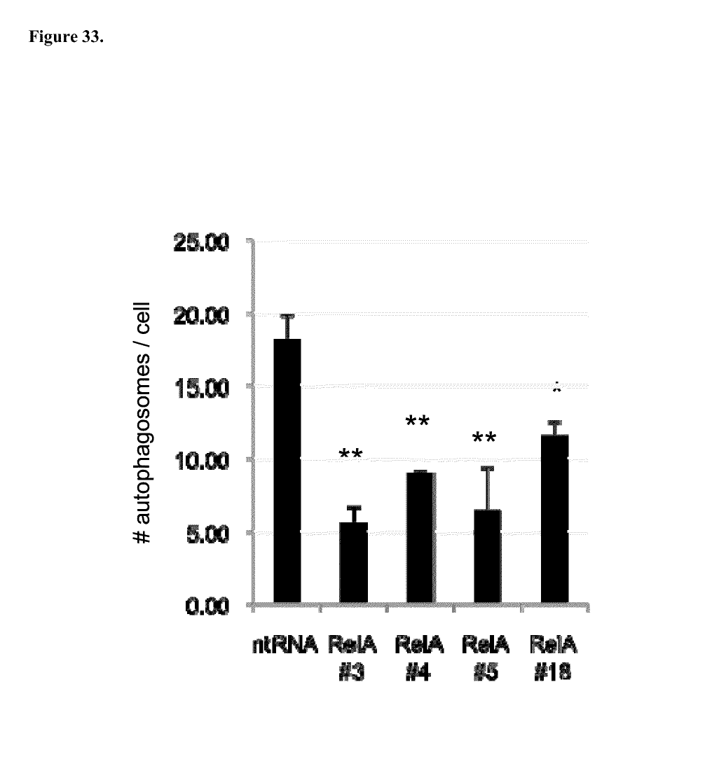

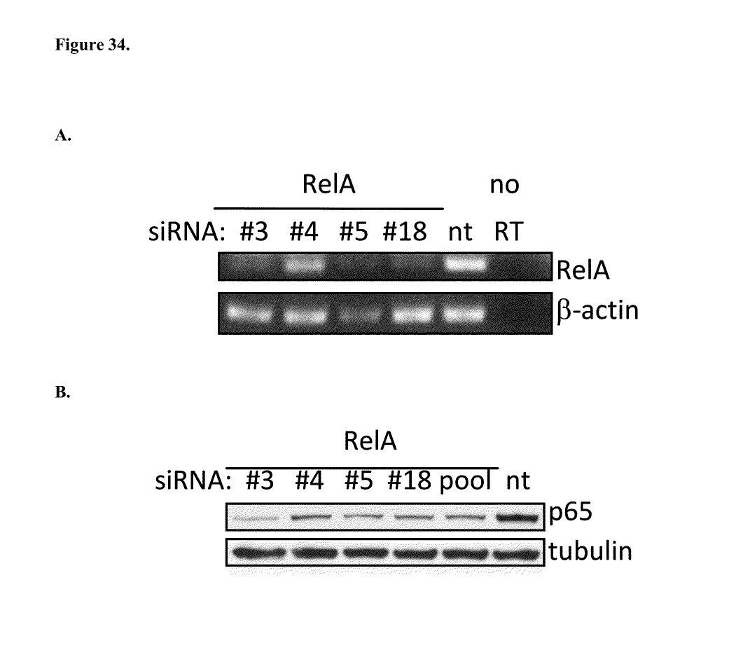

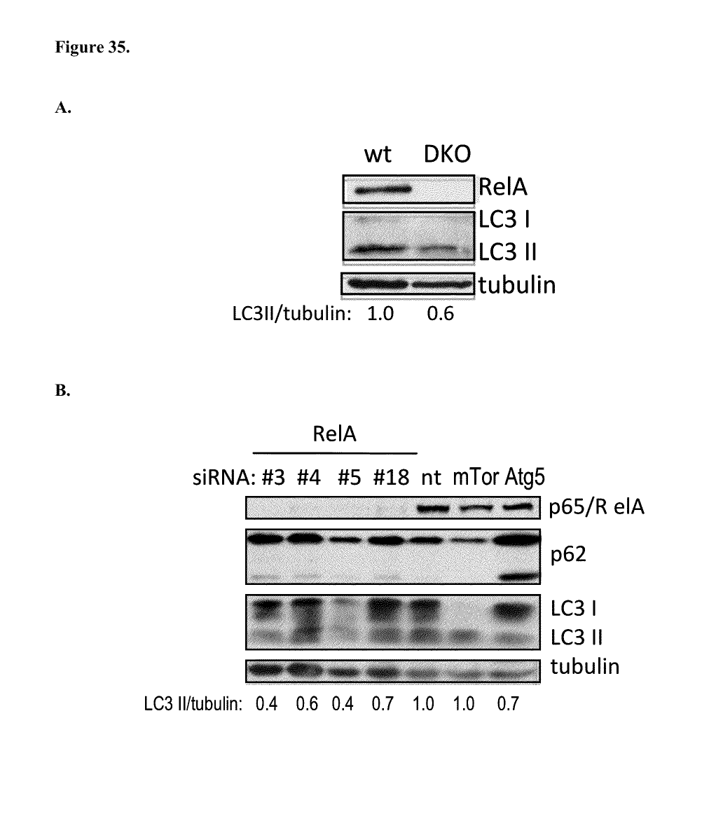

The canonical pathway analysis described above demonstrated enrichment of autophagy hits in the NF-κB (p=8.7×10⁻⁶) and RelA (p=1.2×10⁻⁶) pathways. As a validation of the screen, H4 LC3-GFP cells transfected with siRNAs against RelA were individually imaged. The levels of autophagy by quantifying translocation of LC3-GFP by fluorescence microscopy were assessed using an alternative low-throughput method. In agreement with our screen results treatment with all 4 oligonucleotides against RelA lead to strong down-regulation of number and intensity of autophagosomes (FIGS. 32 and 33). Confirming that the observed differences in the levels of autophagy were due to the knock-down of the target genes, a strong down-regulation of RelA at both mRNA (FIG. 34A) and protein level (FIG. 34B) was observed. In order to confirm that the findings regarding the function of NF-κB as a positive mediator of autophagy are not restricted to H4 cells, levels of autophagy in wild-type and double knock-out RelA^{-/-}; NF-κB^{-/-} (DKO) MEFs and in human breast cancer MCF7 cells transfected with either siRNA were compared against RelA or control non-targeting siRNA. Absence or down-regulation of RelA/NFκB expression led to suppression of autophagy as assessed by decrease in LC3 II and accumulation of p62 (FIG. 35). These data confirm NFκB as a positive regulator of basal autophagy.

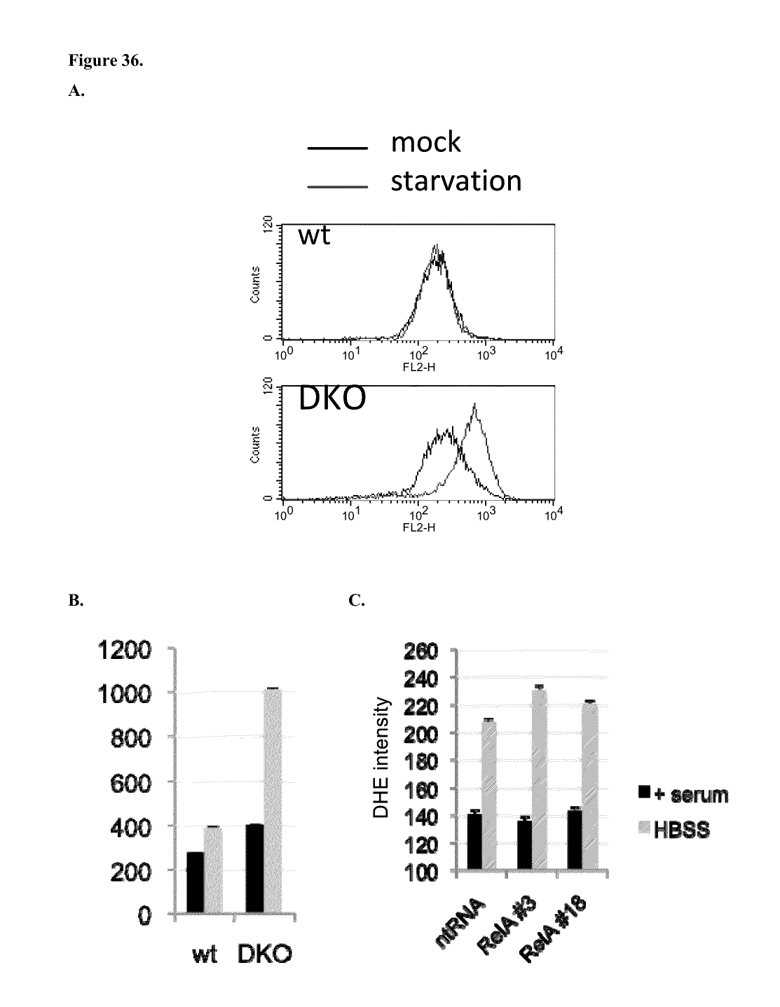

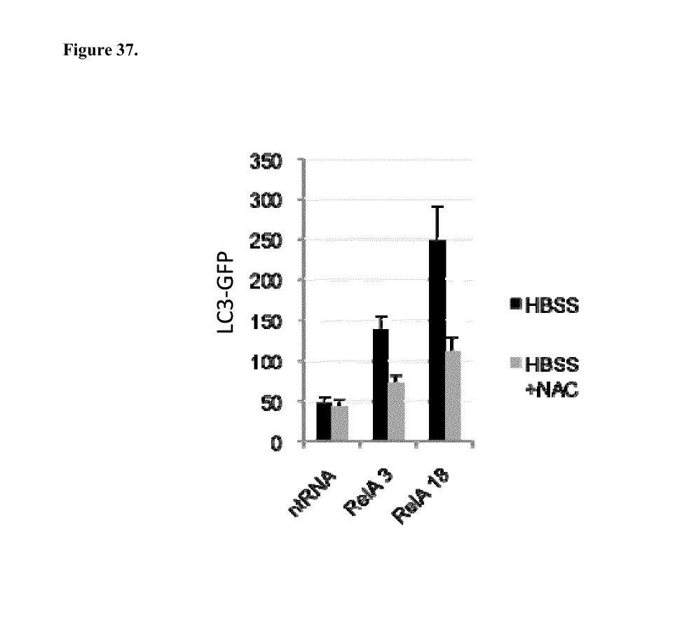

In contrast with the results described herein, NF-κB activation has been previously reported to negatively regulate autophagy associated with cell death induced in response to noxious stimuli such as nutrient starvation or death receptor ligation (Djavaheri-Mergy et al., J. Biol. Chem. 281, 30373-30382 (2006)). Since reactive oxygen species (ROS) have been proposed to participate in the mediation of starvation-induced autophagy, it was hypothesized that, under conditions of nutrient deprivation, down regulation of autophagy may be the result of the attenuation of ROS production by NF-κB. Wild type and dKO MEFs and H4 LC3-GFP cells transfected with either non-targeting siRNA or siRNA against RelA were subjected to nutrient starvation. Starvation of RelA/NF-κB deficient cells led to higher ROS accumulation than observed in wild type controls (FIG. 36). The elevated induction of autophagy observed in response to starvation in RelA deficient H4 cells was attenuated in the presence of the antioxidant N-acetyl-L-cysteine (NAC) (FIG. 37).

These data indicate that, while NF-κB plays a positive function in regulation of basal autophagy, its ability to attenuate ROS production can indirectly lead to decrease in the levels of autophagy observed under nutrient starvation condition. Thus, contrary to previous reports, NF-κB acts as an autophagy-enhancer under the non-starvation conditions most prevalent in multicellular organisms. Therefore, agents that inhibit the activity of the components of NF-κB (NFKB1 and RELA) act as inhibitors of autophagy and are useful for the treatment of cancer and/or pancreatitis.

### Example 8

### The Function of Reactive Oxygen Species (ROS) in Regulation of Autophagy

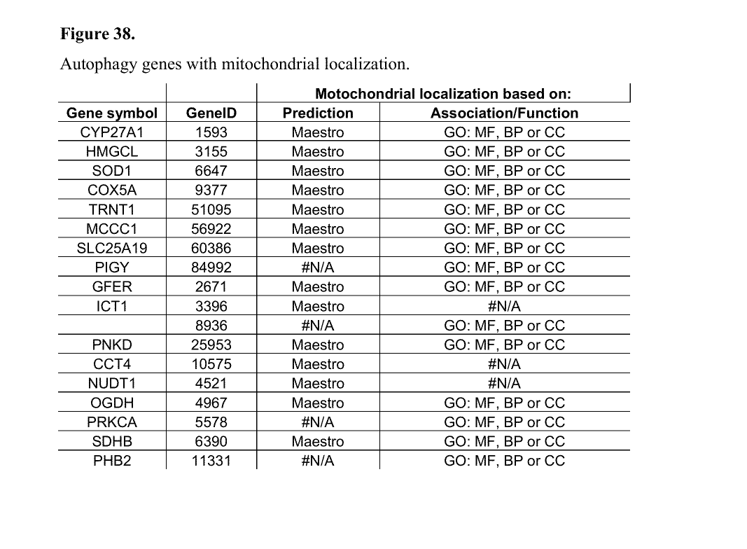

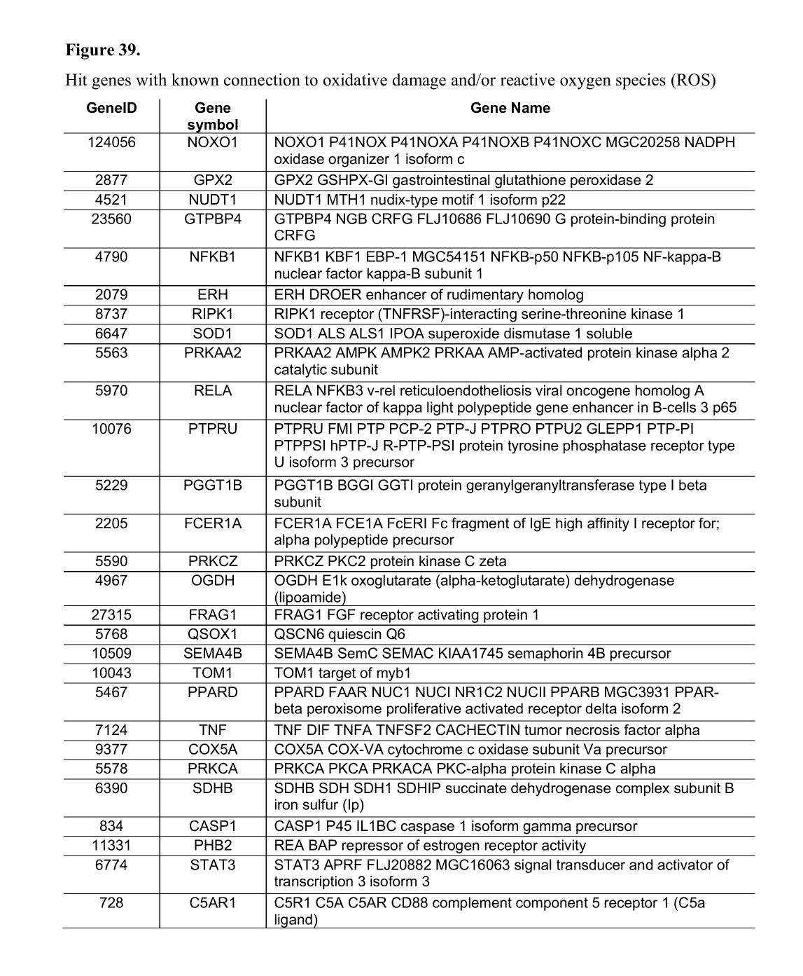

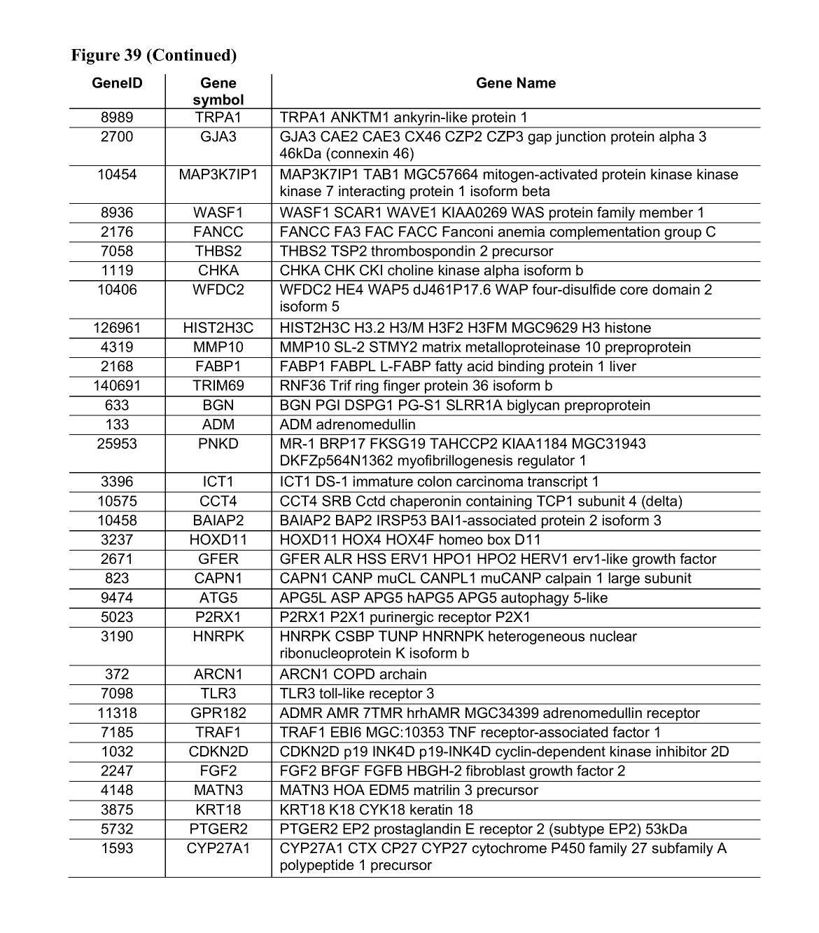

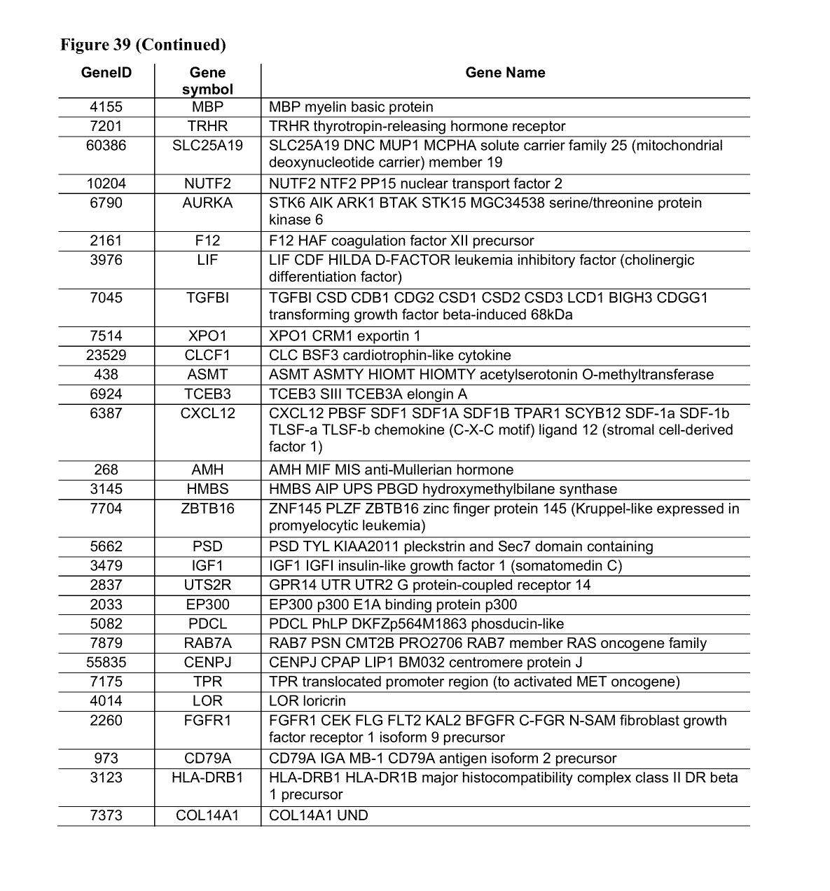

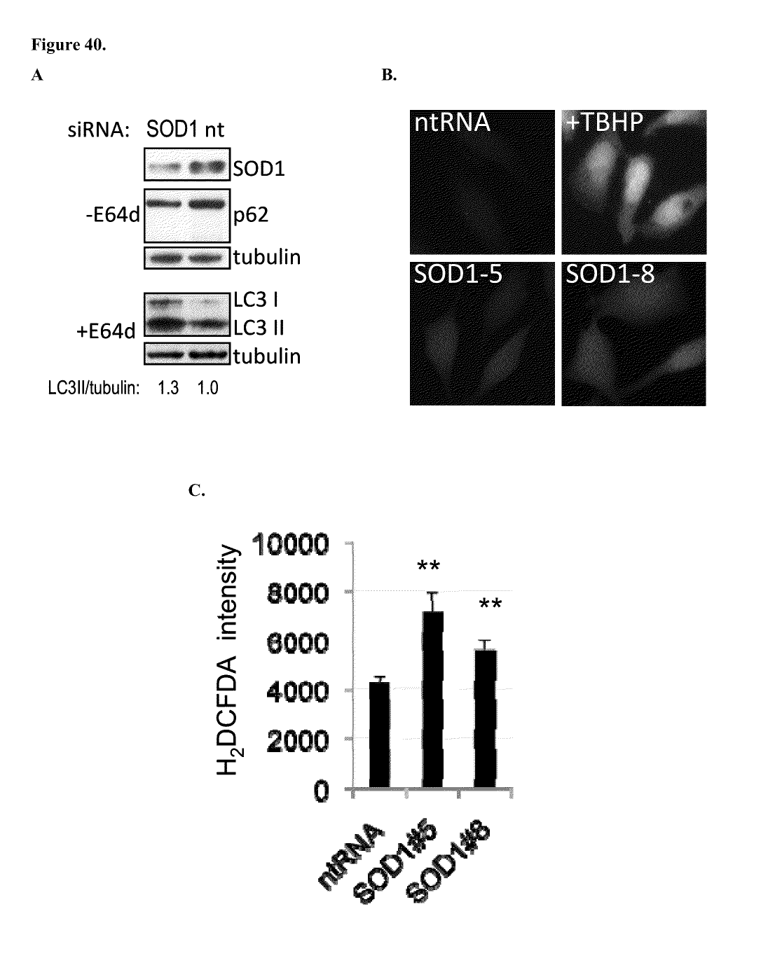

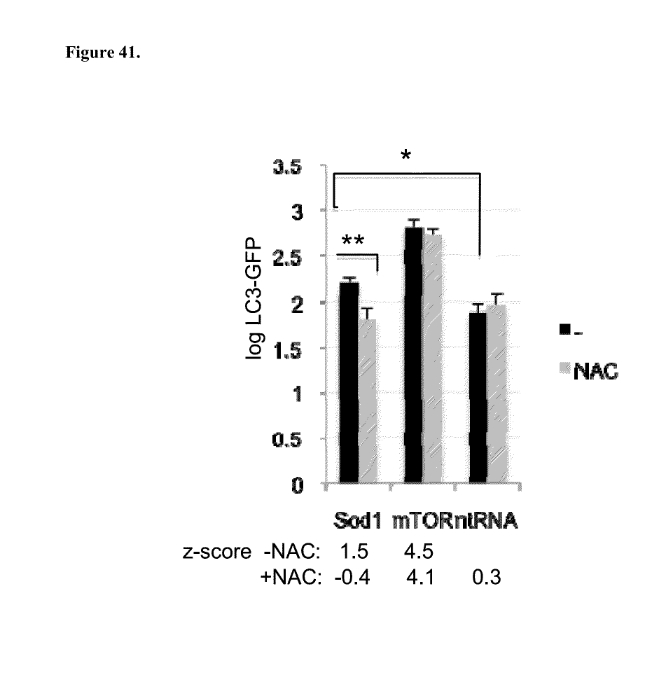

Genes that induce autophagy when knocked-down included SOD1 and GPx2, the major components of the ROS detoxification pathway, as well as several mitochondrial proteins, many of them involved in oxidative respiration and electron transport (FIG. 38). Inhibition of the activity of any of these genes would be expected to lead to the up-regulation of the levels of ROS by either increasing their production or blocking their degradation. Furthermore, many additional screen hits have been reported to be involved in the regulation or to be regulated by ROS (FIG. 39). In order to evaluate a possible role of ROS as a general mediator of autophagy, it was first confirmed that transfection of SOD1 siRNA led to both the induction of autophagy as well as elevated levels of ROS (FIG. 40). Confirming a causal role of ROS, treatment with the antioxidant NAC significantly attenuated induction of autophagy caused by knock-down of Sod1 (FIG. 41). Therefore, interference with normal cellular ROS homeostasis is sufficient for the induction of autophagy.

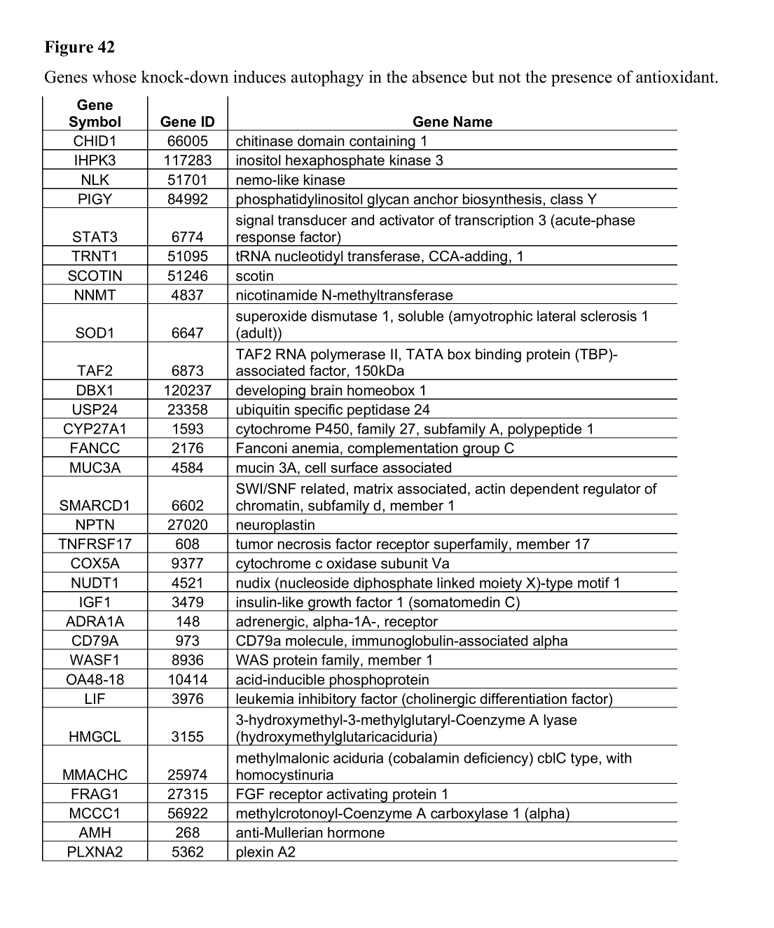

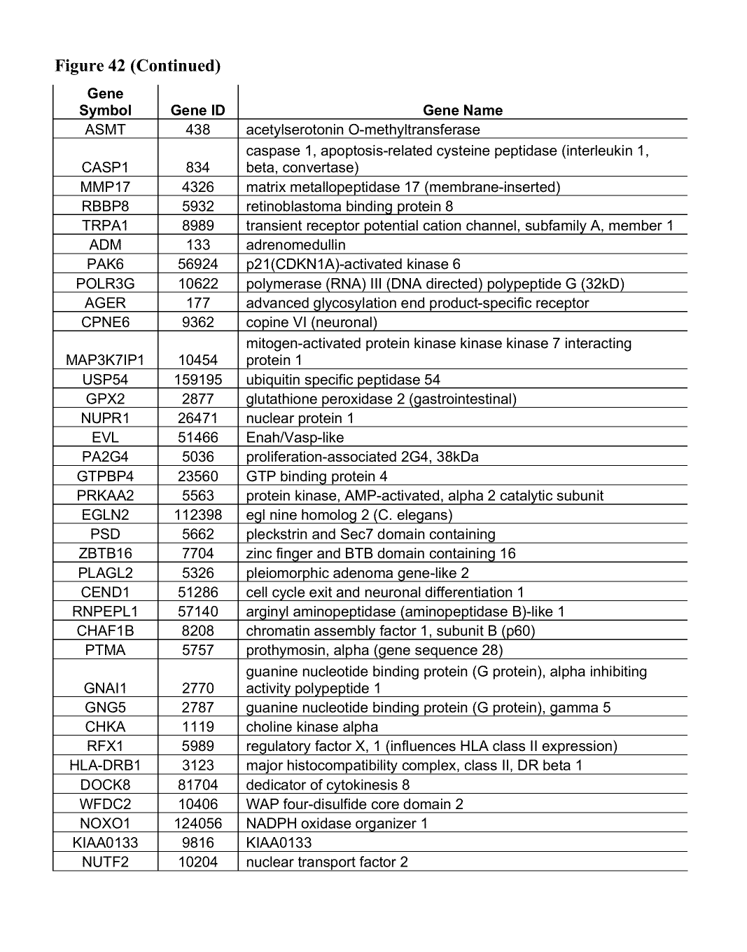

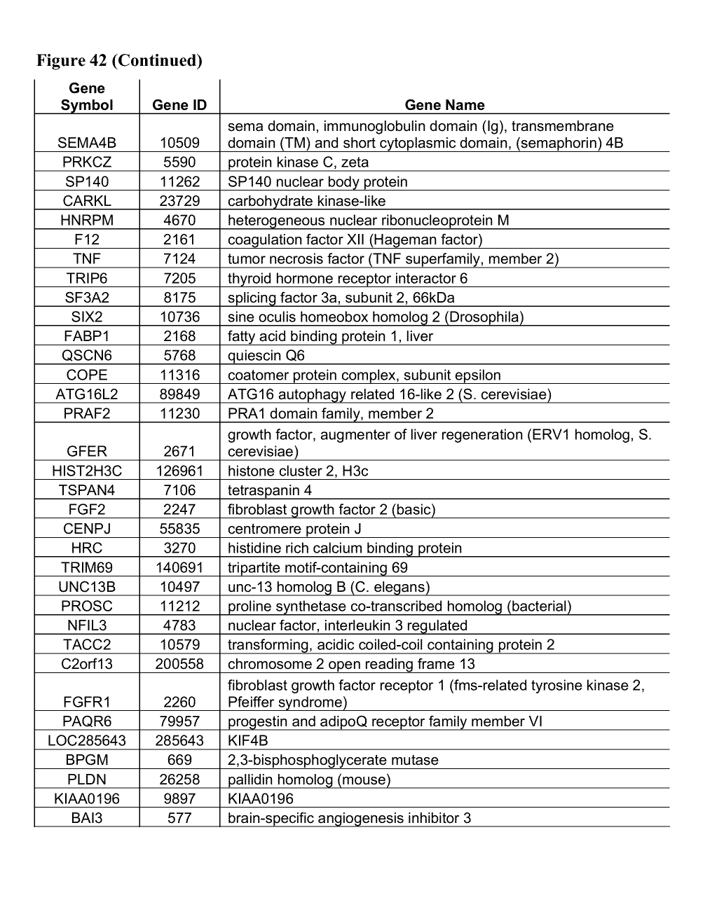

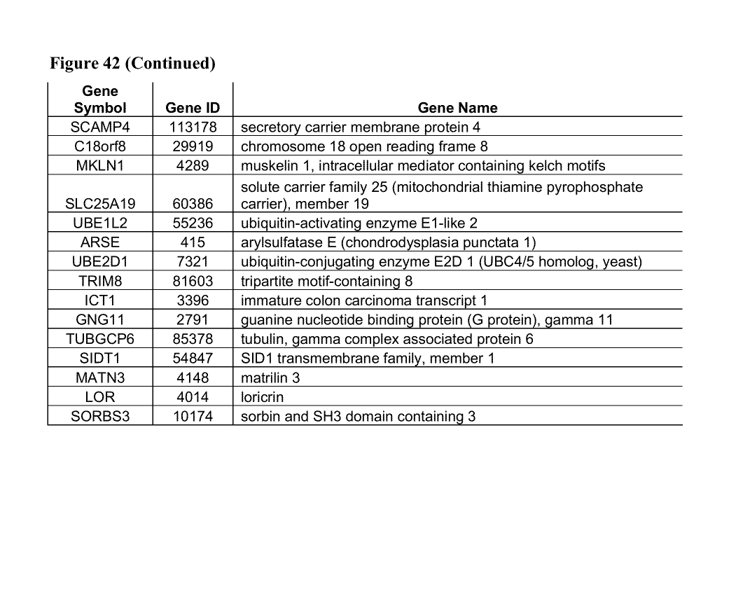

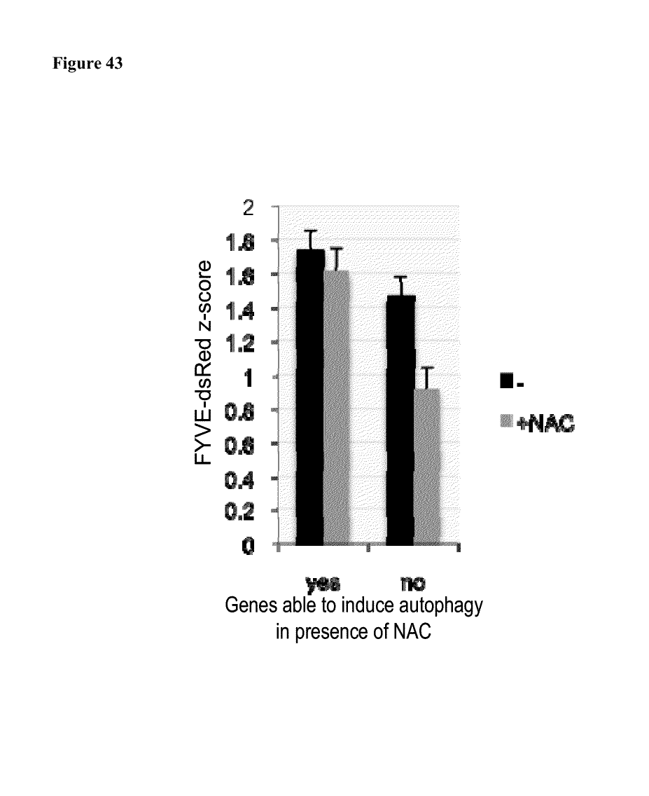

In order to determine if ROS may have a general signaling role during induction of autophagy, a tertiary characterization screen to compare levels of autophagy and type III PI3 kinase activity induced by knock-down of our hit genes in the presence and absence of NAC was performed. Knocking-down a group of the confirmed genes (117, or 54% of all genes tested) led to vesicular LC3-GFP accumulation in the absence but not the presence of the antioxidant, indicating that ROS were required for the induction of autophagy (FIG. 42). Knock-down of these genes also largely failed to increase the accumulation of vesicle-associated FYVE-dsRed in the presence of NAC (FIGS. 42 and 43). This indicates that ROS serve a general function in activation of the type III PI3 kinase, implicating them as important signaling molecules in the early steps of the autophagic pathway.

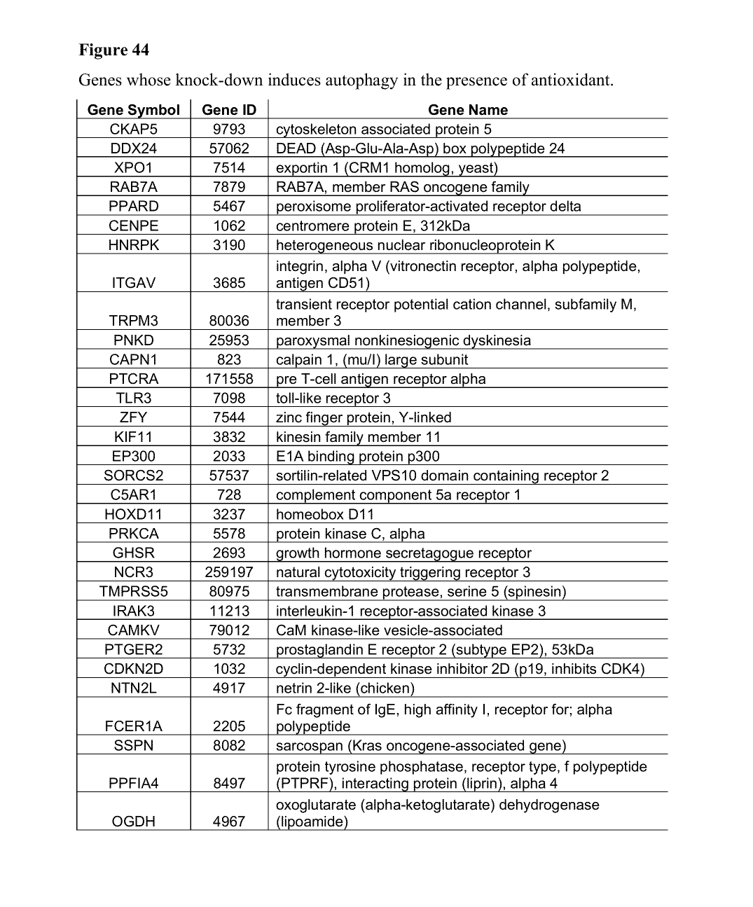

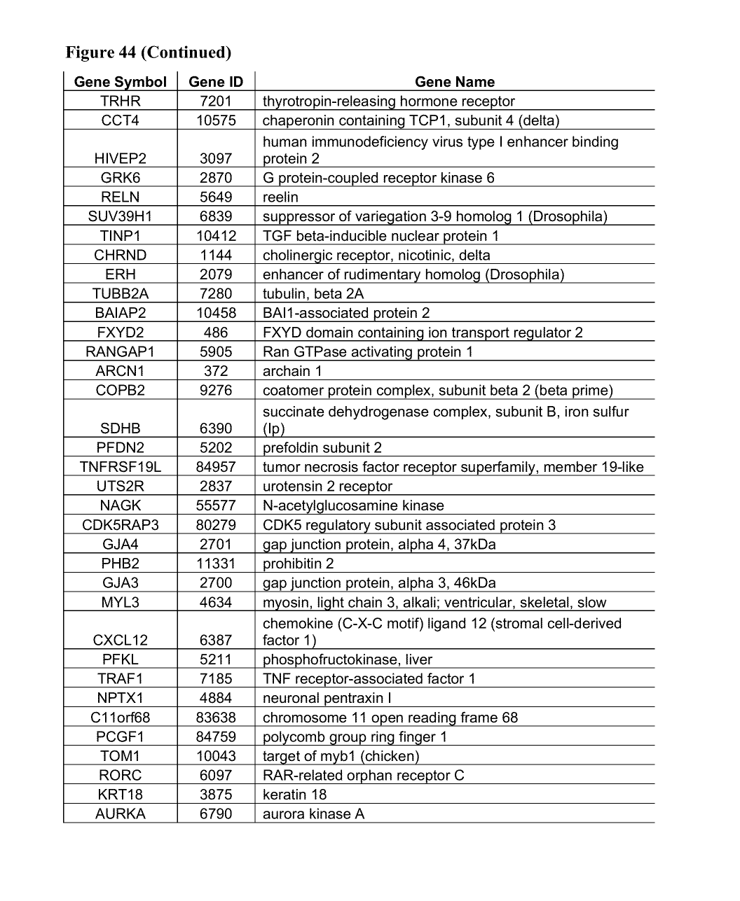

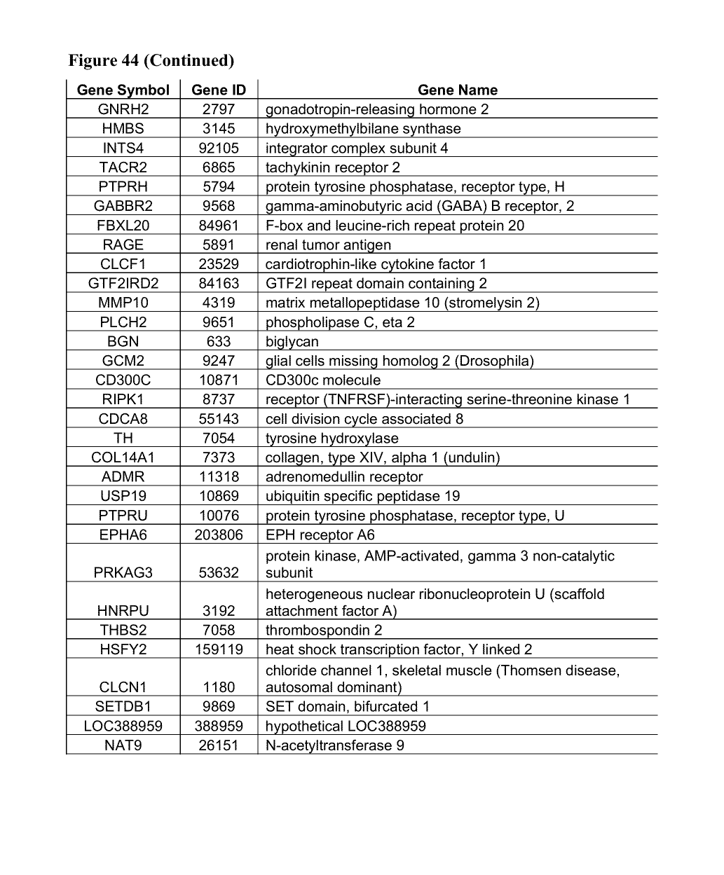

On the other hand, inhibition of the activity of the remaining 98 (46%) genes was able to induce accumulation of LC3-GFP in the presence of NAC, indicating that, in these cases, autophagy can be induced independently of ROS (FIG. 44). Knock-down of these genes was also able to induce comparable average levels of vesicular FYVE-dsRed in the presence and absence of NAC (FIG. 43). Thus, inhibition of the activity of this group of genes led to induction of the type III PI3 kinase through a mechanism independent of ROS.

### Example 9

### Growth Promoting Pathways Negatively Regulate Autophagy

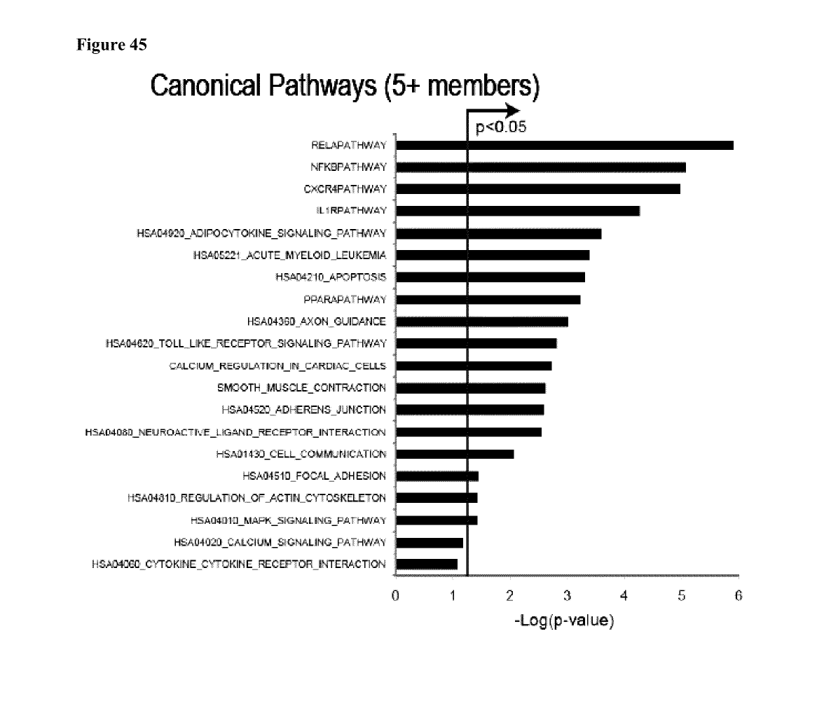

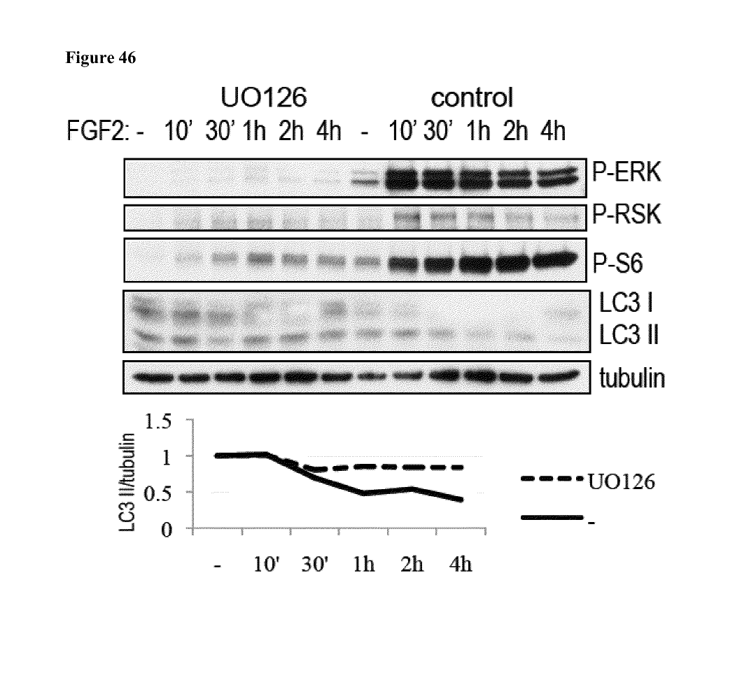

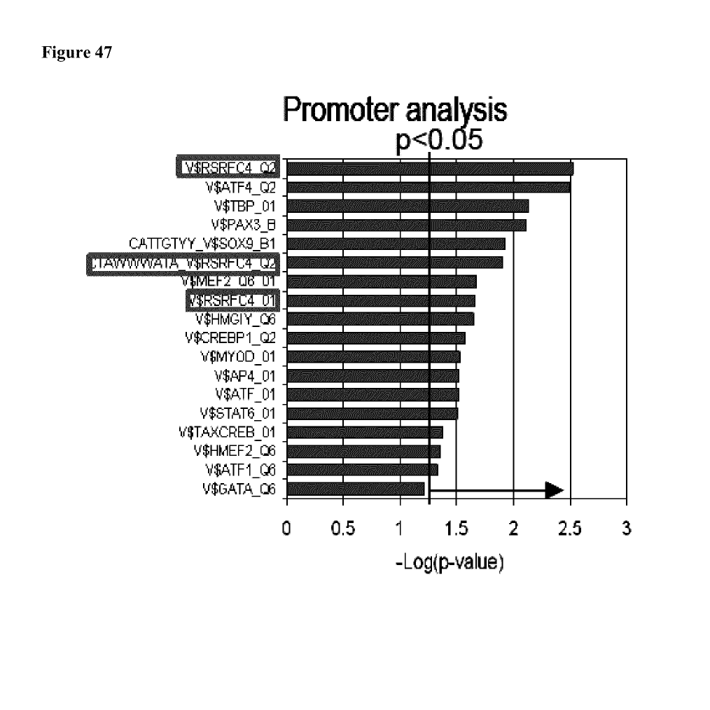

Bioinformatics analysis of the autophagy screen hits indicated significant enrichment for several canonical pathways known to mediate signaling from cell surface receptors (FIG. 45). These pathways included the MAPK (p=0.039), Stat3 (p=0.008) and CXCR4 (p=1.1×10⁻⁵) pathways regulated by the cytokines identified in the screen. FGF2 is known to activate the MAPK pathway and an increased level of phospho-ERK1/2 and phospho-RSK were observed following treatment with FGF2 (FIG. 46). Confirming the essential function of the MAPK pathway, pre-treatment with UO126, an inhibitor of MEK, attenuated inhibition of autophagy following addition of FGF2 (FIG. 46). Additionally, analysis of the promoter regions of all the hit genes revealed significant enrichment in consensus sites for several transcription factors (FIG. 47), including 3 enriched sites for RSRFC4, a member of the serum response factor (SRF) family and a downstream target of MAPK signalling, suggesting additional involvement of transcriptional regulation by the MAPK pathway in control of autophagy under normal growth conditions.

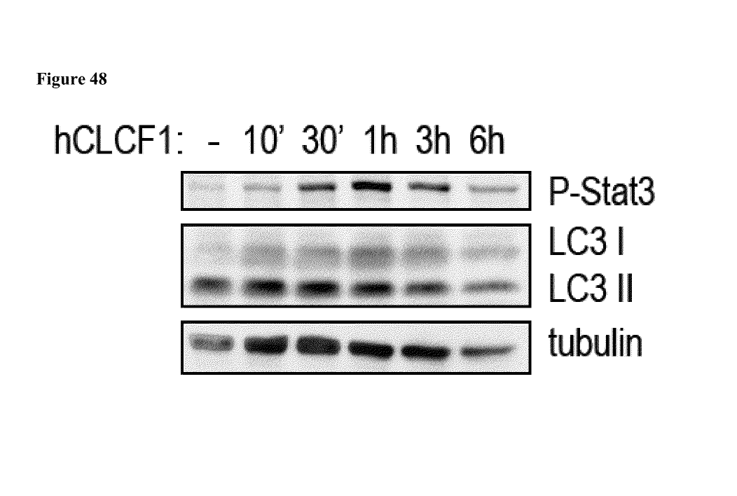

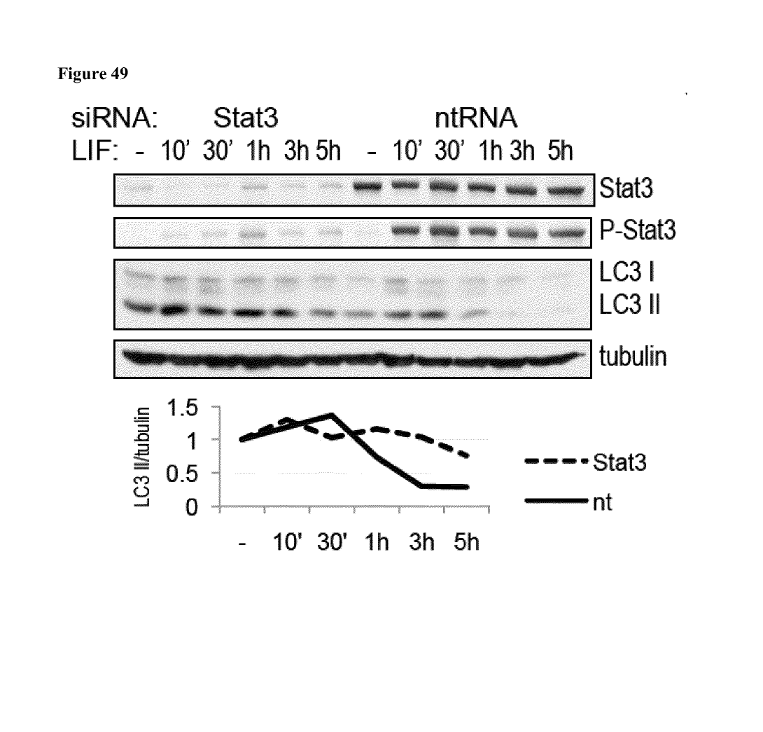

Another hit gene pulled out of the screen as a negative regulator of autophagy was the transcription factor Stat3, a mediator of LIF and CLCF1 signaling. Indeed, treatment with either LIF or CLCF1 increased activating phosphorylation of Stat3 (FIGS. 48 and 49). Consistent with the essential function of Stat3, its siRNA mediated knock-down attenuated down-regulation of autophagy in response to LIF (FIG. 49). Therefore, LIF and CLCF1 regulate autophagy through the Stat3 pathway.

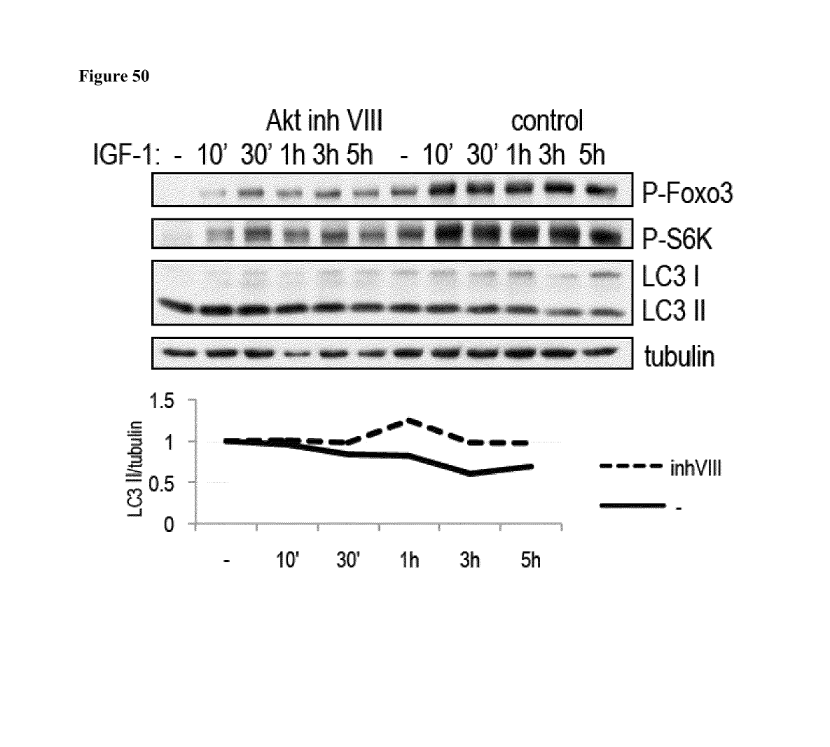

In addition to activating mTORC1, Akt directly phosphorylates and inhibits Foxo3a, a transcription factor that positively regulates autophagy during muscle degeneration. Indeed, phosphorylation of both Akt and Foxo3a was increased following IGF-1 treatment in both the absence and presence of rapamycin (FIG. 50). Inhibition of Akt by treatment with Akt inhibitor VIII attenuated phosphorylation of both Foxo3a and the mTORC1 target S6 kinase, as well as prevented inhibition of autophagy by IGF1 (FIG. 50). Therefore, under normal nutrient conditions IGF-1 regulates autophagy in a type I PI3 kinase/Akt dependent manner, likely through both the mTORC1 and Foxo3a pathways.

### Example 10

### The Down Regulation of Autophagy During Human Aging

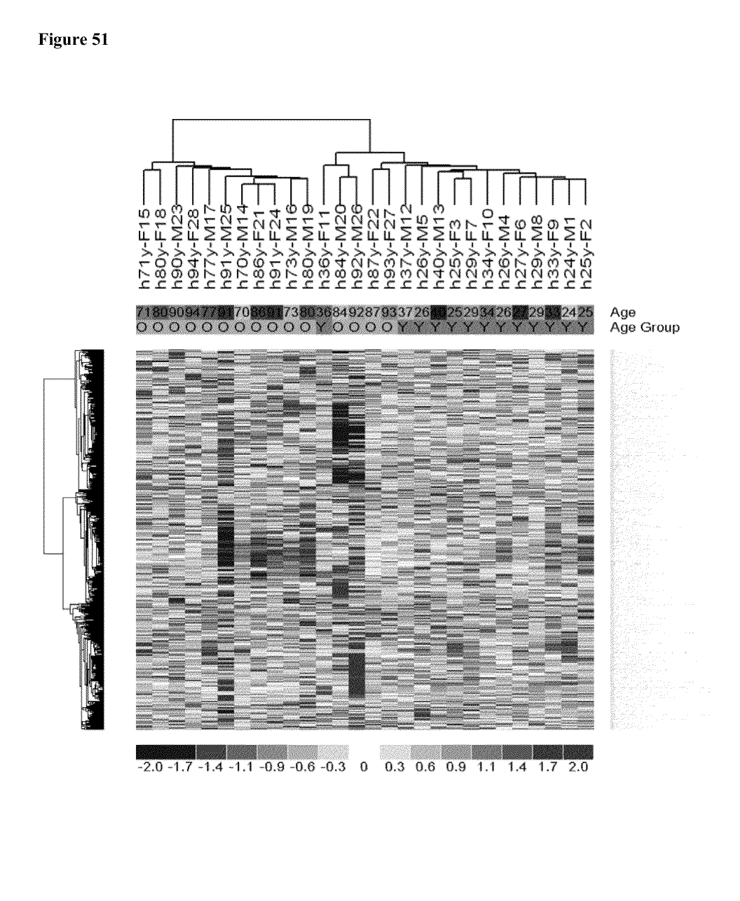

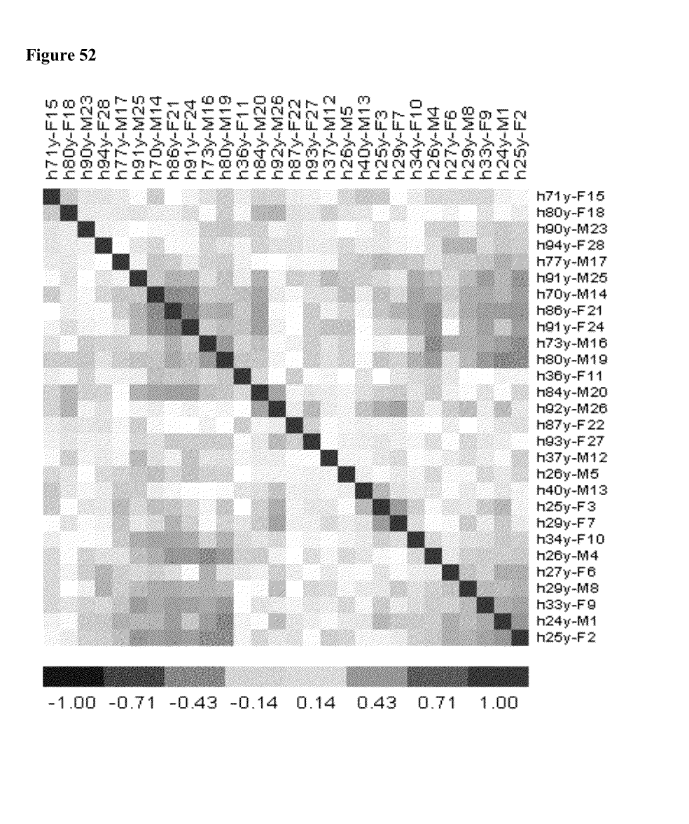

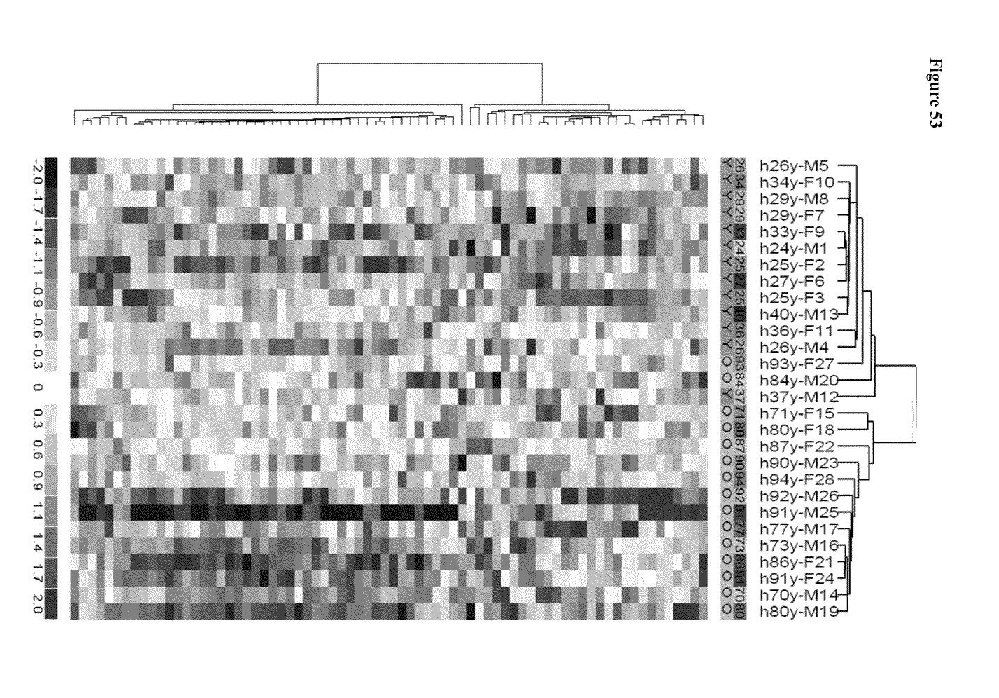

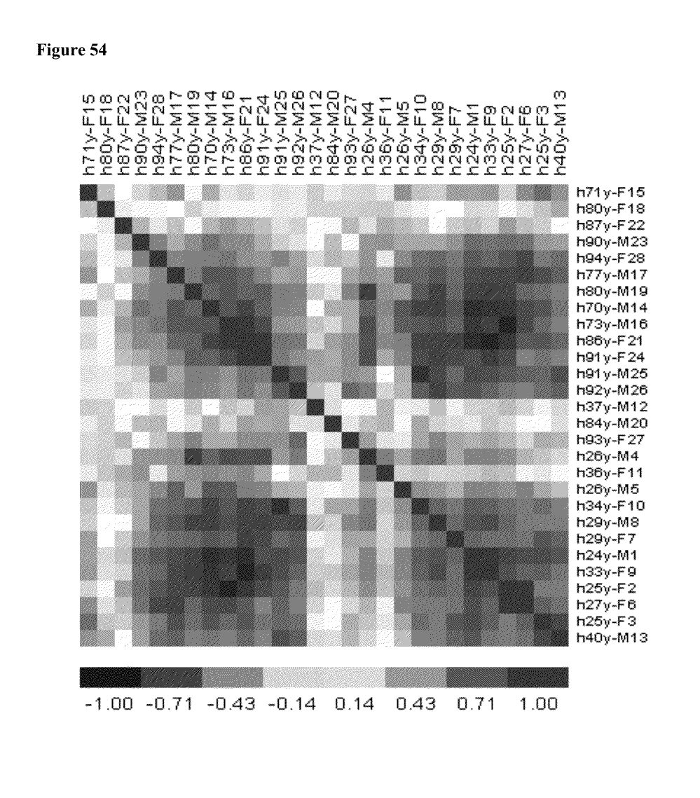

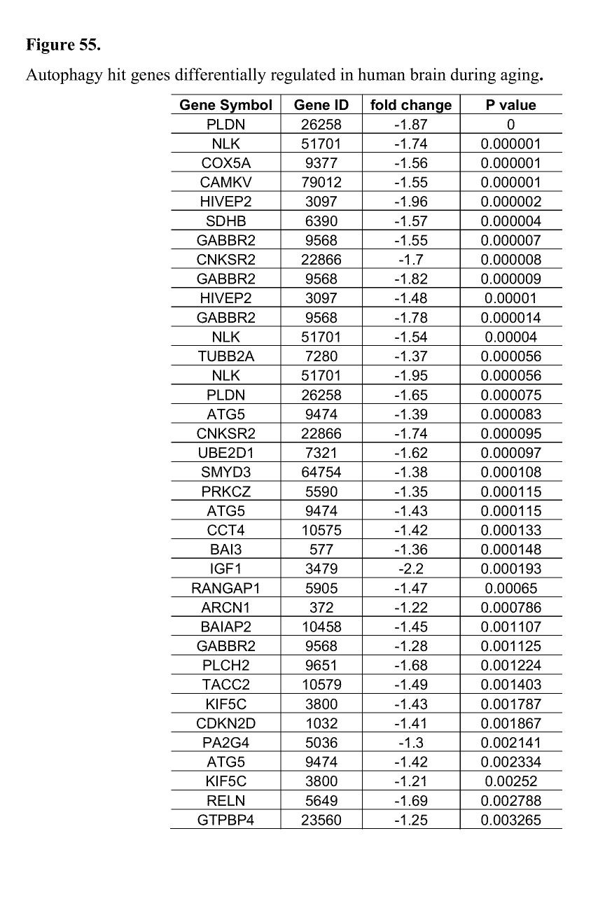

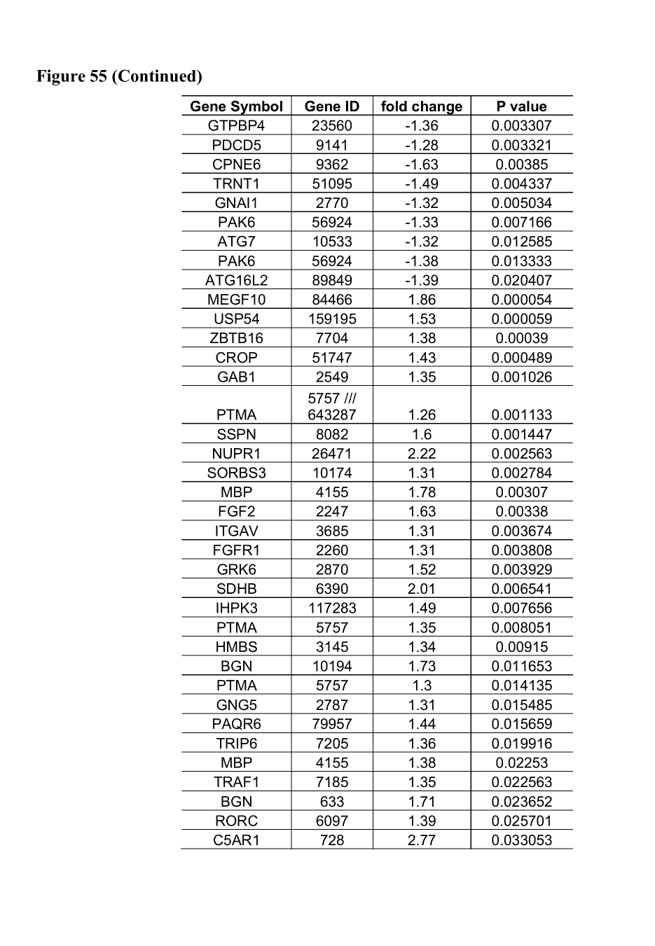

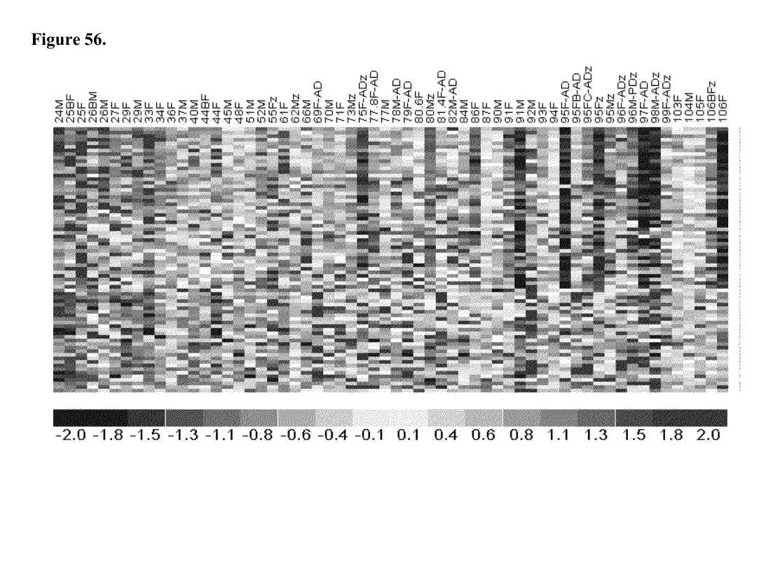

In order to specifically address the potential function of the autophagy-related genes in neurodegeneration associated with aging, the mRNA expression of the autophagy hit genes were analyzed in a set of young versus old human brain samples. Differential expression of a large subset of genes (FIGS. 51 and 52) was observed, including a groups of 32 genes significantly (p<0.05) up-regulated and 46 genes significantly down-regulated with age (FIG. 53-55). Interestingly, gene ontology (GO) biological process analysis revealed that the age up regulated group was highly enriched in genes involved in mediation and regulation of the MAPK pathway (p=1.6×10⁻⁴), the increased activity of which is predicted by our analysis to lead to the suppression of autophagy. Conversely, expression of the key autophagy genes, Atg5 and Atg7, was down regulated during aging (FIG. 55). These data suggest that differential gene expression leads to the down regulation of autophagy in the brain during aging, which would contribute to development of chronic neurodegenerative diseases. Consistent with this hypothesis, further analysis in a more extensive set of samples, including those from middle-aged individuals, revealed that Atg5 and Atg7 were among a group of genes necessary for the mediation of autophagy in mammalian cells whose expression was gradually down-regulated in an age-dependent manner starting in the early sixties (FIG. 56), which is often the earliest age of onset for the sporadic neurodegenerative diseases such as Alzheimer's Disease (AD). Therefore, age-dependent regulation of genes identified in our screen likely contributes to down-regulation of autophagy during normal human aging, and thus useful as therapeutic targets to prevent and treat age-related neurodegenerative diseases.

### Example 11

### Differential Expression of Autophagy Regulators in Alzheimer's Disease Brain samples

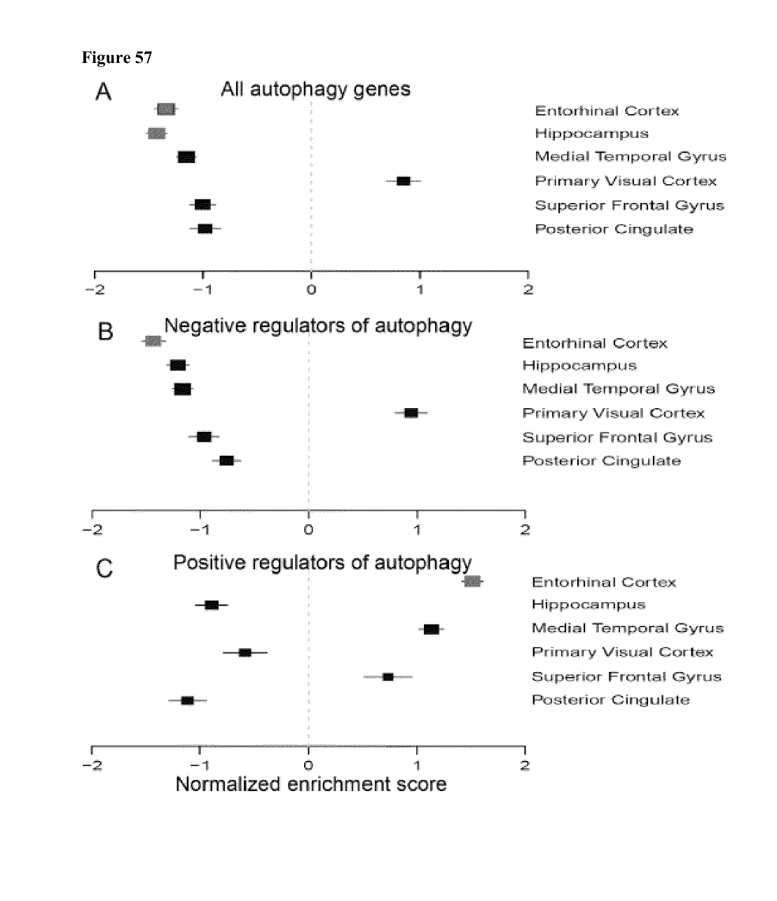

Accumulation of both ROS and autophagic vesicles (AV) are early features in AD. To determine if we can detect changes in the expression of genes involved in regulation of autophagy in this disease, the expression of the autophagy screen hit genes from six brain regions of 34 cases with AD and 14 age-matched normal controls were analyzed. An overall significant under-expression of the hit genes in AD patient samples compared to controls specifically in the hippocampus and entorhinal cortex, the brain regions most affected by the disease, were observed (FIG. 57A). Consistent trends were observed in other brain regions affected by AD (superior frontal gyrus, posterior cingulate, and medial temporal gyms). Notably, in the visual cortex, a brain region relatively resistant to AD pathology, these changes were absent. Further sub-division of the hit genes revealed that in the entorhinal cortex negative regulators of autophagy flux were specifically negatively enriched (FIG. 57B). A similar trend was also observed in other brain areas affected by AD. Conversely, positive regulators of autophagy were positively enriched in the entorhinal cortex (FIG. 57C). Such differential expression patterns of autophagy regulators suggest up-regulation of autophagy in AD brains.

### Example 12

### ROS Mediate Autophagy in Response to Amyloid β

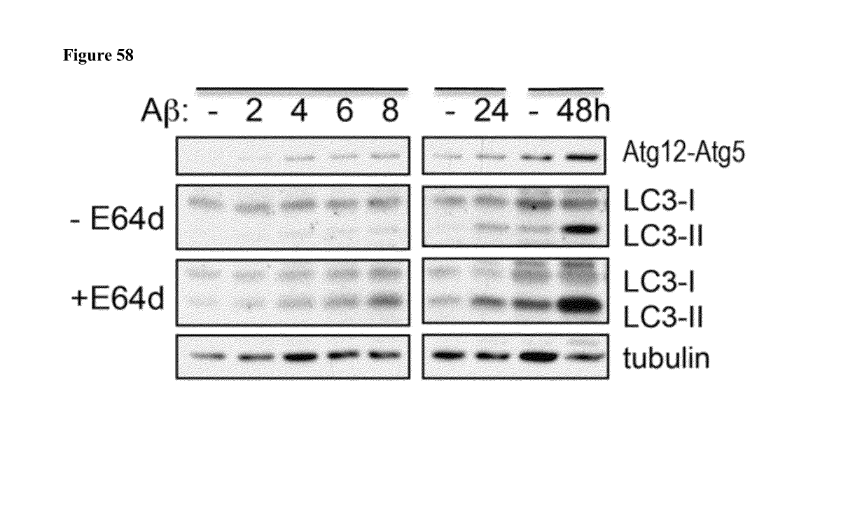

Amyloid β (Aβ) is the main pathogenic factor in AD. Whether induction of autophagy by Aβ was be mediated by ROS was examined. Following treatment of H4 cells with Aβ, increased levels of autophagy were observed (FIG. 58). In order to determine if this was due to an increase in the initiation of autophagy or to a block in lysosomal degradation, the accumulation of LC3-II following Aβ treatment in the absence and presence of lysosomal protease inhibitor E64d was observed (FIG. 58). Up to 8 hours after treatment, the accumulation of LC3-II could be observed only in the presence of E64d. At 48 hrs after the addition of Aβ, the increased levels of LC3-II were observed even without E64d, but were further increased in the presence of E64d, Additionally, increased conjugation of Atg12-Atg5 starting 4 hours after Aβ treatment was observed. Together these data indicate increased initiation of autophagy in response to Aβ.

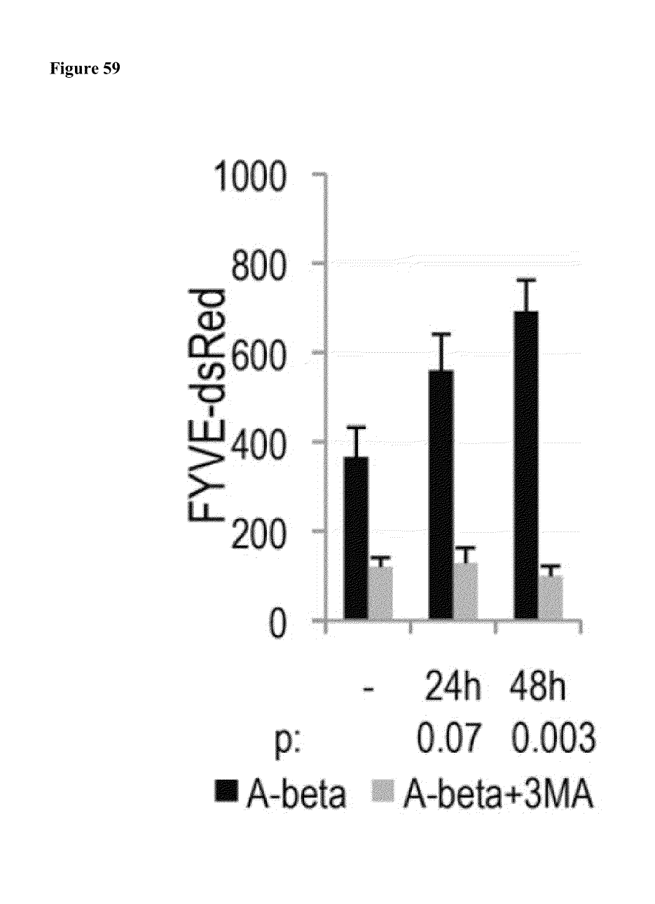

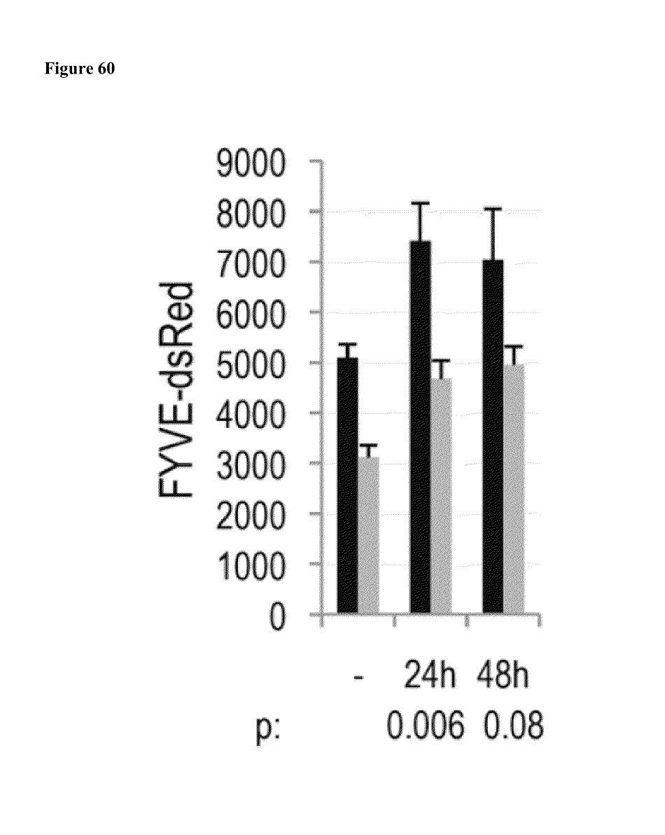

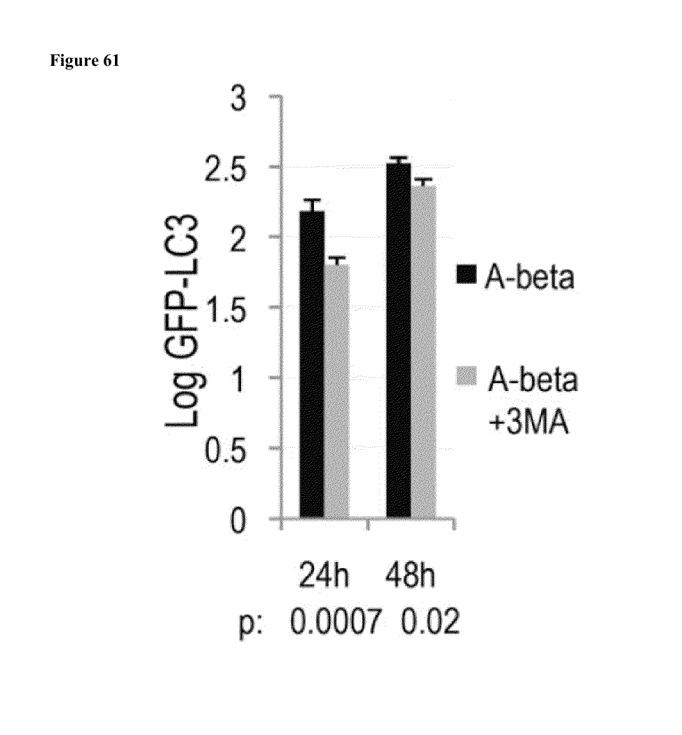

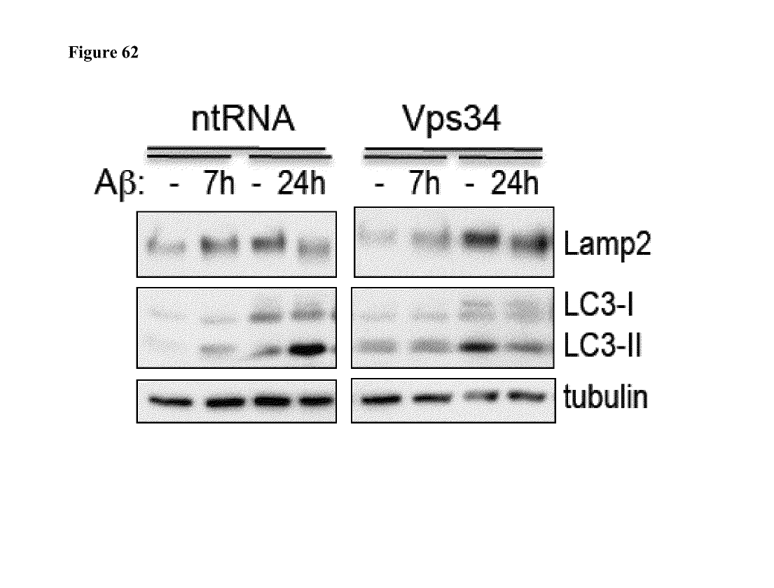

The involvement of type III PI3 kinase in the induction of autophagy by Aβ was investigated. Accumulation of PtdIns3P was observed, which was suppressed in the presence of 3MA (FIG. 59), confirming the involvement of the type III PI3 kinase. In agreement with a causal role of ROS, accumulation of PtdIns3P was suppressed in the presence of NAC (FIG. 60). Finally, treatment with 3MA (FIG. 61) or knock down of Vps34 (FIG. 62) was able to attenuate induction of autophagy in response to Aβ.

### EQUIVALENTS

The present invention provides, methods for the modulation of autophagy and the treatment of autophagy related diseases. While specific embodiments of the subject invention have been discussed, the above specification is illustrative and not restrictive. Many variations of the invention will become apparent to those skilled in the art upon review of this specification. The appended claims are not intended to claim all such embodiments and variations, and the full scope of the invention should be determined by reference to the claims, along with their full scope of equivalents, and the specification, along with such variations.

All publications and patents mentioned herein are hereby incorporated by reference in their entirety as if each individual publication or patent was specifically and individually indicated to be incorporated by reference. In case of conflict, the present application, including any definitions herein, will control.

What is claimed is:

1. A method of inhibiting autophagy in a cell comprising contacting said cell with an agent that inhibits the activity of a product of a gene selected from the group consisting of the genes listed in Table 2.

2. The method of claim 1, wherein the gene is selected from the group consisting of the genes listed in Table 4.

3. The method of claim 1, wherein said gene is selected from the group consisting of the genes listed in Table 6.

4. The method of claim 1, wherein said agent is a siRNA, shRNA or antisense RNA molecule.

5. The method of claim 3, wherein said gene is TPR or GPR18.

6. The method of claim 5, wherein said agent is an antibody specific for the product of said gene.

7. The method of claim 1, wherein said gene is RelA or NFκB.

8. The method of claim 7, wherein said gene is RelA.

9. A method of inducing autophagy in a cell comprising contacting said cell with an agent that enhances the activity of a product of a gene selected from the group consisting of the genes listed in Table 2 (hits that decrease autophagy).

10. The method of claim 9, wherein said gene is selected from the group consisting of the genes listed in Table 4.

11. The method of claim 9, wherein said gene is selected from the group consisting of the genes listed in Table 6.

12. The method of claim 11, wherein said gene is TPR or GPR18.

13. The method of claim 12, wherein said agent is an antibody specific for the product of said gene.

14. The method of claim 9, wherein said gene is RelA or NFκB.

15. The method of claim 14, wherein said gene is RelA.

16. A method of treating a neurodegenerative disease in a subject comprising administering to said subject an agent that enhances the activity of a product of a gene selected from the group consisting of the genes listed in Table 2.

17-50. (canceled)
